(19) United States
(12) Patent Application Publication
(10) Pub. No.: US 2025/0171516 A1
(43) Pub. Date: May 29, 2025

(54) PEPTIDE ACIDS AS A GLP1R AGONIST AND ANTIBODY-DRUG CONJUGATES THEREOF

(71) Applicant: Regeneron Pharmaceuticals, Inc., Tarrytown, NY (US)

(72) Inventors: Amy HAN, Hockessin (DE); Xiang Zheng, Allendale, NJ (US); William Olson, Yorktown Heights, NY (US); Andrew J. Murphy, Croton-on-Hudson, NY (US); Frank Delfino, Poughquag, NY (US); Haruka Okamoto, Ardsley, NY (US); Jean Yanolatos, Bronx, NY (US); Huachuan Cao, Briarcliff Manor, NY (US); Mark W. Sleeman, Carmel, NY (US)

(73) Assignee: Regeneron Pharmaceuticals, Inc., Tarrytown, NY (US)

(21) Appl. No.: 18/934,716

(22) Filed: Nov. 1, 2024

Related U.S. Application Data
(60) Provisional application No. 63/596,063, filed on Nov. 3, 2023.
(60) Provisional application No. 63/556,504, filed on Feb. 22, 2024.
(60) Provisional application No. 63/694,339, filed on Sep. 13, 2024.

(51) Int. Cl. C07K 14/605; A61K 47/54; A61K 47/60; A61K 47/68; A61P 3/10

(52) U.S. Cl. CPC C07K 14/605; A61K 47/545; A61K 47/60; A61K 47/6811; A61K 47/6849; A61K 47/6889; A61P 3/10

(57) ABSTRACT

Described herein are protein-drug conjugates and compositions thereof that are useful, for example, for targeting glucagon-like peptide 1 receptor (GLP1R). In certain embodiments, provided are peptidomimetic payloads and linker-payloads and methods of making same. More specifically, GLP1 peptidomimetics, antibody-drug conjugates, and compositions which comprise anti-GLP1R antibodies and GLP1 peptidomimetic payloads and methods of treating GLP1R-associated conditions are provided.

### CROSS-REFERENCE TO RELATED APPLICATIONS

This application claims the benefit of U.S. Provisional Application Nos. 63/694,339, filed Sep. 13, 2024, 63/556,504, filed Feb. 22, 2024, and 63/596,063, filed Nov. 3, 2023, the disclosure of each of which is incorporated by reference herein in its entirety.

### SEQUENCE LISTING

The instant application contains a Sequence Listing which has been submitted electronically in XML file format and is hereby incorporated by reference in its entirety. Said XML copy, created on Oct. 18, 2024, is named 250298_000729_SL.xml and is 764,371 bytes in size.

### FIELD OF THE DISCLOSURE

The present disclosure relates to protein-drug conjugates (e.g., antibody-drug conjugates), pharmaceutical compositions, and methods of treating disease therewith. Also provided are peptidomimetic payloads and linker-payloads and methods of making same. More specifically, the present disclosure relates to protein-drug conjugates (e.g., antibody-drug conjugates) comprising GLP1 peptidomimetics and methods of treating GLP1R-associated conditions therewith.

### BACKGROUND OF THE DISCLOSURE

Diabetes is a chronic disease of abnormal glucose metabolism. 425 million people are estimated to be living with diabetes worldwide. Global diabetes drugs include insulin, DPP-4 inhibitors, glucagon-like peptide 1 receptor (GLP1R) agonists, but most patients do not achieve combined treatment goal to manage hyperglycaemia and cardiovascular risk factors.

Glucagon-Like Peptide 1 Receptor (GLP1R) is the receptor for glucagon-like peptide 1 (GLP1) and is expressed in the pancreatic beta cells. GLP1R is also expressed in the brain where it functions in the control of appetite, memory, and learning. GLP1R is a member of the secretin family (Class B) of G protein-coupled receptors (GPCRs). Upon binding of its ligand, GLP1, GLP1R initiates a downstream signaling cascade through Gαs G-proteins that raises intracellular cyclic AMP (cAMP) levels, which leads to the transcriptional regulation of genes (Donnelly 2011). Activation of GLP1R results in increased insulin synthesis and release of insulin.

GLP1R and GLP1 are highly validated targets for obesity and type 2 diabetes. Marketed GLP1R agonists increase insulin secretion, thereby lowering blood glucose levels, but they require weekly or more frequent administration.

Accordingly, there is a need in the art for GLP1R agonists with longer duration and better safety. In certain embodiments, the present disclosure meets the needs and provides other advantages.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE DISCLOSURE

Various non-limiting aspects and embodiments of the disclosure are described below.

In one aspect, compound having a structure of a structure of Formula (P-I), Formula (P-II), Formula (P-III), or Formula (P-IV):

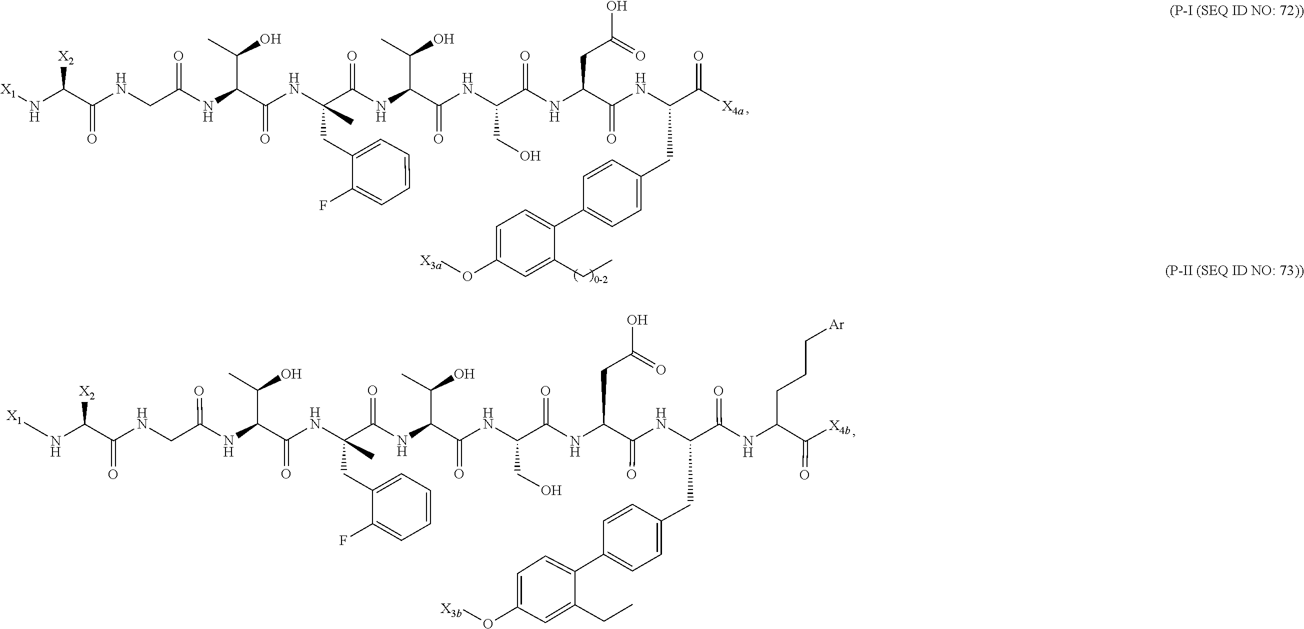

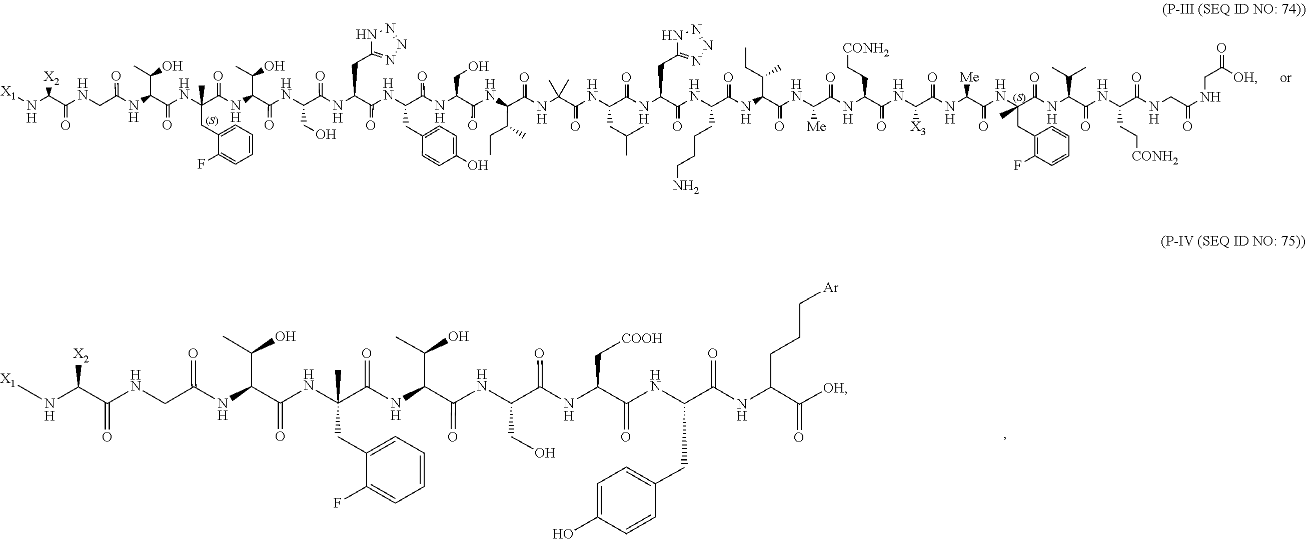

wherein:
X₁ is selected from H;

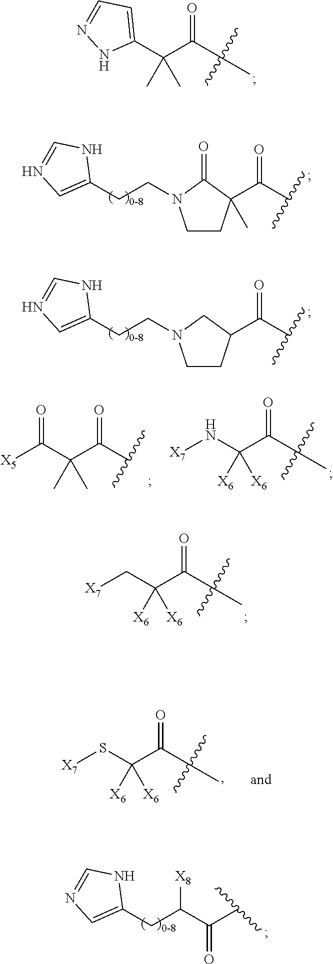

X₂ is selected from

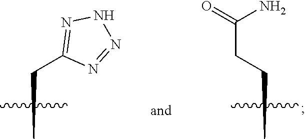

X₃ₐ is selected from —CH₃, —(CH₂)₁₋₄(OCH₂CH₂)₂₋₁₅—NH₂, —(CH₂)₂₋₆—NH₂, —(CH₂)₂₋₆—N₃, and —(CH₂)₂₋₆-Tr-(CH₂)₁₋₆—NH₂;
X_{3b} is selected from —CH₃, —(CH₂)₂₋₆—NH₂, —(CH₂)₂₋₆—N₃, and —(CH₂)₂₋₆-Tr-(CH₂)₁₋₆—NH₂;
Tr is a triazole moiety;
X₄ₐ is —NH₂ or —OH;
X_{4b} is —OH;
X₅ is selected from —OH, —NH₂, —NH—OH, and

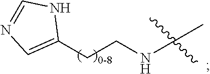

X₆ is independently at each occurrence selected from H, —OH, —CH₃, and —CH₂OH;
X₇ is selected from H,

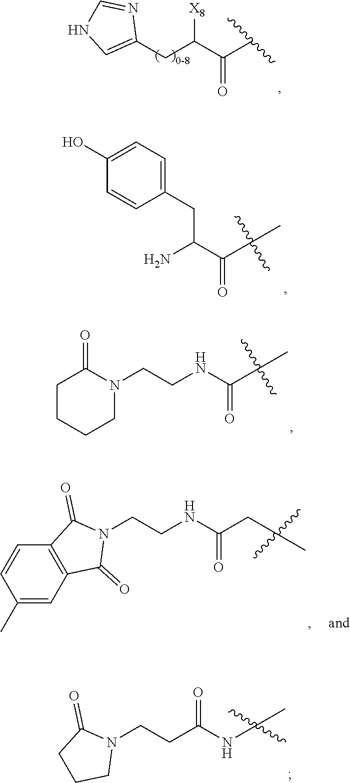

X₈ is selected from H, —OH, —NH₂, and

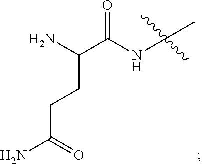 [SMILES: *NC(=O)C(N)CCC(N)=O]

Ar is selected from

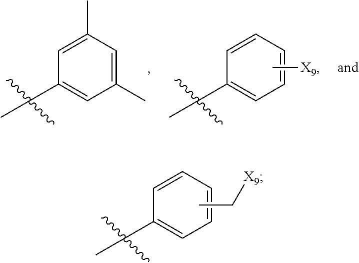

and
X₉ is selected from —NH₂,

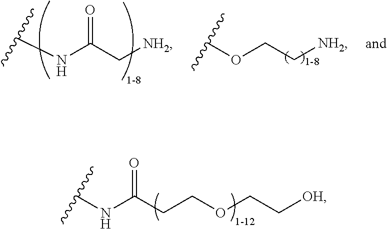

or a pharmaceutically acceptable salt thereof,
with the proviso that when X_{4b} is —OH, and Ar is

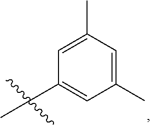 [SMILES: *c1cc(C)cc(C)c1]

### X₅ is not

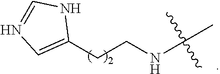

In some embodiments, the compound of Formula (P-I) has a structure of Formula (P-Ia):

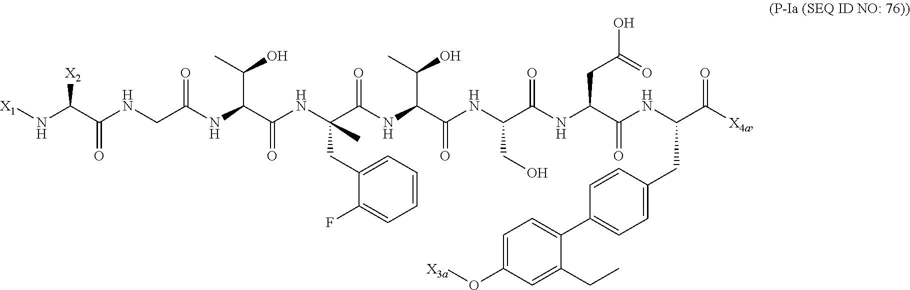

wherein X₃ₐ is selected from —CH₃, —(CH₂)₂₋₆—NH₂, —(CH₂)₂₋₆—N₃, and —(CH₂)₂₋₆-Tr-(CH₂)₁₋₆—NH₂.

In some embodiments, the compound of Formula (P-I) has a structure of Formula (P-Ib):

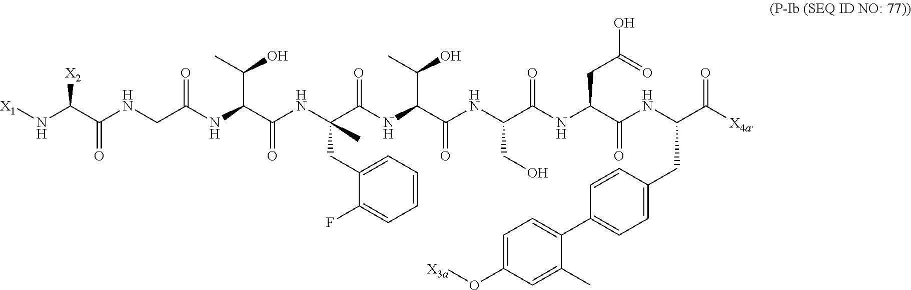

In some embodiments, X₁ is

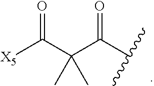

In some embodiments, X₁ is

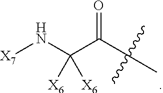

In some embodiments, X₂ is

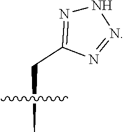

In some embodiments, X₃ₐ is —(CH₂)₂₋₆—N₃.

In some embodiments, X₃ₐ is —(CH₂)₁₋₄(OCH₂CH₂)₂₋₁₅—NH₂.

In some embodiments, X_{3b} is —(CH₂)₂₋₆—N₃.

In some embodiments, X₅ is

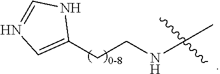

In some embodiments, X₇ is

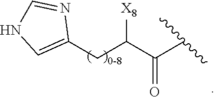

In some embodiments, X₄ₐ is —NH₂.

In some embodiments, X₄ₐ is —OH.

In another aspect, provided herein is a compound having a structure of Formula (LP-I), Formula (LP-II), Formula (LP-II′), or Formula (LP-III):

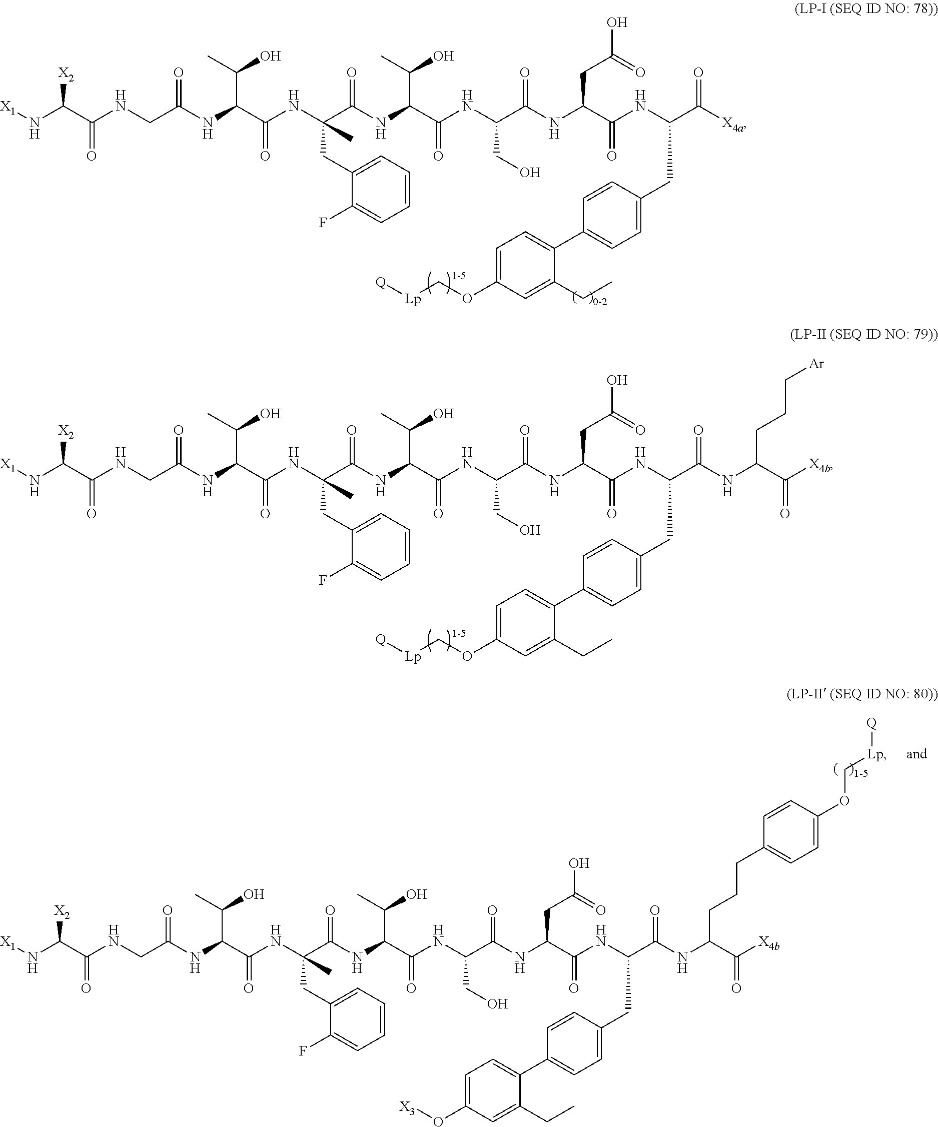

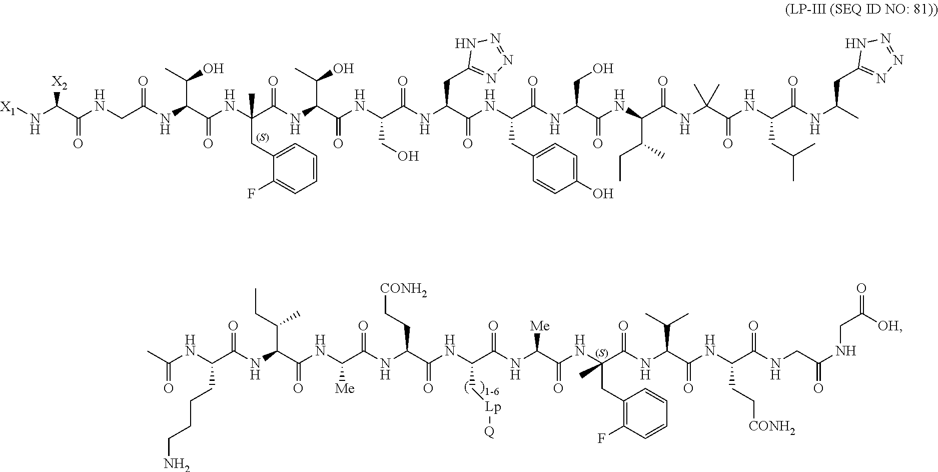

wherein:
X₁ is selected from H;

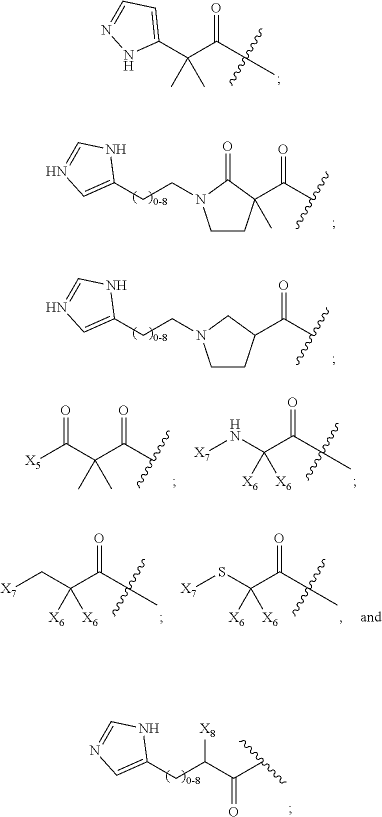

X₂ is selected from

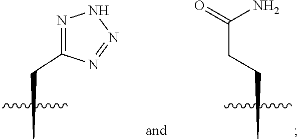

X₃ is selected from —CH₃, —(CH₂)₂₋₆—NH₂, —(CH₂)₂₋₆—N₃, and —(CH₂)₂₋₆-Tr-(CH₂)₁₋₆—NH₂, where
Tr is a triazole moiety;
Lₚ is absent or a linker comprising one or more of

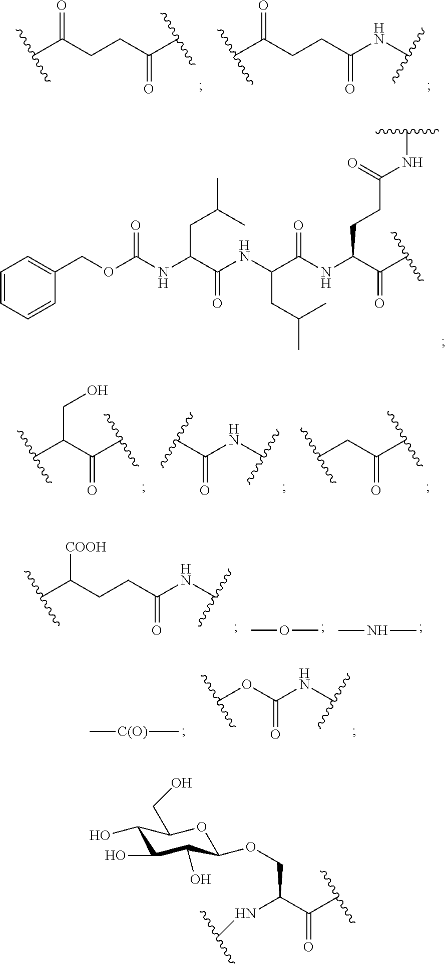

a cyclodextrin; —CH₂—O—; a polyethylene glycol (PEG) segment having 1 to 36-CH₂CH₂O-(EG) units; —(CH₂)₁₋₂₄—; a triazole; one or more amino acids selected from glycine, serine, glutamic acid, alanine, valine, threonine, leucine, and proline, and combinations thereof;
Q is a moiety selected from

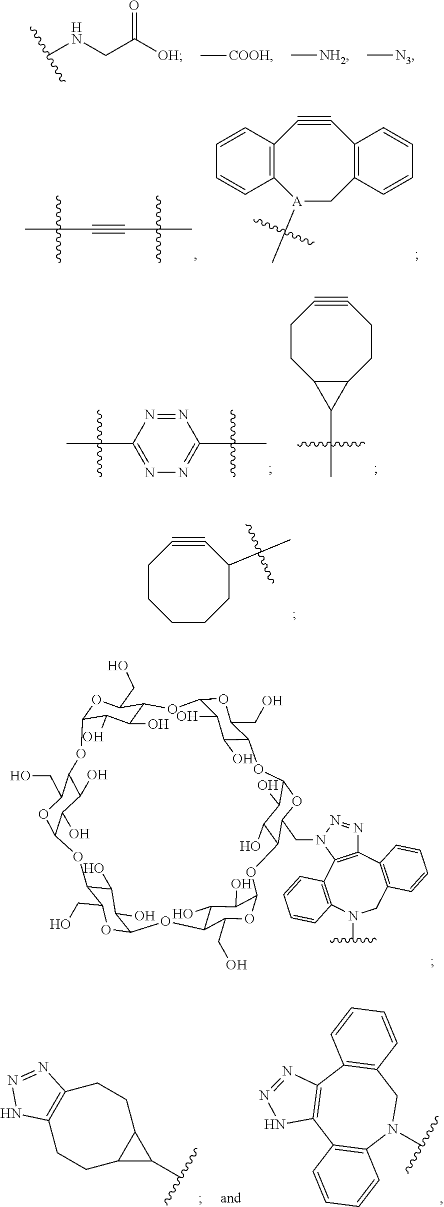

where A is C or N;
X₄ₐ is —NH₂ or —OH;
X_{4b} is —OH;
X₅ is selected from —OH, —NH₂, —NH—OH, and

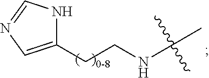

X₆ is independently at each occurrence selected from H, —OH, —CH₃, and —CH₂OH;
X₇ is selected from H,

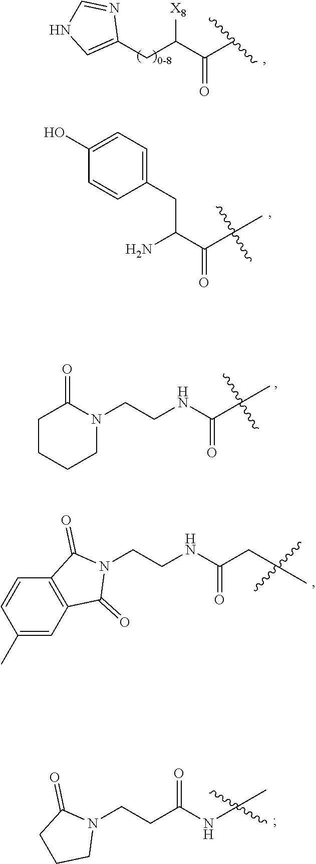

X₈ is selected from H, —OH, —NH₂, and

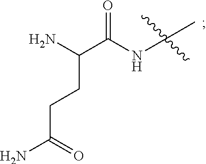 [SMILES: *NC(=O)C(N)CCC(N)=O]

Ar is selected from

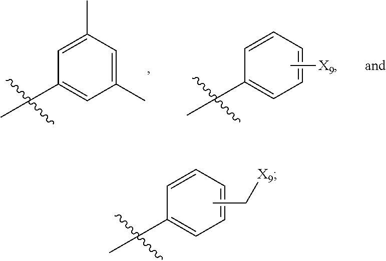

and
X₉ is selected from the group consisting of —NH₂

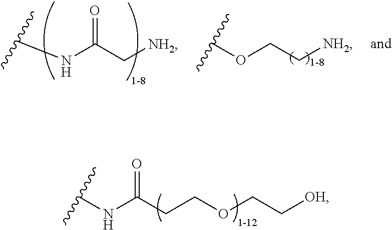

or a pharmaceutically acceptable salt thereof,
with the proviso that when X_{4b} is —OH, Ar is

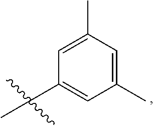 [SMILES: *c1cc(C)cc(C)c1]

### Lp is

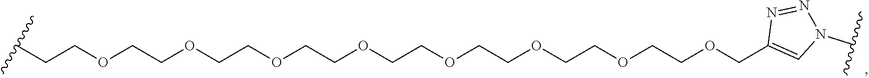

### and Q is NH₂,

X₅ is not

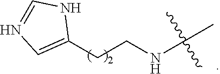

In some embodiments, the compound of Formula (LP-I) has a structure of Formula (LP-Ia):

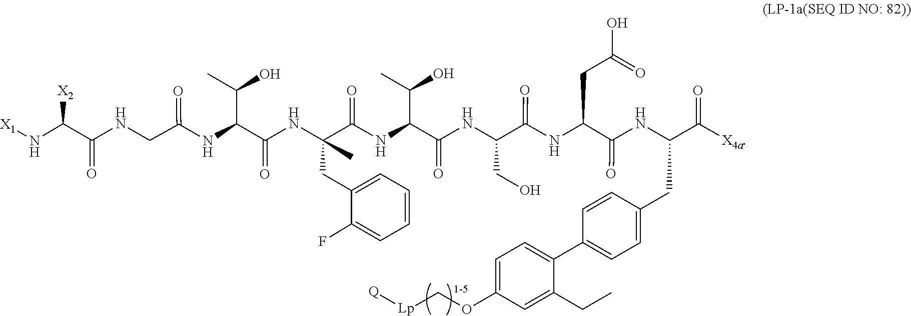

In some embodiments, Lp comprises a polyethylene glycol (PEG) segment having 1 to 36-CH₂CH₂O-(EG) units. In some embodiments, the PEG segment comprises between 2 and 30 EG units. In some embodiments, the PEG segment comprises between 4 and 24 EG units. In some embodiments, the PEG segment comprises 4 EG units, 5 EG units, 6 EG units, 7 EG units, 8 EG units, 9 EG units, 10 EG units, 11 EG units, or 12 EG units. In some embodiments, the PEG segment comprises 4 EG units. In some embodiments, the PEG segment comprises 8 EG units.

In some embodiments, the Lp comprises one or more amino acids selected from glycine, serine, glutamic acid, alanine, valine, threonine, leucine, and proline, and combinations thereof. In some embodiments, the Lp comprises 1 to 10 glycines. In some embodiments, the Lp comprises 1 to 6 serines. In some embodiments, the Lp comprises 1 to 10 glycines and 1 to 6 serines. In some embodiments, the Lp comprises 4 glycines and 1 serine.

In some embodiments, the Lp comprises a combination of a PEG segment having 1 to 36 EG units and one or more amino acids selected from glycine, serine, glutamic acid, alanine, valine, threonine, leucine, and proline, and combinations thereof.

In some embodiments, the Lp comprises a combination of a PEG segment having 1 to 36 EG units and a triazole group.

In some embodiments, the Lp comprises a combination of a PEG segment having 1 to 36 EG units; a triazole group; and —CH₂—O— group.

In some embodiments, the Lp comprises a combination of a PEG segment having 1 to 36 EG units; a triazole group; —CH₂—O— group; and —(CH₂)₁₋₂₄— group.

In some embodiments, the Lp comprises a combination of a PEG segment having 5 to 10 EG units; a triazole group; —CH₂—O— group; and —(CH₂)₁₋₅— group.

In some embodiments, the Lp is

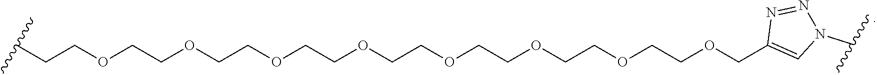

In some embodiments, X₁ is

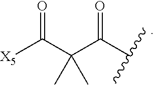

In some embodiments, X₂ is

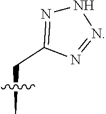

In some embodiments, X₃ is —(CH₂)₂₋₆—N₃.

In some embodiments, X₅ is

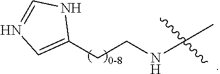

In some embodiments, X₄ₐ is —NH₂.

In some embodiments, X₄ₐ is —OH.

In some embodiments, the compound has the structure selected from the group consisting of:

| LP# | Name | Structure |
|---|---|---|
| LP1 (SEQ ID NO: 84) | DIBAC- suc- PEG4- P9 | 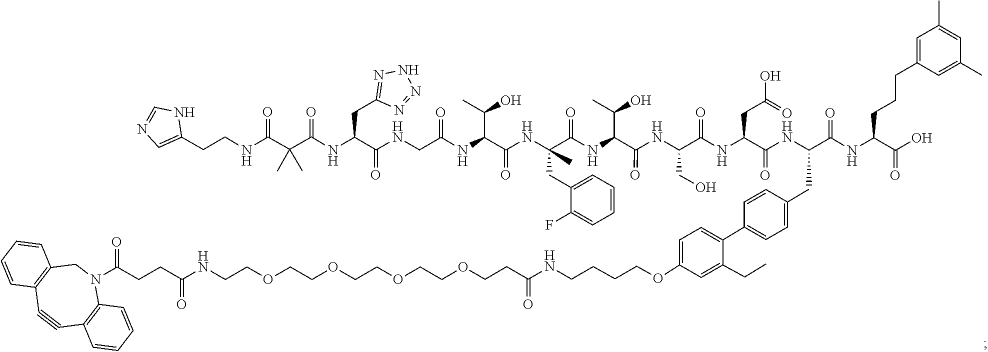 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP2 (SEQ ID NO: 85) | DIBAC- suc- PEG8- P9 | 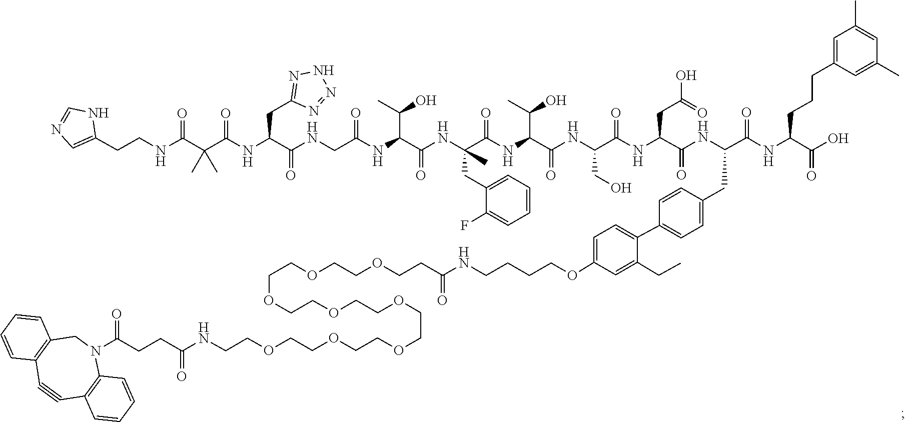 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP3 (SEQ ID NO: 86) | DIBAC- suc- PEG12- P9 | 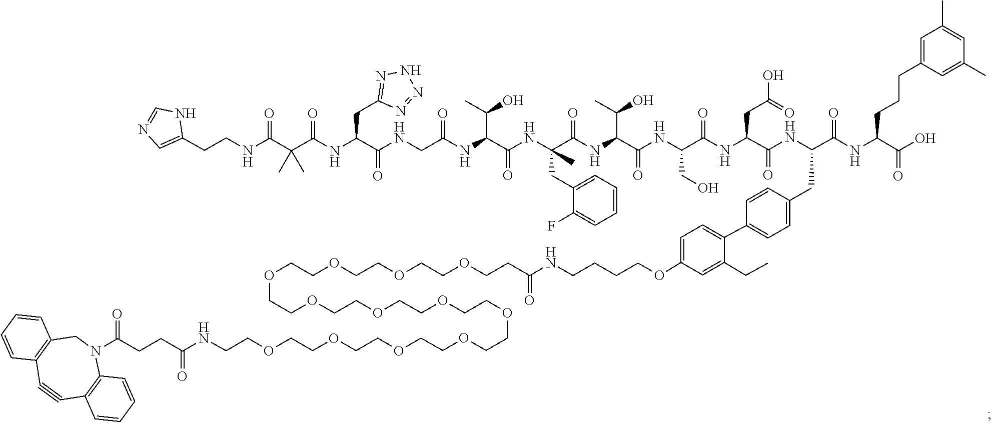 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP4 (SEQ ID NO: 87) | DIBAC- suc- PEG24- P9 | 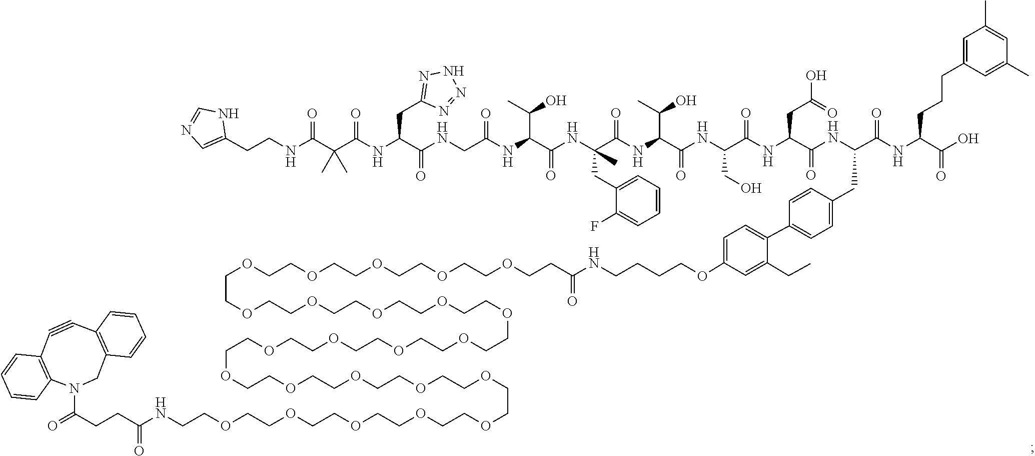 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP5 (SEQ ID NO: 88) | BCN- PEG4- carba- mate-P9 | 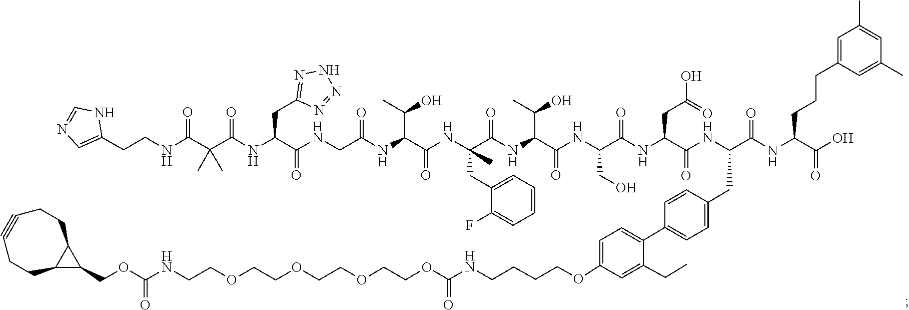 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP6 (SEQ ID NOS 89 and 90, respec- tively) | DIBAC- suc- G4S-P9 | 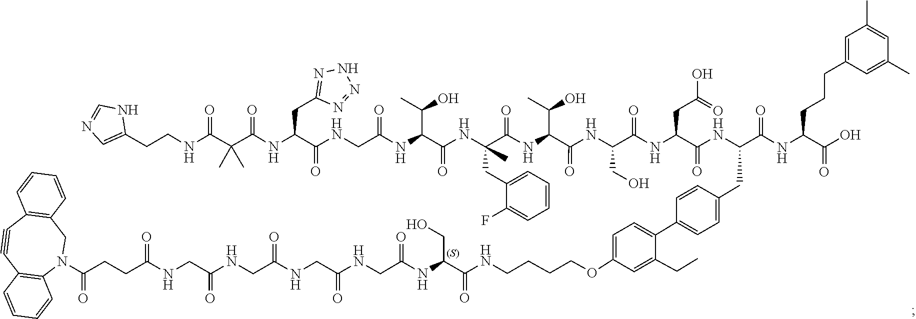 |
| LP7 (SEQ ID NOS 91 and 92, respec- tively) | DIBAC- suc- SG4-P9 | 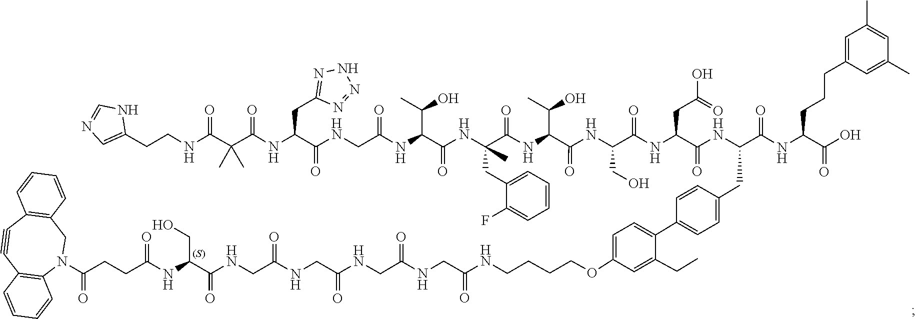 |
| LP8 (SEQ ID NO: 351) | DIBAC- suc- PEG4- triazole- P8 | 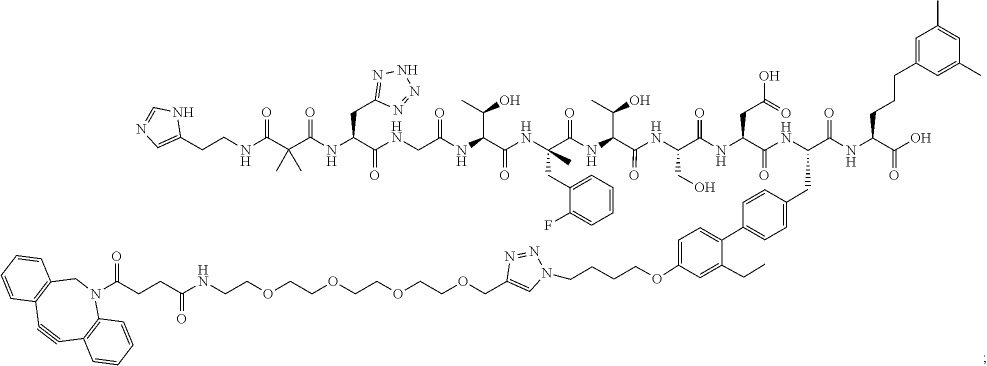 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP9 (SEQ ID NO: 93) | BCN- PEG4- triazole- P8 | 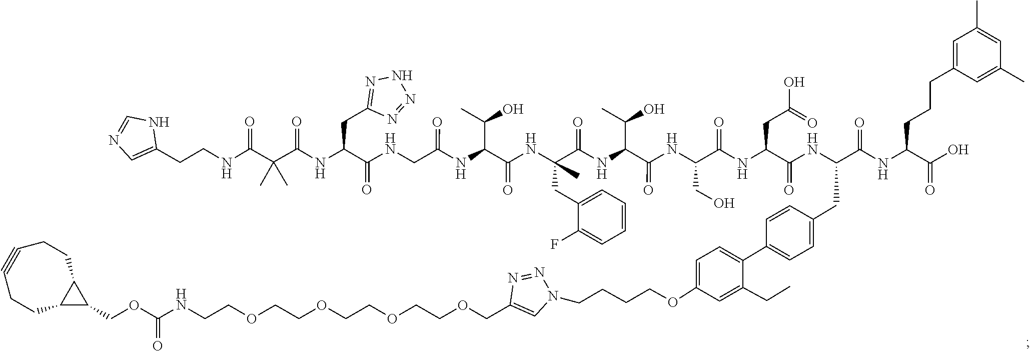 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP10 (SEQ ID NO: 94) | COT- PEG4- triazole- P8 | 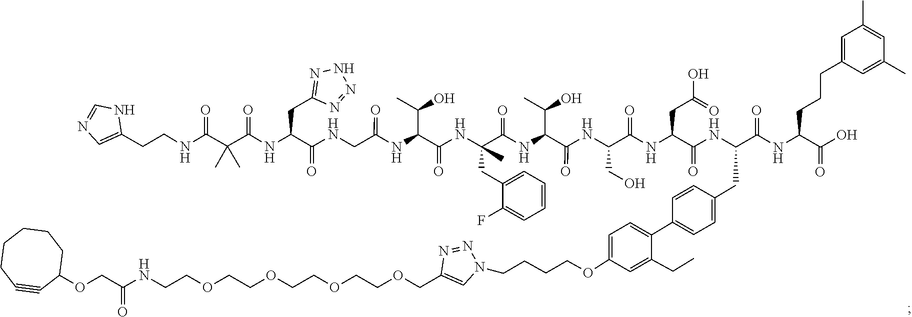 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP12 (SEQ ID NO: 95) | DIBAC- suc- PEG24- P11 | 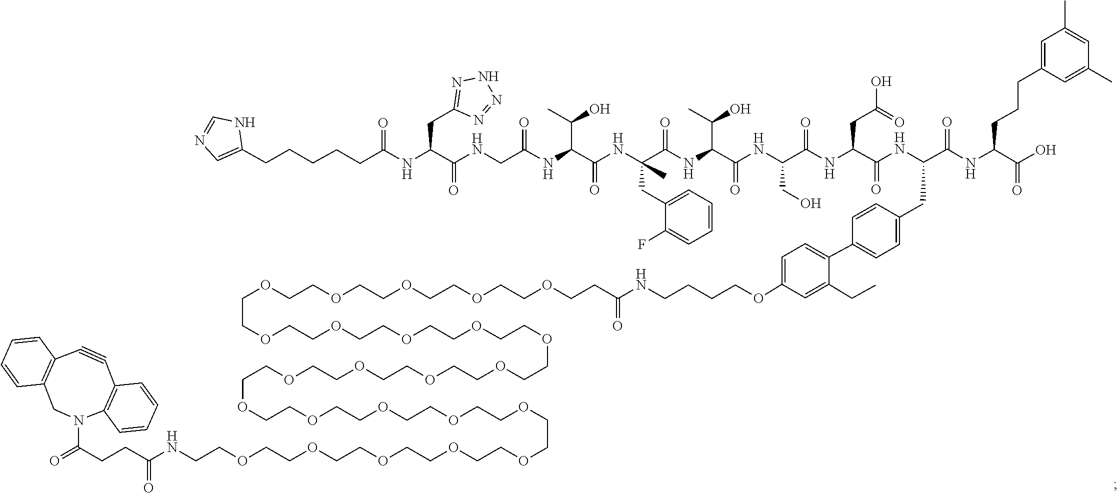 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(=O)O)cc1] |
| LP13 (SEQ ID NO: 96) | DIBAC- suc- PEG24- P4 | 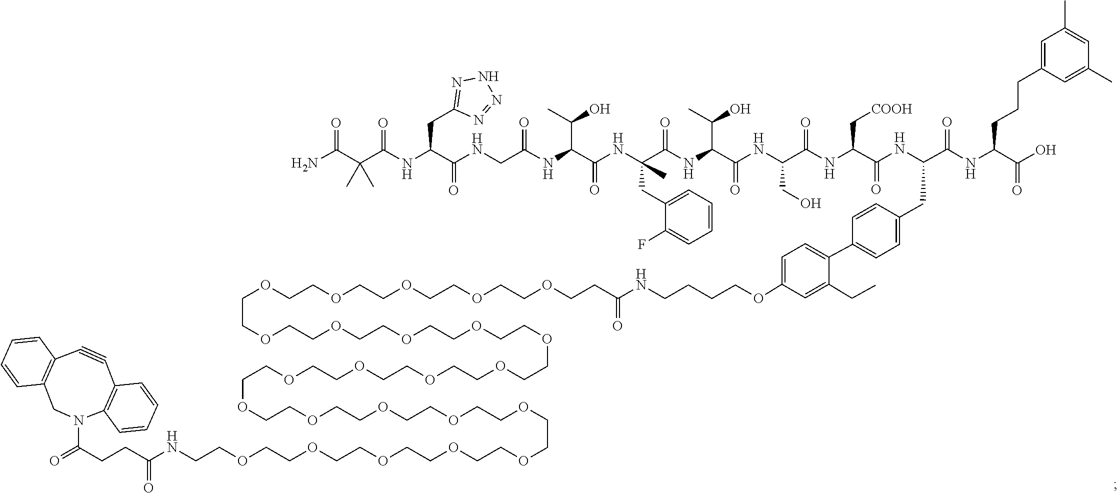 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(=O)O)cc1] |
| LP14 (SEQ ID NOS 97 and 98, respec- tively) | DIBAC- suc- G4S-P4 | 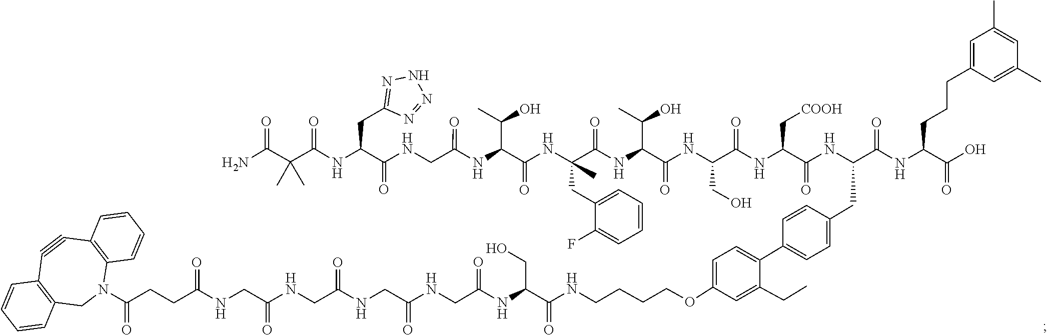 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(=O)O)cc1] |
| LP17 (SEQ ID NOS 99 and 100, respec- tively) | DIBAC- suc- G₄S- G4S-P9 | 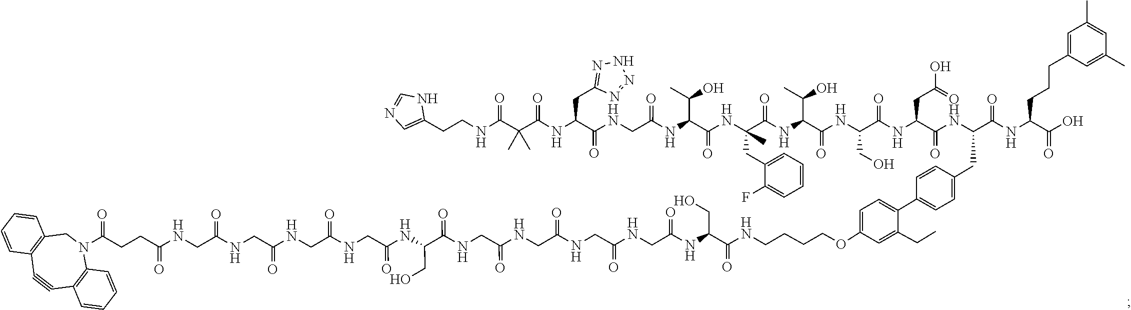 [SMILES: CCc1cc(OCCCCNC(=O)[C@H](CO)NC(=O)CNC(=O)CNC(=O)CNC(=O)CNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP18 (SEQ ID NOS 101 and 102, respec- tively) | BCN- NHC₂H₄ CO- (glucose) SG4-P9 | 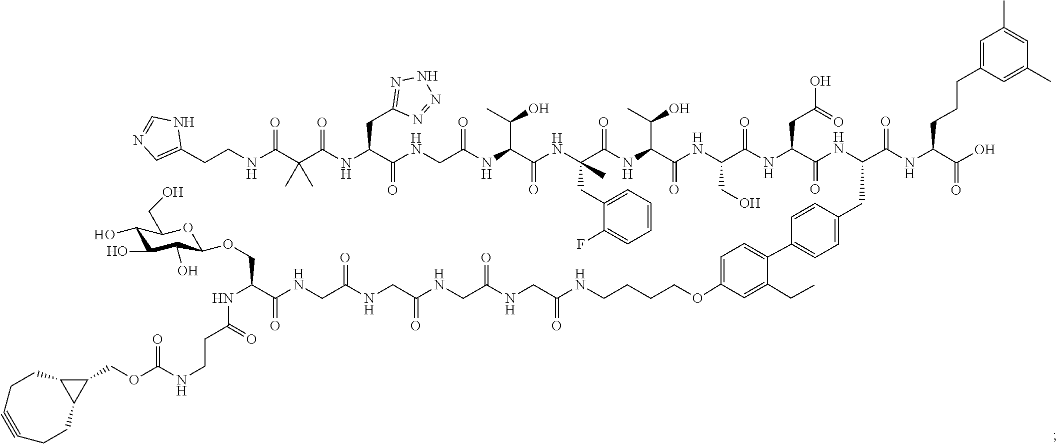 |
| LP19 (SEQ ID NOS 103 and 104, respec- tively) | COT- G₄- (R)Ser- P9 | 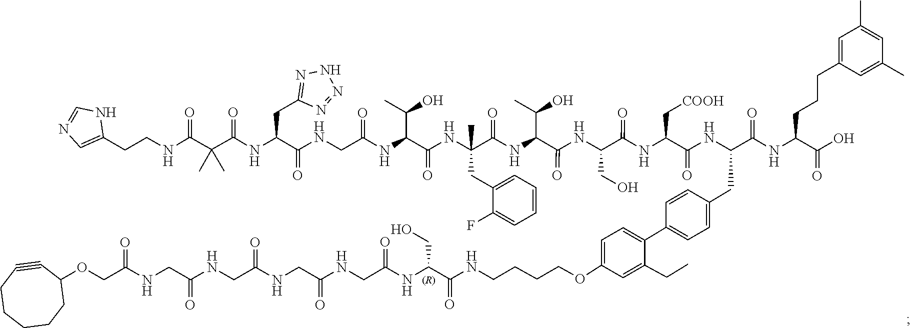 |
| LP20 (SEQ ID NOS 105 and 106, respec- tively) | DIBAC- suc- (glucose) SG4-P9 | 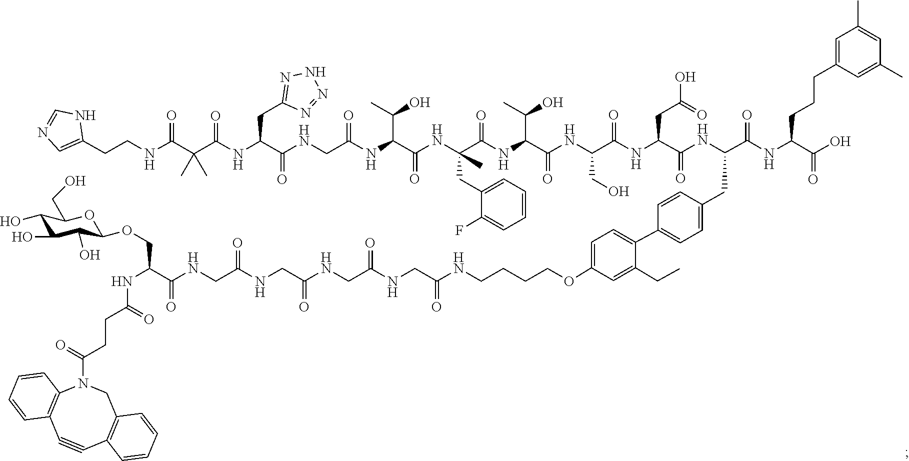 |
| LP21 (SEQ ID NO: 107) | DIBAC- suc- PEG24- P24 | 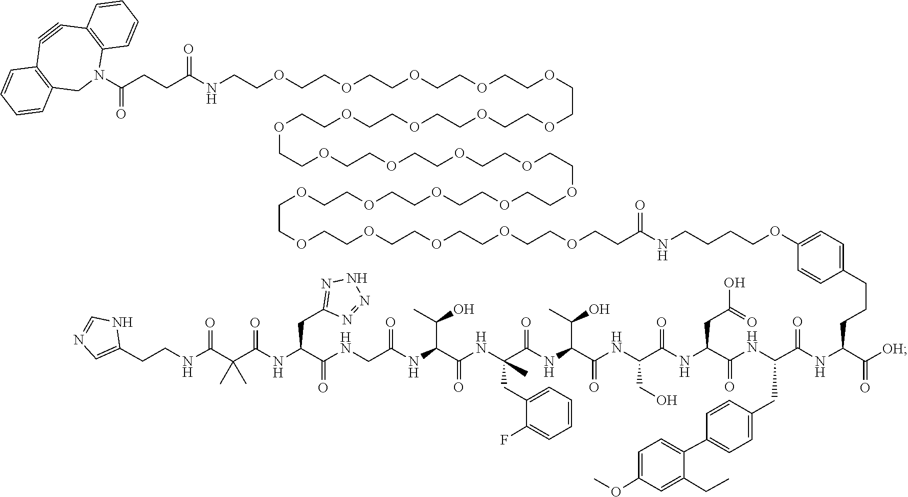 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4C#Cc4ccccc43)cc2)C(=O)O)cc1] |
| LP22 (SEQ ID NO: 108) | cyclode xtrin- triazole- DIBAC- suc- PEG24- P9 | 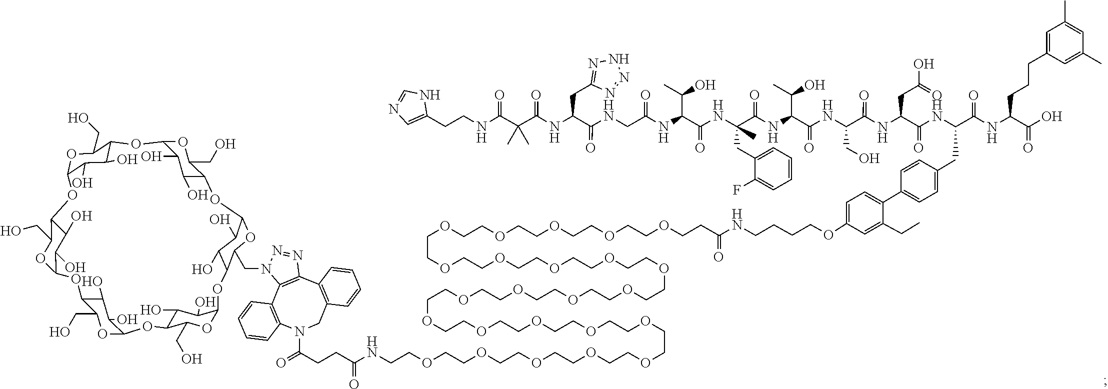 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP23 (SEQ ID NO: 109) | cyclode xtrin- triazole- DIBAC- suc- PEG24- P24 | 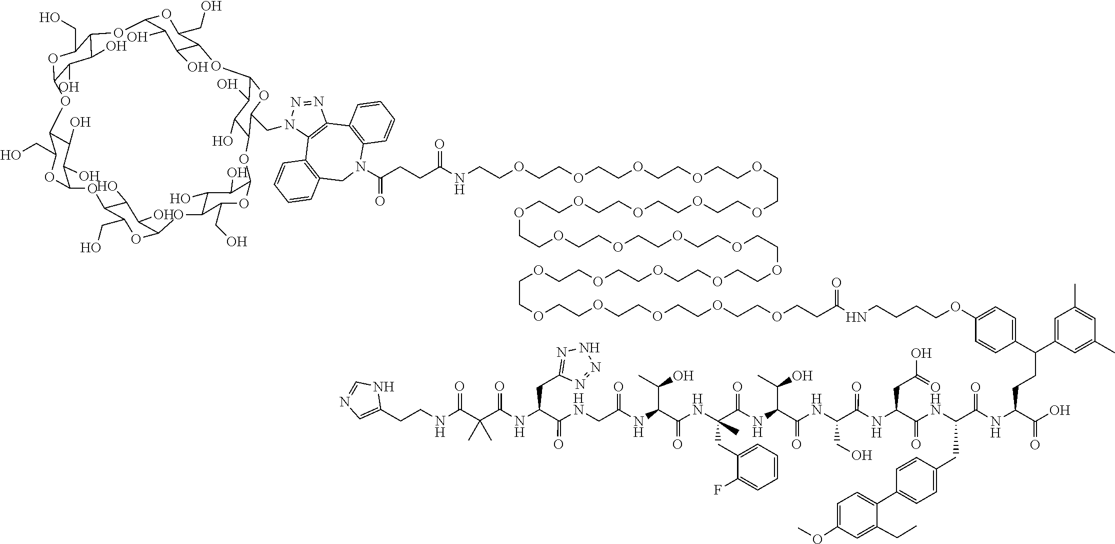 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCC(c2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4-c4c(nnn4CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)-c4ccccc43)cc2)c2cc(C)cc(C)c2)C(=O)O)cc1] |
| LP24 (SEQ ID NO: 110) | triazole- BCN- PEG4- triazole- P8 | 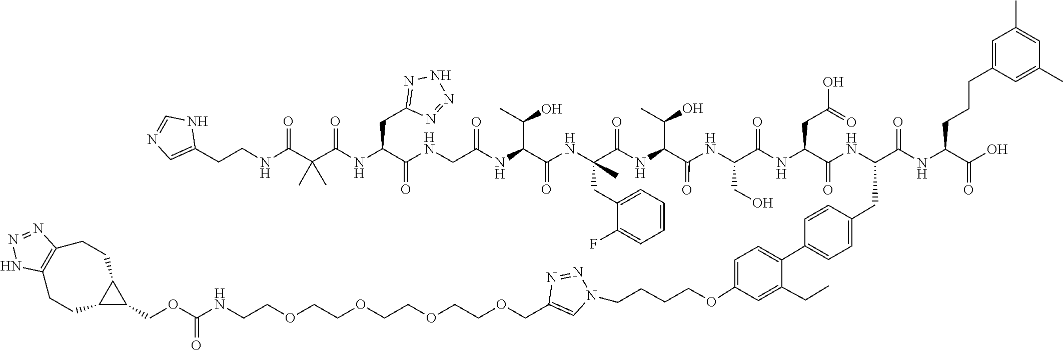 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP25 (SEQ ID NO: 111) | triazole- BCN- PEG4- carba- mate-P9 | 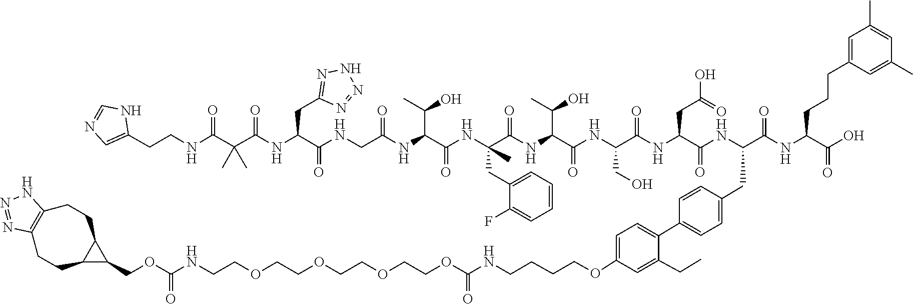 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP26 (SEQ ID NOS 112 and 113, respec- tively) | triazole- DIBAC- G4S-P9 | 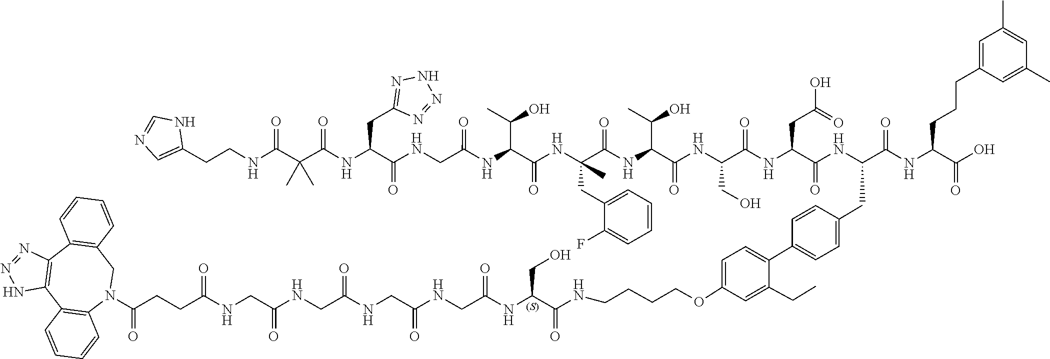 |
| LP27 (SEQ ID NO: 114) | DIBAC- suc- PEG8- triazole- P8 | 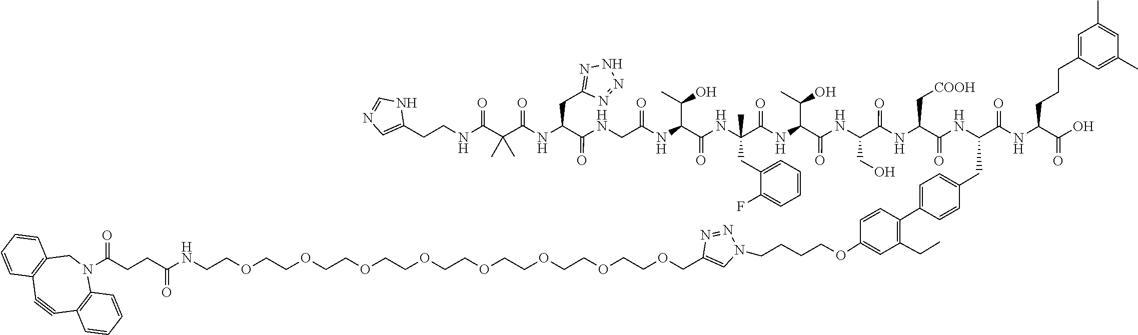 |
| LP28 (SEQ ID NO: 115) | triazole- DIBAC- suc- PEG8- triazole- P8 | 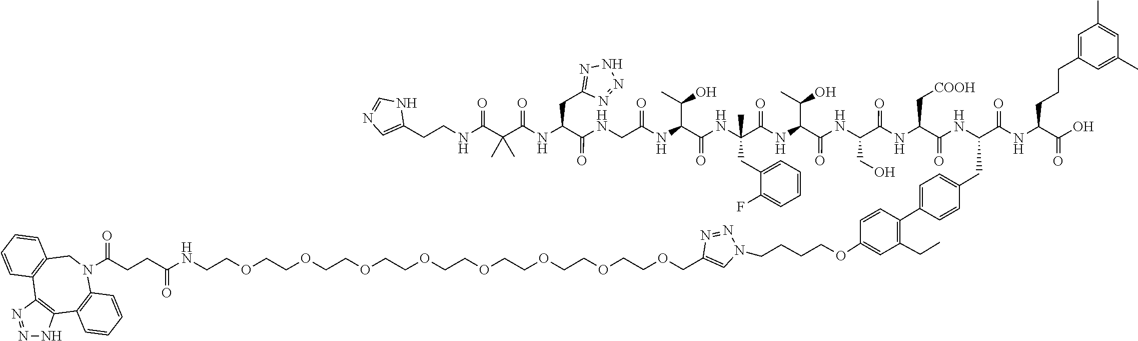 |
| LP29 (SEQ ID NO: 116) | NH2- PEG8- triazole- P19 | 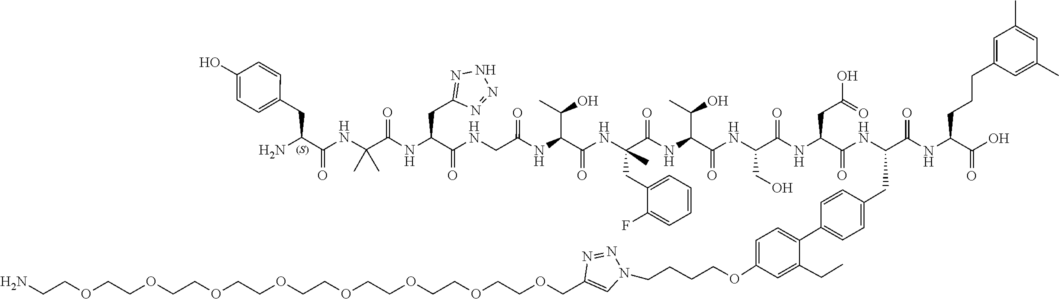 |
| LP30 (SEQ ID NO: 117) | NH2- PEG8- triazole- P35 | 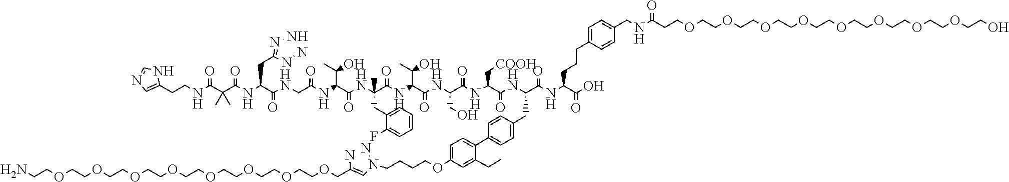 |
| LP31 (SEQ ID NO: 118) | NH2- PEG8- triazole- P8 | 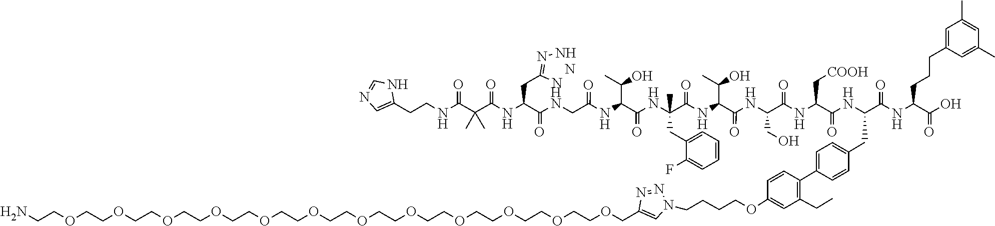 |
| LP32 (SEQ ID NO: 119) | NH2- PEG8- triazole- P8 acid | 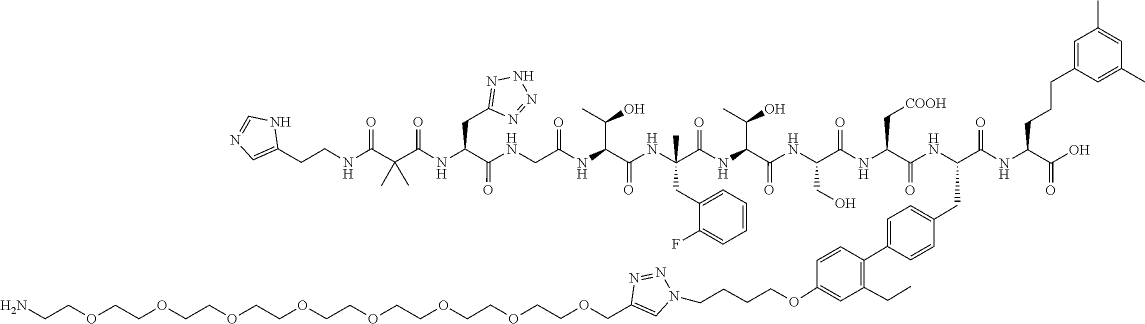 |
| LP33 (SEQ ID NO: 120) | E- PEG8- triazole- P8 | 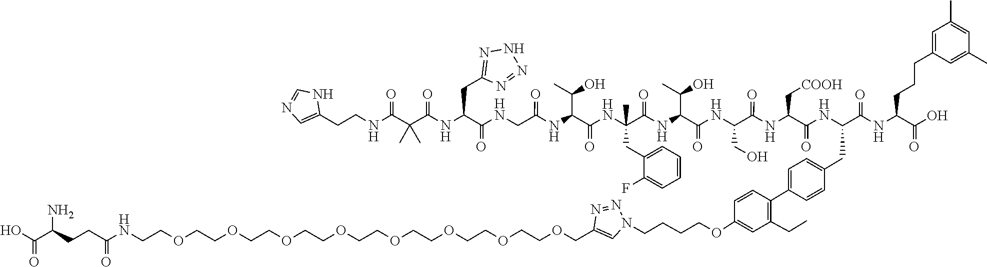 |
| LP34 (SEQ ID NOS 121 and 122, respec- tively) | GGTEP L- PEG8- triazole- P8 | 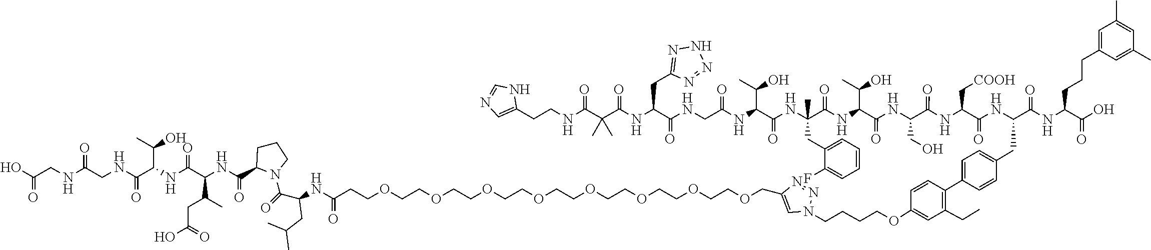 |
| LP35 (SEQ ID NOS 123 and 124, respec- tively) | Cbz- LLQGS G- PEG8- triazole- P8 | 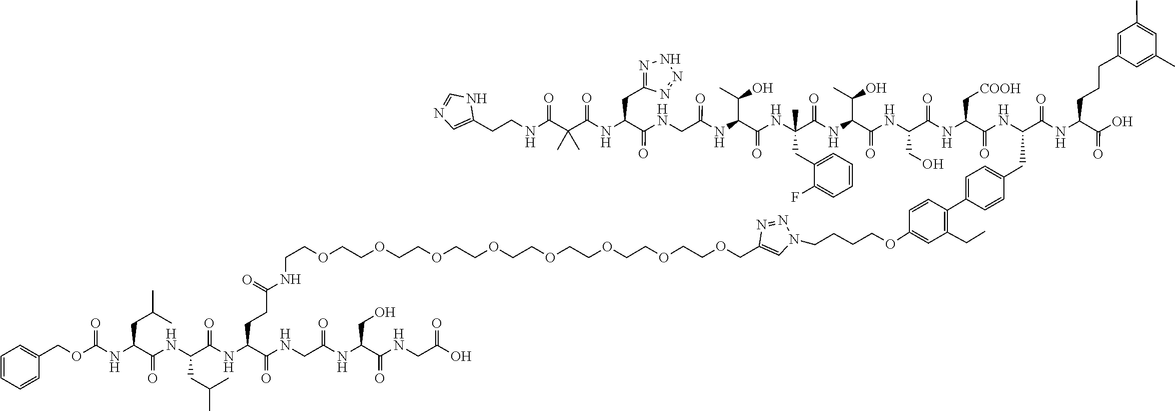 |
| LP36 (SEQ ID NOS 125 and 126, respec- tively) | G4S triazole- P40 | 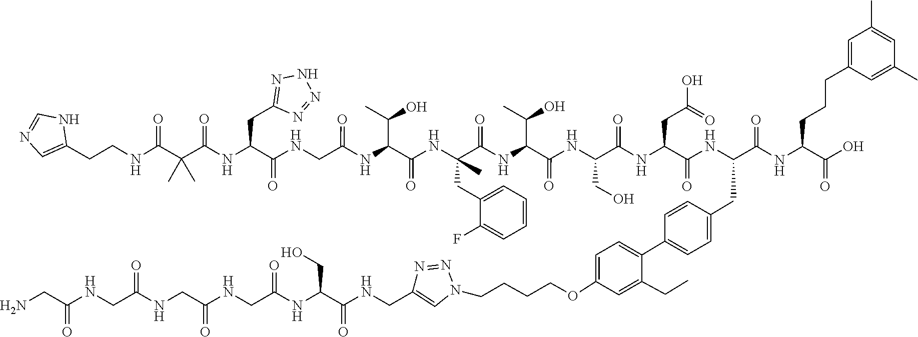 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP37 (SEQ ID NOS 127 and 128, respec- tively) | SG4- triazole- P40 | 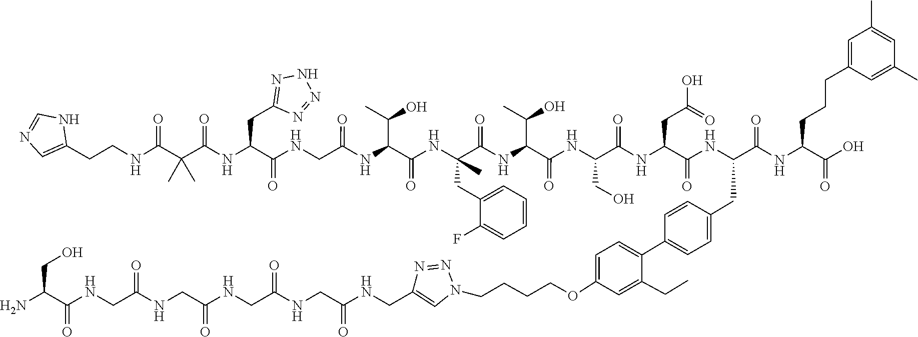 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP38 (SEQ ID NOS 129 and 130, respec- tively) | G2SG2S G2 triazole- P40 | 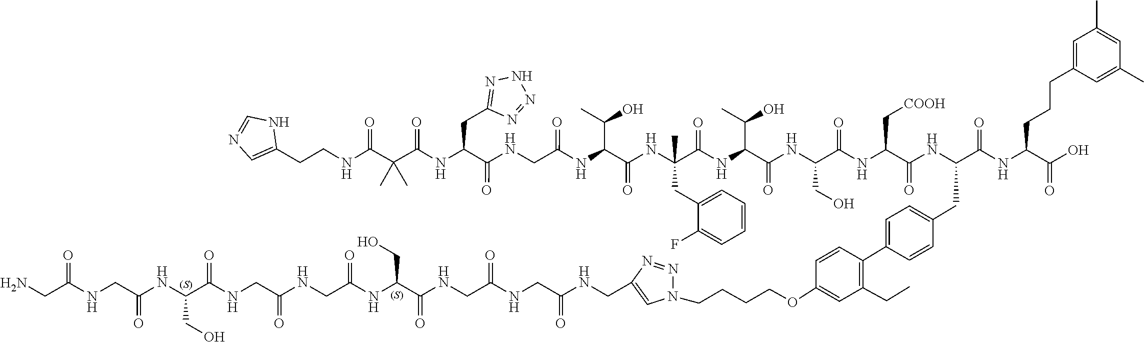 |
| LP39 (SEQ ID NOS 131 and 132, respec- tively) | G4SG4 triazole- P40 | 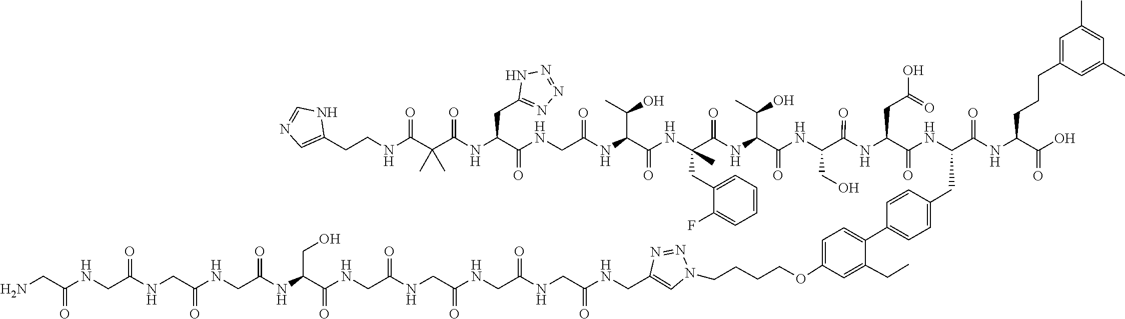 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP40 (SEQ ID NOS 133 and 134, respec- tively) | G4SG4S G4 triazole- P40 | 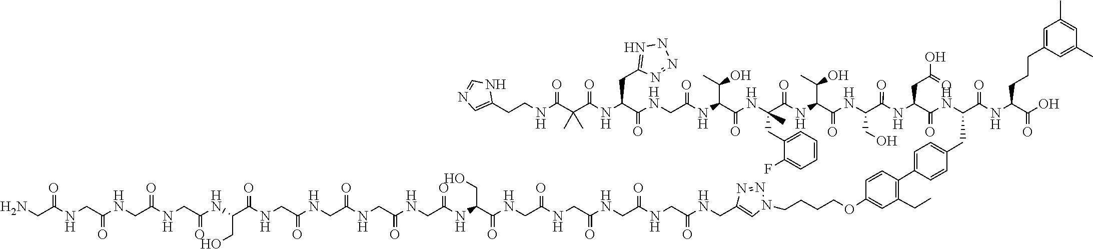 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP41 (SEQ ID NOS 135 and 136, respec- tively) | G2SG2S G2SG2 triazole- P40 | 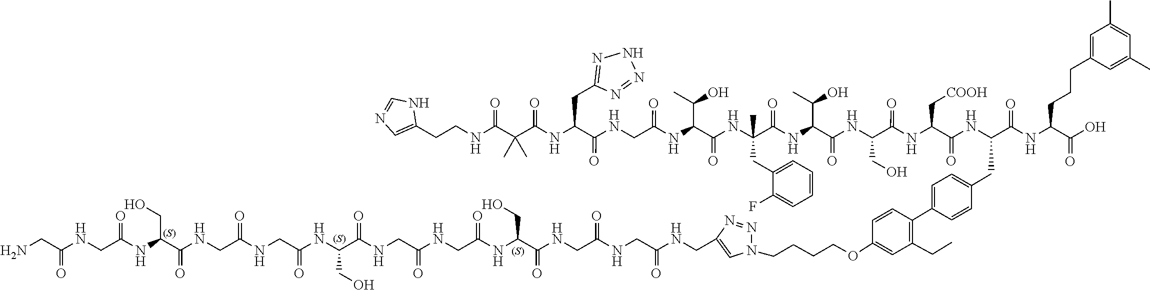 |
| LP42 (SEQ ID NOS 137 and 138, respec- tively) | C18- diacid- Glu- (AEEA) 2- G4SG4- triazole- P40 | 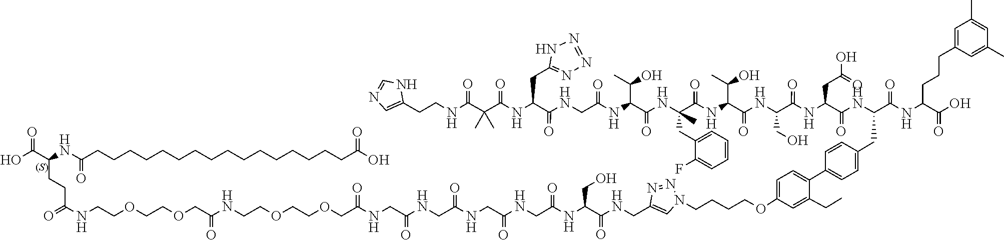 |
| LP43 (SEQ ID NOS 139 and 140, respec- tively) | C18- diacid- Glu- (AEEA) 2- G4SG4- triazole- P40 | 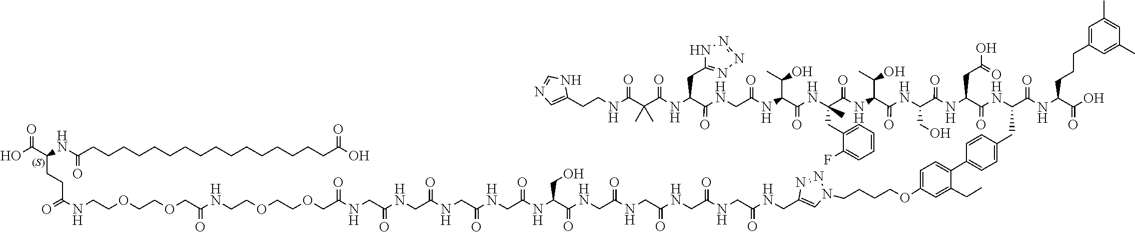 |
| LP44 (SEQ ID NO: 141) | C18- diacid- Glu- (AEEA) 2-NH2- PEG12- P40 | 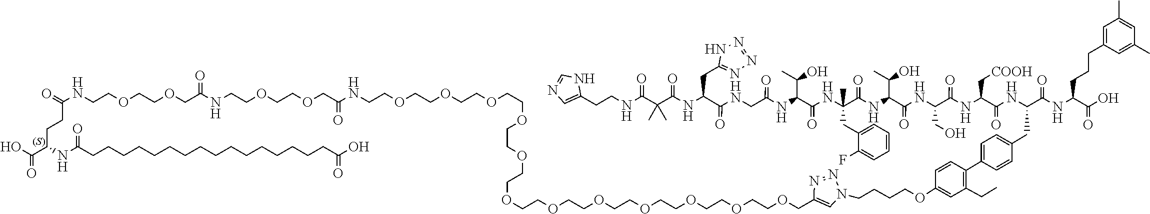 |
| LP45 (SEQ ID NO: 142) | C18- diacid- Glu- (AEEA) 2-NH2- PEG8- P35 | 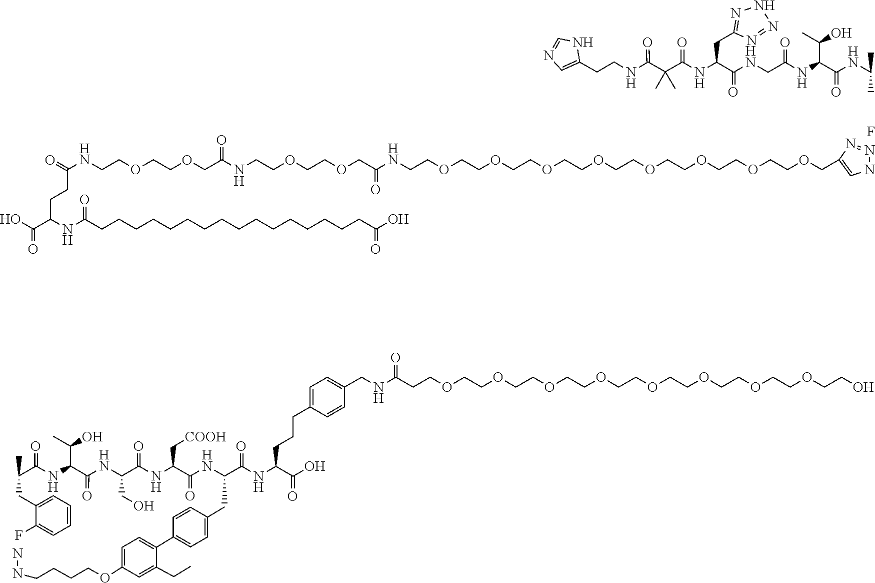 |
| LP46 (SEQ ID NO: 143) | Leu- Leu- Glu- PEG8- P40 | 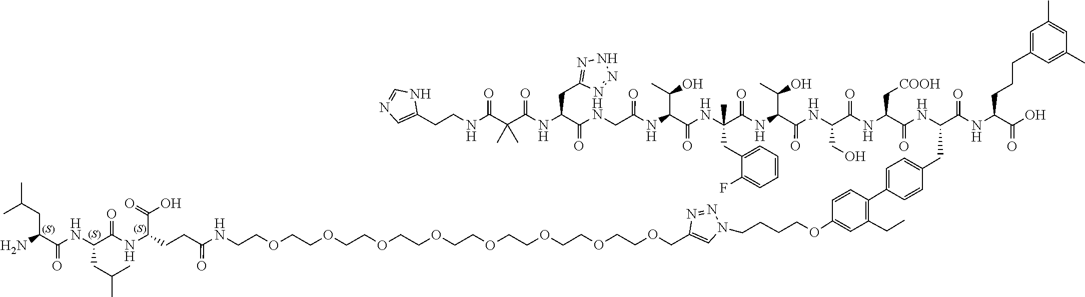 |
| LP47 (SEQ ID NO: 144) | M6447 series | 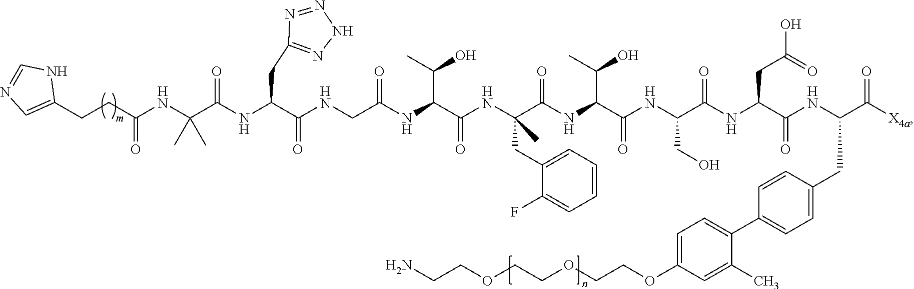 |
| | | wherein X₄ₐ is —OH or —NH₂; m is an integer of one to seven; n is an integer of one |
| | | to twelve; |
| LP48 (SEQ ID NO: 145) | M6447 series | 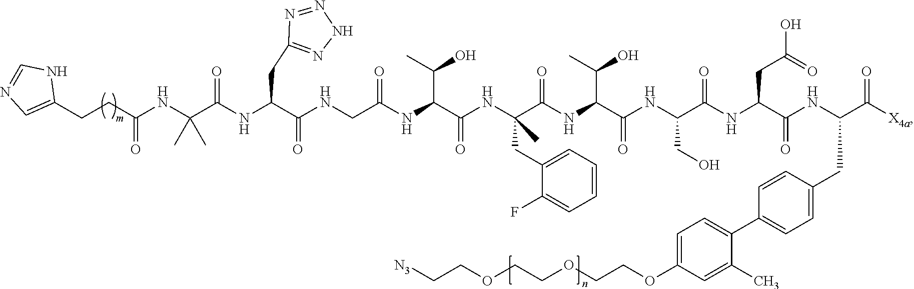 |
| | | wherein X₄ₐ is —OH or —NH₂; m is an integer of one to seven; n is an integer of one |
| | | to twelve; |

or a pharmaceutically acceptable salt thereof.

In another aspect, provided herein is an antibody or an antigen-binding fragment thereof conjugated to the compound of any of the embodiments described herein directly or through a non-cleavable linker.

In another aspect, provided herein is an antibody or an antigen-binding fragment thereof conjugated to the compound of any of the embodiments described herein.

In another aspect, provided herein is a compound having a structure of Formula (A):

BA-(L-P)ₘ (A),
wherein:
BA is an antibody or an antigen-binding fragment thereof,
L is a non-cleavable linker;
P is a payload having the structure selected from the group consisting of:

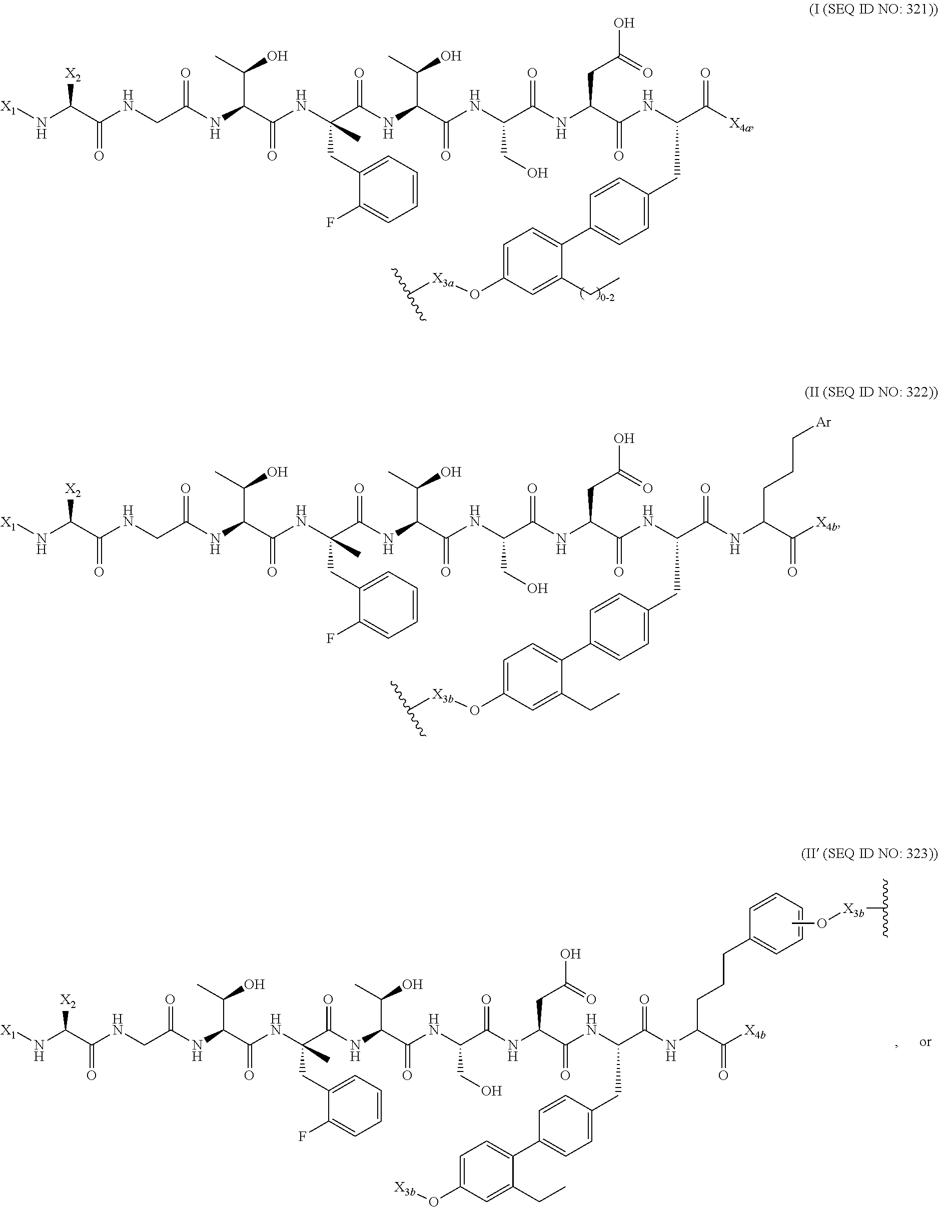

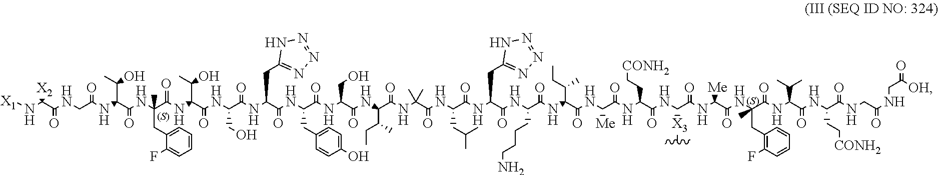

wherein

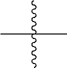

is the point of attachment of the payload to L;
X₁ is selected from H;

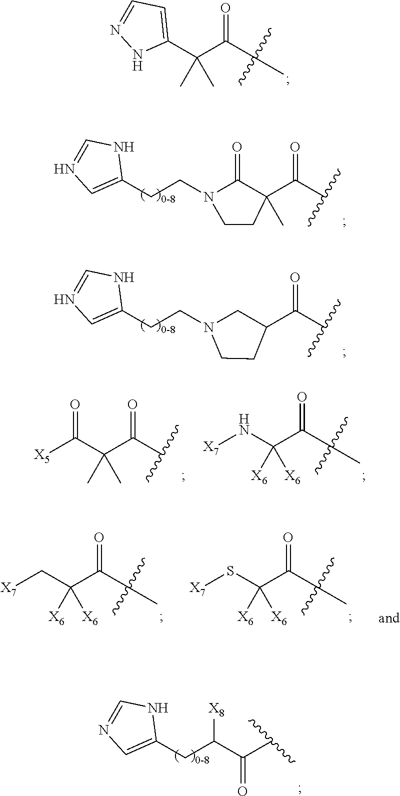

X₂ is selected from

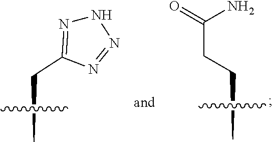

X₃ₐ is selected from bond, —CH₃, —(CH₂)₁₋₄(OCH₂CH₂)₂₋₁₅—NH₂, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)—NH—; X_{3b} is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₆—NH—;
Tr is a triazole moiety;
X₄ₐ is —NH₂ or —OH;
X_{4b} is —OH;
X₅ is selected from —OH, —NH₂, —NH—OH, and

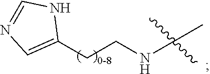

X₃ is independently at each occurrence selected from H, —OH, —CH₃, and —CH₂OH;
X₇ is selected from H,

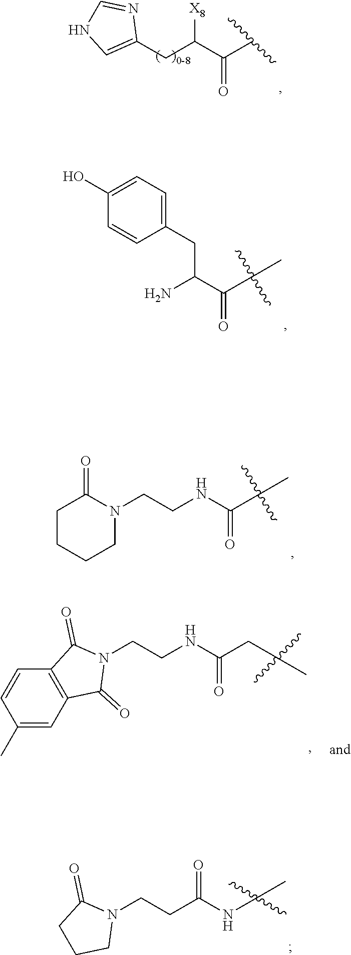

X₈ is selected from H, —OH, —NH₂, and

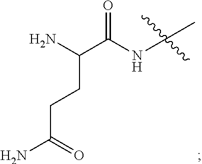 [SMILES: *NC(=O)C(N)CCC(N)=O]

Ar is selected from

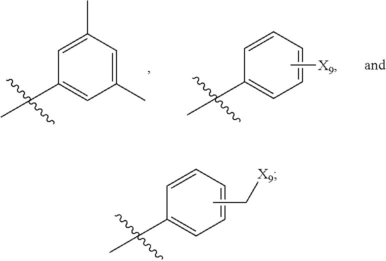

and
X₉ is selected from the group consisting of —NH₂,

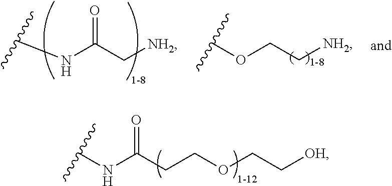

or a pharmaceutically acceptable salt thereof,
with the proviso that when with the proviso that when X_{4b} is —OH, Ar is

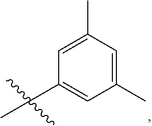 [SMILES: *c1cc(C)cc(C)c1]

### and L is

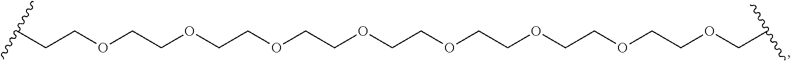 [SMILES: *CCOCCOCCOCCOCCOCCOCCOCCOC*]

### X₅ is not

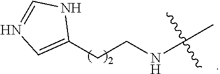

In another aspect, provided herein is a compound having a structure of Formula (B):

BA-L-P (B),
wherein:
BA is an antibody or an antigen-binding fragment thereof,
L is a non-cleavable linker;
P is a payload having the structure selected from the group consisting of:

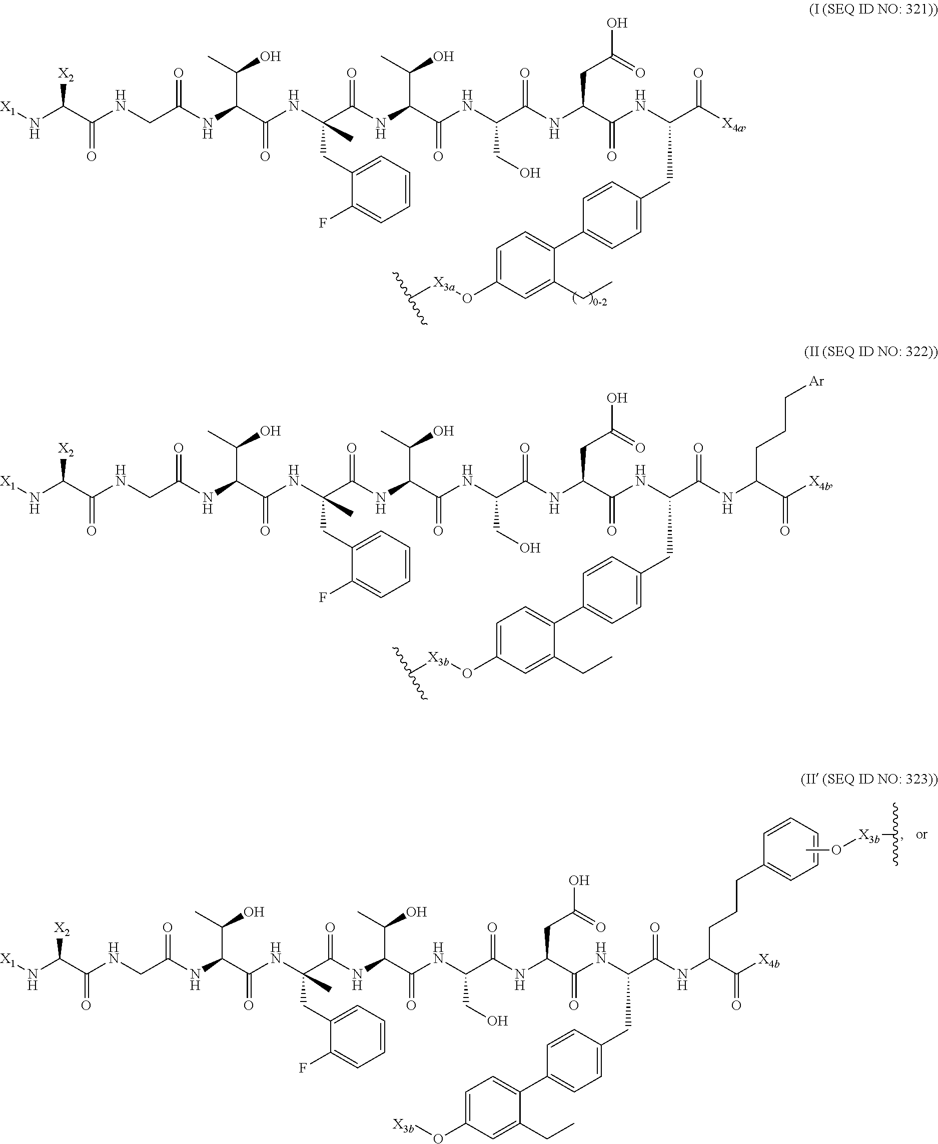

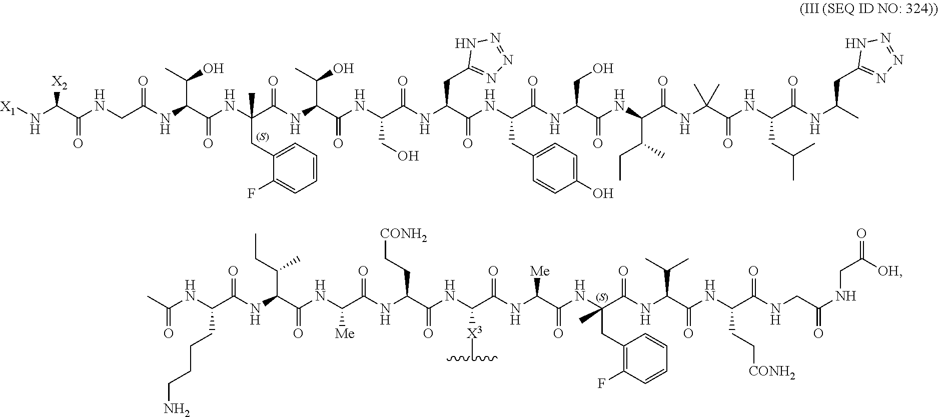

wherein

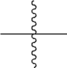

is the point of attachment of the payload to L;
X₁ is selected from H;

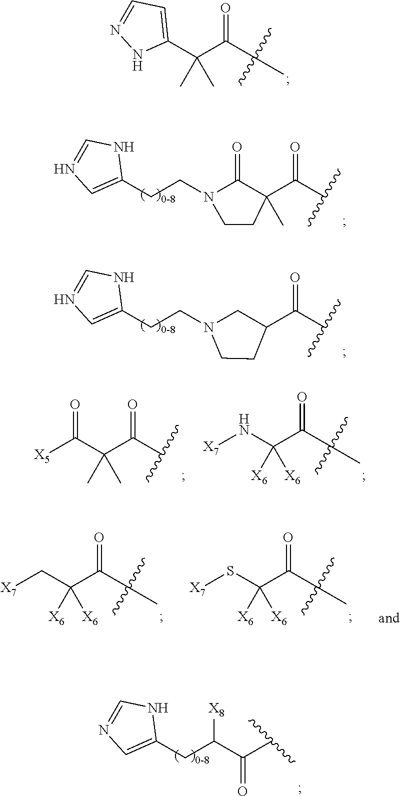

X₂ is selected from

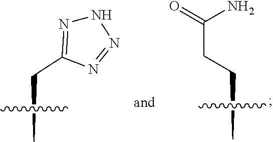

X₃ₐ is selected from bond, —CH₃, —(CH₂)₁₋₄(OCH₂CH₂)₂₋₁₅—NH₂, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH₂—, —(CH₂)₂₋₆-Tr-, and —(CH₂)₂₋₆-Tr-(CH₂)₁₋₆—NH—; X_{3b} is selected from bond, —CH₃, —(CH₂)₂₋₆—NH₂, —(CH₂)₂₋₆—N₃, —(CH₂)₂₋₆-Tr-(CH₂)—NH₂, —(CH₂)₂₋₆—NH—, —(CH₂)₂₋₆-Tr-, and —(CH₂)₂₋₆-Tr-(CH₂)₆—NH—;
Tr is a triazole moiety;
X₄ₐ is-NH₂ or —OH;
X_{4b} is —OH;
X₅ is selected from —OH, —NH₂, —NH—OH, and

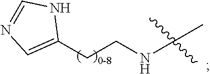

X₃ is independently at each occurrence selected from H, —OH, —CH₃, and —CH₂OH;
X₇ is selected from H,

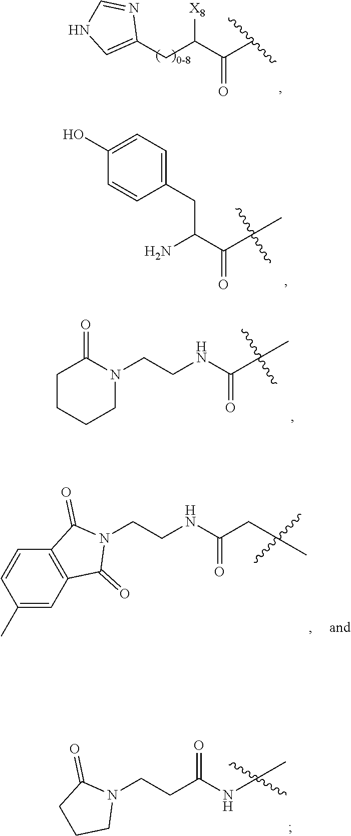

X₈ is selected from H, —OH, —NH₂, and

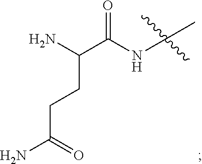 [SMILES: *NC(=O)C(N)CCC(N)=O]

Ar is selected from

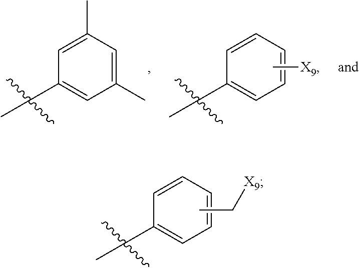

and
X₉ is selected from the group consisting of —NH₂,

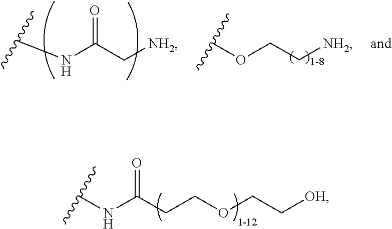

or a pharmaceutically acceptable salt thereof,
with the proviso that when with the proviso that when X_{4b} is —OH, Ar is

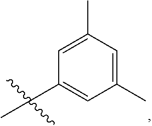 [SMILES: *c1cc(C)cc(C)c1]

### and L is

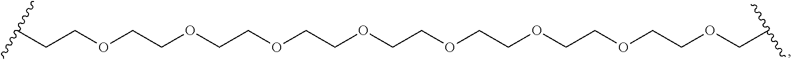 [SMILES: *CCOCCOCCOCCOCCOCCOCCOCCOC*]

### X₅ is not

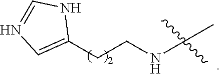

In some embodiments, P has a structure of Formula (Ia):

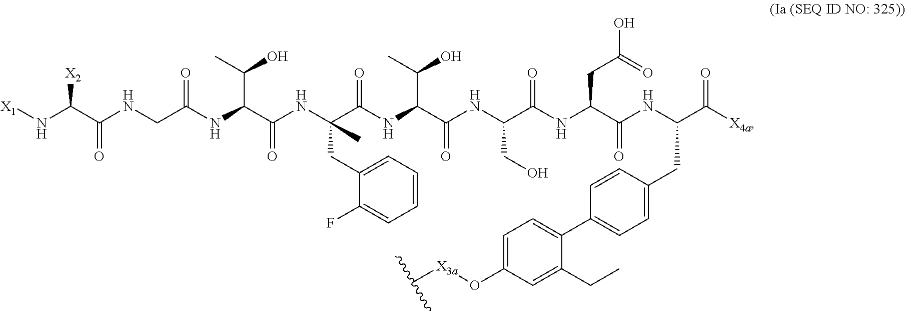

wherein
X₃ₐ is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—.

In some embodiments of the compound described herein, BA is a glucagon-like peptide-1 receptor (GLP1R)-targeting antibody or an antigen-binding fragment thereof. In some embodiments, the GLP1R-targeting antibody is a GLP1R agonist antibody. In some embodiments, the GLP1R-targeting antibody is 5A10, 9A10, AB9433-I, h38C2, PA5-111834, NLS1205, MAB2814, EPR21819, or glutazumab.

In some embodiments, BA is a glucagon-like peptide-1 receptor (GLP1R)-targeting antibody or an antigen-binding fragment thereof comprising REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280.

In some embodiments, the anti-GLP1R antibodies and antigen-binding fragments of the present invention are glutaminyl-modified. The term “glutaminyl-modified” antibody, for example, refers to an antibody (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) modified with a payload or a linker-payload (LP) as described in this disclosure, wherein the conjugation of the payload or linker-payload is through at least one covalent linkage from a glutamine side chain of said antibody or a glutamine-Tag (Q-tag)) to a primary amine compound (e.g., a Payload or Linker-Payload) of the present disclosure.

In particular embodiments, the primary amine compound is linked through an amide linkage on the glutamine side chain. In certain embodiments, the glutamine is an endogenous glutamine. In certain embodiments, the glutamine residue is naturally present in a CH2 or CH3 domain of the BA. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags or TGase recognition tag).

In some embodiments, the conjugate compound (ADC, ATL, or ADTL) has a structure (SEQ ID NOS 326-329 disclosed below, respectively, in order of appearance).

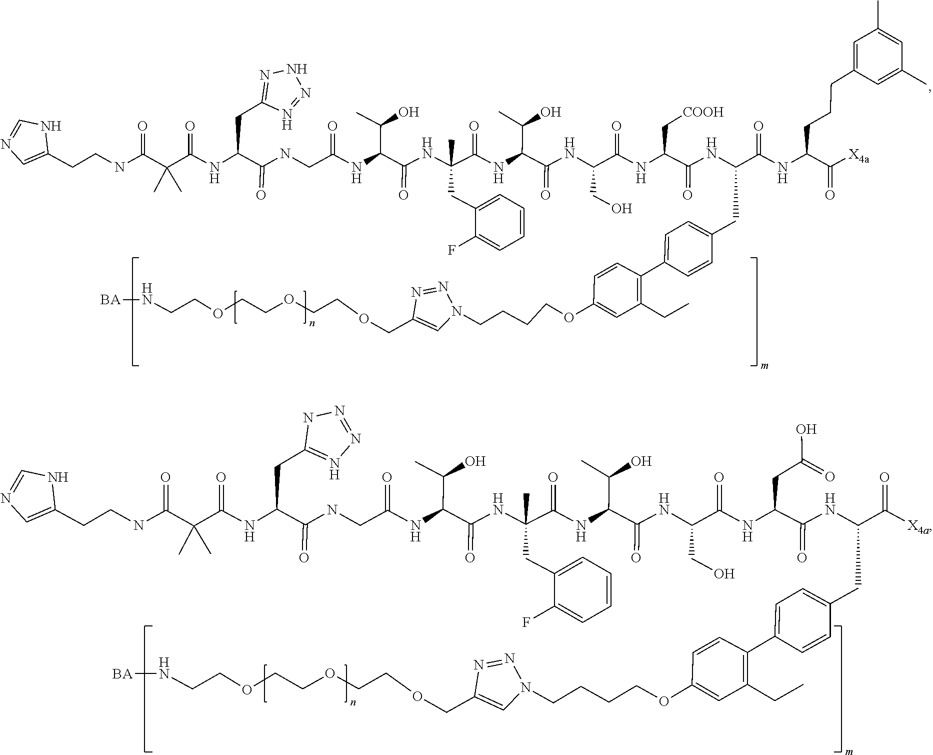

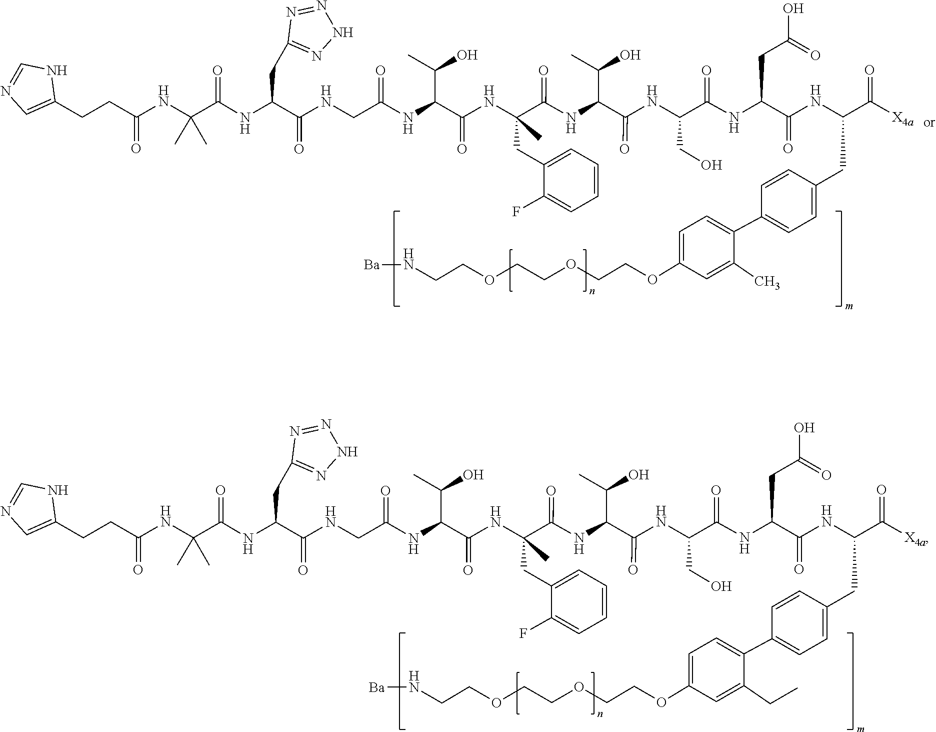

wherein X₄ₐ is —NH₂ or —OH; m ranges from about one to ten; m is an integer of one to twelve; and BA is a GLP1R antibody or an antigen binding fragment thereof. In some embodiments, m ranges from about 1 to 4, and n is six. In some other embodiments, X₄ₐ is —NH₂. In some other embodiments, X₄ₐ is-OH. Yet in some other embodiments, BA is a GLP1R antibody selected from the group consisting of REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; and REGN9280.

In some embodiments, the linker L is attached to one or both heavy chains of the BA. In some embodiments, the linker L is attached to one or both heavy chain variable domains of the BA. In some embodiments, the linker L is attached to one or both light chains of the BA. In some embodiments, the linker L is attached to one or both light chain variable domains of the BA.

In some embodiments, the linker L is attached to BA via a glutamine residue. In some embodiments, the glutamine residue is introduced to the N-terminus of one or both heavy chains of the BA. In some embodiments, the glutamine residue is introduced to the N-terminus of one or both light chains of the BA. In some embodiments, the glutamine residue is naturally present in a CH2 or CH3 domain of the BA. In some embodiments, the glutamine residue is introduced to the BA by modifying one or more amino acids. In some embodiments, the glutamine residue is Q295 or N297Q.

In some embodiments, the linker L is attached to BA via a lysine residue. In some other embodiments, the linker L is formed via Click Chemistry. Yet in some other embodiments, the linker L is formed via multiple steps of a process comprising Click Chemistry, transglutaminase-assisted conjugation, and chemo-selective nuleiphilic reaction through the side chain thiol group of a cysteine residue or the side chain primary amine of a lysine residue of BA.

In some embodiments, the antibody or antigen-binding fragment thereof is aglycosylated. In some embodiments, the antibody or antigen-binding fragment thereof is deglycosylated. In some embodiments, the antigen-binding fragment is an Fab fragment.

In one embodiment, m is 1. In one embodiment, m is an integer from 2 to 4. In one embodiment, m is 2.

In some embodiments, the linker L has the structure of formula (L′):

-La-Y-Lp- (L′),
wherein La is a first linker covalently attached to the BA;
Y is a group comprising a triazole, and
Lp is absent or a second linker covalently attached to the P, wherein when Lp is absent, Y is also absent.

In some embodiments, Y-Lp is absent.

In some embodiments, Y has a structure selected from the group consisting of:

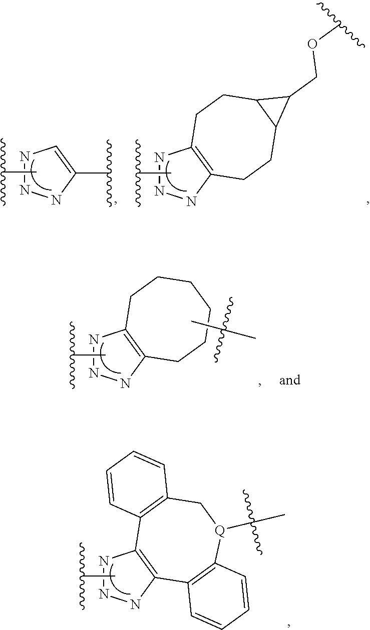

wherein Q is C or N.

In some embodiments, Lp comprises a polyethylene glycol (PEG) segment having 1 to 36-CH₂CH₂O-(EG) units. In some embodiments, the PEG segment comprises between 2 and 30 EG units. In some embodiments, the PEG segment comprises between 4 and 24 EG units. In some embodiments, the PEG segment comprises 4 EG units, or 8 EG units, or 12 EG units, or 24 EG units.

In some embodiments, Y-Lp has a structure selected from the group consisting of:

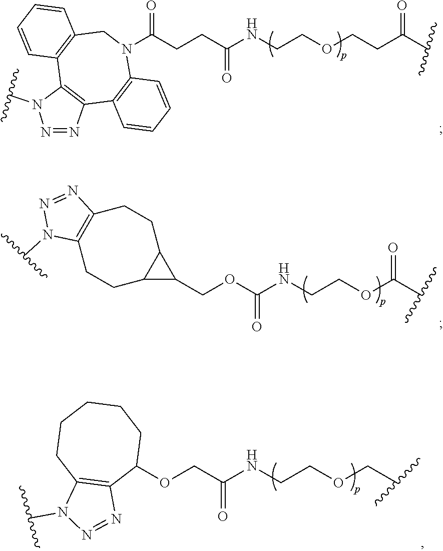

or a triazole regioisomer thereof,
wherein p is an integer from 1 to 36.

In some embodiments, the Lp comprises one or more amino acids selected from glycine, serine, glutamic acid, alanine, valine, and proline and combinations thereof. In some embodiments, the Lp comprises 1 to 10 glycines. In some embodiments, the Lp comprises 1 to 6 serines. In some embodiments, the Lp comprises 1 to 10 glycines and 1 to 6 serines. In some embodiments, the Lp comprises 4 glycines and 1 serine. In some embodiments, the Lp is selected from the group consisting of Gly-Gly-Gly-Gly-Ser (G₄S) (SEQ ID NO: 1), Ser-Gly-Gly-Gly-Gly (SG₄) (SEQ ID NO: 2), and Gly-Gly-Gly-Gly-Ser-Gly-Gly-Gly-Gly-Ser (G₄S-G₄S) (SEQ ID NO: 3).

In some embodiments, the Lp comprises a combination of a PEG segment having 1 to 36 EG units and one or more amino acids selected from glycine, serine, glutamic acid, alanine, valine, and proline and combinations thereof. In some embodiments, the serine residue comprises a carbohydrate group. In some embodiments, the serine residue comprises a glucose group.

In some embodiments, Lp has a structure selected from the group consisting of:

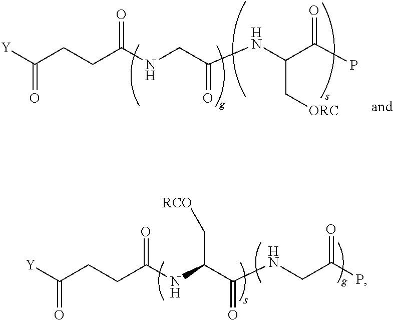

wherein Y is the group comprising a triazole and P is the payload, and wherein Rc is selected from H and glucose, g is an integer from 1 to 10 and s is an integer from 0 to 4.

In some embodiments, Y-Lp has a structure selected from the group consisting of (SEQ ID NOS: 330-335 disclosed below, respectively, in order of appearance):

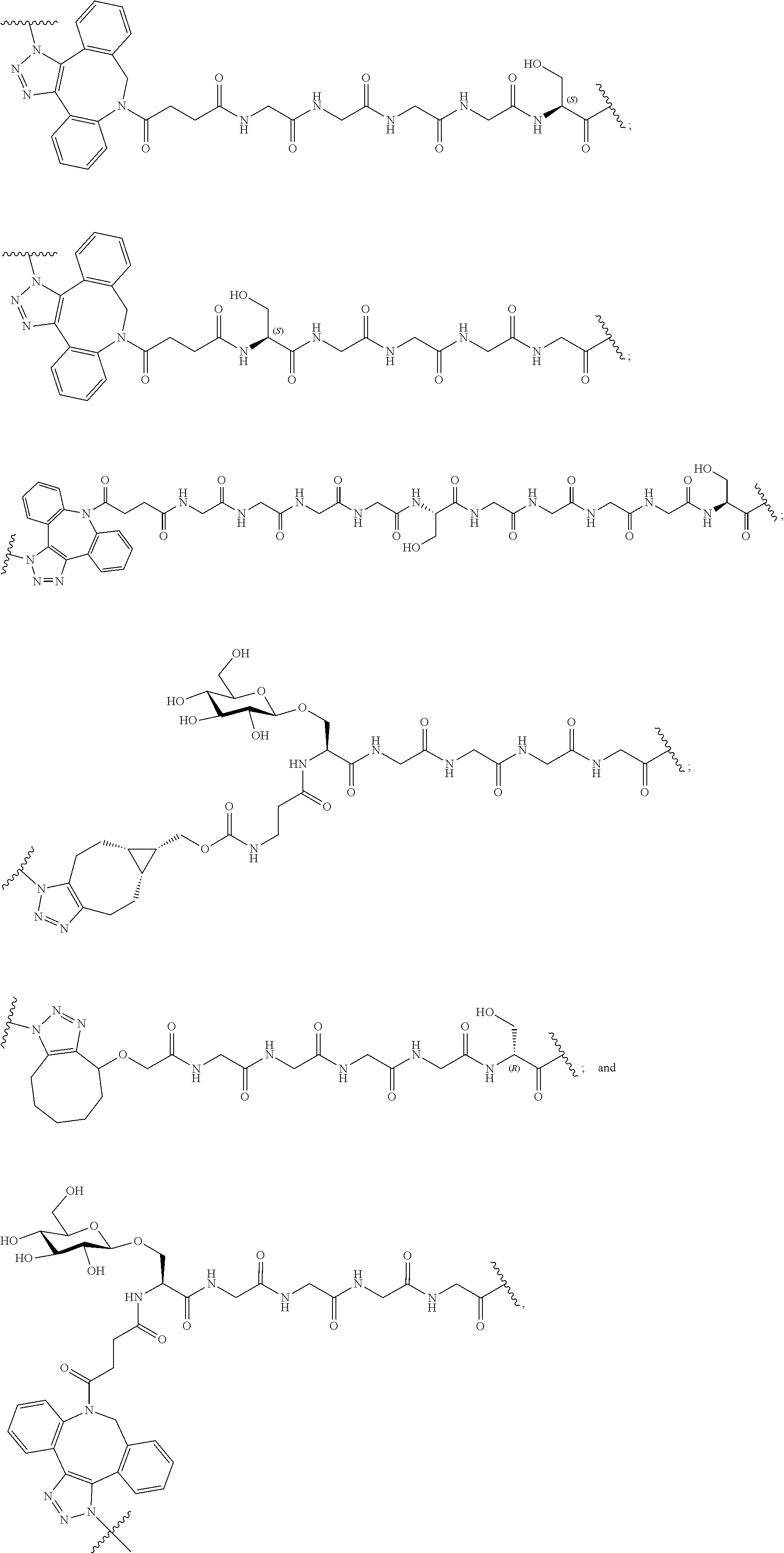

or a triazole regioisomer thereof.

In some embodiments, La comprises a polyethylene glycol (PEG) segment having 1 to 36-CH₂CH₂O-(EG) units. In some embodiments, the PEG segment comprises 4 EG units, or 8 EG units, or 12 EG units, or 24 EG units. In some embodiments, the PEG segment comprises 8 EG units.

In some embodiments, La has a structure selected from the group consisting of:

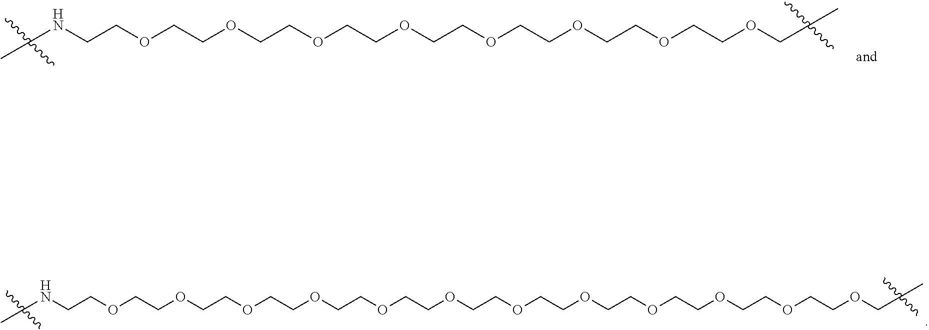 [SMILES: *COCCOCCOCCOCCOCCOCCOCCOCCN* ; *COCCOCCOCCOCCOCCOCCOCCOCCOCCOCCOCCOCCN*]

In some embodiments, La comprises one or more amino acids selected from glycine, threonine, serine, glutamine, glutamic acid, alanine, valine, leucine, and proline and combinations thereof. In some embodiments, the La comprises 1 to 10 glycines and 1 to 6 serines. In some embodiments, the La comprises 4 glycines and 1 serine.

In another aspect, provided herein is a pharmaceutical composition comprising the compound of any of the embodiments described herein.

In another aspect, provided herein is a pharmaceutical dosage form comprising the compound of any of the embodiments described herein.

In another aspect, provided herein is a method of selectively targeting GLP1R on a surface of a cell with the compound of any of the embodiments described herein. In some embodiments, the cell is a mammalian cell. In some embodiments, the cell is a human cell. In some embodiments, the cell is a pancreatic cell, a brain cell, a heart cell, a vascular tissue cell, a kidney cell, an adipose tissue cell, a liver cell, or a muscle cell.

In another aspect, provided herein is a method of enhancing GLP1R activity in an individual in need thereof comprising administering to the individual an effective amount of the compound of any of the embodiments described herein, the composition described herein, or the dosage form described herein.

In another aspect, provided herein is a method of lowering blood glucose levels in an individual in need thereof comprising administering to the individual an effective amount of the compound of any of the embodiments described herein, the composition described herein, or the dosage form described herein.

In another aspect, provided herein is a method of lowering body weight in an individual in need thereof comprising administering to the individual an effective amount of the compound of any of the embodiments described herein, the composition described herein, or the dosage form described herein.

In another aspect, provided herein is a method of treating a GLP1R-associated condition in an individual in need thereof comprising administering to the individual an effective amount of the compound of any of the embodiments described herein, the composition described herein, or the dosage form described herein.

In some embodiments, the GLP1R-associated condition is type II diabetes, obesity, liver disease, coronary artery disease, or kidney disease. In some embodiments, the GLP1R-associated condition is type II diabetes and/or obesity.

In some embodiments, the compound of any of the embodiments described herein, the compound described herein, the composition described herein, or the dosage form described herein is administered subcutaneously, intravenously, intradermally, intraperitoneally, or intramuscularly.

These and other aspects of the present disclosure will become apparent to those skilled in the art after a reading of the following detailed description of the disclosure, including the appended claims.

### BRIEF DESCRIPTION OF THE DRAWINGS

FIG. 1 shows a schematic representation of antibody-tethered ligand (ATL) conjugation.

FIG. 2 shows size exclusion chromatogram of an ATL conjugation mixture.

FIG. 3 shows analytical size exclusion chromatogram of a purified GLP1R ATL.

FIG. 4 shows hydrophobic interaction chromatogram of anti-GLP1R antibody and its M6324 conjugates.

FIG. 5 shows liquid chromatography electrospray ionization mass spectrum of an IdeS digested GLP1R ATL sample. The calculated average drug-to-antibody ratio (DAR) value was 1.9.

FIG. 6 shows a schematic representation of the metabolic study in hepatocytes.

FIG. 7 is a graph of c-M3190/c-M6009 over time showing conversion of c-M3190 ATL to c-M6009 in a GLP1R humanized mouse.

FIG. 8 shows relative peak abundance of c-M3190 and c-M6009, demonstrating the conversion of c-M3190 ATL to c-M6009 in a GLP1R humanized mouse over 7 days.

FIG. 9 depicts bioconjugation process for preparing antibody-tethered ligand (ATL) REGN7990-M6324 using M6324. Figure discloses SEQ ID NOS 68, 68, 69, 69 and 218, respectively, in order of appearance.

FIG. 10 shows analytical size exclusion chromatogram (SEC) for ATL REGN7990-M6324.

FIG. 11 shows bioconjugation process for preparing antibody-tethered ligand (ATL) REGN15869-M6447. Figure discloses SEQ ID NOS 68, 68, 350, 350, and 336, respectively, in order of appearance.

FIG. 12 shows analytical size exclusion chromatogram (SEC) for REGN15869-M6447.

FIG. 13 shows the in vivo efficacy study of M6324-GLP1R ATL on percent body weight changes in obese GLP1R humanized mice.

FIG. 14 shows the in vivo efficacy study of M6324-GLP1R ATL on blood glucose levels in obese GLP1R humanized mice.

FIG. 15 demonstrates the effects of a group of GLP1R ATLs on percent body weight changes in obese GLP1R humanized mice.

FIG. 16 shows effects of a selected group of GLP1R ATLs (REGN7990-M3190, REGN7990-M6324, REGN7990-M6447, REGN15869-M6324, and REGN15869-M6447) on percent body weight changes in obese and diabetic NHPs.

FIG. 17 shows effects of a selected group of GLP1R ATLs (REGN7990-M3190, REGN7990-M6324, REGN7990-M6447, REGN15869-M6324, and REGN15869-M6447) on fasting glucose levels in obese and diabetic NHPs.

### DETAILED DESCRIPTION

The present disclosure provides, in some aspects, antibody-drug conjugates that specifically bind the glucagon-like peptide 1 receptor (GLP1R) protein. As described in the Background section above, GLP1R and its ligand GLP1 are highly validated targets for obesity and type 2 diabetes. However, no direct agonist antibodies have been identified for type 2 diabetes treatment. Single peptides with agonist activities on GLP1R are effective therapeutic agents for glucose control and body weight loss, but in-line peptide-antibody fusions are susceptible to proteolysis. In certain embodiments of the present disclosure, antibody-drug conjugates were generated that combine an antibody, or antigen-binding fragment thereof, specifically targeting the extracellular domain of GLP1R, with a GLP1 peptidomimetic functionally activating GLP1R. In certain embodiments, antibody-drug conjugates of the present disclosure have a longer drug duration with comparable or better weight and glucose reducing efficacy and minimized off-target side effects.

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

### Definitions

Unless defined otherwise, all technical and scientific terms used herein have the same meaning as commonly understood by one of ordinary skill in the art to which this disclosure belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder, or condition developing in a subject that may be afflicted with or predisposed to the state, disorder, or condition but does not yet experience or display clinical or subclinical symptoms of the state, disorder, or condition; or (2) inhibiting the state, disorder, or condition, i.e., arresting, reducing or delaying the development of the disease or a relapse thereof or at least one clinical or sub-clinical symptom thereof, or (3) relieving the disease, i.e., causing regression of the state, disorder, or condition or at least one of its clinical or sub-clinical symptoms. The benefit to a subject to be treated is either statistically significant or at least perceptible to the patient or to the physician. In some embodiments, treatment comprises methods wherein cells are ablated in such manner where disease is indirectly impacted. In certain embodiments, treatment comprises depleting immune cells as a hematopoietic conditioning regimen prior to therapy.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable salt”, as used in connection with compositions of the disclosure, refers to any salt suitable for administration to a patient. Suitable salts include, but are not limited to, those disclosed in. Berge et al., “Pharmaceutical Salts”, J. Pharm. Sci., 1977, 66:1, incorporated herein by reference. Examples of salts include, but are not limited to, acid derived, base derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, para-toluene sulfonic acid, salicylic acid, and the like. In some examples, a payload described herein comprises a tertiary amine, where the nitrogen atom in the tertiary amine is the atom through which the payload is bonded to a linker or a linker-spacer. In such instances, bonding to the tertiary amine of the payload yields a quaternary amine in the linker-payload molecule. The positive charge on the quaternary amine can be balanced by a counter ion (e.g., chloro, bromo, iodo, or any other suitably charged moiety such as those described herein).

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “adduct” of the present disclosure encompasses any moiety comprising the product of an addition reaction, independent of the synthetic steps taken to produce the moiety.

The term “covalent attachment” means formation of a covalent bond, i.e., a chemical bond that involves sharing of one or more electron pairs between two atoms. Covalent bonding may include different interactions, including but not limited to a-bonding, 7r-bonding, metal-to-metal bonding, agostic interactions, bent bonds, and three-center two-electron bonds. When a first group is said to be “capable of covalently attaching” to a second group, this means that the first group is capable of forming a covalent bond with the second group, directly or indirectly, e.g., through the use of a catalyst or under specific reaction conditions. Non-limiting examples of groups capable of covalently attaching to each other may include, e.g., an amine and a carboxylic acid (forming an amide bond), a maleimide and a thiol (forming a thio-maleimide), and an azide and an alkyne (forming a triazole via a 1,3-cycloaddition reaction).

The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

Unless otherwise stated, cyclic adducts, e.g., products of a cycloaddition reaction, e.g., an azide-acetylene cycloaddition reaction, depicted herein include all regioisomers, i.e., structural isomers that differ only in the position of a functional group or a substituent. By way of an example, the following structures represent triazole regioisomers, which differ only in the position of the substituent on the triazole ring:

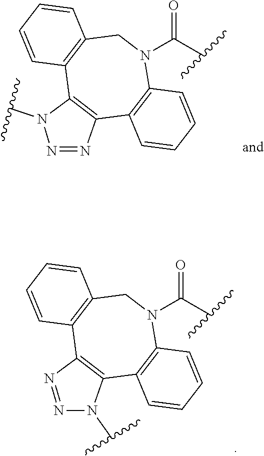 [SMILES: *C(=O)N1Cc2ccccc2-c2c(nnn2*)-c2ccccc21 ; *C(=O)N1Cc2ccccc2-c2nnn(*)c2-c2ccccc21]

Triazole regioisomers may also be represented by the following structure:

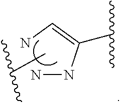

Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this disclosure.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the disclosure and salts thereof are also within the scope of the disclosure. The compounds of the disclosure may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” is meant that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

Certain groups, moieties, substituents, and atoms are depicted with a wavy line. The wavy line can intersect or cap a bond or bonds. The wavy line indicates the atom through which the groups, moieties, substituents, or atoms are bonded. For example, a phenyl group that is substituted with a propyl group depicted as:

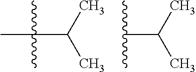

has the following structure:

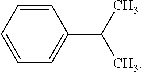

The term “GLP1R” refers to the glucagon-like peptide 1 receptor and includes recombinant GLP1R protein or a fragment thereof. GLP1R has a sequence of 463 residues. Donnelly, Br J Pharmacol, 166(1):27-41 (2011). Glucagon-like peptide 1 (GLP1) is a 31-amino acid peptide hormone released from intestinal L cells following nutrient consumption. The binding of GLP1 to GLP1R potentiates glucose-induced secretion of insulin from pancreatic beta cells, increases insulin expression, inhibits beta-cell apoptosis, promotes beta-cell neogenesis, reduces glucagon secretion, delays gastric emptying, promotes satiety, and increases peripheral glucose disposal.

The phrase “an antibody that binds GLP1R” or an “anti-GLP1R antibody” includes antibodies and antigen-binding fragments thereof that specifically recognize GLP1R.

An “agonist antibody,” as used herein (or an “antibody that increases or enhances GLP1R activity”), is intended to refer to an antibody whose binding to GLP1R results in activation of at least one biological activity of GLP1R. For example, an agonist antibody of GLP1R may elicit stimulation of the adenylate cyclase pathway resulting in increased synthesis of cyclic AMP and release of insulin if the cell is a mammalian pancreatic beta cell. An agonist antibody of GLP1R may also reduce glucose levels upon administration to a subject in need thereof.

All amino acid abbreviations used in this disclosure are those accepted by the United States Patent and Trademark Office as set forth in 37 C.F.R. § 1.822 (B)(J).

The term “protein” means any amino acid polymer having more than about 20 amino acids covalently linked via amide bonds. As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins can be produced using recombinant cell-based production systems, such as the insect bacculovirus system, yeast systems (e.g, Pichia sp.), mammalian systems (e.g., CHO cells and CHO derivatives like CHO-K1 cells).

All references to proteins, polypeptides and protein fragments herein are intended to refer to the human version of the respective protein, polypeptide or protein fragment unless explicitly specified as being from a non-human species. Thus, the expression “GLP1R” means human GLP1R unless specified as being from a non-human species, e.g., “mouse GLP1R,” “monkey GLP1R,” etc.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The term “glutaminyl-modified antibody” refers to an antibody with at least one covalent linkage from a glutamine side chain to a primary amine compound of the present disclosure. In particular embodiments, the primary amine compound is linked through an amide linkage on the glutamine side chain. In certain embodiments, the glutamine is an endogenous glutamine. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags, or TGase recognition tag).

The term “TGase recognition tag” refers to a sequence of amino acids comprising an acceptor glutamine residue and that when incorporated into (e.g. appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the TGase recognition tag. In some embodiments, the TGase recognition tag comprises at least one Gln. In some embodiments, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Gin, He, Met, Pro, Thr, Lys, or Trp or nonconventional amino acid). In some embodiments, the acyl donor glutamine-containing tag comprises an amino acid sequence selected from the group consisting of LLQGG (SEQ ID NO: 4), LLQG (SEQ ID NO: 5), LSLSQG (SEQ ID NO: 6), GGGLLQGG (SEQ ID NO: 7), GLLQG (SEQ ID NO: 8), LLQ, GSPLAQSHGG (SEQ ID NO: 9), GLLQGGG (SEQ ID NO: 10), GLLQGG (SEQ ID NO: 11), GLLQ (SEQ ID NO: 12), LLQLLQGA (SEQ ID NO: 13), LLQGA (SEQ ID NO: 14), LLQYQGA (SEQ ID NO: 15), LLQGSG (SEQ ID NO: 16), LLQYQG (SEQ ID NO: 17), LLQLLQG (SEQ ID NO: 18), SLLQG (SEQ ID NO: 19), LLQLQ (SEQ ID NO: 20), LLQLLQ (SEQ ID NO: 21), and LLQGR (SEQ ID NO: 22). See for example, WO2012059882, the entire contents of which are incorporated herein.

The term “antibody,” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2, and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding portion thereof) can be identical to the human germline sequences, or can be naturally or artificially modified. An amino acid consensus sequence can be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain can be of any size or amino acid composition and will generally comprise at least one CDR which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers.

Alternatively, the antigen-binding fragment of an antibody can contain a monomeric VH or VL domain.

In certain embodiments, an antigen-binding fragment of an antibody can contain at least one variable domain covalently linked to at least one constant domain. Non-limiting, exemplary configurations of variable and constant domains that can be found within an antigen-binding fragment of an antibody of the present description include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed herein, the variable and constant domains can be either directly linked to one another or can be linked by a full or partial hinge or linker region. A hinge region can consist of at least 2 (e.g., 5, 10, 15, 20, 40, 60, or more) amino acids which result in a flexible or semi-flexible linkage between adjacent variable and/or constant domains in a single polypeptide molecule.

Moreover, an antigen-binding fragment of an antibody of the present description can comprise a homo-dimer or hetero-dimer (or other multimer) of any of the variable and constant domain configurations listed herein in non-covalent association with one another and/or with one or more monomeric VH or VL domain (e.g., by disulfide bond(s)).

As with full antibody molecules, antigen-binding fragments can be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, can be adapted for use in the context of an antigen-binding fragment of an antibody of the present description using routine techniques available in the art.

In certain embodiments, the antibodies of the description, e.g., anti-GLP1R antibodies, are human antibodies. The term “human antibody,” as used herein, is intended to include antibodies having variable and constant regions derived from human germline immunoglobulin sequences. The human antibodies of the description can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies can, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (See, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30: 105) to levels typically observed using a human IgG1 hinge. The instant description encompasses antibodies having one or more mutations in the hinge, CH2 or CH3 region which can be desirable, for example, in production, to improve the yield of the desired antibody form.

The antibodies of the description can be isolated or purified antibodies. An “isolated antibody” or “purified antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present description. For example, an antibody that has been purified from at least one component of a reaction or reaction sequence, is a “purified antibody” or results from purifying the antibody. An isolated antibody also includes an antibody in situ within a recombinant cell. Isolated antibodies are antibodies that have been subjected to at least one purification or isolation step. According to certain embodiments, an isolated antibody or purified antibody can be substantially free of other cellular material and/or chemicals.

The antibodies disclosed herein can comprise one o more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present description includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with given heavy and light chain variable region sequences, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2, or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies of the present description can contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, improved drug-to-antibody ratio (DAR) for antibody-drug conjugates, etc. Antibodies and antigen-binding fragments obtained in this general manner are encompassed within the present description.

The term “aglycosylated antibody” refers to an antibody that does not comprise a glycosylation sequence that might interfere with a transglutamination reaction, for instance an antibody that does not have saccharide group at N297 on one or more heavy chains. In particular embodiments, an antibody heavy chain has an N297 mutation. In other words, the antibody is mutated to no longer have an asparagine residue at position 297 according to the EU numbering system as disclosed by Kabat et al. In particular embodiments, an antibody heavy chain has an N297Q or an N297D mutation. Such an antibody can be prepared by site-directed mutagenesis to remove or disable a glycosylation sequence or by site-directed mutagenesis to insert a glutamine residue at site apart from any interfering glycosylation site or any other interfering structure. Such an antibody also can be isolated from natural or artificial sources. Aglycosylated antibodies also include antibodies comprising a T299 or S298P or other mutations, or combinations of mutations that result in a lack of glycosylation.

The term “deglycosylated antibody” refers to an antibody in which a saccharide group at is removed to facilitate transglutaminase-mediated conjugation. Saccharides include, but are not limited to, N-linked oligosaccharides. In some embodiments, deglycosylation is performed at residue N297. In some embodiments, removal of saccharide groups is accomplished enzymatically, included but not limited to via PNGase.

The term “epitope” refers to an antigenic determinant that interacts with a specific antigen binding site in the variable region of an antibody molecule known as a paratope. A single antigen can have more than one epitope. Thus, different antibodies can bind to different areas on an antigen and can have different biological effects. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope can include moieties of saccharides, phosphoryl groups, or sulfonyl groups on the antigen.

The terms “conjugated protein” or “conjugated antibody” as used herein refers to a protein or an antibody covalently linked to one or more chemical moieties. The chemical moiety can include an amine compound of the present disclosure. Linkers (L) and payloads (P) suitable for use with the present disclosure are described in detail herein. In particular embodiments, a conjugated antibody comprising a therapeutic moiety is an antibody-drug conjugate (ADC), or antibody-tethered ligand (ATL), or an antibody-tethered drug conjugate (ATDC), also referred to as an antibody-payload conjugate, or an antibody-linker-payload conjugate.

The term “Drug-to-Antibody Ratio” or (DAR) is the average number of therapeutic moieties, e.g., drugs, conjugated to a binding agent of the present disclosure.

The term “Linker Antibody Ratio” or (LAR), also denoted as the lower case, in some embodiments, is the average number of reactive primary amine compounds conjugated to a binding agent of the present disclosure. Such binding agents, e.g., antibodies, can be conjugated with primary amine compounds comprising, e.g., a suitable azide or alkyne. The resulting binding agent, which is functionalized with an azide or an alkyne can subsequently react with a therapeutic moiety comprising the corresponding azide or alkyne via the 1,3-cycloaddition reaction.

The phrase “pharmaceutically acceptable amount” refers to an amount effective or sufficient in treating, reducing, alleviating, or modulating the effects or symptoms of at least one health problem in a subject in need thereof. For example, a pharmaceutically acceptable amount of an antibody or antibody-drug conjugate is an amount effective for modulating a biological target using the antibody or antibody-drug-conjugates provided herein. Suitable pharmaceutically acceptable amounts include, but are not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% of an antibody or antibody-drug-conjugate provided herein.

The phrase “reaction pH” refers to the pH of a reaction after all reaction components or reactants have been added.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98%, or 99% of the nucleotide bases, as measured by any well-known algorithm of sequence identity, such as FASTA, BLAST or Gap, as discussed below. A nucleic acid molecule having substantial identity to a reference nucleic acid molecule can, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity can be adjusted upwards to correct for the conservative nature of the substitution. Means for making this adjustment are well-known to those of skill in the art. See, e.g., Pearson (1994)Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another particular algorithm when comparing a sequence of the description to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

### Protein-Drug Conjugate Compounds

According to the foregoing objective and others, the present disclosure provides protein-drug conjugate compounds, e.g., antibody-drug conjugate compounds, and precursors and intermediates thereof, pharmaceutical compositions, and methods for treating certain diseases in a subject in need of such treatment. According to the disclosure, the protein-drug conjugate compounds provided herein comprise a binding agent conjugated with a therapeutic moiety, e.g., GLP1 peptidomimetics, as described herein.

In one aspect, the present disclosure provides compounds comprising a binding agent according to the present disclosure, (e.g., an antibody or a fragment thereof), conjugated to one or more GLP1 peptidomimetics via non-cleavable linker. Illustrative non-limiting examples include Formula (A) and Formula (B) described herein. In specific embodiments of a protein-drug conjugate according to the disclosure, wherein the binding agent is an antibody, (e.g., a monoclonal antibody), the term “antibody drug conjugate” or ADC is optionally used.

In various embodiments, antibody drug conjugate or ADC of the disclosure is an antibody-tethered drug conjugate or ATDC. An ATDC is an antibody-drug conjugate wherein the drug is tethered to the antibody by a non-cleavable linker. In some embodiments, the non-cleavable linker in an ATDC of the present disclosure is stable after the ATDC is administered into the body, e.g., a human body. For example, the non-cleavable linker can be stable in plasma, e.g., in human plasma, stable upon binding cell surface, or stable upon antibody binding its target antigen and/or GLP1 peptidomimetic binding GLP1R. In some embodiments, the non-cleavable linker is more stable in vivo than either the payload or the antibody under the same physiological conditions. In some embodiments, an ATDC of the present disclosure may be degraded in the lysosome to release the payload, the linker-payload, and/or its ATDC metabolites/catabolites, which in certain embodiments are effective for GLP1R activation either locally or systematically.

In some embodiments, the ATDC is stable in plasma and degrades in the lysosome. In some embodiments, the ATDC is stable in plasma and does not degrade in the lysosome.

In one aspect, provided herein is a compound having a structure of Formula (A):

BA-(L-P)ₘ (A),
wherein:
BA is an antibody or an antigen-binding fragment thereof,
L is a non-cleavable linker;
P is a payload having the structure selected from the group consisting of:

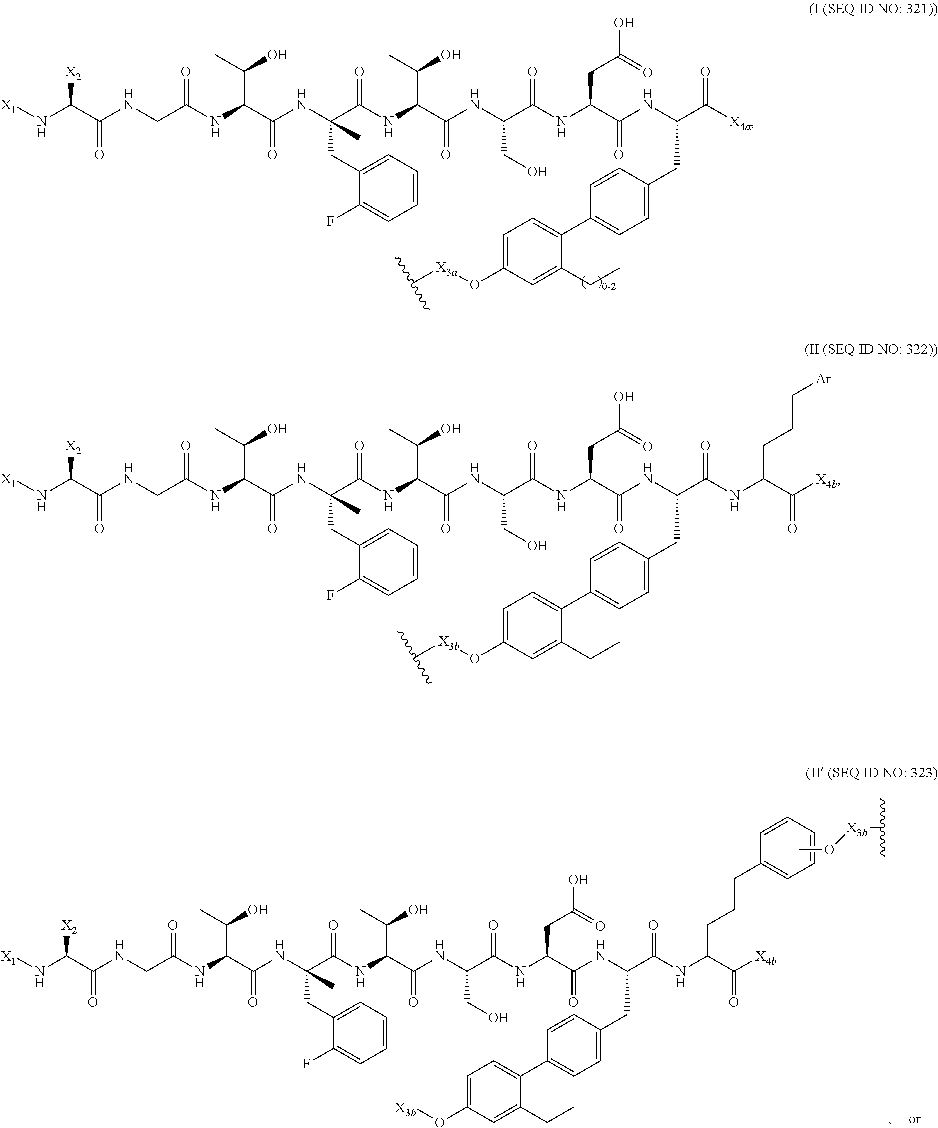

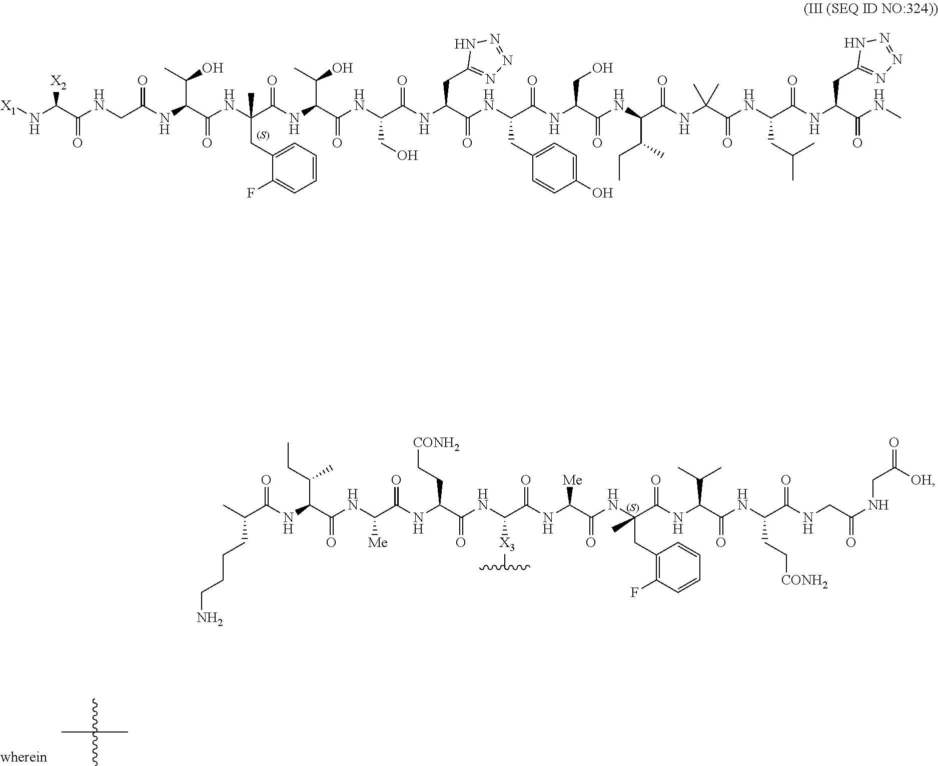

is the point of attachment of the payload to L;
X₁ is selected from H;

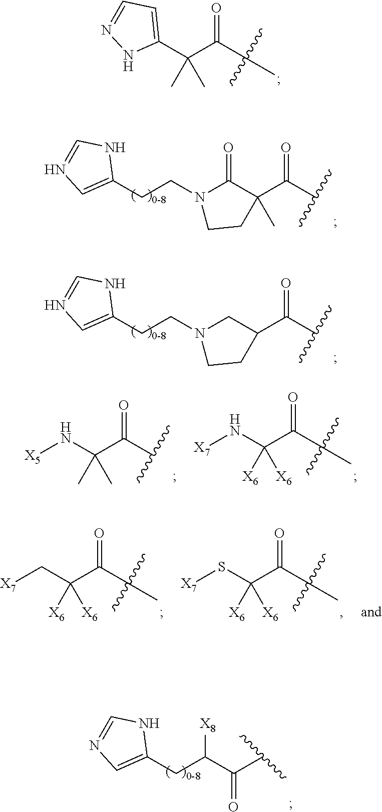

X₂ is selected from

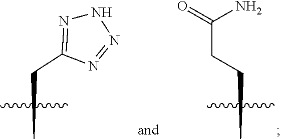

X₃ₐ is selected from bond, —CH3, —(CH2)₁₋₄(OCH₂CH2)₂₋₁₅-NH₂, —(CH2)₂₋₆-NH₂, —(CH2)₂₋₆-N₃, —(CH2)₂₋₆-Tr-(CH2)₁₋₆-NH₂, —(CH2)₂₋₆-NH—, —(CH2)₂₋₆-Tr-, and —(CH2)₂₋₆-Tr-(CH2)₁₋₆-NH—; X_{3b} is selected from bond, —CH3, —(CH2)₂₋₆-NH₂, —(CH2)₂₋₆-N₃, —(CH2)₂₋₆-Tr-(CH2)₁₋₆-NH₂, —(CH2)₂₋₆-NH—, —(CH2)₂₋₆-Tr-, and —(CH2)₂₋₆-Tr-(CH2)₁₋₆-NH—;
Tr is a triazole moiety;
X₄ₐ is —NH₂ or —OH;
X_{4b} is —OH;
X₅ is selected from —OH, —NH₂, —NH—OH, and

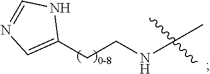

X₆ is independently at each occurrence selected from H, —OH, —CH₃, and —CH₂OH;
X₇ is selected from H,

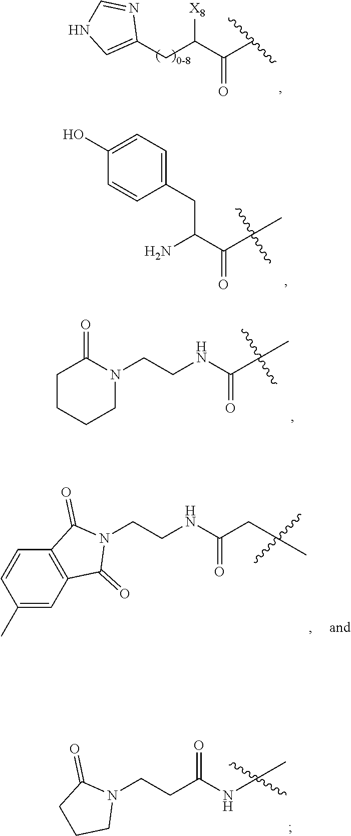

X₈ is selected from H, —OH, —NH₂, and

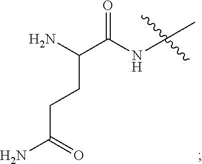 [SMILES: *NC(=O)C(N)CCC(N)=O]

Ar is selected from

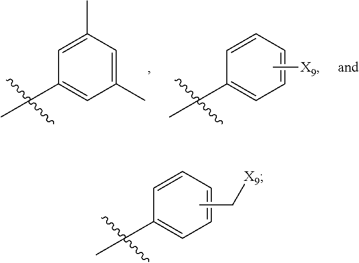

and
X₉ is selected from the group consisting of-NH₂,

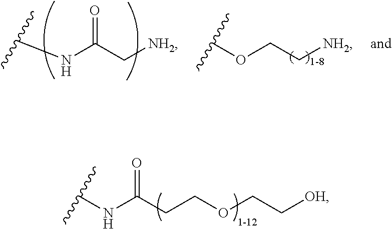

or a pharmaceutically acceptable salt thereof,
with the proviso that when with the proviso that when X_{4b} is —OH, Ar is

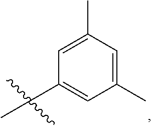 [SMILES: *c1cc(C)cc(C)c1]

### and L is

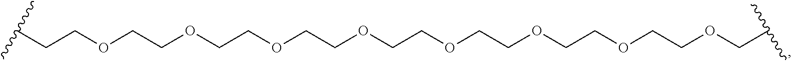 [SMILES: *CCOCCOCCOCCOCCOCCOCCOCCOC*]

### X₅ is not

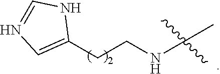

In one embodiment, m is 1. In one embodiment, m is an integer from 2 to 4. In one embodiment, m is 2.

In one aspect, the present disclosure provides a compound having a structure of Formula (B):

BA-L-P (B),
wherein:
BA is an antibody or an antigen-binding fragment thereof,
L is a non-cleavable linker;
P is a payload having the structure selected from the group consisting of:

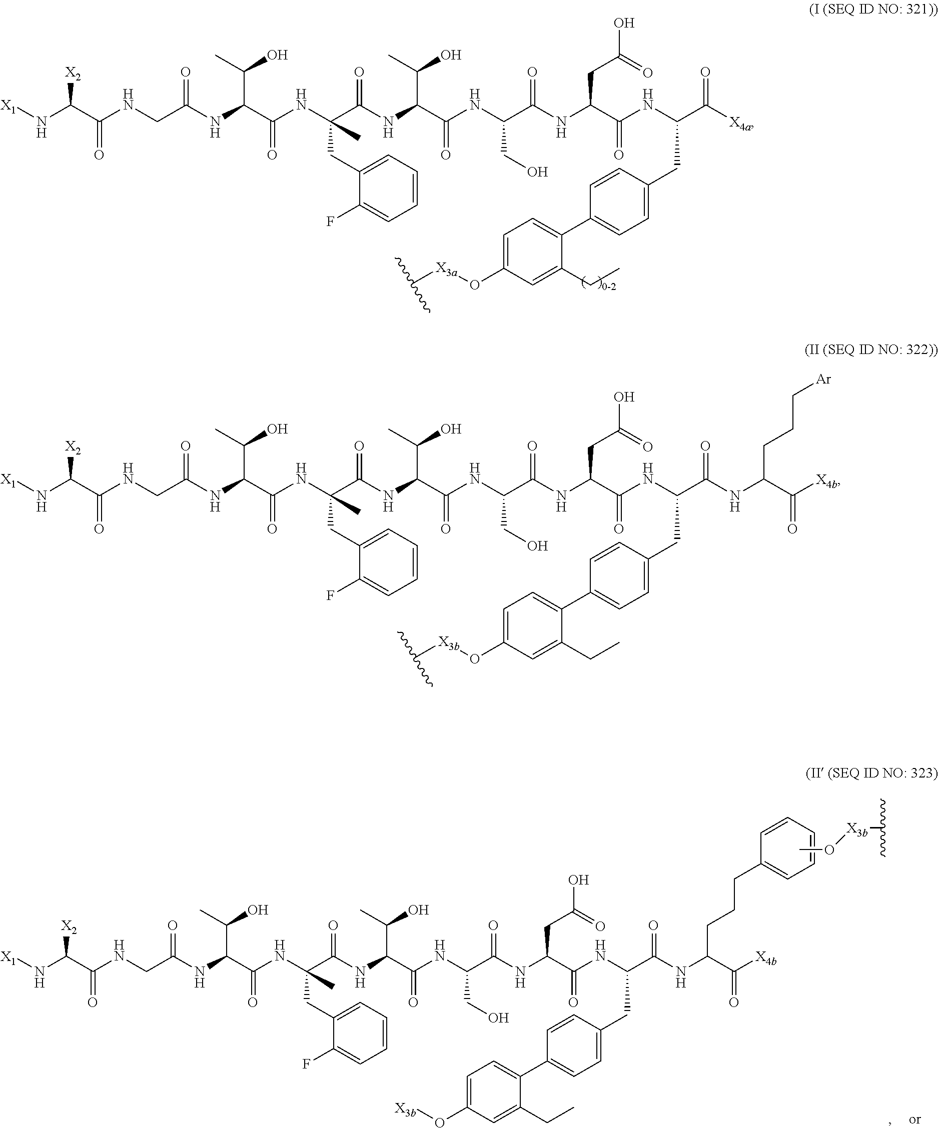

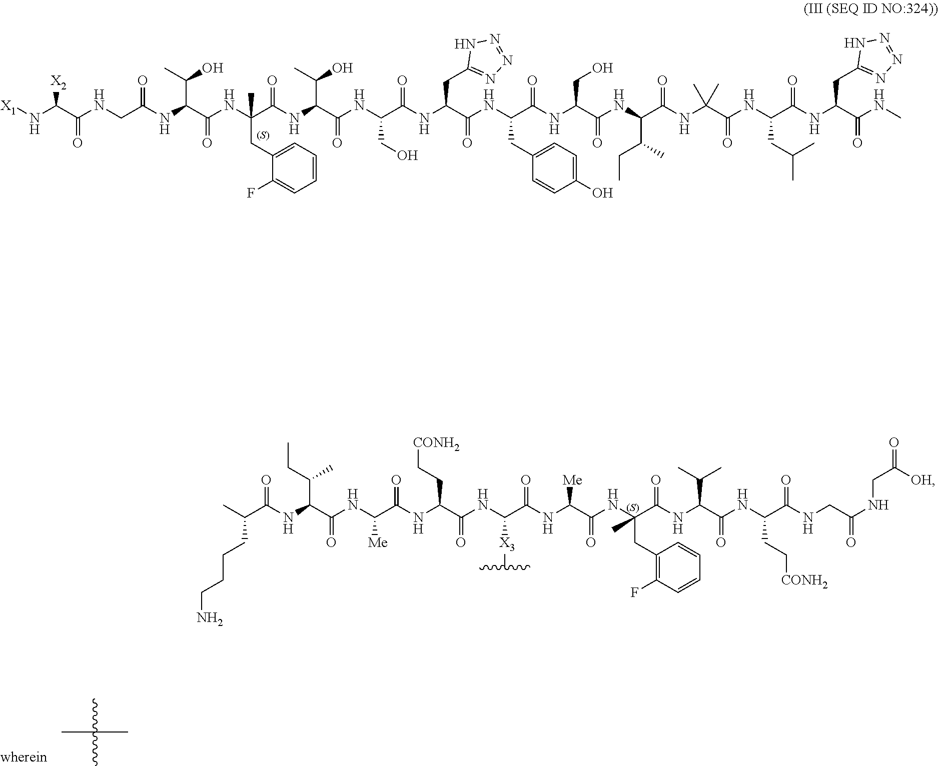

is the point of attachment of the payload to L;
X₁ is selected from H;

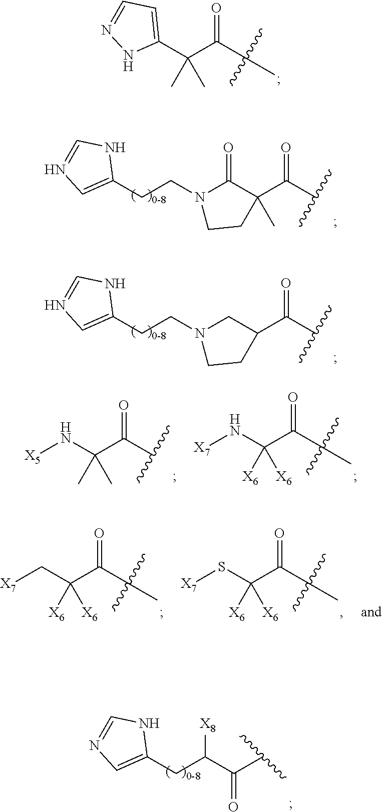

X₂ is selected from

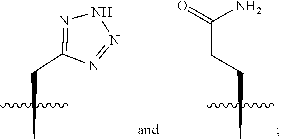

X₃ₐ is selected from bond, —CH₃, —(CH₂)₁₋₄(OCH₂CH₂)₂₋₁₅—NH₂, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—; X_{3b} is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
Tr is a triazole moiety;
X₄ₐ is —NH₂ or —OH;
X_{4b} is —OH;
X₅ is selected from —OH, —NH₂, —NH—OH, and

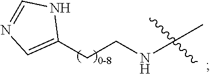

X₆ is independently at each occurrence selected from H, —OH, —CH₃, and —CH₂OH;
X₇ is selected from H,

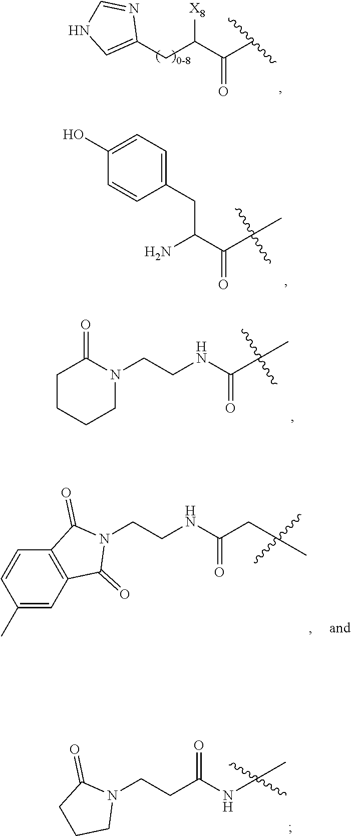

X₈ is selected from H, —OH, —NH₂, and

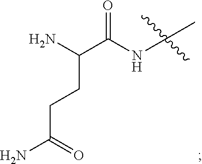 [SMILES: *NC(=O)C(N)CCC(N)=O]

Ar is selected from

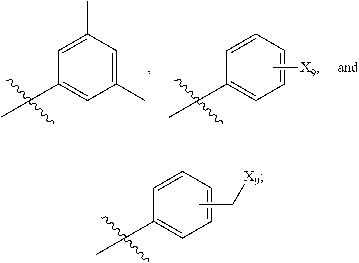

and
X₉ is selected from the group consisting of —NH₂,

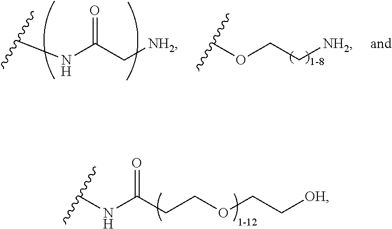

or a pharmaceutically acceptable salt thereof,
with the proviso that when with the proviso that when X_{4b} is —OH, Ar is

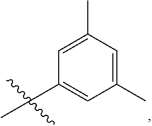 [SMILES: *c1cc(C)cc(C)c1]

### and L is

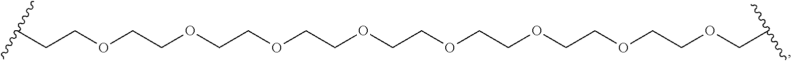 [SMILES: *CCOCCOCCOCCOCCOCCOCCOCCOC*]

### X₅ is not

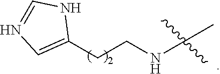

In some embodiments, P has a structure of Formula (Ia):

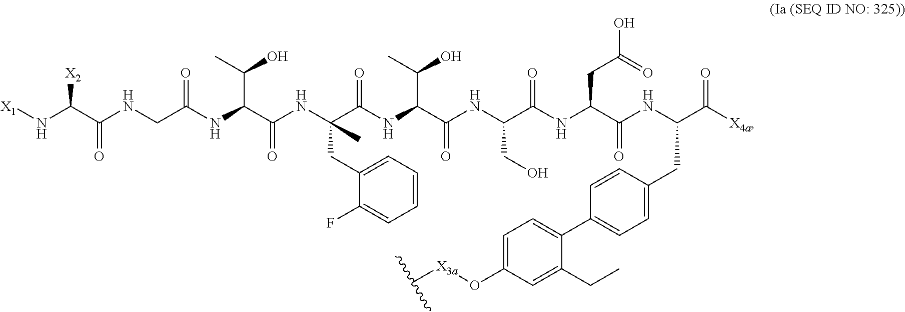

where X₃ₐ is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—.

In some embodiments, the linker L is attached to one or both heavy chains of the BA. In some embodiments, the linker L is attached to one or both heavy chain variable domains of the BA. In some embodiments, the linker L is attached to one or both light chains of the BA. In some embodiments, the linker L is attached to one or both light chain variable domains of the BA.

In some embodiments, the linker L is attached to BA via a glutamine residue. In some embodiments, the glutamine residue is introduced to the N-terminus of one or both heavy chains of the BA. In some embodiments, the glutamine residue is introduced to the N-terminus of one or both light chains of the BA. In some embodiments, the glutamine residue is naturally present in a CH2 or CH3 domain of the BA. In some embodiments, the glutamine residue is introduced to the BA by modifying one or more amino acids. In some embodiments, the glutamine residue is Q295 or N297Q.

In some embodiments, the linker L is attached to BA via a lysine residue.

In some embodiments, the linker L is attached to BA via a cysteine residue.

In some embodiments, the antibody or antigen-binding fragment thereof is aglycosylated. In some embodiments, the antibody or antigen-binding fragment thereof is deglycosylated. In some embodiments, the antigen-binding fragment is an Fab fragment.

In various embodiments of the compound described herein, the compound has a half life of longer than 7 days in plasma.

In various embodiments of the compound described herein, the compound does not bind to G protein-coupled receptors (GPCRs) other than GLP1R.

In some embodiments of the compound described herein, BA is a glucagon-like peptide-1 receptor (GLP1R)-targeting antibody or an antigen-binding fragment thereof. In some embodiments, the GLP1R-targeting antibody is a GLP1R agonist antibody. In some embodiments, the GLP1R-targeting antibody is 5A10, 9A10, AB9433-I, h38C2, PA5-111834, NLS1205, MAB2814, EPR21819, or glutazumab.

In some embodiments, BA is a glucagon-like peptide-1 receptor (GLP1R)-targeting antibody or an antigen-binding fragment thereof comprising REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280.

In some embodiments, the anti-GLP1R antibodies and antigen-binding fragments of the present invention are glutaminyl-modified. The term “glutaminyl-modified” antibody, for example, refers to an antibody (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) modified with a payload or a linker-payload (LP) as described in this disclosure, wherein the conjugation of the payload or linker-payload is through at least one covalent linkage from a glutamine side chain of said antibody or a glutamine-Tag (Q-tag)) to a primary amine compound (e.g., a Payload or Linker-Payload) of the present disclosure.

In some particular embodiments, the primary amine compound is linked through an amide linkage on the glutamine side chain. In certain embodiments, the glutamine is an endogenous glutamine. In certain embodiments, the glutamine residue is naturally present in a CH2 or CH3 domain of the BA. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags or TGase recognition tag).

In some embodiments, the conjugate compound (ADC, ATL, or ADTL) has a structure (SEQ ID NOS 326-329 disclosed below, respectively, in order of appearance)

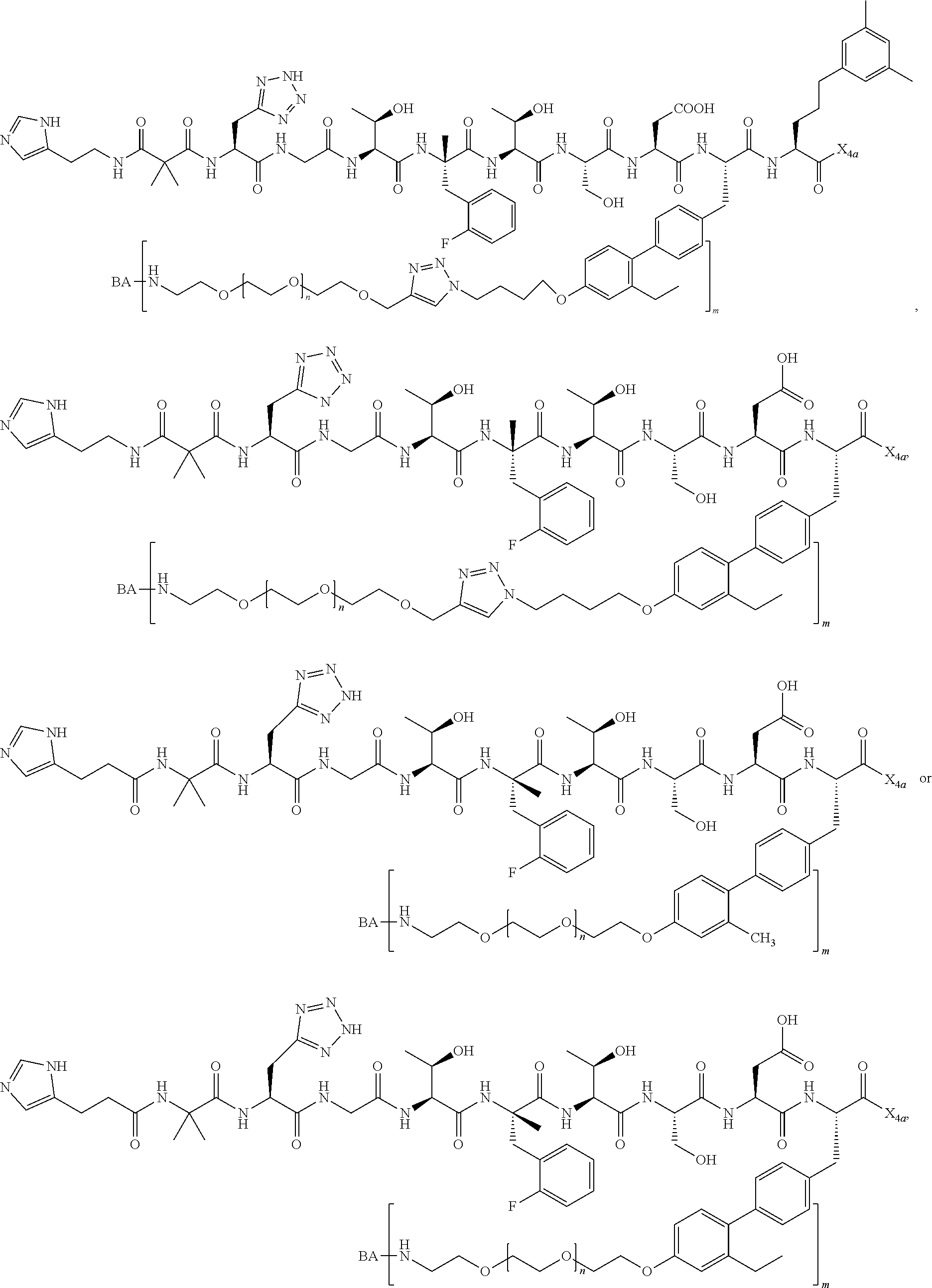

wherein X₄ₐ is —NH₂ or —OH; m ranges from about one to ten; n is an integer of one to twelve; and BA is a GLP1R antibody or an antigen binding fragment thereof. In some embodiments, m ranges from about 1 to 4, and n is six. In some other embodiments, X₄ₐ is —NH₂. In some other embodiments, X₄ₐ is-OH. Yet in some other embodiments, BA is a GLP1R antibody comprising REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; and REGN9280.

In some embodiments, the linker L is attached to BA via a lysine residue. In some other embodiments, the linker L is formed via Click Chemistry. Yet in some other embodiments, the linker L is formed via multiple steps of a process comprising Click Chemistry, transglutaminase-assisted conjugation, and chemo-selective nuleiphilic reaction through the side chain thiol group of a cysteine residue or the side chain primary amine of a lysine residue of BA.

### Linker L

In one embodiment, the linker L is a non-cleavable linker, i.e., a linker which is stable and provides a covalent connection between the antibody and the drug, e.g., between a GLP1R-targeting antibody and a GLP1 peptidomimetic payload P according to the present disclosure. In some embodiments, the non-cleavable linker L of the present disclosure is stable after the ATDC is administered into the body, e.g., a human body. For example, the linker L can be stable in plasma, e.g., in human plasma, stable upon binding cell surface, or stable upon antibody binding its target antigen and/or GLP1 peptidomimetic binding GLP1R. In some embodiments, the linker L is more stable in vivo than either the payload or the antibody under the same physiological conditions.

In one embodiment, the linker L has the structure of formula (L′):

-La-Y-Lp- (L′),
wherein La is a first linker covalently attached to the BA;
Y is a group comprising a triazole, and
Lp is absent or a second linker covalently attached to the P, wherein when Lp is absent, Y is also absent.

In another embodiment, the linker L, or the first linker La, or the second linker Lp, comprises a polyethylene glycol (PEG) segment having 1 to 36-CH₂CH₂O-(EG) units.

In one embodiment, L comprises a polyethylene glycol (PEG) segment having 1 to 36-CH₂CH₂O-(EG) units. In another embodiment, La comprises a polyethylene glycol (PEG) segment having 1 to 36-CH₂CH₂O-(EG) units. In another embodiment, Lp comprises a polyethylene glycol (PEG) segment having 1 to 36-CH₂CH₂O-(EG) units.

In one embodiment, the PEG segment comprises between 2 and 30 EG units, or between 4 and 24 EG units. In one embodiment, the PEG segment comprises 2 EG units, or 4 EG units, or 6 EG units, or 8 EG units, or 10 EG units, or 12 EG units, or 14 EG units, or 16 EG units, or 18 EG units, or 20 EG units, or 22 EG units, or 24 EG units.

In another embodiment, the PEG segment comprises 4 EG units, 5 EG units, 6 EG units, 7 EG units, 8 EG units, 9 EG units, 10 EG units, 11 EG units, or 12 EG units.

In another embodiment, the PEG segment comprises 4 EG units. In one embodiment, the PEG segment comprises 8 EG units. In one embodiment, the PEG segment comprises 12 EG units. In one embodiment, the PEG segment comprises 24 EG units.

In one embodiment, the PEG segment comprises 4 to 8 EG units. In one embodiment, the PEG segment comprises 4 EG units or 8 EG units.

In some embodiments, Lp comprises a polyethylene glycol (PEG) segment having 1 to 36-CH₂CH₂O-(EG) units. In some embodiments, the PEG segment comprises between 2 and 30 EG units. In some embodiments, the PEG segment comprises between 4 and 24 EG units. In some embodiments, the PEG segment comprises 4 EG units, 5 EG units, 6 EG units, 7 EG units, 8 EG units, 9 EG units, 10 EG units, 11 EG units, or 12 EG units. In some embodiments, the PEG segment comprises 4 EG units. In some embodiments, the PEG segment comprises 8 EG units.

In another embodiment, La comprises a PEG segment having 3 EG units.

In another embodiment, La has a structure selected from the group consisting of:

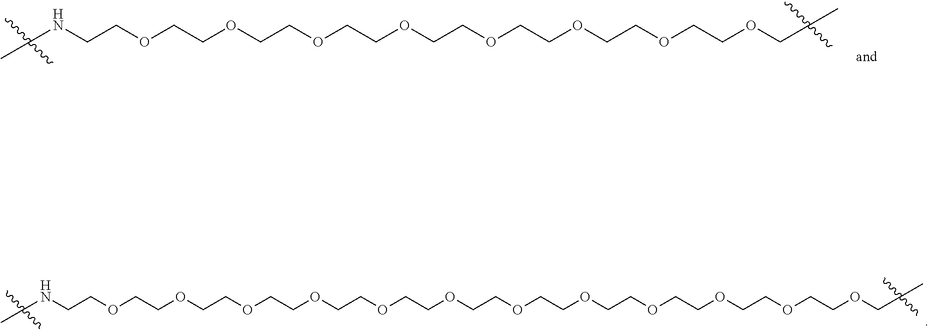 [SMILES: *COCCOCCOCCOCCOCCOCCOCCOCCN* ; *COCCOCCOCCOCCOCCOCCOCCOCCOCCOCCOCCOCCN*]

In another embodiment, the linker L or the first linker La, or the second linker Lp, comprises one or more amino acids selected from glycine, threonine, serine, glutamine, glutamic acid, alanine, valine, leucine, and proline and combinations thereof.

In one embodiment, L comprises one or more amino acids selected from glycine, threonine, serine, glutamine, glutamic acid, alanine, valine, leucine, and proline and combinations thereof. In another embodiment, La comprises one or more amino acids selected from glycine, threonine, serine, glutamine, glutamic acid, alanine, valine, leucine, and proline and combinations thereof. In another embodiment, Lp comprises one or more amino acids selected from glycine, threonine, serine, glutamine, glutamic acid, alanine, valine, leucine, and proline and combinations thereof.

In some embodiments, the Lp comprises 1 to 10 glycines. In some embodiments, the Lp comprises 1 to 6 serines. In some embodiments, the Lp comprises 1 to 10 glycines and 1 to 6 serines. In some embodiments, the Lp comprises 4 glycines and 1 serine.

In one embodiment, the linker L or the first linker La, or the second linker Lp, comprises from 1 to 10 glycines, or 1 glycine, or 2 glycines, or 3 glycines, or 4 glycines, or 5 glycines, or 6 glycines, or 7 glycines, or 8 glycines, or 9 glycines, or 10 glycines.

In one embodiment, the linker L or the first linker La, or the second linker Lp, comprises from 1 to 6 serines, or 1 serine, or 2 serines, or 3 serines, or 4 serines, or 5 serines, or 6 serines.

In one embodiment, the linker L or the first linker La, or the second linker Lp, comprises 1 to 10 glycines and 1 to 6 serines.

In one embodiment, the linker L or the first linker La, or the second linker Lp, comprises 4 glycines and 1 serine.

In one embodiment, the linker L or the first linker La, or the second linker Lp, is selected from the group consisting of Gly-Gly-Gly-Gly-Ser (G4S) (SEQ ID NO: 1), Ser-Gly-Gly-Gly-Gly (SG4) (SEQ ID NO: 2), and Gly-Gly-Gly-Gly-Ser-Gly-Gly-Gly-Gly-Ser (G4S-G4S) (SEQ ID NO: 3).

In one embodiment, the linker L or the first linker La, or the second linker Lp, comprises from 1 to 10 glutamic acids and from 1 to 10 glycines.

In some embodiments, the linker L or the first linker La, or the second linker Lp, comprises a combination of a polyethylene glycol (PEG) segment having 1 to 36-CH₂CH₂O-(EG) units and one or more amino acids selected from glycine, serine, glutamic acid, alanine, valine, and proline and combinations thereof.

In some embodiments, the Lp comprises a combination of a PEG segment having 1 to 36 EG units and one or more amino acids selected from glycine, serine, glutamic acid, alanine, valine, threonine, leucine, and proline, and combinations thereof.

In one embodiment, the linker L or the first linker La, or the second linker Lp, comprises a combination of a PEG segment having 1 to 36 EG units and 1 to 10 glycines. In one embodiment, the linker L or the first linker La, or the second linker Lp, comprises a combination of a PEG segment having 1 to 36 EG units and a group selected from Gly-Gly-Gly-Gly-Ser (G4S) (SEQ ID NO: 1), Ser-Gly-Gly-Gly-Gly (SG4) (SEQ ID NO: 2), and Gly-Gly-Gly-Gly-Ser-Gly-Gly-Gly-Gly-Ser (G4S-G4S) (SEQ ID NO: 3).

In some embodiments, the serine residue comprises a carbohydrate group, e.g., a glucose group.

In some embodiments, the serine residue comprises a glucose group.

In some embodiments, the Lp comprises a combination of a PEG segment having 1 to 36 EG units and a triazole group.

In some embodiments, the Lp comprises a combination of a PEG segment having 1 to 36 EG units; a triazole group; and —CH₂—O— group.

In some embodiments, the Lp comprises a combination of a PEG segment having 1 to 36 EG units; a triazole group; —CH₂—O— group; and —(CH₂)₁₋₂₄— group.

In some embodiments, the Lp comprises a combination of a PEG segment having 5 to 10 EG units; a triazole group; —CH₂—O— group; and —(CH₂)₁₋₅— group.

In some embodiments, the Lp is

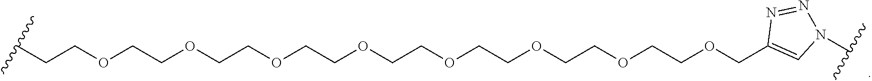

In one embodiment, the linker L or the first linker La, or the second linker Lp, has a structure selected from the group consisting of:

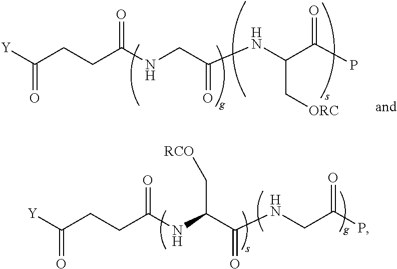

wherein Y is the group comprising a triazole, e.g., as shown above, and P is the payload, and wherein Rc is selected from hydrogen (H) and glucose, g is an integer from 1 to 10 and s is an integer from 0 to 4.

In another embodiment, L has a structure selected from the group consisting of:

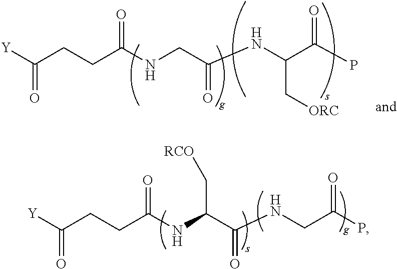

wherein Y is the group comprising a triazole and P is the payload, and wherein Rc is selected from H and glucose, g is an integer from 1 to 10 and s is an integer from 0 to 4.

In another embodiment, La has a structure selected from the group consisting of:

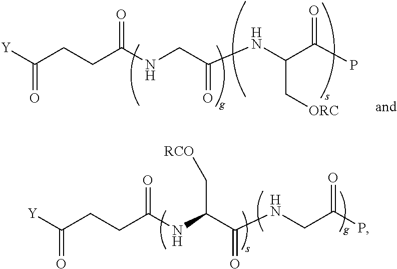

wherein Y is the group comprising a triazole and P is the payload, and wherein Rc is selected from H and glucose, g is an integer from 1 to 10 and s is an integer from 0 to 4.

In another embodiment, Lp has a structure selected from the group consisting of:

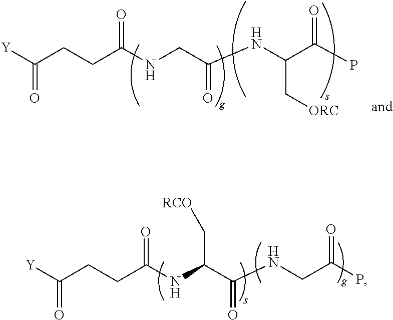

wherein Y is the group comprising a triazole and P is the payload, and wherein Rc is selected from H and glucose, g is an integer from 1 to 10 and s is an integer from 0 to 4.

In one embodiment, the linker L comprises a cyclodextrin moiety.

In one embodiment, Y is a group comprising a triazole.

In yet another embodiment, Y has a structure selected from the group consisting of:

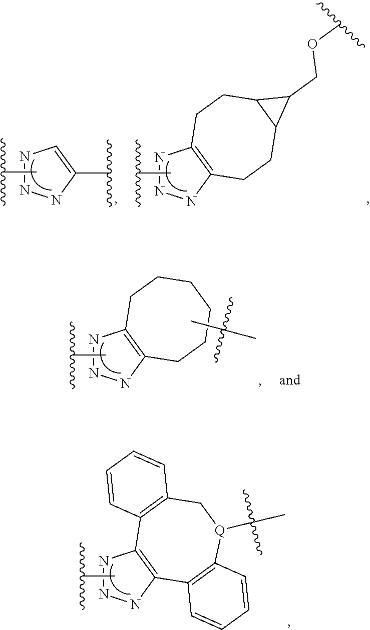

wherein Q is C or N.

In one embodiment, Y-Lp is absent.

In another embodiment, Y-Lp has a structure selected from the group consisting of:

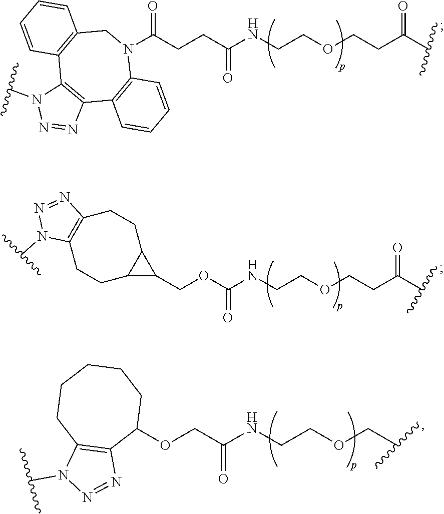

or a triazole regioisomer thereof,
wherein p is an integer from 1 to 36.

In another embodiment, Y-Lp has a structure selected from the group consisting of (SEQ ID NOS 330-335 disclosed below, respectively, in order of appearance):

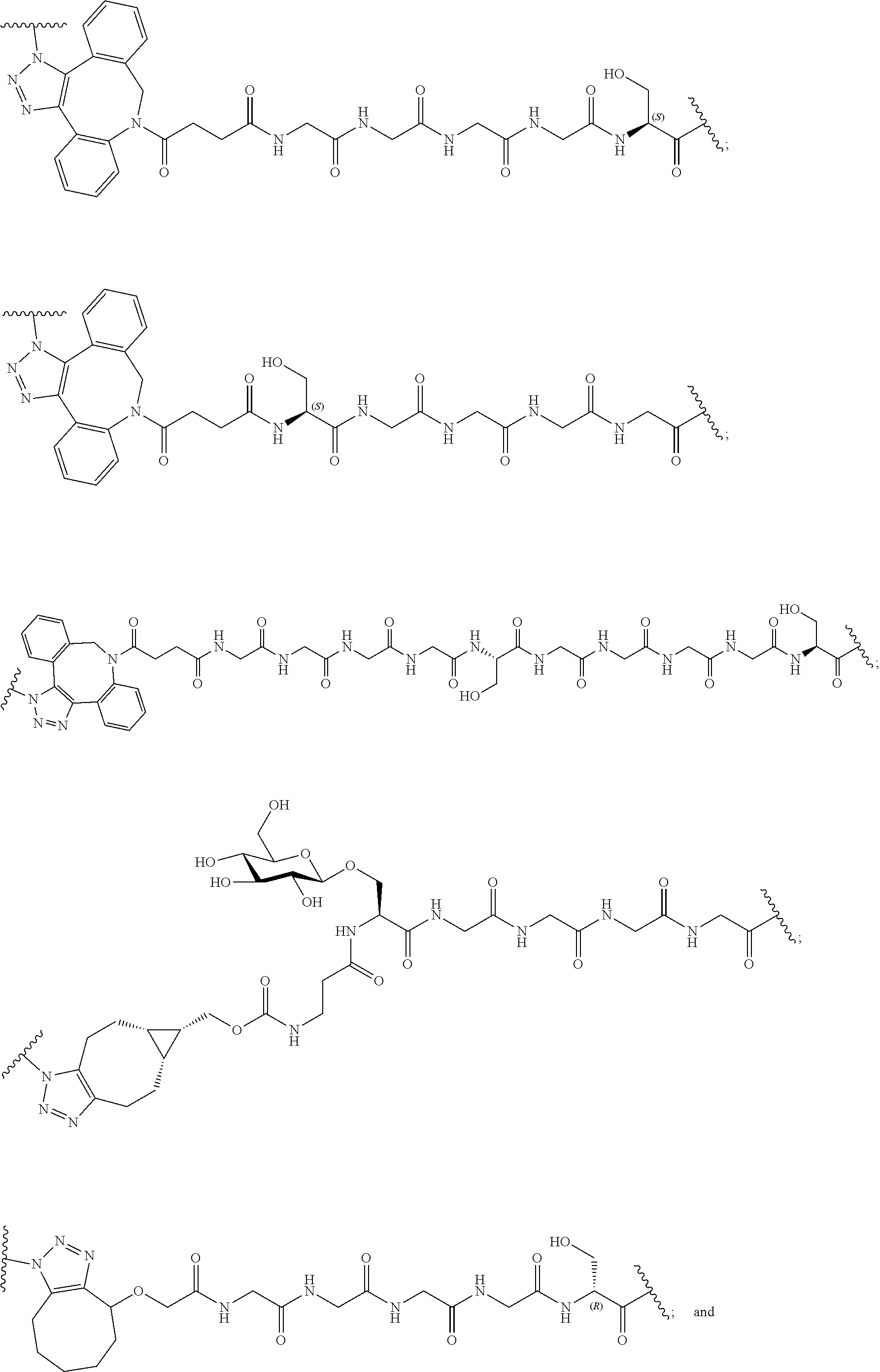

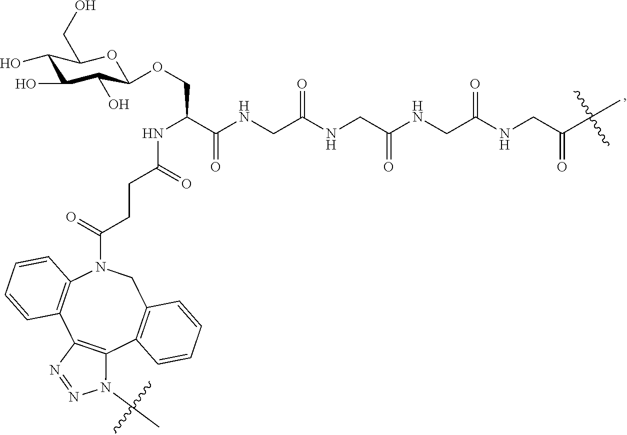

or a triazole regioisomer thereof.

### Payloads P

In one aspect, the payloads P according to the present disclosure have a structure of Formula (P-I), Formula (P-II), Formula (P-III), or Formula (P-IV):

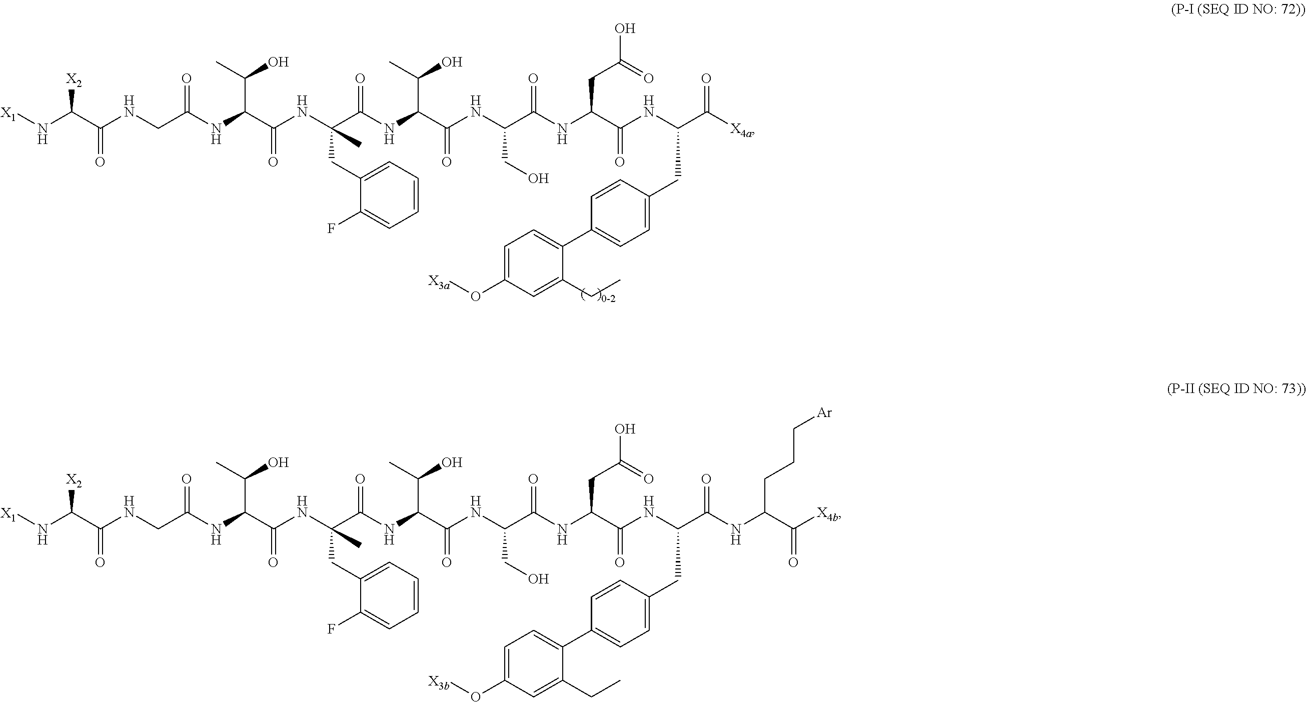

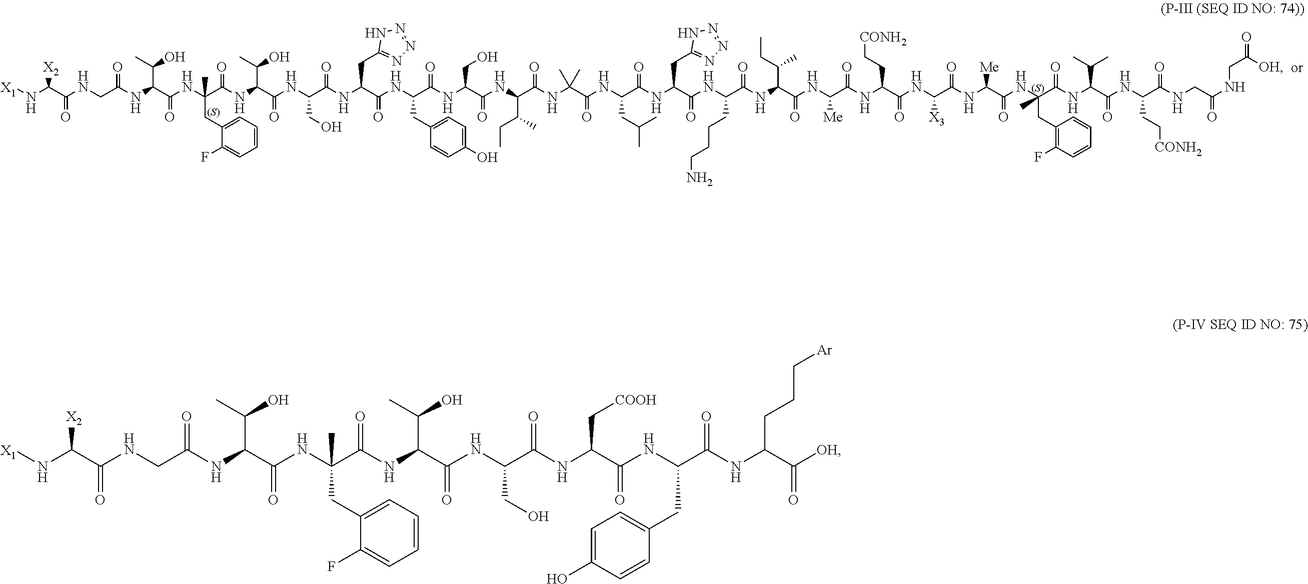

wherein:
X₁ is selected from H;

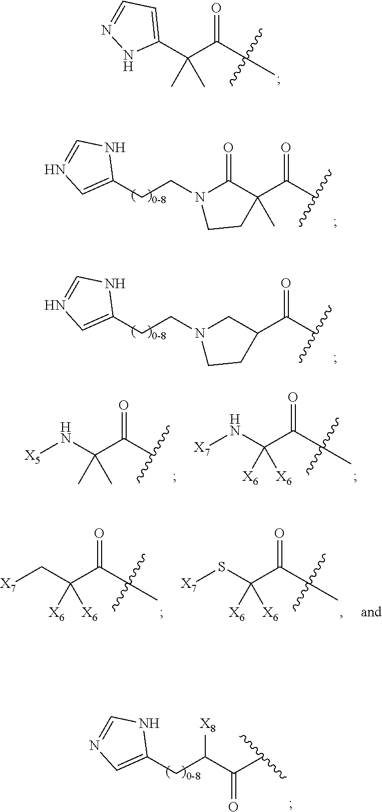

X₂ is selected from

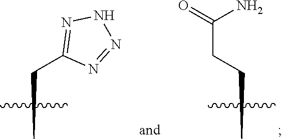

X₃ₐ is selected from —CH₃, —(CH₂)₁₋₄(OCH₂CH₂)₂₋₁₅—NH₂, —(CH₂)₂₋₆—NH₂, —(CH₂)₂₋₆—N₃, and —(CH₂)₂₋₆-Tr-(CH₂)₁₋₆—NH₂;
X_{3b} is selected from —CH₃, —(CH₂)₂₋₆—NH₂, —(CH₂)₂₋₆—N₃, and —(CH₂)₂₋₆-Tr-(CH₂)₁₋₆—NH₂;
Tr is a triazole moiety; X₄ₐ is —NH₂ or —OH;
X_{4b} is —OH;
X₅ is selected from —OH, —NH₂, —NH—OH, and

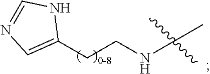

X₃ is independently at each occurrence selected from H, —OH, —CH₃, and —CH₂OH;
X₇ is selected from H,

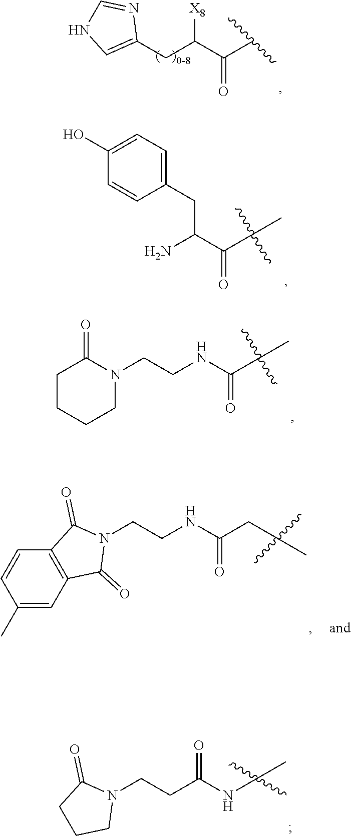

X₈ is selected from H, —OH, —NH₂, and

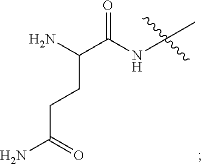 [SMILES: *NC(=O)C(N)CCC(N)=O]

Ar is selected from

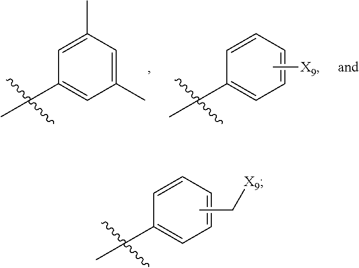

and
X₉ is selected from —NH₂,

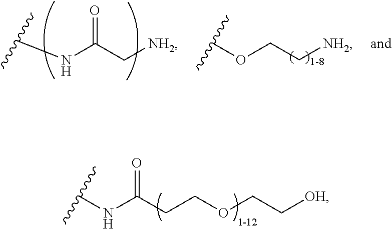

or a pharmaceutically acceptable salt thereof,
with the proviso that when X_{4b} is —OH, and Ar is

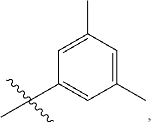 [SMILES: *c1cc(C)cc(C)c1]

### X₅ is not

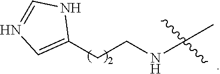

The payloads P according to the present disclosure can be prepared as follows. A peptide acid (the C-terminal end is an acid) can be prepared using a chlorotrityl chloride (CTC) resin. A peptide amide (the C-terminal end is an amide) can be synthesized using a Rink amide resin. The standard coupling cycles of peptide chain assembly are roughly the same for the synthesis of a peptide acid and a peptide amide. The detailed synthetic process for the preparation of a peptide amide and other compounds can be found in U.S. Patent Application Publication No. 2022/0096648 and U.S. Patent Application Publication No. 2023/0330254, both of which are incorporated herein by reference in their entirety.

In some embodiments, the compound of Formula (P-I) has a structure of Formula (P-Ia):

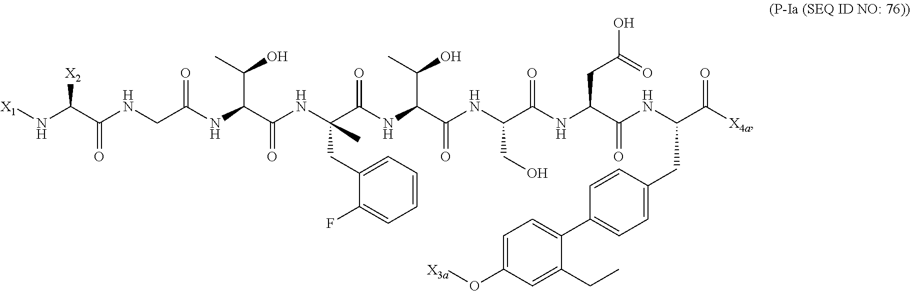

wherein X₃ₐ is selected from —CH₃, —(CH₂)₂₋₆—NH₂, —(CH₂)₂₋₆—N₃, and —(CH₂)₂₋₆-Tr-(CH₂)₁₋₆—NH₂.

In some embodiments, the compound of Formula (P-I) has a structure of Formula (P-Ib):

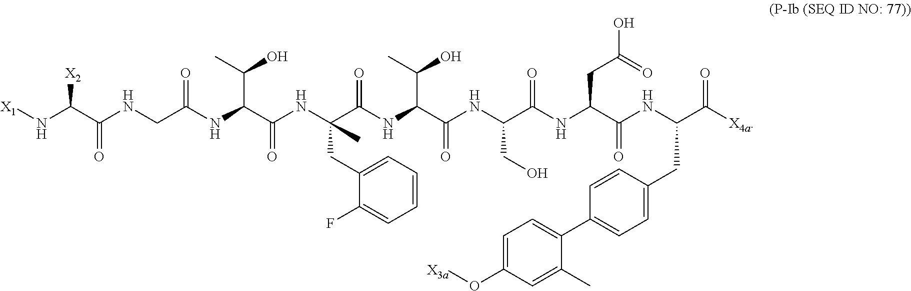

In one embodiment, X₁ is

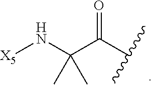

In another embodiment, X₁ is

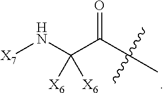

In another embodiment, X₂ is

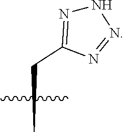

In yet another embodiment, X₃ₐ is —(CH₂)₂₋₆—N₃.

In a further embodiment, X₃ₐ is —(CH₂)₁₋₄(OCH₂CH₂)₂₋₁₅—NH₂.

In another embodiment, X_{3b} is —(CH₂)₂₋₆—N₃.

In yet another embodiment, X₅ is

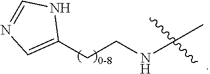

In another embodiment, X₇ is

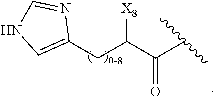

In a further another embodiment, X₄ₐ is —NH₂.

In another embodiment, X₄ₐ is-OH.

In yet another embodiment, the payloads P according to the present disclosure have a structure selected from:

| P# | Structure |
|---|---|
| P0 (SEQ ID NO: 146) | 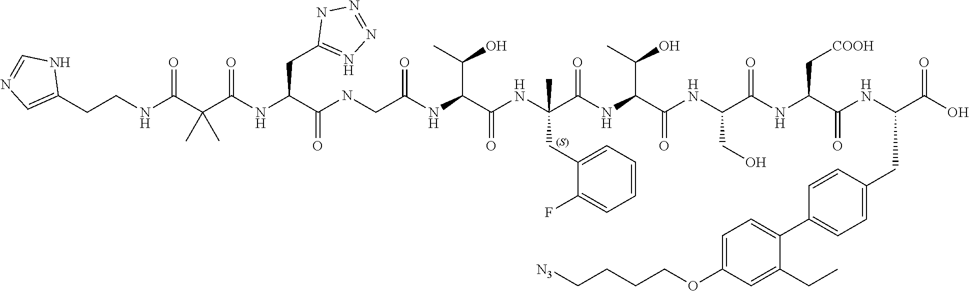 |
| P1 (SEQ ID NO: 147) | 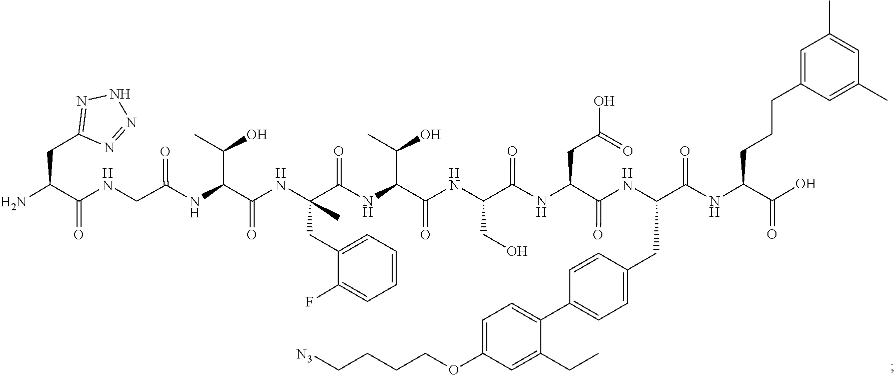 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(=O)O)cc1] |
| P2 (SEQ ID NO: 148) | 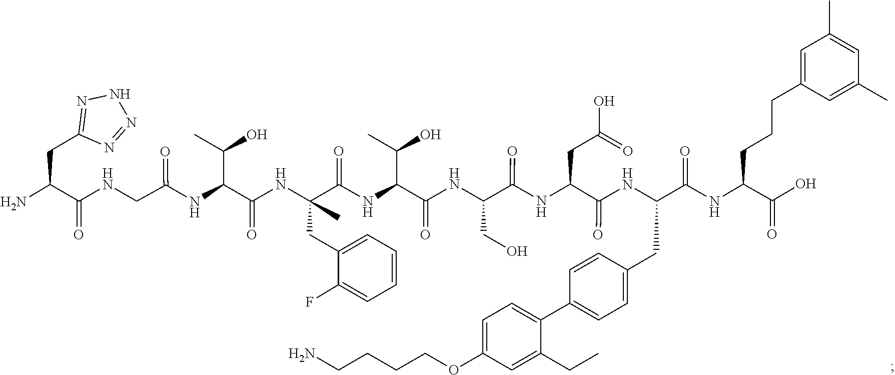 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@@H](N)Cc2nn[nH]n2)[C@@H](C)O)[C@@H](C)O)C(=O)N[C@@H](CCCc2cc(C)cc(C)c2)C(=O)O)cc1] |
| P3 (SEQ ID NO: 149) | 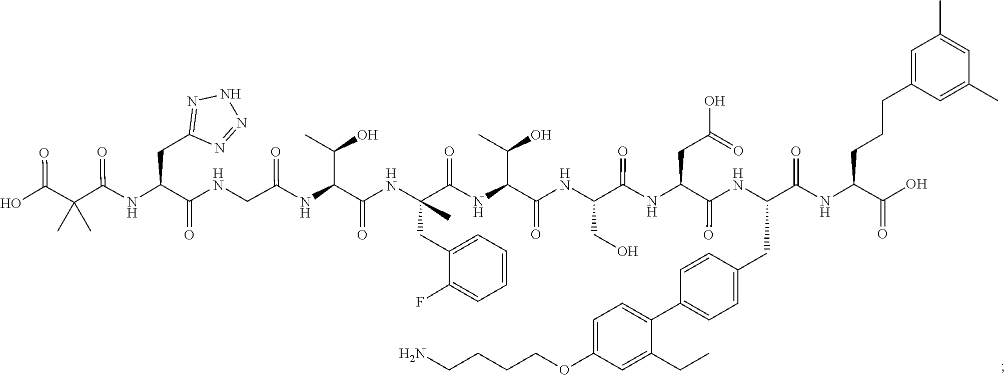 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)O)[C@@H](C)O)[C@@H](C)O)C(=O)N[C@@H](CCCc2cc(C)cc(C)c2)C(=O)O)cc1] |
| P4 (SEQ ID NO: 150) | 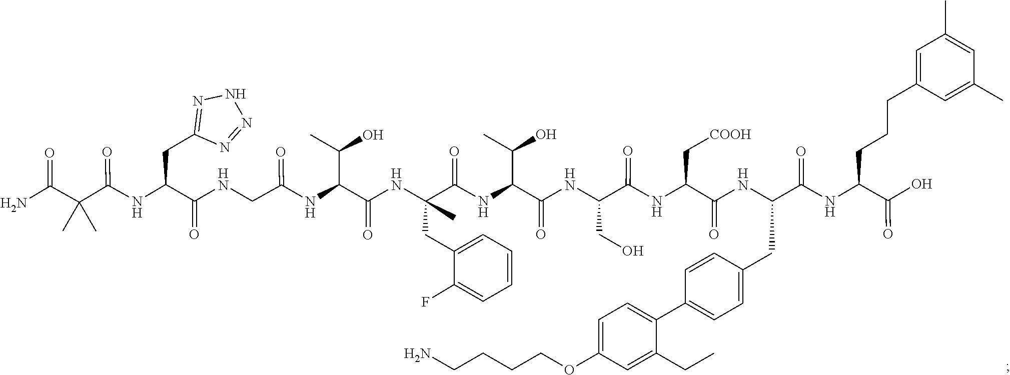 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(=O)O)cc1] |
| P5 (SEQ ID NO: 151) | 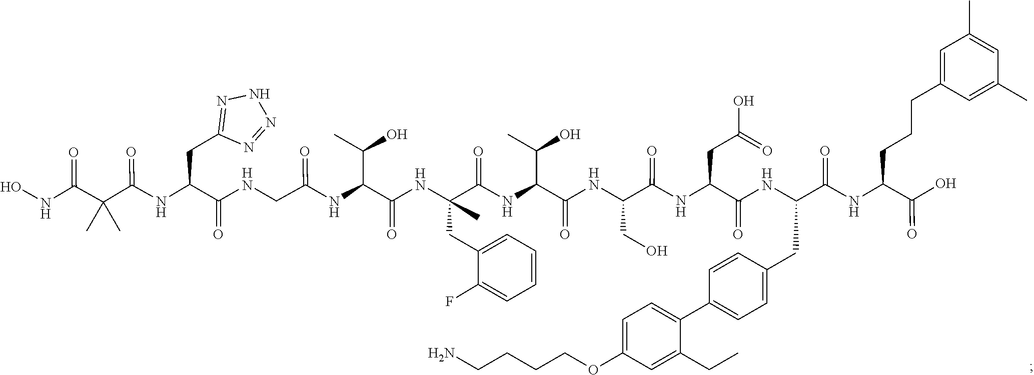 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NO)[C@@H](C)O)[C@@H](C)O)C(=O)N[C@@H](CCCc2cc(C)cc(C)c2)C(=O)O)cc1] |
| P6 (SEQ ID NO: 152) | 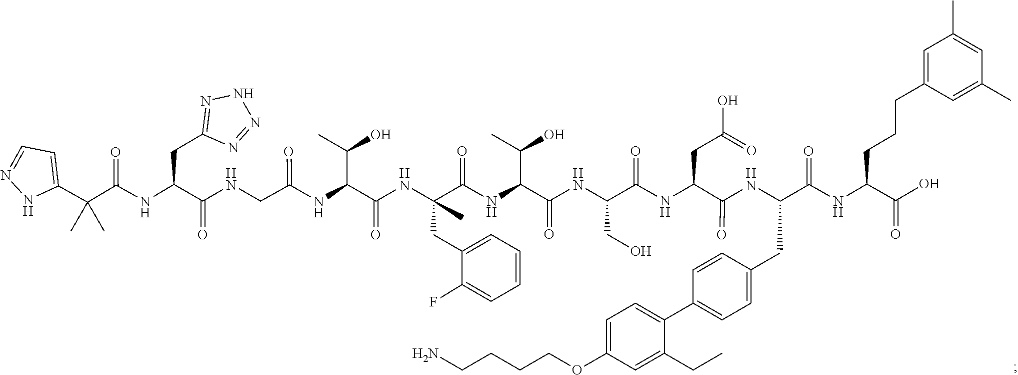 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)c2ccn[nH]2)[C@@H](C)O)[C@@H](C)O)C(=O)N[C@@H](CCCc2cc(C)cc(C)c2)C(=O)O)cc1] |
| P7 (SEQ ID NO: 153) | 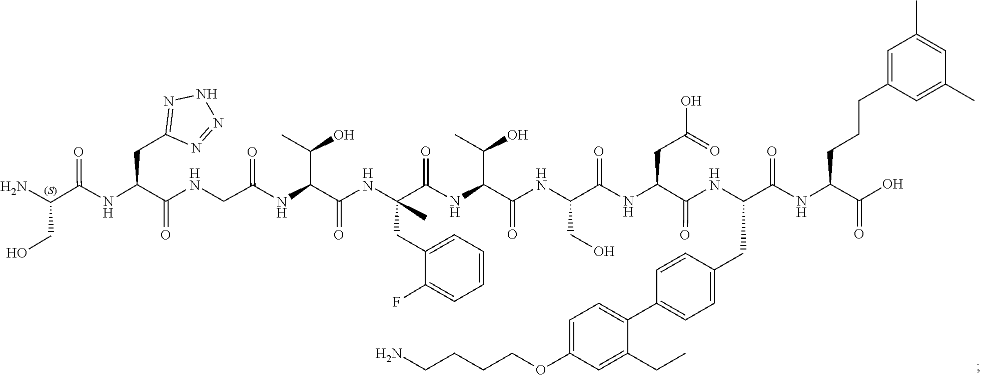 |
| P8 (SEQ ID NO: 154) | 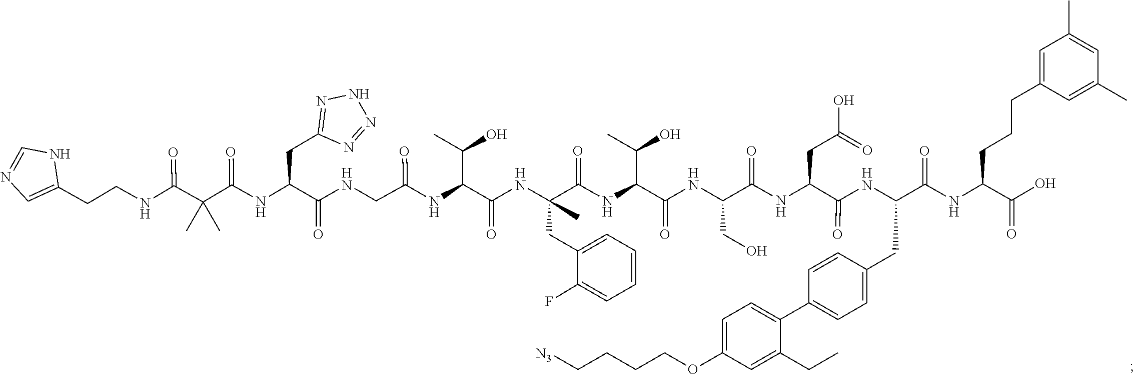 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| P9 (SEQ ID NO: 155) | 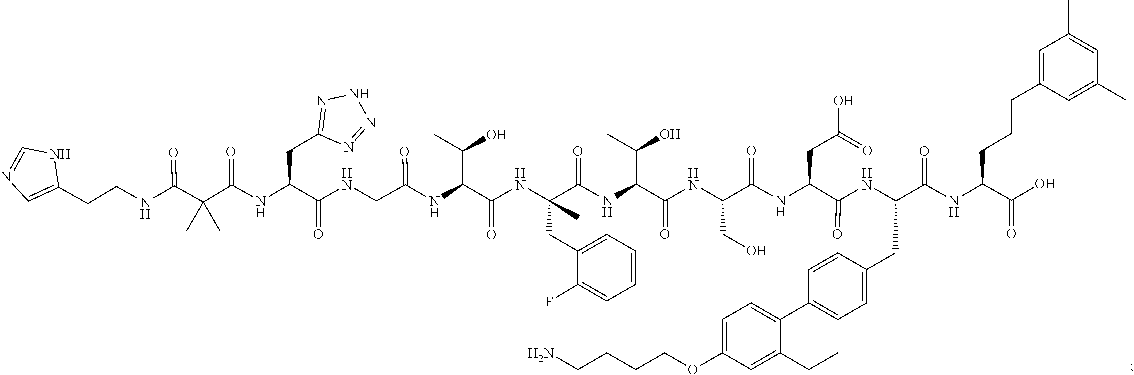 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| P10 (SEQ ID NO: 156) | 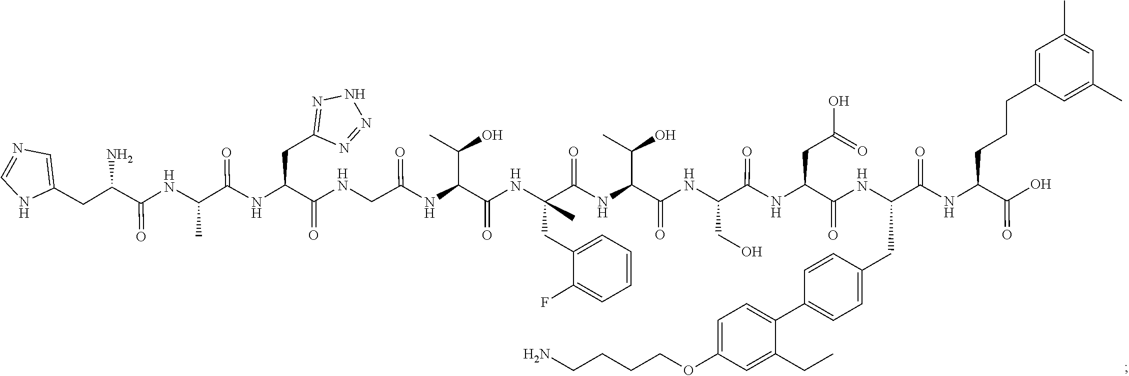 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)[C@H](C)NC(=O)[C@@H](N)Cc2cnc[nH]2)[C@@H](C)O)[C@@H](C)O)C(=O)N[C@@H](CCCc2cc(C)cc(C)c2)C(=O)O)cc1] |
| P11 (SEQ ID NO: 157) | 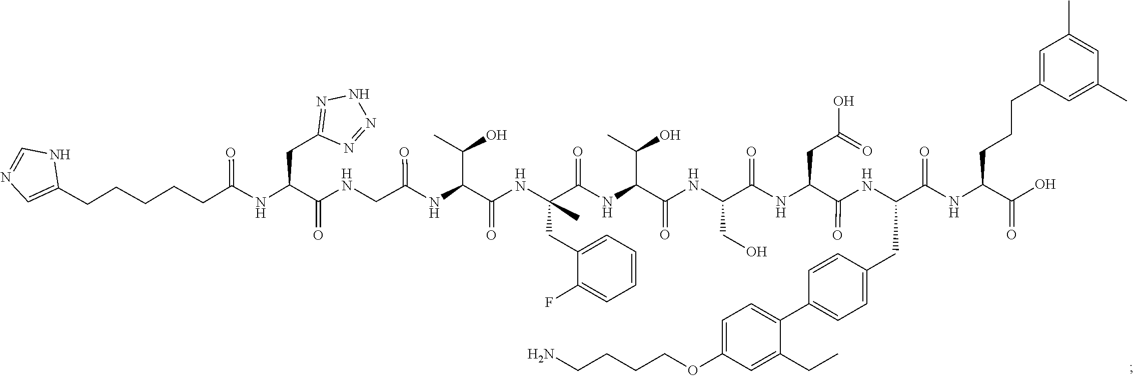 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(=O)O)cc1] |
| P12 (SEQ ID NO: 158) | 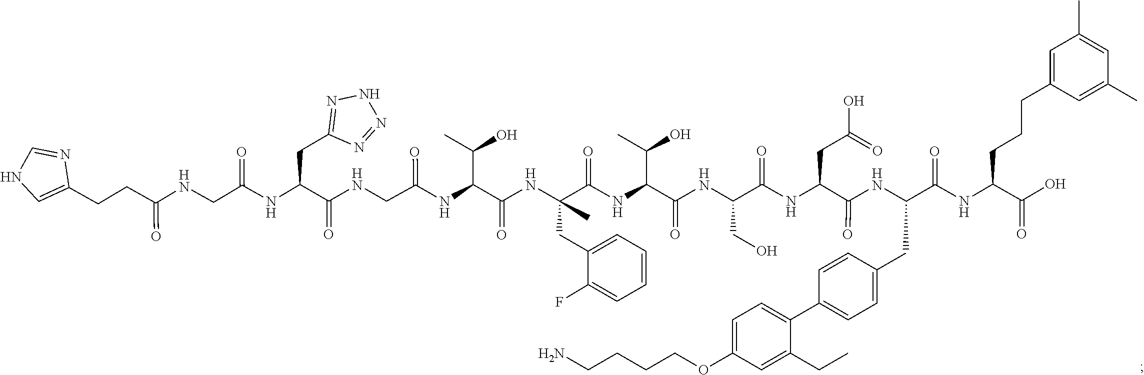 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CNC(=O)CCc2c[nH]cn2)[C@@H](C)O)[C@@H](C)O)C(=O)N[C@@H](CCCc2cc(C)cc(C)c2)C(=O)O)cc1] |
| P13 (SEQ ID NO: 159) | 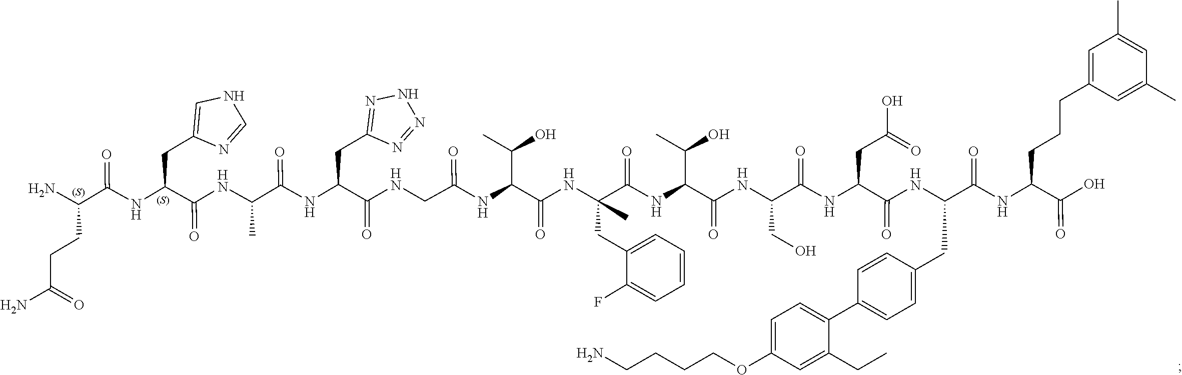 |
| P14 (SEQ ID NO: 160) | 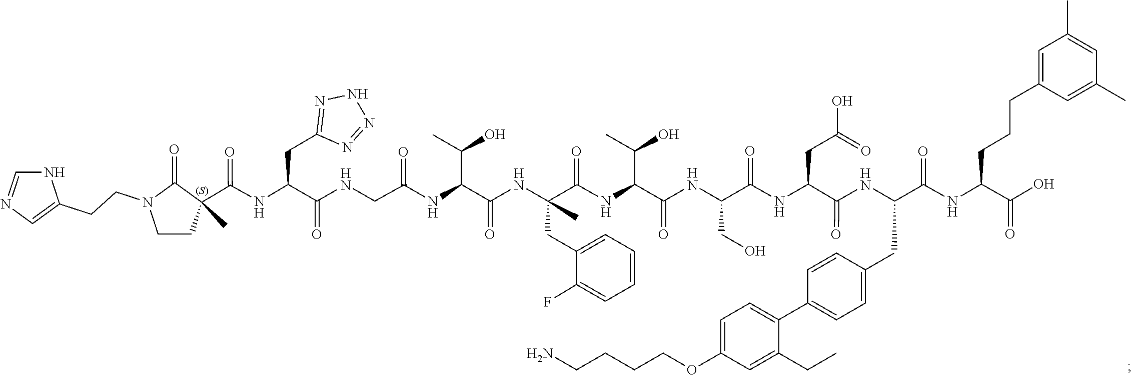 |
| P15 (SEQ ID NO: 161) | 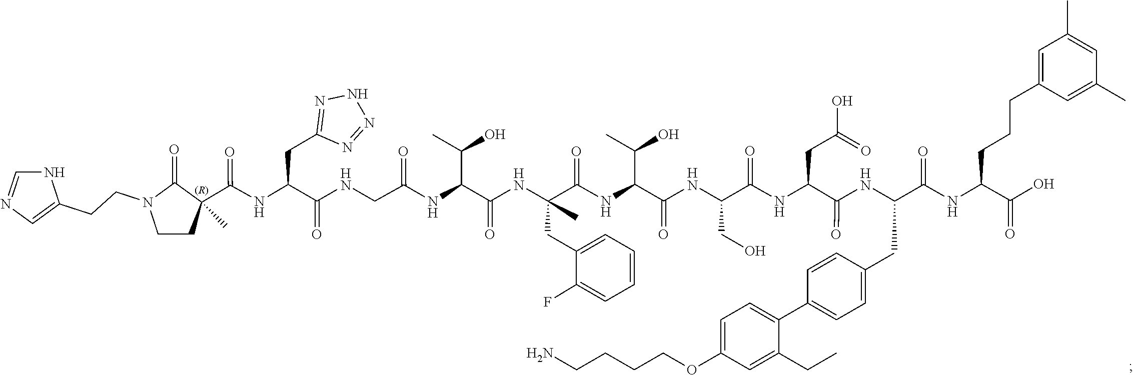 |
| P16 (SEQ ID NO: 162) | 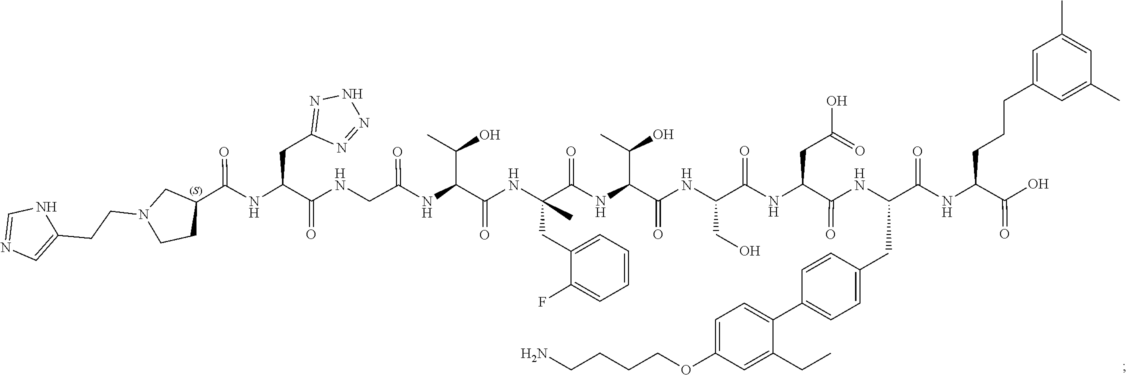 |
| P17 (SEQ ID NO: 163) | 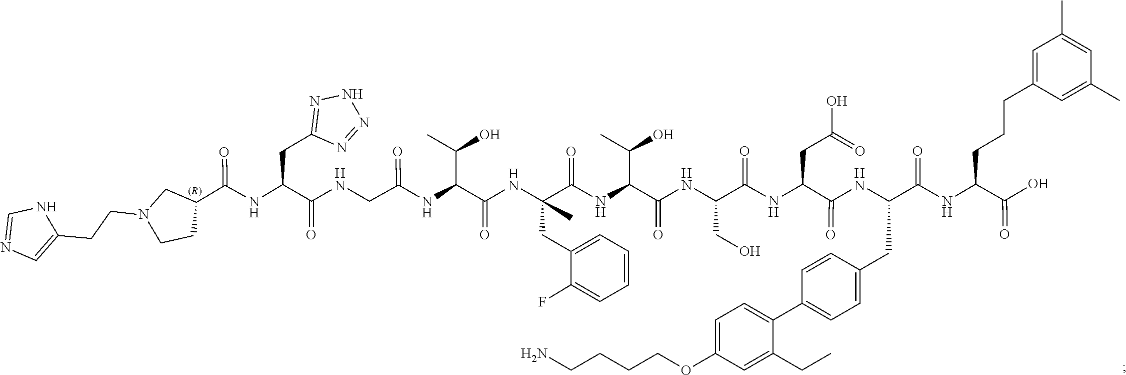 |
| P18 (SEQ ID NO: 164) | 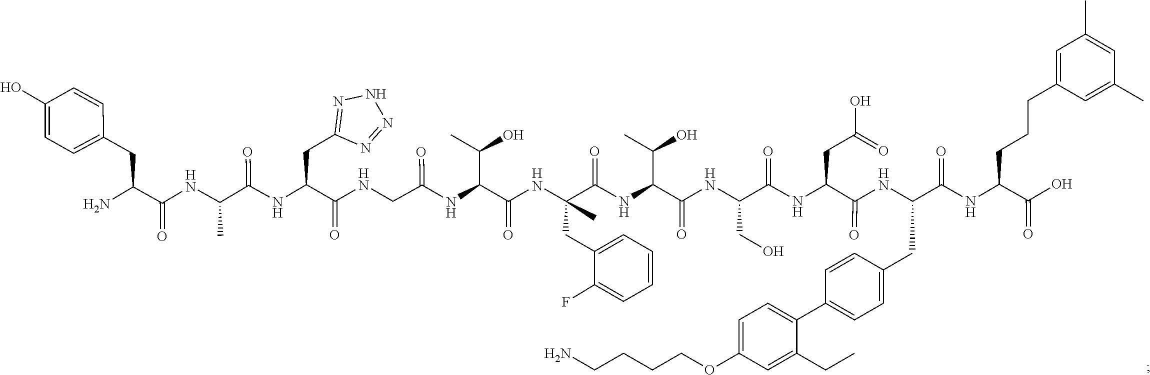 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)[C@H](C)NC(=O)[C@@H](N)Cc2ccc(O)cc2)[C@@H](C)O)[C@@H](C)O)C(=O)N[C@@H](CCCc2cc(C)cc(C)c2)C(=O)O)cc1] |
| P19 (SEQ ID NO: 165) | 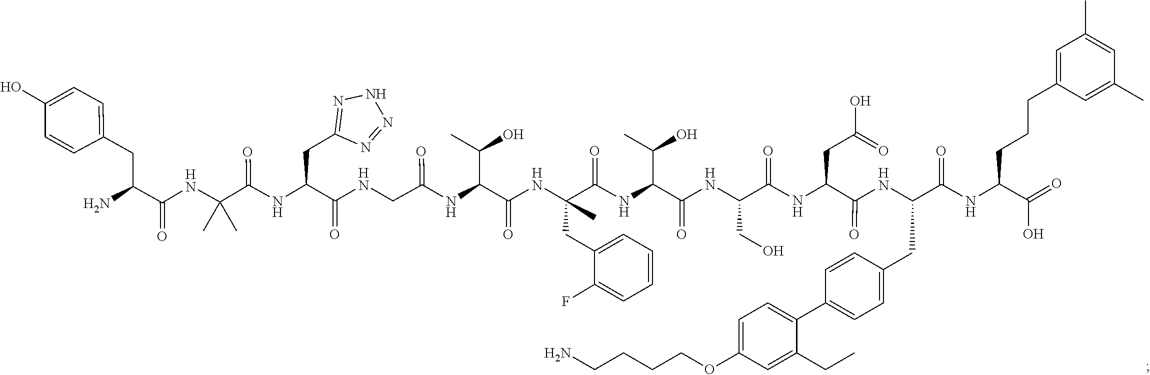 [SMILES: CCc1cc(OCCCCN)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)[C@@H](N)Cc2ccc(O)cc2)[C@@H](C)O)[C@@H](C)O)C(=O)N[C@@H](CCCc2cc(C)cc(C)c2)C(=O)O)cc1] |
| P20 (SEQ ID NO: 166) | 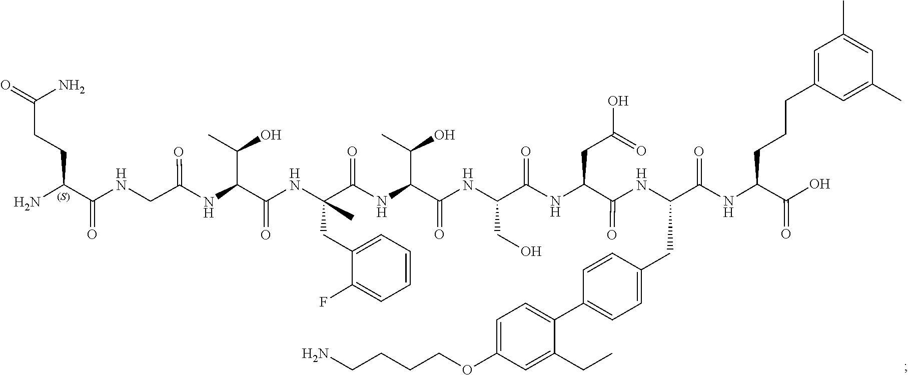 |
| P21 (SEQ ID NO: 167) | 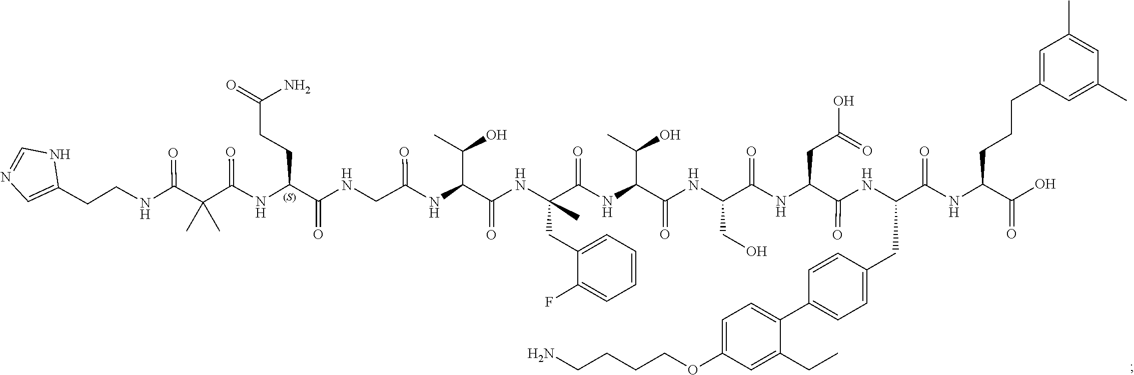 |
| P24 (SEQ ID NO: 168) | 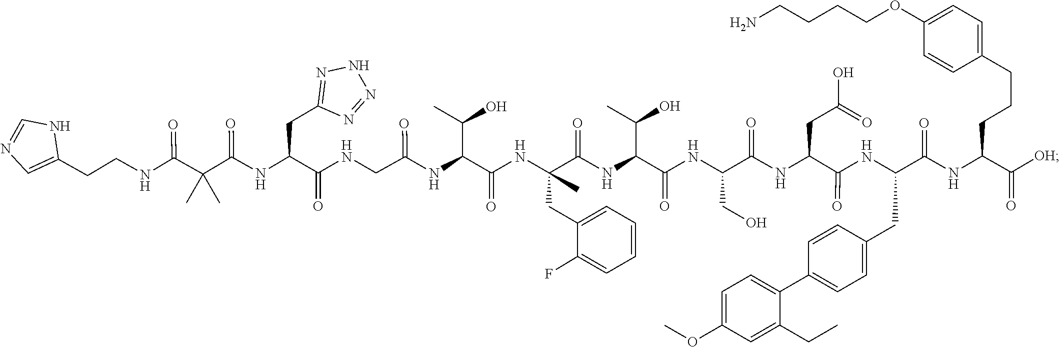 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCN)cc2)C(=O)O)cc1] |
| P25 (SEQ ID NO: 169) | 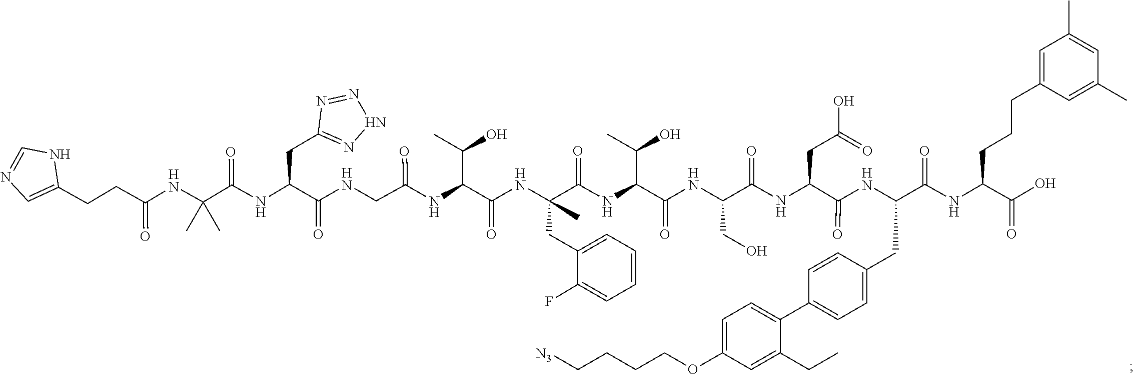 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCc2cnc[nH]2)[C@@H](C)O)[C@@H](C)O)C(=O)N[C@@H](CCCc2cc(C)cc(C)c2)C(=O)O)cc1] |
| P26 (SEQ ID NO: 170) | 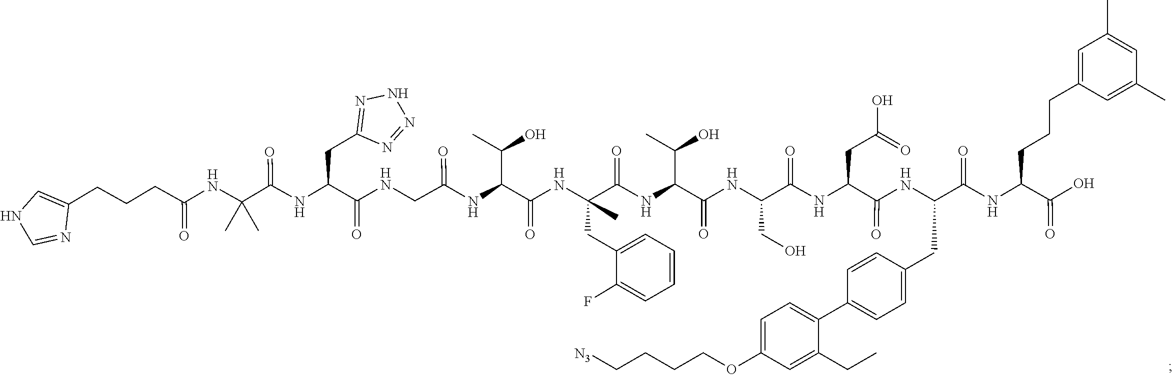 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCc2c[nH]cn2)[C@@H](C)O)[C@@H](C)O)C(=O)N[C@@H](CCCc2cc(C)cc(C)c2)C(=O)O)cc1] |
| P27 (SEQ ID NO: 171) | 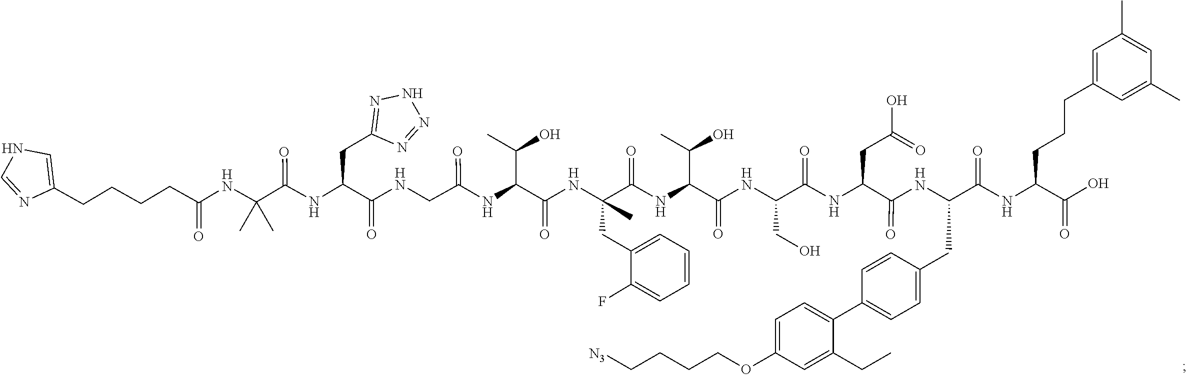 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCCc2c[nH]cn2)[C@@H](C)O)[C@@H](C)O)C(=O)N[C@@H](CCCc2cc(C)cc(C)c2)C(=O)O)cc1] |
| P28 (SEQ ID NO: 172) | 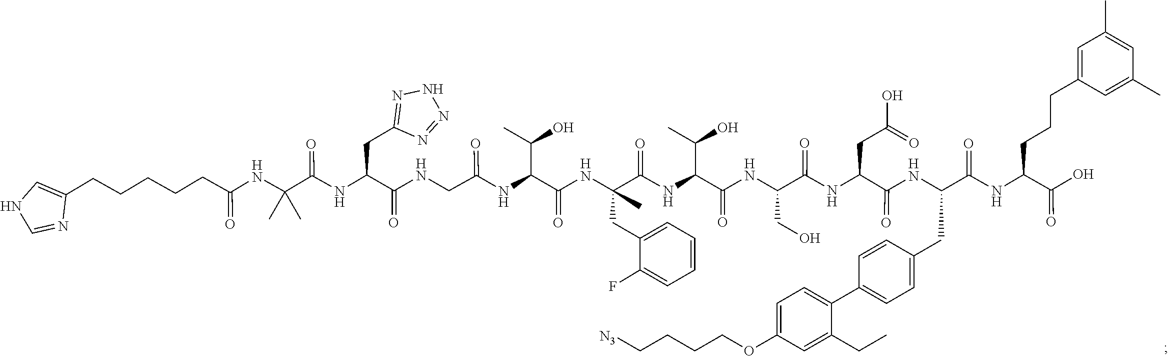 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCCCc2c[nH]cn2)[C@@H](C)O)[C@@H](C)O)C(=O)N[C@@H](CCCc2cc(C)cc(C)c2)C(=O)O)cc1] |
| P29 (SEQ ID NO: 173) | 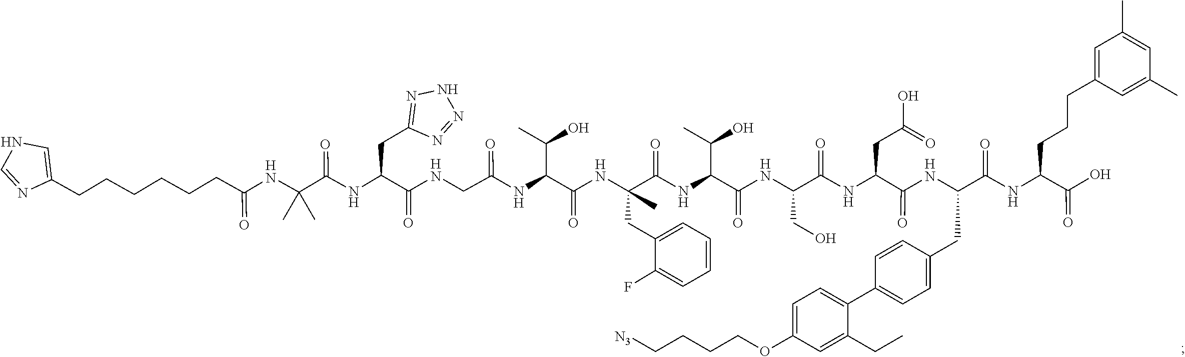 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)NC(=O)CCCCCCc2c[nH]cn2)[C@@H](C)O)[C@@H](C)O)C(=O)N[C@@H](CCCc2cc(C)cc(C)c2)C(=O)O)cc1] |
| P30 (SEQ ID NO: 174) | 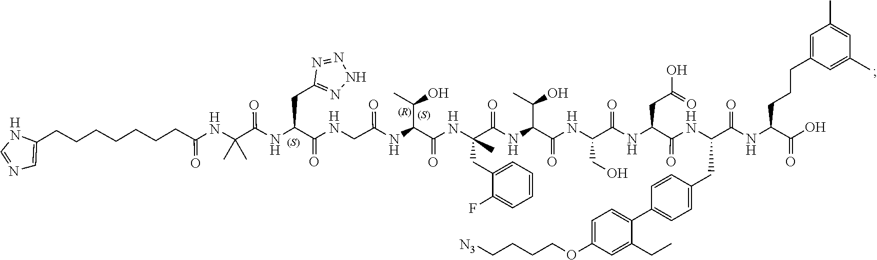 |
| P31 (SEQ ID NO: 175) | 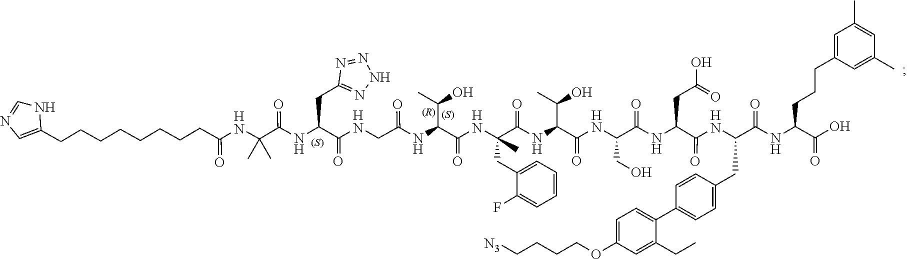 |
| P32 (SEQ ID NOS 176 and 177, respec- tively) | 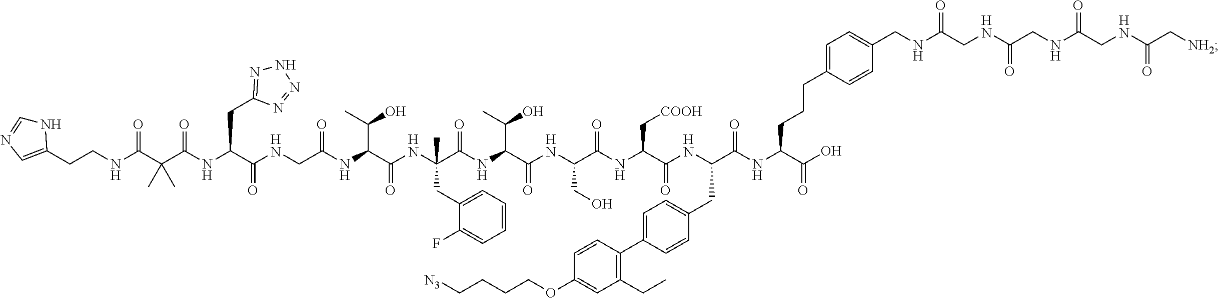 |
| P33 (SEQ ID NO: 178) | 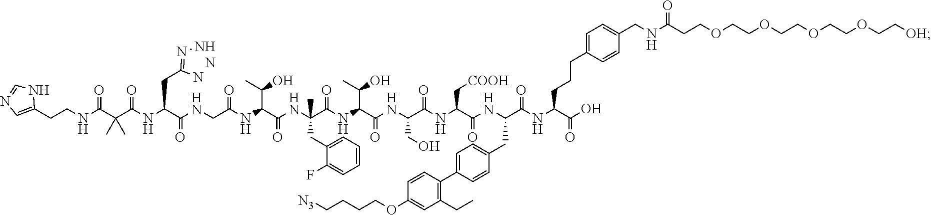 |
| P34 (SEQ ID NOS 179 and 180, respec- tively) | 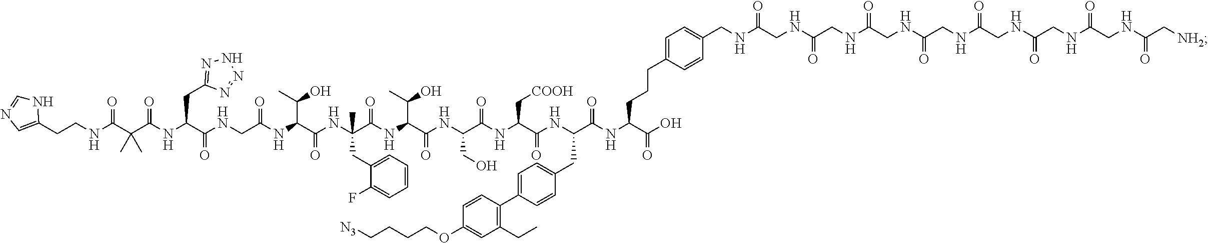 |
| P35 (SEQ ID NO: 181) | 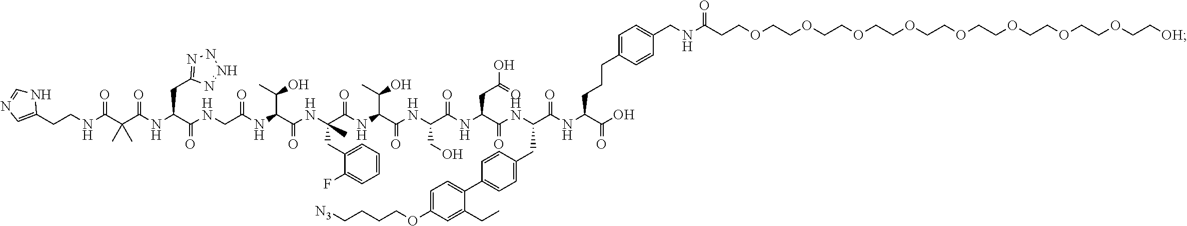 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(CNC(=O)CCOCCOCCOCCOCCOCCOCCOCCOCCO)cc2)C(=O)O)cc1] |
| P36 (SEQ ID NO: 182) | 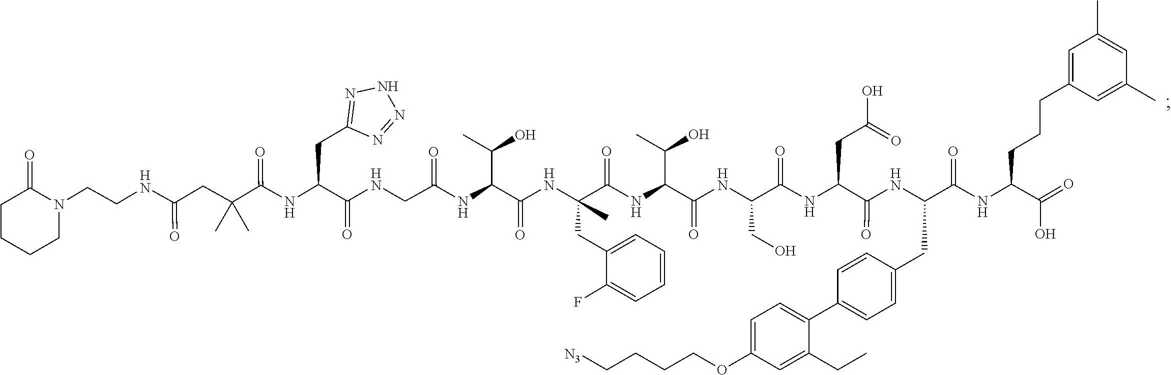 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)CC(=O)NCCN2CCCCC2=O)[C@@H](C)O)[C@@H](C)O)C(=O)N[C@@H](CCCc2cc(C)cc(C)c2)C(=O)O)cc1] |
| P37 (SEQ ID NO: 183) | 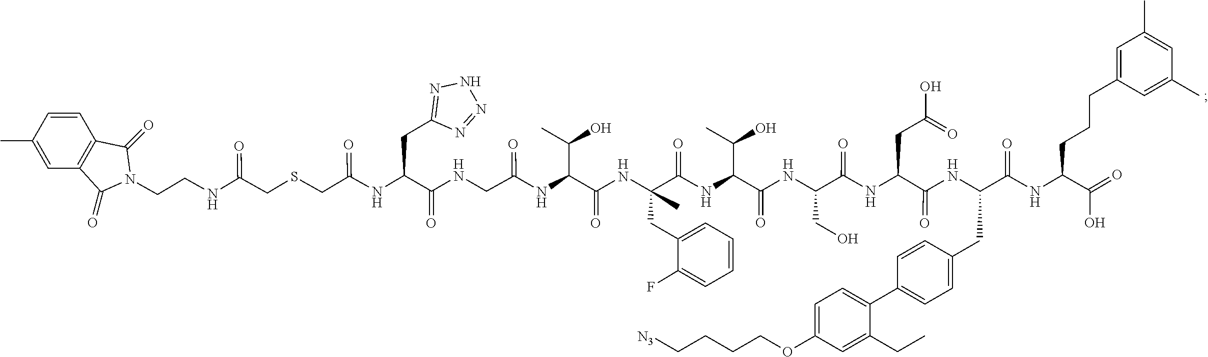 |
| P38 (SEQ ID NO: 184) | 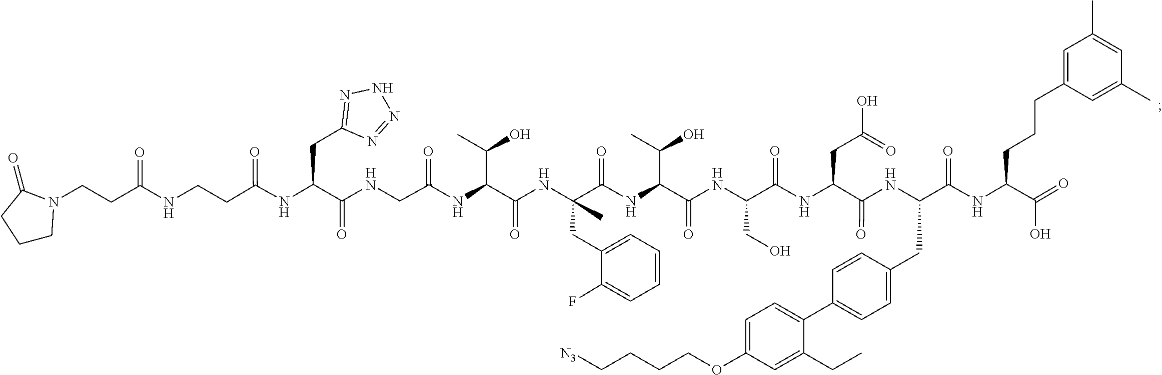 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCNC(=O)CCN2CCCC2=O)[C@@H](C)O)[C@@H](C)O)C(=O)N[C@@H](CCCc2cc(C)cc(C)c2)C(=O)O)cc1] |
| P39 (SEQ ID NO: 185) | 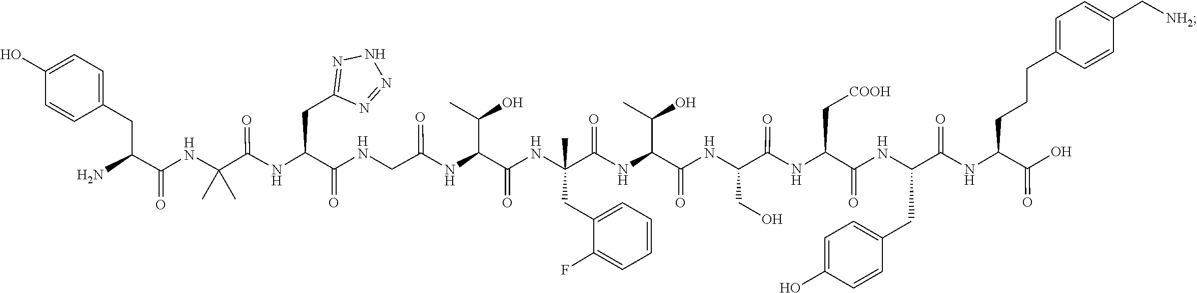 |
| P40 (SEQ ID NO: 186) | 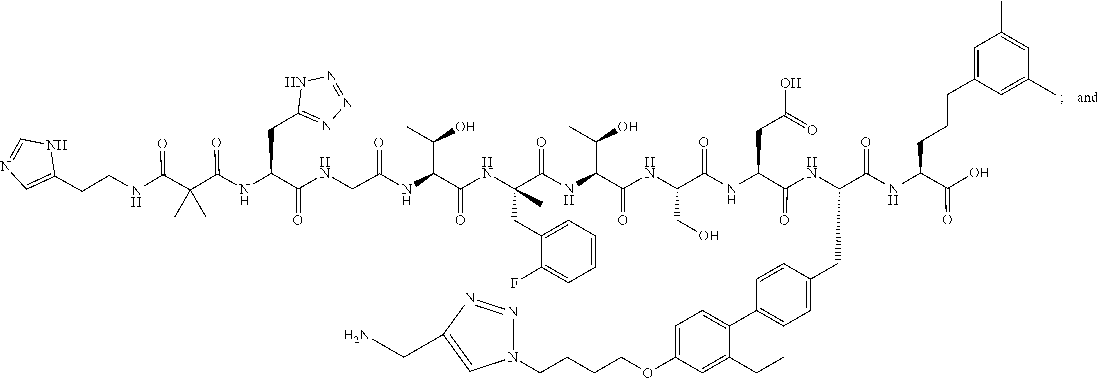 [SMILES: CCc1cc(OCCCCn2cc(CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| P42 (SEQ ID NO: 187) | 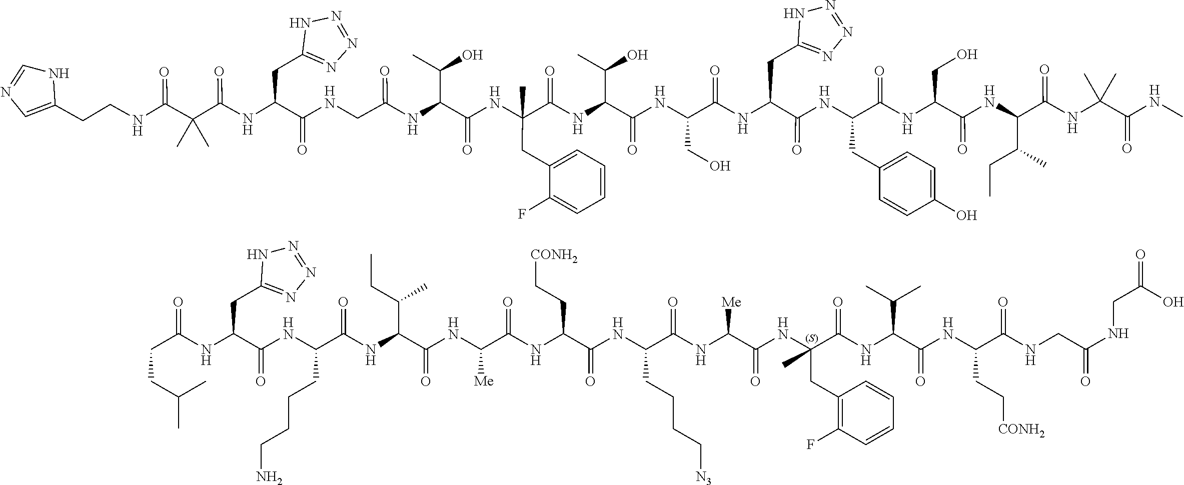 |

In another embodiment, the payloads P according to the present disclosure have a structure selected from:

| P# | Structure |
|---|---|
| P43 (SEQ ID NO: 188) | 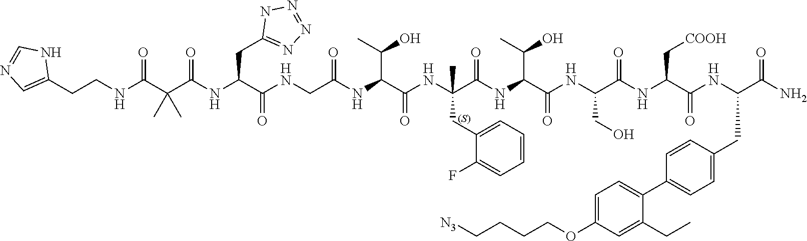 |
| P44 (SEQ ID NO: 189) | 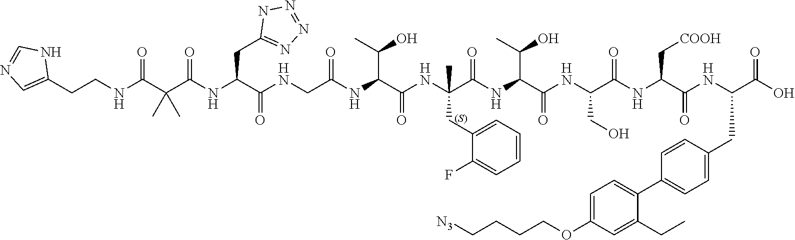 |
| P45 (SEQ ID NO: 190) | 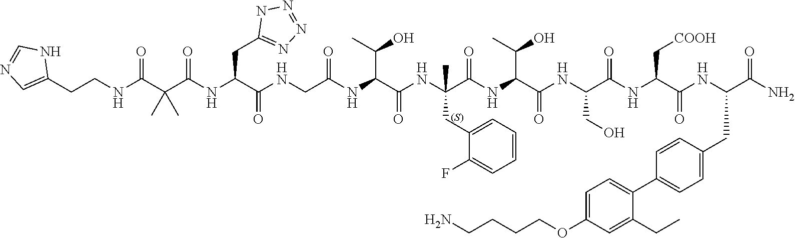 |
| P46 (SEQ ID NO: 191) | 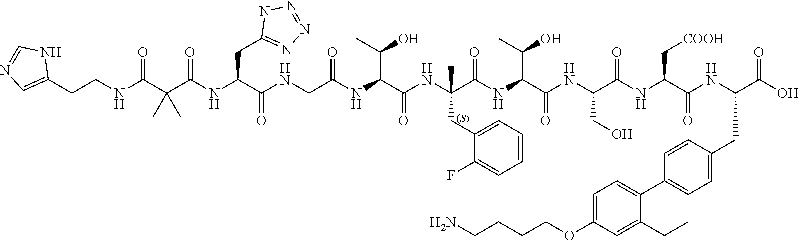 |
| P47 (SEQ ID NO: 192) | 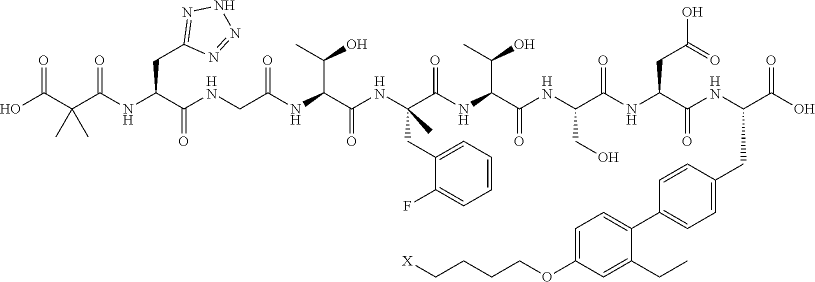 |
| | X = —NH₂ or —N₃. |
| P48 (SEQ ID NO: 193) | 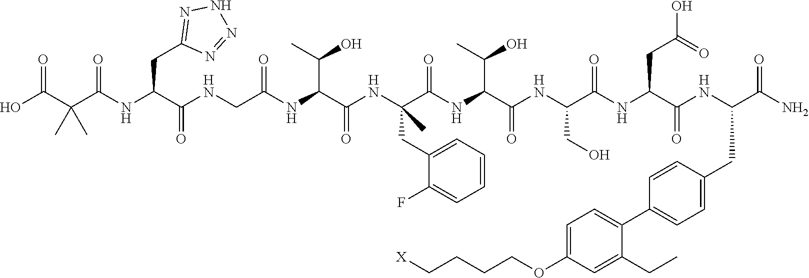 |
| | X = —NH₂ or —N₃. |
| P49 (SEQ ID NO: 194) | 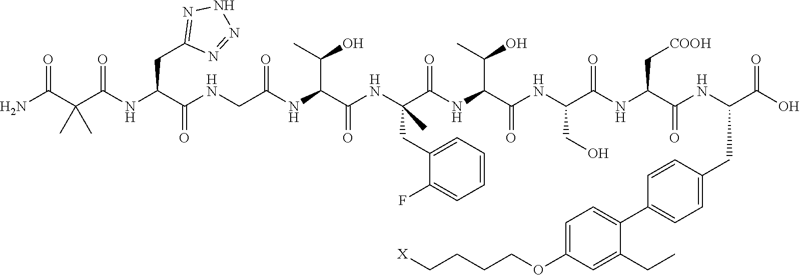 |
| | X = —NH₂ or —N₃. |
| P50 (SEQ ID NO: 195) | 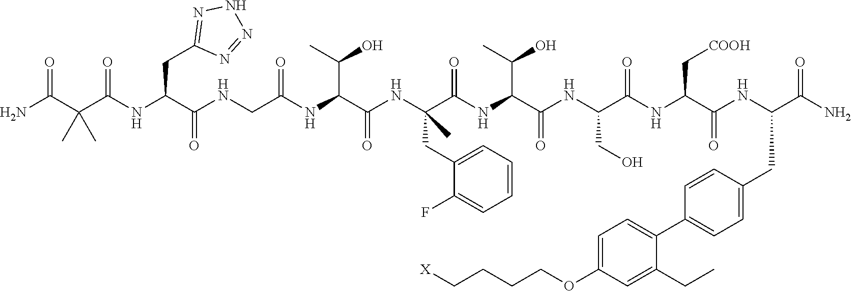 |
| | X = —NH₂ or —N₃ |
| P51 (SEQ ID NO: 196) | 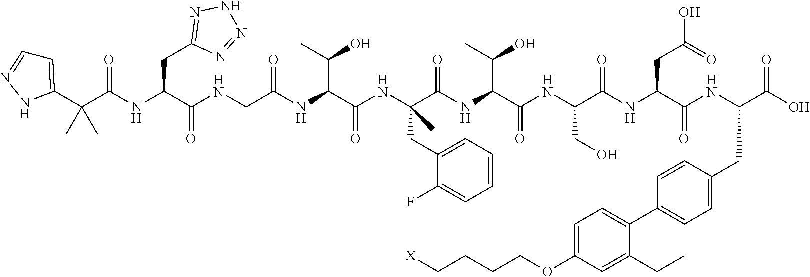 |
| | X = —NH₂ or —N₃ |
| P52 (SEQ ID NO: 197) | 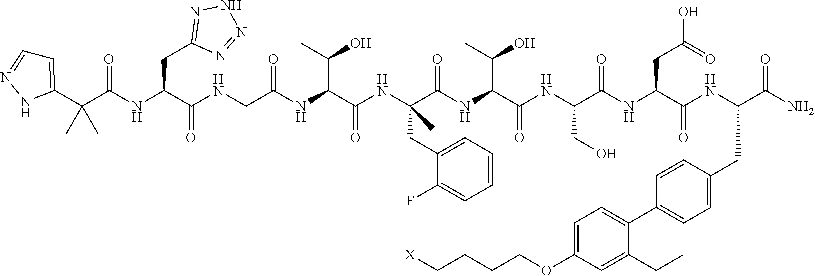 |
| | X = —NH₂ or —N₃ |
| P53 (SEQ ID NO: 198) | 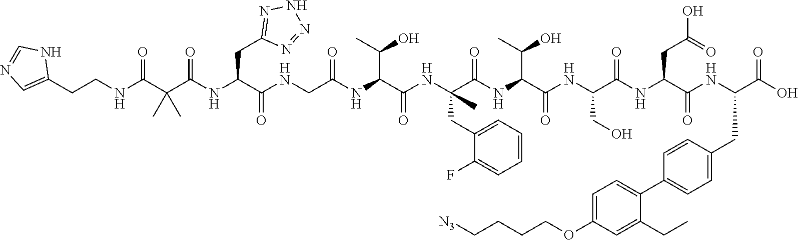 [SMILES: CCc1cc(OCCCCN=[N+]=[N-])ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| | X = —NH₂ or —N₃ |
| P54 (SEQ ID NO: 199) | 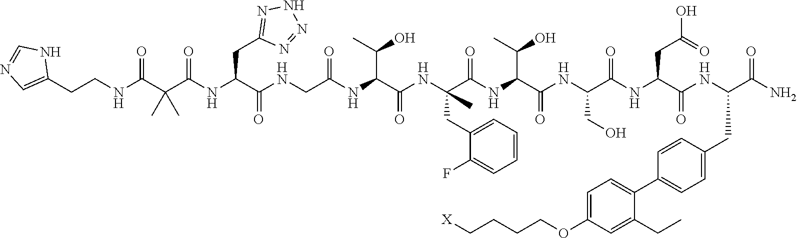 |
| | X = —NH₂ or —N₃ |
| P55 (SEQ ID NO: 200) | 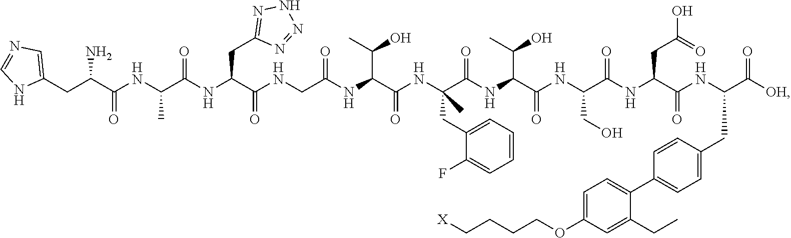 |
| | X = —NH₂ or —N₃ |
| P56 (SEQ ID NO: 201) | 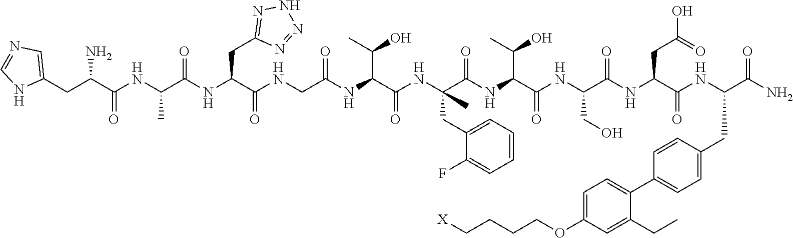 |
| | X = —NH₂ or —N₃ |
| P57 (SEQ ID NO: 202) | 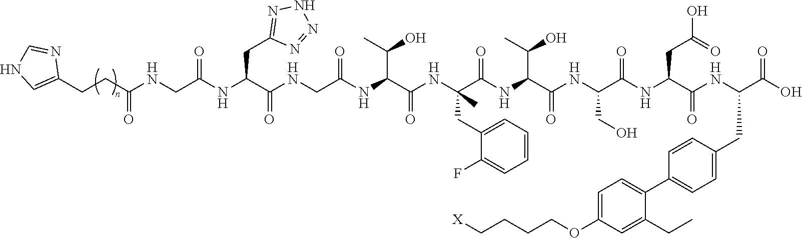 |
| | n is an integer of 1~7; X = —NH₂ or —N₃. |
| P58 (SEQ ID NO: 203) | 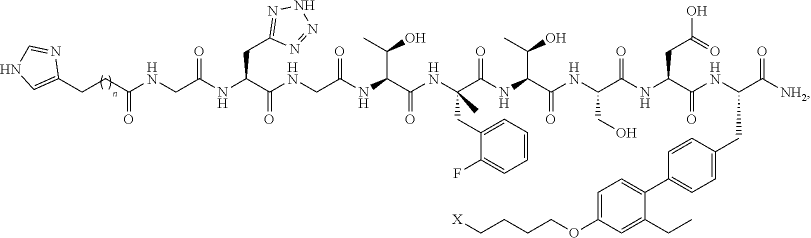 |
| | n is an integer of 1~7; X = —NH₂ or —N₃. |
| P59 (SEQ ID NO: 204) | 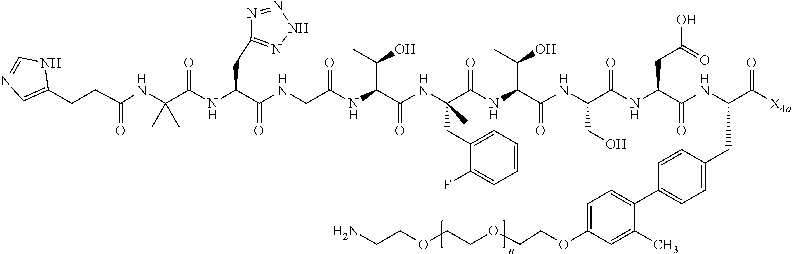 |
| | X₄ₐ is —OH or —NH₂; n is an integer of one to twelve; |
| P60 (SEQ ID NO: 205) | 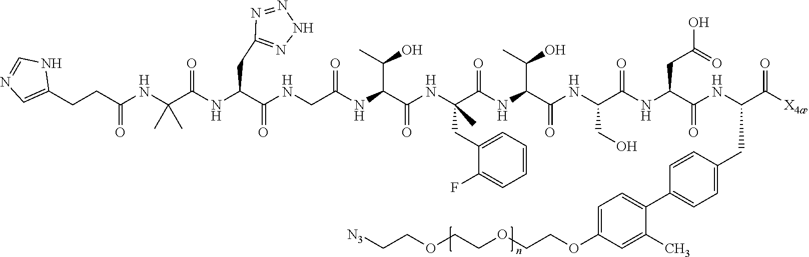 |
| | X₄ₐ is —OH or —NH₂; n is an integer of one to twelve; |

or a pharmaceutically acceptable salt thereof.

In some embodiments, the payloads of the present disclosure are amenable to conjugation with a binding agent (e.g., antibody).

In one embodiment, the payloads of the present disclosure are conjugated to an antibody or an antigen-binding fragment thereof directly.

In another embodiment, the payloads of the present disclosure are conjugated to an antibody or an antigen-binding fragment thereof through a non-cleavable linker. Any of the linkers of the present disclosure can be used to attach the payloads of the present disclosure to the antibody or antigen-binding fragment thereof.

### Linker-Payloads (L-P)

In one embodiment, the present disclosure provides reactive linker-payloads comprising payloads P as described above and linkers capable of covalently attaching to an antibody or an antigen-binding fragment thereof.

In one embodiment, the linker-payload according to the present disclosure has a structure of Formula (LP-I), Formula (LP-II), Formula (LP-II′), or Formula (LP-III):

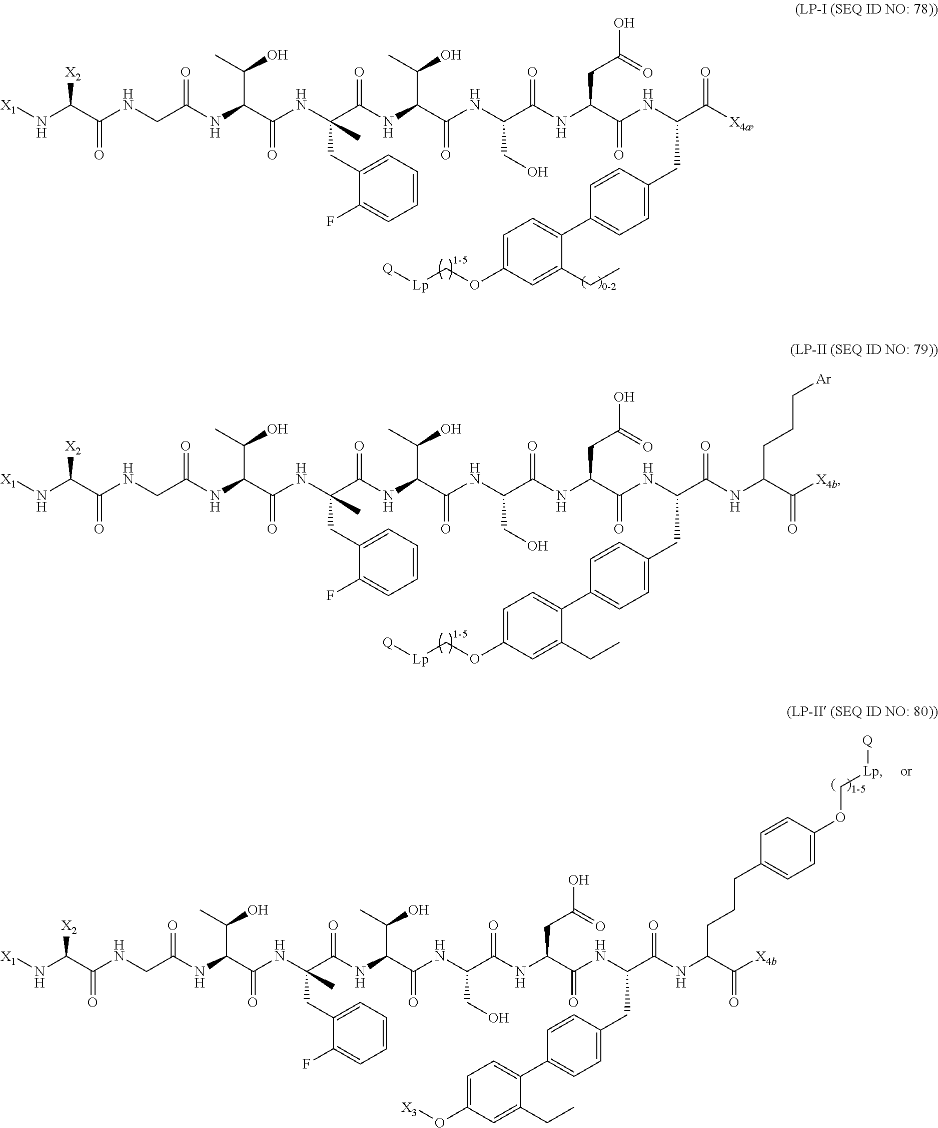

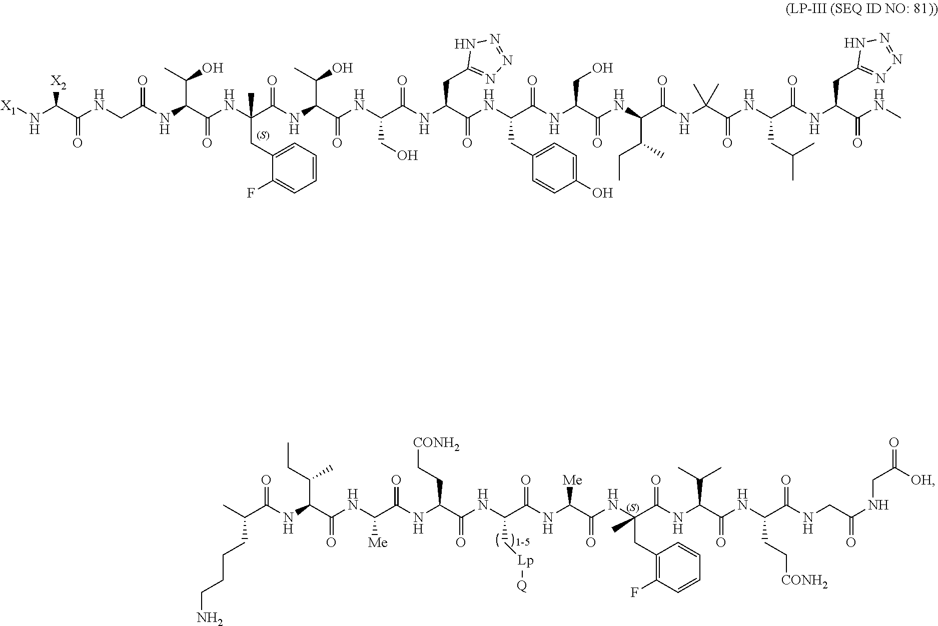

wherein:
X₁ is selected from H;

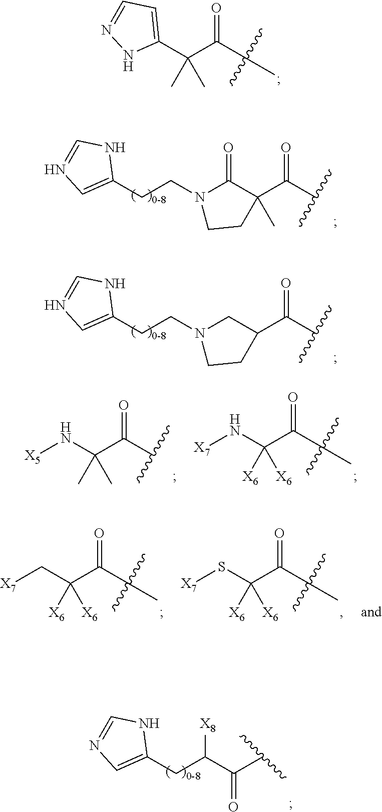

X₂ is selected from

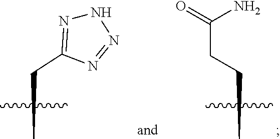

X₃ is selected from —CH₃, —(CH₂)₂₋₆—NH₂, —(CH₂)₂₋₆—N₃, and —(CH₂)₂₋₆-Tr-(CH₂)₁₋₆—NH₂, where
Tr is a triazole moiety;
Lₚ is absent or a linker comprising one or more of

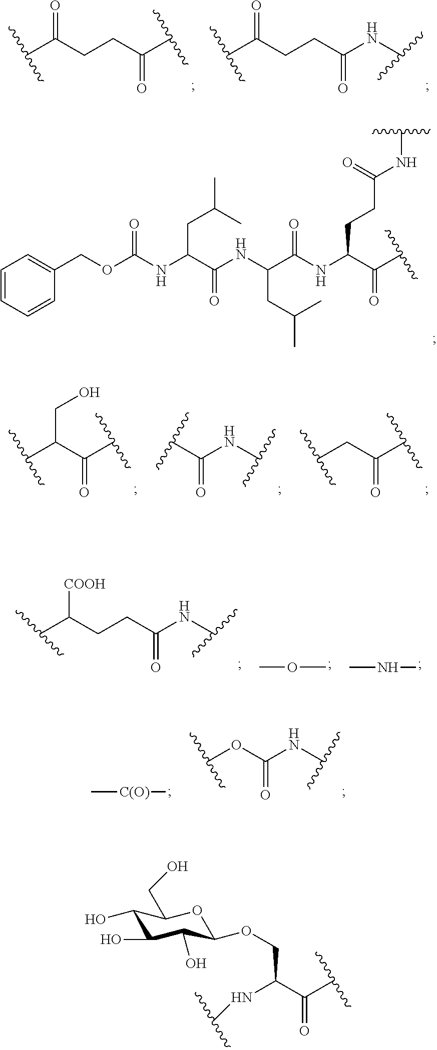

a cyclodextrin; —CH₂—O—; a polyethylene glycol (PEG) segment having 1 to 36-CH₂CH₂O-(EG) units; —(CH₂)₁₋₂₄-; a triazole; one or more amino acids selected from glycine, serine, glutamic acid, alanine, valine, threonine, leucine, and proline, and combinations thereof;
Q is a moiety selected from

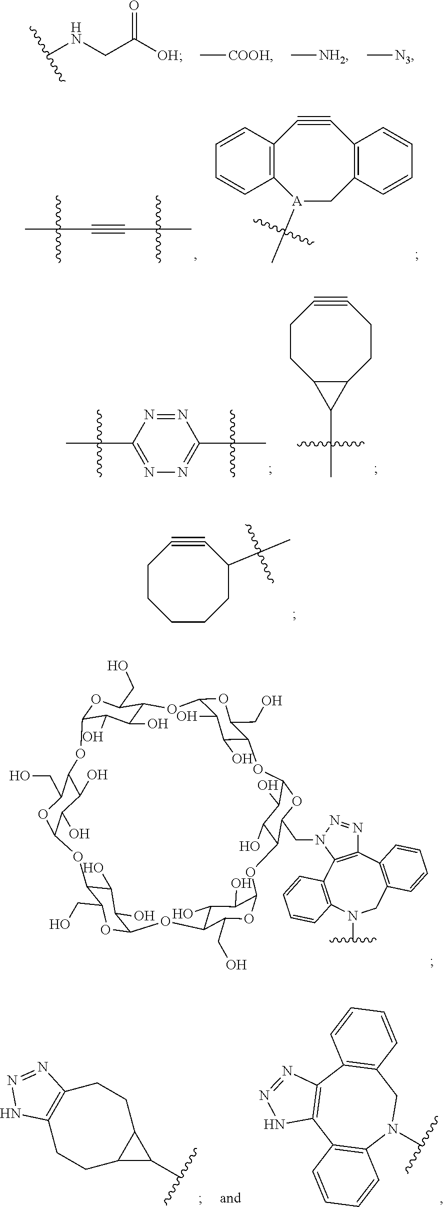

where A is C or N;
X₄ₐ is —NH₂ or —OH;
X_{4b} is —OH;
X₅ is selected from —OH, —NH₂, —NH—OH, and

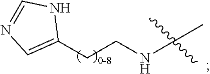

X₆ is independently at each occurrence selected from H, —OH, —CH₃, and —CH₂OH;
X₇ is selected from H,

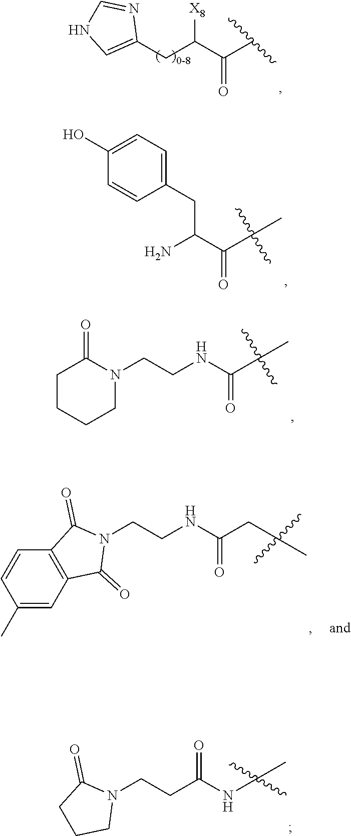

X₈ is selected from H, —OH, —NH₂, and

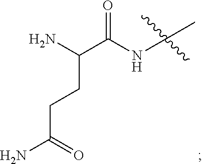 [SMILES: *NC(=O)C(N)CCC(N)=O]

Ar is selected from

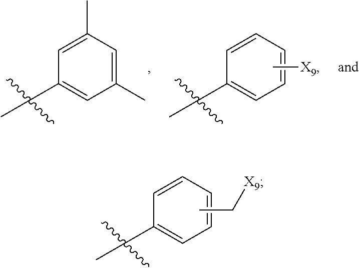

and
X₉ is selected from the group consisting of-NH₂,

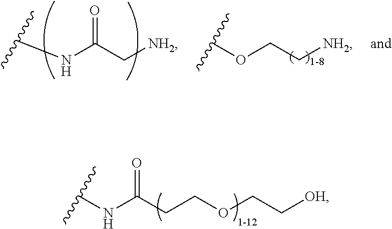

or a pharmaceutically acceptable salt thereof,
with the proviso that when X_{4b} is —OH, Ar is

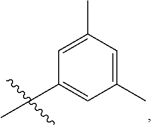 [SMILES: *c1cc(C)cc(C)c1]

### Lp is

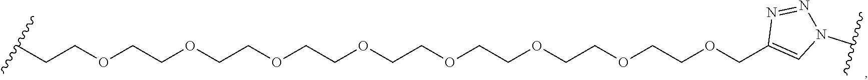

### and Q is NH₂,

X₅ is not

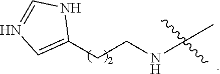

In some embodiments, X₁ is

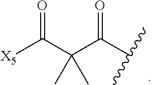

In some embodiments, X₂ is

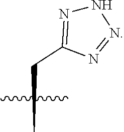

In some embodiments, X₃ is —(CH₂)₂₋₆—N₃.

In some embodiments, X₅ is

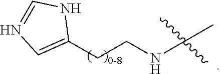

In some embodiments, X₄ₐ is —NH₂.

In some embodiments, X₄ₐ is-OH.

In some embodiments, the compound of Formula (LP-I) has a structure of Formula LP-Ia):

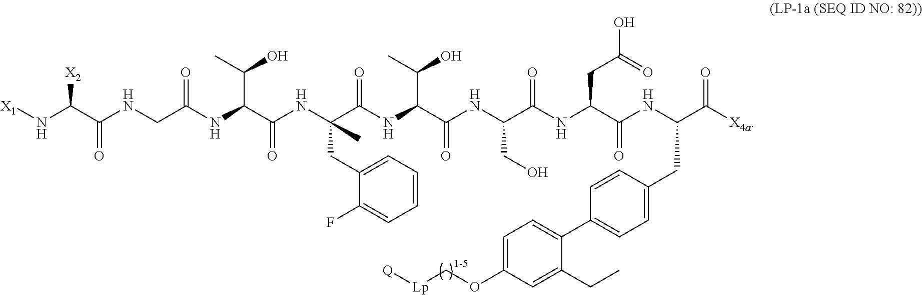

In some embodiments, the Lp comprises a polyethylene glycol (PEG) segment having 1 to 36-CH₂CH₂O-(EG) units. In some embodiments, the PEG segment comprises between 2 and 30 EG units, between 4 and 24 EG units, or between 4 and 8 EG units. In some embodiments, the PEG segment comprises 1 EG unit, 2 EG units, 3 EG units, 4 EG units, 5 EG units, 6 EG units, 7 EG units, 8 EG units, 9 EG units, 10 EG units, 11 EG units, or 12 EG units. In some embodiments, the PEG segment comprises 24 EG units. In some embodiments, the PEG segment comprises 4 EG units. In some embodiments, the PEG segment comprises 8 EG units.

In another embodiment, the Lp, comprises one or more amino acids selected from glycine, threonine, serine, glutamine, glutamic acid, alanine, valine, leucine, and proline and combinations thereof. In some embodiments, the Lp comprises 1 to 10 glycines, or 1 glycine, or 2 glycines, or 3 glycines, or 4 glycines, or 5 glycines, or 6 glycines, or 7 glycines, or 8 glycines, or 9 glycines, or 10 glycines. In some embodiments, the Lp comprises 1 to 6 serines, or 1 serine, or 2 serines, or 3 serines, or 4 serines, or 5 serines, or 6 serines. In some embodiments, the Lp comprises 1 to 10 glycines and 1 to 6 serines. In some embodiments, the Lp comprises 4 glycines and 1 serine.

In some embodiments, the Lp comprises a combination of a PEG segment having 1 to 36 EG units and one or more amino acids selected from glycine, serine, glutamic acid, alanine, valine, threonine, leucine, and proline, and combinations thereof.

In some embodiments, the serine residue comprises a carbohydrate group, e.g., a glucose group.

In some embodiments, the serine residue comprises a glucose group.

In some embodiments, the Lp comprises a combination of a PEG segment having 1 to 36 EG units and a triazole group.

In some embodiments, the Lp comprises a combination of a PEG segment having 1 to 36 EG units; a triazole group; and —CH₂—O— group.

In some embodiments, the Lp comprises a combination of a PEG segment having 1 to 36 EG units; a triazole group; —CH₂—O— group; and —(CH₂)₁₋₂₄— group.

In some embodiments, the Lp comprises a combination of a PEG segment having 5 to 10 EG units; a triazole group; —CH₂—O— group; and —(CH₂)₁₋₅— group.

In some embodiments, the Lp is

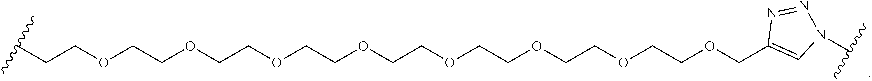

In one embodiment, the linker-payload LP comprises a cyclodextrin moiety. In some embodiments, the linker-payload LP comprising a cyclodextrin moiety exhibits GLP1R agonism activity.

In one embodiment, the linker-payloads LP according to the present disclosure have the structure selected from the group consisting of:

| LP# | Name | Structure |
|---|---|---|
| LP1 (SEQ ID NO: 84) | DIBAC- suc- PEG4- P9 | 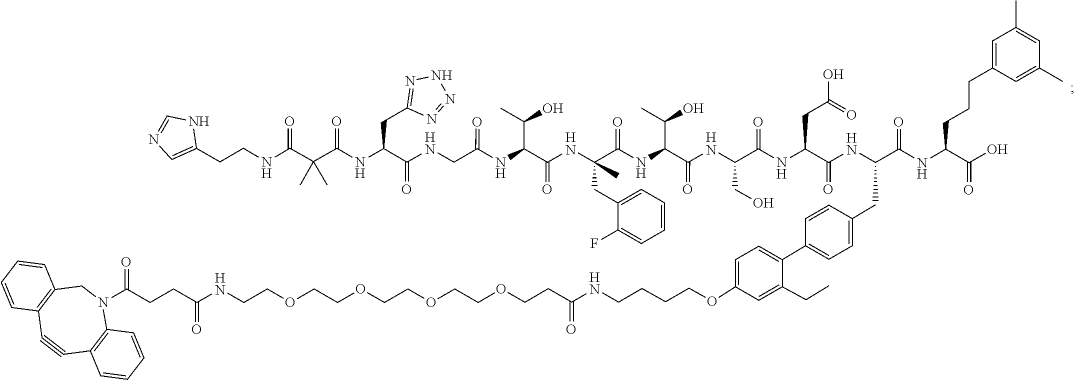 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP2 (SEQ ID NO: 85) | DIBAC- suc- PEG8- P9 | 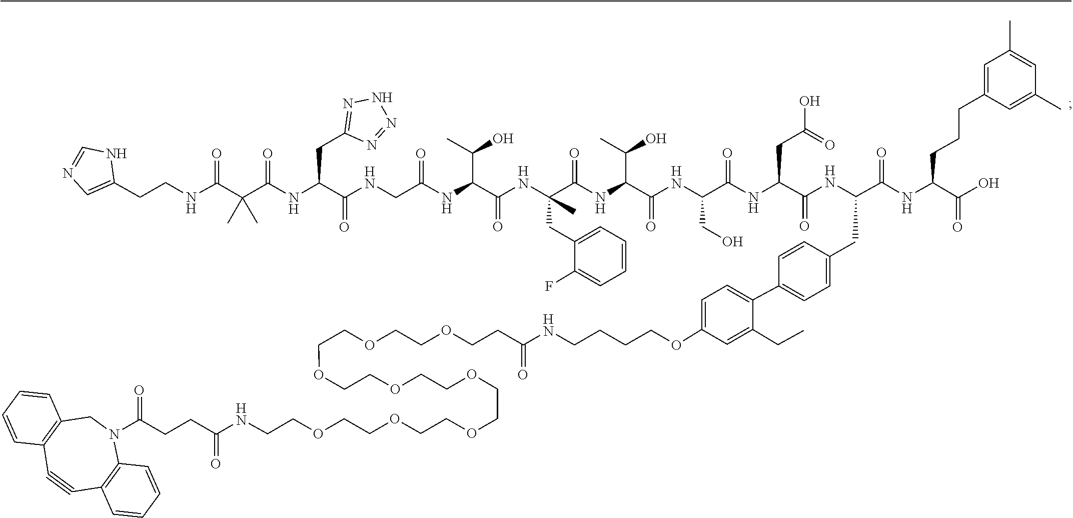 |
| LP3 (SEQ ID NO: 86) | DIBAC- suc- PEG12- P9 | 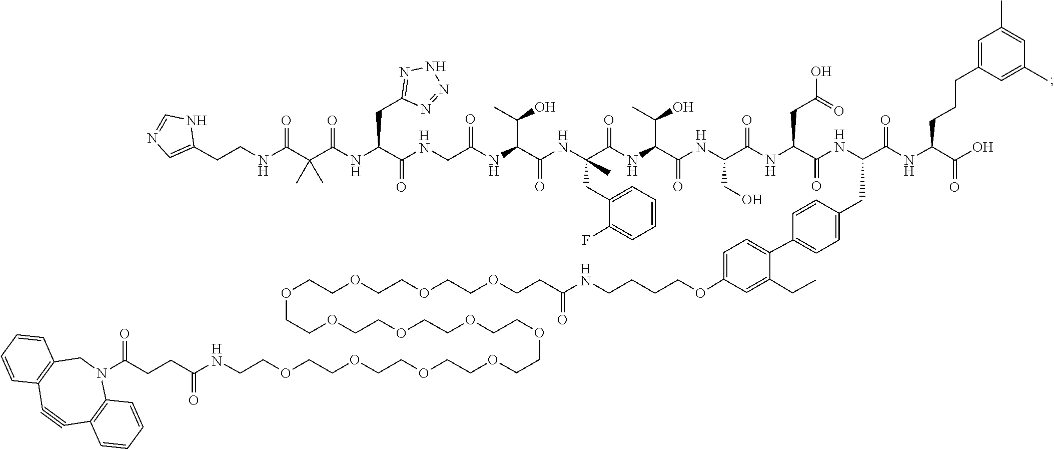 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP4 (SEQ ID NO: 87) | DIBAC- suc- PEG24 P9 | 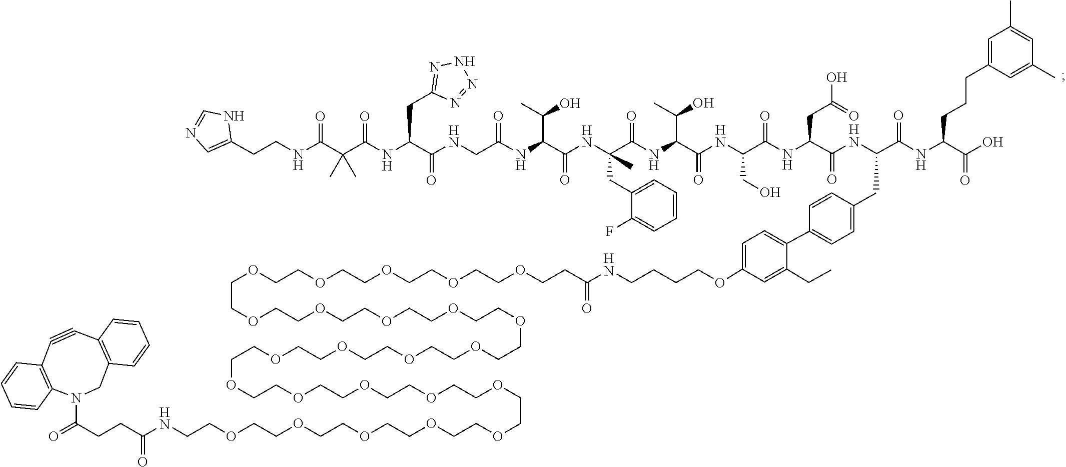 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP5 (SEQ ID NO: 88) | BCN- PEG4- carbamate- P9 | 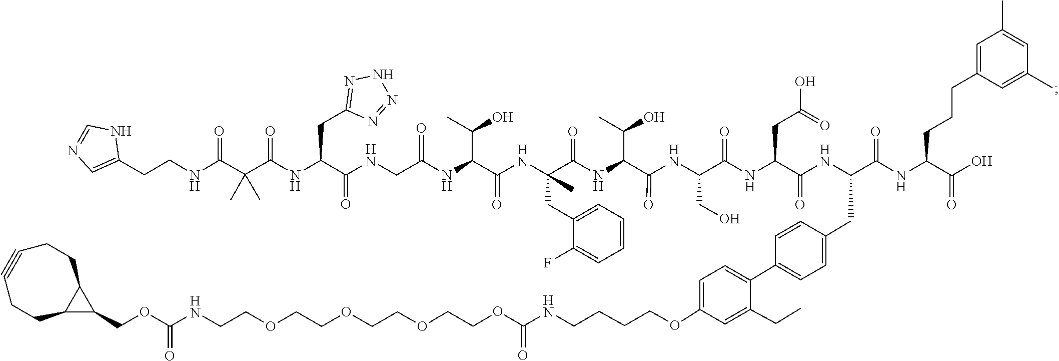 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP6 (SEQ ID NOS 89 and 90, respec- tively) | DIBAC- suc- G4S- P9 | 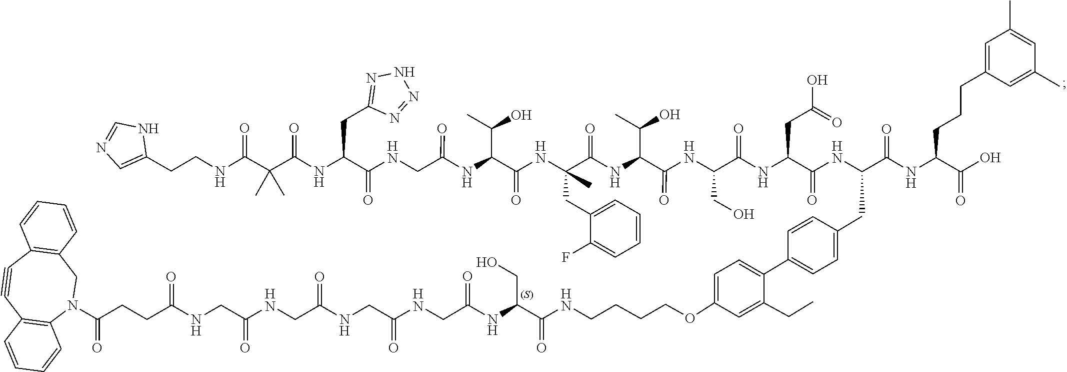 |
| LP7 (SEQ ID NOS 91 and 92, respec- tively) | DIBAC- suc- SG4- P9 | 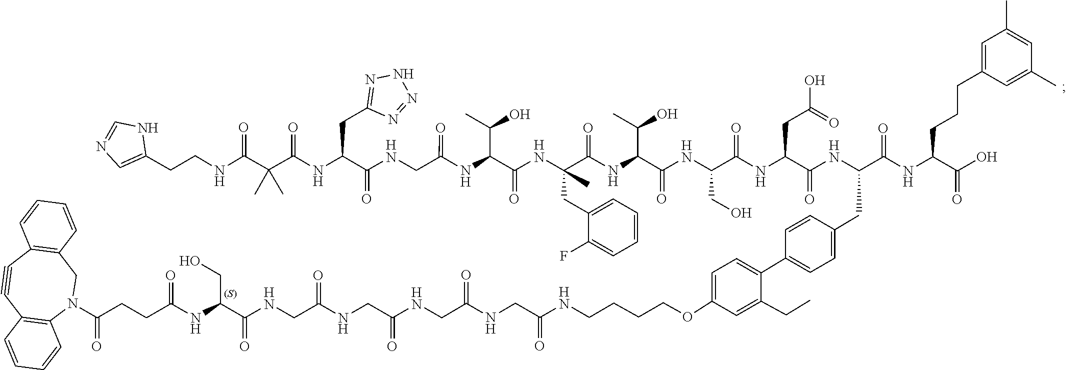 |
| LP8 (SEQ ID NO: 351) | DIBAC- suc- PEG4- triazole- P8 | 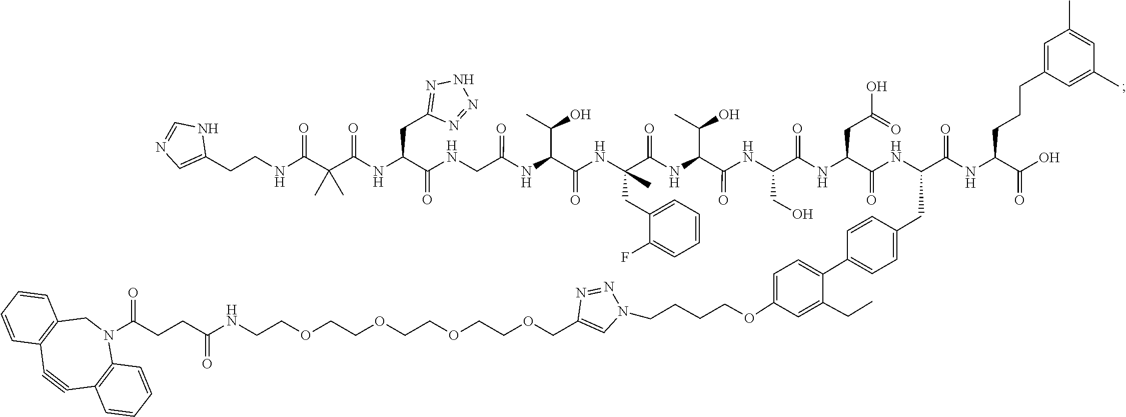 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP9 (SEQ ID NO: 93) | BCN- PEG4- triazole- P8 | 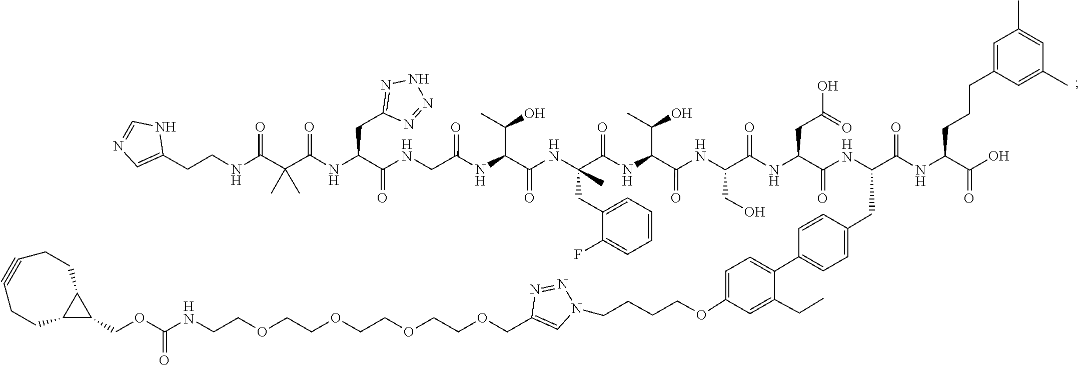 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP10 (SEQ ID NO: 94) | COT- PEG4- triazole- P8 | 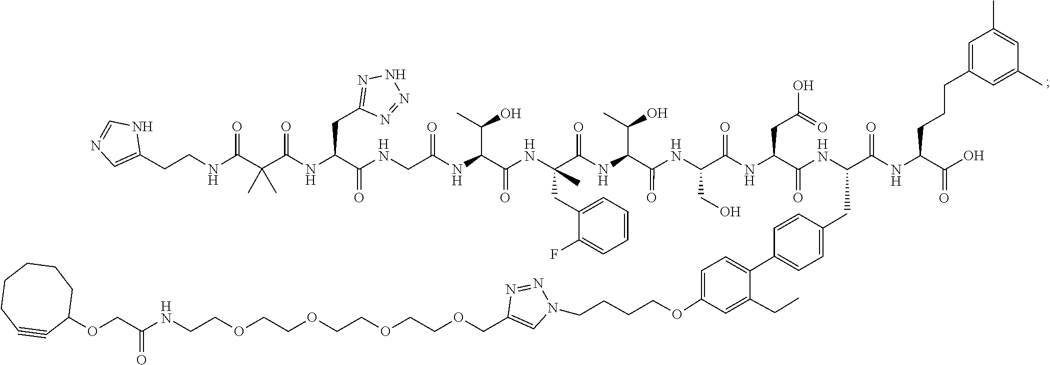 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP12 (SEQ ID NO: 95) | DIBAC- suc- PEG24- P11 | 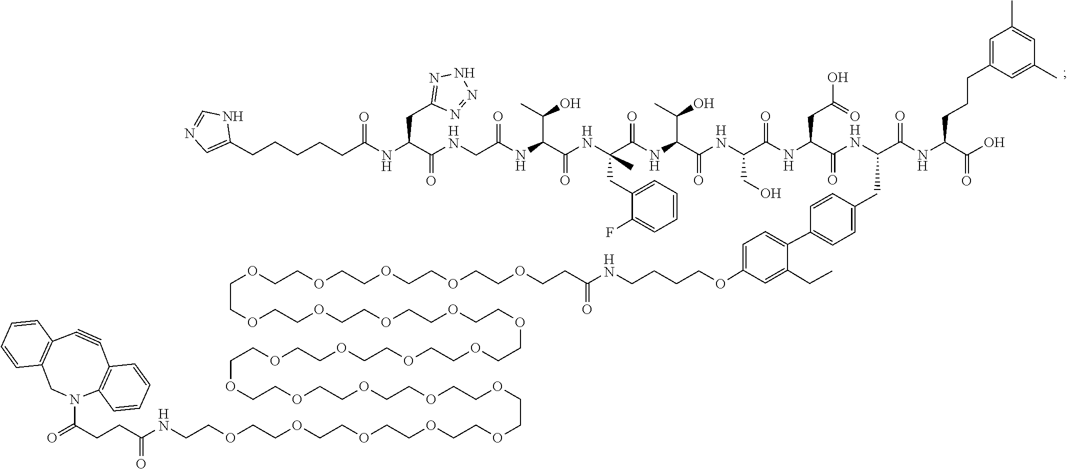 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)N[C@@H](CCCc2cc(C)cc(C)c2)C(=O)O)cc1] |
| LP13 (SEQ ID NO: 96) | DIBAC- suc- PEG24- P4 | 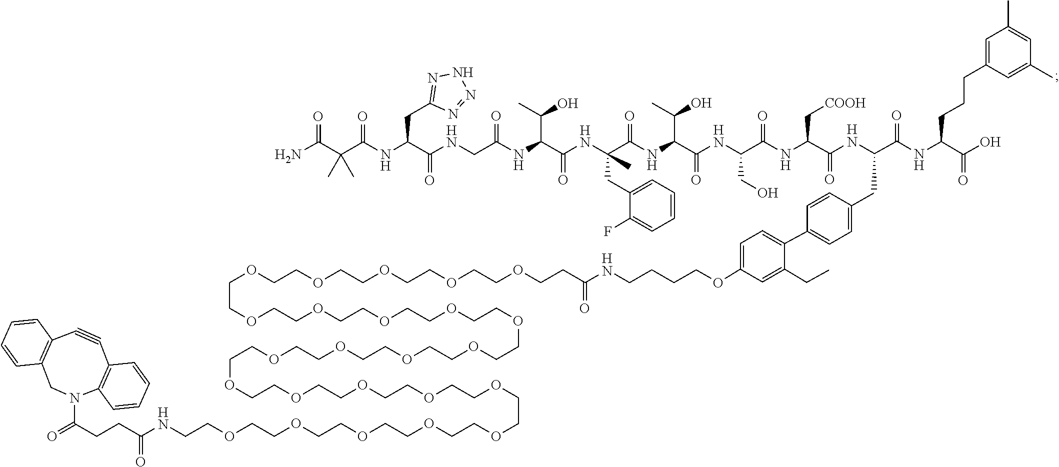 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(=O)O)cc1] |
| LP14 (SEQ ID NOS 97 and 98, respec- tively) | DIBAC- suc- G4S- P4 | 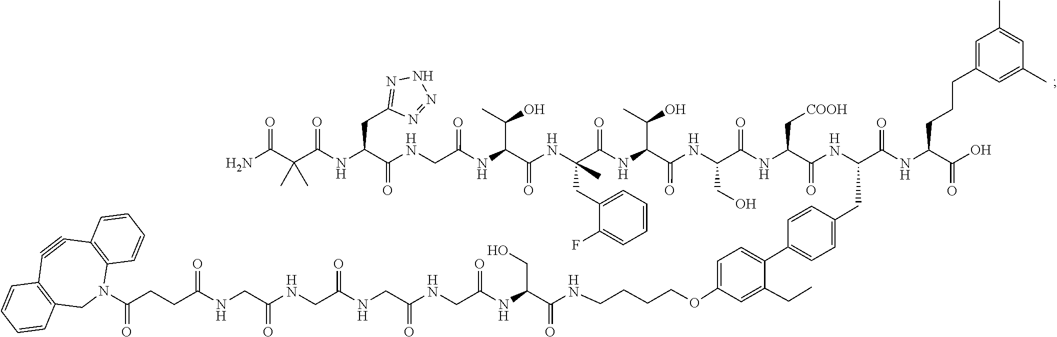 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)N[C@@H](CCCc2cc(C)cc(C)c2)C(=O)O)cc1] |
| LP17 (SEQ ID NOS 99 and 100, respec- tively) | DIBAC- suc- G₄S- G₄S- P9 | 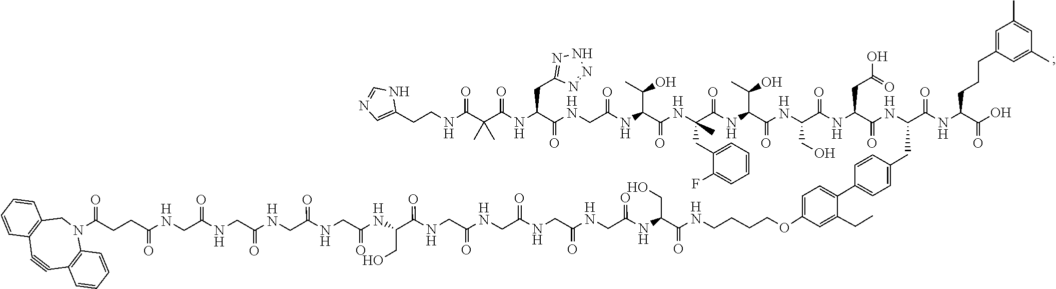 [SMILES: CCc1cc(OCCCCNC(=O)[C@H](CO)NC(=O)CNC(=O)CNC(=O)CNC(=O)CNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP18 (SEQ ID NOS 101 and 102, respec- tively) | BCN- NHC₂H₄CO- (glucose) SG₄- P9 | 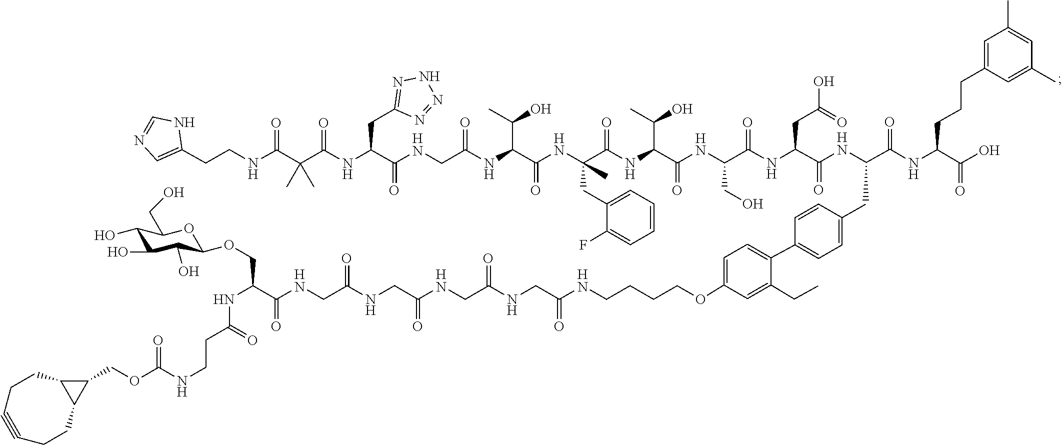 |
| LP19 (SEQ ID NOS 103 and 104, respec- tively) | COT- G₄- (R)Ser- P9 | 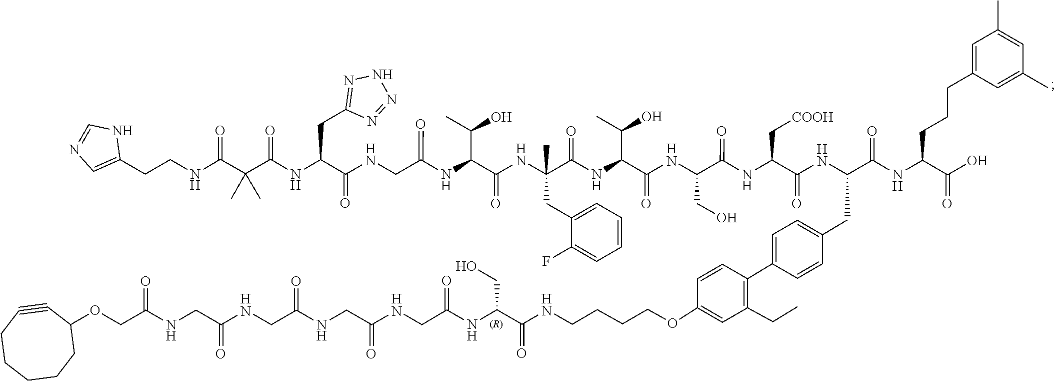 |
| LP20 (SEQ ID NOS 105 and 106, respec- tively) | DIBAC- suc- (glucose) SG₄- P9 | 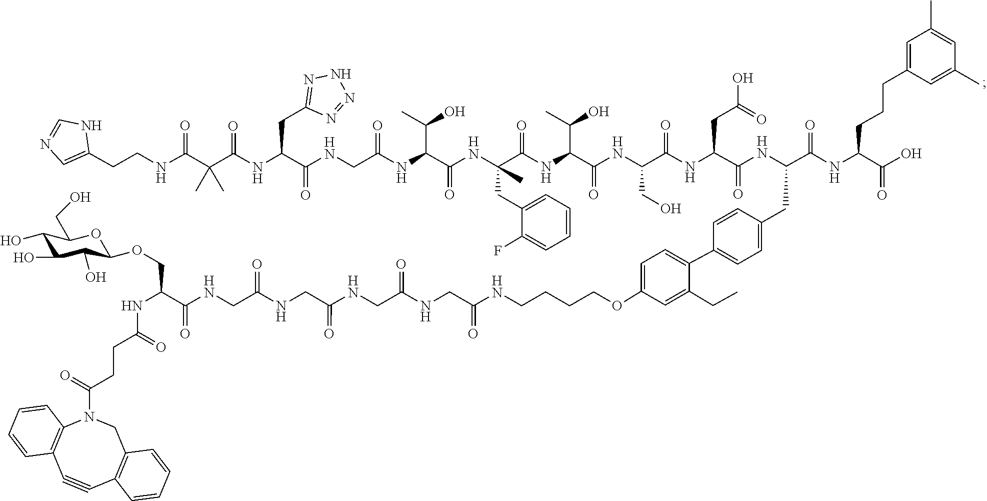 |
| LP21 (SEQ ID NO: 107) | DIBAC- suc- PEG24- P24 | 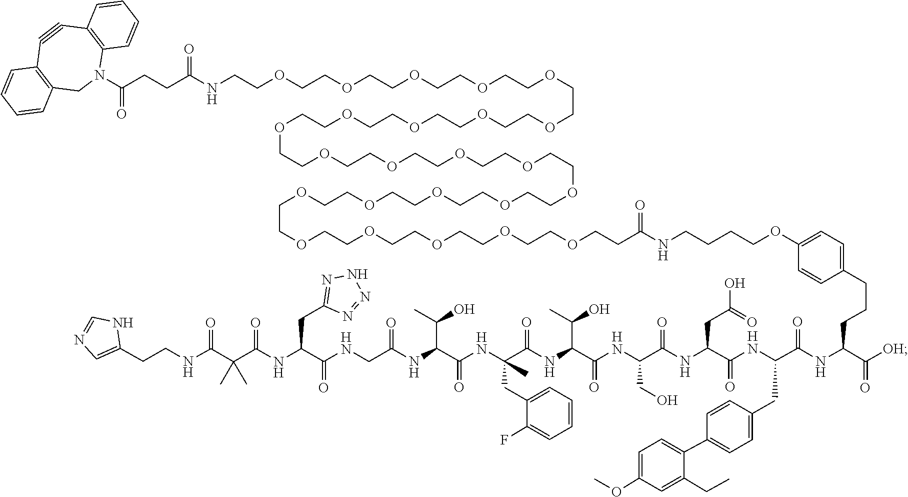 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4C#Cc4ccccc43)cc2)C(=O)O)cc1] |
| LP22 (SEQ ID NO: 108) | cyclo- dextrin- triazole- DIBAC- suc- PEG24- P9 | 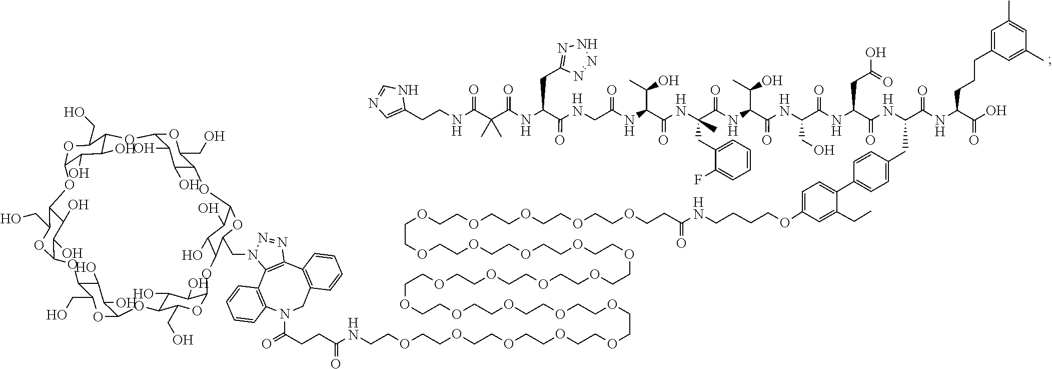 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP23 (SEQ ID NO: 109) | cyclo- dextrin- triazole- DIBAC- suc- PEG24- P24 | 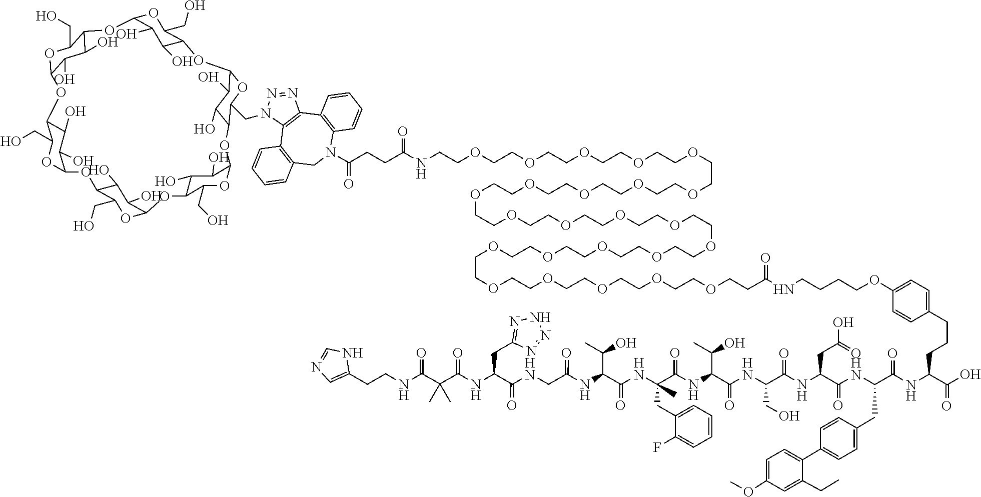 [SMILES: CCc1cc(OC)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2ccc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N3Cc4ccccc4-c4c(nnn4CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)-c4ccccc43)cc2)C(=O)O)cc1] |
| LP24 (SEQ ID NO: 110) | triazole- BCN- triazole- P8 | 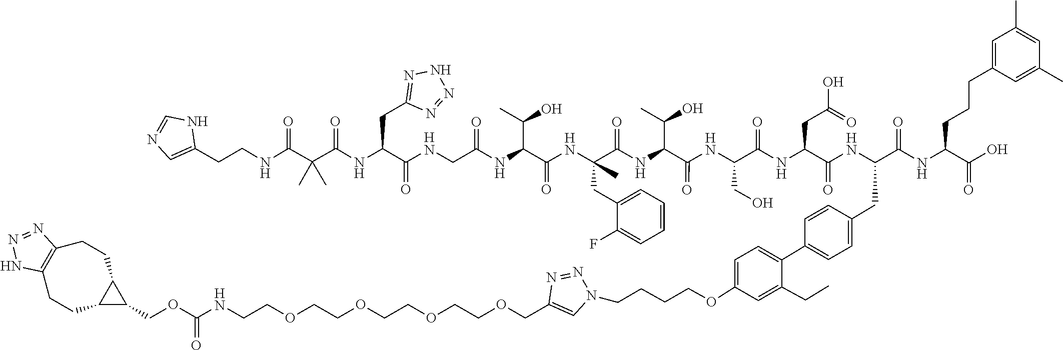 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP25 (SEQ ID NO: 111) | triazole- BCN- PEG4- carbamate- P9 | 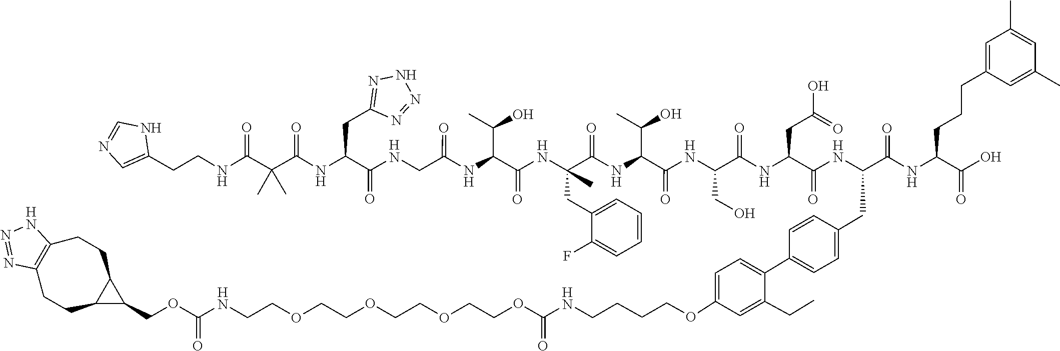 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP26 (SEQ ID NOS 112 and 113, respec- tively) | triazole- DIBAC- G4S- P9 | 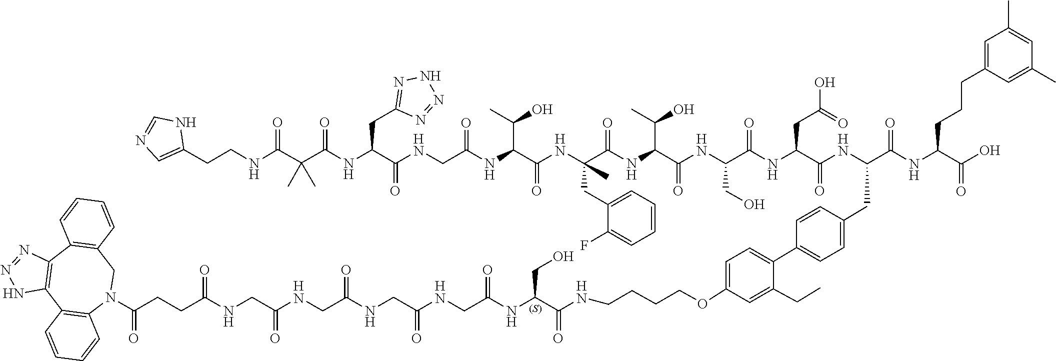 |
| LP27 (SEQ ID NO: 114) | DIBAC- suc- PEG8- triazole- P8 | 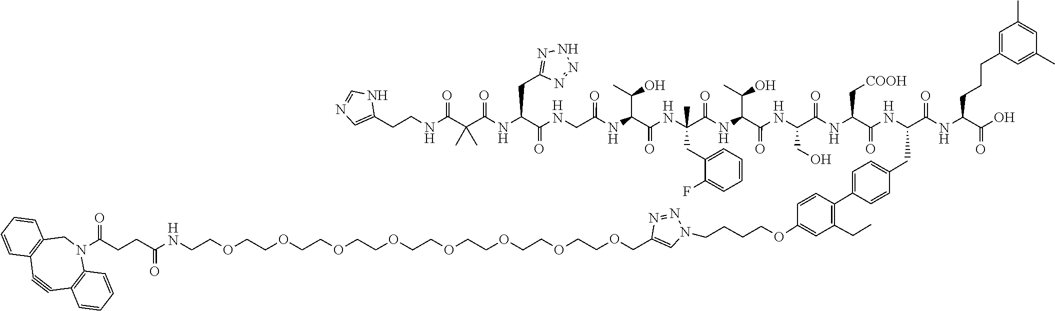 |
| LP28 (SEQ ID NO: 115) | triazole- DIBAC- suc- PEG8- triazole- P8 | 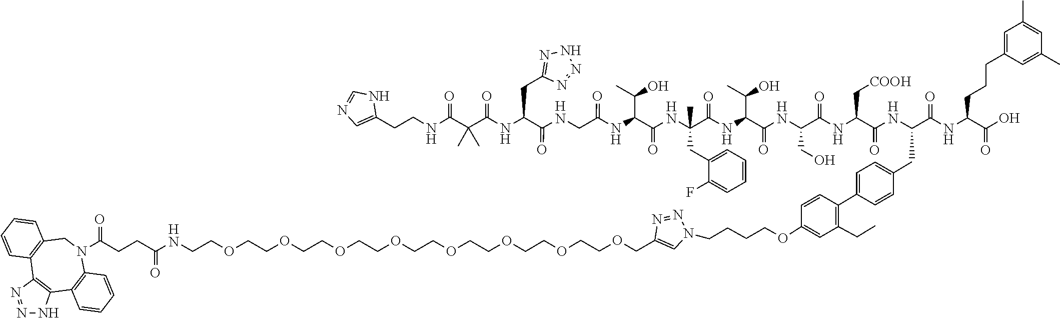 |
| LP29 (SEQ ID NO: 116) | NH2- PEG8- triazole- P19 | 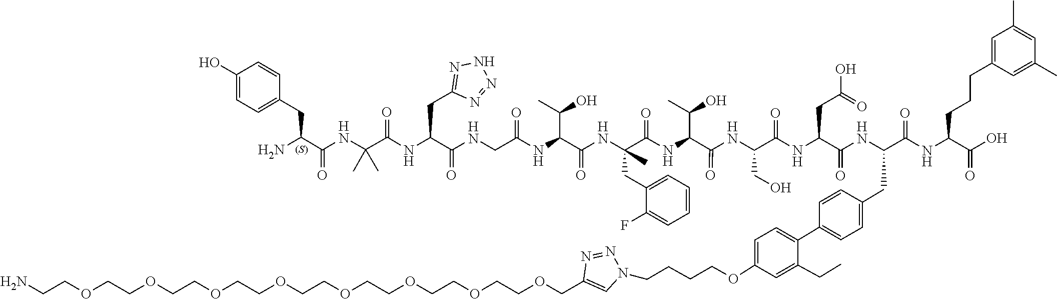 |
| LP30 (SEQ ID NO: 117) | NH2- PEG8- triazole- P35 | 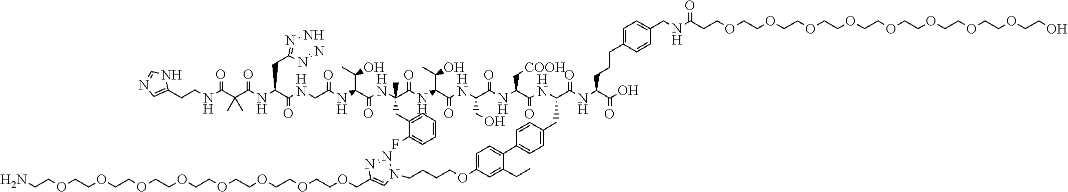 |
| LP31 (SEQ ID NO: 118) | NH2- PEG8- triazole- P8 | 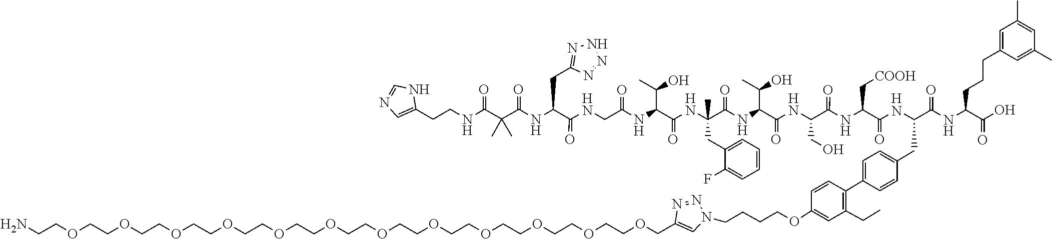 |
| LP32 (SEQ ID NO: 119) | NH2- PEG8- triazole- P8 acid | 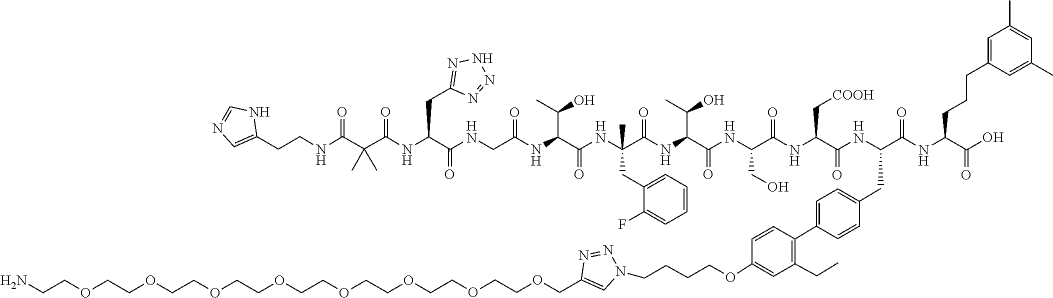 |
| LP33 (SEQ ID NO: 120) | E- PEG8- triazole- P8 | 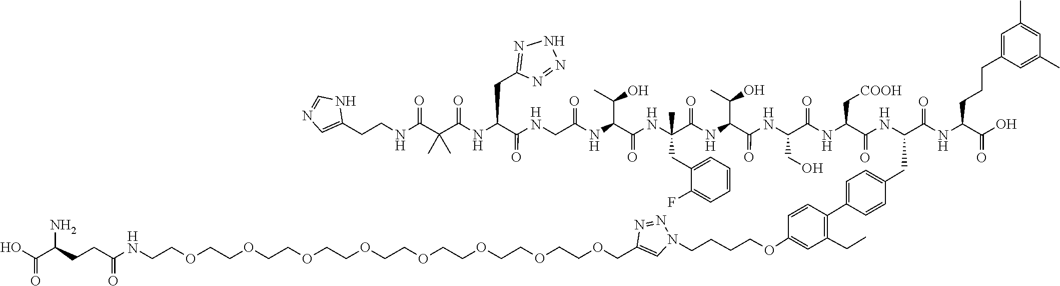 |
| LP34 (SEQ ID NOS 121 and 122, respec- tively) | GGTEPL- PEG8- triazole- P8 | 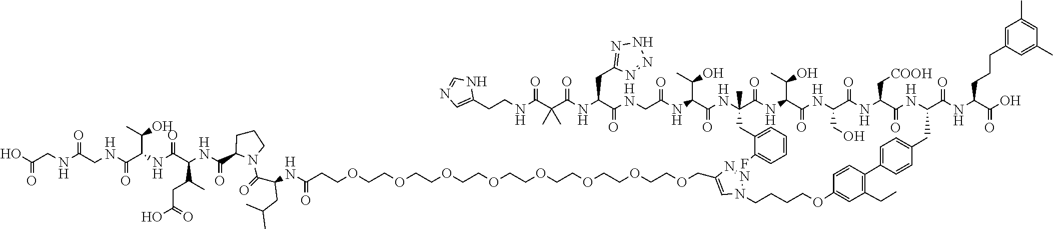 |
| LP35 (SEQ ID NOS 123 and 124, respec- tively) | Cbz- LLQGSG- PEG8- triazole- P8 | 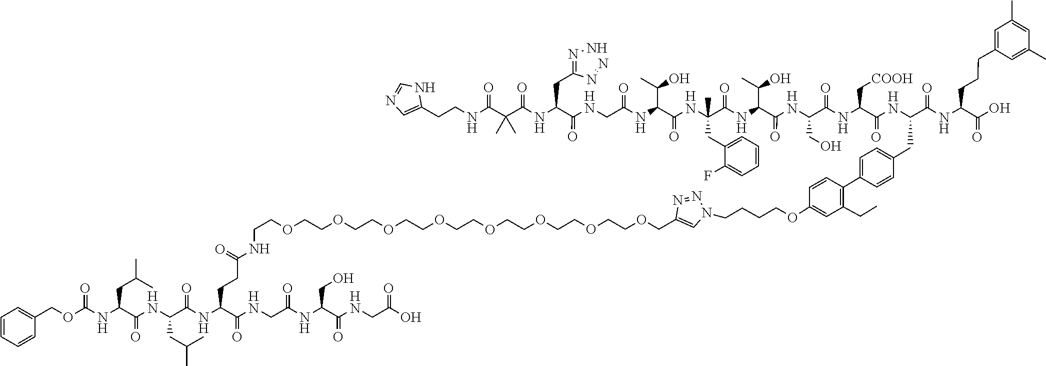 |
| LP36 (SEQ ID NOS 125 and 126, respec- tively) | G4S triazole- P40 | 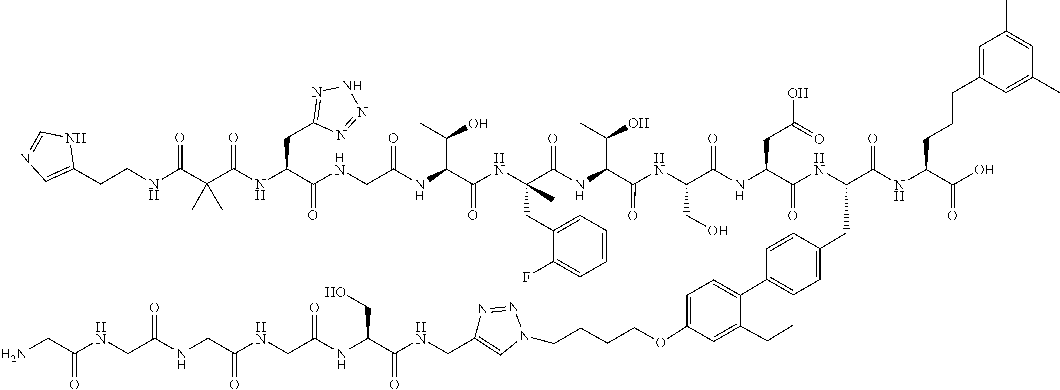 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP37 (SEQ ID NOS 127 and 128, respec- tively) | SG4- triazole- P40 | 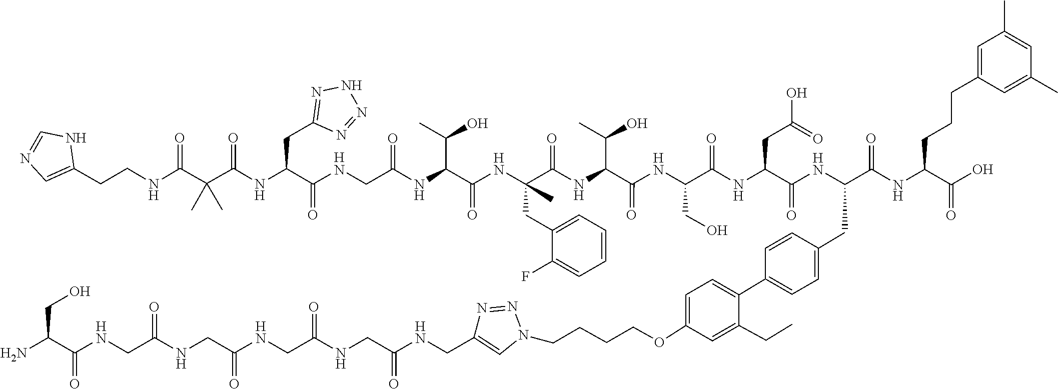 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP38 (SEQ ID NOS 129 and 130, respec- tively) | G2SG2SG2 triazole- P40 | 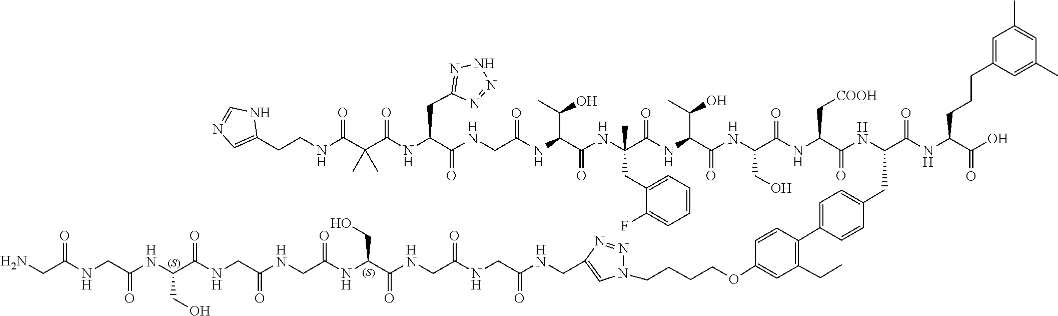 |
| LP39 (SEQ ID NOS 131 and 132, respec- tively) | G4SG4 triazole- P40 | 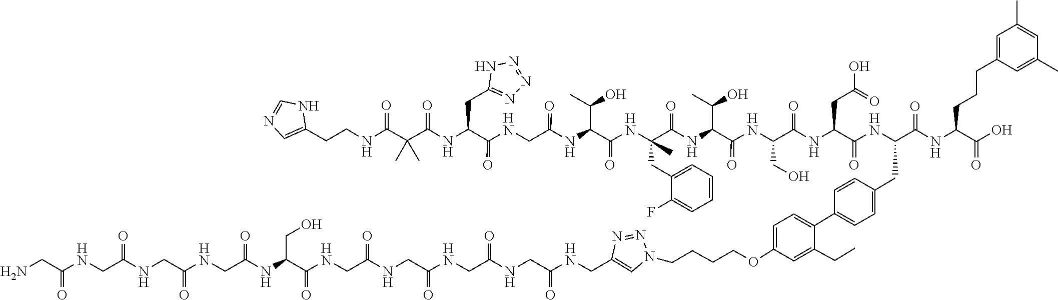 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP40 (SEQ ID NOS 133 and 134, respec- tively) | G4SG4SG4 triazole- P40 | 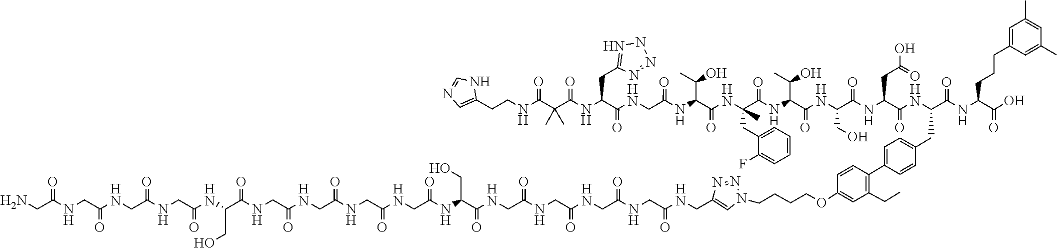 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)N[C@@H](CCCc2cc(C)cc(C)c2)C(=O)O)cc1] |
| LP41 (SEQ ID NOS 135 and 136, respec- tively) | G2SG2S- G2SG2 triazole- P40 | 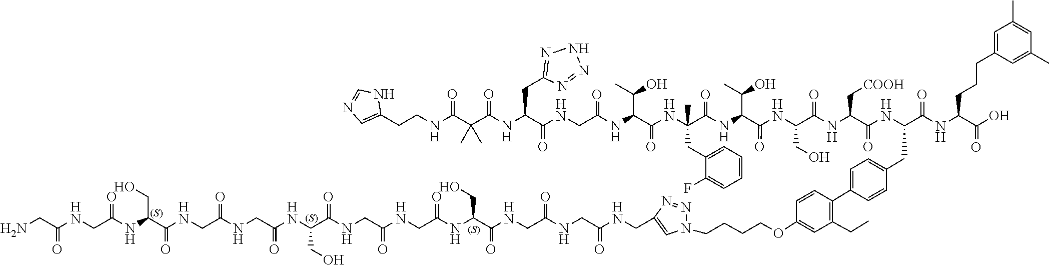 |
| LP42 (SEQ ID NOS 137 and 138, respec- tively) | C18- diacid- Glu- (AEEA)2- G4SG4- triazole- P40 | 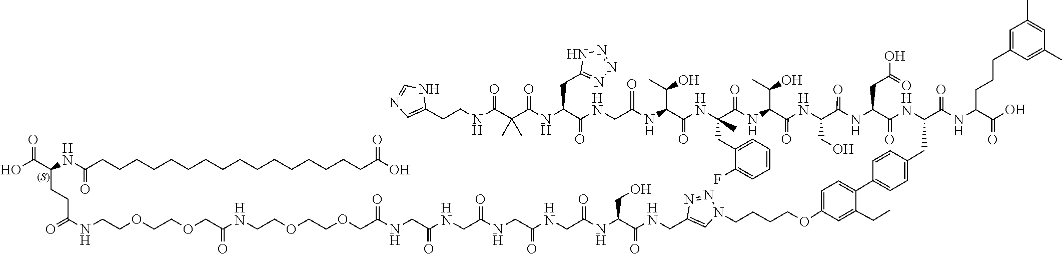 |
| LP43 (SEQ ID NOS 139 and 140, respec- tively) | C18- diacid- Glu- (AEEA)2- G4SG4- triazole- P40 | 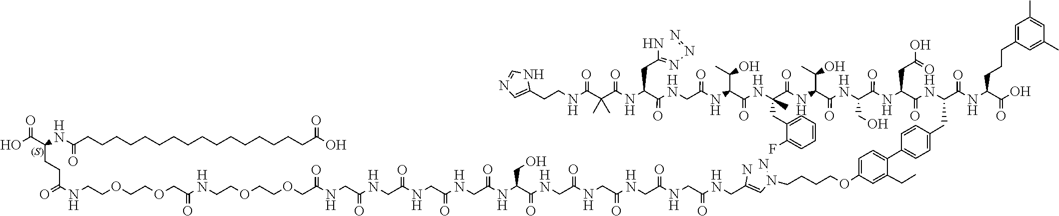 |
| LP44 (SEQ ID NO: 141) | C18- diacid- Glu- (AEEA)2- NH2- PEG12- P40 | 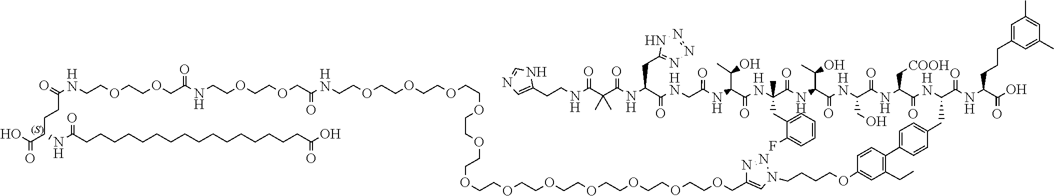 |
| LP45 (SEQ ID NO: 142) | C18- diacid- Glu- (AEEA)2- NH2- PEG8- P35 | 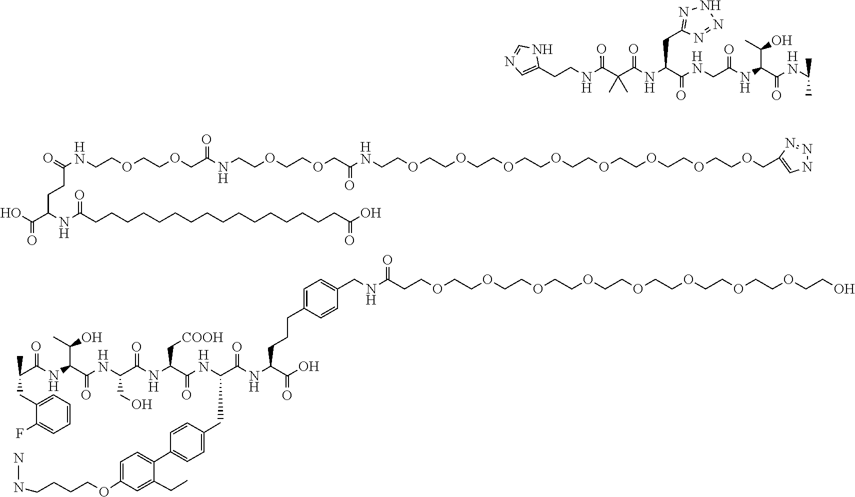 |
| LP46 (SEQ ID NO: 143) | Leu- Leu- Glu- PEG8- P40 | 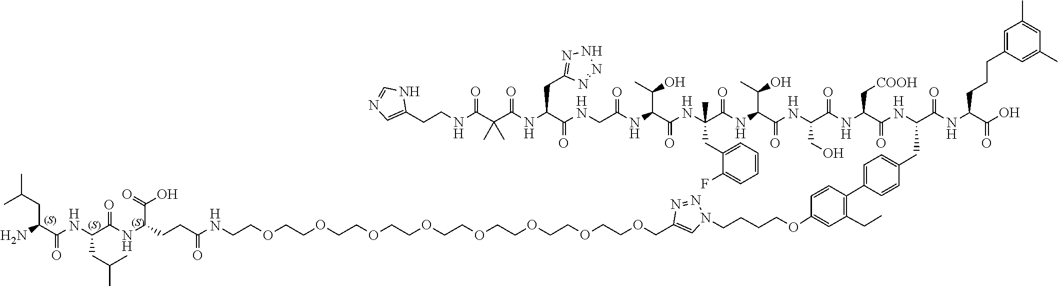 |
| LP47 (SEQ ID NO: 144) | M6447 series | 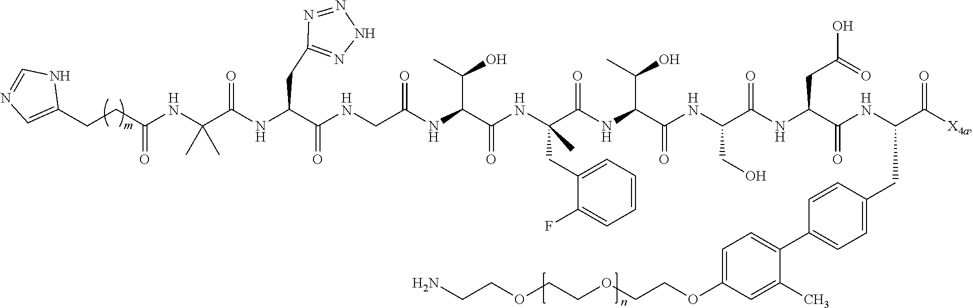 |
| | | X₄ₐ is —OH or —NH₂; m is an integer of one to seven; n is an integer of one to twelve, |
| LP48 (SEQ ID NO: 145) | M6447 series | 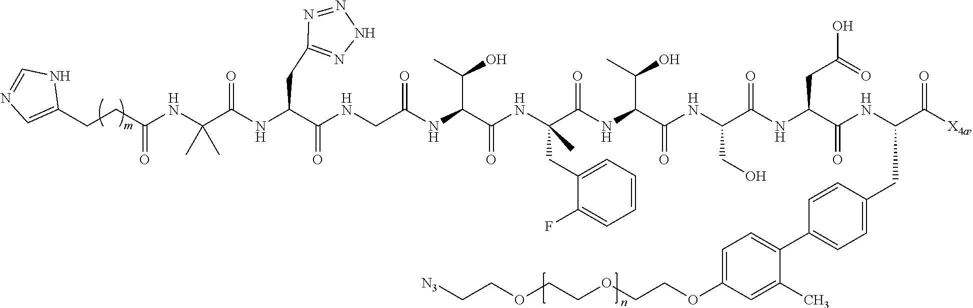 |
| | | X₄ₐ is —OH or —NH₂; m is an integer of one to seven; n is an integer of one to twelve, |

or a pharmaceutically acceptable salt thereof.

In another embodiment, the linker-payloads as described above have has the structure selected from the group consisting of:

| LP′# | Structure |
|---|---|
| LP1′ (SEQ ID NO: 206) | 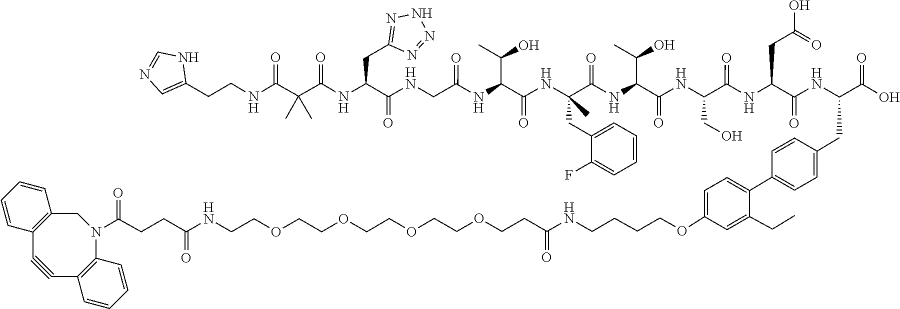 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP2′ (SEQ ID NO: 207) | 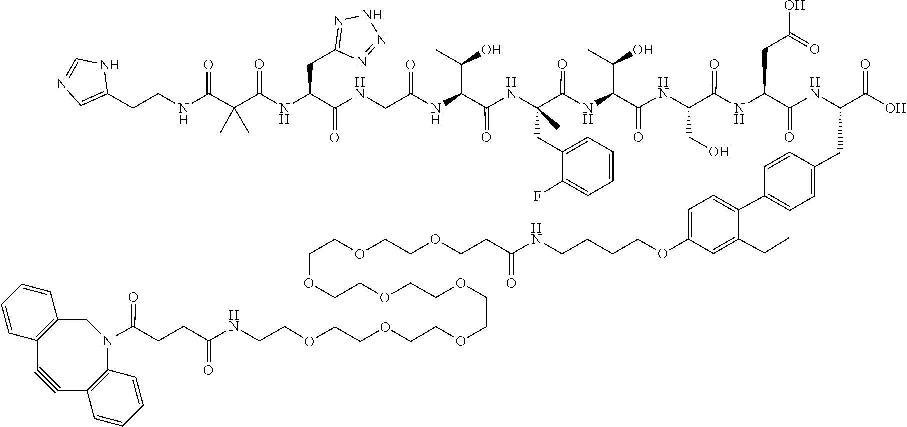 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP3′ (SEQ ID NO: 208) | 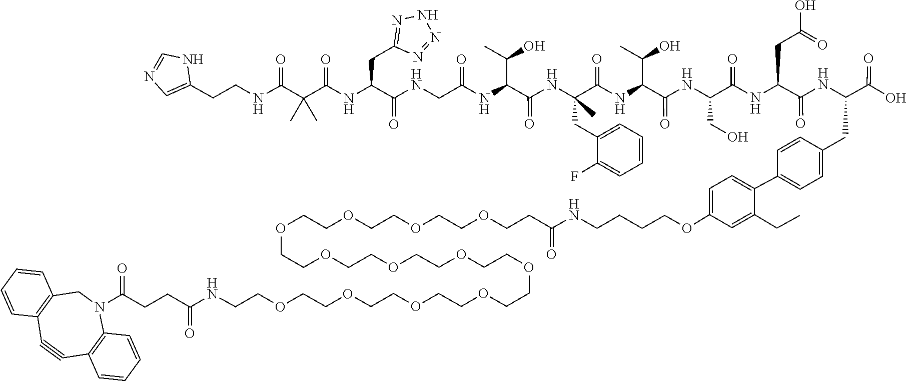 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP4′ (SEQ ID NO: 209) | 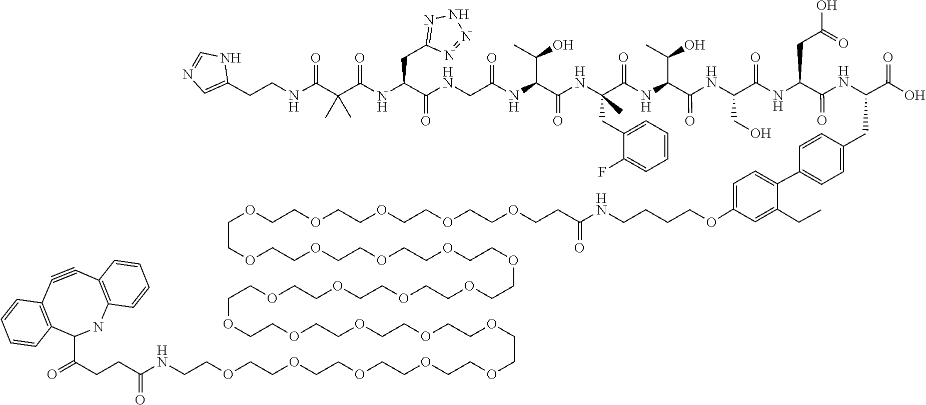 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)C2Nc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP5′ (SEQ ID NO: 210) | 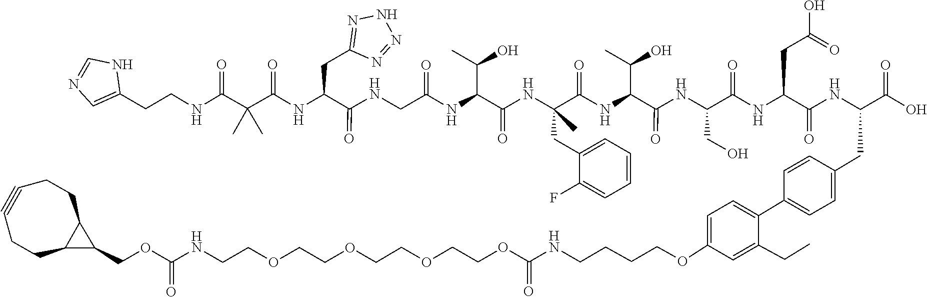 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP6′ (SEQ ID NO: 211 and 212 respectively) | 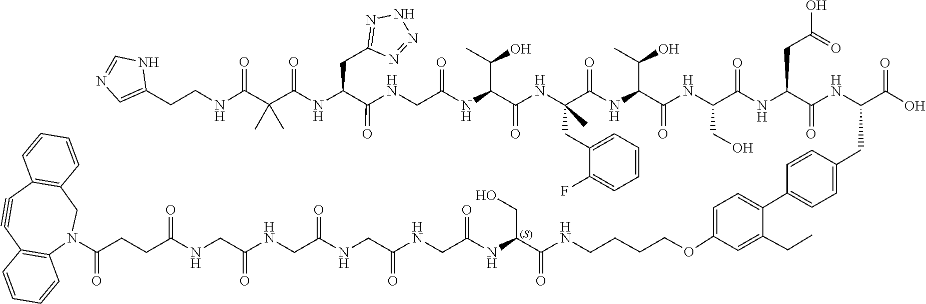 |
| LP7′ (SEQ ID NO: 213 and 214 respectively) | 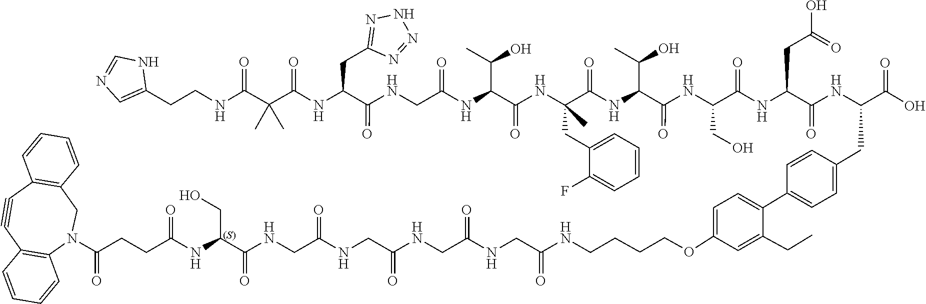 |
| LP8′ (SEQ ID NO: 215) | 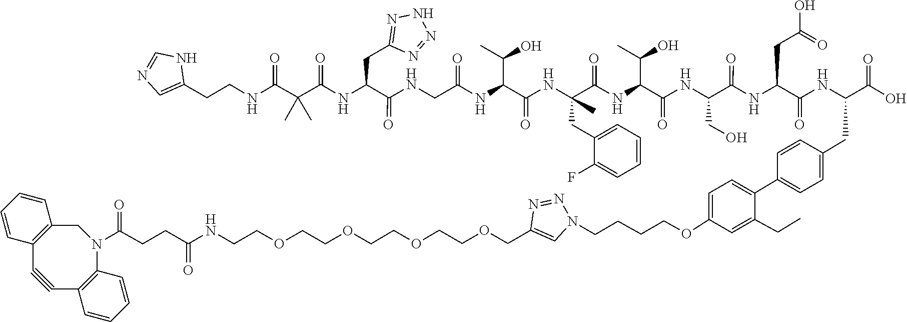 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP9′ (SEQ ID NO: 216) | 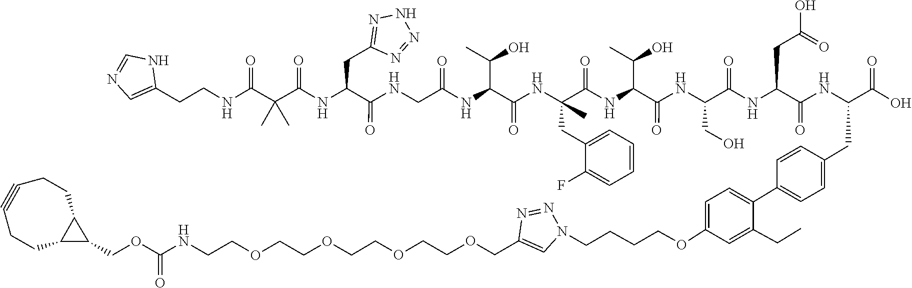 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP10′ (SEQ ID NO: 217) | 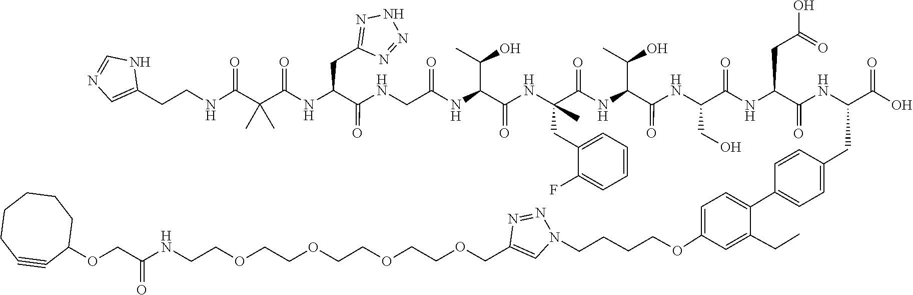 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP11′ (SEQ ID NO: 218) | 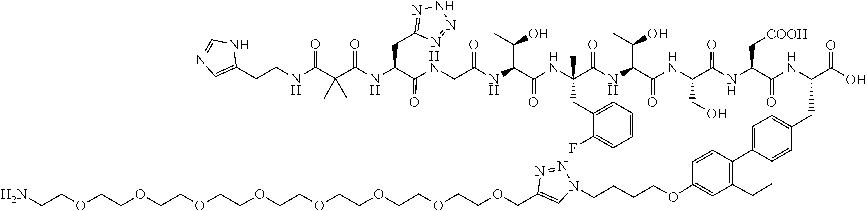 |
| LP12′ (SEQ ID NO: 219) | 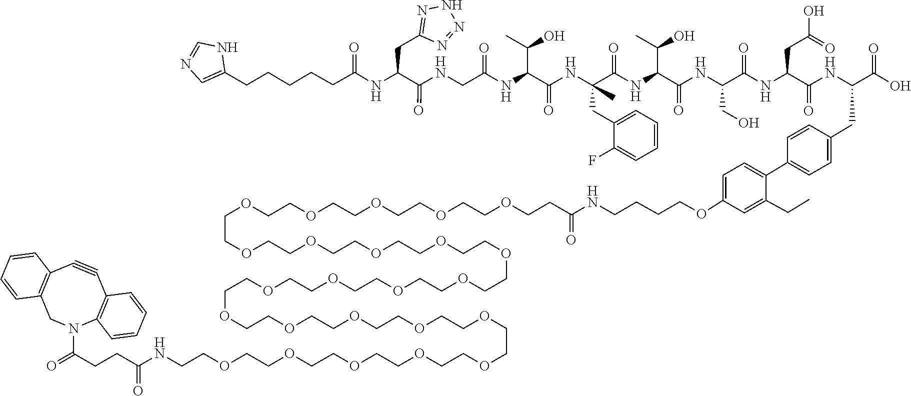 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)O)cc1] |
| LP13′ (SEQ ID NO: 220) | 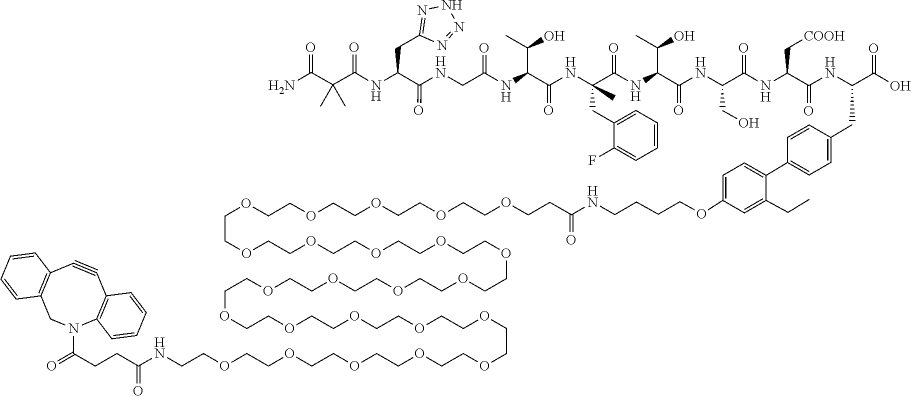 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)O)cc1] |
| LP14′ (SEQ ID NO: 221 and 222 respectively) | 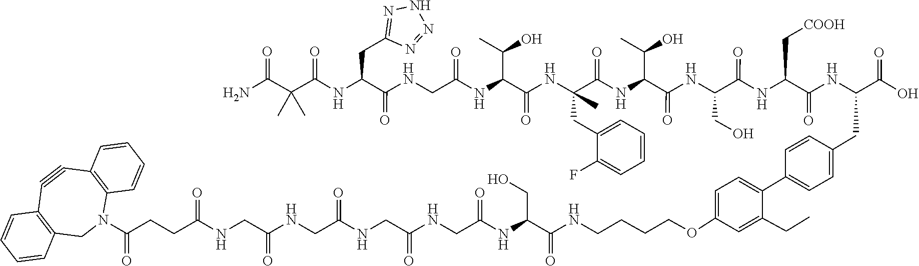 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)O)cc1] |
| LP17′ (SEQ ID NO: 223 and 224 respectively) | 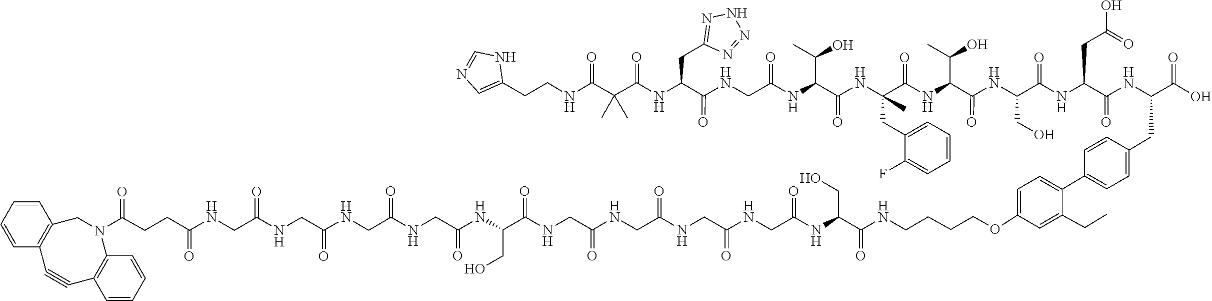 [SMILES: CCc1cc(OCCCCNC(=O)[C@H](CO)NC(=O)CNC(=O)CNC(=O)CNC(=O)CNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP18′ (SEQ ID NO: 225 and 226 respectively) | 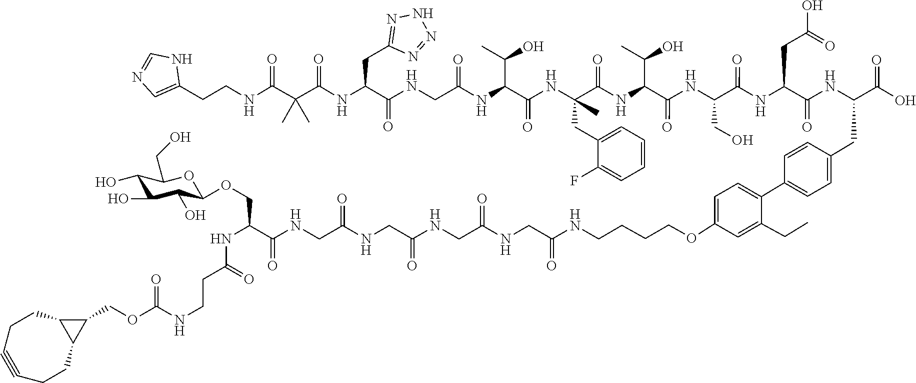 |
| LP19′ (SEQ ID NO: 227 and 228 respectively) | 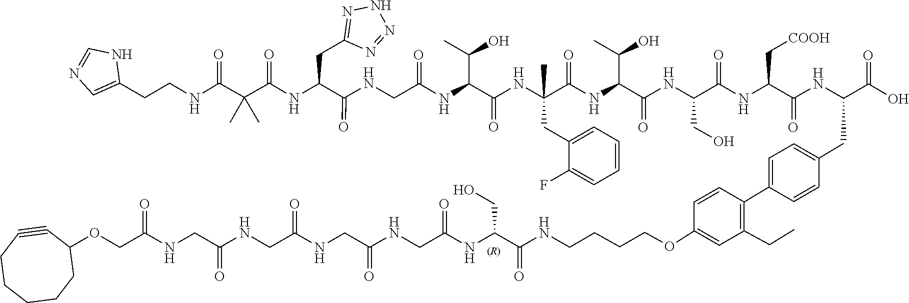 |
| LP20′ (SEQ ID NO: 229 and 230 respectively) | 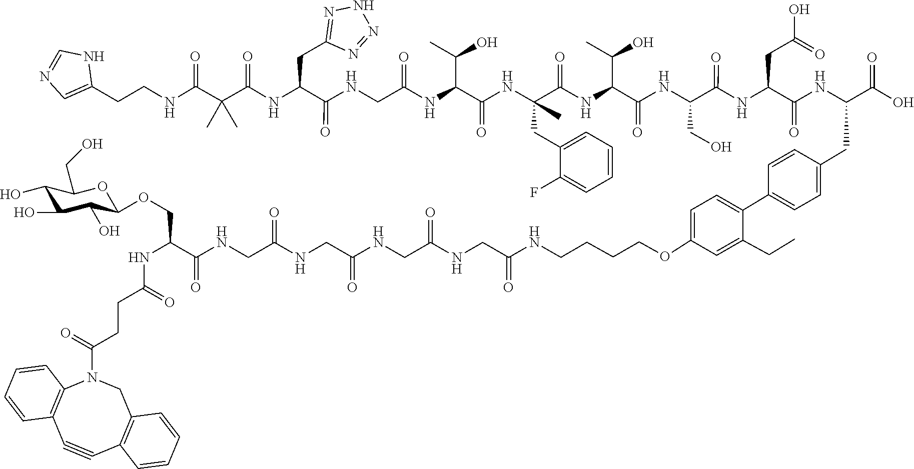 |
| LP22′ (SEQ ID NO: 231) | 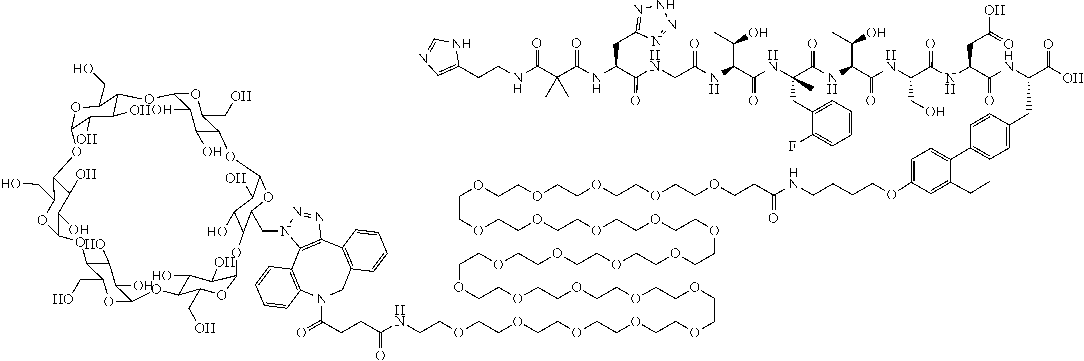 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP24′ (SEQ ID NO: 232) | 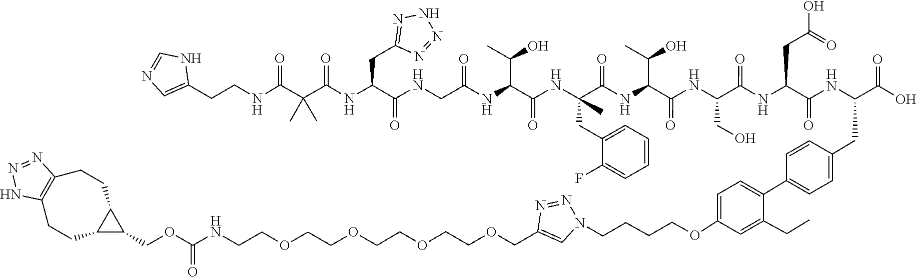 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP25′ (SEQ ID NO: 233) | 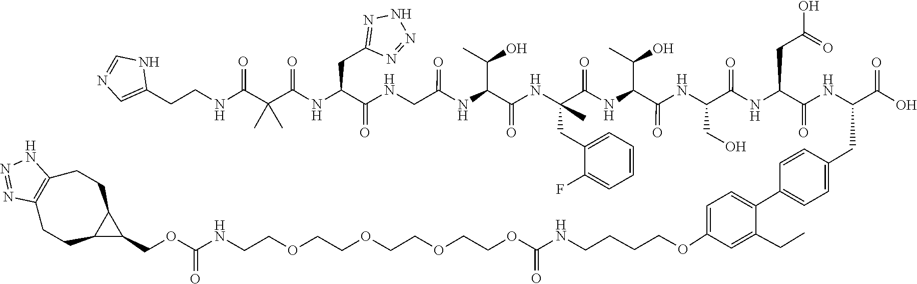 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP26′ (SEQ ID NO: 234 and 235 respectively) | 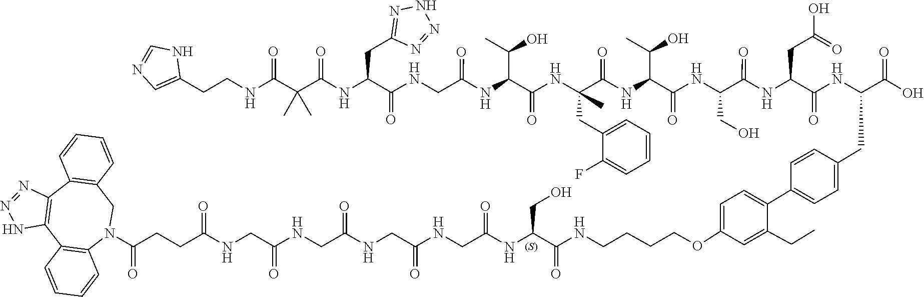 |
| LP27′ (SEQ ID NO: 236) | 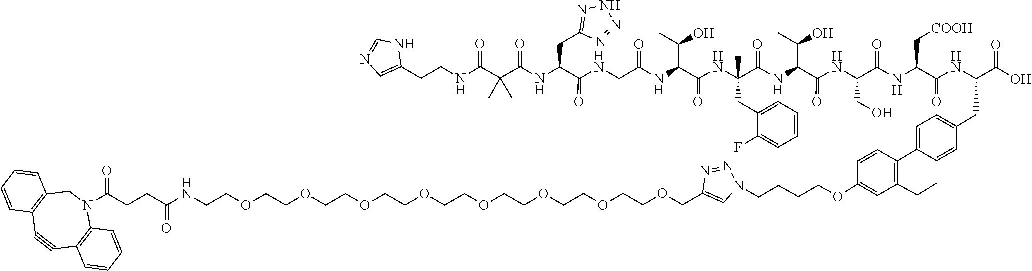 |
| LP28′ (SEQ ID NO: 237) | 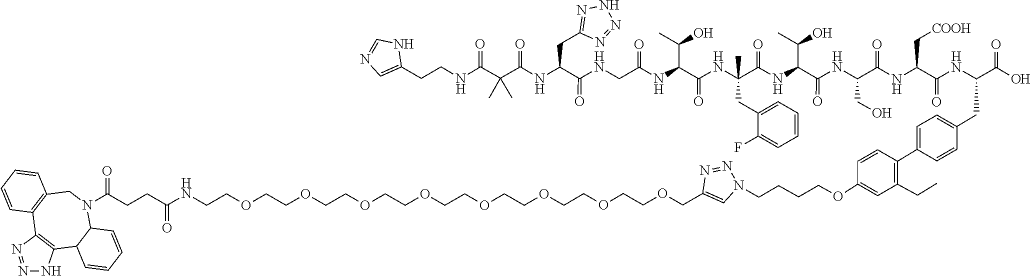 |
| LP29′ (SEQ ID NO: 238) | 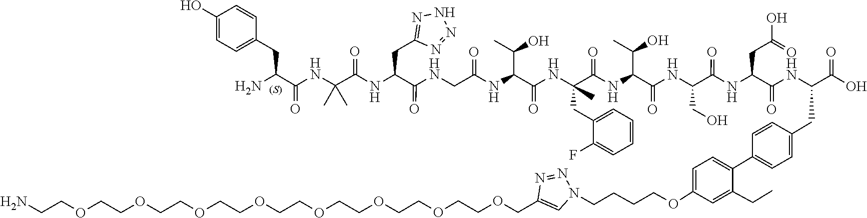 |
| LP30′ (SEQ ID NO: 239) | 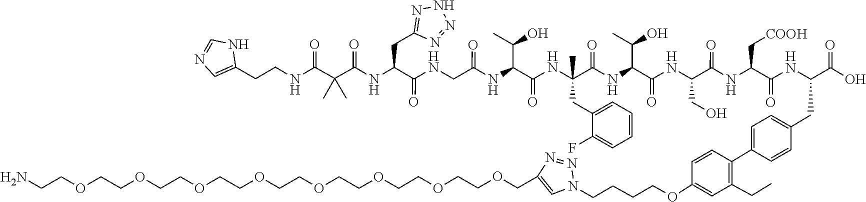 |
| LP31′ (SEQ ID NO: 240) | 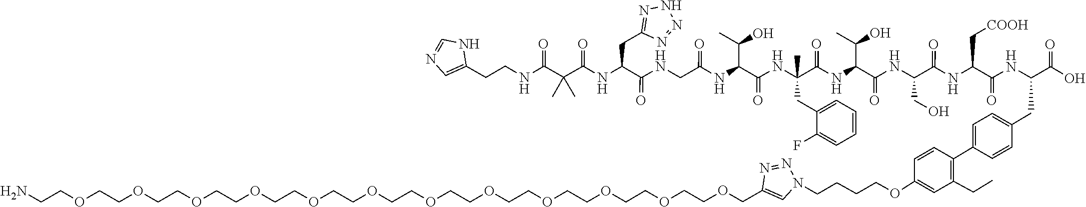 |
| LP33′ (SEQ ID NO: 241) | 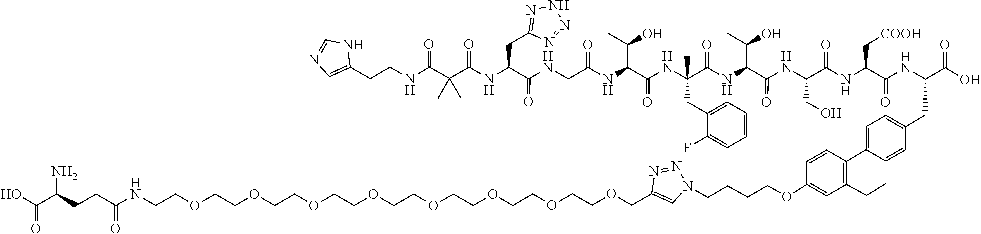 |
| LP34′ (SEQ ID NO: 242 and 243 respectively) | 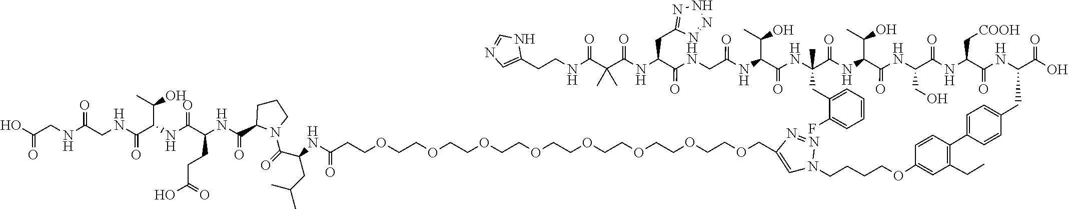 |
| LP35′ (SEQ ID NO: 244 and 245 respectively) | 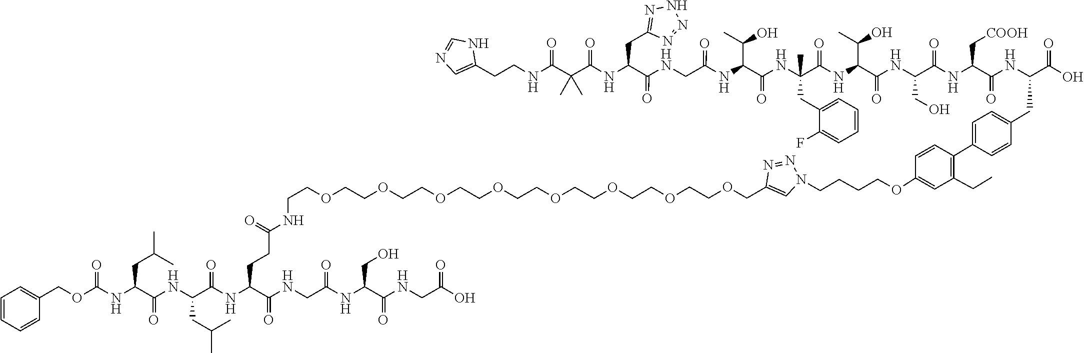 |
| LP36′ (SEQ ID NO: 246 and 352 respectively) | 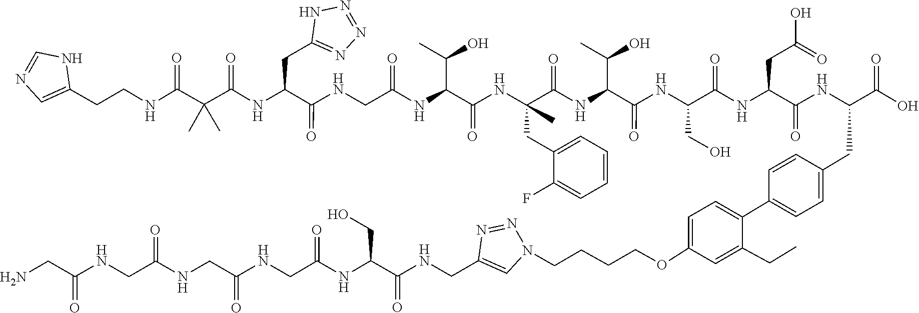 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP37′ (SEQ ID NO: 247 and 248 respectively) | 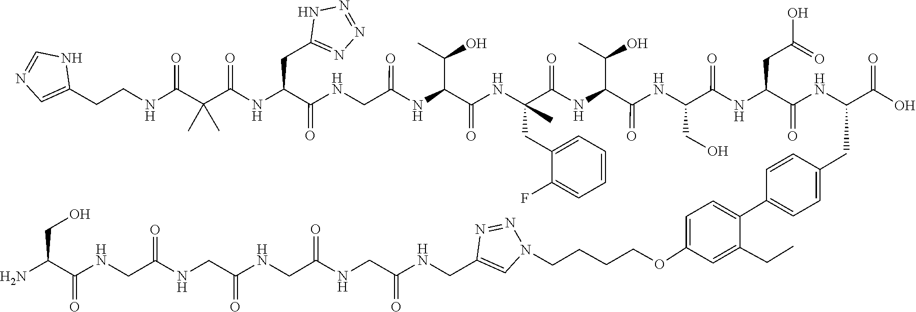 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP38′ (SEQ ID NO: 249 and 67 respectively) | 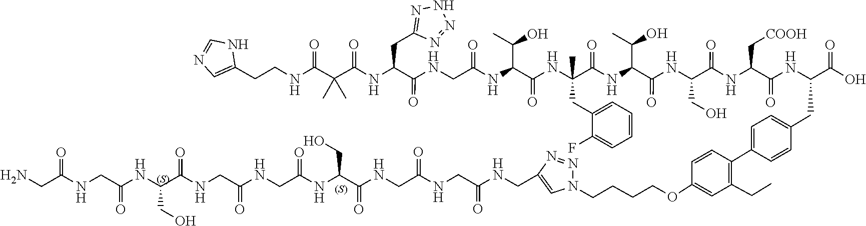 |
| LP39′ (SEQ ID NO: 250 and 251 respectively) | 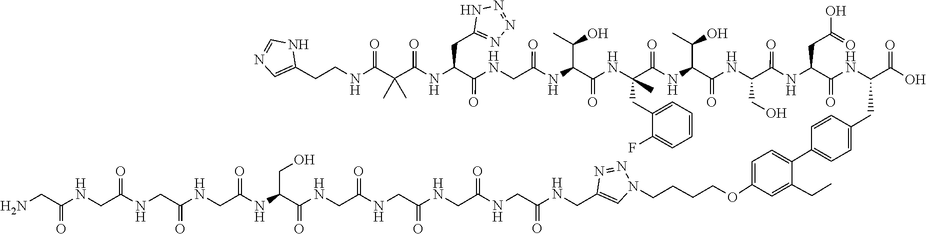 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP40′ (SEQ ID NO: 252 and 253 respectively) | 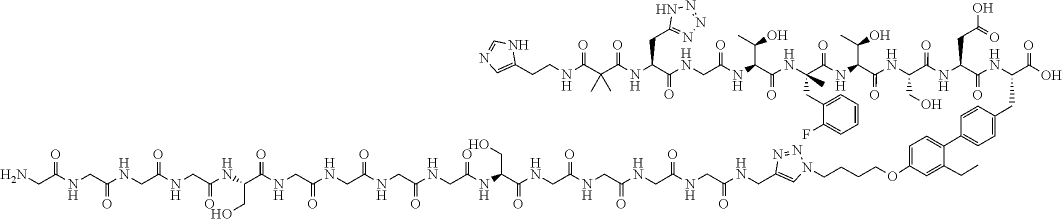 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP41′ (SEQ ID NO: 254 and 255 respectively) | 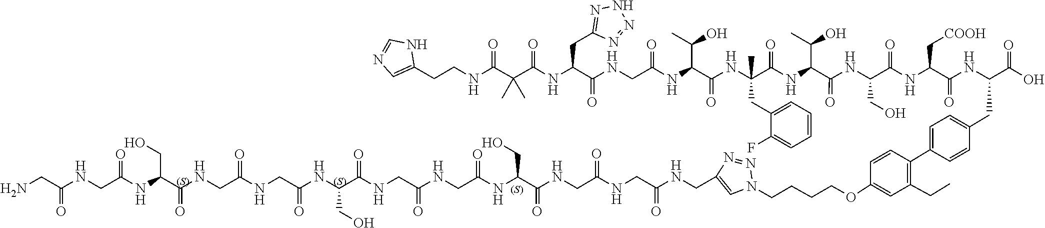 |
| LP42′ (SEQ ID NO: 256 and 257 respectively) | 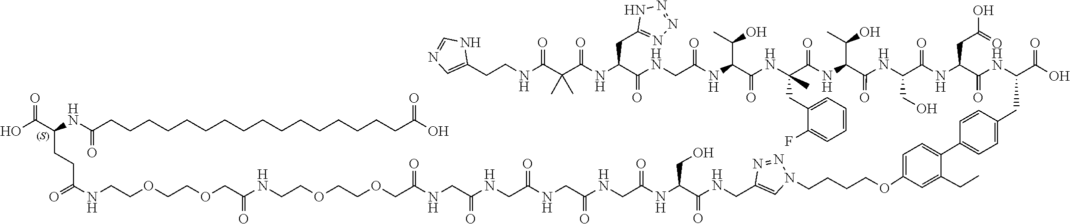 |
| LP43′ (SEQ ID NO: 258 and 259 respectively) | 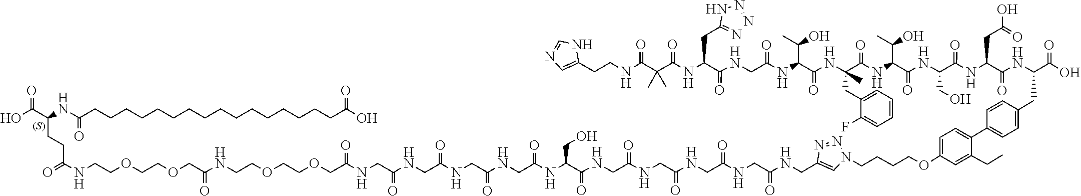 |
| LP44′ (SEQ ID NO: 260) | 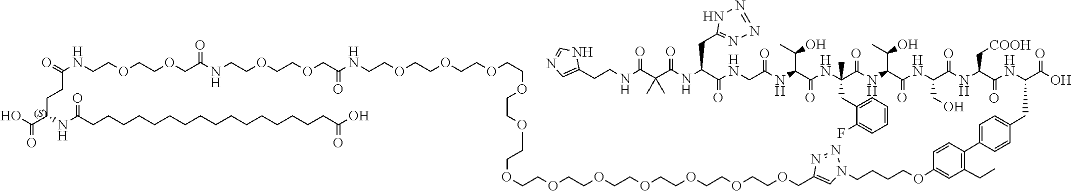 |
| LP45′ (SEQ ID NO: 261) | 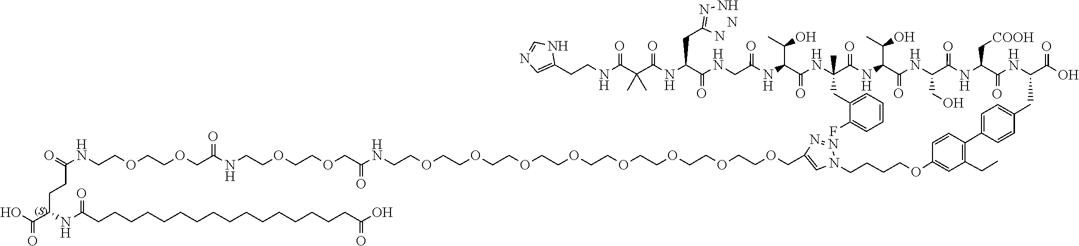 |
| LP46′ (SEQ ID NO: 262) | 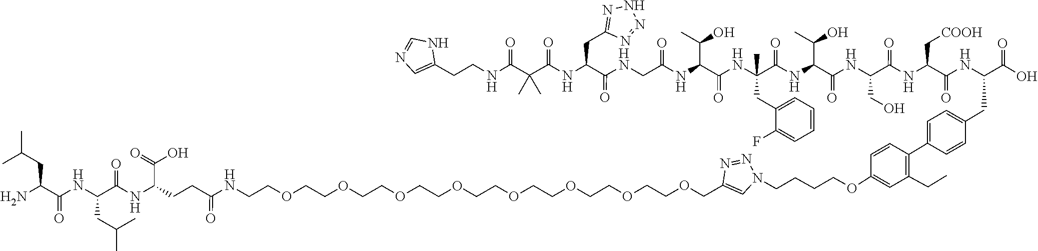 |
| LP1′a (SEQ ID NO: 263) | 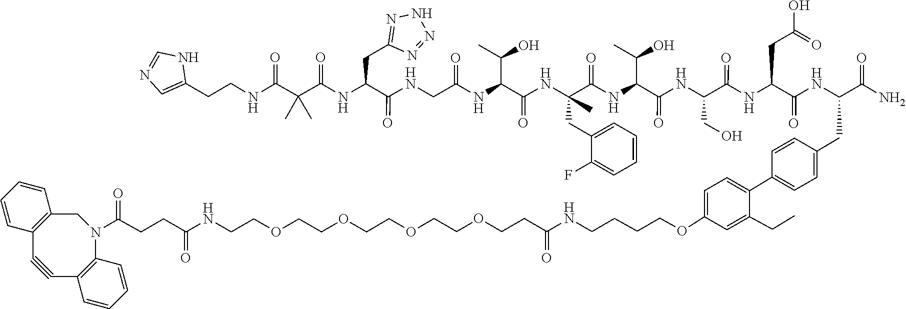 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP2′a (SEQ ID NO: 264) | 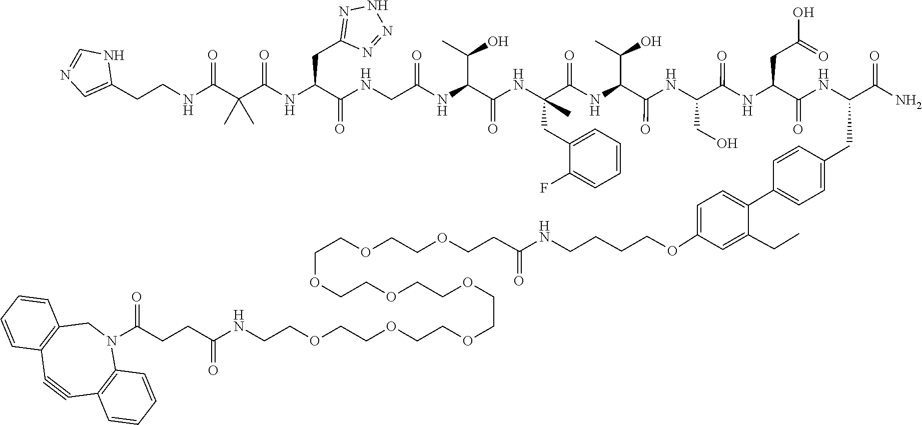 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP3′a (SEQ ID NO: 265) | 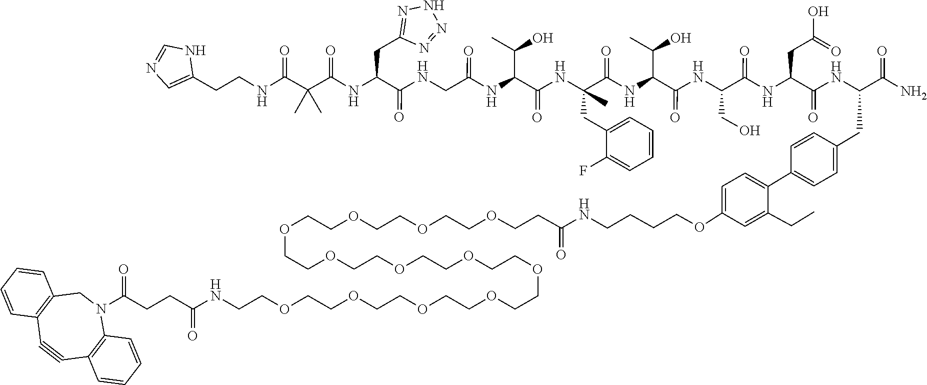 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP4′a (SEQ ID NO: 266) | 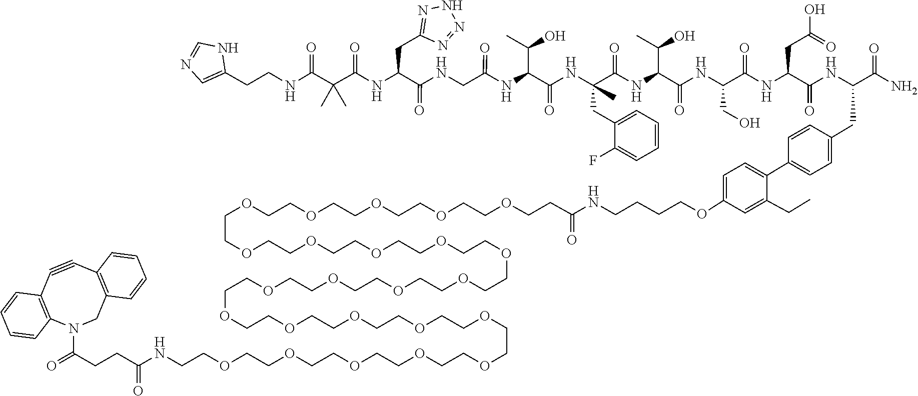 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP5′a (SEQ ID NO: 267) | 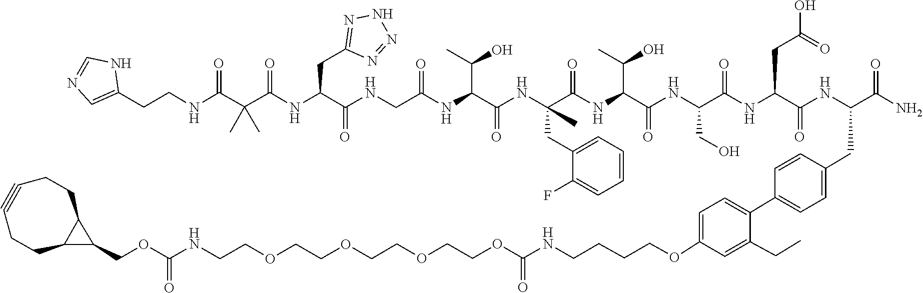 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP6′a (SEQ ID NO: 268 and 269 respectively) | 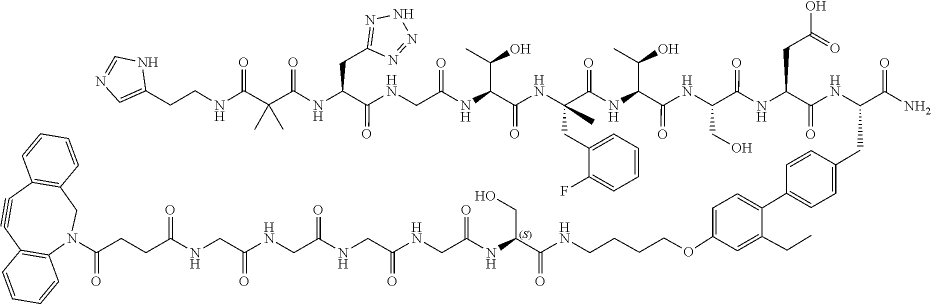 |
| LP7′a (SEQ ID NO: 270 and 271 respectively) | 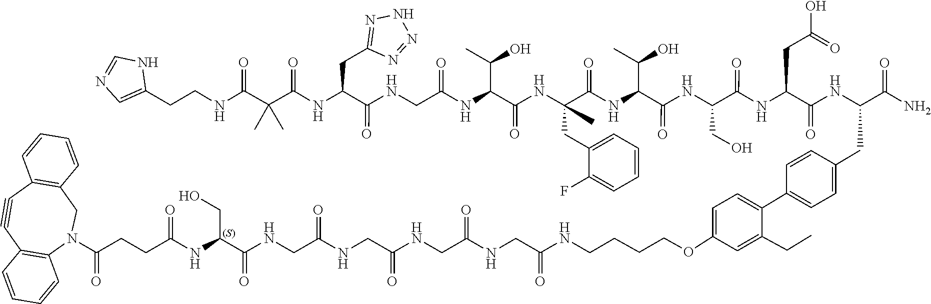 |
| LP8′a (SEQ ID NO: 272) | 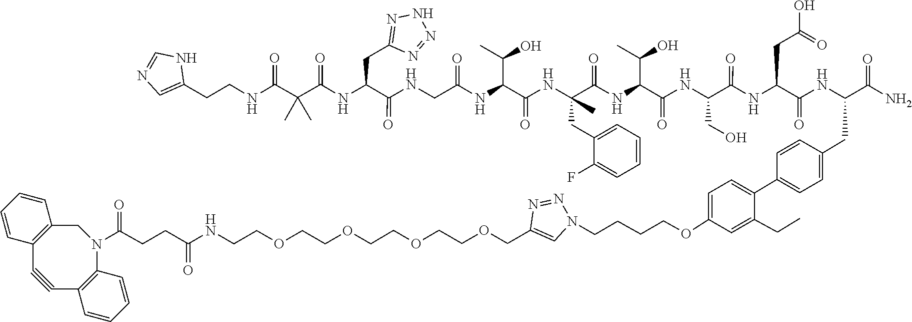 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP9′a (SEQ ID NO: 273) | 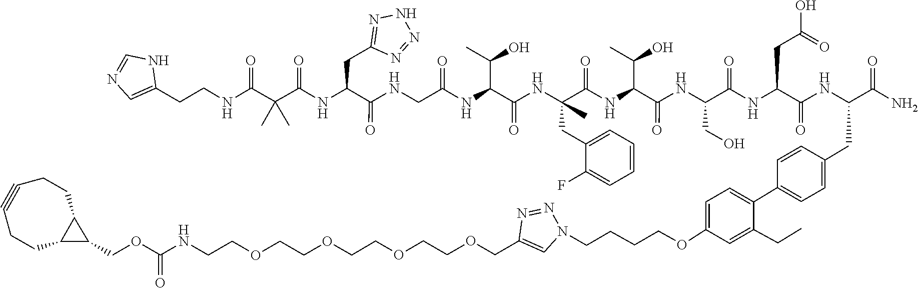 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP10′a (SEQ ID NO: 274) | 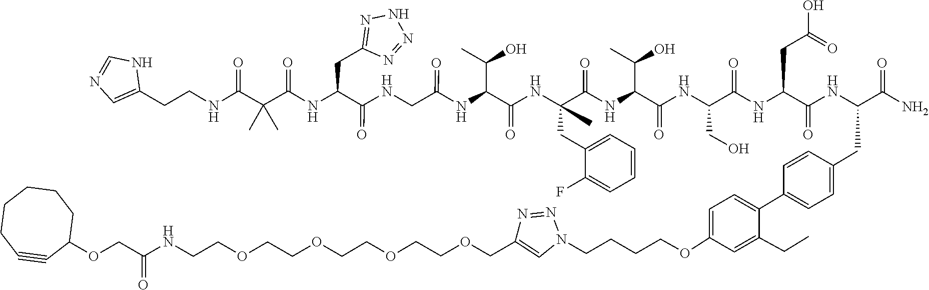 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP11′a (SEQ ID NO: 275) | 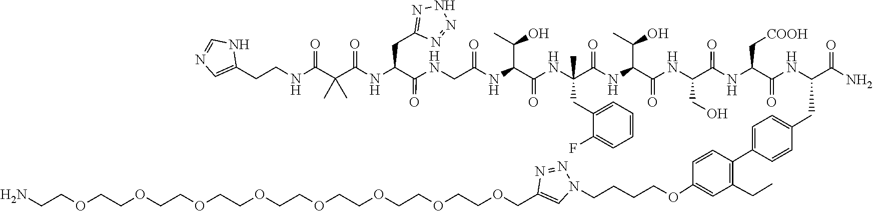 |
| LP12′a (SEQ ID NO: 276) | 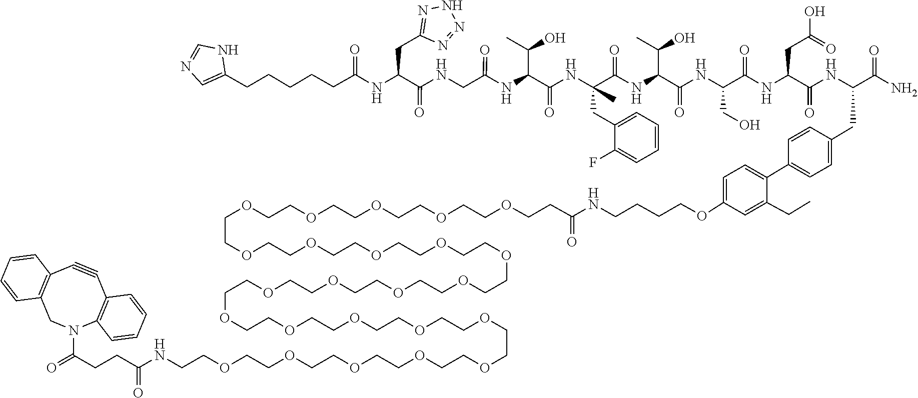 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(N)=O)cc1] |
| LP13′a (SEQ ID NO: 277) | 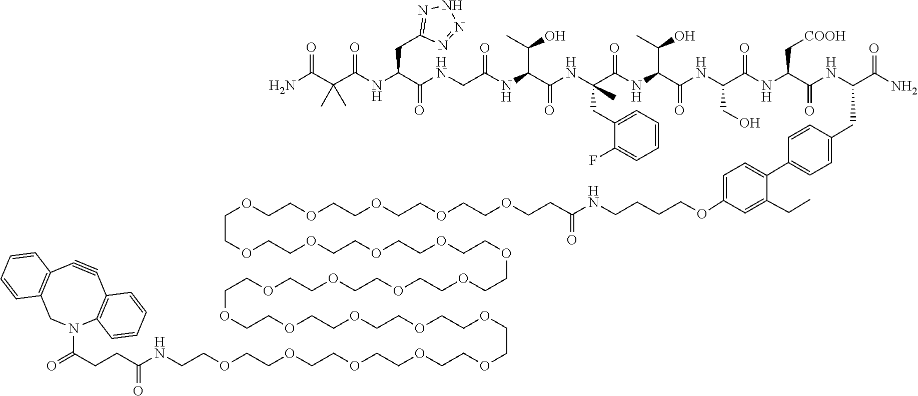 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(N)=O)cc1] |
| LP14′a (SEQ ID NO: 278 and 279 respectively) | 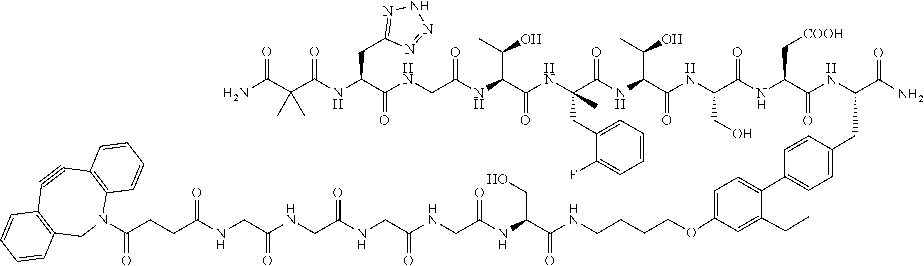 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(N)=O)cc1] |
| LP17′a (SEQ ID NO: 280 and 281 respectively) | 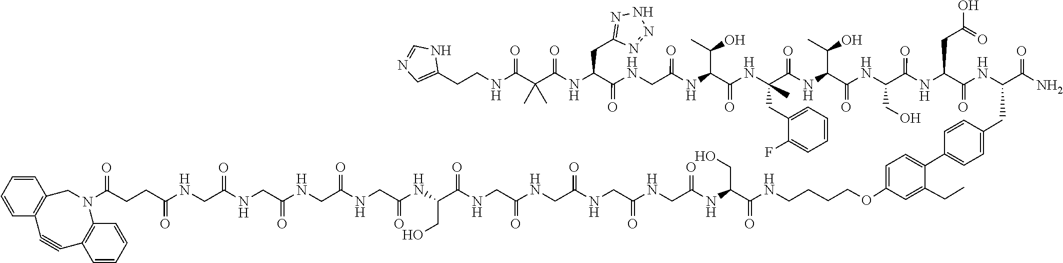 [SMILES: CCc1cc(OCCCCNC(=O)[C@H](CO)NC(=O)CNC(=O)CNC(=O)CNC(=O)CNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP18′a (SEQ ID NO: 282 and 283 respectively) | 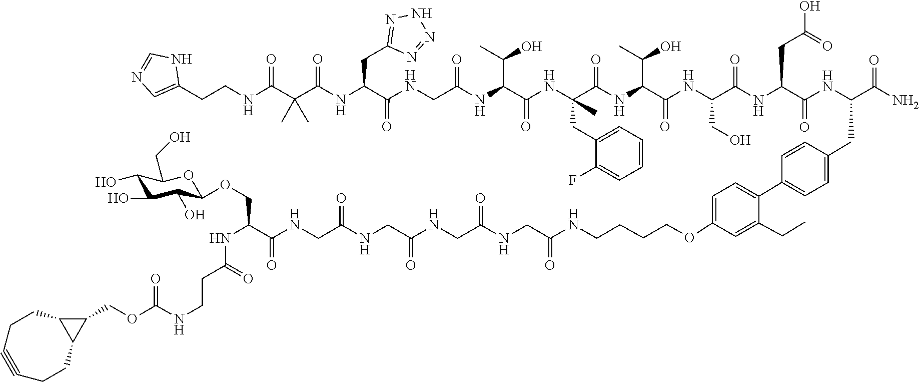 |
| LP19′a (SEQ ID NO: 284 and 285 respectively) | 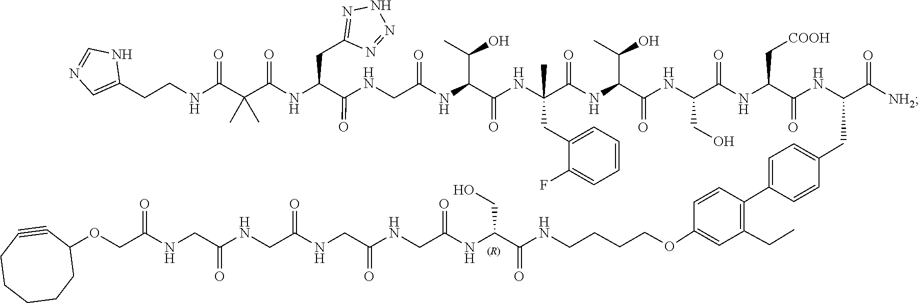 |
| LP20′a (SEQ ID NO: 286 and 287 respectively) | 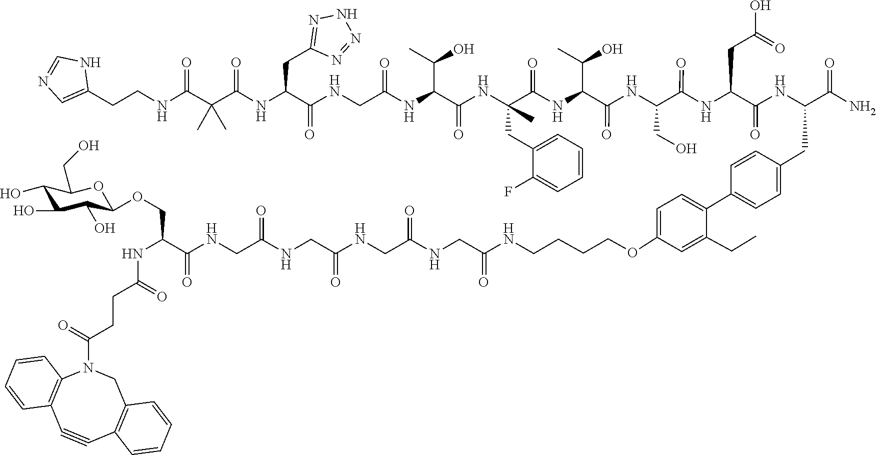 |
| LP22′a (SEQ ID NO: 288) | 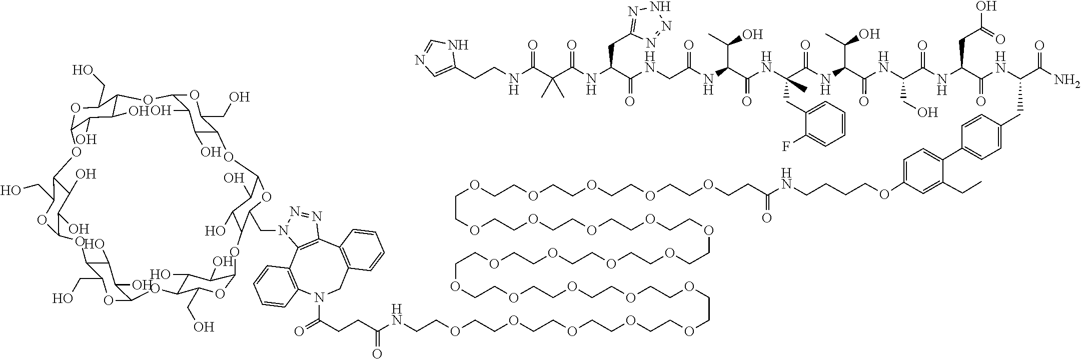 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP24′a (SEQ ID NO: 289) | 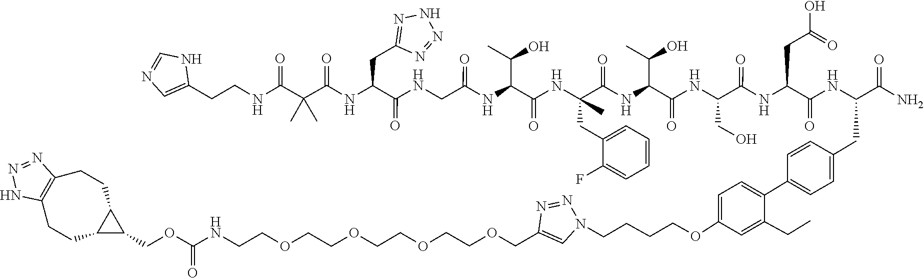 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP25′a (SEQ ID NO: 275) | 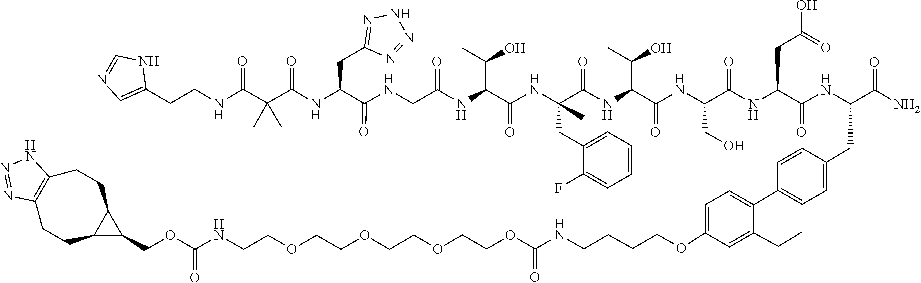 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP26′a (SEQ ID NO: 291 and 292 respectively) | 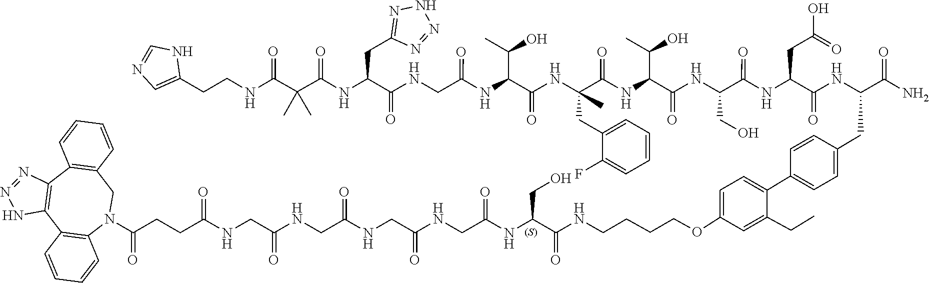 |
| LP27′a (SEQ ID NO: 293) | 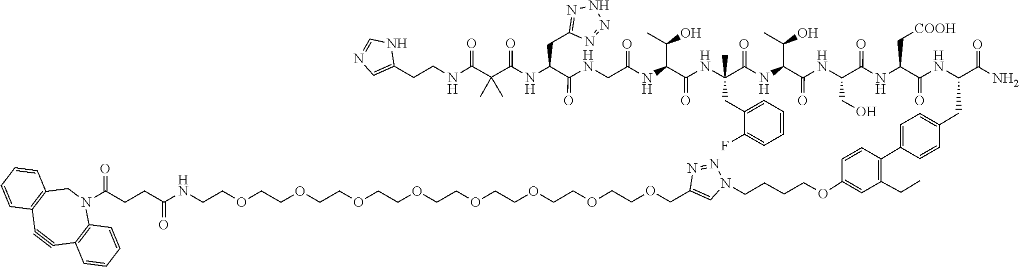 |
| LP28′a (SEQ ID NO: 294) | 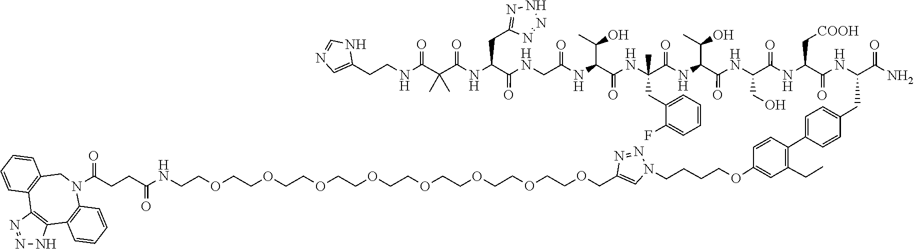 |
| LP29′a (SEQ ID NO: 295) | 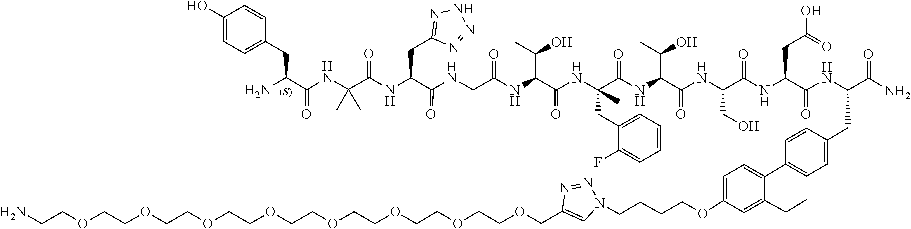 |
| LP30′a (SEQ ID NO: 296) | 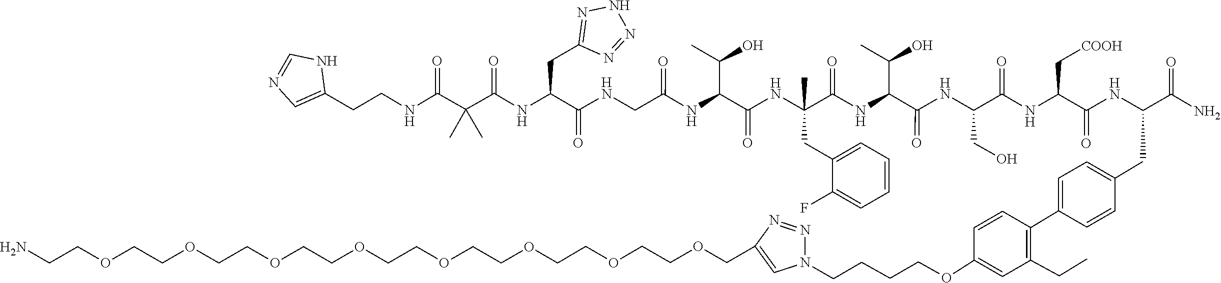 |
| LP31′a (SEQ ID NO: 297) | 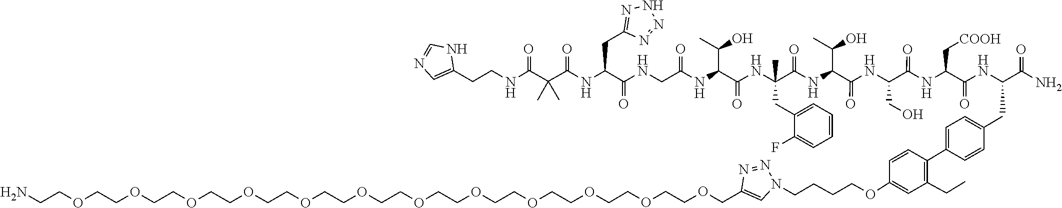 |
| LP33′a (SEQ ID NO: 298) | 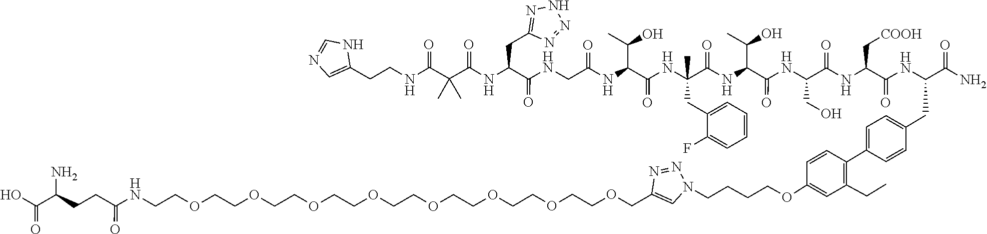 |
| LP34′a (SEQ ID NO: 299 and 300 respectively) | 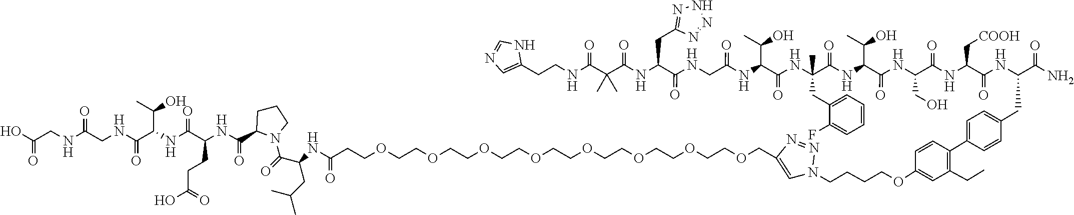 |
| LP35′a (SEQ ID NO: 301 and 302 respectively) | 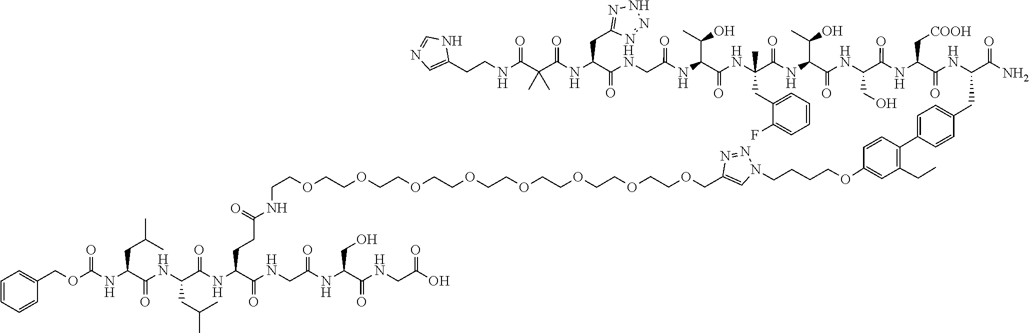 |
| LP36′a (SEQ ID NO: 303 and 304 respectively) | 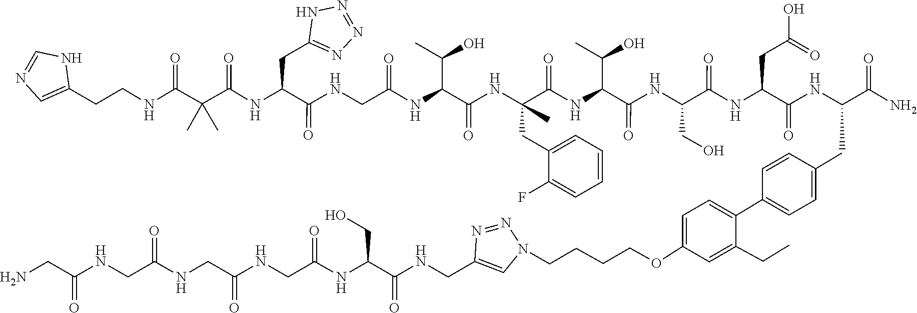 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP37′a (SEQ ID NO: 305 and 306 respectively) | 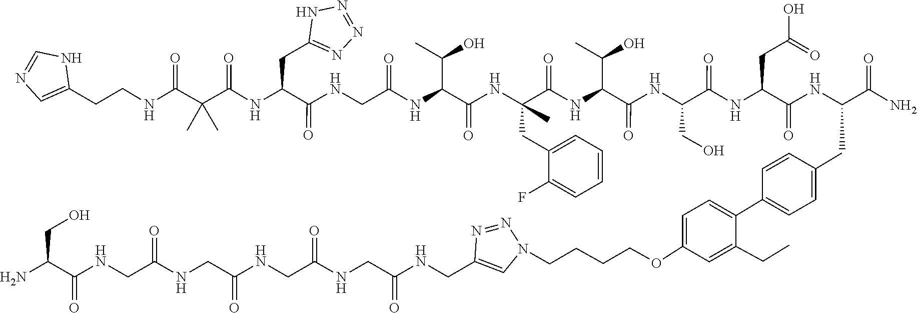 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP38′a (SEQ ID NO: 307 and 353 respectively) | 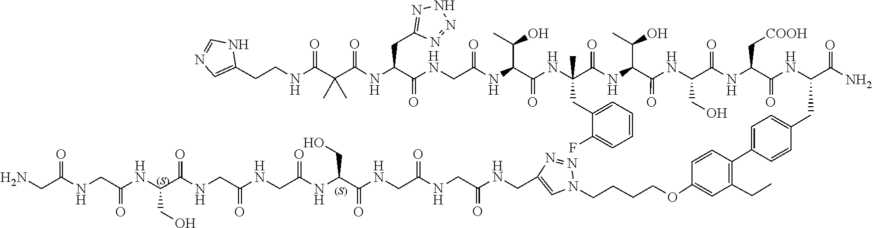 |
| LP39′a (SEQ ID NO: 308 and 309 respectively) | 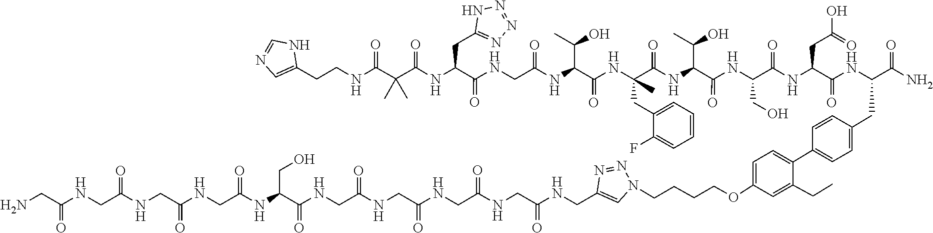 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP40′a (SEQ ID NO: 310 and 311 respectively) | 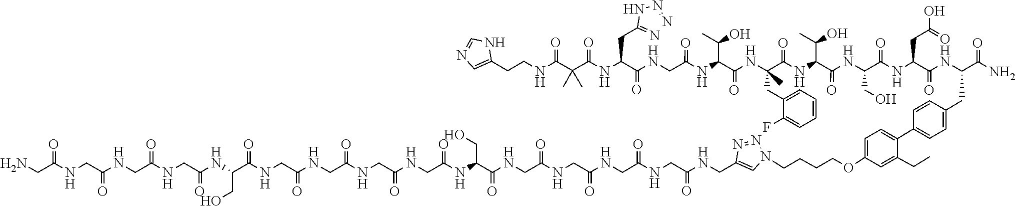 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP41′a (SEQ ID NO: 312 and 313 respectively) | 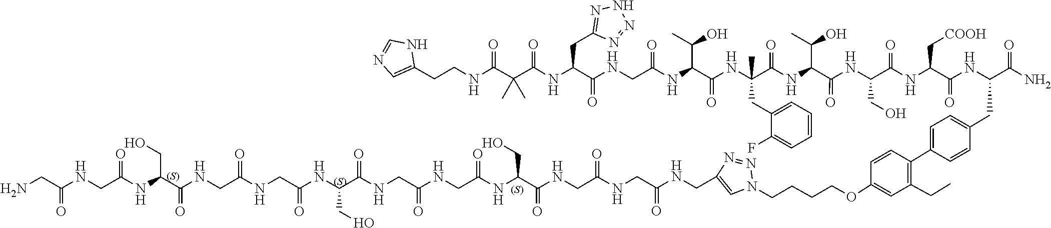 |
| LP42′a (SEQ ID NO: 314 and 315 respectively) | 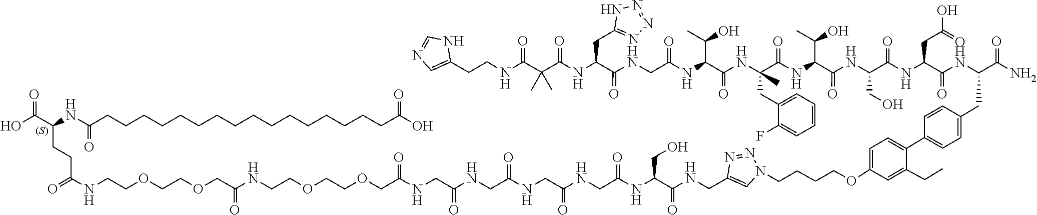 |
| LP43′a (SEQ ID NO: 316 and 317 respectively) | 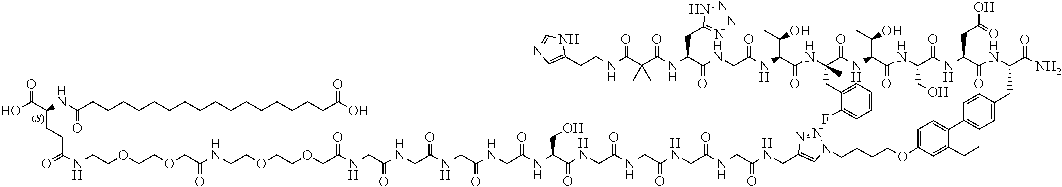 |
| LP44′a (SEQ ID NO: 318) | 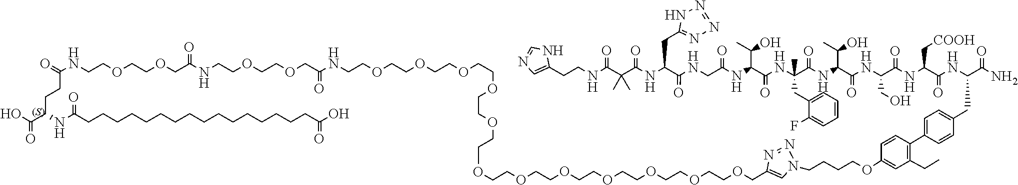 |
| LP45′a (SEQ ID NO: 319) | 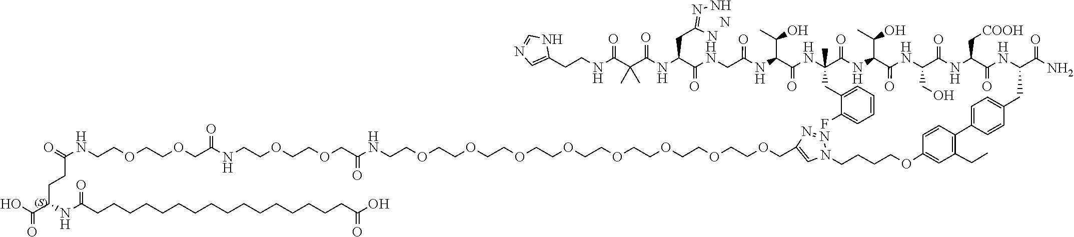 |
| LP46′a (SEQ ID NO: 320) | 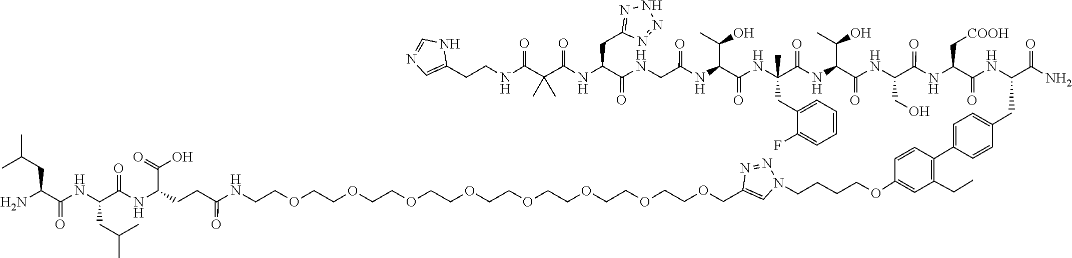 |

or a pharmaceutically acceptable salt thereof.

### Binding Agents

In one embodiment, the effectiveness of the protein-drug conjugate embodiments described herein depend on the selectivity of the binding agent to bind its binding partner. In one embodiment of the present disclosure, the binding agent is any molecule capable of binding with some specificity to a given binding partner. In one embodiment, the binding agent is within a mammal where the interaction can result in a therapeutic use. In an alternative embodiment, the binding agent is in vitro where the interaction can result in a diagnostic use. In some aspects, the binding agent is capable of binding to a cell or cell population.

Suitable binding agents of the present disclosure include proteins that bind to a binding partner. Suitable binding agents include, but are not limited to, antibodies, lymphokines, hormones, growth factors, viral receptors, interleukins, or any other cell binding or peptide binding molecules or substances.

In one embodiment the binding agent is an antibody. In certain embodiments, the antibody is selected from monoclonal antibodies, polyclonal antibodies, antibody fragments (Fab, Fab′, and F(ab)₂, minibodies, diabodies, tribodies, and the like). In certain embodiments, the antibody is the one described in U.S. Patent Application Publication No. 2022/0096648 or U.S. Patent Application Publication No. 2023/0330254, each incorporated by reference in their entirety. Antibodies herein can be humanized using methods described in U.S. Pat. No. 6,596,541 and U.S. Publication No. 2012/0096572, each incorporated by reference in their entirety. In certain embodiments of the protein-drug conjugate compounds of the present disclosure, BA is a humanized monoclonal antibody. For example, BA can be a monoclonal antibody that binds GLP1R.

In the present disclosure, the antibody can be any antibody deemed suitable to the practitioner of skill. In some embodiments, a linker or linker-payload is attached to one or both heavy chains of the antibody or antigen-binding fragment thereof. In some embodiments, a linker or linker-payload is attached to one or both heavy chain variable domains of the antibody or antigen-binding fragment thereof. In some embodiments, a linker or linker-payload is attached to the N-terminus of one or both heavy chain variable domains of the antibody or antigen-binding fragment thereof. In some embodiments, a linker or linker-payload is attached to the N-terminus of both heavy chain variable domains of the antibody or antigen-binding fragment thereof. In some embodiments, a linker or linker-payload is attached to one or both light chains of the antibody or antigen-binding fragment thereof. In some embodiments, a linker or linker-payload is attached to one or both light chain variable domains of the antibody or antigen-binding fragment thereof. In some embodiments, a linker or linker-payload is attached to the N-terminus of one or both light chain variable domains of the antibody or antigen-binding fragment thereof. In some embodiments, a linker or linker-payload is attached to the N-terminus of both light chain variable domains of the antibody or antigen-binding fragment thereof.

In some embodiments, a linker or linker-payload is attached to the C-terminus of one or both heavy chain variable domains of the antibody or antigen-binding fragment thereof. In some embodiments, a linker or linker-payload is attached to the C-terminus of both heavy chain variable domains of the antibody or antigen-binding fragment thereof. In some embodiments, a linker or linker-payload is attached to the C-terminus of one or both light chain variable domains of the antibody or antigen-binding fragment thereof. In some embodiments, a linker or linker-payload is attached to the C-terminus of both light chain variable domains of the antibody or antigen-binding fragment thereof.

In some embodiments, the antibody comprises at least one glutamine residue in at least one polypeptide chain sequence. In certain embodiments, the antibody comprises one or more Gln295 residues. In certain embodiments, the antibody comprises two heavy chain polypeptides, each with one Gln295 residue. In further embodiments, the antibody comprises one or more glutamine residues at a site other than a heavy chain 295. Such antibodies can be isolated from natural sources or engineered to comprise one or more glutamine residues. Techniques for engineering glutamine residues into an antibody polypeptide chain are within the skill of the practitioners in the art. In certain embodiments, a glutamine residue is introduced to the N-terminus of an antibody polypeptide chain. In one embodiment, a glutamine residue is introduced to the N-terminus of one or both heavy chains of the antibody. In one embodiment, a glutamine residue is introduced to the N-terminus of both heavy chains of the antibody. In another embodiment, the glutamine residue is introduced to the N-terminus of one or both light chains of the antibody. In one embodiment, a glutamine residue is introduced to the N-terminus of both light chains of the antibody. In another embodiment, a glutamine residue is introduced to the N-terminus of one or both heavy chains and one or both light chains of the antibody.

In certain embodiments, a glutamine residue is introduced to the C-terminus of an antibody polypeptide chain. In one embodiment, a glutamine residue is introduced to the C-terminus of one or both heavy chains of the antibody. In one embodiment, a glutamine residue is introduced to the C-terminus of both heavy chains of the antibody. In another embodiment, the glutamine residue is introduced to the C-terminus of one or both light chains of the antibody. In one embodiment, a glutamine residue is introduced to the C-terminus of both light chains of the antibody. In another embodiment, a glutamine residue is introduced to the C-terminus of one or both heavy chains and one or both light chains of the antibody.

In certain embodiments, the antibody or antigen-binding fragment thereof has been modified to comprise a TGase recognition tag. Suitable TGase recognition tags include those described herein.

In certain embodiments, the antibody or antigen-binding fragment thereof has been modified to comprise a Q-tag at the N-terminus of one or both antibody light chains. In certain embodiments, the antibody or antigen-binding fragment thereof has been modified to comprise a Q-tag at the N-terminus of both antibody light chains.

In certain embodiments, the antibody or antigen-binding fragment thereof has been modified to comprise a Q-tag at the N-terminus of one or both antibody heavy chains. In certain embodiments, the antibody or antigen-binding fragment thereof has been modified to comprise a Q-tag at the N-terminus of both antibody heavy chains.

In certain embodiments, the antibody or antigen-binding fragment thereof has been modified to comprise a Q-tag at the C-terminus of one or both antibody light chains. In certain embodiments, the antibody or antigen-binding fragment thereof has been modified to comprise a Q-tag at the C-terminus of both antibody light chains.

In certain embodiments, the antibody or antigen-binding fragment thereof has been modified to comprise a Q-tag at the C-terminus of one or both antibody heavy chains. In certain embodiments, the antibody or antigen-binding fragment thereof has been modified to comprise a Q-tag at the C-terminus of both antibody heavy chains.

In certain embodiments, the antibody or antigen-binding fragment thereof is aglycosylated. In certain embodiments, the antibody antigen-binding fragment thereof is deglycosylated. In certain embodiments, the antibody antigen-binding fragment is a Fab fragment.

The antibody can be in any form known to those of skill in the art. In certain embodiments, the antibody comprises a light chain. In certain embodiments, the light chain is a kappa light chain. In certain embodiments, the light chain is a lambda light chain.

In certain embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

The antibody can have binding specificity for any antigen deemed suitable to those of skill in the art. In certain embodiments, the antigen is a transmembrane molecule (e.g., receptor) or a growth factor. Exemplary antigens include, but are not limited to, molecules such as GLP1R.

Some embodiments herein are target specific for therapeutic or diagnostic use.

In some embodiments, the binding agent is an anti-glucagon-like peptide 1 receptor (GLP1R), i.e., an anti-GLP1R antibody, or an antigen-binding fragment thereof. In some embodiments, the binding agent is an anti-GLP1R agonist antibody, or an antigen-binding fragment thereof.

In some embodiments, suitable anti-GLP1R antibodies, including 5A10 and 9A10, are those disclosed in US Publication No. US20060275288A1, which is incorporated herein by reference in its entirety. In some embodiments, suitable anti-GLP1R antibodies include, but not limited to, AB9433-I, h38C2, PA5-111834, NLS1205, MAB2814, EPR21819, and glutazumab.

In some other embodiments, BA is an anti-GLP1R antibody or antigen-binding fragment comprising REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280.

In some embodiments, the anti-GLP1R antibodies and antigen-binding fragments of the present invention are glutaminyl-modified. The term “glutaminyl-modified” antibody, for example, refers to an antibody (e.g., REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280) modified with a payload or a linker-payload (LP) as described in this disclosure, wherein the conjugation of the payload or linker-payload is through at least one covalent linkage from a glutamine side chain of said antibody or a glutamine-Tag (Q-tag)) to a primary amine compound (e.g., a Payload or Linker-Payload) of the present disclosure.

In particular embodiments, the primary amine compound (e.g., a Payload or Linker-Payload) is linked through an amide linkage on the glutamine side chain. In certain embodiments, the glutamine is an endogenous glutamine. In certain embodiments, the glutamine residue is naturally present in a CH2 or CH3 domain of the BA. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags or TGase recognition tag).

In some embodiments, the present disclosure includes anti-GLP1R antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

The present disclosure also provides nucleic acid molecules encoding anti-GLP1R antibodies or portions thereof. Also included within the scope of the present disclosure are recombinant expression vectors capable of expressing a polypeptide comprising a heavy or light chain variable region of an anti-GLP1R antibody. For example, the present disclosure includes recombinant expression vectors comprising any of the nucleic acid molecules mentioned above. Further included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

In another aspect, the disclosure provides a pharmaceutical composition comprising a recombinant human antibody or fragment thereof which specifically binds GLP1R and a pharmaceutically acceptable carrier. In a related aspect, the disclosure includes a composition which is a combination of an anti-GLP1R antibody and a second therapeutic agent. In one embodiment, the second therapeutic agent is any agent that is advantageously combined with an anti-GLP1R antibody. Additional combination therapies and co-formulations involving the anti-GLP1R antibodies of the present disclosure are disclosed elsewhere herein.

In another aspect, the disclosure provides therapeutic methods for targeting cells expressing GLP1R using an anti-GLP1R antibody of the disclosure, wherein the therapeutic methods comprise administering a therapeutically effective amount of a pharmaceutical composition comprising an anti-GLP1R antibody of the disclosure to a subject in need thereof. In some embodiments, the cell is a mammalian cell. In some embodiments, the cell is a human cell. In some embodiments, the cell is a pancreatic cell, a brain cell, a heart cell, a vascular tissue cell, a kidney cell, an adipose tissue cell, a liver cell, or a muscle cell. In one embodiment, the cell is a pancreatic cell or a brain cell.

In some cases, the anti-GLP1R antibodies (or antigen-binding fragments thereof) can be used to enhance GLP1R activity in the cells.

The present disclosure also includes the use of an anti-GLP1R antibody of the disclosure in the manufacture of a medicament for the treatment of a disease or disorder (e.g., type II diabetes and/or obesity) associated with GLP1R-expressing cells. In one aspect, the disclosure relates to a compound comprising an anti-GLP1R antibody or antigen-binding fragment, as disclosed herein, for use in medicine. In one aspect, the disclosure relates to a compound comprising an antibody-drug conjugate (ADC) as disclosed herein, for use in medicine.

In yet another aspect, the disclosure provides monospecific anti-GLP1R antibodies for diagnostic applications, such as, e.g., imaging reagents.

The disclosure further includes an antibody or antigen-binding fragment that competes for binding to human GLP1R with an antibody described herein.

The disclosure furthermore includes an antibody or antigen-binding fragment, wherein the antibody or antigen-binding fragment thereof binds to the same epitope on human GLP1R as an antibody described herein.

In one aspect, the present disclosure provides antibody-drug conjugates comprising an anti-GLP1R antibody or antigen-binding fragment thereof as described above and a therapeutic agent (e.g., a GLP1 peptidomimetic). In some embodiments, the antibody or antigen-binding fragment and the payload are covalently attached via a linker, as discussed above. In various embodiments, the anti-GLP1R antibody or antigen-binding fragment can be any of the anti-GLP1R antibodies or fragments described herein. In some embodiments, the antibody-drug conjugates of the present disclosure are stable in plasma. Plasma stability may be determined using an in vitro or in vivo plasma stability assay. In some embodiments, the antibody-drug conjugates of the present disclosure have a half life of longer than 4 days, longer than 5 days, longer than 6 days, longer than 7 days, longer than 8 days, longer than 9 days, longer than 10 days, longer than 11 days, longer than 12 days, longer than 13 days, longer than 2 weeks, longer than 3 weeks, longer than 4 weeks, about 1 week, about 2 weeks, about 3 weeks, about 4 weeks, about 1 month, about 2 month, about 3 month, about 4 month, about 5 month, about 6 month, between 5-10 days, between 8-12 days, between 10-15 days, between 12-18 days, between 15-20 days, between 20-30 days, between 1-2 weeks, between 2-3 weeks, between 3-4 weeks, between 4-6 weeks, between 5-8 weeks, between 6-10 weeks, between 1-2 months, between 1.5-3 months, between 2-4 months, between 2.5-5 months, between 3-6 months, or between 4-6 months in plasma.

In some embodiments, the antibody-drug conjugates of the present disclosure bind to GLP1R with at least a 10-fold greater affinity than other G protein-coupled receptors (GPCRs). In some embodiments, the antibody-drug conjugates of the present disclosure bind to GLP1R with at least a 20-fold, 50-fold, 100-fold, 500-fold, 1000-fold, 10,000-fold greater affinity than other G protein-coupled receptors (GPCRs). In some embodiments, such antibody-drug conjugates exhibit essentially undetectable binding against GPCRs other than GLP1R. Binding of the antibody-drug conjugates to a target molecule can be measured using a standard binding assay available in the relevant art, such as luciferase reporter assay, surface plasmon resonance assay, enzyme-linked immunosorbent assay (ELISA), radioimmunoassay (RIA), FACS analysis, or Western blot assay. Examples of GPCRs other than GLP1R include, but are not limited to, GIPR, GLP2R, and GCGR.

### Antibody Conjugation

Techniques and linkers for conjugating to residues of an antibody or antigen binding fragment are known in the art. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Lysine conjugation can also proceed through NHS (N-hydroxy succinimide). Linkers can also be conjugated to cysteine residues, including cysteine residues of a cleaved interchain disulfide bond, by forming a carbon bridge between thiols (see, e.g., U.S. Pat. Nos. 9,951,141, and 9,950,076). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36 (Suppl 1): 100). In specific embodiments discussed in more detail below, Site specific conjugation techniques, include glutamine conjugation via transglutaminase (see e.g., Schibli, Angew Chemie Inter Ed. 2010, 49:9995).

Payloads according to the disclosure linked through lysine and/or cysteine, e.g., via a maleimide or amide conjugation, are included within the scope of the present disclosure.

In some embodiments, the protein-drug conjugates of the present disclosure are produced according to a two-step process, where Step 1 is lysine-based linker conjugation, e.g., with an NHS-ester linker, and Step 2 is a payload conjugation reaction (e.g., a 1,3-cycloaddition reaction).

In some embodiments, the protein-drug conjugates of the present disclosure are produced according to a two-step process, where Step 1 is cysteine-based linker conjugation, e.g., with a maleimide linker, and Step 2 is a payload conjugation reaction (e.g., a 1,3-cycloaddition reaction).

In some embodiments, the protein-drug conjugates of the present disclosure are produced according to a two-step process, where Step 1 is transglutaminase-mediated site specific conjugation and Step 2 is a payload conjugation reaction (e.g., a 1,3-cycloaddition reaction).

### Step 1: Transglutaminase Mediated Site Specific Conjugation

In some embodiments, proteins (e.g., antibodies) may be modified in accordance with known methods to provide glutaminyl modified proteins. Techniques for conjugating antibodies and primary amine compounds are known in the art. Site specific conjugation techniques are employed herein to direct conjugation to glutamine using glutamine conjugation via transglutaminase (see e.g., Schibli, Angew Chemie Inter Ed. 2010, 49, 9995).

Primary amine-comprising compounds (e.g., linkers La) of the present disclosure can be conjugated to one or more glutamine residues of a binding agent (e.g., a protein, e.g., an antibody, e.g., an anti-GLP1R antibody) via transglutaminase-based chemo-enzymatic conjugation (see, e.g., Dennler et al., Protein Conjugate Chem. 2014, 25, 569-578, and WO 2017/147542). For example, in the presence of transglutaminase, one or more glutamine residues of an antibody can be coupled to a primary amine linker compound. Briefly, in some embodiments, a binding agent having a glutamine residue (e.g., a Gln295, i.e. Q295 residue) is treated with a primary amine-containing linker La in the presence of the enzyme transglutaminase. In certain embodiments, the binding agent is aglycosylated. In certain embodiments, the binding agent is deglycosylated.

In certain embodiments, the binding agent (e.g., a protein, e.g., an antibody) comprises at least one glutamine residue in at least one polypeptide chain sequence. In certain embodiments, the binding agent comprises two heavy chain polypeptides, each with one Gln295 residue. In further embodiments, the binding agent comprises one or more glutamine residues at a site other than a heavy chain 295.

In some embodiments, a binding agent, such as an antibody, can be prepared by site-directed mutagenesis to insert a glutamine residue at a site without resulting in disabled antibody function or binding. For example, included herein are antibodies bearing Asn297Gln (N297Q) mutation(s) as described herein. In some embodiments, an antibody having a Gln295 residue and/or an N297Q mutation contains one or more additional naturally occurring glutamine residues in their variable regions, which can be accessible to transglutaminase and therefore capable of conjugation to a linker or a linker-payload. An exemplary naturally occurring glutamine residue can be found, e.g., at Q55 of the light chain. In such instances, the binding agent, e.g., antibody, conjugated via transglutaminase can have a higher than expected LAR value (e.g., a LAR higher than 4). Any such antibodies can be isolated from natural or artificial sources.

### Step 2: Payload Conjugation Reaction

In certain embodiments, linkers La according to the present disclosure comprise at least one first reactive group capable of further reaction after transglutamination. In these embodiments, the glutaminyl-modified protein (e.g., antibody) is capable of further reaction with a reactive payload compound P or a reactive linker-payload compound (e.g., Lp-P as disclosed herein), to form a protein-payload conjugate. More specifically, the reactive linker-payload compound Lp-P may comprise a second reactive group that is capable of reacting with the first reactive group of the linker La. In certain embodiments, a first or second reactive group according to the present disclosure comprises a moiety that is capable of undergoing a 1,3-cycloaddition reaction. In certain embodiments, the reactive group is an azide. In certain embodiments, the reactive group comprises an alkyne (e.g., a terminal alkyne, or an internal strained alkyne). In certain embodiments of the present disclosure the reactive group is compatible with the binding agent and transglutamination reaction conditions.

In certain embodiments of the disclosure, linker La molecules comprise a first reactive group. In certain embodiments of the disclosure, linker La molecules comprise more than one reactive group.

In certain embodiments, the reactive linker-payload Lp-P comprises one payload molecule (n=1). In certain other embodiments, the reactive linker-payload Lp-P comprises two or more payload molecules (n≥2).

In certain embodiments of the disclosure, the drug-antibody ratio or DAR is from about 1 to about 30, or from about 1 to about 24, or from about 1 to about 20, or from about 1 to about 16, or from about 1 to about 12, or from about 1 to about 10, or from about 1 to about 8, or about 1, 2, 3, 4, 5, 6, 7, or 8 payload molecules per antibody. In some embodiments, the DAR is from 1 to 30. In some embodiments, the DAR is from 1 to 16. In some embodiments, the DAR is from 1 to 8. In some embodiments, the DAR is from 1 to 6. In certain embodiments, the DAR is from 2 to 4. In some cases, the DAR is from 2 to 3. In certain cases, the DAR is from 0.5 to 3.5. In some embodiments, the DAR is about 1, or about 1.5, or about 2, or about 2.5, or about 3, or about 3.5. In some embodiments, the DAR is 2. In some embodiments, the DAR is 4. In some embodiments, the DAR is 8.

Further details on the antibody preparation and their bioconjugation with the GLP1 peptide mimetics to manufacture antibody-tethered ligands can be found in U.S. Patent Application Publication No. 2022/0096648 and U.S. Patent Application Publication No. 2023/0330254, both of which are incorporated herein by reference in their entirety.

### Therapeutic Formulation and Administration

The present disclosure provides pharmaceutical compositions comprising the protein-drug conjugates of the present disclosure.

In one aspect, the present disclosure provides compositions comprising a population of protein-drug conjugates according to the present disclosure having a drug-antibody ratio (DAR) of about 0.5 to about 30.0.

In one embodiment, the composition has a DAR of about 1.0 to about 2.5.

In one embodiment, the composition has a DAR of about 2.

In one embodiment, the composition has a DAR of about 3.0 to about 4.5.

In one embodiment, the composition has a DAR of about 4.

In one embodiment, the composition has a DAR of about 6.5 to about 8.5.

In one embodiment, the composition has a DAR of about 8.

The compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of a protein-drug conjugate administered to a patient may vary depending upon the age and the size of the patient, target disease, conditions, route of administration, and the like. The suitable dose is typically calculated according to body weight or body surface area. When a protein-drug conjugate of the present disclosure is used for therapeutic purposes in an adult patient, it may be advantageous to intravenously administer the protein-drug conjugate of the present disclosure normally at a single dose of about 0.01 to about 20 mg/kg body weight, more preferably about 0.02 to about 7 mg/kg body weight, about 0.03 to about 5 mg/kg body weight, or about 0.05 to about 3 mg/kg body weight. Depending on the severity of the condition, the frequency and the duration of the treatment can be adjusted. Effective dosages and schedules for administering a protein-drug conjugate may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Protein-Drug Conjugates, Linker-Payloads and Payloads

In another aspect, the compounds (e.g., an antibody-drug conjugate, a linker-payload and/or a payload), disclosed herein are useful, inter alia, for the treatment, prevention and/or amelioration of a disease, disorder or condition in need of such treatment.

In one aspect, the present disclosure provides a method of treating a condition in a subject in need thereof comprising administering to the subject a therapeutically effective amount of a compound (e.g., an antibody-drug conjugate, a linker-payload and/or a payload) according to the disclosure, or the composition comprising any compound according to the present disclosure.

In some embodiments, the compounds (e.g., an antibody-drug conjugate, a linker-payload and/or a payload) disclosed herein are useful for treating any disease or disorder in which stimulation, activation, and/or targeting of GLP1R would be beneficial. In particular, the anti-GLP1R antibody-drug conjugates of the present disclosure can be used for the treatment, prevention, and/or amelioration of any disease or disorder associated with or mediated by GLP1R expression or activity.

In some embodiments, the compounds (e.g., an antibody-drug conjugate, a linker-payload and/or a payload) disclosed herein are useful for treating a GLP1R-associated condition. In some embodiments, the GLP1R-associated condition is Type 1 or Type 2 diabetes mellitus. The administered compound (e.g., an antibody-drug conjugate, a linker-payload and/or a payload) may cause at least one of the following results: induction of insulin secretion, suppression of glucagon release, reduction of blood sugar, improvement of glycemic control, promotion of islet neogenesis, and delay of gastric emptying or potentiation of glucose resistant islets.

In some embodiments, the GLP1R-associated condition is a neurodegenerative disorder, a cognitive disorder, memory disorder, or learning disorder. The neurodegenerative disorder may be, for example, dementia, senile dementia, mild cognitive impairment, Alzheimer-related dementia, Huntington's chores, tardive dyskinesia, hyperkinesias, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis, glaucoma, and Alzheimer's disease.

In some embodiments, the GLP1R-associated condition is a liver disease. The liver disease may be, for example, non-alcoholic fatty liver disease (NAFLD), fatty liver, non-alcoholic steatohepatitis (NASH), and cirrhosis.

In some embodiments, the GLP1R-associated condition is a coronary artery disease. The coronary artery disease may be, for example, cardiomyopathy and myocardial infarction.

In some embodiments, the GLP1R-associated condition is an obesity.

In some embodiments, the GLP1R-associated condition is a kidney disease. The kidney disease may be, for example, hypertension or chronic kidney failure.

In some embodiments, the GLP1R-associated condition is an eating disorder. The eating disorder may be, for example, binge eating.

Without wishing to be bound by theory, the compounds (e.g., an antibody-drug conjugate, a linker-payload and/or a payload) disclosed herein may be employed to attenuate the effects of apoptosis-mediated degenerative diseases of the central nervous system such as Alzheimer's Disease, Creutzfeld-Jakob Disease and bovine spongiform encephalopathy, chronic wasting syndrome and other prion mediated apoptotic neural diseases (see, e.g., Perry and Grieg (2004) Current Drug Targets 6:565-571). Administration of a compound (e.g., an antibody-drug conjugate, a linker-payload and/or a payload) disclosed herein may also lead to down-modulation of PAPP and thereby ameliorate A3 mono- or oligomer-mediated pathologies associated with Alzheimer's Disease (see, e.g., Perry et al. (2003) Journal of Neuroscience Research 72:603-612).

It is also contemplated that the compounds (e.g., an antibody-drug conjugate, a linker-payload and/or a payload) disclosed herein may be used to improve learning and memory, for example, by enhancing neuronal plasticity and facilitation of cellular differentiation (see, During et al. (2003) Nature Medicine 9:1173-1179). Further, the compounds (e.g., an antibody-drug conjugate, a linker-payload and/or a payload) disclosed herein may also be used to preserve dopamine neurons and motor function in Morbus Parkinson (see, e.g., Greig et al. (2005) Abstract 897.6, Society for Neuroscience, Washington, D.C.).

In some embodiments, the compounds (e.g., an antibody-drug conjugate, a linker-payload and/or a payload) disclosed herein may also be used to treat a metabolic disorder. The metabolic disorder may be, for example, obesity, dyslipidemia, metabolic syndrome X, and pathologies emanating from islet insufficiency.

Additional diseases that may be treated by a compound (e.g., an antibody-drug conjugate, a linker-payload and/or a payload) of the present disclosure include autoimmune diseases, in particular, those associated with inflammation, including, but not limited to, autoimmune diabetes, adult onset diabetes, morbid obesity, Metabolic Syndrome X, and dyslipidemia. For example, the anti-GLP1R antibody-drug conjugate can be employed as a growth factor for the promotion of islet growth in persons with autoimmune diabetes. The compounds (e.g., an antibody-drug conjugate, a linker-payload and/or a payload) described herein may also be useful in the treatment of congestive heart failure.

In one aspect, the present disclosure provides a method of selectively targeting an antigen (e.g., GLP1R) on a surface of a cell with a compound. In one embodiment, the method of selectively targeting an antigen (e.g., GLP1R) on a surface of a cell with a compound comprises linking the compound to a targeted antibody. In one embodiment, the compound is a payload as described above. In one embodiment, the cell is a mammalian cell. In one embodiment, the cell is a human cell. In one embodiment, the cell is a pancreatic cell or a brain cell. In another embodiment, the cell is a heart cell, a vascular tissue cell, a kidney cell, an adipose tissue cell, a liver cell, or a muscle cell.

In one aspect, the present disclosure provides a method of enhancing GLP1R activity in an individual in need thereof comprising administering to the individual an effective amount of the compound of any of the embodiments described herein, the composition described herein, or the dosage form described herein.

In certain embodiments, the present disclosure also includes a method of lowering blood glucose levels in an individual in need thereof comprising administering to the individual an effective amount of the compound of any of the embodiments described herein, the composition described herein, or the dosage form described herein.

In certain embodiments, the present disclosure also includes a method of lowering body weight in an individual in need thereof comprising administering to the individual an effective amount of the compound of any of the embodiments described herein, the composition described herein, or the dosage form described herein.

In certain embodiments, the present disclosure also includes the use of a compound (e.g., an antibody-drug conjugate, a linker-payload and/or a payload) of the present disclosure in the manufacture of a medicament for the treatment of a disease or disorder (e.g., cancer) related to or caused by GLP1R-expressing cells. In one aspect, the present disclosure relates to a protein-drug conjugate comprising an anti-GLP1R antibody or antigen-binding fragment, as disclosed herein, for use in medicine. In one aspect, the present disclosure relates to a compound comprising an antibody-drug conjugate (ADC) as disclosed herein, for use in medicine.

### Combination Therapies and Formulations

The present disclosure provides methods which comprise administering a pharmaceutical composition comprising any of the exemplary protein-drug conjugates (e.g., antibody-drug conjugates), linker-payloads, and payloads described herein in combination with one or more additional therapeutic agents.

Exemplary additional therapeutic agents that may be combined with or administered in combination with protein-drug conjugates (e.g., antibody-drug conjugates), linker-payloads, and payloads of the present disclosure include, other GLP1R agonists (e.g., an anti-GLP1R antibody or a small molecule agonist of GLP1R or an anti-GLP1R antibody-drug conjugate). Non-limiting examples of GLP1R agonists include exenatide (Byetta, Bydureon), liraglutide (Victoza, Saxenda), lixisenatide (Lyxumia in Europe, Adlyxin in the United States), albiglutide (Tanzeum), dulaglutide (Trulicity), semaglutide (Ozempic), and taspoglutide.

Exemplary additional therapeutic agents may include dual or triple-agonists, including GLP1R/GIPR dual agonists, such as GLP1R/GCGR dual agonists, GLP1R/GIPR/GCGR triple-agonists.

Other agents that may be beneficially administered in combination with the protein-drug conjugates (e.g., antibody-drug conjugates), linker-payloads, and payloads of the disclosure include those that are useful in the treatment of diabetes (e.g., type II diabetes), obesity, and/or other related metabolic diseases.

In some embodiments, the additional therapeutic agent is an antidiabetic agent. Any suitable antidiabetic agents can be used. Non-limiting examples of antidiabetic agents include insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones or TZDs (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, glucokinase activators, squalene synthase inhibitors, other lipid lowering agents and aspirin. In some such embodiments, the antidiabetic agent is an oral antidiabetic agents (OAA) such as metformin, acarbose, or TZDs. In some such embodiments, the antidiabetic agent is metformin.

In some embodiments, the GLP1R agonist and one or more antidiabetic agents may be formulated into the same dosage form, such as a solution or suspension for parenteral administration.

The additional therapeutically active component(s) may be administered just prior to, concurrent with, or shortly after the administration of a compound of the present disclosure; (for purposes of the present disclosure, such administration regimens are considered the administration of an antigen-binding molecule “in combination with” an additional therapeutically active component).

The present disclosure includes pharmaceutical compositions in which protein-drug conjugates (e.g., antibody-drug conjugates), linker-payloads and/or payloads of the present disclosure are co-formulated with one or more of the additional therapeutically active component(s) as described elsewhere herein.

### Administration Regimens

According to certain embodiments of the present disclosure, multiple doses of a protein-drug conjugate (e.g., an anti-GLP1R antibody-drug conjugate), linker-payload, and/or a payload may be administered to a subject over a defined time course. The methods according to this aspect of the disclosure comprise sequentially administering to a subject multiple doses of a protein-drug conjugate (e.g., an anti-GLP1R antibody-drug conjugate), linker-payload, and/or a payload of the disclosure. As used herein, “sequentially administering” means that each dose of a protein-drug conjugate (e.g., an anti-GLP1R antibody-drug conjugate), linker-payload, and/or a payload is administered to the subject at a different point in time, e.g., on different days separated by a predetermined interval (e.g., hours, days, weeks, or months). The present disclosure includes methods which comprise sequentially administering to the patient a single initial dose of a protein-drug conjugate (e.g., an anti-GLP1R antibody-drug conjugate), linker-payload, and/or a payload, followed by one or more secondary doses of the protein-drug conjugate (e.g., an anti-GLP1R antibody-drug conjugate), linker-payload, and/or payload, and optionally followed by one or more tertiary doses of the a protein-drug conjugate (e.g., an anti-GLP1R antibody-drug conjugate), linker-payload, and/or payload.

The terms “initial dose,” “secondary doses,” and “tertiary doses,” refer to the temporal sequence of administration of the protein-drug conjugate (e.g., an anti-GLP1R antibody-drug conjugate), linker-payload, and/or payload of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the protein-drug conjugate (e.g., an anti-GLP1R antibody-drug conjugate), linker-payload, and/or payload, but generally may differ from one another in terms of frequency of administration. In certain embodiments, however, the amount of the protein-drug conjugate (e.g., an anti-GLP1R antibody-drug conjugate), linker-payload, and/or payload contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3 3/2, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of a protein-drug conjugate (e.g., an anti-GLP1R antibody-drug conjugate), linker-payload, and/or payload which is administered to a patient prior to the administration of the very next dose in the sequence with no intervening doses.

The methods according to this aspect of the disclosure may comprise administering to a patient any number of secondary and/or tertiary doses of a protein-drug conjugate (e.g., an anti-GLP1R antibody-drug conjugate), linker-payload, and/or payload. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, the binding agent or the antibody comprises the following specific protein or nucleotide sequences, respectively.

REGN7990
Heavy chain variable region (HCVR; V_{H})-nucleotide sequence
(SEQ ID NO: 23)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA
Heavy chain variable region (HCVR; V_{H})-amino acid sequence
(SEQ ID NO: 24)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS
CDR-H1-nucleotide sequence
(SEQ ID NO: 25)
GGA TTC ACC TTT AGC AGC TAT GCC
CDR-H1-amino acid sequence
(SEQ ID NO: 26)
G F T F S S Y A
CDR-H2-nucleotide sequence
(SEQ ID NO: 27)
ATT AGC GGT AGT GGT GGC AGC ACA
CDR-H2-amino acid sequence
(SEQ ID NO: 28)
I S G S G G S T
CDR-H3-nucleotide sequence
(SEQ ID NO: 29)
GCG AAA GGC CTT ATA GCA CCT CGT CCG ATG GGC TTT GAC TAC
CDR-H3-amino acid sequence
(SEQ ID NO: 30)
A K G L I A P R P M G F D Y
Light chain variable region (LCVR; V_{L})-nucleotide sequence
(SEQ ID NO: 31)
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGCAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A
Light chain variable region (LCVR; V_{L})-amino acid sequence
(SEQ ID NO: 32)
DIQMTQSPSSVSASVGDRVTITCRASQGINSWLAWYQQKPGKAPKLLIYAASSLQSGVPSRFSGSGSGTDFTLTISSLQP
EDFATYYCHQADSFPYTFGQGTKLEIK
CDR-L1-nucleotide sequence
(SEQ ID NO: 33)
CAG GGT ATT AAC AGC TGG
CDR-L1-amino acid sequence
(SEQ ID NO: 34)
Q G I N S W
CDR-L2-nucleotide sequence
GCT GCA TCC
CDR-L2-amino acid sequence
A A S
CDR-L3-nucleotide sequence
(SEQ ID NO: 37)
CAC CAG GCT GAC AGT TTC CCG TAC ACT
CDR-L3-amino acid sequence
(SEQ ID NO: 38)
H Q A D S F P Y T
Heavy chain-nucleotide sequence
(SEQ ID NO: 39)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAATGA
Heavy chain-amino acid sequence
(SEQ ID NO: 40)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
Light chain-nucleotide sequence
(SEQ ID NO: 41)
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGCAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG
Light chain-amino acid sequence
(SEQ ID NO: 42)
LLQGSGDIQMTQSPSSVSASVGDRVTITCRASQGINSWLAWYQQKPGKAPKLLIYAASSLQSGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
REGN15869
Heavy chain variable region (HCVR; V_{H})-nucleotide sequence
(SEQ ID NO: 43)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA
Heavy chain variable region (HCVR; V_{H})-amino acid sequence
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS
(SEQ ID NO: 44)
CDR-H1-nucleotide sequence
(SEQ ID NO: 45)
GGA TTC ACC TTT AGC AGC TAT GCC
CDR-H1-amino acid sequence
(SEQ ID NO: 46)
G F T F S S Y A
CDR-H2-nucleotide sequence
(SEQ ID NO: 47)
ATT AGC GGT AGT GGT GGC AGC ACA
CDR-H2-amino acid sequence
(SEQ ID NO: 48)
I S G S G G S T
CDR-H3-nucleotide sequence
(SEQ ID NO: 49)
GCG AAA GGC CTT ATA GCA CCT CGT CCG ATG GGC TTT GAC TAC
CDR-H3-amino acid sequence
(SEQ ID NO: 50)
A K G L I A P R P M G F D Y
Light chain variable region (LCVR; V_{L})-nucleotide sequence
(SEQ ID NO: 51)
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGGAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A
Light chain variable region (LCVR; V_{L})-amino acid sequence
(SEQ ID NO: 52)
DIQMTQSPSSVSASVGDRVTITCRASQGINSWLAWYQQKPGKAPKLLIYAASSLESGVPSRFSGSGSGTDFTLTISSLQP
EDFATYYCHQADSFPYTFGQGTKLEIK
CDR-L1-nucleotide sequence
(SEQ ID NO: 53)
CAG GGT ATT AAC AGC TGG
CDR-L1-amino acid sequence
(SEQ ID NO: 54)
Q G IN S W
CDR-L2-nucleotide sequence
GCT GCA TCC
CDR-L2-amino acid sequence
A A S
CDR-L3-nucleotide sequence
(SEQ ID NO: 57)
CAC CAG GCT GAC AGT TTC CCG TAC ACT
CDR-L3-amino acid sequence
(SEQ ID NO: 58)
H Q A D S FP Y T
Heavy chain-nucleotide sequence
(SEQ ID NO: 59)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAATGA
Heavy chain-amino acid sequence
(SEQ ID NO: 60)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
Light chain-nucleotide sequence
(SEQ ID NO: 61)
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGGAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG
Light chain-amino acid sequence
(SEQ ID NO: 62)
LLQGSGDIQMTQSPSSVSASVGDRVTITCRASQGINSWLAWYQQKPGKAPKLLIYAASSLESGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
REGN18121
Heavy chain variable region (HCVR; V_{H})-nucleotide sequence
(SEQ ID NO: 43)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA
Heavy chain variable region (HCVR; V_{H})-amino acid sequence
(SEQ ID NO: 44)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS
CDR-H1-nucleotide sequence
(SEQ ID NO: 45)
GGA TTC ACC TTT AGC AGC TAT GCC
CDR-H1-amino acid sequence
(SEQ ID NO: 46)
G F T F SS Y A
CDR-H2-nucleotide sequence
(SEQ ID NO: 47)
ATT AGC GGT AGT GGT GGC AGC ACA
CDR-H2-amino acid sequence
(SEQ ID NO: 48)
I S G S G G S T
CDR-H3-nucleotide sequence
(SEQ ID NO: 49)
GCG AAA GGC CTT ATA GCA CCT CGT CCG ATG GGC TTT GAC TAC
CDR-H3-amino acid sequence
(SEQ ID NO: 50)
A K G L I A P R P M G F D Y
Light chain variable region (LCVR; V_{L})-nucleotide sequence
(SEQ ID NO: 51)
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGGAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A
Light chain variable region (LCVR; V_{L})-amino acid sequence
(SEQ ID NO: 52)
DIQMTQSPSSVSASVGDRVTITCRASQGINSWLAWYQQKPGKAPKLLIYAASSLESGVPSRFSGSGSGTDFTLTISSLQP
EDFATYYCHQADSFPYTFGQGTKLEIK
CDR-L1-nucleotide sequence
(SEQ ID NO: 53)
CAG GGT ATT AAC AGC TGG
CDR-L1-amino acid sequence
(SEQ ID NO: 54)
Q G I N S W
CDR-L2-nucleotide sequence
GCT GCA TCC
CDR-L2-amino acid sequence
A A S
CDR-L3-nucleotide sequence
(SEQ ID NO: 57)
CAC CAG GCT GAC AGT TTC CCG TAC ACT
CDR-L3-amino acid sequence
(SEQ ID NO: 58)
H Q A D S F P Y T
Heavy chain-nucleotide sequence
(SEQ ID NO: 64)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAA
Heavy chain-amino acid sequence
(SEQ ID NO: 63)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLG
Light chain-nucleotide sequence
(SEQ ID NO: 61)
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGGAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG
Light chain-amino acid sequence
(SEQ ID NO: 62)
LLQGSGDIQMTQSPSSVSASVGDRVTITCRASQGINSWLAWYQQKPGKAPKLLIYAASSLESGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
REGN18123
(SEQ ID NO: 43)
Heavy chain variable region (HCVR; V_{H})-nucleotide sequence
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCA
Heavy chain variable region (HCVR; V_{H})-amino acid sequence
(SEQ ID NO: 44)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSS
CDR-H1-nucleotide sequence
(SEQ ID NO: 45)
GGA TTC ACC TTT AGC AGC TAT GCC
CDR-H1-amino acid sequence
(SEQ ID NO: 46)
G F T F S S Y A
CDR-H2-nucleotide sequence
(SEQ ID NO: 47)
ATT AGC GGT AGT GGT GGC AGC ACA
CDR-H2-amino acid sequence
(SEQ ID NO: 48)
I S G S G G S T
CDR-H3-nucleotide sequence
(SEQ ID NO: 49)
GCG AAA GGC CTT ATA GCA CCT CGT CCG ATG GGC TTT GAC TAC
CDR-H3-amino acid sequence
(SEQ ID NO: 50)
A K G L I A P R P M G F D Y
Light chain variable region (LCVR; V_{L})-nucleotide sequence
(SEQ ID NO: 51)
GACATCCAGATGACCCAGTCTCCATCTTCCGTGTCTGCATCTGTAGGAGACAGAGTCACCATCACTTGTCGGGCGAGTCA
GGGTATTAACAGCTGGTTAGCCTGGTATCAGCAGAAACCTGGGAAAGCCCCTAAGCTCCTGATCTATGCTGCATCCAGTT
TGGAAAGTGGGGTCCCATCAAGGTTCAGCGGCAGTGGATCTGGGACAGATTTCACTCTCACCATCAGCAGCCTGCAGCCT
GAAGATTTTGCAACTTACTATTGTCACCAGGCTGACAGTTTCCCGTACACTTTTGGCCAGGGGACCAAGCTGGAGATCAA
A
Light chain variable region (LCVR; V_{L})-amino acid sequence
(SEQ ID NO: 52)
DIQMTQSPSSVSASVGDRVTITCRASQGINSWLAWYQQKPGKAPKLLIYAASSLESGVPSRFSGSGSGTDFTLTISSLQP
EDFATYYCHQADSFPYTFGQGTKLEIK
CDR-L1-nucleotide sequence
(SEQ ID NO: 53)
CAG GGT ATT AAC AGC TGG
CDR-L1-amino acid sequence
(SEQ ID NO: 54)
Q G I N S W
CDR-L2-nucleotide sequence
GCT GCA TCC
CDR-L2-amino acid sequence
A A S
CDR-L3-nucleotide sequence
(SEQ ID NO: 57)
CAC CAG GCT GAC AGT TTC CCG TAC ACT
CDR-L3-amino acid sequence
(SEQ ID NO: 58)
H Q A D S FP Y T
Heavy chain-nucleotide sequence
(SEQ ID NO: 66)
GAGGTGCAGCTGGTGGAGTCTGGGGGAGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGCTGGAATGGGTCTCAGCTATTAGCGGTA
GTGGTGGCAGCACATACTACGCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGGCCTTATAGCACCTCGTCCGATGGG
CTTTGACTACTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGT
Heavy chain-amino acid sequence
(SEQ ID NO: 65)
EVQLVESGGGLVQPGGSLRLSCAASGFTFSSYAMSWVRQAPGKGLEWVSAISGSGGSTYYADSVKGRFTISRDNSKNTLY
LQMNSLRAEDTAVYYCAKGLIAPRPMGFDYWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSL
Light chain-nucleotide sequence
(SEQ ID NO: 61)
CTGCTGCAAGGCTCTGGCGACATCCAGATGACCCAGTCTCCATCTTCCGTGTCTGCATCTGTAGGAGACAGAGTCACCAT
CACTTGTCGGGCGAGTCAGGGTATTAACAGCTGGTTAGCCTGGTATCAGCAGAAACCTGGGAAAGCCCCTAAGCTCCTGA
TCTATGCTGCATCCAGTTTGGAAAGTGGGGTCCCATCAAGGTTCAGCGGCAGTGGATCTGGGACAGATTTCACTCTCACC
ATCAGCAGCCTGCAGCCTGAAGATTTTGCAACTTACTATTGTCACCAGGCTGACAGTTTCCCGTACACTTTTGGCCAGGG
GACCAAGCTGGAGATCAAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAAAGA
GCTTCAACAGGGGAGAGTGTTAG
Light chain-amino acid sequence
(SEQ ID NO: 62)
LLQGSGDIQMTQSPSSVSASVGDRVTITCRASQGINSWLAWYQQKPGKAPKLLIYAASSLESGVPSRFSGSGSGTDFTLT
ISSLQPEDFATYYCHQADSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC

### EXAMPLES

The following examples illustrate specific aspects of the instant description. The examples should not be construed as limiting, as the examples merely provide specific understanding and practice of the embodiments and their various aspects.

The abbreviations used in the Examples and throughout the specification are shown in Table 1.

TABLE 1
Abbreviations

| | |
|---|---|
| DCM | Dichlormethane |
| DIEA | Diisopropylethylamine |
| DIC | N,N′-Diisopropylcarbodiimide |
| DMF | N,N-Dimethylformamide |
| Oxyma | Ethyl -2-cyano-2-hydroxyiminoacetate |
| MeOH | Methanol |
| TFA | Trifluoroacetic acid |
| MPA, 3-MPA | 3-Mercaptopropionicacid |
| TIS | Triisopropylsilane |
| Pip | Piperidine |
| NH₂NH₂•H₂O | Hydrazine Hydrate |
| EtPhS | Ethylsulfanylbenzene |
| ACN | Acetonitrile |
| SPPS | Solid Phase Peptide Synthesis |
| NMR | Bruker 400 MHZ |
| LCMS | Agilent HPLC 1260-MSD(G6125C) |
| UPLC | Agilent UPLC 1290 |
| IC | Thermo Dionex ICS-5000 + DP |
| MTBE | Methyl tert-Butyl Ether |
| PE | Phenyl Ether |
| EA | Ethyl Acetate |
| MTG | Microbial Transglutaminase |
| PS80 | Polysorbate 80 |

The following amino acids were used in the Examples:

TABLE 2
Amino Acids

| Name | AAs | Structure |
|---|---|---|
| Fmoc- AA1 | Fmoc- AA1-OH | 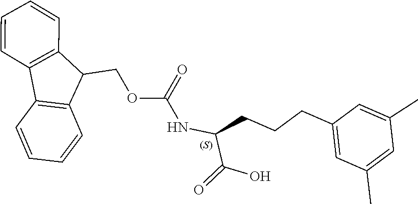 |
| Fmoc- AA2 | Fmoc-AA2 (Boc-Linker)- OH | 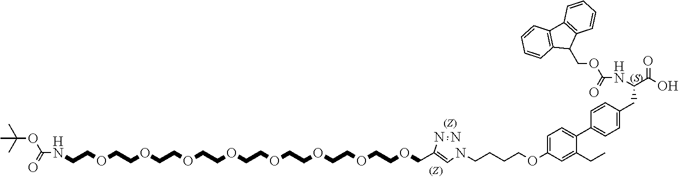 |
| New Fmoc- AA2 | New Fmoc- AA2, M6508/ Cpd # 542 | 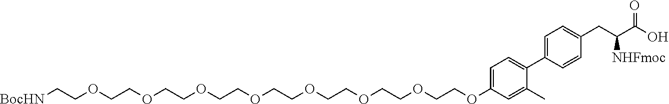 |
| Fmoc- AA3 | Fmoc-Asp (OtBu)-OH | 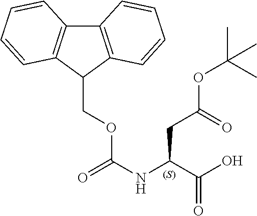 |
| Fmoc- AA4 | Fmoc- Ser(Trt)-OH | 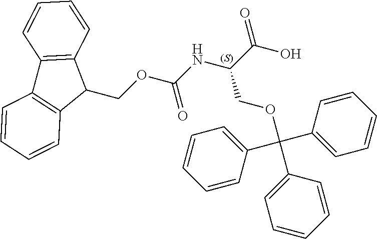 |
| Fmoc- AA5 and Fmoc- AA7 | Fmoc- Thr-OH | 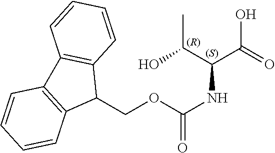 |
| Fmic- AA6 | Fmoc-α- Me-Phe (2-F)-OH | 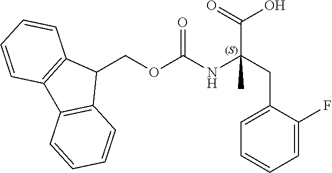 |
| Fmoc- AA8 | Fmoc- Gly-OH | 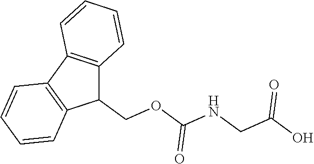 [SMILES: O=C(O)CNC(=O)OCC1c2ccccc2-c2ccccc21] |
| Fmoc- AA9 | Fmoc- AA9-OH | 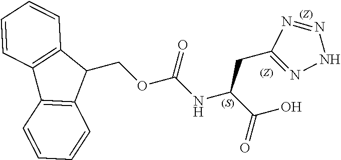 |
| Fmoc- AA10 | Fmoc- AA10-OH | 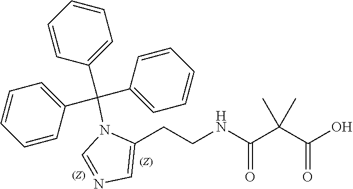 [SMILES: CC(C)(C(=O)O)C(=O)NCCc1cncn1C(c1ccccc1)(c1ccccc1)c1ccccc1] |

The following instruments and experimental conditions were used in the Examples:

TABLE 3
Instrument Names and Conditions

| Instrument Names | Conditions | |
|---|---|---|
| LCMS(Agilent HPLC | Column | Xbridge C18 2.1 × 30 mm, 5 um |
| 1260-G6125C) | Detector | vwd(220 nm&254 nm)/MS(ESI) |
| HPLC(Agilent HPLC | Column | Gemini-NX C18 5 um 110 A 150*4.6 mm |
| 1260) | Detector | vwd(220 nm&254 nm) |
| UPLC(Agilent HPLC | Column | Kinetex 1.7 um EVO C18 100 A |
| 1290) | | LC Column 150 × 2.1 mm |
| | | ACQUITY UPLC BEH C18, 1.7 um 2.1*100 mm |
| | Detector | DAD(220 nm&254 nm) |
| Gilson GX-281 | Column | Gemini, 110 A + luna, c18, 10 um, 100 A |
| | Detector | PDA (220 nm&254 nm) |
| Qing Bohua | Column | DAC-100, Luna C18 10 um, 100 A, 10 cm |
| | | (diameter) * 25 cm (length) |
| | Detector | PDA(220 nm&254 nm) |
| Thermo Dionex | Column | Thermo Dionex IonPac AS19 4 mm*25 cm |
| ICS2100 | Detector | Conductivity Detector |

| | |
|---|---|
| NMR(Bruker_400 MHz) | HNMR&FNMR&CNMR |

### Example 1. General Procedure for Preparation of Peptide Acids

Peptides were synthesized using standard Fmoc chemistry. Peptide acids (e.g. a peptide with a C-terminal acid) were synthesized using standard SPPS with chlorotrityl chloride (CTC) resin with substitution (Sub) 0.7 mmol/g. And peptide amides (e.g. a peptide with a C-terminal amide) were synthesized using a Rink amide resin as known in the art.

### 1.1 Resin Preparation

To the CTC resin (1.00 eq., Sub 0.7 mmol/g) was added a solution of Fmoc-AAn-OH (1.0 eq.), DIEA (2.0 eq) in DCM (7 mL/g Resin) and the mixture was agitated with N₂ for 2 hours. Then MeOH (1 mL/g Resin) was added to the resin, capping the residual reactive site of the resin for 30 minutes. Then the mixture was filtered to get the resin.

### 1.2 De-protection

The Fmoc group of the AAn on the resin was removed by using 20% piperidine in DMF in 2× volume of peptide resin. This reaction was carried out for 20 minutes.

### 1.3 Coupling Condition

The amino acid (AA2-1.5 eq, AA3/AA4/AA5/AA7/AA8-5.0 eq, AA6/AA9-3.0 eq, AA10-2.5 eq) was coupled using DIC/Oxyma (same as amino acid equivalent). The reaction was carried out for periods ranging from 40 minutes to 16 hours depending on the type of amino acid (AA2-120 min, AA3/AA4/AA5/AA6/AA8/AA9/AA10-40 min, AA7-16 hrs).

### 1.4 Cleavage

After the completion of SPPS, the peptide resin was cleaved using cleavage buffer (2.5% H₂O, 7.5% 3-MPA, 2.5% TIS, and 87.5% TFA) for 2 hours. The amount of cleavage buffer used was usually 10 mL/g peptide resin. The cleavage buffer filtrate was concentrated to 30% of the original volume and then the desired peptide was precipitated using cold isopropyl ether (20 times the volume of post-concentrated cleavage buffer) to get the crude peptide acid.

### Example 2. Synthesis of M6009 (SEQ ID NO: 119)

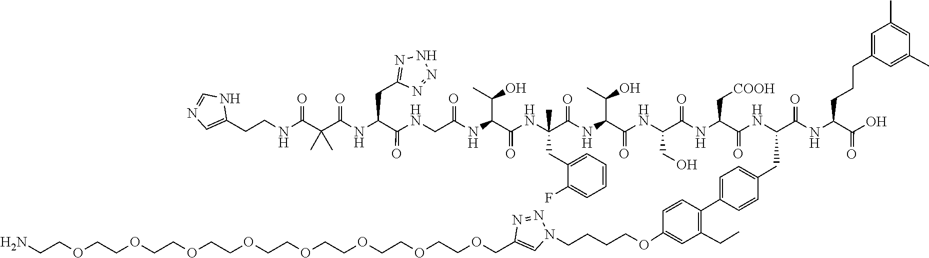

TABLE 4
Properties of M6009

| | |
|---|---|
| Free base MF: C₉₄H₁₃₅FN₂₀O₂₆, | MW: 1980.23 |
| TFA salt MF: C₉₄H₁₃₅FN₂₀O₂₆ *2TFA; | MW: 1980.23 |
| 2208.27 | |
| Appearance | White solid at −20° C. |
| Solubility | Soluble in water >70 mg/ml (>30 mM) |
| Purity | >98% by UPLC and LC-MS |
| Special liability | stable at pH <3 or pH >11, and unstable at PH <1 |
| Storage recommendation | Store solid at −20° C. |
| Absorbance | at 220 nm |

M6009 was synthesized via solid phase synthesis on CTC resin with 5 unnatured amino acids and 5 natured amino acid building blocks, including Fmoc-AA2 (Boc-Linker)-OH and AA9. The detailed reaction conditions on each step from CTC resin (15 mmol, 21.43 g) are shown in Table 5.

TABLE 5
Reagents and Reaction Conditions

| | Reagent | | | Base | | Coupling reagent | | | |
|---|---|---|---|---|---|---|---|---|---|
| Step | | | Amt/Mol | | Mol | | Mol | Temp | Time, |
| # | AA | Source | eq. | Name | eq. | Name | eq. | ° C. | min |
| 1 | Fmoc-AA1- | WuXi | 6675 mg/ | DIEA | 2 | N/A | N/A | 22-28 | 120 |
| | OH | | 1.0 eq | | | | | | |
| 2 | Fmoc- | WuXi | 25050 mg/ | N/A | N/A | DIC/Oxyma | 1.5/1.5 | 22-28 | 120 |
| | AA2(Boc- | | 1.5 eq | | | | | | |
| | Linker)-OH | | | | | | | | |
| 3 | Fmoc- | GL | 30900 mg/ | N/A | N/A | DIC/Oxyma | 5.0/5.0 | 22-28 | 40 |
| | Asp(OtBu)- | Biochem | 5.0 eq | | | | | | |
| | OH | | | | | | | | |
| 4 | Fmoc- | GL | 42750 mg/ | N/A | N/A | DIC/Oxyma | 5.0/5.0 | 22-28 | 40 |
| | Ser(Trt)-OH | Biochem | 5.0 eq | | | | | | |
| 5 | Fmoc-Thr-OH | GL | 25650 mg/ | N/A | N/A | DIC/Oxyma | 5.0/5.0 | 22-28 | 40 |
| | | Biochem | 5.0 eq | | | | | | |
| 6 | Fmoc-α-Me- | GL | 18900 mg/ | N/A | N/A | DIC/Oxyma | 3.0/3.0 | 22-28 | 40 |
| | Phe(2-F)-OH | Biochem | 3.0 eq | | | | | | |
| 7 | Fmoc-Thr-OH | GL | 25650 mg/ | N/A | N/A | DIC/Oxyma | 5.0/5.0 | 22-28 | 16 |
| | | Biochem | 5.0 eq | | | | | | hrs |
| 8 | Fmoc-Gly-OH | GL | 22350 mg/ | N/A | N/A | DIC/Oxyma | 5.0/5.0 | 22-28 | 40 |
| | | Biochem | 5.0 eq | | | | | | |
| 9 | Fmoc-AA9- | WuXi | 23850 mg/ | N/A | N/A | DIC/Oxyma | 3.0/3.0 | 22-28 | 40 |
| | OH | | 3.0 eq | | | | | | |
| 10 | Fmoc-AA10- | WuXi | 17550 mg/ | N/A | N/A | DIC/Oxyma | 2.5/2.5 | 22-28 | 40 |
| | OH | | 2.5 eq | | | | | | |

| | | |
|---|---|---|
| 11 | | Added 3% NH₂NH₂ in DMF (200.0 mL) to the resin and |
| | | agitated with N₂ for 30 mins. |
| 12 | Cleavage and | Using 2.5% H₂O, 7.5% 3-MPA, 2.5% TIS, and 87.5% TFA. |
| | deprotection | |
| | of Boc, Trt, | |
| | tBu | |
| 13 | HPLC | Obtained 12.8 g of M6009 in overall 38.7%. |
| | purification | |

### 2.1 Synthesis, Cleavage, and Impurity Analysis of M6009

### Step 1—1^{st} Resin Preparation

To the CTC resin (15.0 mmol, 1.00 eq., Sub 0.7 mmol/g) was added a solution of Fmoc-AA1-OH (6675 mg, 15.0 mmol, 1.0 eq.), DIEA (2.0 eq.) in DCM (150.0 mL) and the mixture was agitated with N₂ for 2 hours. Then 22.0 mL of MeOH (1 mL/g Resin) was added to the resin for capping the residue reactive site on the CTC resin. Then the mixture was agitated with N₂ for 30 mins and was filtered to get the resin.

### Step 2-1st Deprotection of N-Fmoc

20% Piperidine in DMF (200.0 mL) was added to the resin and the mixture was agitated with N₂ for 20 mins. The resin was washed with DMF (200.0 mL) 5 times and filtered. The deprotection was monitored by ninhydrin color reaction.

### Step 3-2^{nd} Coupling Reaction

A solution of Fmoc-AA2 (Boc-Linker)-OH (25050 mg, 22.5 mmol, 1.5 eq.), DIC (2850 mg, 1.5 eq.), and Oxyma (3225 mg, 1.5 eq.) in DMF (100.0 mL) was added to the resin and the mixture was agitated with N₂ for 2 hours. The coupling reaction was monitored by ninhydrin color reaction.

### Step 4-2^{nd} Deprotection

20% Piperidine in DMF (200.0 mL) was added and the resin was agitated with N₂ for another 20 mins. The resin was washed with DMF (200.0 mL) 5 times and filtered. The deprotection was monitored by ninhydrin color reaction.

### Step 5-3^{rd} Coupling

A solution of Fmoc-Asp (OtBu)-OH (30900 mg, 75.0 mmol, 5.0 eq), DIC (9450 mg, 5.0 eq), and Oxyma (10650 mg, 5.0 eq) in DMF (100.0 mL) was added to the resin and the mixture was agitated with N₂ for 40 mins. The resin was washed with DMF (200.0 mL) 5 times and filtered to get the resin. The coupling reaction was monitored by ninhydrin color reaction.

Step 6—4^{th}-6^{th} Coupling/Deprotection

Above steps were repeated with following amino acids stepwise: Fmoc-Ser(Trt)-OH (42750 mg, 5.0 eq), Fmoc-Thr-OH (25650 mg, 5.0 eq), Fmoc-a-Me-Phe(2-F)-OH (18900 mg, 3.0 eq) using DIC/Oxyma as the coupling regents. Deprotections of N-Fmoc were carried out following each coupling.

Step 7—7^{th} Coupling with AA7 and De-Fmoc Reactions

A solution of Fmoc-Thr-OH (25650 mg, 5.0 eq), DIC (9450 mg, 5.0 eq), and Oxyma (10650 mg, 5.0 eq) in DMF (100.0 mL) was added to the resin and the mixture was agitated with N₂ for 16 hours. The coupling reaction was monitored by ninhydrin color reaction. Deprotection of N-Fmoc was carried out as described above.

Step 8—8^{th} to 9^{th} Coupling and De-Fmoc Reactions

The above step 5 for coupling reaction was repeated with Fmoc-Gly-OH (22350 mg, 5.0 eq) as AA8 and AA9 (23850 mg, 3.0 eq), respectively, together with DIC/Oxyma as the coupling regents. Step 4 for deprotection of N-Fmoc was repeated.

### Step 9—10^{th} Coupling Reaction

A solution of Fmoc-AA10-OH (17750 mg, 2.5 eq), DIC (4800 mg, 2.5 eq), and Oxyma (5325 mg, 2.5 eq) in DMF (100.0 mL) was added to the resin, which was agitated with N₂ for 40 mins. The resin was washed with DMF (200.0 mL) 5 times and filtered. The coupling reaction was monitored by ninhydrin color reaction.

### Step 10

3% NH₂NH₂ in DMF (200.0 mL) was added to the resin. The resin was agitated with N₂ for 30 mins, washed with DMF (200.0 mL) 5 times and filtered.

### Step 11

The resin was washed with MeOH (200.0 mL) 3 times and dried under vacuum and to get the resin bound M6009 (60.0 g).

### Step 12—Cleavage of M6009 from CTC Resin and Deprotection of all Protecting Groups

To the above resin bound M6009 (60.0 g) was added 600 mL of cleavage cocktail (2.5% H₂O, 7.5% 3-MPA, 2.5% TIS, and 87.5% TFA) and the mixture was stirred with magnetic stirrer at room temperature (26° C.) for 2 hours. The mixture was filtered and the filtrate was concentrated to a volume of 180 mL. Cold isopropyl ether (20V or 3600 mL) was slowly added to the concentrated cleavage solution containing the peptide (M6009) with stirring. The peptide precipitated and the mixture was centrifuged (2 min at 3000 rpm). The precipitate was collected and washed two times with isopropyl ether (2000 mL). The crude peptide M6009 was dried under vacuum for 2 hours to generate 27.0 g as white solid. LC-MS of crude material showed that the sample was 75.7% pure.

In analytical HPLC with Rt=10.448 min @220 nm and mass peaks of [M+2H]²⁺=990.8, [M+3H]³⁺=661, [M+4H]⁴⁺=496.1.

The crude M6009 (27 g) was dissolved in H₂O (480 mL) and ACN (60 mL) and the resulting solution was purified by prep-HPLC: 9 g/180 mL per injection, a total of three injections. The following prep-HPLC conditions were used: mobile phase A was 0.0750% TFA in H₂O; mobile phase B was ACN (Table 6). All fractions (around 98% purity) were collected and then lyophilized to obtain pure M6009-TFA salt (12815 mg, 98.82% purity) as a white solid in 38.700 yield (Table 7). Other impurity peaks were also collected during purification, details are as follows.

TABLE 6
HPLC Conditions

| Instrument | Qing bohua DAC100 | |
|---|---|---|
| Mobile Phase | A: ACN | |
| | B: H₂O (0.075% TFA in H₂O) | |
| | Time | A% |
| Gradient | 0 | 5 |
| | 3 | 25 |
| | 53 | 55 |
| | 53.1 | 95 |
| | 60 | 95 |
| | 60.1 | 5 |
| | 68 | 5 |

| | |
|---|---|
| Column | luna c18 10 um 100 A, length: 25 cm, diameter: 10 cm. |
| Flow Rate | 400 mL/Min |
| Wavelength | 214/254 nm |
| Oven Tem. | 26° C. |

TABLE 7
Collected Fractions

| Compound | Amount (mg) | Purity (%) | Comments |
|---|---|---|---|
| M6009-TFA salt | 12815 | 98.82 | Rt = 10.448 min; 38.7% yield |
| Impurity-1 | 1757 | 57.27 | Rt = 9.74 min. |
| Impurity-2 | 727 | 81.15 | Rt = 10.92 min. |
| Impurity-3 | 2680 | 72.04 | All other impurities |
| AA1-AA2 | 120.8 | 96.27 | purified from 300 mg of Impurity-1. |
| tBu-M6009 | 10.7 | 97.57 | purified from 300 mg of Impurity-2. |

M6009 was mixed with each impurity isolated from HPLC as follows. Each impurity and M6009 were separately dissolved in water at concentration (2 mg/mL). Different impurities were mixed with M6009 (Table 8). The mixtures were evaluated by LCMS/HPLC.

TABLE 8
Mixtures Evaluated by LCMS/HPLC

| Sample | Conc. | Sample-1 | Sample-2 | Sample-3 | Sample-4 | Sample-5 | Sample-6 | Sample-7 |
|---|---|---|---|---|---|---|---|---|
| M6009 | 2 mg/ml | 1 ml | N/A | N/A | N/A | 1.25 ml | 1.0 ml | 1.0 ml |
| Impurity-1 | 2 mg/ml | N/A | 1 ml | N/A | N/A | 0.2 ml | N/A | N/A |
| Impurity-2 | 2 mg/ml | N/A | N/A | 1 ml | N/A | 0.1 ml | N/A | N/A |
| Impurity-3 | 2 mg/ml | N/A | N/A | N/A | 1 ml | N/A | 0.21 ml | 0.105 ml |
| MS cal. (Da) | | 1980.23 | 992.58 | 2036.33 | N/A | 1980.23 | 1980.23 | 1980.23 |
| MS observed | | 990.9 | 993.8 | 1019 | N/A | 990.9 | 990.9 | N/A |
| (Da) | | 661.1 | 497.5 | 679.8 | | 661.1 | 661.1 | |
| | | 496.1 | | | | 496.1 | 496.1 | |
| Purity | | 98.64%, | 57.27% | 81.15% | ~72.04% | 81.39% | 86.1% | 95.11%, |
| RT (min) | | 10.358 | 9.657 | 10.833 | | 10.379 | 10.42 | 10.933 |
| Impurity | | N/A | Impurity-1 | Impurity-2 | Impurity-3 | Impurity-1 | Impurity-3 | Impurity-3 |
| content | | | (57.27%) + | (81.15%) + | (72.04%) + | (9.81%) + | (13.9%) + | (4.89%) + |
| | | | M6009 | M6009 | M6009 | Impurity-2 | M6009 | M6009 |
| | | | | | (27.96%) | (7.68%) + | (86.1%) | (95.11%) |
| | | | | | | M6009 | | |
| | | | | | | (81.39%) | | |

After all other impurities (impurity-3) and M6009 were mixed with a mass ratio of 1.05 to 10, the purity of M6009 was more than 95%. The structure of impurity 3 was not identified.

### 2.2 Analytical Data for M6009

UPLC of M6009 showed that the sample was 98.82% pure. UPLC of M6009 was monitored at 220 nm.

LCMS of M6009 sample showed a main peak with retention time of 1.588 min, which corresponded to the desired product M6009 with the observed mass of [M+H]⁺=1981.4, [M+2H]²⁺=990.9, [M+3H]³⁺=661, [M+4H]⁴+=496.1.

¹H NMR (400 MHz, METHANOL-d4) δ ppm 1.04 (3H, t, J=8.00 Hz), 1.25 (6H, dd, J=8.00, 4.00 Hz), 1.36 (6H, d, J=12.00 Hz), 1.44 (3H, s), 1.68-1.79 (2H, m), 1.81-1.90 (3H, m), 2.16 (2H, s), 2.26 (6H, s), 2.43-2.70 (6H, m), 2.94 (3H, br t, J=8.00 Hz), 3.14-3.19 (2H, m), 3.21-3.29 (2H, m), 3.39-3.73 (33H, m), 3.74-3.78 (2H, m), 3.85-3.92 (1H, m), 3.95-4.12 (6H, m), 4.15-4.22 (1H, m), 4.26-4.32 (1H, m), 4.37-4.50 (3H, m), 4.54 (3H, m), 4.67 (3H, s), 6.75-6.83 (4H, m), 6.85 (2H, d, J=4.00 Hz), 6.91-6.95 (1H, m), 6.91-7.02 (3H, m), 7.05 (1H, d, J=8.00 Hz), 7.17-7.22 (1H, m), 7.25 (2H, d, J=8.00 Hz), 7.35 (1H, s), 7.64-7.70 (1H, m), 7.84 (2H, s), 8.04 (2H, s), 8.49-8.54 (1H, m), 8.78 (1H, d, J=4.00 Hz).

### Example 3. Synthesis of M6324 (SEQ ID NO: 239)

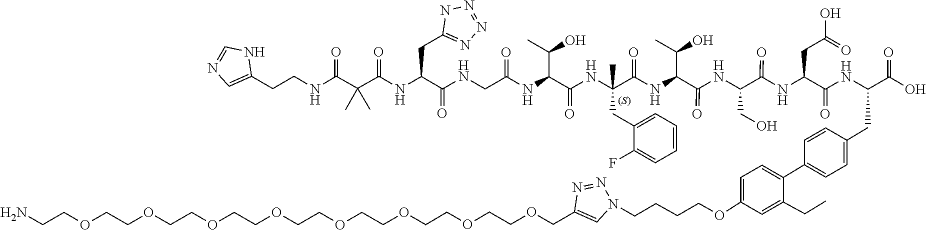

TABLE 9
Properties of M6324

| | |
|---|---|
| Free base MF: C₈₁H₁₁₈FN₁₉O₂₅; | MW: 1775.85 |
| Appearance | White solid at −20° C. |
| Solubility | Soluble in water >70 mg/ml (>30 mM) |
| Purity | >98% by UPLC and LC-MS |
| Storage recommendation | Store solid at −20° C. |

### 3.1 Synthesis, Cleavage, and Impurity Analysis of M6324

M6324 was synthesized according to the following synthetic schemes (SEQ ID NO: 239 disclosed below):

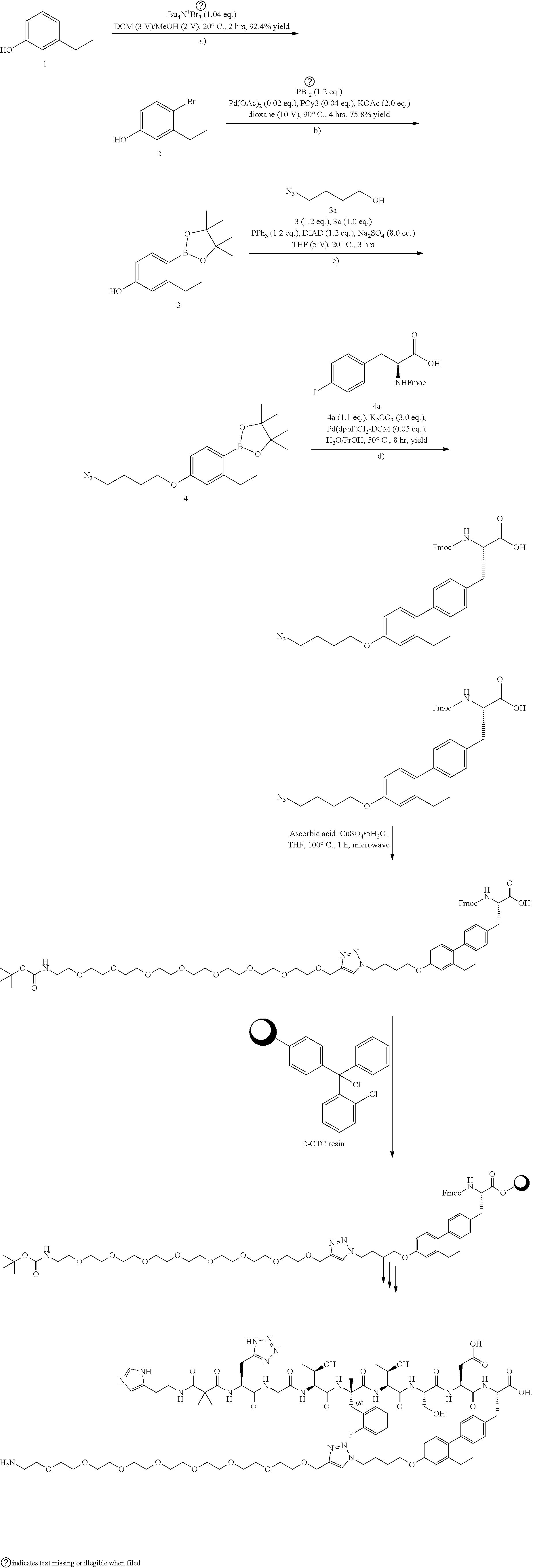

An alternative synthetic route is shown below (SEQ ID NO. 239 disclosed below).

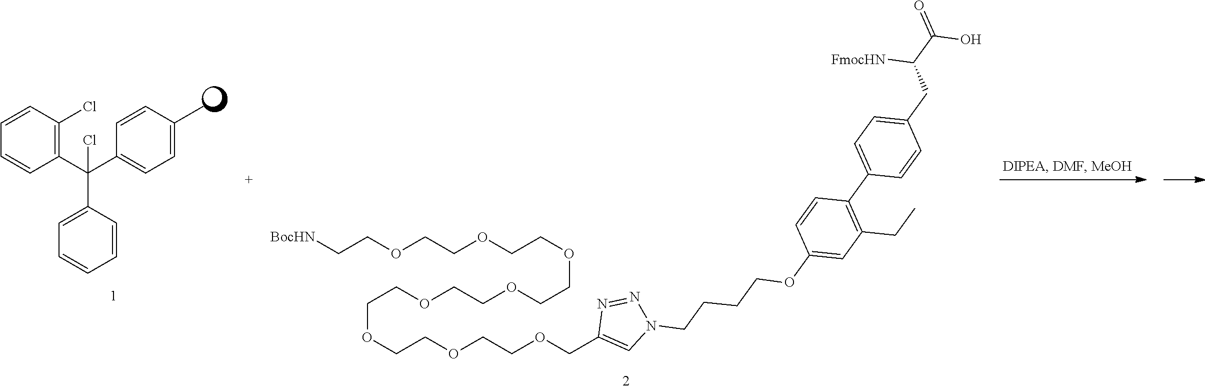

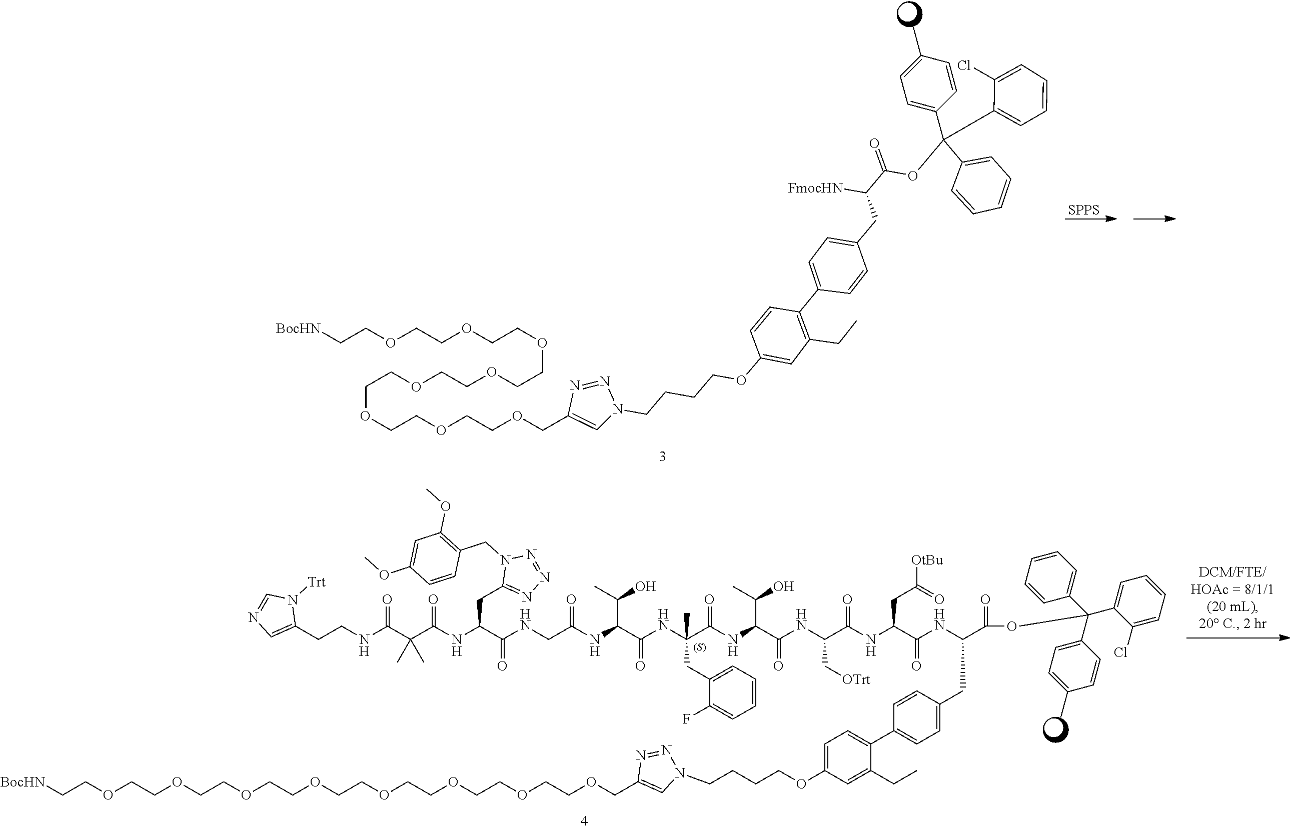

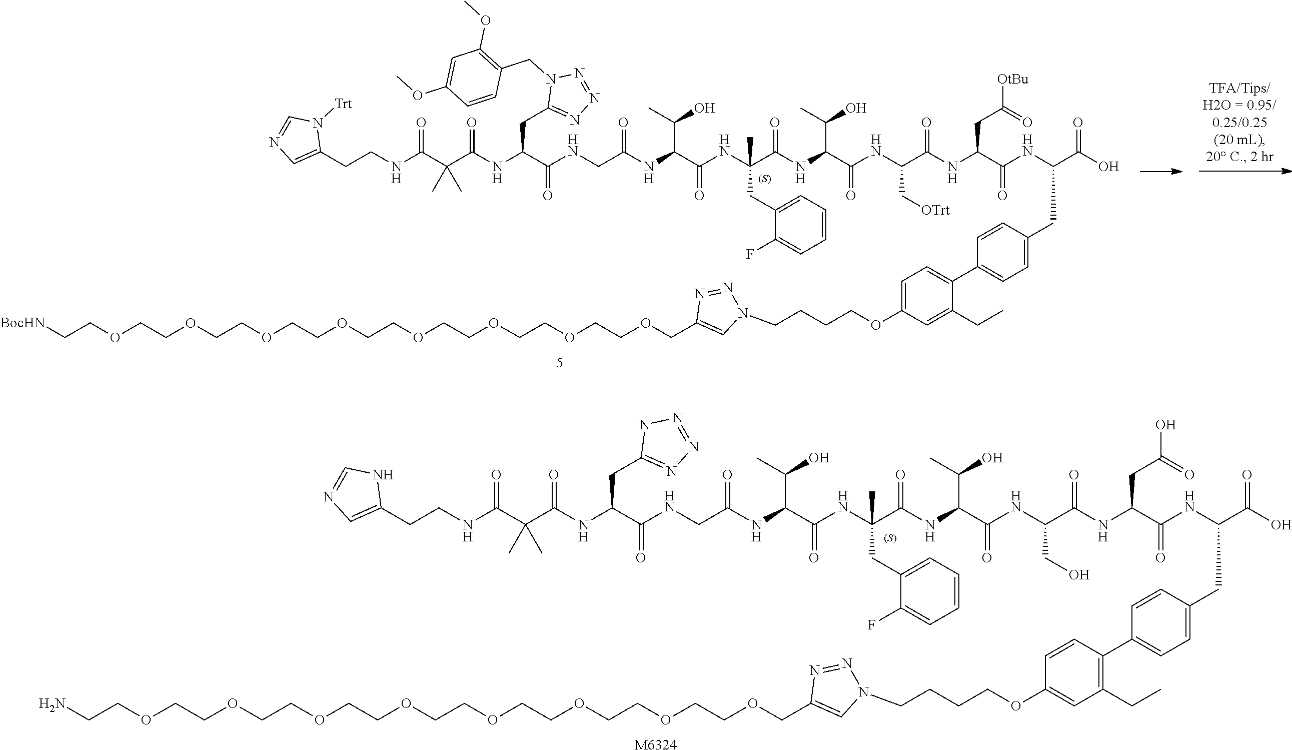

### Step 1: Synthesis of [(2-Chlorophenyl)-diphenyl-methyl](2S)-3-[4-[4-[4-[4-[2-[2-[2-[2-[2-[2-[2-[2-(tertbutoxycarbonylamino)ethoxy]ethoxy]ethoxy]ethoxy]ethoxy]ethoxy]ethoxy]ethoxymethyl]triazol-1-yl]butoxy]-2-ethyl-phenyl]phenyl]-2-(9H-fluoren-9-ylmethoxycarbonylamino)propanoate (3)

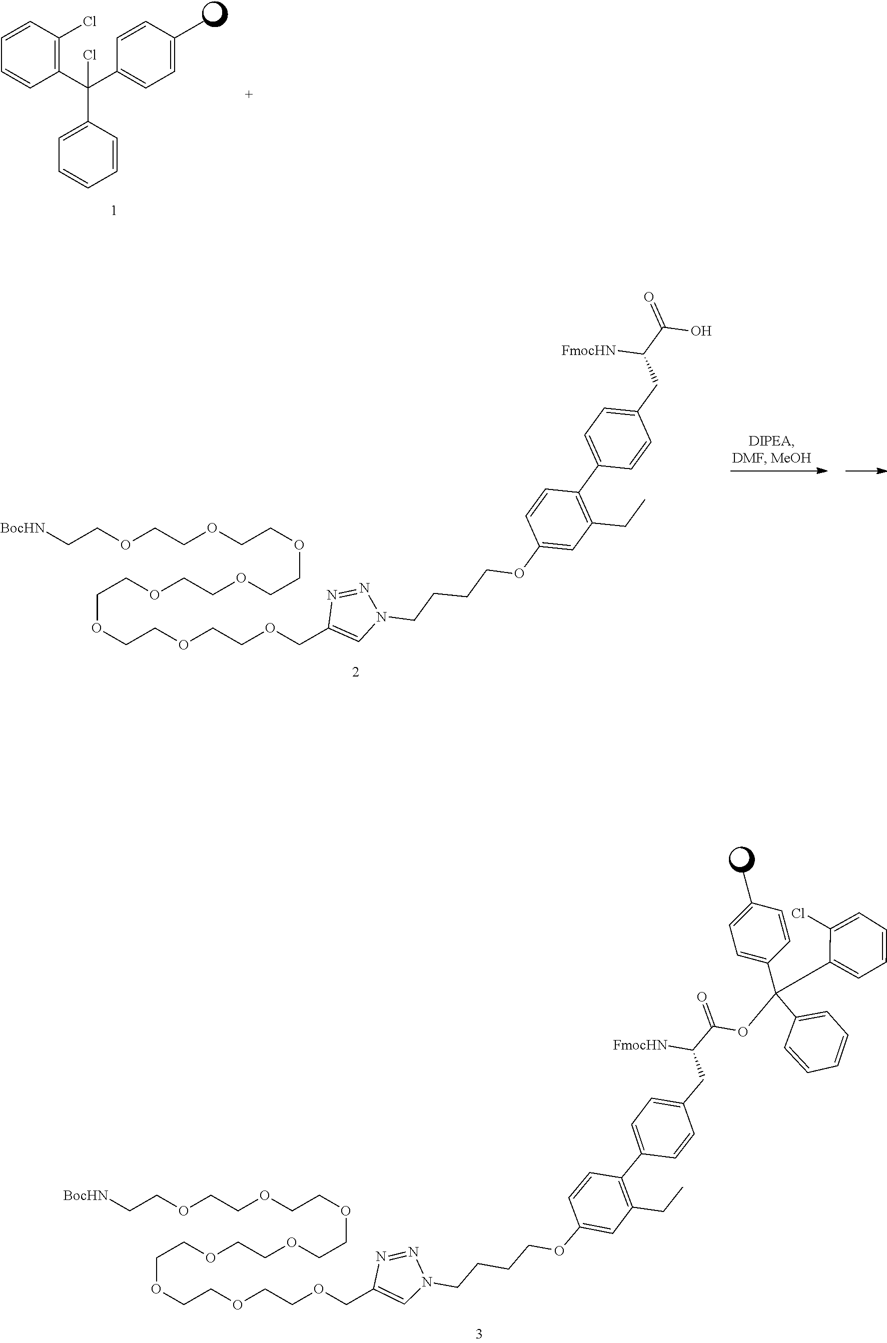

To a mixture of 2-CTC Resin (1) (312.21 mg, 1 mmol, 1 eq.) in DMF (100 mL) were added compound 2 (3.34 g, 3.00 mmol, 1.5 eq.) and DIPEA (1.29 g, 10.00 mmol, 1.74 mL, 10.0 eq.). Then the mixture was shaken at 25° C. for 12 hours. MeOH (30 mL) was added and the mixture was shaken at 25° C. for another 2 hours. LCMS showed that the desired MS was detected. The mixture was filtered and the collected resin was washed with DMF (30 mL×3) and DCM (30 mL×3) to give the crude product on solid phase. Compound 3 (2.78 g, 2.00 mmol, 100.00% yield) was obtained as a yellow solid which was used directly in the next step.

### Step 2: Synthesis of tert-Butyl (3S)-4-[[(]S)-1-[[4-[4-[4-[4-[2-[2-[2-[2-[2-[2-[2-[2-(tert- butoxycarbonylamino)ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[(2-chlorophenyl)-diphenyl-methoxy]-2-oxo-ethyl]amino]-3-[[(2S)-2-[[(2S,3R)-2-[[2-[[(2S,3R)-2-[[2-[[(2S)-3-[ ]-[(2,4-dimethoxyphenyl) methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoate (4)

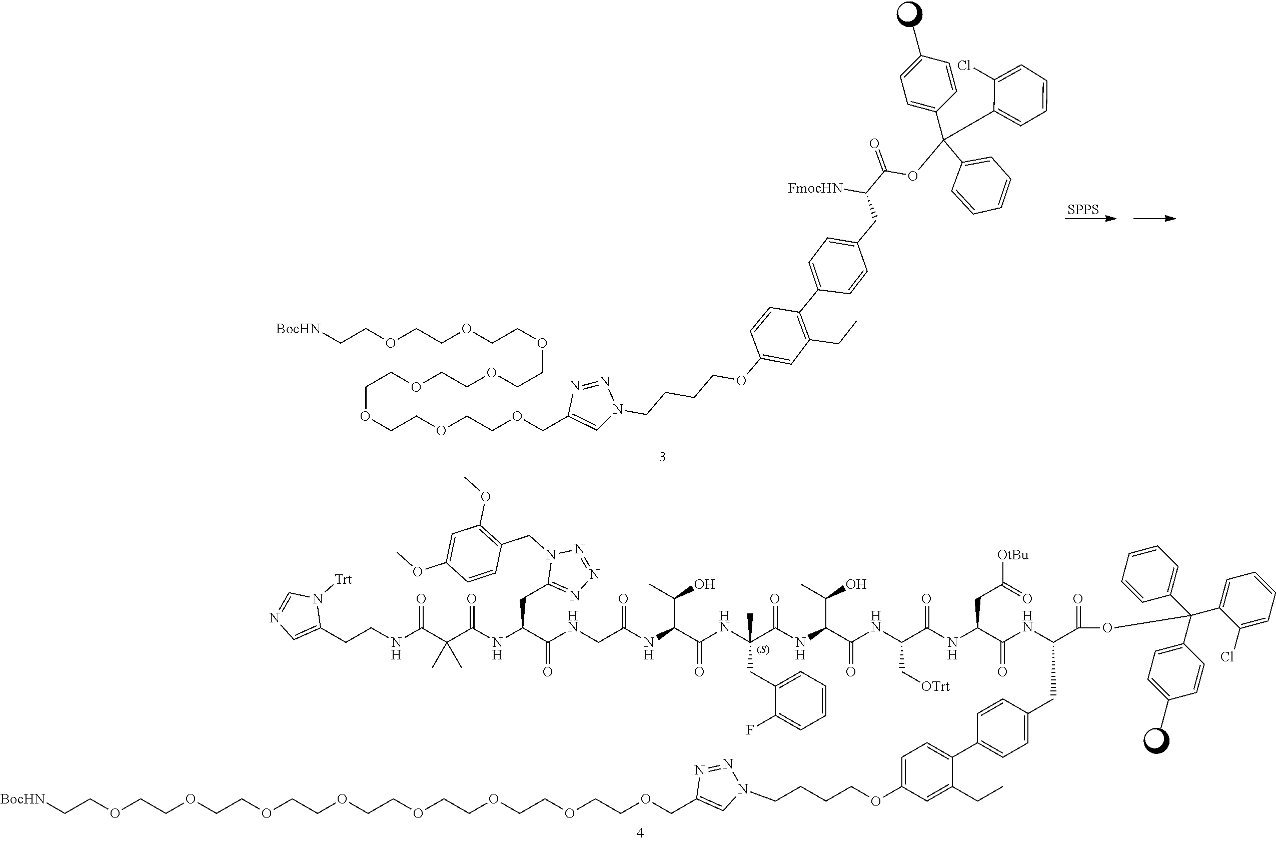

The peptide elongation was performed on a 0.25 mmol scale using liberty Blue Automated Microwave Peptide Synthesizer. Following the standard operation on a peptide synthesizer. Step A: The compound 3 (0.25 mmol, 1 eq.) was swollen with DMF (10 mL) for 300 seconds following the standard operation protocol of the Microwave Peptide Synthesizer. Step B: De-protection was carried out by adding a solution of 20% piperidine/DMF (5 mL) to the resin vessel and agitating with N₂ for 2 min at 90° C. Then the vessel was drained and resin was washed with DMF (3 mL×3) at 20° C. Step C (coupling): A solution of amino acid (2.5 mmol, 5 eq.) in DMF (5 mL), DIC (2 mL), and oxyma (1 mL) were added to the vessel successively and agitated with N₂ for 10 min at 90° C. Steps B and C were carried out for all amino acids. Compound 4 (2.84 g, 998.70 mol, 100.00% yield) was obtained as described in the general procedure of SPPS. The crude product was obtained as a yellow solid which was used directly in the next step.

### Step 3: Synthesis of (2S)-3-[4-[4-[4-[4-[2-[2-[2-[2-[2-[2-[2-(2-Aminoethoxy)ethoxy]ethoxy]ethoxy]ethoxy]ethoxy]ethoxy]ethoxymethyl]triazol-1-yl]butoxy]-2-ethyl-phenyl]phenyl]-2-[[(2S)-4-tert-butoxy-2-[[(2S)-2-[[(2S,3R)-2-[[2-[[(2S,3R)-2-[[2-[[(2S)-3-[1-[(2,4-dimethoxyphenyl) methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoyl]amino]propanoic Acid (5)

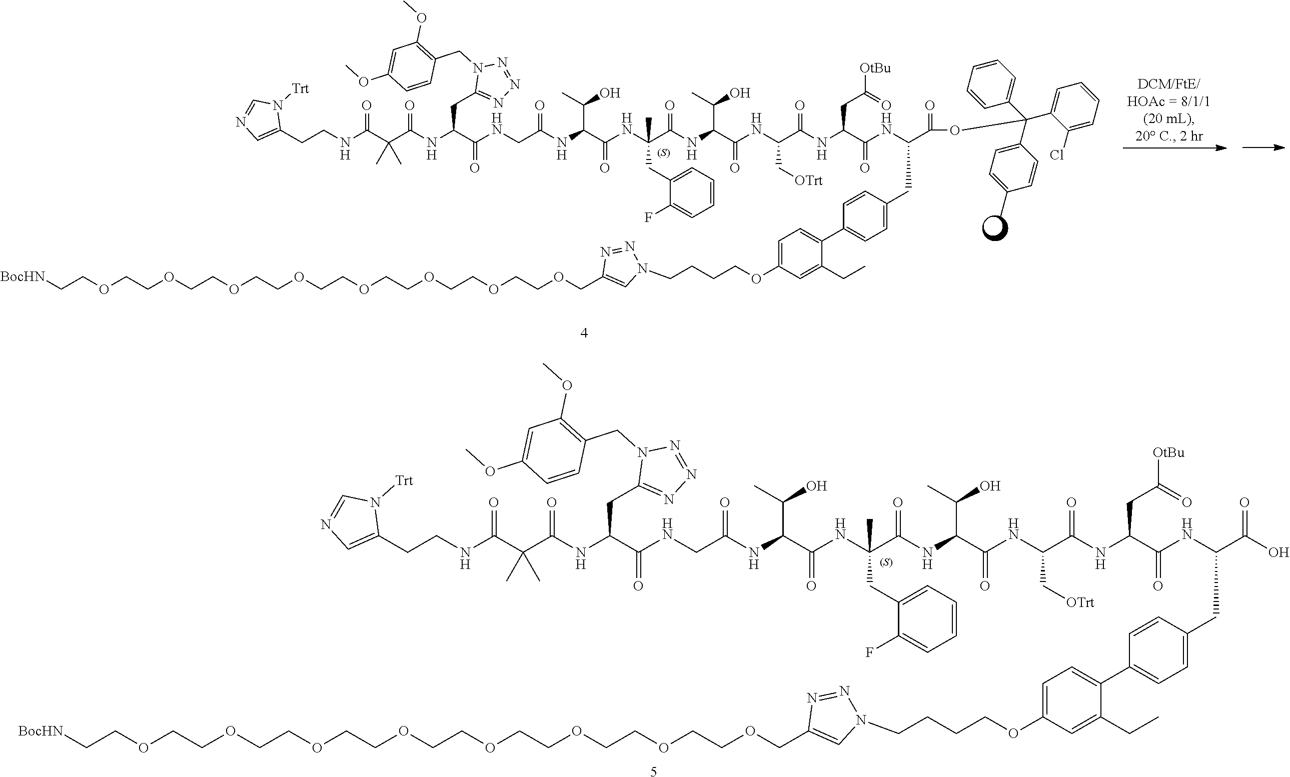

A solution of compound 4 (2.84 g, 998.70 mol, 1 eq.) in DCM (200 mL), TFE (25 mL), and AcOH (25 mL) was stirred at 20° C. for 2 hours. LCMS trace showed that the desired MS was detected. The mixture was filtered and the filtrate was concentrated to give the residue. Compound 5 (2.46 g, 996.83 mol, 100.00% yield) was obtained as a white solid which was used directly in the next step.

LCMS: RT=5.756 min, m/z calcd. for C₁₃₇H₁₇₄FN₁₉O₂₉, 1284.13 [M+2H]²⁺, m/z found 1285.00. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Step 4: Synthesis of (3S)-4-[[(]S)-2-[4-[4-[4-[4-[2-[2-[2-[2-[2-[2-[2-(2-Aminoethoxy)ethoxy]ethoxy]ethoxy]ethoxy]ethoxy]ethoxy]ethoxymethyl]triazol-1-yl]butoxy]-2-ethyl-phenyl]phenyl]-1-carboxy-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(]H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic Acid (SEQ ID NO: 239) (M6324) (SEQ ID NO: 239 disclosed below)

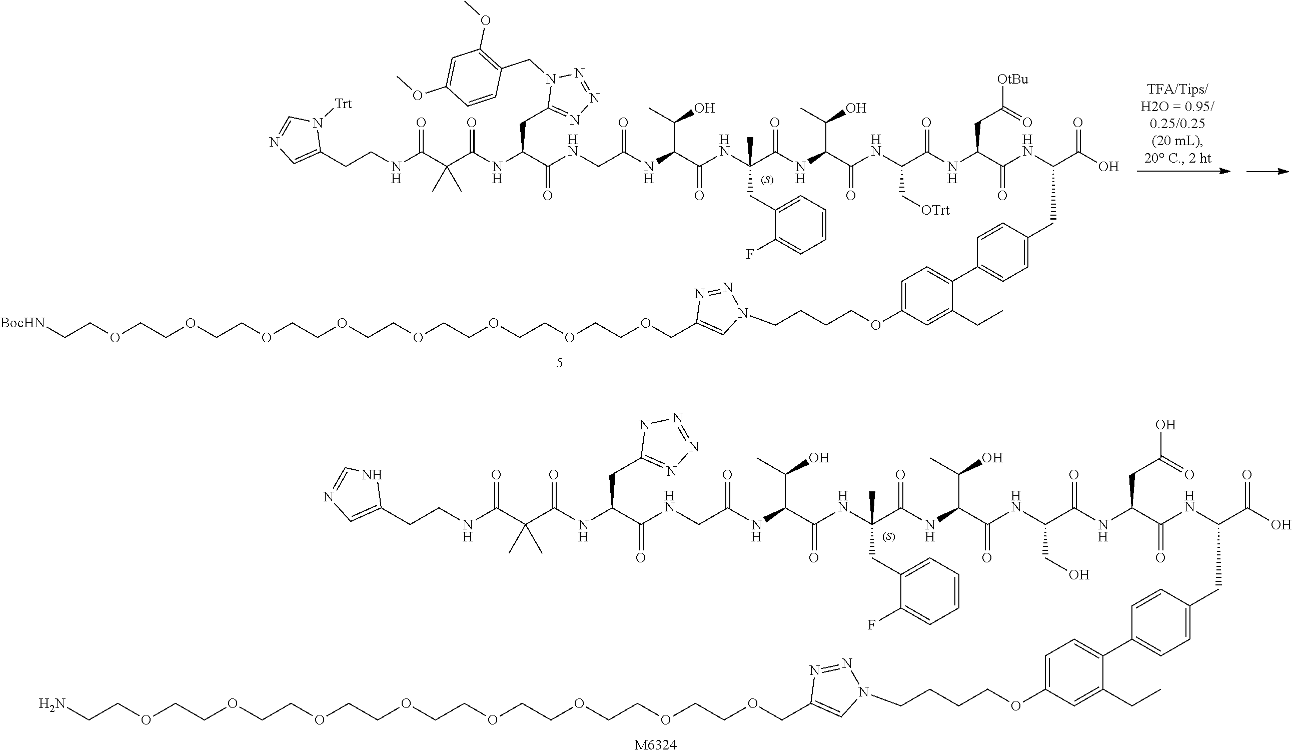

A solution of compound 5 (2.47 g, 1 mmol, 1 eq.) in TFA (38 ml), triisopropylsilane (1 mL), and H₂O (1 mL) was stirred at 20° C. for 2 hours. LCMS trace showed that the desired MS signal was detected. The reaction mixture was triturated with MTBE (200 mL) and the solid was precipitated to give the crude product (1.8 g). 3.2 Purification and Identification of M6324

The crude was purified by prep-HPLC (column: C18 150×30 mm; mobile phase: [water (TFA)-ACN]; gradient: 10%-50% B over 9 min) and prep-HPLC (column: C18 150×40 mm; mobile phase: [water (NH₃H₂O+NH₄HCO₃)-ACN]; gradient: 0%-37% B over 9 min. Compound M6324 (0.5 g, 248.38 mol, 12.42% yield, 98.2% purity) was obtained as a white solid. LCMS: RT=3.047 min, m/z calcd. for C₈₁H₁₂₀FN₁₉O₂₅, 888.93 [M+2H]²⁺, m/z found 889.40. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 mm. Wave length: UV 220 nm, 215 nm, 254 nm. Column temperature: 50° C. HPLC: RT=6.405 min, 99.08% purity. HPLC method A: Column: YMC-Pack ODS-A 150*4.6 mm, Sum; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 ml/min.

### Example 4. Synthesis of Compound 10 (SEQ ID NO: 146) (AA10-AA9-AA8-AA7-AA6-AA5-AA4-AA3-AA2-OH)

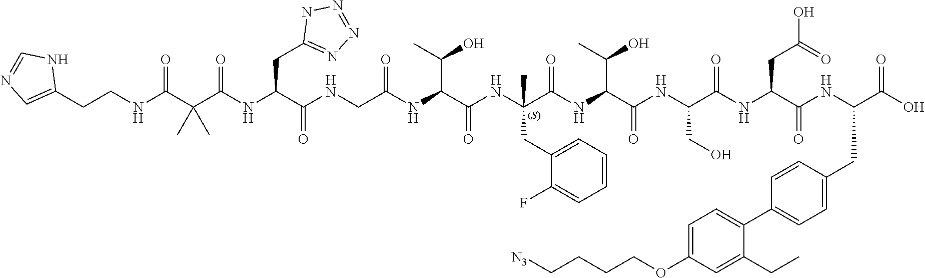

### 4.1 Synthesis, Cleavage, and Impurity Analysis of Compound 10 (SEQ ID NO: 146 Disclosed Below)

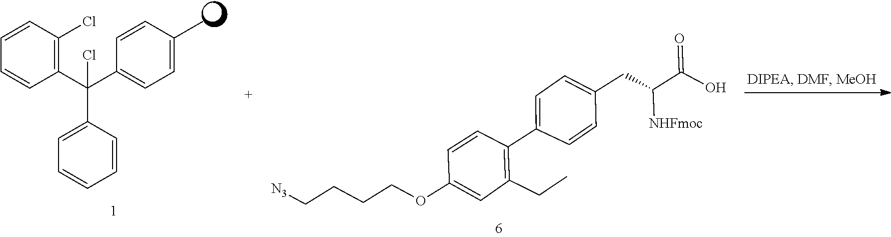

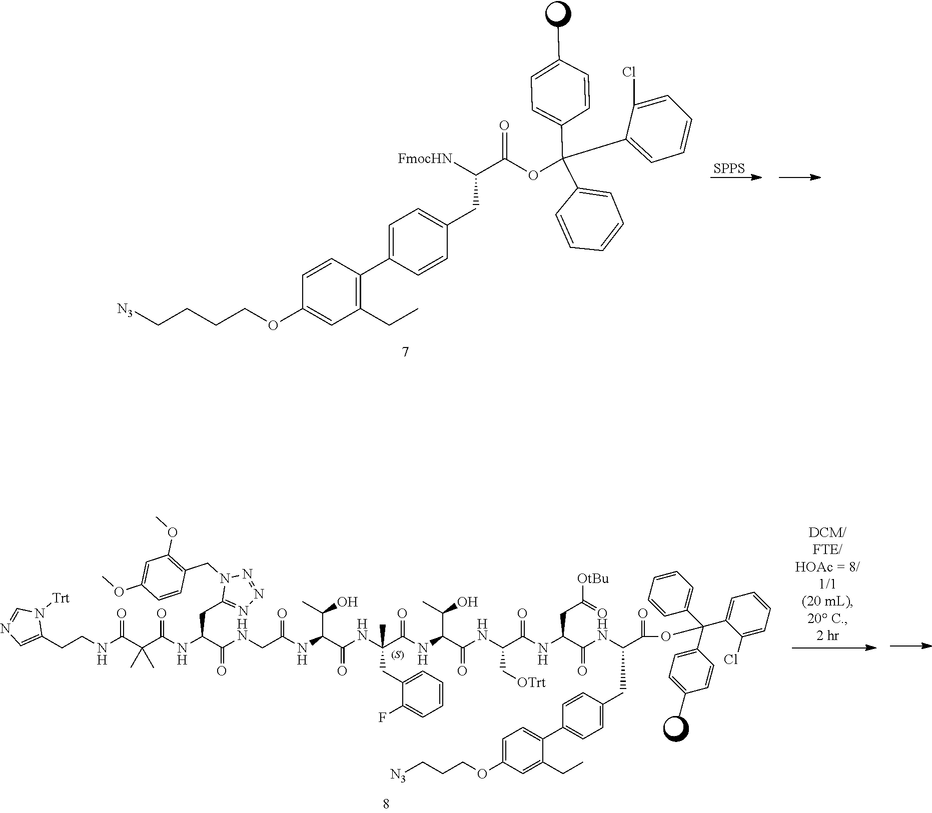

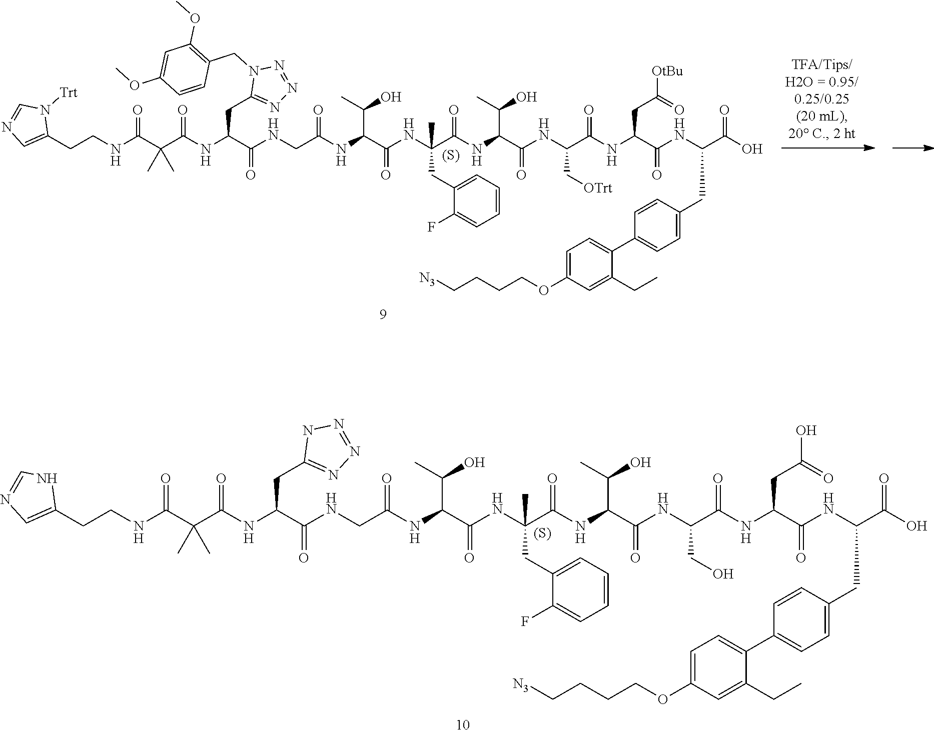

### Step 1: Synthesis of [(2-chlorophenyl)-diphenyl-methyl](2S)-3-[4-[4-(4-azidobutoxy)-2-ethyl-phenyl]phenyl]-2-(9H-fluoren-9-ylmethoxycarbonylamino)propanoate (7)

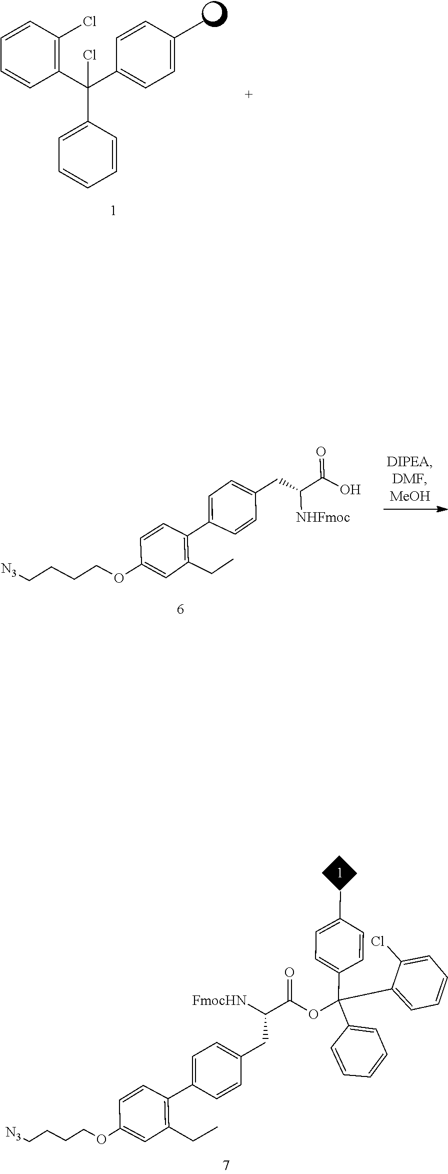

To a solution of resin 1 (312.21 mg, 1 mmol, 1 eq.) in DMF (100 mL) were added compound 6 (1.21 g, 2.00 mmol, 2.0 eq.) and DIPEA (1.29 g, 10.00 mmol, 1.74 mL, 10.0 eq). Then the mixture was shaken at 25° C. for 12 hours. MeOH (30 mL) was added and the mixture was shaken at 25° C. for another 2 hours. LCMS trace showed that the desired MS was detected. The mixture was filtered and the collected resin was washed with DMF (30 mL×3) and DCM (30 mL×3) to give the crude product on solid phase. Compound 7 (880 mg, 999.50 mol, 99.95% yield) was obtained as a yellow solid which was used directly in the next step.

### Step 2: Synthesis of tert-Butyl (3S)-4-[[(]S)-1-[[4-[4-(4-azidobutoxy)-2-ethyl-phenyl]phenyl]methyl]-2-[(2-chlorophenyl)-diphenyl-methoxy]-2-oxo-ethyl]amino]-3-[[(2S)-2-[[(2S,3R)-2-[[2-[[(2S,3R)-2-[[2-[[(2S)-3-[ ]-[(2,4-dimethoxyphenyl)methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoate (8)

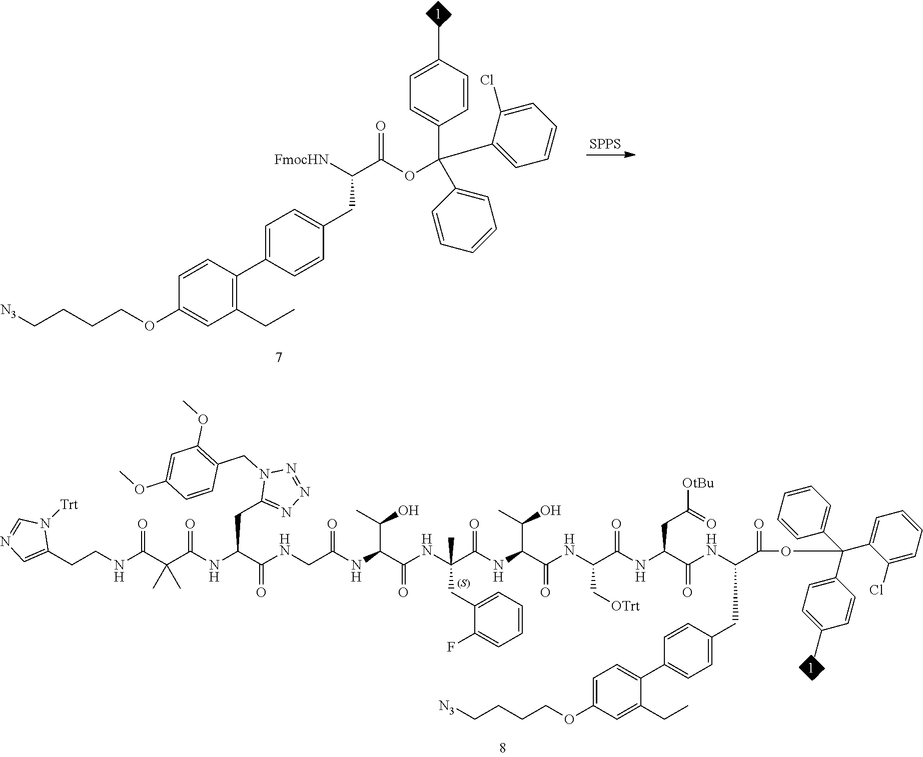

The peptide elongation was performed on a 0.25 mmol scale using liberty Blue Automated Microwave Peptide Synthesizer. Following the standard operation on a peptide synthesizer as follows. Step A: the Rink Amide MBHA Resin (0.5 mmol, 1 eq.) was swollen with DMF (10 mL) for 300 seconds following the standard operation protocol of the peptide synthesizer. Step B (de-protection): A solution of 20% piperidine/DMF (5 mL) was added to the resin vessel and the mixture was agitated with N₂ for 2 min at 90° C. The vessel was then drained and washed with DMF (3 mL×3) at 20° C. Step C (coupling): a solution of amino acid (2.5 mmol, 5 eq.) in DMF (5 mL), DIC (2 mL), and oxyma (1 mL) were added to the vessel successively and the mixture was agitated with N₂ for 10 min at 90° C. Steps B and C were repeated for all amino acids. Compound 8 (2.34 g, 1.00 mmol, 100.00% yield) was prepared as described in the general procedure of SPPS. The crude product was obtained as a yellow solid which was used directly in the next step.

### Step 3: Synthesis of (2S)-3-[4-[4-(4-Azidobutoxy)-2-ethyl-phenyl]phenyl]-2-[[(2S)-4-tert-butoxy-2-[[(2S)-2-[[(2S,3R)-2-[[2-[[(2S,3R)-2-[[2-[[(2S)-3-[1-[(2,4-dimethoxyphenyl)methyl]tetrazol-S-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoyl]amino]propanoic Acid (9)

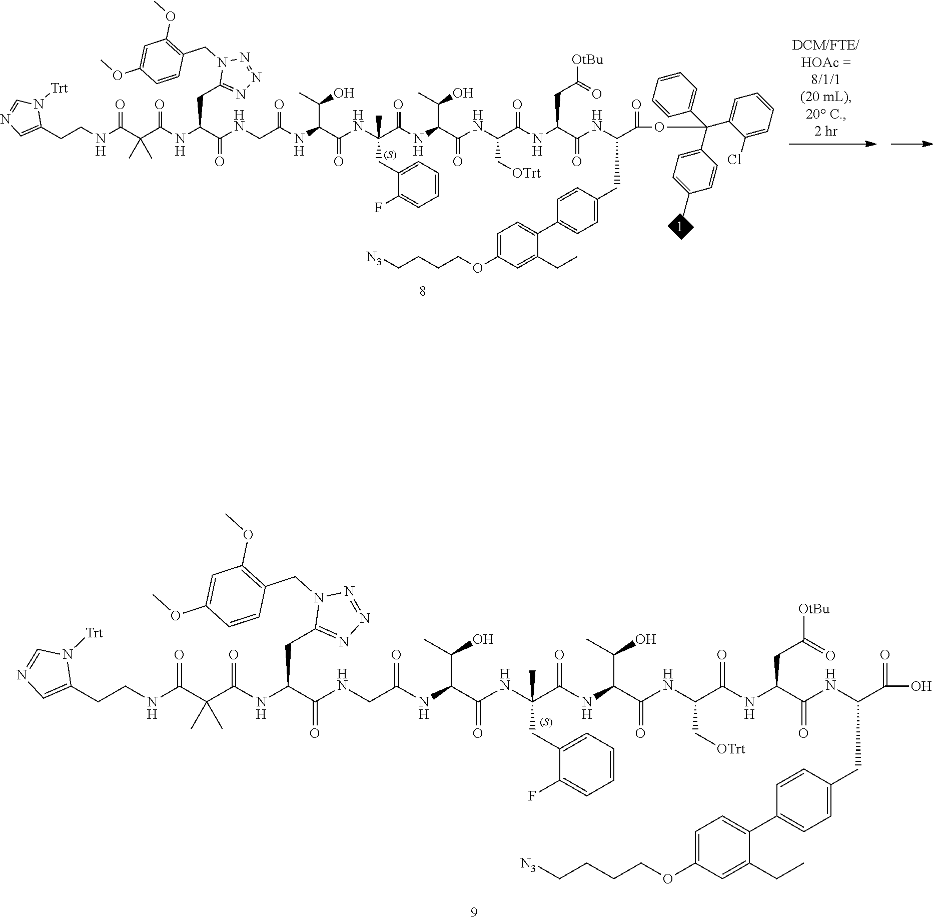

A solution of compound 8 (2.34 g, 1 mmol, 1 eq.) in DCM (100 mL), TFE (12.5 mL), and AcOH (12.5 mL) was stirred at 20° C. for 2 hours. LCMS trace showed that the desired MS was detected. The mixture was filtered and the filtrate was concentrated to give the residue. Compound 9 5 (2.06 g, 999.84 mol, 100.00% yield) was obtained as a white solid which was used directly in the next step.

LCMS RT=3.411 min, m/z calcd. for C₆₂H₈₂FN₁₈O₁₇, 1369.60 [M-2Trt-tBu-DMB-Trt Resin+6H]⁺, m/z found 1369.80. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Step 4: Synthesis of (3S)-4-[[(1S)-2-[4-[4-(4-Azidobutoxy)-2-ethyl-phenyl]phenyl]-1-carboxy-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(1H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic Acid (SEQ ID NO: 146) (10) (SEQ ID NO: 146 disclosed below)

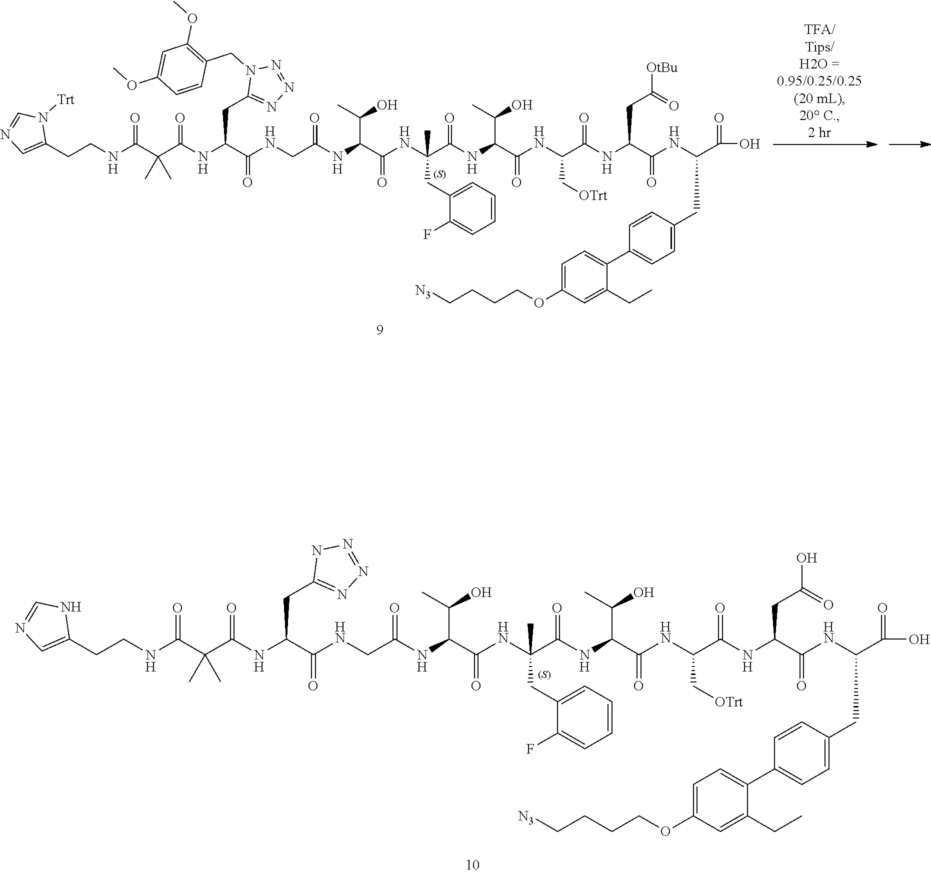

A solution of compound 9 (2.06 g, 1.0 mmol, 1 eq.) in TFA (19 mL), triisopropylsilane (0.5 mL), and H₂O (0.5 mL) was stirred at 20° C. for 2 hours. LCMS trace showed that the desired MS was detected. The reaction mixture was triturated with MTBE (200 mL) and the solid was precipitated to give the crude product (0.4 g). The crude was purified by prep-HPLC (column: C18 150×30 mm; mobile phase: [water (TFA)-ACN]; gradient: 23%-63% B over 9 min) and prep-HPLC (column: C18 150×40 mm; mobile phase: [water (NH₃*H₂O+NH₄HCO₃)-ACN]; gradient: 0%-38% B over 9 min) again. Compound 10 (90 mg, 63.68 mol, 6.37% yield, 96.90% purity) was obtained as a white solid. 4.2 Purification and Identification of Compound 10

LCMS RT=3.973 min, m/z calcd. for C₆₂H₈₂FN₁₈O₁₇, 1368.60 [M+H]⁺, m/z found 1369.90. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

HPLC AC RT=8.204 min, 96.90% purity. HPLC method A: Column: YMC-Pack ODS-A 150*4.6 mm, Sum; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 ml/min.

### Example 5. Synthesis of Compound 15(SEQ ID NO: 241) (AA10-AA9-AA8-AA7-AA6-AA5-AA4-AA3(Q-L)AA2-OH)

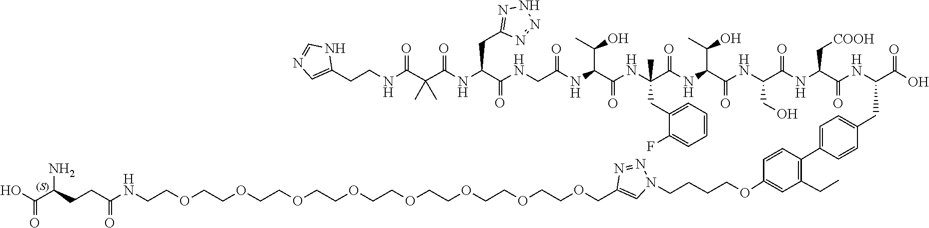

### 5.1 Synthesis, Cleavage and Impurity Analysis of Compound 15 (SEQ ID NOS 239, 337, 338, and 241 Disclosed Below, Respectively in Order of Appearance)

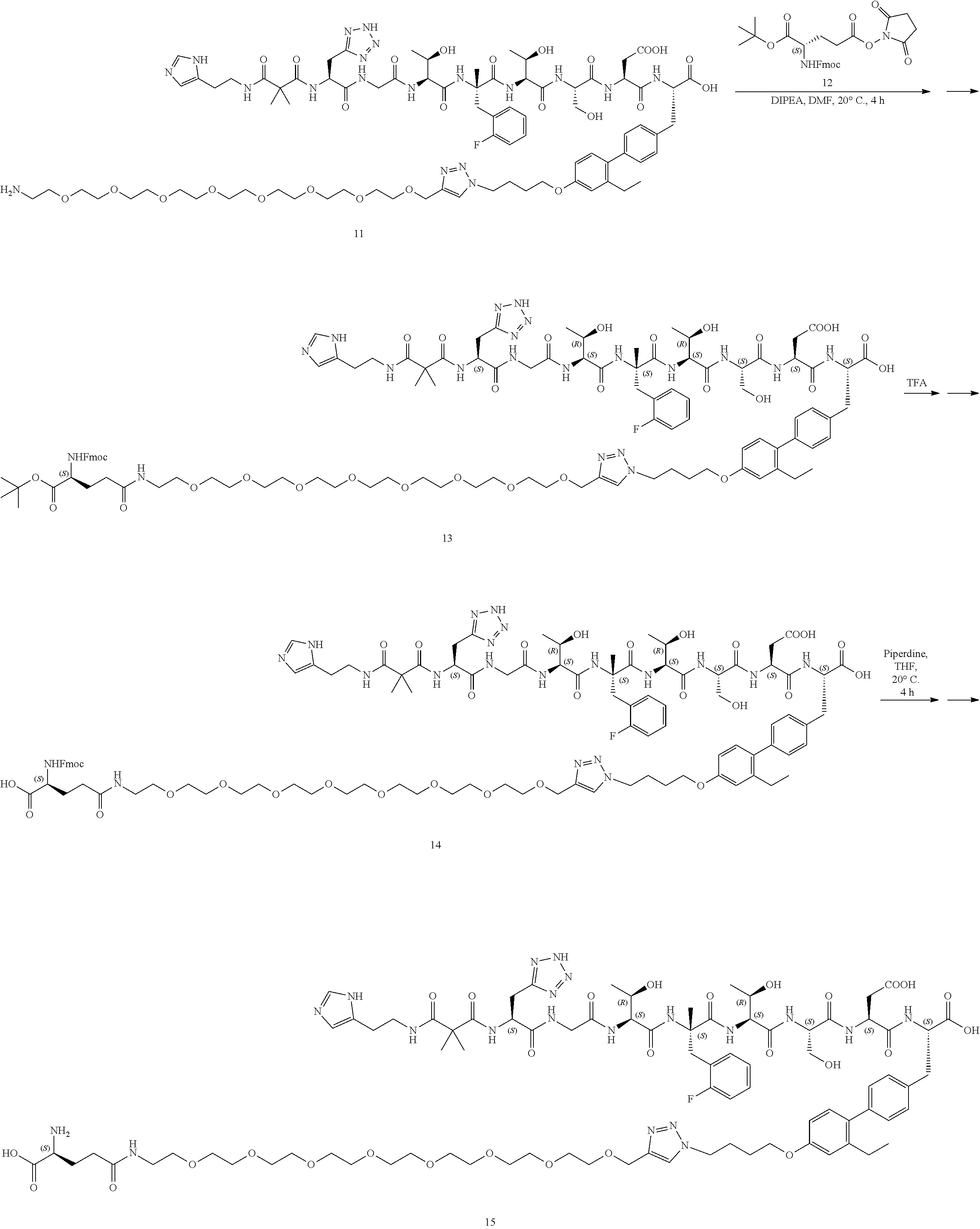

### Step 1: Synthesis of (3S)-4-[[(]S)-2-[4-[4-[4-[4-[2-[2-[2-[2-[2-[2-[2-[2-[[(4S)-5- tert-Butoxy-4-(9H-fluoren-9-ylmethoxycarbonylamino)-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]-1-carboxy-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic Acid (13) (SEQ ID NO: 337) (SEQ ID NOS 239 and 337 Disclosed Below, Respectively, in Order of Appearance)

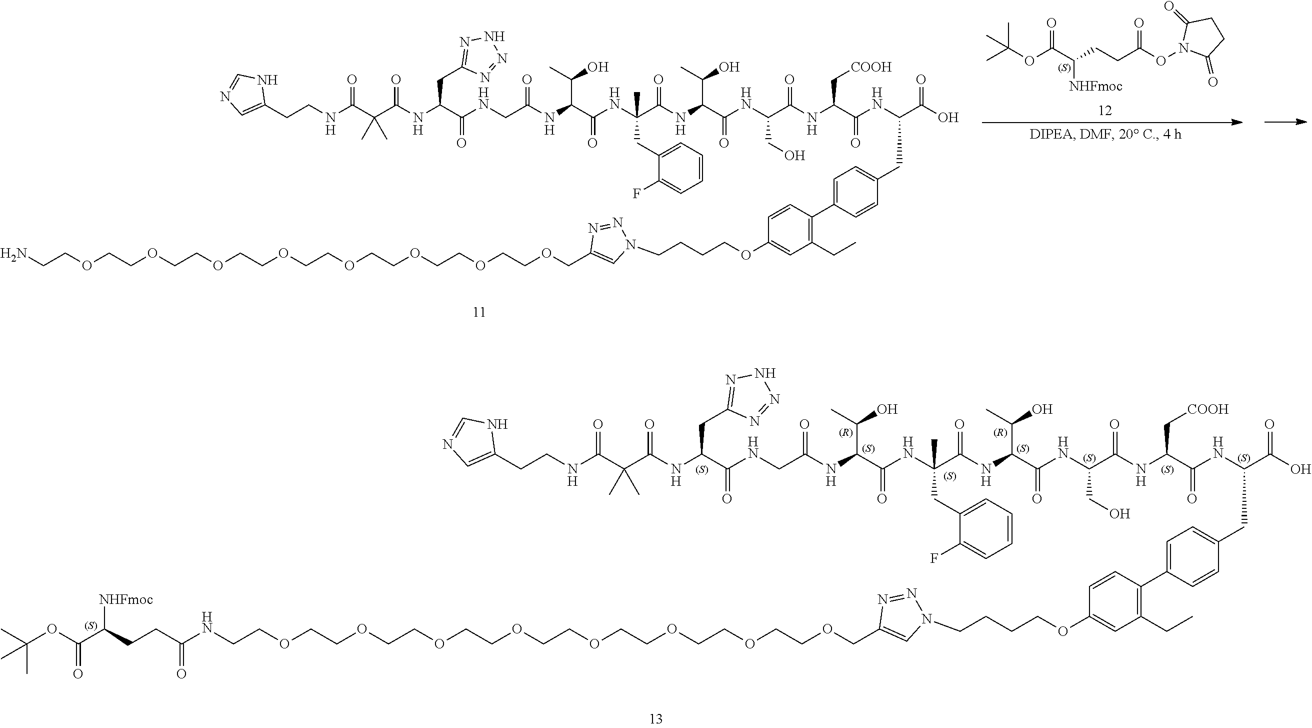

To a solution of compound 11 (200 mg, 112.55 mol, 1 eq) in DMF (3 mL) were added compound 12 (64.70 mg, 123.81 mol, 1.1 eq.) and DIPEA (29.09 mg, 225.11 mol, 39.21 L, 2.0 eq.). Then the solution was stirred at 20° C. for 12 hours. LCMS trace showed that the desired MS was detected. The solvent was removed under reduced pressure to give the crude product. Compound 13 (245 mg, 112.16 mol, 99.65% yield) was obtained as a yellow oil which was used directly in the next step.

LCMS (ESI): RT=4.416 min, m/z calcd. for: C₁₀₅H₁₄₅FN₂₀O₃₀, 1092.52 [M+2H]²⁺; m/z found 1093.20 [M+2H]²⁺, 729.20 [M+3H]³⁺; Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Step 2: Synthesis of (2S)-5-[2-[2-[2-[2-[2-[2-[2-[2-[[ ]-[4-[4-[4-[(2S)-2-Carboxy-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]propanoyl]amino]ethyl]phenyl]-3-ethyl-phenoxy]butyl]triazol-4-yl]methoxy]ethoxy]ethoxy]ethoxy]ethoxy]ethoxy]ethoxy]ethoxy]ethylamino]-2-(9H-fluoren-9-ylmethoxycarbonylamino)-5-oxo-pentanoic Acid (14) (SEQ ID NO: 338) (SEQ ID NOS 337 and 338, Disclosed Below Respectively, in Order of Appearance)

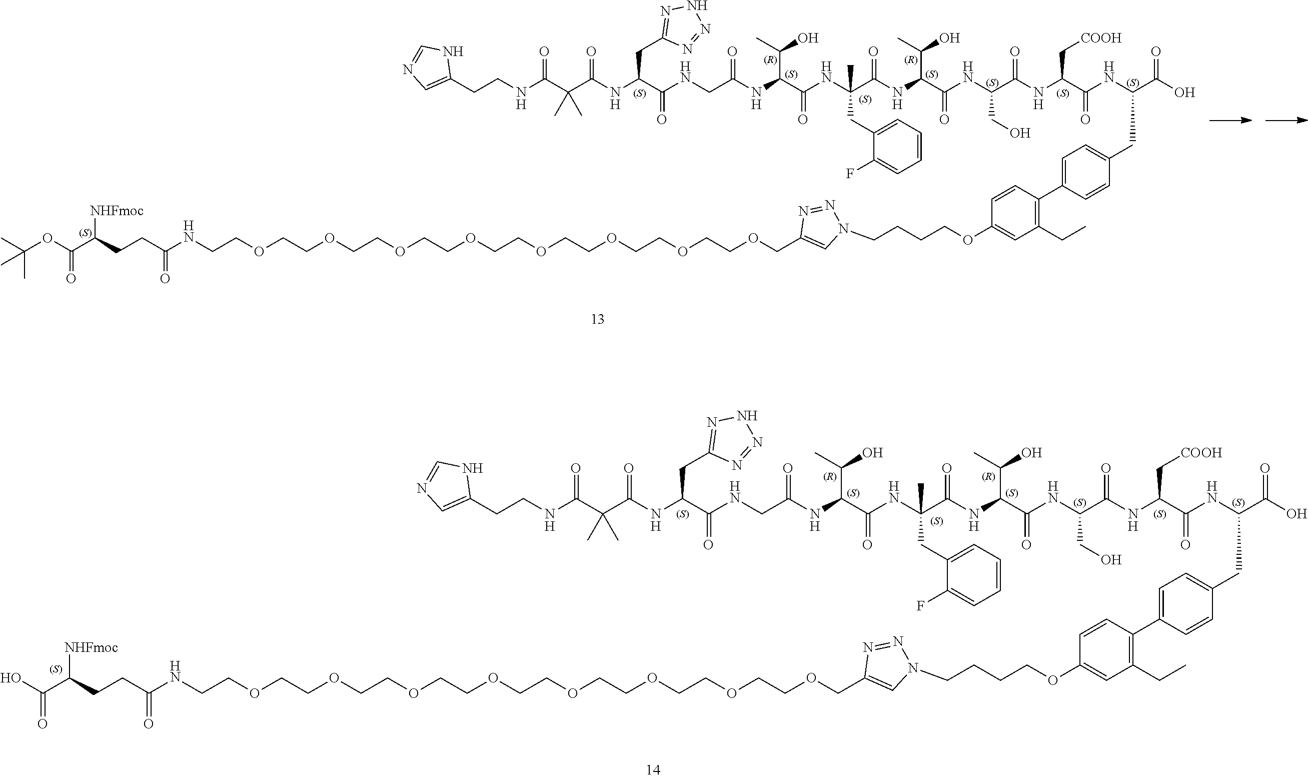

A solution of compound 13 (245 mg, 112.16 mol, 1 eq.) in DCM (3 mL) and TFA (3 mL) was stirred at 20° C. for 12 hours. LCMS trace showed that the desired MS was detected. The solvent was removed under reduced pressure. Compound 14 (238 mg, 111.83 mol, 99.70% yield) was obtained as a yellow oil which was used directly in the next step.

LCMS (ESI): RT=3.959 min, m/z calcd. for C₁₀₁H₁₃₇FN₂₀O₃₀, 1064.48 [M+2H]²⁺; m/z found 1065.20 [M+2H]²⁺. Mobile Phase: 1.5 mL/4 L TFA in water (solvent A) and 0.75 mL/4 L TFA in acetonitrile (solvent B) using the elution gradient 10%-80% (solvent B) over 0.7 minutes and holding at 80% for 0.4 minutes at a flow rate of 1.5 mL/min. Column: Agilent Pursult 5 C18 20*2.0 mm. Wavelength: UV 220 nm and 254 nm. Column temperature: 50° C. MS ionization: ESI 50° C.

### Step 3: Synthesis of (2S)-2-Amino-5-[2-[2-[2-[2-[2-[2-[2-[2-[[ ]-[4-[4-[4-[(2S)-2-carboxy- 2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]propanoyl]amino]ethyl]phenyl]-3-ethyl-phenoxy]butyl]triazol-4-yl]methoxy]ethoxy]ethoxy]ethoxy]ethoxy]ethoxy]ethoxy]ethoxy]ethylamino]-5-oxo-pentanoic Acid (15) (SEQ ID NO: 241) (SEQ ID NOS 338 and 241 Disclosed Below, Respectively, in Order of Appearance)

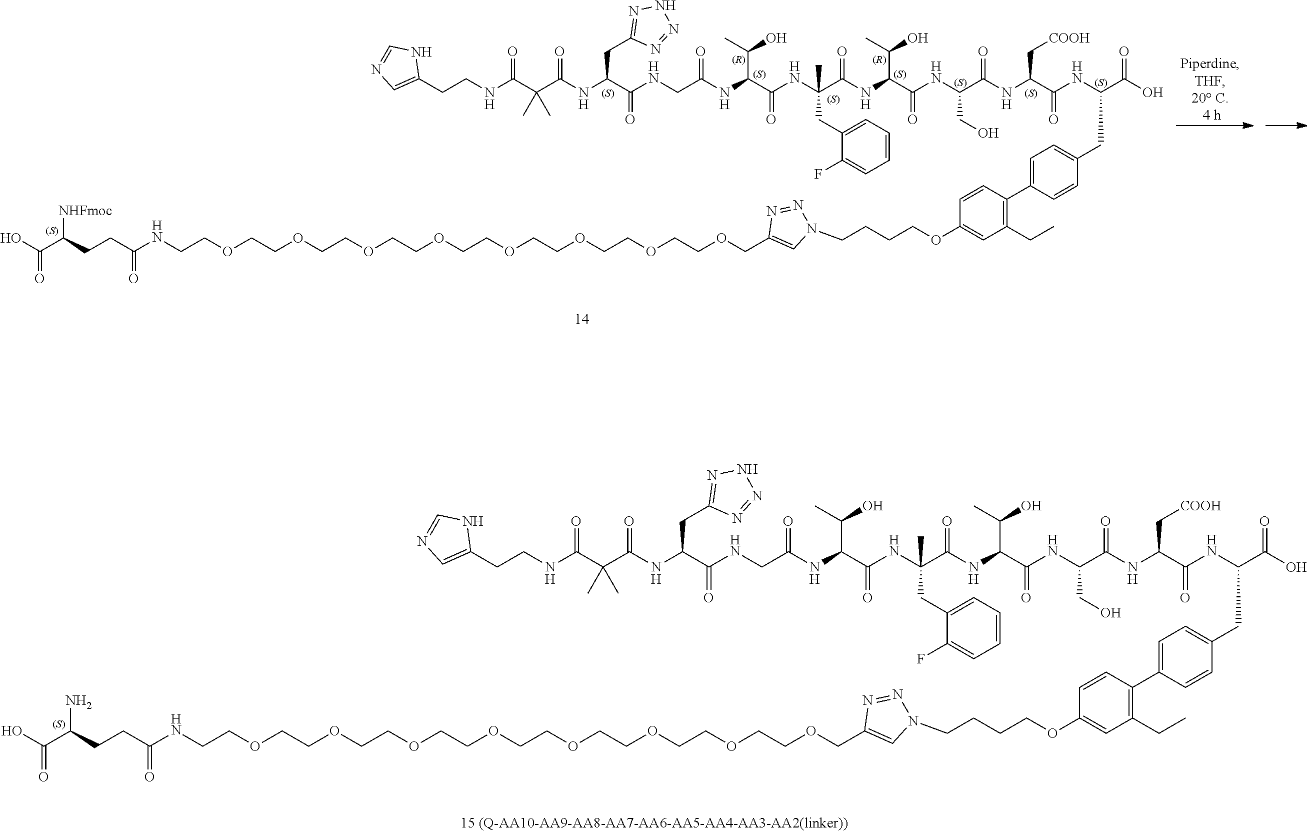

A solution of compound 14 (238 mg, 111.83 mol, 1 eq.) in DCM (5 mL) and piperidine (1 mL) was stirred at 25° C. for 12 hours. LCMS trace showed that the desired MS was detected. The solvent was removed under reduced pressure. The residue was purified by Prep-HPLC (column: X timate C18 150*40 mm*10 um; mobile phase: [water (NH₃H₂O+NH₄HCO₃)-ACN]; gradient: 7%-47% B over 9 min) and prep-HPLC (column: C18 150×40 mm; mobile phase: [water (NH₃H₂O+NH₄HCO₃)-ACN]; gradient: 0%-34% B over 9 min) again. Compound 15 (15 mg, 7.65 mol, 6.84% yield, 97.24% purity) was obtained as a white solid. 5.2 Purification and Identification of Compound 15

LCMS (ESI): RT=3.068 min, m/z calcd. for C₈₆H₁₂₇FN₂₀O₂₈, 953.45 [M+2H]2+; m/z found 954.00 [M+2H]²⁺. Mobile Phase: 1.5ML/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

HPLC AC RT=6.301 min, 97.24% purity. HPLC method A: Column: YMC-Pack ODS-A 150*4.6 mm, 5 μm; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 ml/min.

### Example 6. Synthesis of Compound 28 (AA10-AA9-AA8-AA7-AA6-AA5-AA4-AA3-(LLQ-L)AA2-OH) (SEQ ID NOS 262, 339-342, and 262 Disclosed Below, Respectively, in Order of Appearance)

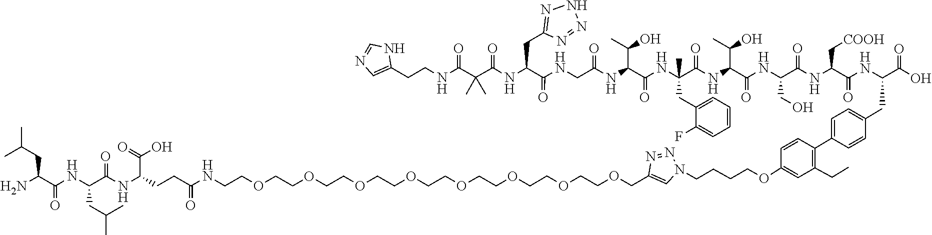

### 6.1 Synthesis, Cleavage, and Impurity Analysis of Compound 28

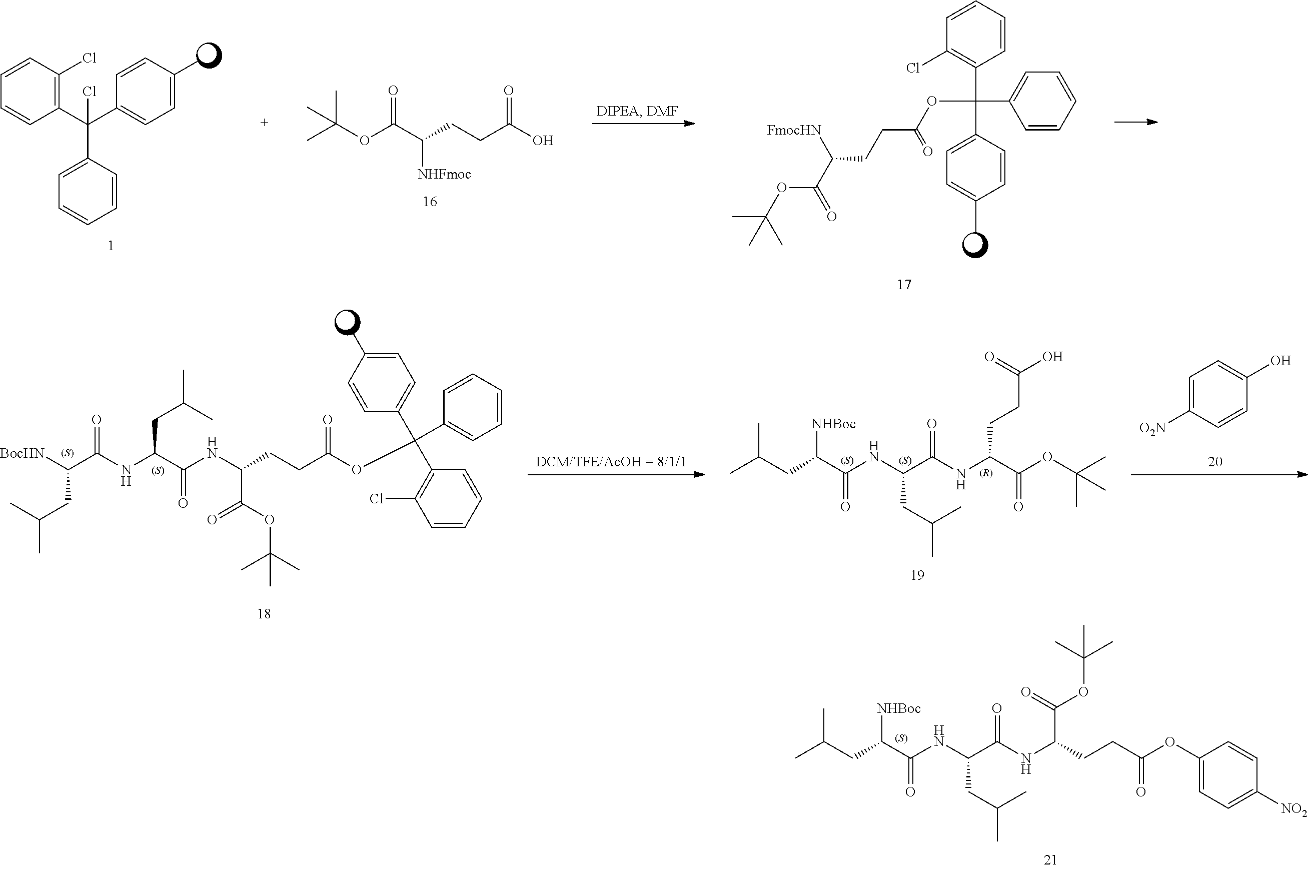

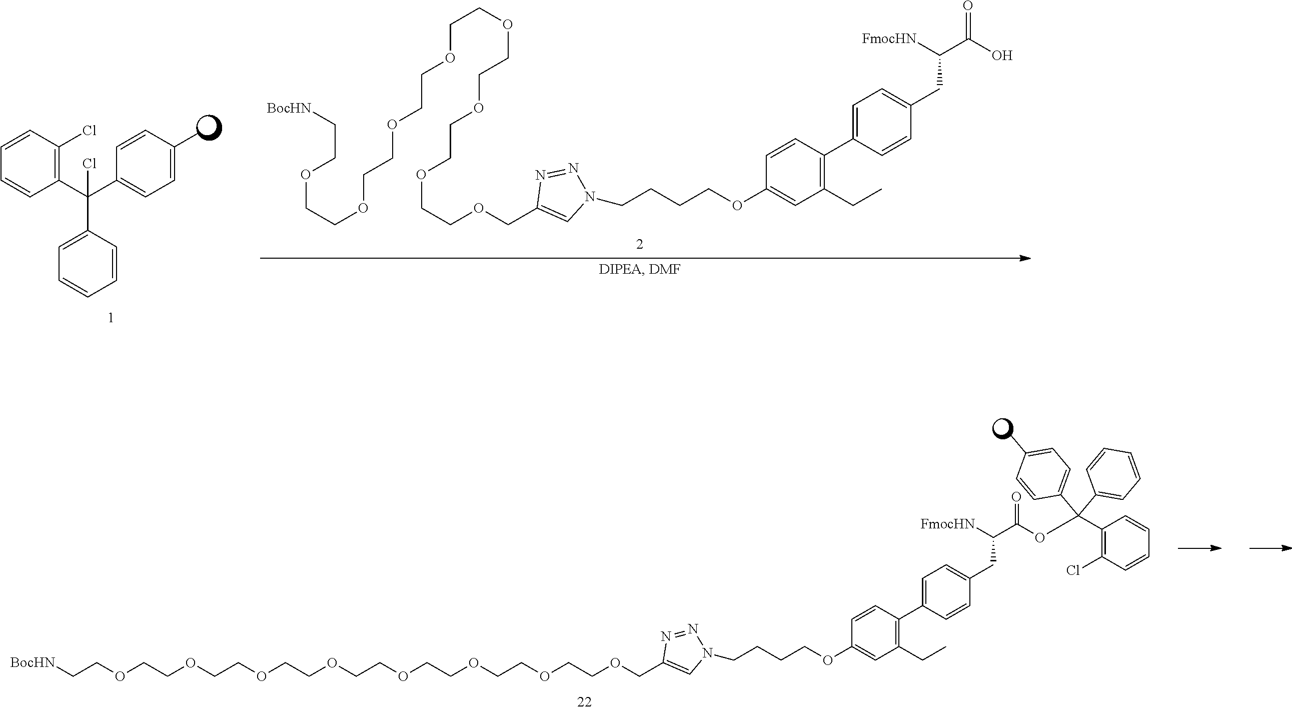

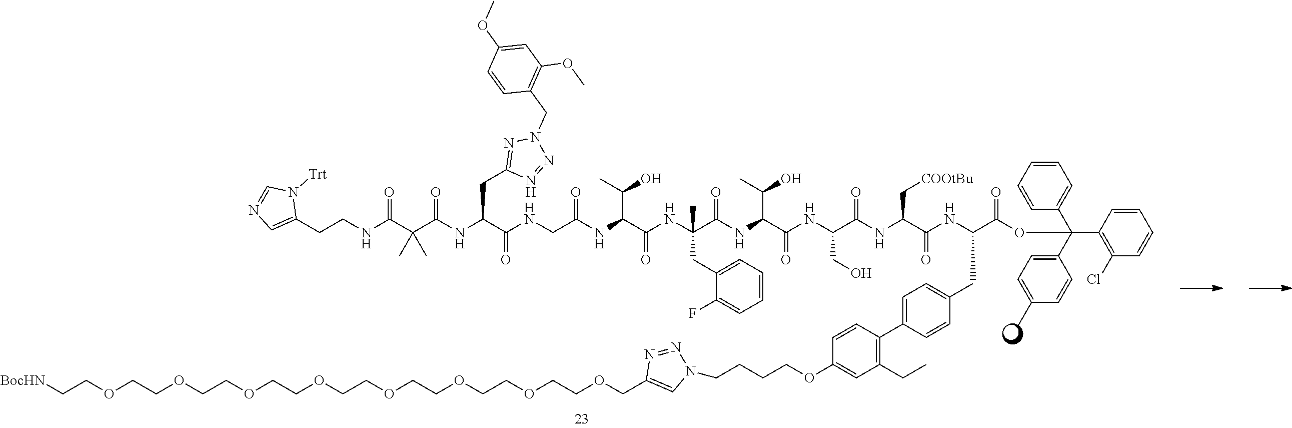

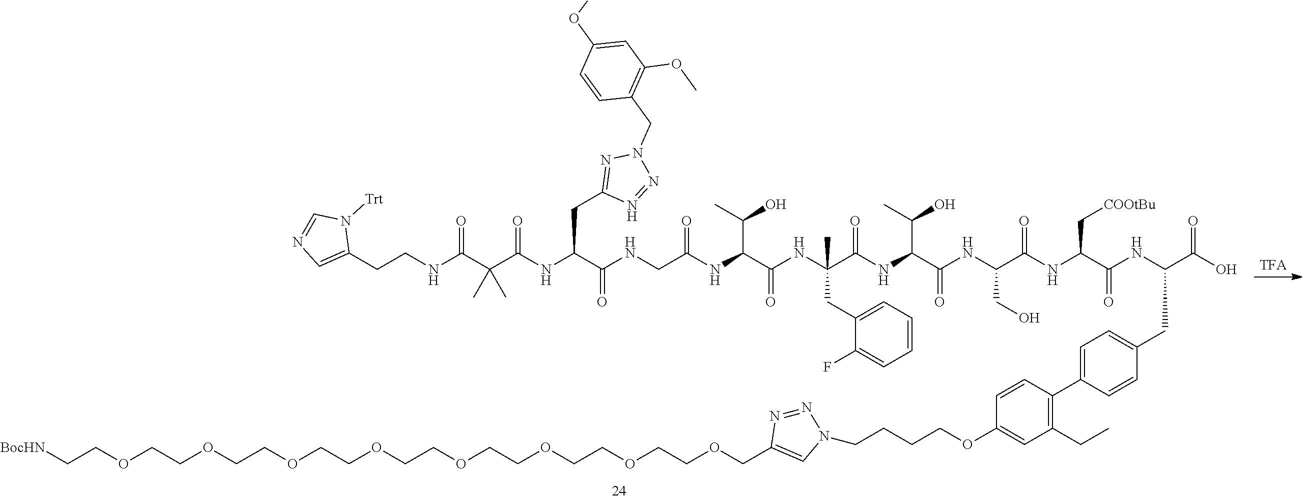

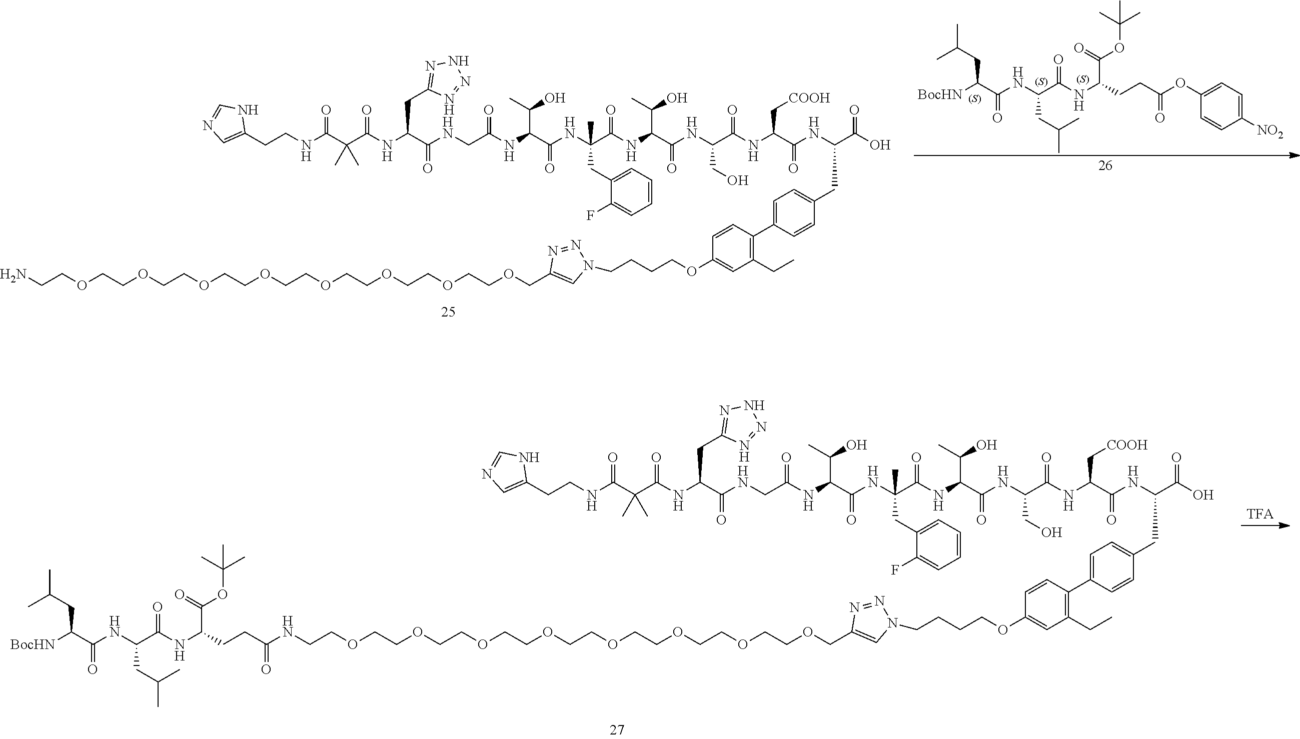

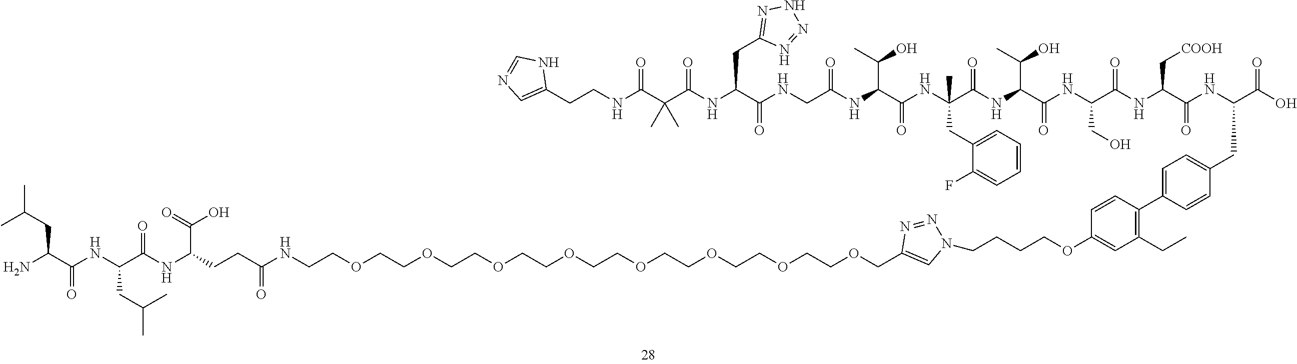

### Synthesis of Compound 21

### Step 1: Synthesis of O1-tert-Butyl 05-[(2-chlorophenyl)-diphenyl-methyl](2R)-2-(9H-fluoren-9-ylmethoxycarbonylamino)pentanedioate (17)

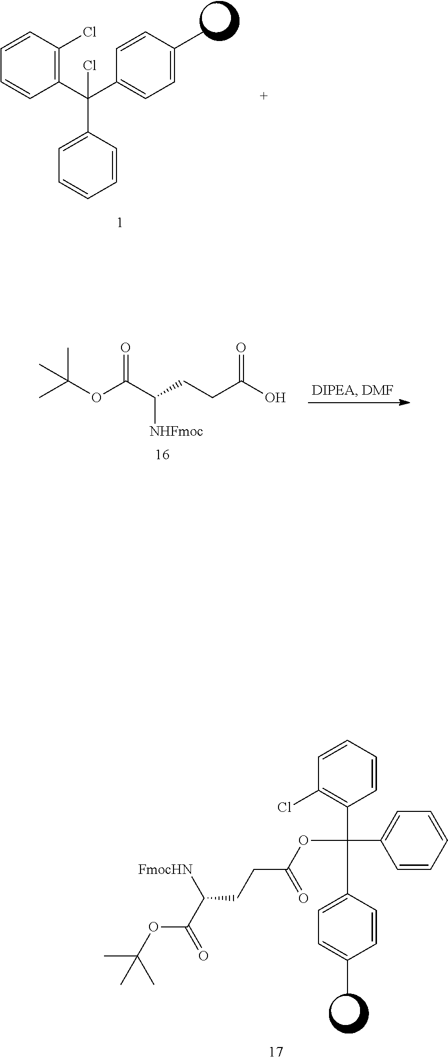

To a solution of resin (1) (1.25 g, 4 mmol, 1 eq.) in DMF (100 mL) were added compound 16 (8.51 g, 20.00 mmol, 5.0 eq.) and DIPEA (5.17 g, 40.00 mmol, 6.97 mL, 10.0 eq.). Then the mixture was shaken at 25° C. for 12 hours. MeOH (30 mL) was added and the mixture was shaken at 25° C. for another 2 hours. LCMS trace showed that the desired MS was detected. The mixture was filtered and the collected resin was washed with DMF (30 mL×3) and DCM (30 mL×3) to give the crude product on solid phase. Compound 17 (2.8 g, crude) was obtained as a yellow solid which was used directly in the next step.

### Step 2: Synthesis of O1-tert-Butyl O5-[(2-chlorophenyl)-diphenyl-methyl](2S)-2-[[(2S)-2-[[(2S)-2-(tert-butoxycarbonylamino)-4-methyl-pentanoyl]amino]-4-methyl-pentanoyl]amino]pentanedioate (18)

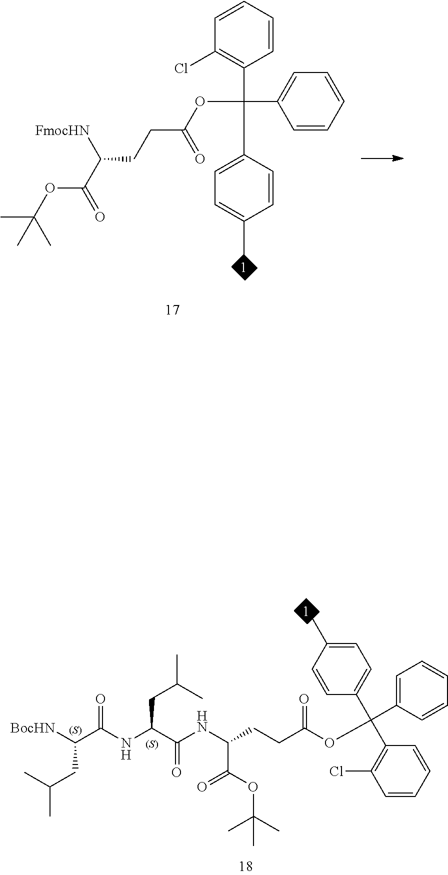

The peptide elongation was performed on a 0.25 mmol scale using liberty Blue Automated Microwave Peptide Synthesizer. Following the standard operation on a peptide synthesizer as follows. Step A: the 2-CTC resin compound 17 (2.80 g, 4 mmol, 1 eq.) was swollen with DMF (100 mL) for 300 seconds following the standard operation protocol of the peptide synthesizer. Step B (de-protection): a solution of 20% piperidine/DMF (100 mL) was added to the resin vessel and agitated with N₂ for 30 min at 30° C. The vessel was then drained and washed with DMF (3 mL×3) at 20° C. Step C (coupling) (each amino acid coupling reaction was repeated three times with 5.0 eq.): a solution of amino acid (20 mmol, 5 eq.) in DMF (5 mL), HATU (18 mmol, 4.5 eq.), and DIPEA (40 mmol, 10 eq.) were added to the vessel successively and agitated with N₂ for 60 min at 30° C. Steps B and C were repeated for all amino acids. The corresponding CTC resin compound 18 (3.22 g, crude) was prepared as described in the general procedure of SPPS. The crude product was obtained as a yellow solid which was used directly in the next step.

### Step 3: Synthesis of(4S)-5-tert-Butoxy-4-[[(2S)-2-[[(2S)-2-(tert-butoxycarbonylamino)-4-methyl-pentanoyl]amino]-4-methyl-pentanoyl]amino]-5-oxo-pentanoic Acid (19)

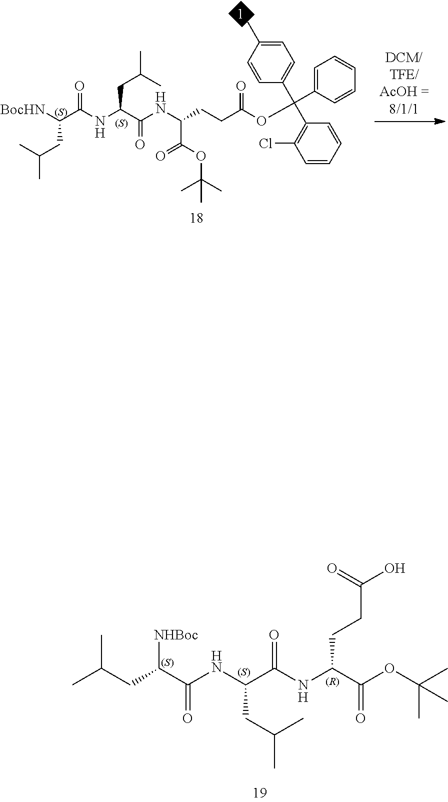

A solution of compound 18 (3.22 g, 4.0 mmol, 1 eq.) in DCM (80 mL), TFE (28.01 g, 279.97 mmol, 20.14 mL, 69.99 eq.), and AcOH (21.12 g, 351.73 mmol, 20.14 mL, 87.93 eq.) was stirred at 20° C. for 2 hours. LCMS showed the desired MS was detected. The solvent was removed under reduced pressure to give the residue. The residue was dissolved in H₂O (30 mL) and ACN (10 mL), then lyophilized to give the product. Compound 19 (1.1 g, 2.08 mmol, 51.92% yield) was obtained as a colorless oil.

LCMS (ESI): RT=4.309 min, m/z calcd. for C₂₆H₄₈N₃O₈, 530.24 [M+H]⁺, m/z found 530.30. Mobile Phase: 1.5 mL/4 L TFA in water (solvent A) and 0.75 mL/4 L TFA in acetonitrile (solvent B) using the elution gradient 10%-80% (solvent B) over 0.7 minutes and holding at 95% for 0.4 minutes at a flow rate of 1.5 mL/min. Column: Agilent Pursult 5 C18 20*2.0 mm. Wavelength: UV 220 nm and 254 nm. Column temperature: 50° C. MS ionization: ESI 50° C.

### Step 4: Synthesis of O1-tert-Butyl 05-(4-nitrophenyl) (2S)-2-[[(2S)-2-[[(2S)-2-(tert-butoxycarbonylamino)-4-methyl-pentanoyl]amino]-4-methyl-pentanoyl]amino]Pentanedioate (21)

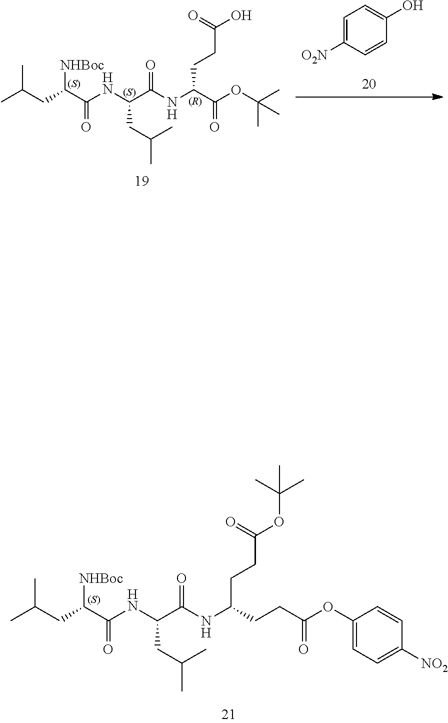

To a solution of compound 19 (1.1 g, 2.08 mmol, 1 eq.) in ACN (10 mL) were added 4-nitrophenol (20) (346.68 mg, 2.49 mmol, 1.2 eq.) and EDCI (477.75 mg, 2.49 mmol, 1.2 eq.). Then the mixture was stirred at 20° C. for 12 hours. LCMS trace showed that the desired MS was detected. The solvent was removed under reduced pressure. The residue was purified by prep-HPLC (column: Xtimate C18 150*40 mm*10 um; mobile phase: [water (FA)-ACN]; gradient: 46%-86% B over 9 min. Compound 21 (0.5 g, 768.33 mol, 37% yield, 100% purity) was obtained as a white solid.

LCMS (ESI): RT=5.751 min, m/z calcd. for C₃₂H₅₁N₄O₁₀, 651.35 [M+H]⁺, m/z found 651.50 [M+H]⁺, m/z found 1301.90 [2M+H]⁺. LC-MS method A: a MERCK, RP-18e 25-2 mm column, with a flow rate of 1.5 mL/min, eluting with a gradient of 10% to 80% acetonitrile containing 0.02% TFA (solvent B) and water containing 0.04% TFA (solvent A).

### Synthesis of Compound 28

### Step 1: Synthesis of [(2-Chlorophenyl)-diphenyl-methyl](2S)-3-[4-[4-[4-[4-[2-[2-[2-[2-[2-[2-[2-[2-(tertbutoxycarbonylamino)ethoxy]ethoxy]ethoxy]ethoxy]ethoxy]ethoxy]ethoxy]ethoxymethyl]triazol-1-yl]butoxy]-2-ethyl-phenyl]phenyl]-2-(9H-fluoren-9-ylmethoxycarbonylamino)propanoate (22)

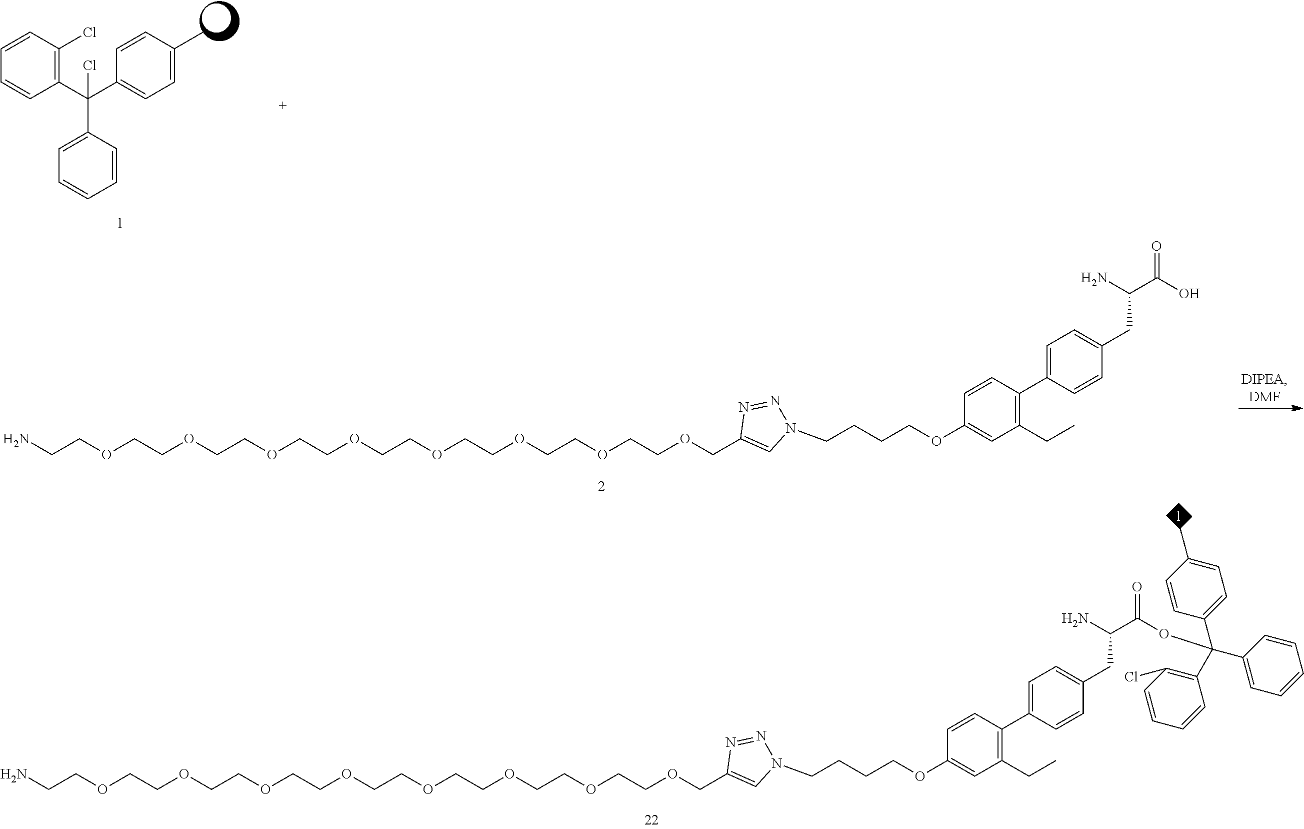 [SMILES: Cc1ccc(C(Cl)(c2ccccc2)c2ccccc2Cl)cc1 ; CCc1cc(OCCCCn2cc(COCCOCCOCCOCCOCCOCCOCCOCCN)nn2)ccc1-c1ccc(C[C@H](N)C(=O)O)cc1 ; CCc1cc(OCCCCn2cc(COCCOCCOCCOCCOCCOCCOCCOCCN)nn2)ccc1-c1ccc(C[C@H](N)C(=O)OC(c2ccccc2)(c2ccc(C)cc2)c2ccccc2Cl)cc1]

To a solution of resin (1) (312.21 mg, 1 mmol, 1 eq.) in DMF (100 mL) were added compound 2 (3.34 g, 3.00 mmol, 1.5 eq.) and DIPEA (1.29 g, 10.00 mmol, 1.74 mL, 10.0 eq.). Then the mixture was shaken at 25° C. for 12 hours. MeOH (30 mL) was added and the mixture was shaken at 25° C. for another 2 hours. LCMS trace showed that the desired MS was detected. The mixture was filtered, and the collected resin was washed with DMF (30 mL×3) and DCM (30 mL×3) to give the crude product on solid phase. Compound 22 (2.78 g, 2.00 mmol, 100.00% yield) was obtained as a yellow solid which was used directly in the next step.

### Step 2: Synthesis of tert-Butyl (3S)-4-[[(]S)-1-[[4-[4-[4-[4-[2-[2-[2-[2-[2-[2-[2-[2-(tert- butoxycarbonylamino)ethoxy]ethoxy]ethoxy]ethoxy]ethoxy]ethoxy]ethoxy]ethoxymethyl]triazol-1-yl]butoxy]-2-ethyl-phenyl]phenyl]methyl]-2-[(2-chlorophenyl)-diphenyl-methoxy]-2-oxo-ethyl]amino]-3-[[(2S)-2-[[(2S,3R)-2-[[2-[[(2S,3R)-2-[[2-[[(2S)-3-[ ]-[(2,4-dimethoxyphenyl) methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoate (23) (SEQ ID NO: 339) (SEQ ID NO 339 Disclosed Below)

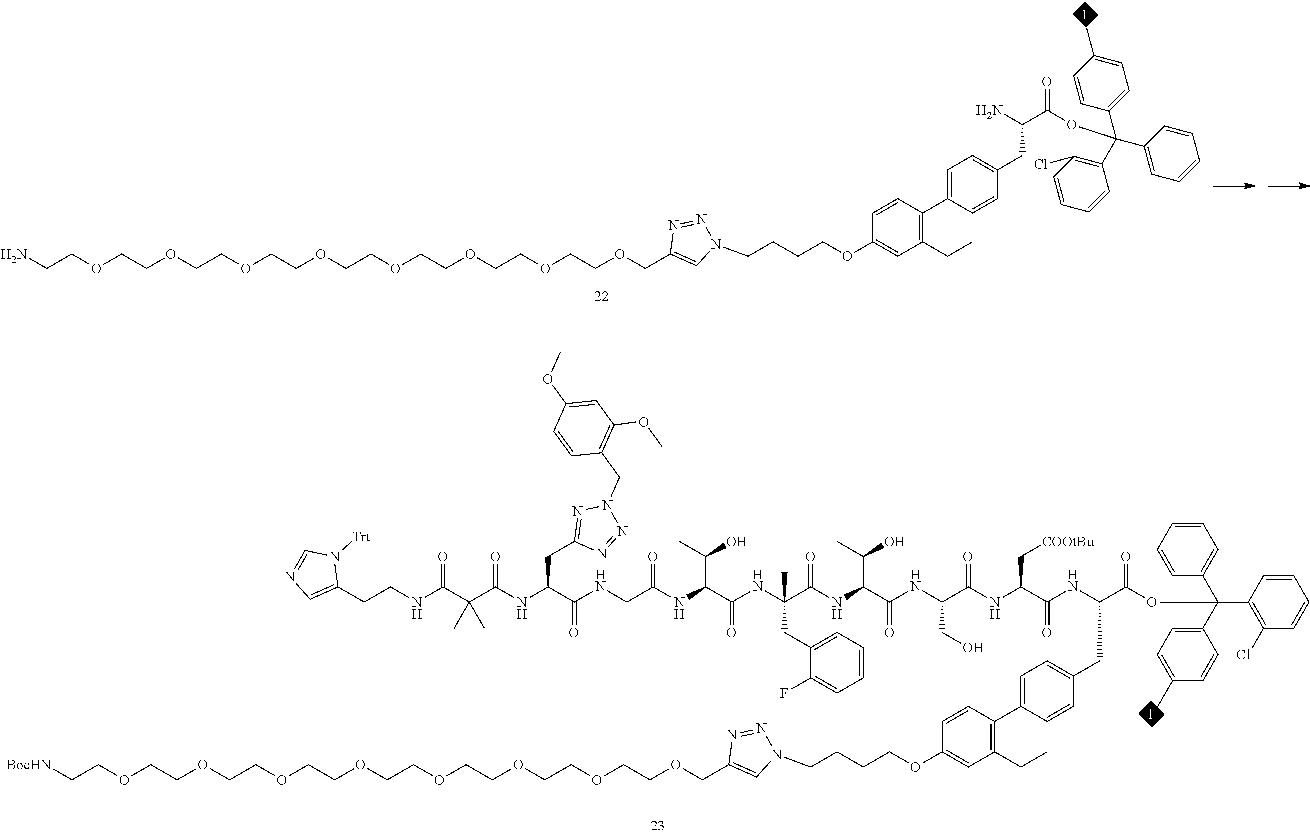

The peptide elongation was performed on a 0.25 mmol scale using liberty Blue Automated Microwave Peptide Synthesizer. Following the standard operation on a peptide synthesizer as follows. Step A: the 2-CTC resin compound 22 (0.25 mmol, 1 eq.) was swollen with DMF (10 mL) for 300 seconds following the standard operation protocol of the peptide synthesizer. Step B (de-protection): a solution of 20% piperidine/DMF (5 mL) was added to the resin vessel and the mixture was agitated with N₂ for 2 min at 90° C. The vessel was drained and washed with DMF (3 mL×3) at 20° C. Step C (coupling) (each amino acid coupling reaction was repeated three times with 5.0 eq.): a solution of amino acid (2.5 mmol, 5 eq.) in DMF (5 mL), DIC (2 mL), and Oxyma (1 mL) were added to the vessel successively and agitated with N₂ for 10 min at 90° C. Steps B and C were repeated for all amino acids. Compound 23 (2.84 g, 998.70 mol, 100.00% yield) was prepared as described in the general procedure of SPPS. The crude product was obtained as a yellow solid which was used directly in the next step.

### Step 3: Synthesis of (2S)-3-[4-[4-[4-[4-[2-[2-[2-[2-[2-[2-[2-(2-Aminoethoxy)ethoxy]ethoxy]ethoxy]ethoxy]ethoxy]ethoxy]ethoxymethyl]triazol-1-yl]butoxy]-2-ethyl-phenyl]phenyl]-2-[[(2S)-4-tert-butoxy-2-[[(2S)-2-[[(2S,3R)-2-[[2-[[(2S,3R)-2-[[2-[[(2S)-3-[1-[(2,4-dimethoxyphenyl) methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoyl]amino]propanoic Acid (24) (SEQ ID NO: 340) (SEQ ID NOS 339-340 Disclosed Below, Respectively, in Order of Appearance)

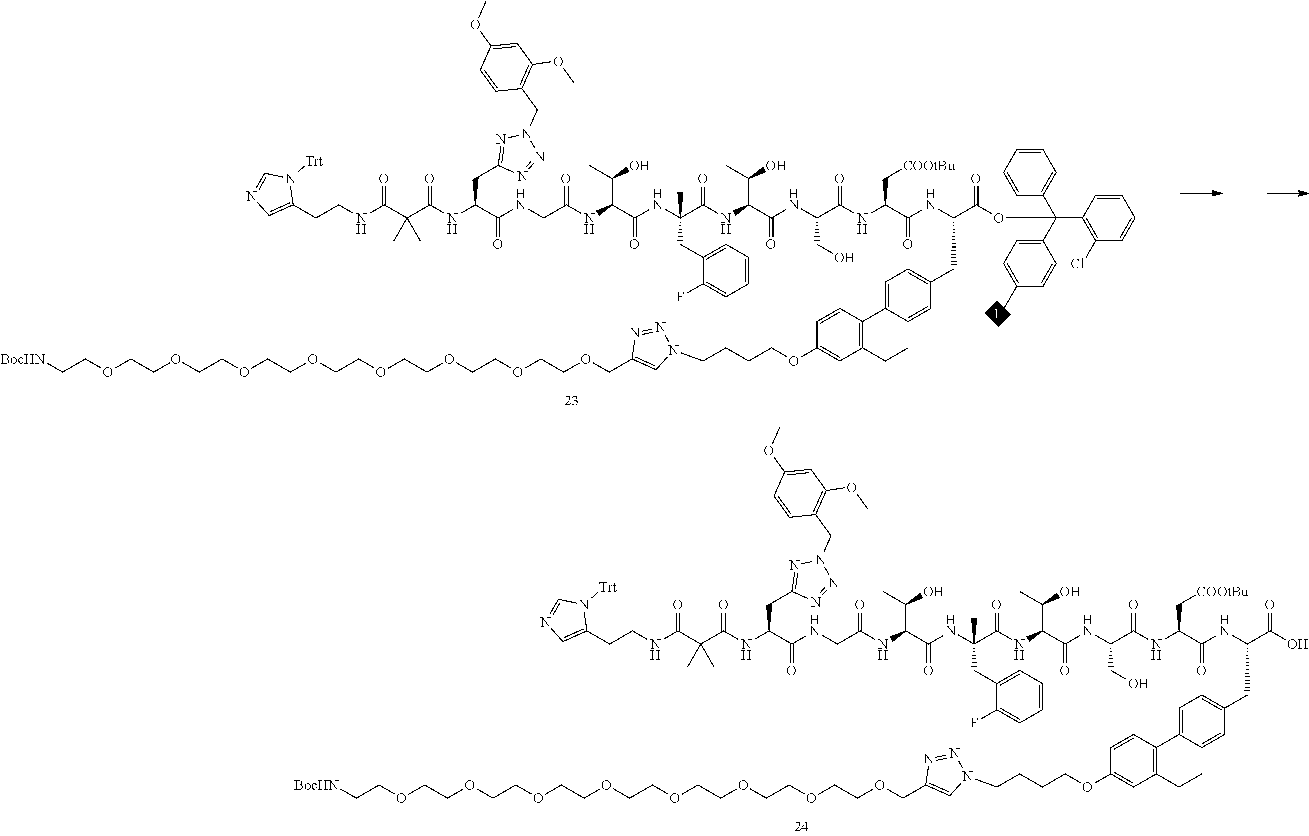

A solution of compound 23 (2.84 g, 998.70 mol, 1 eq.) in DCM (200 mL), TFE (25 mL), and AcOH (25 mL) was stirred at 20° C. for 2 hours. LCMS trace showed that the reaction was driven to completion and the desired MS was detected. The mixture was filtered and the filtrate was concentrated under low pressure. Compound 24 (2.46 g, 996.83 mol, 100.00% yield) was obtained as a white solid which was used directly in the next step.

LCMS RT=5.756 min, m/z calcd. for C₁₃₇H₁₇₄FN₁₉O₂₉, 1284.13 [M+2H]²⁺, m/z found 1285.00. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Step 4: Synthesis of (3S)-4-[[(S)-2-[4-[4-[4-[4-[2-[2-[2-[2-[2-[2-[2-(2-Aminoethoxy)ethoxy]ethoxy]ethoxy]ethoxy]ethoxy]ethoxy]ethoxymethyl]triazol-1-yl]butoxy]-2-ethyl-phenyl]phenyl]-1-carboxy-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(]H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic Acid (25) (SEQ ID NO: 341) (SEQ ID NOS 340-341 Disclosed Below, Respectively, in Order of Appearance)

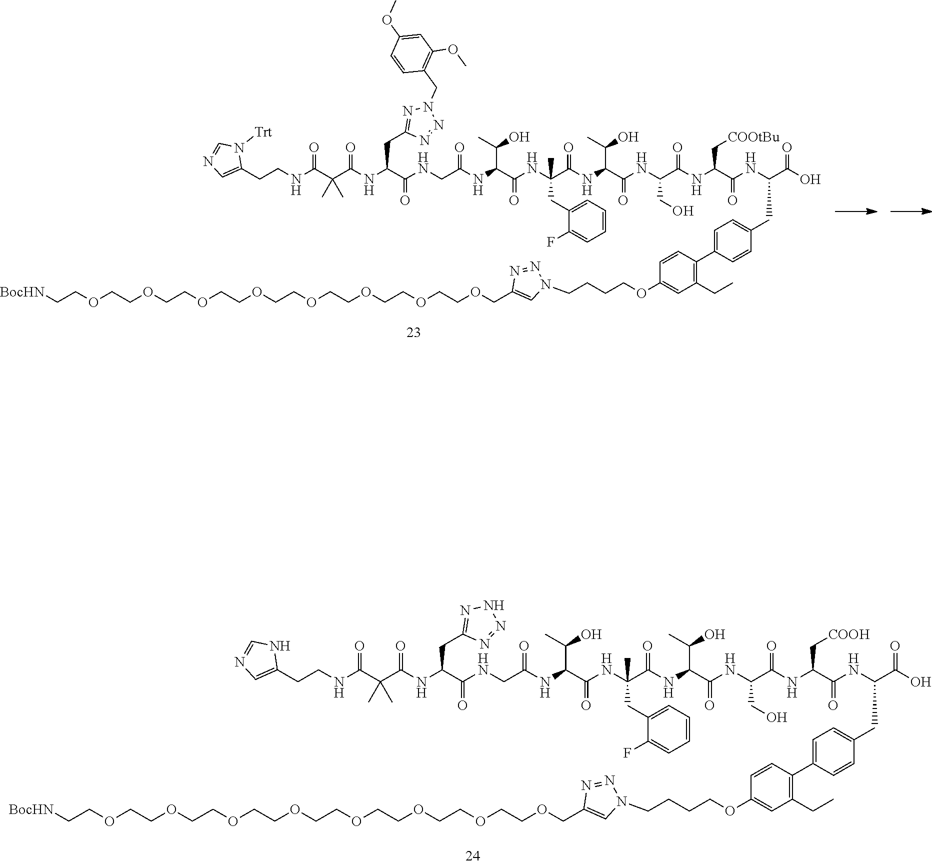

A solution of compound 24 (2.47 g, 1 mmol, 1 eq) in TFA (38 ml), triisopropylsilane (1 mL) and H₂O (1 mL) was stirred at 20° C. for 2 hours. LCMS trace showed that the desired MS was detected. The reaction mixture was triturated with MTBE (200 mL) and the solid was precipitated to give the crude product (1.8 g). The residue was purified by prep-HPLC (column: C18 150×30 mm; mobile phase: [water (TFA)-ACN]; gradient: 10%-50% B over 9 min) and prep-HPLC (column: C18 150×40 mm; mobile phase: [water (NH₃H₂O+NH₄HCO₃)-ACN]; gradient: 0%-37% B over 9 min. Compound 25 (0.5 g, 248.38 mol, 12.42% yield, 88.27% purity) was obtained as a white solid.

LCMS RT=2.944 min, m/z calcd. for C₈₁H₁₂₀FN₁₉O₂₅, 888.93 [M+2H]²⁺, m/z found 889.50. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

HPLC AC RT=6.346 min, 88.27% purity. HPLC method A: Column: YMC-Pack ODS-A 150*4.6 mm, Sum; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 ml/min.

### Step 5: Synthesis of (3S)-4-[[(]S)-2-[4-[4-[4-[4-[2-[2-[2-[2-[2-[2-[2-[2-[[(4S)-5- tert-Butoxy-4-[[(2S)-2-[[(2S)-2-(tert-butoxycarbonylamino)-4-methyl-pentanoyl]amino]-4-methyl-pentanoyl]amino]-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]-1-carboxy-ethyl]amino]-3-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]-4-oxo-butanoic Acid (27) (SEQ ID NO: 342) (SEQ ID NOS 341-342 Disclosed Below, Respectively, in Order of Appearance)

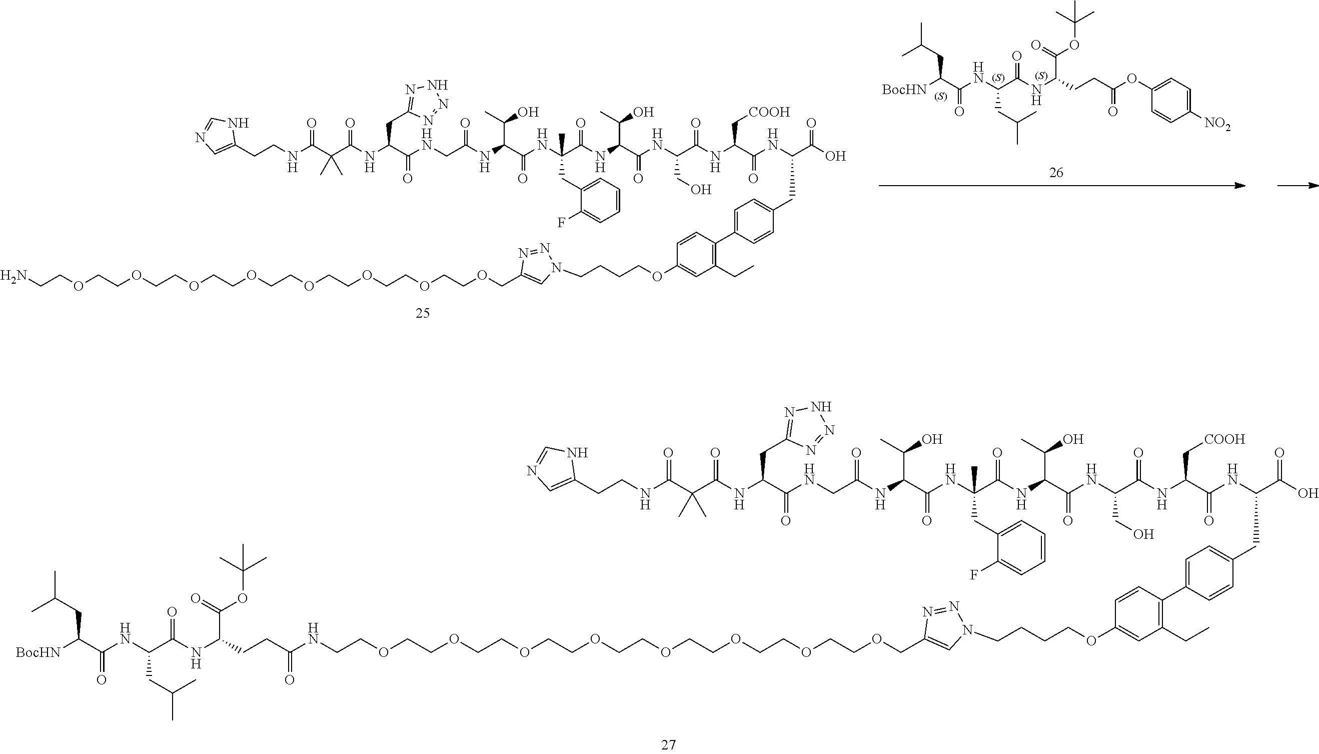

To a solution of compound 25 (200 mg, 112.55 mol, 1 eq.) in DMF (3 mL) were added compound 26 (80.57 mg, 123.81 mol, 1.1 eq.) and DIPEA (29.09 mg, 225.11 mol, 39.21 μL, 2.0 eq.). Then the solution was stirred at 20° C. for 12 hours. LCMS trace showed that the desired MS was detected. The solvent was removed under reduced pressure. Compound 27 (257 mg, crude) was obtained as a yellow oil which was used directly in the next step.

LCMS (ESI): RT=4.459 min, m/z calcd. for: C₁₀₇H₁₆₅FN₂₂O₃₂, 1145.59 [M+2H]2+; m/z found 1145.30 [M+2H]²⁺, 763.90[M+3H]³⁺, Mobile Phase: 1.5 mL/4 L TFA in water (solvent A) and 0.75 mL/4 L TFA in acetonitrile (solvent B) using the elution gradient 5%-95% (solvent B) over 0.7 minutes and holding at 95% for 0.4 minutes at a flow rate of 1.5 mL/min. Column: Agilent Pursult 5 C18 20*2.0 mm. Wavelength: UV 220 nm and 254 nm. Column temperature: 50° C. MS ionization: ESI 50° C.

### Step 6: Synthesis of (2S)-2-[[(2S)-2-[[(2S)-2-Amino-4-methyl-pentanoyl]amino]-4-methyl-pentanoyl]amino]-5-[2-[2-[2-[2-[2-[2-[2-[2-[[ ]-[4-[4-[4-[(2S)-2-carboxy-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H- imidazol-5-yl)ethylamino]-2,2-dimethyl-3-oxo-propanoyl]amino]-3-(2H-tetrazol-5-yl) propanoyl]amino]acetyl]amino]butanoyl]amino]-2-methyl-propanoyl]amino]-3-hydroxy-butanoyl]amino]-3-hydroxy-propanoyl]amino]propanoyl]amino]ethyl]phenyl]-3-ethyl-phenoxy]butyl]triazol-4-yl]methoxy]ethoxy]ethoxy]ethoxy]ethoxy]ethoxy]ethoxy]ethoxy]ethylamino]-5-oxo-pentanoic Acid (28) (SEQ ID NO: 262) (SEQ ID NOS 342 and 262 Disclosed Below, Respectively, in Order of Appearance)

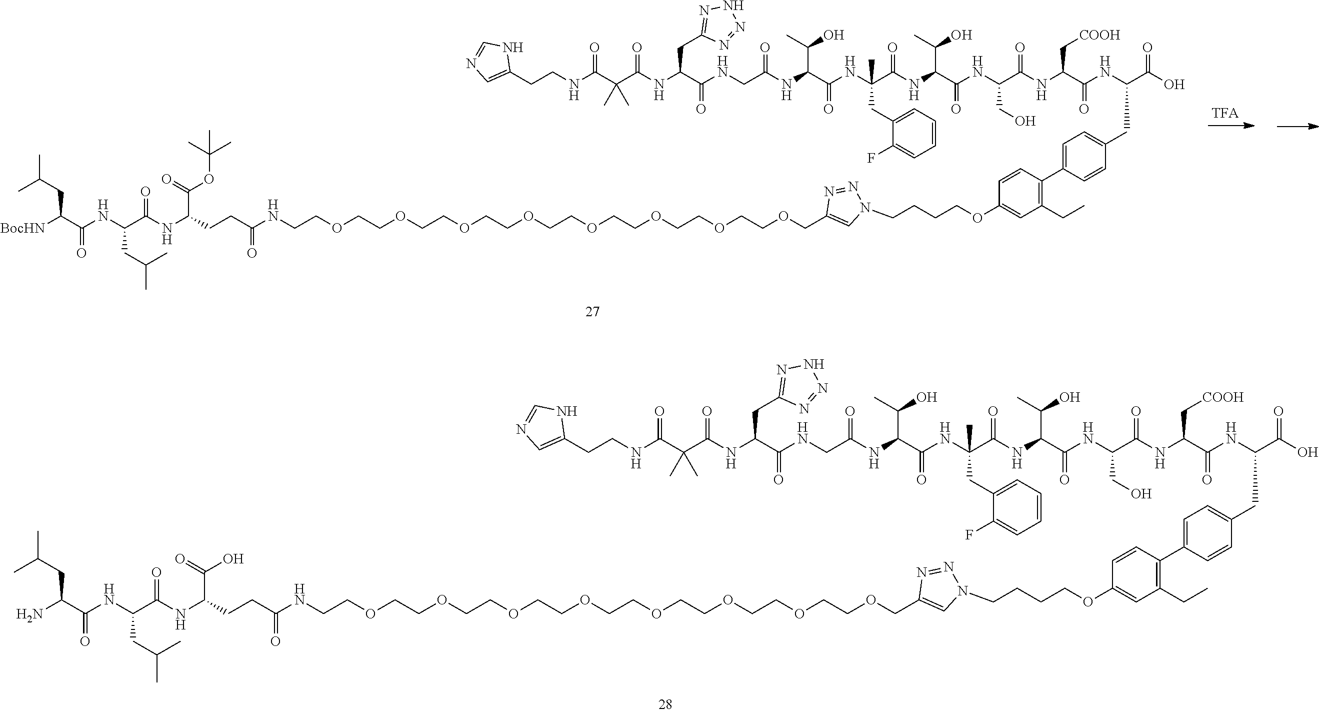

A solution of compound 27 (257 mg, 112.30 mol, 1 eq.) in DCM (5 mL) and TFA (5 mL) was stirred at 25° C. for 12 hours. LCMS trace showed that the desired MS was detected. The solvent was removed under reduced pressure. The residue was purified by prep-HPLC (column: C18 150×30 mm; mobile phase: [water (TFA)-ACN]; gradient: 10%-50% B over 9 min) and prep-HPLC (column: C18 150×40 mm; mobile phase: [water (NH₃H₂O+NH₄HCO₃)-ACN]; gradient: 0%-36% B over 9 min) again. Compound 28 (13 mg, 5.80 mol, 5.17% yield, 95.15% purity) was obtained as a white solid. 6.2 Purification and Identification of Compound 28

LCMS (ESI): RT=3.242 min, m/z calcd. for C₉₈H₁₄₉FN₂₂O₃₀, 1066.53 [M+2H]²⁺; m/z found 1067.20 [M+2H]²⁺. Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

HPLC RT=6.641 min, 95.15% purity. HPLC method A: Column: YMC-Pack ODS-A 150*4.6 mm, Sum; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 ml/min.

### Example 7. Synthesis of Compound 33 (AA10-AA9-AA8-AA7-AA6-AA5-AA4-AA3-AA2-AA1-OH) (SEQ ID NO: 343 Disclosed Below)

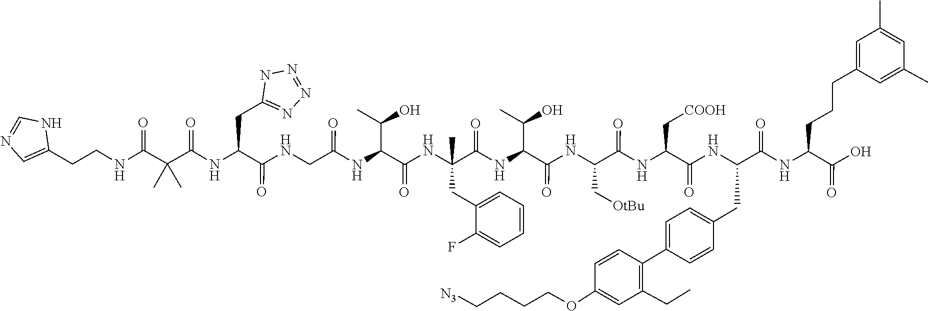

### 7.1 Synthesis, Cleavage, and Impurity Analysis of Compound 33 (SEQ ID NO: 343 Disclosed Below)

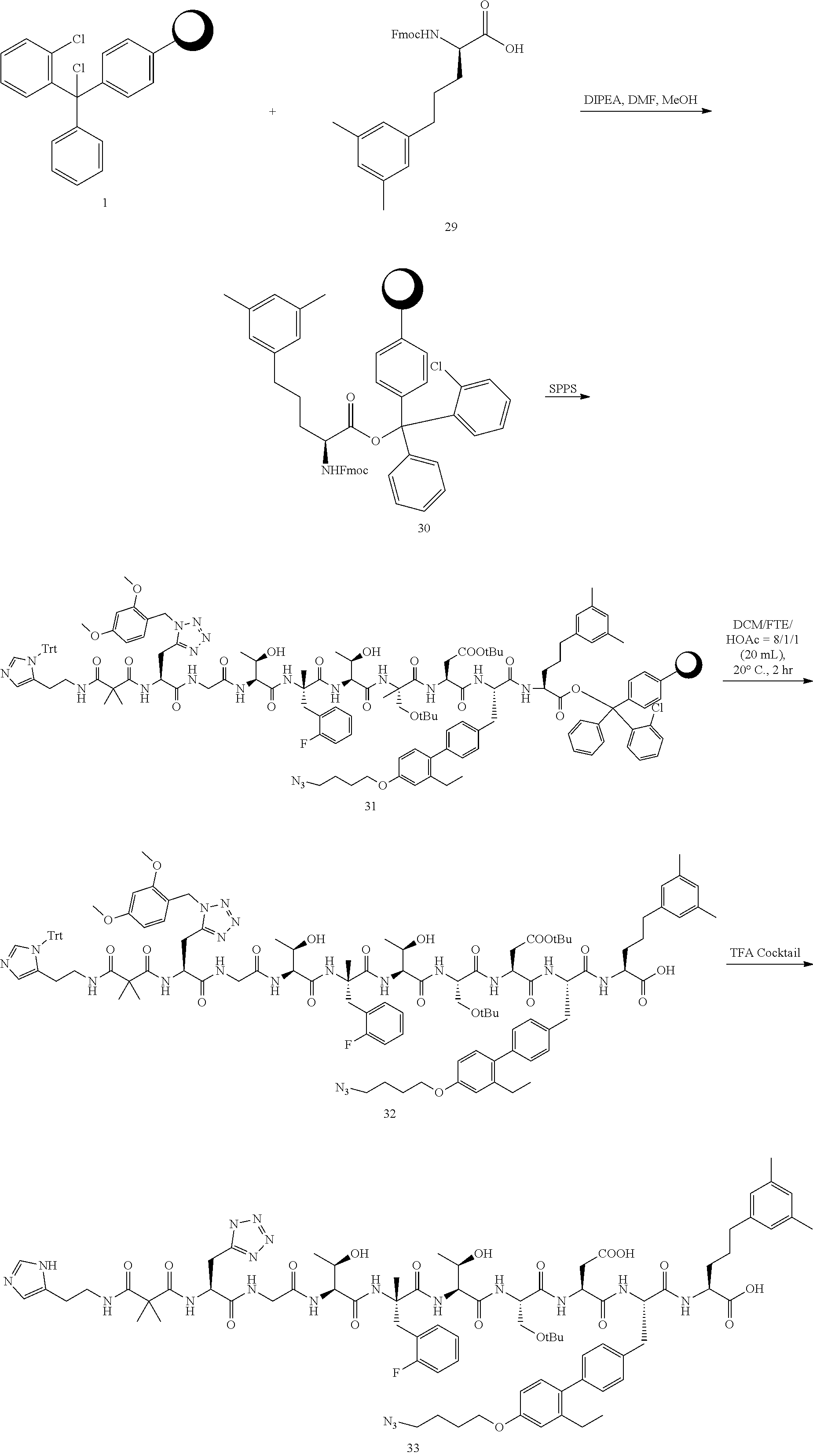

### Step 1: Synthesis of [(2-Chlorophenyl)-diphenyl-methyl](2S)-5-(3,5-dimethylphenyl)-2-(9H-fluoren-9-ylmethoxycarbonylamino)pentanoate (30)

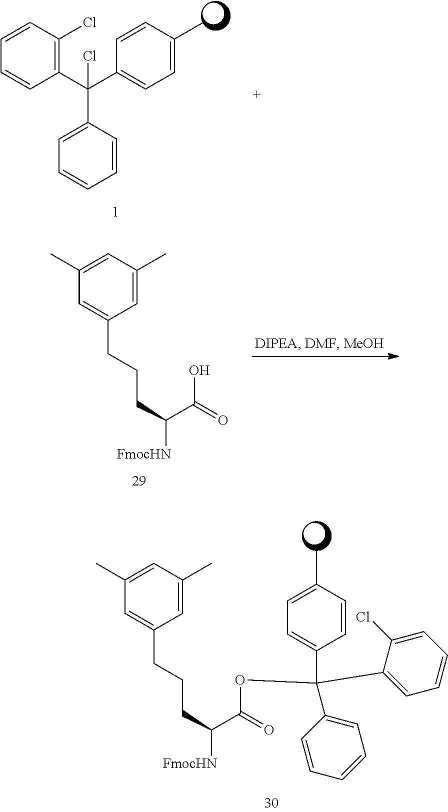

To a solution of resin (1) (312.21 mg, 0.47 mmol, 1 eq.) in DMF (30 mL) compound 29 (698.67 mg, 2.35 mmol, 5.0 eq.) and DIPEA (607.44 mg, 4.70 mmol, 818.66 L, 10.0 eq.) were added. Then the mixture was shaken at 25° C. for 12 hours. MeOH (10 mL) was added and the mixture was shaken at 25° C. for another 2 hours. The mixture was filtered and the collected resin was washed with DMF (30 mL×3) and DCM (30 mL×3) to give the crude product on solid phase. Compound 30 (0.18 g, crude) was obtained as a yellow solid which was used directly in the next step.

### Step 2: Synthesis of [(2-Chlorophenyl)-diphenyl-methyl](2S)-2-[[(2R)-3-[4-[4-(4-azidobutoxy)-2-ethyl-phenyl]phenyl]-2-[[(2S)-4-tert-butoxy-2-[[(2S)-2-[[(2S,3R)-2-[[(2R)-2-[[(2S,3R)-2-[[2-[[(2R)-3-[2-[(2,4-dimethoxyphenyl)methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoyl]amino]propanoyl]amino]-5-(3,5-dimethylphenyl)pentanoate (31)

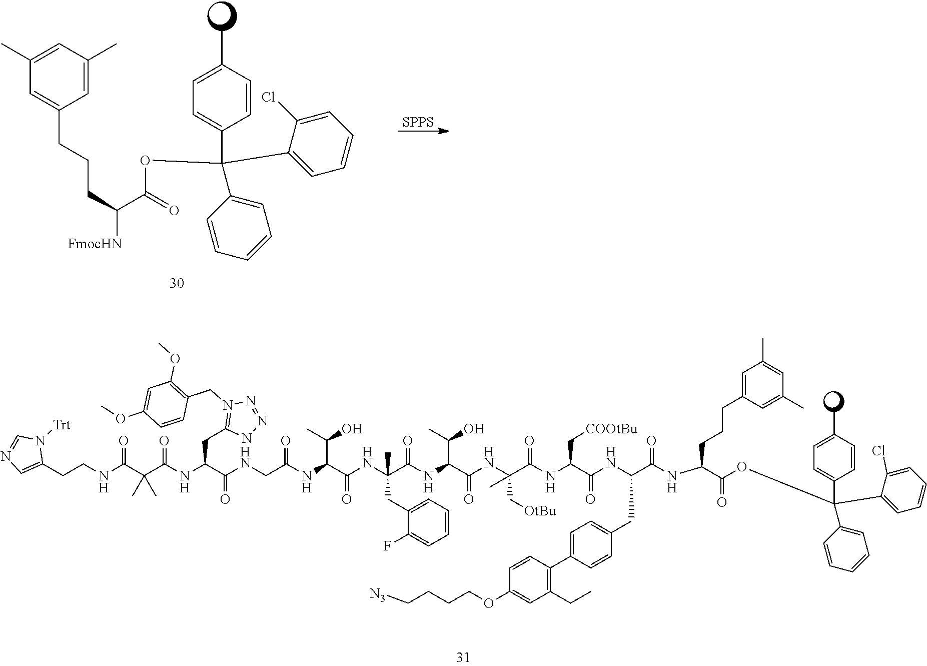

Compound 30 (179.82 mg, 0.25 mmol, 1 eq.) in 20% piperidine in DMF (4 mL) was bubbled with N₂ at 20° C. for 0.5 hours. The mixture was filtered and the collected resin was washed with DMF (100 mL×3) and DCM (100 mL×3) to give the crude product on solid phase. Then a solution of (2S)-3-[4-[4-(4-azidobutoxy)-2-ethyl-phenyl]phenyl]-2-(9H-fluoren-9-ylmethoxy carbonylamino) propanoic acid (453.50 mg, 0.75 mmol, 3.0 eq), HATU (532.32 mg, 1.40 mmol, 653.15 μL, 2.8 eq.), and DIPEA (387.73 mg, 3.00 mmol, 522.54 μL, 6.0 eq.) in 20% piperidine in DMF (4 mL) was added and the mixture was bubbled with N₂ at 20° C. for 1 hour. Other amino acids were repeated according the above procedure. Then the mixture was filtered and the collected resin was washed with DMF (50 mL×3) and DCM (50 mL×3) to give compound 31 (0.6 g, crude).

### Step 3: Synthesis of (2S)-2-[[(2R)-3-[4-[4-(4-Azidobutoxy)-2-ethyl-phenyl]phenyl]-2-[[(2S)-4-tert-butoxy-2-[[(2S)-2-[[(2S,3R)-2-[[(2R)-2-[[(2S,3R)-2-[[2-[[(2R)-3-[2-[(2,4-dimethoxyphenyl) methyl]tetrazol-5-yl]-2-[[2,2-dimethyl-3-oxo-3-[2-(3-tritylimidazol-4-yl)ethylamino]propanoyl]amino]propanoyl]amino]acetyl]amino]-3-hydroxy-butanoyl]amino]-3-(2-fluorophenyl)-2-methyl-propanoyl]amino]-3-hydroxy-butanoyl]amino]-3-trityloxy-propanoyl]amino]-4-oxo-butanoyl]amino]propanoyl]amino]-5-(3,5-dimethylphenyl)pentanoic Acid (32)

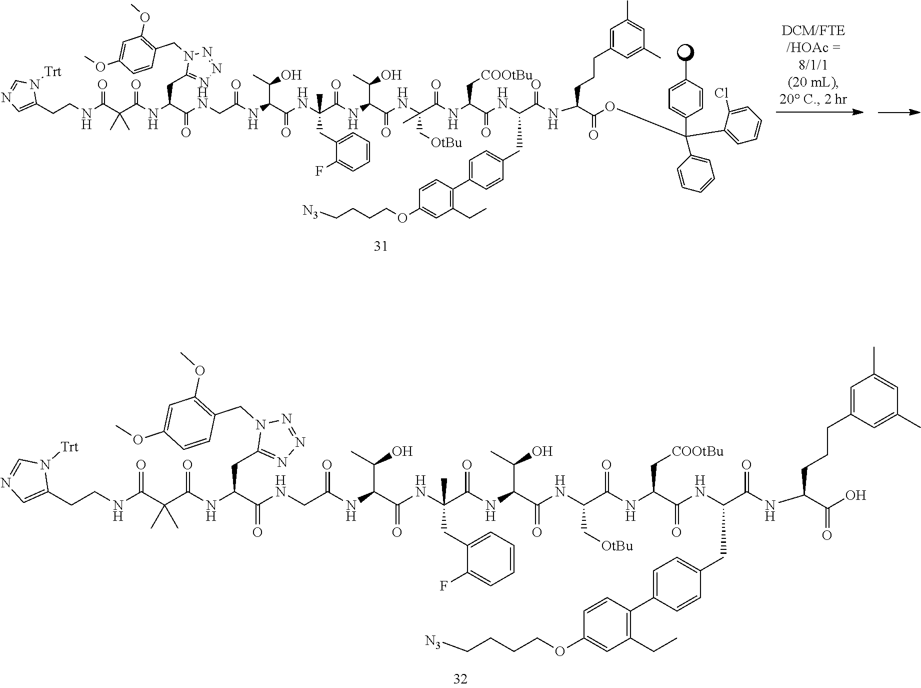

A solution of compound 31 (634.84 mg, 0.25 mmol, 1 eq.) in DCM (16 mL), TFE (2 mL), and HOAc (2 mL) was stirred at 20° C. for 2 hours. LCMS showed that the desired MS was detected. The mixture reaction was filtered and the filtrate was concentrated under reduced pressure. Compound 32 (565 mg, crude) was obtained as a yellow oil which was used directly in the next step.

LCMS (ESI): RT=5.728 min, m/z calcd. for C₁₂₆H₁₄₅FN₁₉O₂₀Na 1143.03 [M+Na]⁺, m/z found 1143.70. Mobile Phase: 1.5 ML/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Step 4: Synthesis of (2S)-2-[[(2S)-3-[4-[4-(4-Azidobutoxy)-2-ethyl-phenyl]phenyl]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]propanoyl]amino]-5-(3,5-dimethylphenyl)pentanoic Acid (33) (SEQ ID NO: 343) (SEQ ID NO: 343 Disclosed Below)

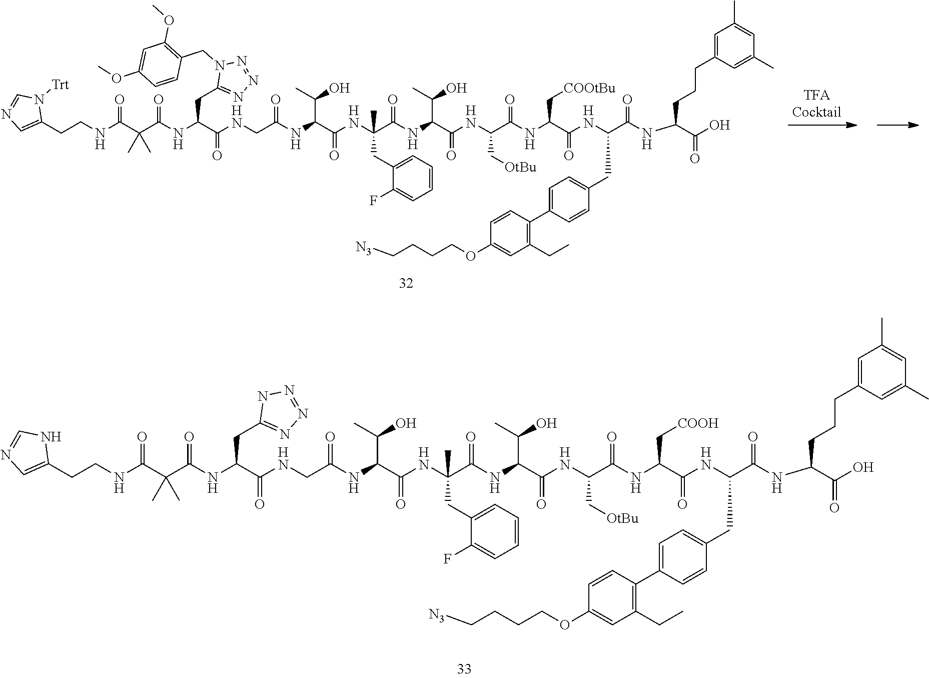

A solution of compound 32 (565.90 mg, 0.25 mmol, 1 eq.) in TFA cocktail reagent (15 mL, 2.5% H₂O, 7.5% 3-MPA, 2.5% TIS, and 87.5% TFA) was stirred at 20° C. for 2 hours. LCMS trace showed that the desired MS was detected. The solvent was removed under reduced pressure. The residue was purified by prep-HPLC (column: C18 150×30 mm; mobile phase: [water (TFA)-ACN]; gradient: 16%-56% B over 9 min) and prep-HPLC (column: C18 150×40 mm; mobile phase: [water (NH₃H₂O+NH₄HCO₃)-ACN]; gradient: 7%-47% B over 9 min) again. Compound 33 (20 mg, 12.55 mol, 5.02% yield, 98.70% purity) was obtained as a white solid. 7.2 Purification and Identification of Compound 33

LCMS (ESI): RT=4.867 min, m/z calcd. for C₇₅H₁₀₀FN₁₉O₁₈, 786.87 [M+2H]²⁺; m/z found 787.40 [M+2H]²⁺. Mobile Phase: 1.5ML/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

HPLC RT=9.514 min, 98.70% purity. HPLC method A: Column: YMC-Pack ODS-A 150*4.6 mm, 5 μm; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 ml/min.

### Example 8. Synthesis of LP 69 (AA10-AA9-AA8-AA7-AA6-AA5-AA4-AA3-(Q-L)AA2-AA1-OH) (SEQ ID NO: 120 Disclosed Below)

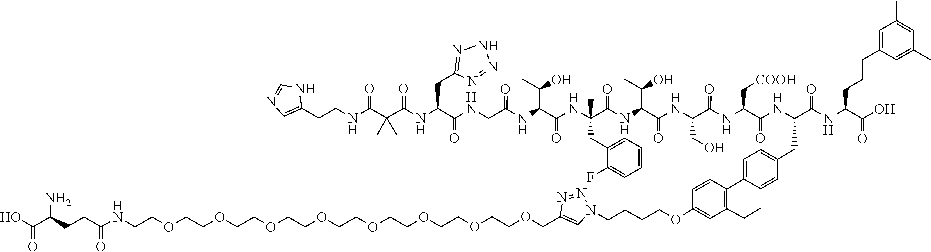

### 8.1 Synthesis, Cleavage, and Impurity Analysis of LP 69 (SEQ ID NOS 119, 344-345, and 120 Disclosed Below, Respectively, in Order of Appearance)

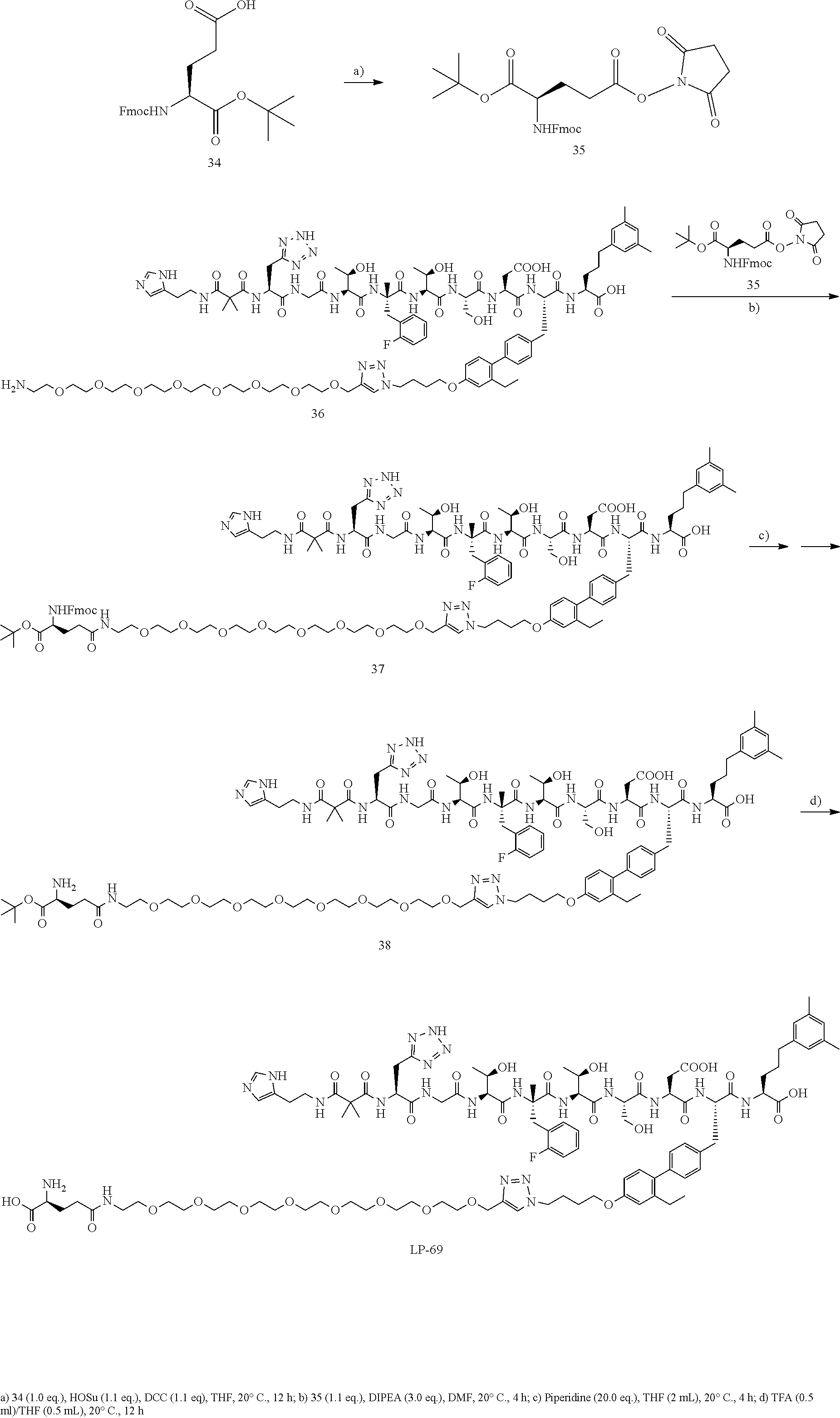

### Step 1: Synthesis of O1-tert-Butyl 05-(2,5-dioxopyrrolidin-1-yl) (2S)-2-(9H-fluoren-9-ylmethoxycarbonylamino)pentanedioate (35)

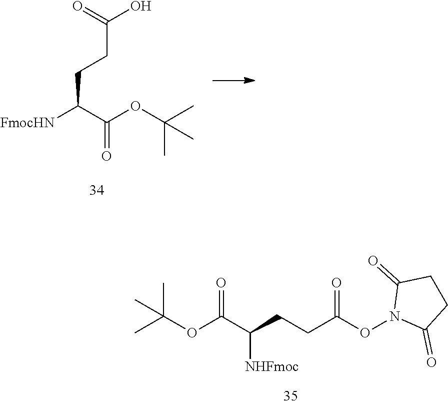

To a solution of compound 34 (2.0 g, 4.70 mmol, 1 eq) in ACN (10 mL) were added HOSu (703.28 mg, 6.11 mmol, 1.3 eq) and EDCI (1.35 g, 7.05 mmol, 1.5 eq). Then the solution was stirred at 20° C. for 12 hours. TLC (PE/EA=10/1) showed that the starting material was consumed completely and one new spot was detected. The reaction mixture was diluted with water (50 mL) and extracted with ethyl acetate (50 mL*2). The combined organic layers were washed with sat. NaCl (50 mL×2), dried over anhydrous sodium sulfate, filtered, and concentrated under reduced pressure. Compound 35 (2.0 g, 3.83 mmol, 81.42% yield) was obtained as a colorless oil.

¹H NMR (400 MHz, CHLOROFORM-d) δ ppm 7.80 (d, J=7.46 Hz, 2H), 7.59-7.69 (m, 2H), 7.41-7.47 (m, 2H), 7.32-7.39 (m, 2H), 5.52 (br d, J=7.95 Hz, 1H), 4.45-4.55 (m, 1H), 4.32-4.45 (m, 2H), 4.21-4.30 (m, 1H), 2.86 (s, 4H), 2.60-2.82 (m, 2H), 2.35 (br d, J=5.62 Hz, 1H), 2.08-2.19 (m, 1H), 1.51 (s, 9H) ppm.

### Step 2: Synthesis of (2S)-2-[[(2S)-3-[4-[4-[4-[4-[2-[2-[2-[2-[2-[2-[2-[2-[[(4S)-5-tert-Butoxy-4-(9H-fluoren-9-ylmethoxycarbonylamino)-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]propanoyl]amino]-5-(3,5-dimethylphenyl)pentanoic Acid (37) (SEQ ID NO: 344) (SEQ ID NOS 119 and 344 Disclosed Below, Respectively, in Order of Appearance)

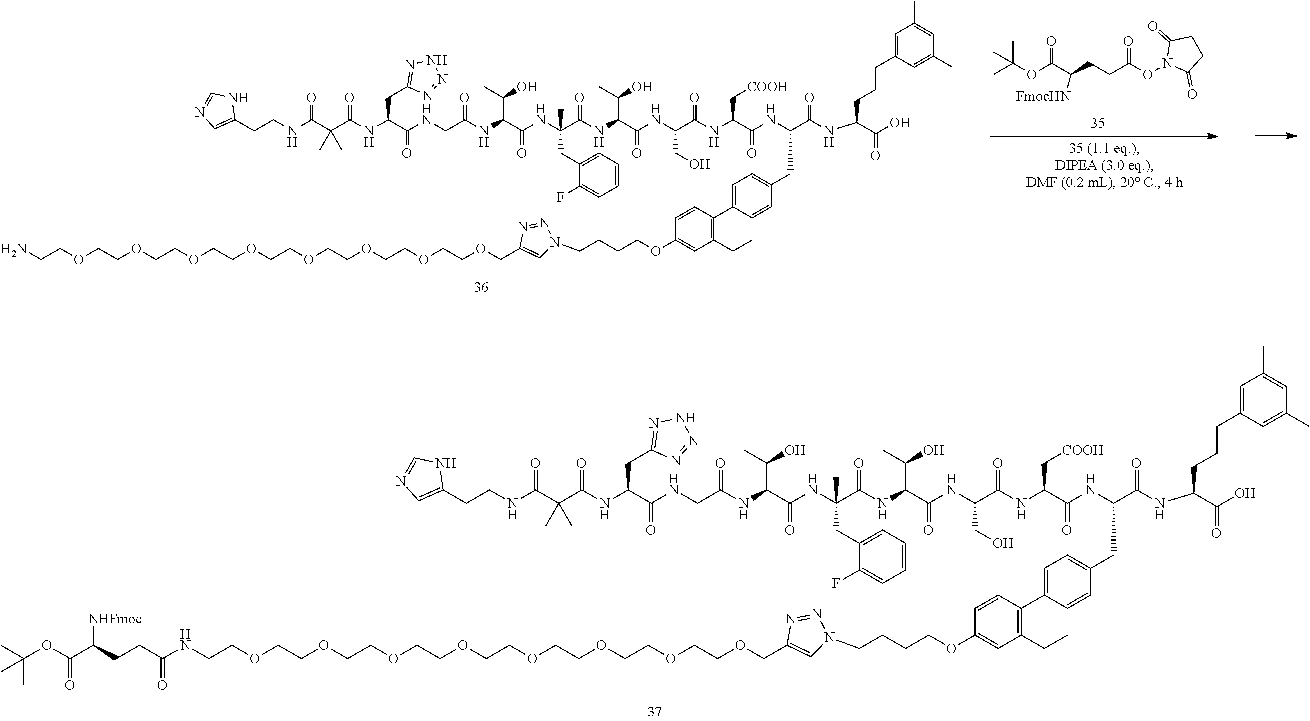

To a solution of compound 36 (60 mg, 30.30 mol, 1 eq.) and compound 35 (19.00 mg, 36.36 mol, 1.2 eq.) in DMF (2 mL) was added DIPEA (7.83 mg, 60.60 mol, 10.56 L, 2.0 eq.). Then the reaction solution was stirred at 20° C. for 4 hours. LCMS trace showed that the desired MS was detected. The solvent was removed under reduce pressure. Compound 37 (72 mg, crude) was obtained as a white solid which was used directly in the next step.

LCMS (ESI): RT=5.083 min, mass calcd. for C₁₁₈H₁₆₀FN₂₁O₃₁, 1194.08 [M+2H]²⁺, m/z found 1194.80 [M+2H]²⁺. LC-MS Conditions: Mobile Phase: 1.5 ML/4 L TFA in water (solvent A) and 0.75 ML/4 L TFA in acetonitrile (solvent B), using the elution gradient 10%-80% (solvent B) over 6.0 minutes and holding at 80% for 0.5 minutes at a flow rate of 0.8 ml/min. Column: Xtimate3 um, C18, 2.1*30 mm. Wave length: UV 220 nm and 254 nm. Column temperature: 50° C.

### Step 3: Synthesis of (2S)-2-[[(2S)-3-[4-[4-[4-[4-[2-[2-[2-[2-[2-[2-[2-[2-[[(4S)-4-Amino-5-tert-butoxy-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]propanoyl]amino]-5-(3,5-dimethylphenyl) pentanoic Acid (38) (SEQ ID NO: 345) (SEQ ID NOS 344-345 Disclosed Below, Respectively in Order of Appearance)

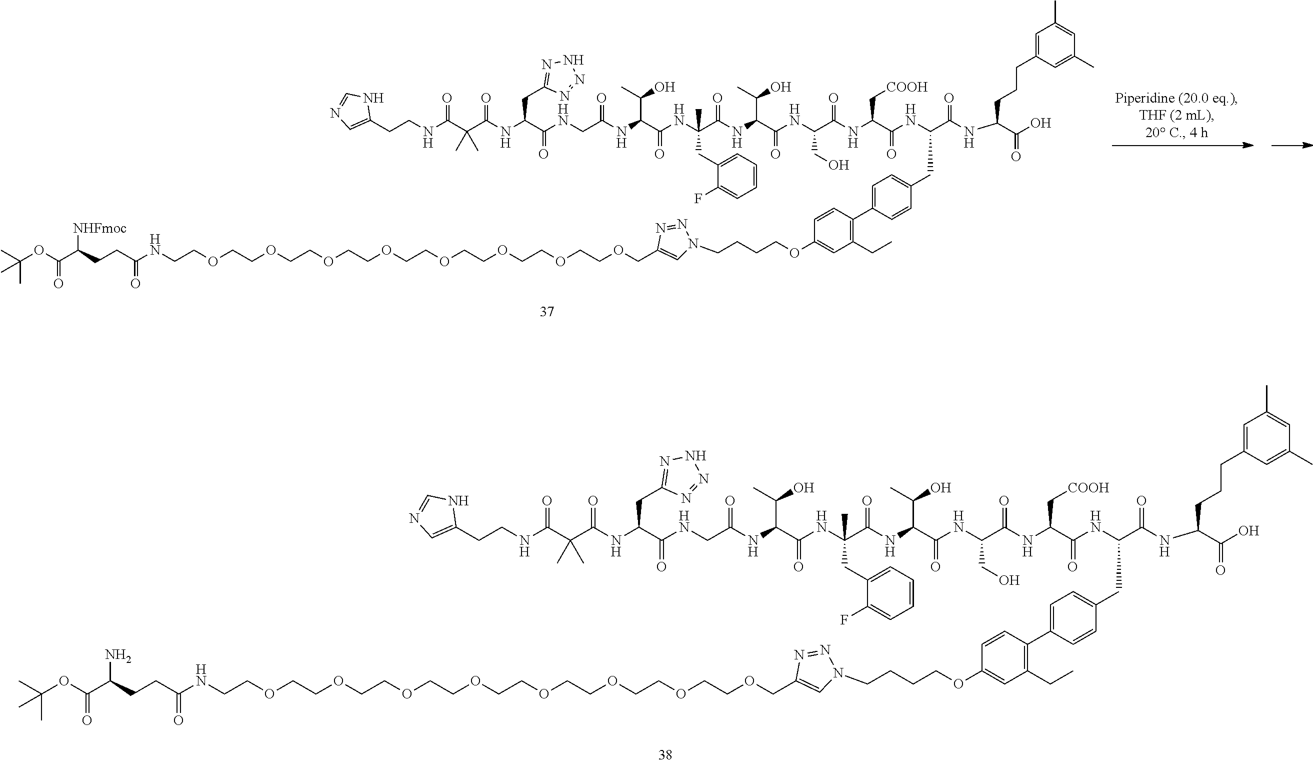

To a solution of compound 37 (72 mg, 30.16 mol, 1 eq) in THE (5 mL) was added piperidine (431.10 mg, 5.06 mmol, 0.5 mL, 167.90 eq). Then the solution was stirred at 20° C. for 2 hours. LCMS trace showed that the desired MS was detected. The solvent was removed under reduced pressure. The crude was triturated with MTBE to give the product. Compound 38 (65 mg, 30.02 mol, 99.54% yield) was obtained as a white solid which was used directly in the next step.

LCMS (ESI): RT=5.083 min, mass calcd. for C₁₀₃H₁₅₀FN₂₁O₂₉, 1083.04 [M+2H]²⁺, m/z found 1083.50 [M+2H]²⁺. LC-MS Conditions: Mobile Phase: 1.5 ML/4 L TFA in water (solvent A) and 0.75 ML/4 L TFA in acetonitrile (solvent B), using the elution gradient 10%-80% (solvent B) over 6.0 minutes and holding at 80% for 0.5 minutes at a flow rate of 0.8 ml/min. Column: Xtimate3 um, C18, 2.1*30 mm. Wave length: UV 220 nm and 254 nm. Column temperature: 50° C.

### Step 4: Synthesis of (2S)-2-[[(2S)-3-[4-[4-[4-[4-[2-[2-[2-[2-[2-[2-[2-[2-[[(4S)-4-Amino-5-tert-butoxy-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]propanoyl]amino]-5-(3,5-dimethylphenyl) pentanoic Acid (LP69) (SEQ ID NO: 120) (SEQ ID NOS 345 and 120 Disclosed Below, Respectively, in Order of Appearance)

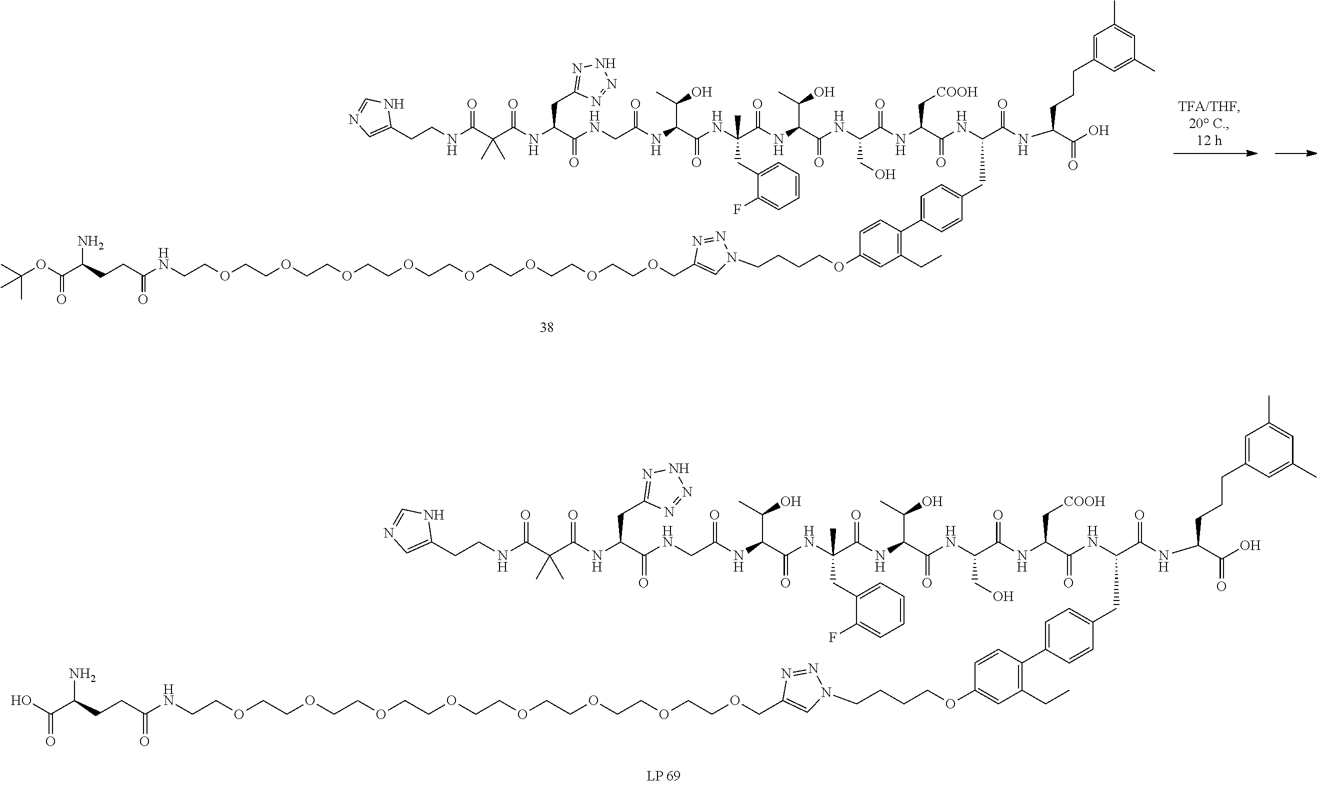

To a solution of compound 38 (65 mg, 30.02 mol, 1 eq.) in DCM (5 mL) were added TFA (7.68 g, 67.31 mmol, 5 mL, 2242.40 eq.) and TIPS (33.90 mg, 300.17 mol, 10.0 eq.). Then the solution was stirred at 20° C. for 12 hours. LCMS trace showed that the desired MS was detected. The solvent was removed under reduced pressure. The residue was purified by Prep-HPLC (column: Boston Prime C18 150*30 mm*5 um; mobile phase: [water (TFA)-ACN]; gradient: 27%-67% B over 9 min). Compound LP69 (20 mg, 8.96 mol, 29.86% yield, 97.03% purity) was obtained as a white solid. 8.2 Purification and Identification of LP69

LCMS (ESI): RT=3.516 min, m/z calcd. for C₉₉H₁₄₄FN₂₁O₂₉, 1055.02 [M+2H]²⁺, m/z found 1055.60. Mobile Phase: 1.5 mL/4 L TFA in water (solvent A) and 0.75 mL/4 L TFA in acetonitrile (solvent B) using the elution gradient 5%-95% (solvent B) over 0.7 minutes and holding at 95% for 0.4 minutes at a flow rate of 1.5 mL/min. Column: Agilent Pursult 5 C18 20*2.0 mm. Wavelength: UV 220 nm and 254 nm. Column temperature: 50° C.; MS ionization: ESI 50° C.

HPLC RT=3.343 min, 97.03% purity. HPLC method A: 1.5ML/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Example 9. Synthesis of Compound 41 (AA10-AA9-AA8-AA7-AA6-AA5-AA4-AA3-(LLQ-L)AA2-AA1-OH) (SEQ ID NO: 143 Disclosed Below)

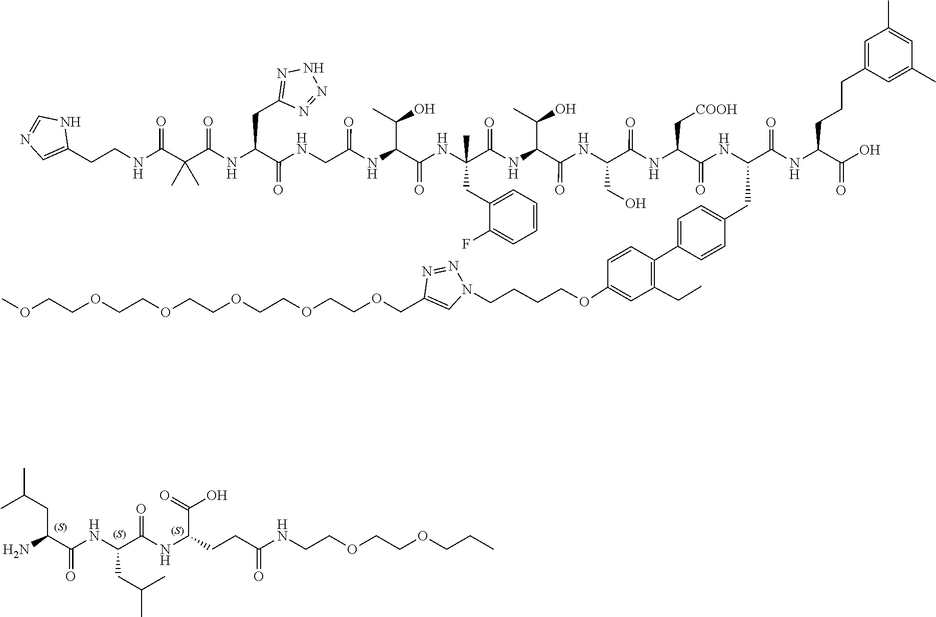

### 9.1 Synthesis, Cleavage, and Impurity Analysis of Compound 41 (SEQ ID NOS 119, 346, and 143 Disclosed Below, Respectively, in Order of Appearance)

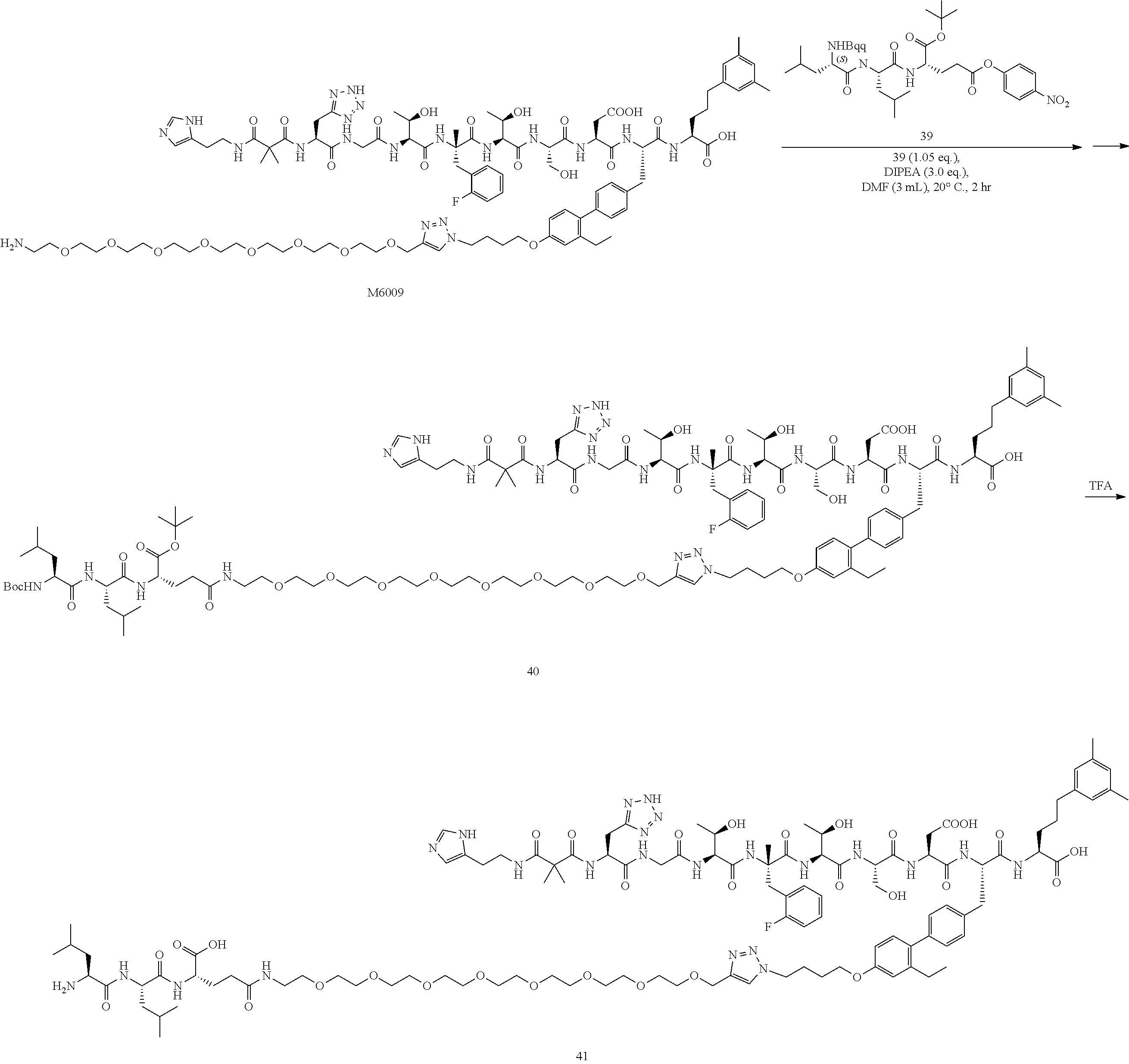

### Step 1: Synthesis of(2S)-2-[[(2S)-3-[4-[4-[4-[4-[2-[2-[2-[2-[2-[2-[2-[2-[[(4S)-5-tert-Butoxy-4-[[(2S)-2-[[(2S)-2-(tert-butoxycarbonylamino)-4-methyl-pentanoyl]amino]-4-methyl-pentanoyl]amino]-5-oxo-pentanoyl]amino]ethoxy]ethoxy]ethoxy]ethoxy]ethoxy]ethoxy]ethoxy]ethoxymethyl]triazol-1-yl]butoxy]-2-ethyl-phenyl]phenyl]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]propanoyl]amino]-5-(3,5-dimethylphenyl) pentanoic Acid (40) (SEQ ID NO: 346) (SEQ ID NOS 119 and 346 Disclosed Below, Respectively, in Order of Appearance)

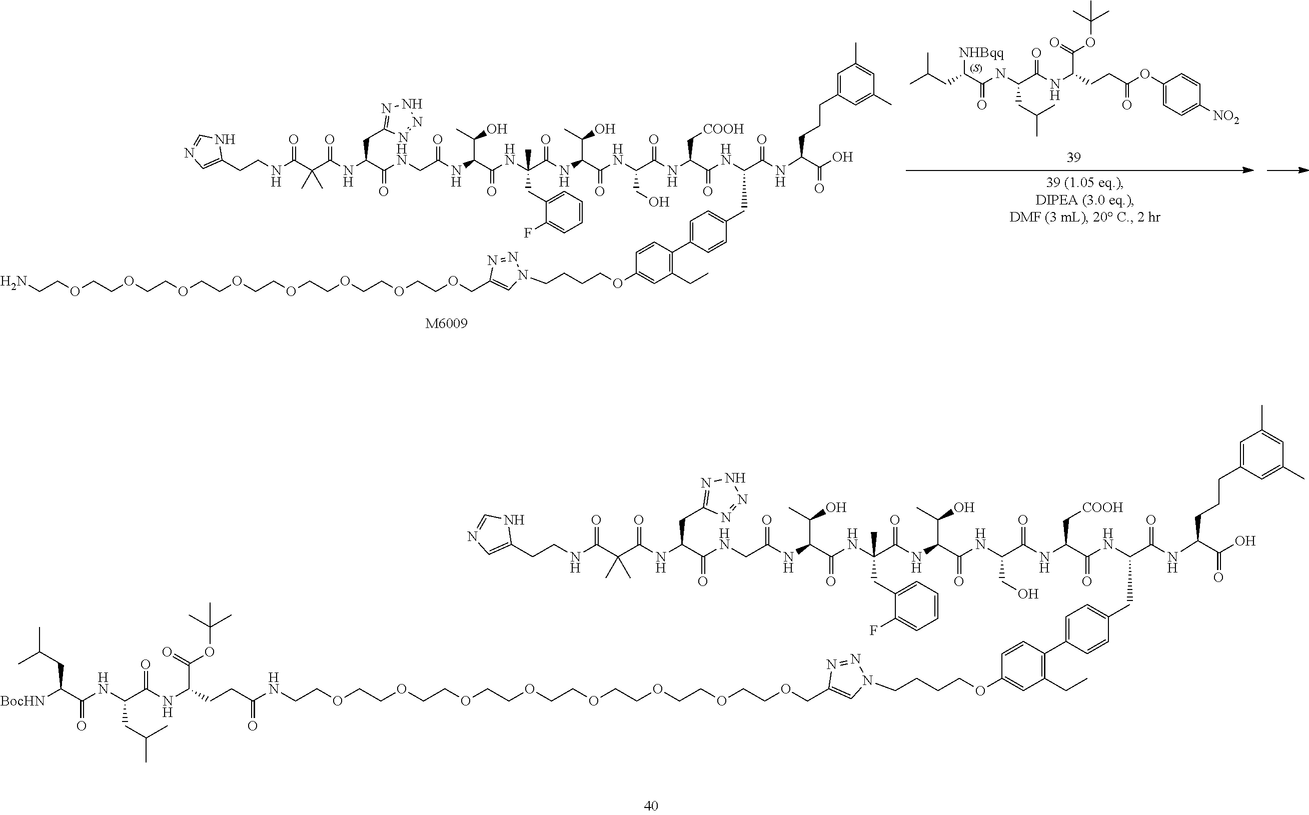

To a solution of compound M6009 (300.15 mg, 151.58 mol, 1 eq.) in DMF (5 mL) were added compound 39 (108.50 mg, 166.73 mol, 1.1 eq.) and DIPEA (39.18 mg, 303.15 mol, 52.80 μL, 2.0 eq.). Then the solution was stirred at 20° C. for 12 hours. LCMS trace showed that the desired MS was detected. The solvent was removed under reduced pressure. The residue was triturated with MTBE (20 mL*3) and filtered to give the product. Compound 40 (377 mg, crude) was obtained as a white solid which was used directly in the next step.

LCMS (ESI): RT=4.839 min, m/z calcd. for C₁₂₀H₁₈₂FN₂₃O₃₃, 1245.16 [M+2H]²⁺, m/z found 1246.60 [M+2H]²⁺, 831.10 [M+3H]³, Mobile Phase: 1.5 mL/4 L TFA in water (solvent A) and acetonitrile (solvent B), using the elution gradient 10%-80% (solvent B) over 6 minutes and holding at 80% for 2 minutes at a flow rate of 1.2 ml/min. Column: Ultimate XB-C18 3*50 mm 3 um. Wave length: UV 220 nm, 215 nm, 254 nm. Column temperature: 50° C.

### Step 2: Synthesis of (2S)-2-[[(2S)-2-[[(2S)-2-Amino-4-methyl-pentanoyl]amino]-4-methyl-pentanoyl]amino]-5-[2-[2-[2-[2-[2-[2-[2-[2-[[ ]-[4-[4-[4-[(2S)-3-[[(1S)-1-carboxy-4-(3,5-dimethylphenyl)butyl]amino]-2-[[(2S)-3-carboxy-2-[[(2S)-2-[[(2S,3R)-2-[[3-(2-fluorophenyl)-2-[[(2S,3R)-3-hydroxy-2-[[2-[[(2S)-2-[[3-[2-(]H-imidazol-5-yl)ethylamino]-2,2-dimethyl-3-oxo-propanoyl]amino]-3-(2H-tetrazol-5-yl)propanoyl]amino]acetyl]amino]butanoyl]amino]-2-methyl-propanoyl]amino]-3-hydroxy-butanoyl]amino]-3-hydroxy-propanoyl]amino]propanoyl]amino]-3-oxo-propyl]phenyl]-3-ethyl-phenoxy]butyl]triazol-4-yl]methoxy]ethoxy]ethoxy]ethoxy]ethoxy]ethoxy]ethoxy]ethoxy]ethylamino]-5-oxo-pentanoic Acid (41) (SEQ ID NO: 143) (SEQ ID NOS 346 and 143 Disclosed Below, Respectively, in Order of Appearance)

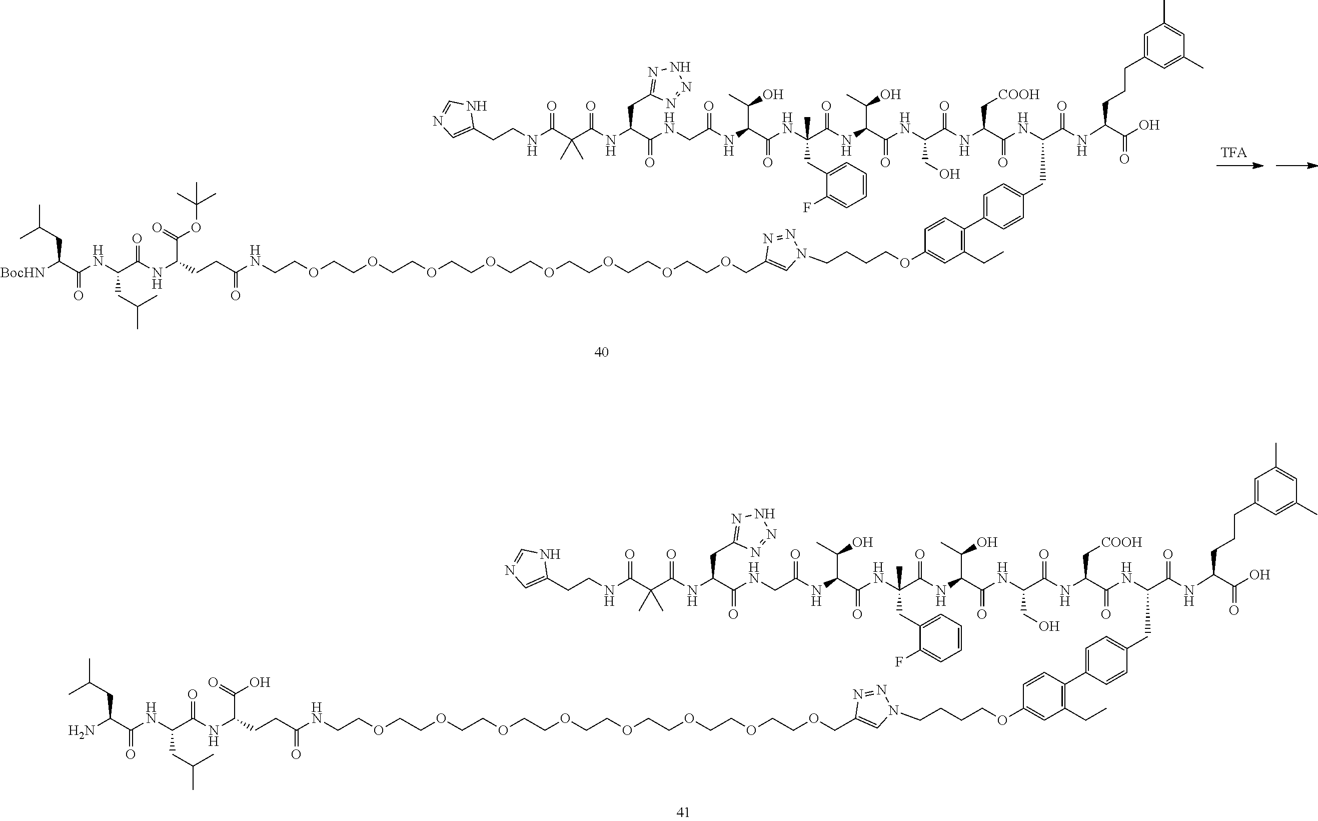

A solution of compound 40 (370.15 mg, 148.54 mol, 1 eq) in DCM (5 mL) and TFA (5 mL) was stirred at 25° C. for 4 hours. LCMS trace showed that the desired MS was detected. The solvent was removed under reduced pressure. The residue was purified by prep-HPLC (column: C18 150×30 mm; mobile phase: [water (TFA)-ACN]; gradient: 20%-60% B over 9 min). Compound 41 (51 mg, 21.20 mol, 30.95% yield, 97.10% purity) was obtained as a white solid. 9.2 Purification and Characterization of Compound 41

LCMS (ESI): RT=3.619 min, m/z calcd. for C₁₁₁H₁₆₇FN₂₄O₃₀, 1167.55 [M+2H]²⁺; m/z found 1168.60 [M+2H]²⁺, 779.40 [M+3H]³⁺. Mobile Phase: 1.5 mL/4 L TFA in water (solvent A) and 0.75 mL/4 L TFA in acetonitrile (solvent B) using the elution gradient 5%-95% (solvent B) over 0.7 minutes and holding at 95% for 0.4 minutes at a flow rate of 1.5 mL/min. Column: Agilent Pursult 5 C18 20*2.0 mm. Wavelength: UV 220 nm and 254 nm. Column temperature: 50° C. MS ionization: ESI 50° C.

HPLC RT=7.652 min, 98.63% purity. HPLC method A: Column: YMC-Pack ODS-A 150*4.6 mm, Sum; 2.75ML/4 L TFA in water (solvent A) and 2.5 ML/4 L TFA in acetonitrile (solvent B), using the elution gradient 10%-80% (solvent B) over 10 minutes and holding at 80% for 5 minutes at a flow rate of 1.5 ml/min.

### Example 10. Chemical and Physical Properties of the Peptides

Chemical and physical properties of the peptides are shown in Table 10.

TABLE 10
List of Chemical and Physical Properties

| | | | | | | GLP-1R CAMP | |
|---|---|---|---|---|---|---|---|
| | | | MS | | | EC₅₀ | Max |
| Name | WF | MW | M + H | HPLC | Amt | (nM) | (%) |
| AA10-AA9-AA8-OH | C₁₆H₂₃N₉O₅ | 421.42 | 422.1 | 96.34% | 44.7 | >1000 | −1.39 |
| | | | [M + H]⁺ | | | | |
| AA10-AA9-AA8-AA7- | C₂₀H₃₀N₁₀O₇ | 522.52 | 523.2 | 99.76% | 19.2 | >1000 | −0.52 |
| OH | | | [M + H]⁺ | | | | |
| AA10-AA9-AA8-AA7- | C₃₀H₄₀FN₁₁O₈ | 701.72 | 702.5 | 98.14% | 30.9 | >1000 | −1.13 |
| AA6-OH | | | [M + H]⁺ | | | | |
| AA10-AA9-AA8-AA7- | C₃₄H₄₇FN₁₂O₁₀ | 802.82 | 803.5 | 98.76% | 28.2 | >1000 | −1.23 |
| AA6-AA5-OH | | | [M + H]⁺ | | | | |
| AA10-AA9-AA8-AA7- | C₃₇H₅₂FN₁₃O₁₂ | 889.9 | 890.5 | 99.18% | 90 | >1000 | −0.86 |
| AA6-AA5-AA4-OH | | | [M + H]⁺ | | | | |
| AA10-AA9-AA8-AA7- | C₄₁H₅₇FN₁₄O₁₅ | 1004.99 | 1005.6 | 99.04% | 90 | >1000 | −0.25 |
| AA6-AA5-AA4-AA3- | | | [M + H]⁺ | | | | |
| OH | | | | | | | |
| AA10-AA9-AA8-AA7- | C₆₂H₈₁FN₁₈O₁₇ | 1369.44 | 1369.9 | 96.90% | 9.16 | 51.25 | 97.92 |
| AA6-AA5-AA4-AA3- | | | [M + H]⁺ | | | | |
| AA2-OH | | | | | | | |
| AA10-AA9-AA8-AA7- | C₈₁H₁₁₉FN₂₀O₂₄.2TFA | 2003.98 | 1774.87 | 98.85% | 59.8 | 59.6 | 104.11 |
| AA6-AA5-AA4-AA3- | | | | | | | |
| (L)AA2-NH2 | | | | | | | |
| AA10-AA9-AA8-AA7- | C₈₁H₁₁₈FN₁₉O₂₅ | 1776.94 | 889.4 | 99.08% | 10.04 | 882.1 | 53.33 |
| AA6-AA5-AA4-AA3- | | | [M + H]²⁺ | | | | |
| (L)AA2-OH | | | | | | | |
| AA10-AA9-AA8-AA7- | C₈₆H₁₂₅FN₂₀O₂₈ | 1906.06 | 954.0 | 97.24% | 8.54 | >1000 | 29.24 |
| AA6-AA5-AA4-AA3- | | | [M+2H]²⁺ | | | | |
| (Q-L)AA2-OH | | | | | | | |
| AA10-AA9-AA8-AA7- | C₉₈H₁₄₇FN₂₂O₃₀ | 2132.38 | 1067.2 | 95.15% | 6.02 | >1000 | 33.86 |
| AA6-AA5-AA4-AA3- | | | [M+2H]²⁺ | | | | |
| (LLQ-L)AA2-OH | | | | | | | |
| AA10-AA9-AA8-AA7- | C₇₅H₉₈FN₁₉O₁₈ | 1571.73 | 1571.73 | 98.70% | 13.89 | 2.937 | 122.44 |
| AA6-AA5-AA4-AA3- | | | | | | | |
| AA2-AA1-OH | | | | | | | |
| AA10-AA9-AA8-AA7- | C₉₄H₁₃₅FN₂₀O₂₆.2TFA | 2208.24 | 990.9 | 98.81% | 10 | 2.234 | 139.7 |
| AA6-AA5-AA4-AA3- | | | [M+2H]²⁺ | | | | |
| (L)AA2-AA1-OH | | | | | | | |
| AA10-AA9-AA8-AA7- | C₉₉H₁₄₂FN₂₁O₂₉.2TFA | 2337.39 | 1055.02 | 97.03% | 4.19 | 3.215 | 106.2 |
| AA6-AA5-AA4-AA3- | | | [M+2H]²⁺ | | | | |
| (Q-L)AA2-AA1-OH | | | | | | | |
| AA10-AA9-AA8-AA7- | C₁₁₁H₁₆₄FN₂₃O₃₁ | 2335.66 | 1168.8 | 97.10% | 11.1 | 13.4 | 103.80 |
| AA6-AA5-AA4-AA3- | | | [M+2H]²⁺ | | | | |
| (LLQ-L)AA2-AA1-OH | | | | | | | |

### Example 11. Metabolite Study of M3190 and its Derivatives, and Discovery of Peptide Acid Compounds as a GLP1R Agonist

Metabolite studies were conducted using Q-M3190 and Q-6009 (Q is Glutamine). GLP1R agonist (Q-M3190, Q-M6009), together with GLP1R-ATL REGN18121-M3190, were evaluated in human liver S9 with/without NADPH and UDPGA (FIG. 6).

### 11.1 Metabolism Studies in Hepatocytes Vs. S9 vs. Microsomes

Hepatocytes are complete liver cells, containing various first-phase and second-phase enzymes that can mediate various metabolic reactions; therefore, it is a better in vitro model to test metabolites.

Liver S9 is the supernatant obtained by grinding and centrifuging the liver cells. The enzymes content is lower compared to Hepatocytes. It mainly contains CYP enzymes and some biphasic enzymes (but no biphasic coenzyme), so additional coenzyme (NADPH and UDPGA, etc.) is needed to mediate the biphasic reaction.

Liver microsomes are the lower part obtained by grinding and centrifuging the liver cells and mainly mediate a phase reaction. Because the cell membrane is destroyed, the test-compound has no limitation to pass through the cell membrane and is directly exposed to the liver enzymes that are also in liver microsome and S9.

Major phase I enzymes are mainly cytochrome P450 system, which is a family of membrane-bound enzymes found within the endoplasmic reticulum of hepatocytes.

Major phase II enzymes are UDP-glucuronosyltransferases, sulfotransferases, N-acetyltransferases, glutathione S-transferases, and methyltransferases (mainly thiopurine S-methyl transferase and catechol O-methyl transferase).

High resolution LC-MS was used to analyze the metabolized products and the results are shown in the Tables 11 and 12 below.

### 11.2 Metabolites of Q-M3190 in Human Liver S9 (SEQ TD NO: 347 Disclosed Below)

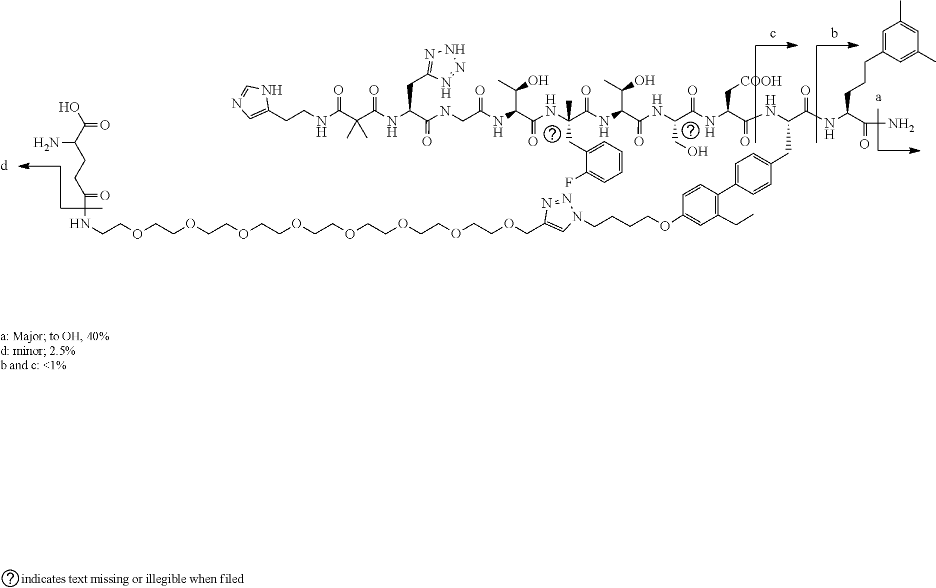

TABLE 11
Metabolites of Q-M3190

| | | | | | Relative |
|---|---|---|---|---|---|
| Metabolite | Metabolites at 4 hours | [M + | Charge | RT | Abundance |
| Code | in human liver S9 | nH]ⁿ⁺ | (n) | (min) | (% Total) |
| M1 | AA10-AA(-AA8-AA7-AA6- | 1004.41 | 1 | 5.03 | + |
| | AA5-AA4-AA3-OH | | | | |
| M2 | (Q-L)AA2-OH | 918.50 | 2 | 7.88 | + |
| M3 | (Q-L)AA2-AA1-NH2 | 1120.64 | 2 | 10.43 | + |
| M4 | (L)AA2-AA1-OH | 992.58 | 2 | 10.56 | + |
| M5 | (Q-L)AA2-AA1-OH | 1121.63 | 2 | 10.89 | + |
| M6 | M3190 | 1978.00 | 2 | 11.50 | 2.76 |
| M7 | M3 190 N-adduct | 2248.16* | 3 | 11.58 | + |
| 283 | Q-M3190 | 2107.04 | 3 | 11.69 | 56.51 |
| M8 | M6009 | 1978.98* | 2 | 11.77 | 2.34 |
| M9 | M6009 N-adduct | 2249.14 | 3 | 11.86 | + |
| M10 | Q-M6009 | 2108.03 | 3 | 11.98 | 38.40 |

### 11.3 Metabolites of Q-M6009 in Human Liver S9 (SEQ ID NO: 354 Disclosed Below)

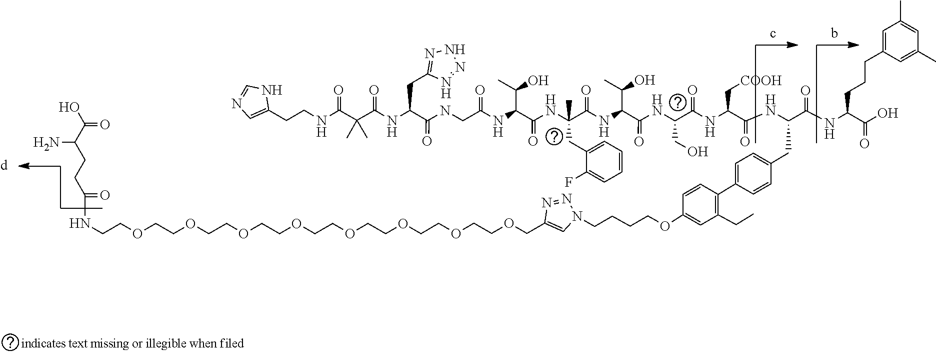

Similarly to M3190, Major cleavage sites are on d for lost Q, b and c for lost AA1 and AA10-AA3. The main remaining is M6009 in 93%. 1% (Q-L) AA2 and 50 (Q-L) AA2-AA1 were detected.

TABLE 12
Metabolites of Q-M6009

| | | | | | Relative |
|---|---|---|---|---|---|
| Metabolite | Metabolites at 4 hours | [M + | Charge | RT | Abundance |
| Code | in human liver S9 | nH]ⁿ⁺ | (n) | (min) | (% Total) |
| M1 | AA10-AA(-AA8-AA7- | 1005.4197 | 1 | 4.86 | 0.21 |
| | AA6-AA5-AA4-AA3 | | | | |
| M2 | AA2 | 790.4604 | 1 | 7.30 | 0.14 |
| M3 | (Q-L)AA2 | 919.5031 | 1 | 7.30 | 0.95 |
| M4 | (Q-L)AA2-AA1 | 561.8210 | 2 | 10.11 | 4.87 |
| M5 | (L)AA2-AA1 | 497.2996 | 2 | 10.11 | 0.38 |
| M6 | AA4-AA3-(L)AA2-AA1 | 662.8510 | 2 | 10.16 | 0.12 |
| M7 | AA6-AA5-AA4-AA3- | 802.9126 | 2 | 10.50 | 0.05 |
| | (Q-L)AA2-AA1 | | | | |
| M8 | M6009 | 990.4998 | 2 | 11.00 | 13.14 |
| LP69 | Q-M6009 | 1055.0206 | 2 | 11.02 | 79.84 |
| M9 | AA8-AA7-AA6-AA5-AA4- | 881.9460 | 2 | 11.10 | 0.13 |
| | AA3-(Q-L)AA2-AA1 | | | | |
| M10 | AA7-AA6-AA5-AA4- | 853.4356 | 2 | 11.16 | 0.17 |
| | AA3-(Q-L)AA2-AA1 | | | | |

Peptide C-M3190 identified from metabolite studies of M3190-antibody conjugate was converted to C-M6009 in vivo in GLP1R humanized mice.

From a tryptic digestion of the parent M3190-ATL, peptide c-M3190 is a peptide and their structures/sequences were determined by high resolution mass spectroscopy as shown below.

TABLE 13
Metabolites of c-M3190 and c-M6009

| Peptide | Metabolite |
|---|---|
| c-M3190 | LLQ(-M3190)GSGDIQMTQSPSSVSASVGDR |
| | (SEQ ID NO: 70) |
| c-M6009 | LLQ(-M6009)GSGDIQMTQSPSSVSASVGDR |
| | (SEQ ID NO: 71) |

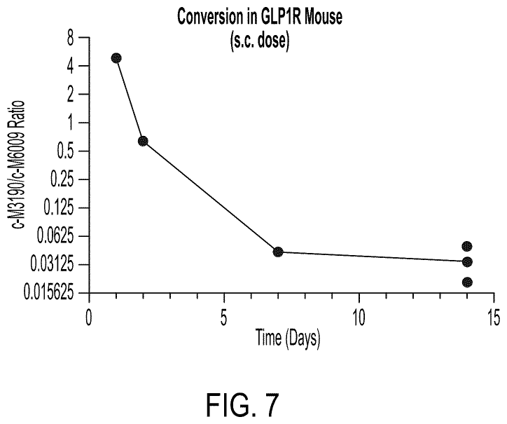

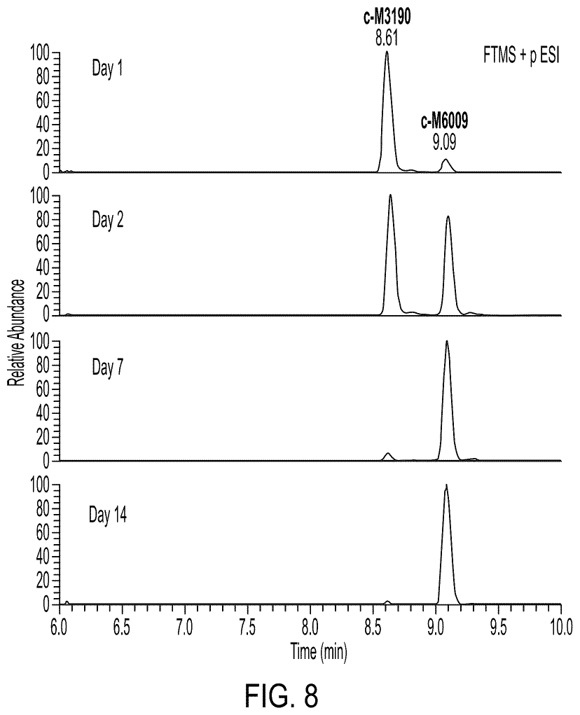

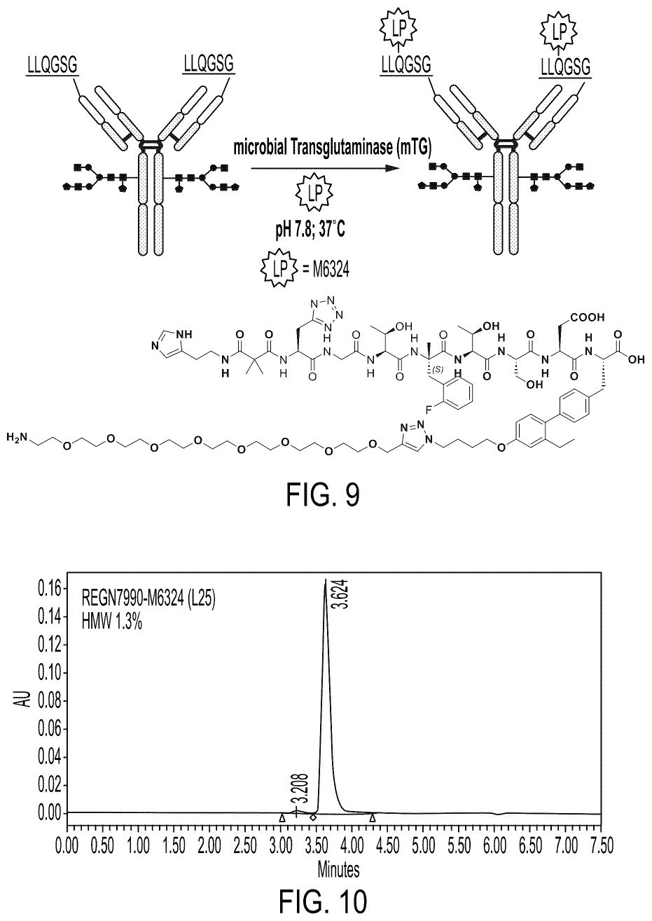

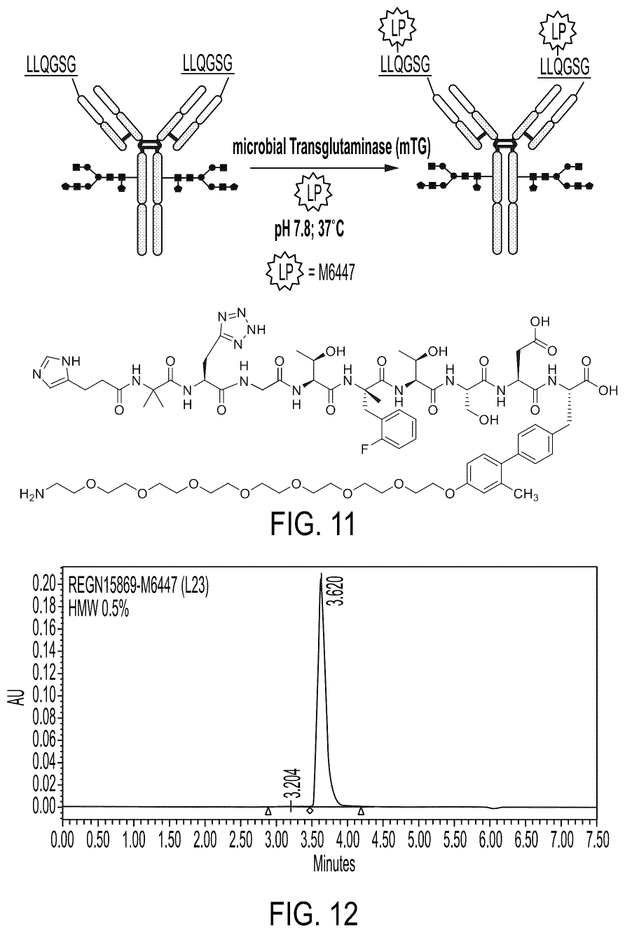

In vivo mouse s.c. dosing study showed that c-M3190 ATL was slowly converted to c-M6009 after 7 days in a GLP1R humanized mouse (FIGS. 7 and 8). 11.4 Syntheses and Characterization of Peptide Compounds Identified from M3190 Metabolite Study

TABLE 14
Peptides Identified from M3190 Metabolite Study

| | cAMP | | | | |
|---|---|---|---|---|---|
| | EC₅₀ | | | MS/ | |
| Name (peptide amide) | (nm) | M code | MW | M + H | HPLC |
| AA9-AA8-AA7-AA6-AA5-AA4- | 350.7 | M6295 | 1772.01 | 887.0 | 99.71% |
| AA3-(L)AA2-AA1-NH₂ | | | | [M + 2H]²⁺ | |
| AA9-AA8-AA7-AA6-AA5-AA4- | 280 | M2383 | 1364.48 | | |
| AA3-(N3)AA2-AA1-NH₂ | | | | | |
| AA9-AA8-AA7-AA6-AA5-AA4- | 19 | M2742 | 1566.53 | 670.0 | |
| AA3-(NH₂)AA2-AA1-NH₂ | | | | [M + 2H]2+ | |
| AA10-AA9-AA8-AA7-AA6-AA5- | 0.4975/ | M2361 | 1685.73 | 786.5 | 98.14% |
| AA4-AA3-(N₃)AA2-AA1-NH2 | 0.3587 | | | [M + 2H]²⁺ | |
| AA10-AA9-AA8-AA7-AA6-AA5- | 0.05019 | M2642 | 1773.76 | 773.8 | |
| AA4-AA3-(NH₂)AA2-AA1-NH2 | | | | [M + 2H]2+ | |
| AA10-AA9-AA8-AA7-AA6-AA5- | 0.3178 | M3190 | 2207.26 | 990.3 | 98.70% |
| AA4-AA3-(L)AA2-AA1-NH2 | | | | [M + 2H]²⁺ | |
| AA10-AA9-AA8-AA7-AA6-AA5- | 1.072 | M6067 | 2108.32 | 1054.9 | 97.90% |
| AA4-AA3-(Q-L)AA2-AA1-NH₂ | | (Q-M3190) | | [M + 2H]²⁺ | |
| AA10-AA9-AA8-AA7-AA6-AA5- | 1.188 | M6296 | 2334.68 | 1168.1 | 99.08% |
| AA4-AA3-(LLQ-L)AA2-AA1-NH₂ | | (LLQ- | | [M + 2H]²⁺ | |
| | | M3190) | | 779.0 | |
| | | | | [M + 3H]³⁺ | |
| AA10-AA9-AA8-AA7-AA6-AA5- | 1.887 | | 2155.42 | | 95.9% |
| AA4-AA3-(PEG12)AA2-AA1-NH2 | | | | | |

TABLE 15
Peptides Identified from M3190 Metabolite Study

| | cAMP | | | | |
|---|---|---|---|---|---|
| | EC₅₀ | | | MS/ | |
| Name (peptide acids) | (nm) | M code | MW | M + H | HPLC |
| AA10-AA9-AA8-AA7-AA6-AA5- | 51.25 | M6320 | 1369.44 | 1369.9 | 96.90% |
| AA4-AA3-AA2-OH | | | | [M + H]⁺ | |
| AA10-AA9-AA8-AA7-AA6-AA5- | 59.6 | | 2003.98 | 1774.87 | 98.85% |
| AA4-AA3-(L)AA2-NH2 | | | | | |
| AA10-AA9-AA8-AA7-AA6-AA5- | 882.1 | M6324 | 1776.94 | 889.4 | 99.08% |
| AA4-AA3-(L)AA2-OH | | | | [M + H]²⁺ | |
| AA10-AA9-AA8-AA7-AA6-AA5- | >1000 | M6322 | 1906.06 | 954.0 | 97.24% |
| AA4-AA3-(Q-L)AA2-OH | | | | [M + 2H]²⁺ | |
| AA10-AA9-AA8-AA7-AA6-AA5- | >1000 | M6321 | 2132.38 | 1067.2 | 95.15% |
| AA4-AA3-(LLQ-L)AA2-OH | | | | [M + 2H]²⁺ | |
| AA10-AA9-AA8-AA7-AA6-AA5- | 2.937 | payload | 1571.73 | 1571.73 | 98.70% |
| AA4-AA3-AA2-AA1-OH | | | | | |
| AA10-AA9-AA8-AA7-AA6-AA5- | 2.234 | M6009 | 2208.24 | 990.9 | 98.81% |
| AA4-AA3-(L)AA2-AA1-OH | | | | [M + 2H]²⁺ | |
| AA10-AA9-AA8-AA7-AA6-AA5- | 3.215 | Q-M6009 | 2337.39 | 1055.02 | 97.03% |
| AA4-AA3-(Q-L)AA2-AA1-OH | | | | [M + 2H]²⁺ | |
| AA10-AA9-AA8-AA7-AA6-AA5- | 13.4 | M6297 | 2335.66 | 1168.8 | 97.10% |
| AA4-AA3-(LLQ-L)AA2-AA1-OH | | (LLQ- | | [M + 2H]²⁺ | |
| | | M6009) | | | |

### Example 12. Preparation of a New Fmoc-AA2 Building Block (M6508/Cpd #542)

### Synthesis of M6508/Cpd #542

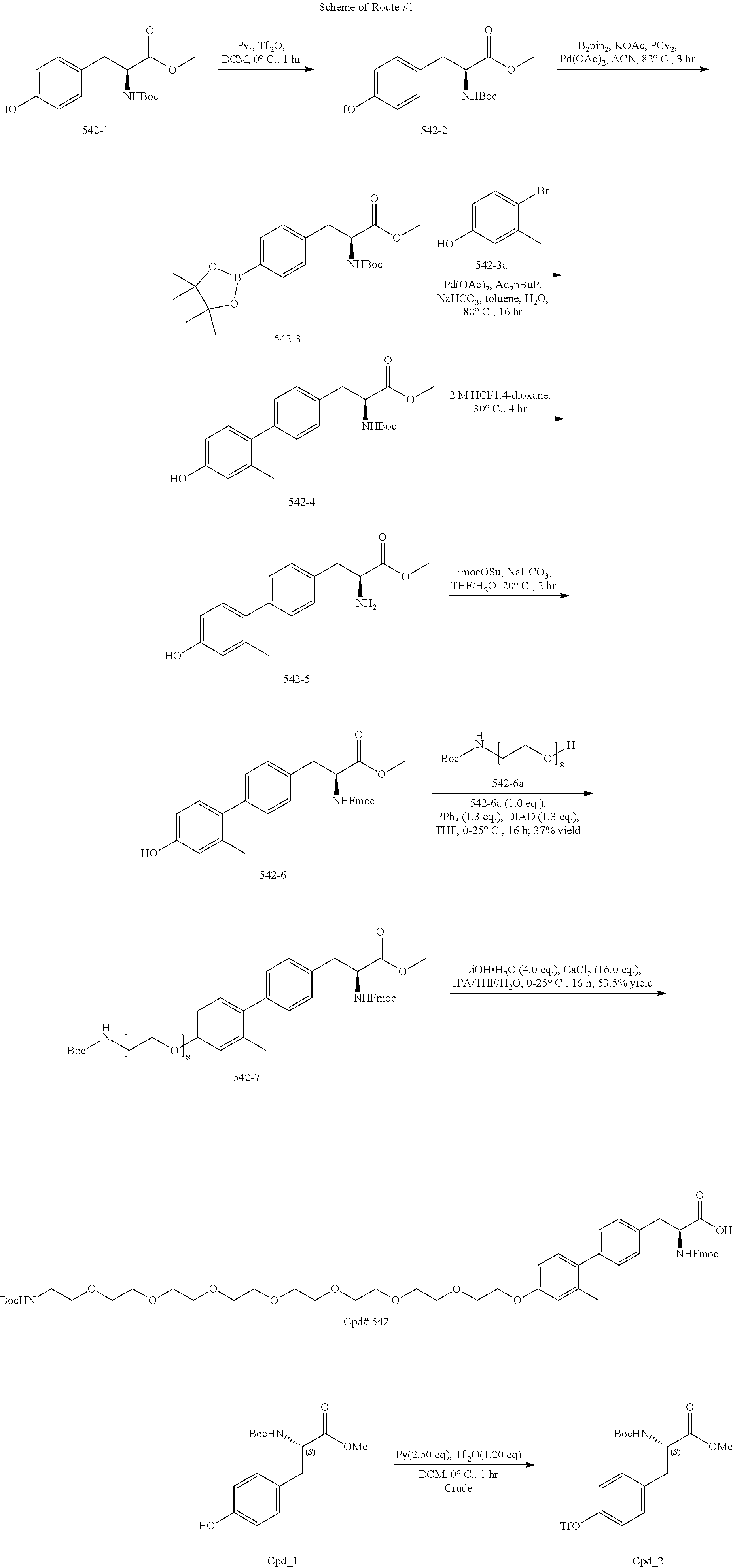

A general procedure is as follows (Batch 1′-1):
1) Reaction set up: To a solution of Cpd_1 (160 g, 541 mmol, 1.00 eq) in DCM (900 mL) was added Tf₂O (183 g, 650 mmol, 107 mL, 1.20 eq) at 0° C., then Py (107 g, 1.35 mol, 109 mL, 2.50 eq) was added dropwise at 0° C. under N₂. The mixture was stirred at 0° C. for 1 hr.
2) Monitoring: TLC (Petroleum ether: Ethyl acetate=3:1, Plate 1) indicated Cpd_1 (R_{f}=0.10) was consumed completely, and a new spot (R_{f}=0.35) formed.
3) Work up: The reaction mixture was quenched by adding H₂O (900 mL) at 0° C. The mixture was adjusted pH to 2-3 with saturated citric acid solution. After separation, the organic layer was washed with 5% citric acid solution (500 mL) and H₂O (500 mL*3), dried over Na₂SO₄, filtered, and concentrated under reduced pressure to give the residue (255 g, crude, EC6553-2403-Int-1).
4) Purification: The crude product was used into the next step without further purification.
5) Result: The crude product was used into the next step.

### Cpd_3 Synthesis

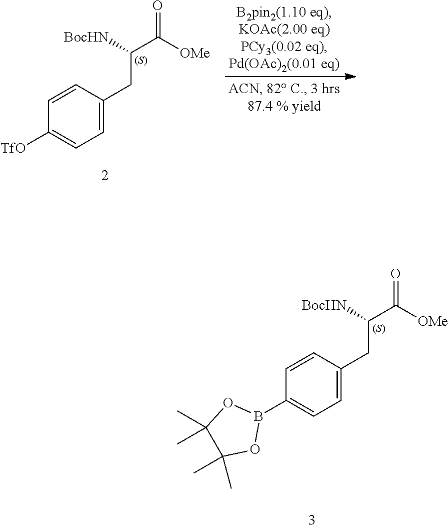

A general procedure is as follows (batch 2′-1):
1) Reaction set up: The Cpd_2 (255 g, crude, EC6553-2403-Int-1) was dissolved in ACN (1300 mL), then added B₂Pin₂ (151 g, 595 mmol, 1.10 eq), KOAc (106 g, 1.08 mol, 2.00 eq) and PCy₃ (3.04 g, 10.8 mmol, 0.02 eq). The mixture was degassed and purged with N₂ for 3 times. Then Pd(OAc)₂ (1.22 g, 5.42 mmol, 0.01 eq) was added to the mixture under N₂. The reaction mixture was heated to 82° C. for 3 hrs under N₂.
2) Monitoring: TLC (Petroleum ether: Ethyl acetate=3:1, Plate 2) indicated Cpd_2 (R_{f}=0.35) was consumed completely, and a new spot (R_{f}=0.50) formed.
3) Work up: The reaction mixture was filtered; the cake was washed with ACN (150 mL). The combined filtrate was concentrated and diluted with MTBE (1.00 L), then washed with water (400 mL*2), dried over Na₂SO₄, filtered, and concentrated under reduced pressure to give a residue.
4) Purification: The residue was used into the next step without further purification. 5) Result: Cpd_3 (202 g, 473 mmol, 87.4% yield, 95.0% purity) was obtained as yellow oil. HNMR (EC6553-2403-P1A1), LCMS (EC6553-2403-P1B1, Rt=0.50 min), HPLC (EC6553-2403-P1B2, Rt=3.32 min).

HNMR of Cpd_3-EC6553-2403-P1A1: HNMR (DMSO-d6, Bruker_E_400 MHz) δ 7.59 (d, J=7.6 Hz, 2H), 7.32-7.19 (m, 3H), 4.23-4.10 (m, 1H), 3.61 (s, 3H), 3.05-2.95 (m, 1H), 2.93-2.81 (m, 1H), 1.31 (s, 9H), 1.27 (s, 12H).

MS cal.: 405.23, MS observed: [M+Na]⁺=428.0. HPLC purity: 95.0% (220 nm)

### Cpd 5 Synthesis

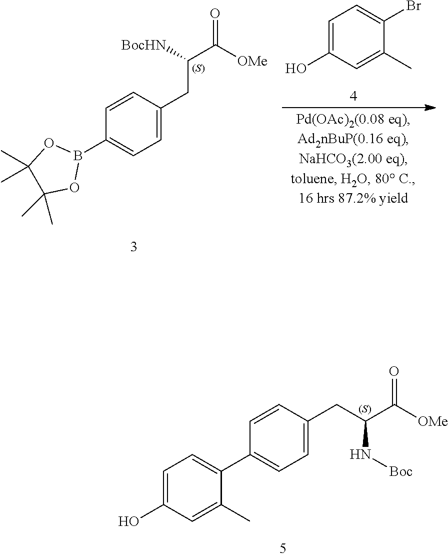

A general procedure is as follows (batch 3′-3):
1) Reaction set up: To a solution of Cpd_3 (125 g, 293 mmol, 0.95 eq) and Cpd_4 (57.6 g, 308 mmol, 1.00 eq) in toluene (600 mL) and H₂O (180 mL) were added NaHCO₃ (51.8 g, 616 mmol, 2.00 eq), Ad₂nBuP (17.6 g, 49.3 mmol, 0.16 eq) and Pd(OAc)₂ (5.54 g, 24.6 mmol, 0.08 eq) under N₂ atmosphere. The mixture was stirred at 80° C. for 16 hrs under N₂ atmosphere.
2) Monitoring: LCMS (EC6530-603-P1A1) showed Cpd_3 was consumed completely and desired mass (Rt=1.48 min) detected.
3) Work up: T The reaction mixture was diluted with H₂O (500 mL) and extracted with EtOAc (250 mL*2). The combined organic layers were washed with H₂O (100 mL) and brine (100 mL), dried over Na₂SO₄ filtered and concentrated under reduced pressure to give a residue.
4) Purification: The residue was purified by column chromatography (SiO₂, Petroleum ether: Ethyl acetate=1/O to 3/1, Petroleum ether: Ethyl acetate=3:1, R_{f}=0.30).
5) Result: Cpd_7 (117 g, 291 mmol, 87.2% yield, 95.9% purity) was obtained as yellow oil. HPLC: Rt=2.92 min, HPLC purity: 95.9% (220 nm). LCMS (Rt=1.50 min), MS cal.: 385.19, MS observed: [M+Na]⁺=408.1

HNMR (CDCl₃, Bruker_G_400 MHz) δ 7.23-7.11 (m, 4H), 7.06 (br d, J=8.0 Hz, 1H), 6.77 (d, J=2.4 Hz, 1H), 6.71 (br d, J=8.0 Hz, 1H), 5.09 (br d, J=8.4 Hz, 1H), 4.71-4.56 (m, 1H), 3.74 (s, 3H), 3.23-3.01 (m, 2H), 2.21 (s, 3H), 1.43 (s, 9H).

### Cpd_6 Synthesis

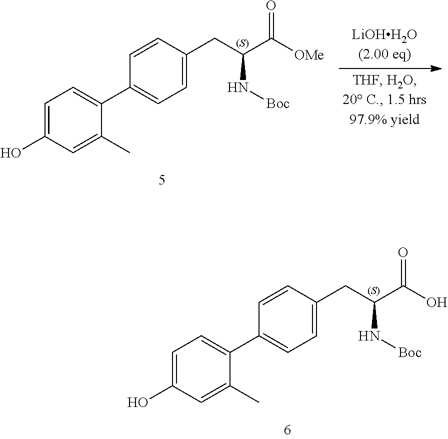

A general procedure is as follows (batch 4′-2):
1) Reaction set up: To a solution of Cpd_5 (117 g, 291 mmol, 1.00 eq) in THF (800 mL) was added a solution of LiOH·H₂O (24.4 g, 582 mmol, 2.00 eq) in H₂O (400 mL). The mixture was stirred at 20° C. for 1.5 hrs.
2) Monitoring: LCMS (EC6530-611-P1A1) showed Cpd_5 was consumed completely and desired mass (Rₜ=0.46 min) detected.
3) Work up: The reaction was added ice-water (800 mL), then added 1M HCl to adjusted pH=4˜5, then extracted with EtOAc (400 mL*2), the combined organic phase was washed with H₂O (400 mL) and brine (300 mL), dried over anhydrous Na₂SO₄, filtered and concentrated to give the crude product.
4) Purification: The crude product was used into the next step without further purification. 5) Result: Cpd_6 (110 g, 285 mmol, 97.9% yield, 96.3% purity) was obtained as yellow oil. HPLC: Rₜ=2.52 min, purity 96.3% (220 nm). LCMS(Rₜ=1.32 min), MS cal.: 371.17, MS observed: [M+Na]⁺=394.1

HNMR (CDCl₃, Bruker_G_400 MHz) δ 7.18 (s, 4H), 7.04 (br d, J=8.0 Hz, 1H), 6.74 (d, J=2.4 Hz, 1H), 6.68 (br d, J=7.2 Hz, 1H), 5.10 (br d, J=7.6 Hz, 1H), 4.73-4.42 (m, 1H), 3.29-2.89 (m, 2H), 2.18 (s, 3H), 1.49-1.33 (m, 9H)

### Cpd_7 Synthesis

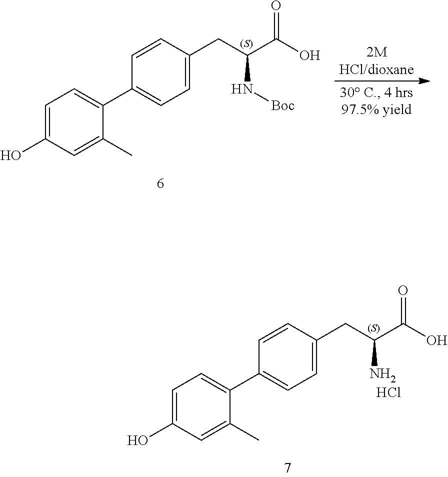

A general procedure is as follows (batch 5′-2):
1) Reaction set up: To a solution of Cpd_6 (110 g, 285 mmol, 1.00 eq) in HCl/dioxane (2 M, 491 mL). The mixture was stirred at 30° C. for 4 hrs.
2) Monitoring: LCMS (EC6530-612-P1A1) showed Cpd_6 was consumed completely and desired mass (Rₜ=0.72 min) detected.
3) Work up: The reaction was concentrated to give the crude product.
4) Purification: The crude product was triturated with Petroleum ether/MTBE (6/1, 400 mL) at 20° C. for 16 hrs.
5) Result: Cpd_7 (87.6 g, 278. mmol, 97.5% yield, 97.7% purity, HCl salt) was obtained as an off-white solid. HNMR (EC6530-612-P1A1), HPLC(Rₜ=0.94 min), HPLC purity: 97.7% (220 nm). LCMS(Rₜ=0.70 min), MS cal.: 271.12, MS observed: [M+H]=272.3

HNMR (DMSO-d6, Bruker_D_400 MHz) δ 9.48 (br s, 1H), 8.56 (br s, 3H), 7.31 (d, J=8.4 Hz, 2H), 7.22 (d, J=8.0 Hz, 2H), 6.96 (d, J=8.0 Hz, 1H), 6.78-6.62 (m, 2H), 4.16 (br s, 1H), 3.19 (br d, J=6.0 Hz, 2H), 2.15 (s, 3H).

### Cpd_8 Synthesis

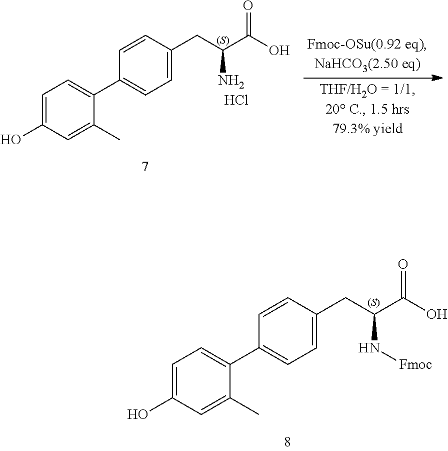

A general procedure is as follows (batch 6′-2):
1) Reaction set up: To a solution of Cpd_7 (87.6 g, 278 mmol, 1.00 eq, HCl salt) in THF (800 mL) and H₂O (800 mL) was added NaHCO₃ (58.4 g, 695 mmol, 2.50 eq) and FmocOSu (86.3 g, 255 mmol, 0.92 eq). The mixture was stirred at 20° C. for 3 hrs. 2) Monitoring: LCMS (EC6530-633-P1A1) showed Cpd_7 was consumed completely and desired mass (Rₜ=0.47 min) detected.
3) Work up: The reaction was added ice-water (1000 mL), then added 1M HCl to adjusted pH=4˜5, then extracted with EtOAc (500 mL*2), the combined organic phase was washed with H₂O (500 mL) and brine (400 mL), dried over anhydrous Na₂SO₄, filtered and concentrated to give the crude product.
4) Purification: The residue was purified by column chromatography (SiO₂, Dichloromethane: Methanol=1/0 to 8/1, Dichloromethane:Methanol=10:1, R_{f}=0.50).
5) Result: Cpd_8 (100 g, 198 mmol, 79.3% yield, 98.0% purity) was obtained as white solid.

HNMR (EC6530-633-P1A1), HPLC(Rₜ=3.17 min), HPLC purity 98.0% (220 nm). SFC(Rₜ=1.53 min, ee=100%). LCMS(Rₜ=0.50 min), MS cal.: 493.19, MS observed: [(M+Na]=516.2. e.e. value %: 100%

HNMR (CDCl₃, Bruker_H_400 MHz) δ 7.75 (br d, J=7.6 Hz, 2H), 7.61-7.48 (m, 2H), 7.39 (t, J=7.6 Hz, 2H), 7.29 (t, J=7.6 Hz, 2H), 7.20-6.92 (m, 5H), 6.71 (br s, 1H), 6.65 (br d, J=8.0 Hz, 1H), 5.34 (br d, J=8.0 Hz, 1H), 4.82-4.70 (m, 1H), 4.54-4.45 (m, 1H), 4.43-4.34 (m, 1H), 4.21 (br t, J=6.8 Hz, 1H), 3.33-3.04 (m, 2H), 2.21-2.08 (m, 3H).

### Cpd_9 Synthesis

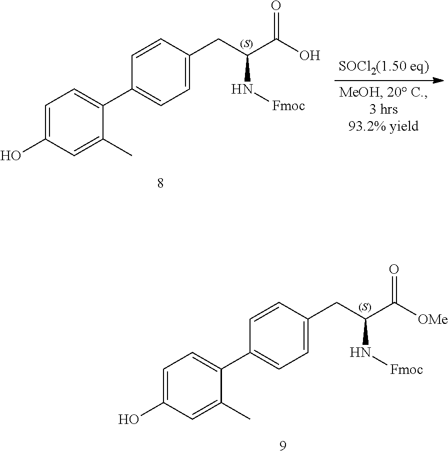

A general procedure is as follows (batch 7′-3):
1) Reaction set up: To a solution of Cpd_8 (120 g, 243 mmol, 1.00 eq) in MeOH (800 mL) was added SOCl₂ (43.3 g, 364 mmol, 26.4 mL, 1.50 eq) drop-wise at 20° C. The mixture was stirred at 20° C. for 3 hrs.
2) Monitoring: TLC (Dichloromethane:Methanol=10:1) showed Cpd_8 (R_{f}=0.40) was consumed completely and one new major spot (R_{f}=0.50) formed.
3) Work up: The reaction mixture was concentrated under reduced pressure to give a residue. Then diluted with DCM (800 mL) and washed with saturated NaHCO₃ aqueous solution (300 mL*2), H₂O (200 mL) and brine (200 mL), dried over Na₂SO₄, filtered and concentrated under reduced pressure to give the crude product.
4) Purification: The crude product was used into the next step without further purification.
5) Result: Cpd_9 (116 g, 226 mmol, 93.2% yield, 99.2% purity) was obtained as white solid. HPLC(Rₜ=3.56 min), HPLC purity: 99.2% (220 nm). LCMS(Rₜ=0.50 min), MS cal.: 507.20, MS observed: [M+H]⁺=508.2

HNMR (CDCl₃, Bruker_F_400 MHz) δ 7.77 (d, J=7.6 Hz, 2H), 7.63-7.53 (m, 2H), 7.40 (t, J=7.2 Hz, 2H), 7.36-7.28 (m, 2H), 7.21 (br d, J=7.6 Hz, 2H), 7.15-6.96 (m, 3H), 6.75 (s, 1H), 6.70 (br d, J=8.0 Hz, 1H), 5.34 (br d, J=8.4 Hz, 1H), 4.80-4.68 (m, 1H), 4.57-4.45 (m, 1H), 4.43-4.31 (m, 1H), 4.22 (br t, J=6.8 Hz, 1H), 3.84-3.70 (m, 3H), 3.25-3.08 (m, 2H), 2.20 (s, 3H).

### Cpd_10 Synthesis

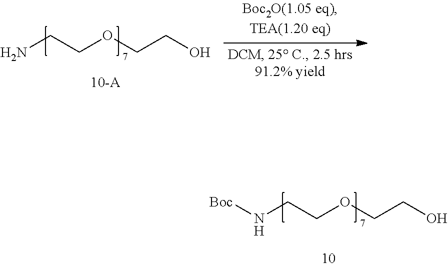

A general procedure is as follows (batch 8′-3):
1) Reaction set up: To a solution of Cpd_10-A (100 g, 270 mmol, 1.00 eq) in DCM (1000 mL) was added TEA (32.8 g, 324 mmol, 45.2 mL, 1.20 eq) and Boc₂O (62.0 g, 284 mmol, 65.2 mL, 1.05 eq). The mixture was stirred at 25° C. for 2.5 hrs.
2) Monitoring: TLC (Dichloromethane:Methanol=10:1) indicated Cpd_10-A (R_{f}=0.10) was consumed completely and two new spots (R_{f}=0.39, 0.96) formed.
3) Work up: The reaction was washed with 1M HCl (200 mL*3), H₂O (200 mL) and brine (200 mL), dried over anhydrous Na₂SO₄, filtered and concentrated to give the crude product.
4) Purification: The crude product in H₂O (300 mL) was extracted with Petroleum ether (100 mL*2). The aqueous layer was extracted with DCM (300 mL*3), the combined organic layers were dried over Na₂SO₄, filtered and concentrated under reduced pressure to give the product.
5) Result: Cpd_10 (116 g, 247 mmol, 91.2% yield, 100% purity) was obtained as light-yellow oil, checked by LCMS (Rt=0.33 min), MS cal.: 469.29, MS observed: [M+Na]⁺=492.2

HNMR (CDCl₃, Bruker_F_400 MHz) δ 5.06 (br s, 1H), 3.69-3.66 (m, 2H), 3.65-3.53 (m, 26H), 3.49 (t, J=5.2 Hz, 2H), 3.25 (t, J=5.2 Hz, 2H), 2.92 (br s, 1H), 1.39 (s, 9H).

### Cpd_11 Synthesis

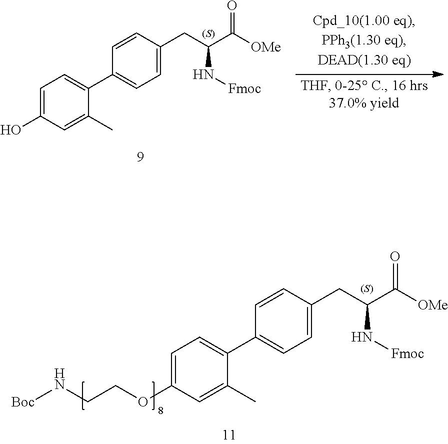

A general procedure is as follows (batch 9′-6):
1) Reaction set up: To a solution of Cpd_9 (98.0 g, 190 mmol, 1.00 eq) and Cpd_10 (89.5 g, 190 mmol, 1.00 eq) in THF (1.00 L) was added PPh₃ (65.0 g, 247 mmol, 1.30 eq) and DEAD (43.1 g, 247 mmol, 45.0 mL, 1.30 eq) dropwise at 0° C. under N₂ atmosphere. The mixture was stirred at 25° C. for 16 hrs under N₂ atmosphere.
2) Monitoring: LCMS (EC6530-707-P1A1) showed Cpd_9 was consumed completely and desired mass (Rₜ=0.55 min) detected.
3) Work up: The reaction mixture was concentrated under reduced pressure to give a residue.
4) Purification: The residue was purified by column chromatography (SiO₂, Dichloromethane: Methanol=1/0 to 8/1, Dichloromethane:Methanol=10:1, R_{f}=0.55).
5) Result: Cpd_11 (98.0 g, 70.7 mmol, 37.0% yield, 69.2% purity) was obtained as light yellow oil. HPLC(Rₜ=4.13 min), HPLC purity: 69.2% (220 nm). LCMS(Rₜ=0.55 min), MS cal.: 958.48, MS observed: [M-Boc+H]⁺=859.7

### New AA2/CPD #542 Synthesis Final Step

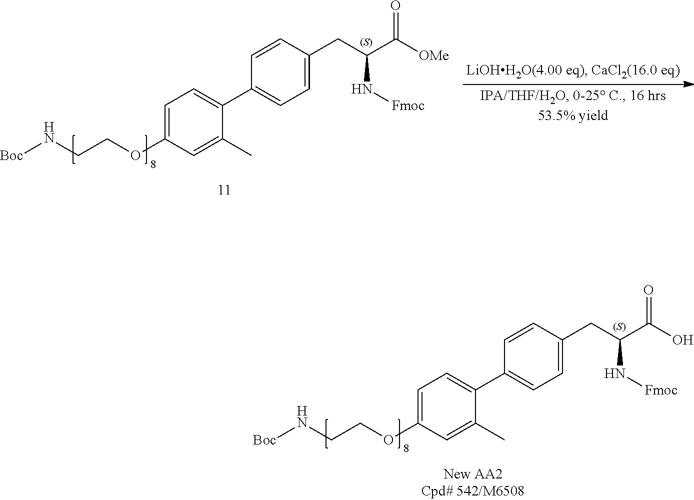

A general procedure is as follows (batch 10′-3):
1) Reaction set up: To a solution of Cpd_11 (98.0 g, 70.7 mmol, 1.00 eq) in i-PrOH (1200 mL) and THE (800 mL) was added CaCl₂ (125 g, 1.13 mol, 16.0 eq) and a solution of LiOH·H₂O (11.8 g, 282 mmol, 4.00 eq) in H₂O (400 mL) at 0° C. The mixture was stirred at 25° C. for 12 hrs.
2) Monitoring: LCMS (EC12418-4-P1A) showed Cpd_11 consumed completely, and desired mass was detected (Rₜ=0.39 min).
3) Work up: The reaction was added ice-water (1.00 L) and added 1M HCl to adjusted pH=4 5, then extracted with EtOAc (500 mL*3), the combined organic phase was washed with H₂O (500 mL) and brine (500 mL), dried over anhydrous Na₂SO₄, filtered, and concentrated to give the crude product.
4) Purification: The crude product was combined to EC22199-1 for further purification: The crude product was purified by reversed phase HPLC (0.1% HCl condition), then purified by column chromatography (SiO₂, Dichloromethane:Methanol=1/0 to 8/1, Dichloromethane:Methanol=12:1, R_{f}=0.35).
5) Result: CPD #542 (36.0 g, 37.8 mmol, 78.3% yield, 99.3% purity) was obtained as light oil. LCMS(Rₜ=2.39 min). HPLC(Rₜ=3.71 min), SFC(Rₜ=1.91 min).
   Analytical Results of Final Product New AA2/Cpd #542 (M6508) from Route 1

HNMR (CDCl₃, Bruker_F_400 MHz) δ 7.76 (br d, J=7.2 Hz, 2H), 7.57 (br s, 2H), 7.43-7.35 (m, 2H), 7.30 (t, J=7.2 Hz, 2H), 7.23-7.03 (m, 5H), 6.85-6.73 (m, 2H), 5.41 (br d, J=5.6 Hz, 1H), 5.12 (br s, 1H), 4.72 (br d, J=5.6 Hz, 1H), 4.56-4.42 (m, 1H), 4.41-4.28 (m, 1H), 4.22 (br t, J=6.0 Hz, 1H), 4.15 (br t, J=4.0 Hz, 2H), 3.87 (br t, J=4.4 Hz, 2H), 3.76-3.72 (m, 2H), 3.71-3.56 (m, 22H), 3.52 (br s, 2H), 3.37-3.09 (m, 4H), 2.21 (br s, 3H), 1.44 (s, 9H)

MS cal.: 944.47, MS observed: [M+H]=945.6; MS observed: [M-Boc+H]⁺=845.3. HPLC purity: 99.3% (220 nm). e.e. value %: 100%.

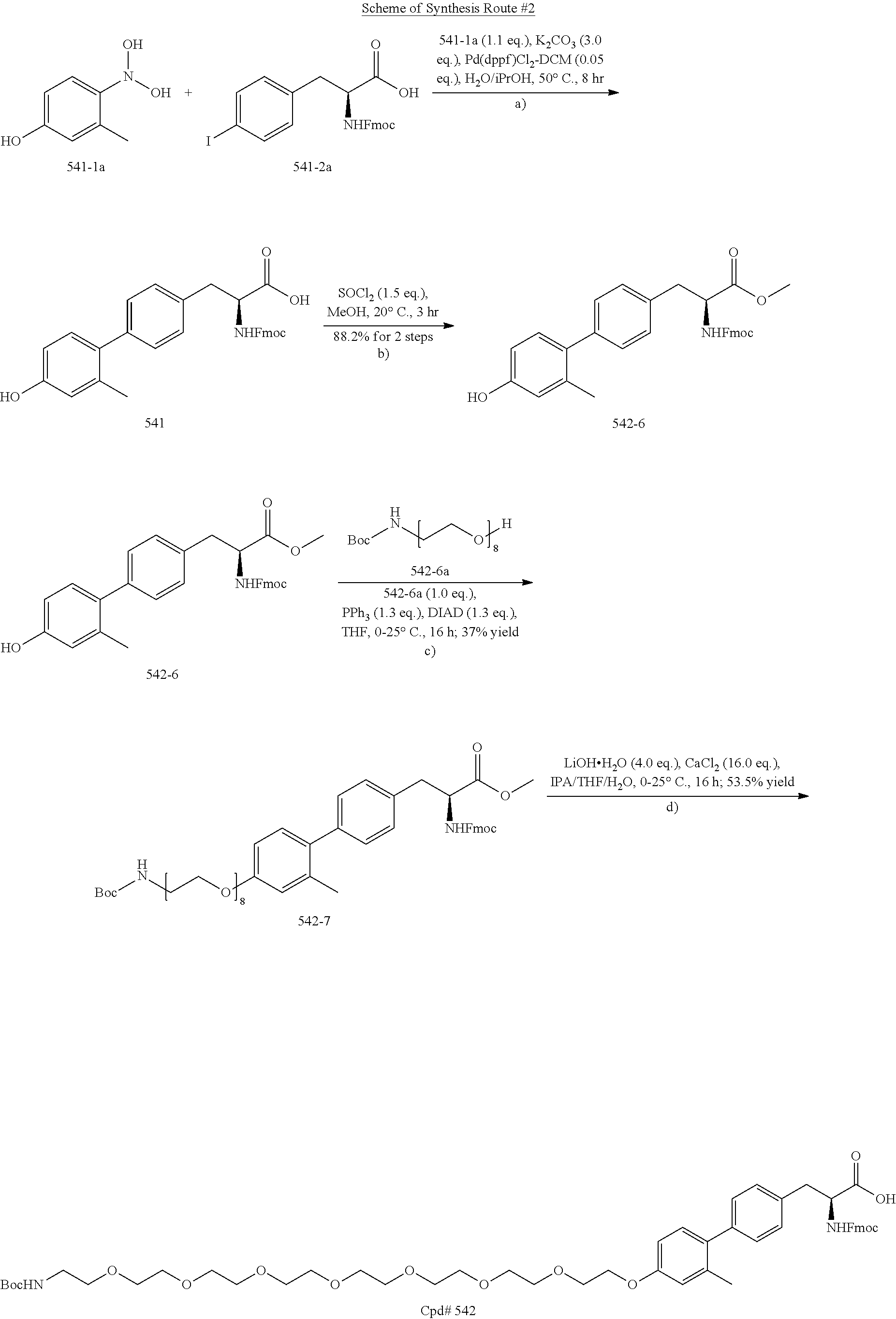

Conditions: a) CPD #541-1a (1.1 eq.), K₂CO₃ (3.0 eq.), Pd(dppf)Cl₂—CH₂Cl₂ adduct (0.05 eq.), H₂O/iPrOH, 50° C., 8 hr; b) SOCl₂ (1.5 eq.), MeOH, 20° C., 3 hr; 88.2% yield over 2 steps; c) CPD #542-6a (1.0 eq.), PPh₃ (1.3 eq.), DIAD (1.3 eq.), THF, 0-25° C., 16 h; 37% yield; d) LiOH-H₂O (4.0 eq.), CaCl₂ (16.0 eq.), iPrOH/THF/H₂O, 0-25° C., 16 h; 53.5% yield.

### Step a): Synthesis of (2S)-2-(9H-fluoren-9-ylmethoxycarbonylamino)-3-[4-(4-hydroxy-2-methyl-phenyl)-phenyl]propanoic acid (CPD #541)

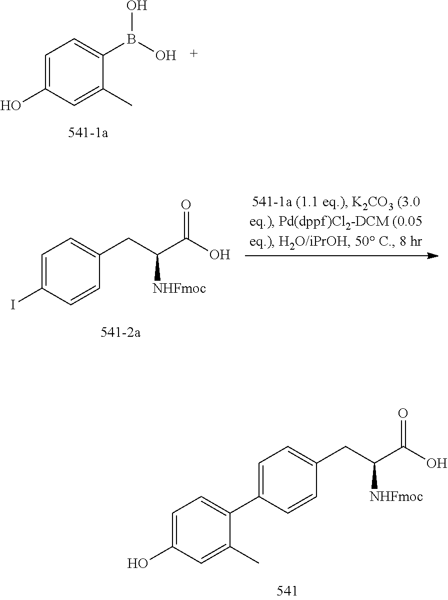

To a solution of CPD #541-1a (16.28 g, 107.14 mmol, 1.1 eq.) and CPD #541-2a (50.0 g, 97.40 mmol, 1.0 eq.) in H₂O (150 mL) and propan-2-ol (750 mL) was added Pd(dppf)Cl₂—CH₂Cl₂ adduct (3.98 g, 4.87 mmol, 0.05 eq) and K₂CO₃ (26.92 g, 194.81 mmol, 2.0 eq.). The reaction mixture was stirred at 50° C. for 8 hr. The reaction progress was monitored by LCMS. Upon completion, the mixture was extracted with EtOAc (500 mL×2). The obtained organic layers were washed with brine (500 mL×2), dried over Na₂SO₄, filtered and concentrated to give 66 g crude product CPD #541 (64 g used for next step directly).

The crude product (2 g) was purified by flash silica gel chromatography (ISCO®; 20 g SepaFlash® Silica Flash Column, Eluent of 0˜80% Ethylacetate/Petroleum ethergradient @30 mL/min) to afford CPD #541 (1.55 g, 2.88 mmol, 91.59% purity; the calculated yield for this step is 97.6%) as a brown solid.

LCMS (ESI): RT=4.611 min, m/z calcd. for C31H27O5NNa 516.18 [M+Na]⁺, found 516.0; 92.8% purity at 254 nm. Mobile Phase: 1.5 mL/4 L TFA in water (solvent A) and 0.75 mL/4 L TFA in acetonitrile (solvent B), using the elution gradient 10%-80% (solvent B) over 6 minutes and holding at 80% for 0.5 minutes at a flow rate of 0.8 mL/min; Column: Xtimate C18 2.1*30 mm, 3 um.

HPLC (ESI): RT=9.567 min; 91.59% purity at 254 nm. HPLC method A: Column: YMC-Pack ODS-A 150*4.6 mm, 5 μm; 2.75 mL/4 L TFA in water (solvent A) and 2.5 mL/4 L TFA in acetonitrile (solvent B), using the elution gradient 10%-80% (solvent B) over 10 minutes and holding at 80% for 5 minutes at a flow rate of 1.5 mL/min.

¹H NMR (400 MHz, DMSO-d₆) δ (ppm) 13.01-12.54 (m, 1H), 9.32 (br s, 1H), 7.88 (d, J=7.5 Hz, 2H), 7.80 (d, J=8.5 Hz, 1H), 7.71-7.63 (m, 2H), 7.44-7.38 (m, 2H), 7.30 (br d, J=8.4 Hz, 4H), 7.17 (d, J=8.0 Hz, 2H), 6.92 (d, J=8.3 Hz, 1H), 6.66 (d, J=2.1 Hz, 1H), 6.62 (dd, J=2.4, 8.3 Hz, 1H), 4.27-4.14 (m, 5H), 3.13 (dd, J=4.1, 13.8 Hz, 1H), 2.92 (dd, J=10.8, 13.8 Hz, 1H), 2.10 (s, 3H).

SFC: ee %: 99.36%. Conditions: Column: ChiralcelOJ-3, 50×4.6 mm I.D., 3 um; Mobile phase: A: CO₂ B: ethanol (0.05% DEA); Gradient: from 5% to 40% of B in 1.5 min and hold 40% for 1.0 min, then 5% of B for 0.5 min; Flow rate: 3 mL/min; Column temp.: 35° C.; ABPR: 1500 psi.

### Step b): Synthesis of methyl (2S)-2-(9H-fluoren-9-ylmethoxycarbonylamino)-3-[4-(4-hydroxy-2-methyl-phenyl)phenyl]propanoate (CPD #542-6)

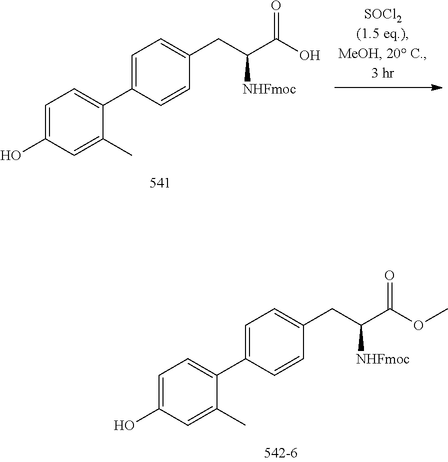

To a solution of WUX 541 (64 g crude, 94.4 mmol) in MeOH (450.0 mL) was added SOCl₂ (16.25 g, 136.61 mmol, 9.92 mL, 1.5 eq.). The mixture was stirred at 20° C. for 3 hr. The reaction progress was monitored by LCMS. Upon completion, the reaction mixture was concentrated under reduced pressure to remove MeOH. The residue was diluted with sat. aq. NaHCO₃ solution (300 mL) and extracted with DCM (250 mL×2). The combined organic layers were washed with brine (500 mL), dried (Na₂SO₄), filtered and concentrated under reduced pressure to give a residue. The residue was purified by flash silica gel chromatography (ISCO®; 330 g SepaFlash® Silica Flash Column, Eluent of 0˜100% Ethylacetate/Petroleum ethergradient @100 mL/min) to afford CPD #542-6 (44.5 g, 83.29 mmol, 88.2% yield, 97.5% purity) as a brown oil.

LCMS (ESI): RT=5.244 min, m/z calcd. for C32H2905NNa 530.19 [M+Na]⁺, found 530.1; 97.5% purity at 220 nm. Mobile Phase: 1.5 mL/4 L TFA in water (solvent A) and 0.75 mL/4 L TFA in acetonitrile (solvent B), using the elution gradient 10%-80% (solvent B) over 6 minutes and holding at 80% for 0.5 minutes at a flow rate of 0.8 mL/min; Column: Xtimate C18 2.1*30 mm, 3 um.

¹H NMR (400 MHz, DMSO-d₆) δ (ppm) 9.30 (s, 1H), 7.97-7.91 (m, 1H), 7.88 (d, J=7.5 Hz, 2H), 7.71-7.60 (m, 2H), 7.46-7.36 (m, 2H), 7.35-7.22 (m, 4H), 7.21-7.11 (m, 2H), 6.94-6.88 (m, 1H), 6.70-6.64 (m, 1H), 6.63-6.57 (m, 1H), 4.36-4.21 (m, 3H), 4.19-4.09 (m, 1H), 3.71-3.61 (m, 3H), 3.15-3.05 (m, 1H), 2.99-2.84 (m, 1H), 2.12-2.05 (m, 3H).

SFC of CPD #542-6: ee %=99.68%. Conditions: Column: Chiralpak AD-3, 50×4.6 mm I.D., 3 um; Mobile phase: A: CO₂ B: ethanol (0.05% DEA); Isocratic: 40% B; Flow rate: 4.0 mL/min; Column temp.: 35° C.; ABPR: 1500 psi.

LCMS of CPD #542-6: the purity was 97.5%.

### Step c): Synthesis of methyl (S)-2-((((9H-fluoren-9-yl)methoxy)carbonyl)amino)-3-(4′-((2,2-dimethyl-4-oxo-3,8,11,14,17,20,23,26-octaoxa-5-azaoctacosan-28-yl)oxy)-2′-methyl-[1,1′-biphenyl]-4-yl)propanoate (CPD #542-7)

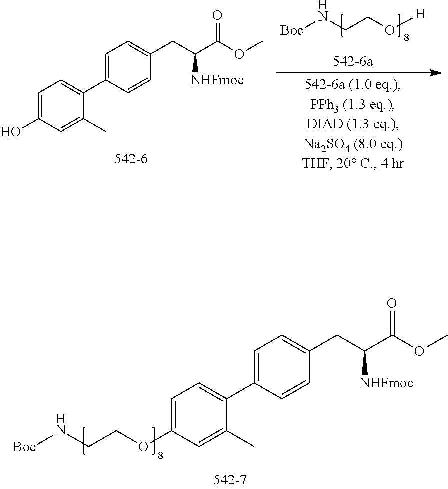

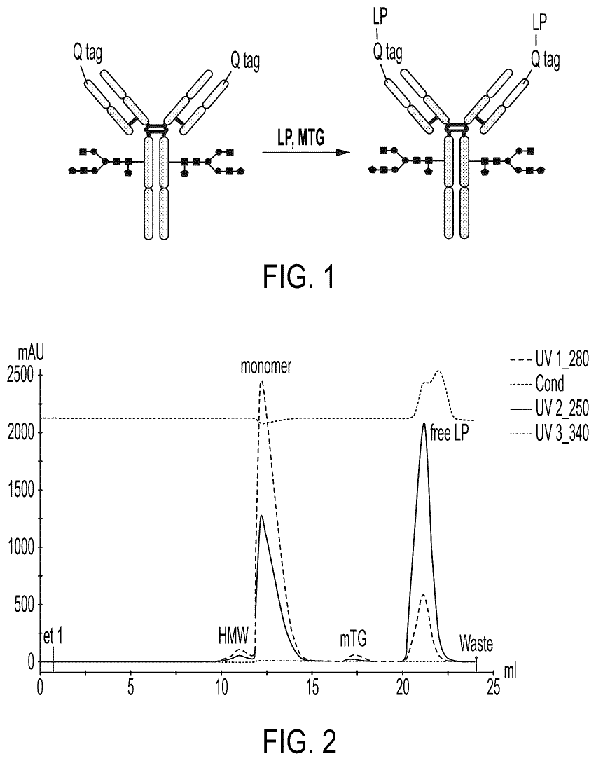

To a solution of CPD #542-6a (92.51 mg, 197.02 mol, 1.0 eq.) in THF (1.0 mL) was added DIAD (51.79 mg, 256.12 mol, 49.65 μL, 1.3 eq.), PPh₃ (67.18 mg, 256.12 mol, 1.3 eq.), CPD #542-6 (100.0 mg, 197.02 mol, 1.0 eq.) and Na₂SO₄ (223.88 mg, 1.58 mmol, 159.91 L, 8.0 eq.). The mixture was stirred at 20° C. for 4 hr. LCMS showed the conversion % of CPD #542-6 to CPD #542-7 was 33% (FIG. 1).

LCMS: (ESI): RT=6.114 min, m/z calcd. for C53H70014N2Na 981.47 [M+Na]f, found 981.2. Mobile Phase: 1.5 mL/4 L TFA in water (solvent A) and 0.75 mL/4 L TFA in acetonitrile (solvent B), using the elution gradient 10%-80% (solvent B) over 6 minutes and holding at 80% for 0.5 minutes at a flow rate of 0.8 ml/min; Column: Xtimate C18 2.1*30 mm, 3 um.

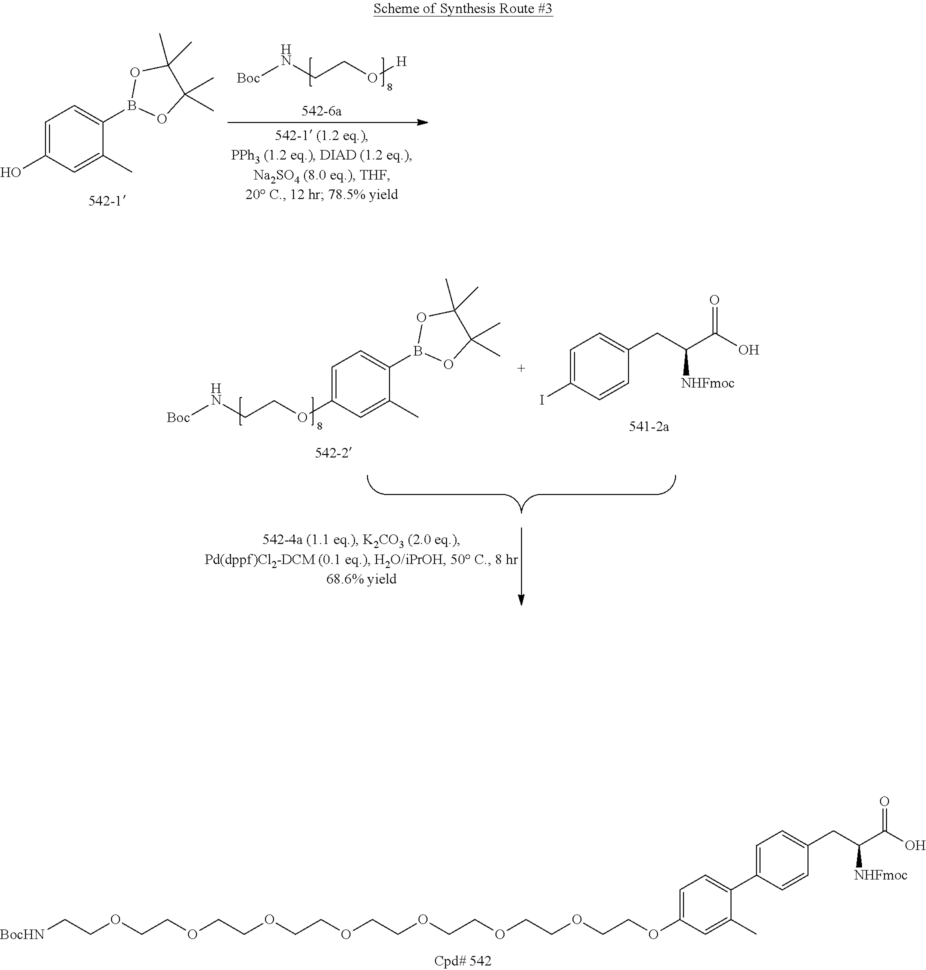

Conditions: a) CPD #542-1′ (1.2 eq.), PPh₃ (1.2 eq.), DIAD (1.2 eq.), Na₂SO₄ (8.0 eq.), THF, 20° C., 12 hr; 78.5% yield; b) CPD #541-2a (1.1 eq.), K₂CO₃ (2.0 eq.), Pd(dppf)Cl₂—CH₂Cl₂ adduct (0.1 eq.), H2O/iPrOH, 50° C., 8 hr; 68.6% yield.

### Step a): Synthesis of tert-butyl N-[2-[2-[2-[2-[2-[2-[2-[2-[3-methyl-4-(4,4,5,5-tetramethyl-1,3,2-dioxaborolan-2-yl)phenoxy]ethoxy]ethoxy]ethoxy]ethoxy]ethoxy]ethoxy]ethoxy]ethyl]carbamate (CPD #542-2′)

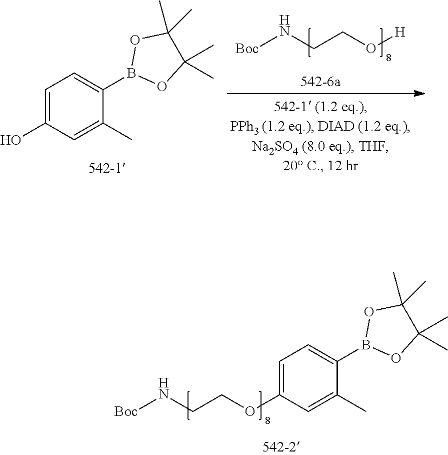

To a mixture of CPD #542-1′ (1.2 eq.), PPh₃ (1.2 eq.), CPD #542-6a (1.0 eq.) and Na₂SO₄ (8.0 eq.) in THF (volume for the corresponding c [CPD #542-1′]) was added DIAD (1.2 eq.). The reaction mixture was stirred at 20° C. for 12 hr. The results are following:

| batch | CPD# 542-1′ | c [CPD# 542-1′] (mM) | Conv. % (to CPD# 542-2′) |
|---|---|---|---|
| 1 | 15 mg | 63 | 0 (No reaction) |
| 2 | 281 mg | 394.4 | 52.9 |
| 3 | 598 mg | 574.8 | 90.4 |

Conditions: CPD# 542-1′ (1.2 eq.), CPD# 542-6a (1.0 eq.), PPh₃ (1.2 eq.), Na₂SO₄ (8.0 eq.), DIAD (1.2 eq.), THF; then DIAD (1.2 eq.) was added dropwise; 20° C., 12 hr.

To a mixture of CPD #542-1′ (3.59 g, 15.33 mmol, 1.2 eq.), PPh₃ (4.02 g, 15.33 mmol, 1.2 eq.), CPD #542-6a (6.0 g, 12.78 mmol, 1.0 eq.) and Na₂SO₄ (14.52 g, 102.22 mmol, 10.37 mL, 8.0 eq.) in THF (10.0 mL) was added DIAD (3.10 g, 15.33 mmol, 2.97 mL, 1.2 eq.). The reaction mixture was stirred at 20° C. for 12 hr. The reaction progress was monitored by LCMS (FIG. 2). The conversion % of CPD #542-6a to CPD #542-2′ was 88.9%. Upon completion, the reaction mixture was filtered, and the filtrate was concentrated under reduced pressure give a residue. The residue was purified by flash silica gel chromatography (ISCO®; 120 g SepaFlash® Silica Flash Column, Eluent of 0˜100% EtOAc/PE gradient @80 mL/min for 35 min with total volume 2.8 L) to afford CPD #542-2′ (8.6 g, 10.03 mmol, 78.5% yield, 80% purity) as a yellow oil.

LCMS (ESI): RT=1.065 min, m/z, calcd. for C34H60BNO12Na 708.42 [M+Na]⁺, found 708.5. LC-MS Conditions: Mobile Phase:1.5 mL/4LTFA in water (solvent A) and 0.75 mL/4LTFA in acetonitrile (solvent B), using the elution gradient 5%-95% (solvent B) over 0.7 minutes and holding at 95% for 0.4 minutes at a flow rate of 1.5 mL/min.

¹H NMR (400 MHz, CDCl₃) δ (ppm) 7.70-7.69 (m, 1H), 6.76-6.69 (m, 2H), 4.18-4.15 (m, 2H), 3.88-3.84 (m, 2H), 3.75-3.72 (m, 2H), 3.70-3.61 (m, 23H), 3.55 (t, J=5.1 Hz, 2H), 3.37-3.26 (m, 2H), 2.52 (s, 3H), 1.46 (s, 9H), 1.34 (s, 12H).

### Step b): Synthesis of (2S)-3-[4-[4-[2-[2-[2-[2-[2-[2-[2-[2-(tert-butoxycarbonylamino)ethoxy]ethoxy]ethoxy]ethoxy]ethoxy]ethoxy]ethoxy]ethoxy]-2-methyl-phenyl]phenyl]-2-(9H-fluoren-9-ylmethoxy-carbonylamino)propanoic acid (CPD #542)

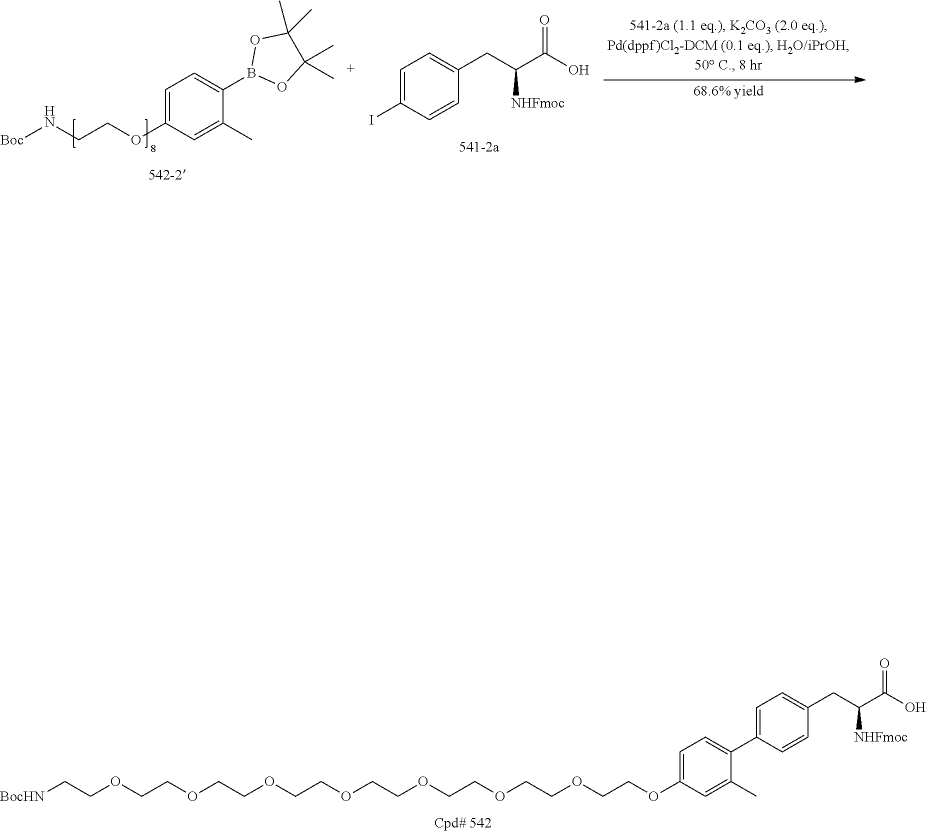

To a solution of CPD #542-2′ (4.0 g, 80% purity, 4.67 mmol, 1.0 eq.) and CPD #541-2a (2.64 g, 5.13 mmol, 1.1 eq.) in H₂O (10.0 mL) and i-PrOH (40.0 mL) were added Pd(dppf)Cl₂—CH₂Cl₂ adduct (381.14 mg, 466.71 mol, 0.1 eq.) and K₂CO₃ (1.29 g, 9.33 mmol, 2.0 eq.). The reaction mixture was stirred at 50° C. for 8 hr. The reaction progress was monitored by LCMS. Upon completion, the reaction mixture was quenched by citric acid to pH 5 and extracted with EtOAc (100 mL×2). The combined organic layers were washed with brine (80 mL×2), dried over Na₂SO₄, filtered, and concentrated under reduced pressure to give a residue. The residue was purified by reverse column chromatography (C18 330 g, Eluent of 0˜₆₀% CH₃CN/water (+0.225% HOAc) gradient @60 mL/min for 15 min with total volume 0.9 L) to afford CPD #542 (3.1 g, 3.20 mmol, 68.6% yield, 97.61% purity) as a yellow oil.

LCMS (ESI): RT=4.197 min, m/z, calcd. for C52H68N₂O14Na 967.47 [M+Na]⁺, found 967.3; 97.61% purity at 220 nm. LC-MS Conditions: Mobile Phase: 1.5 mL/4 L TFA in water (solvent A) and 0.75 mL/4 L TFA in acetonitrile (solvent B), using the elution gradient 30%-90% (solvent B) over 6 minutes and holding at 90% for 0.5 minutes at a flow rate of 0.8 ml/min; Column: Nano Chrom 120 C18 3.0*30 mm, 3 um.

HPLC: RT=9.006 min; 98.4% purity at 220 nm. HPLC conditions: Mobile Phase: 2.75 mL/4 L TFA in water (solvent A) and 2.5 mL/4 L TFA in acetonitrile (solvent B), using the elution gradient 30%-90% (solvent B) over 10 minutes and holding at 90% for 5 minutes at a flow rate of 1.5 mL/min; Column: Ultimate LP-C18 150*4.6 mm 5 um.

¹H NMR (400 MHz, CDCl₃) δ (ppm) 7.78 (d, J=7.5 Hz, 2H), 7.63-7.56 (m, 2H), 7.44-7.37 (m, 2H), 7.35-7.29 (m, 2H), 7.25-7.08 (m, 5H), 6.87-6.74 (m, 2H), 5.43-5.32 (m, 1H), 5.19-5.08 (m, 1H), 4.80-4.69 (m, 1H), 4.51 (br dd, J=7.3, 10.0 Hz, 1H), 4.42-4.33 (m, 1H), 4.24 (br t, J=6.9 Hz, 1H), 4.17 (br d, J=4.0 Hz, 2H), 3.92-3.84 (m, 2H), 3.80-3.73 (m, 2H), 3.72-3.50 (m, 25H), 3.35-3.16 (m, 4H), 2.26-2.18 (m, 3H), 1.49-1.43 (m, 9H).

SFC: ee %: 99.56%. Conditions: Column: Chiralcel OJ-3, 50×4.6 mm I.D., 3 um; Mobile phase: A: CO₂ B: ethanol (0.05% DEA); Gradient: from 5% to 40% of B in 1.5 min and hold 40% for 1.0 min, then 5% of B for 0.5 min; Flow rate: 3 mL/min Column temp.: 35° C.; ABPR: 1500 psi.

### Example 13. Experimental Section for the Synthesis of M6324

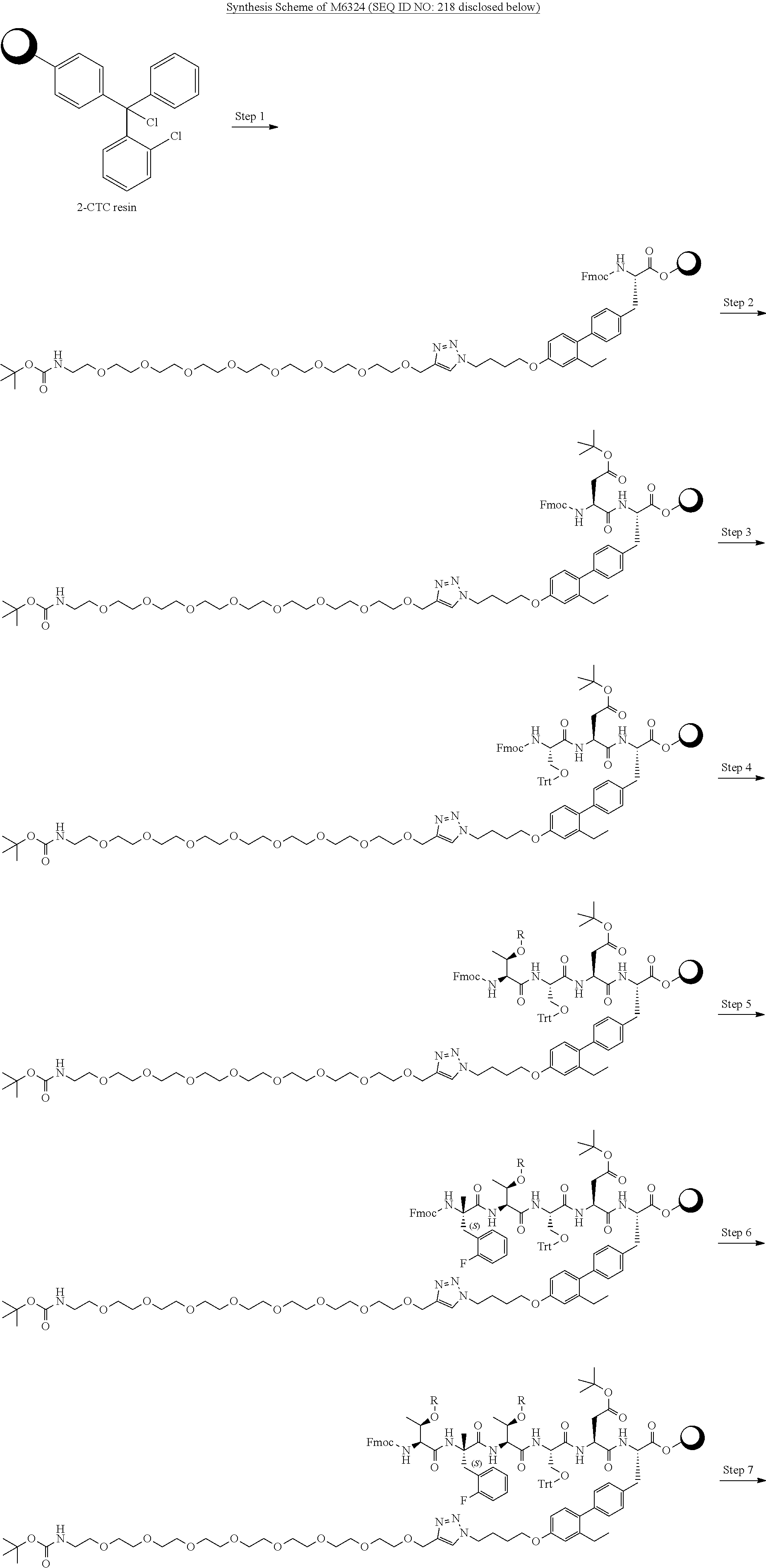

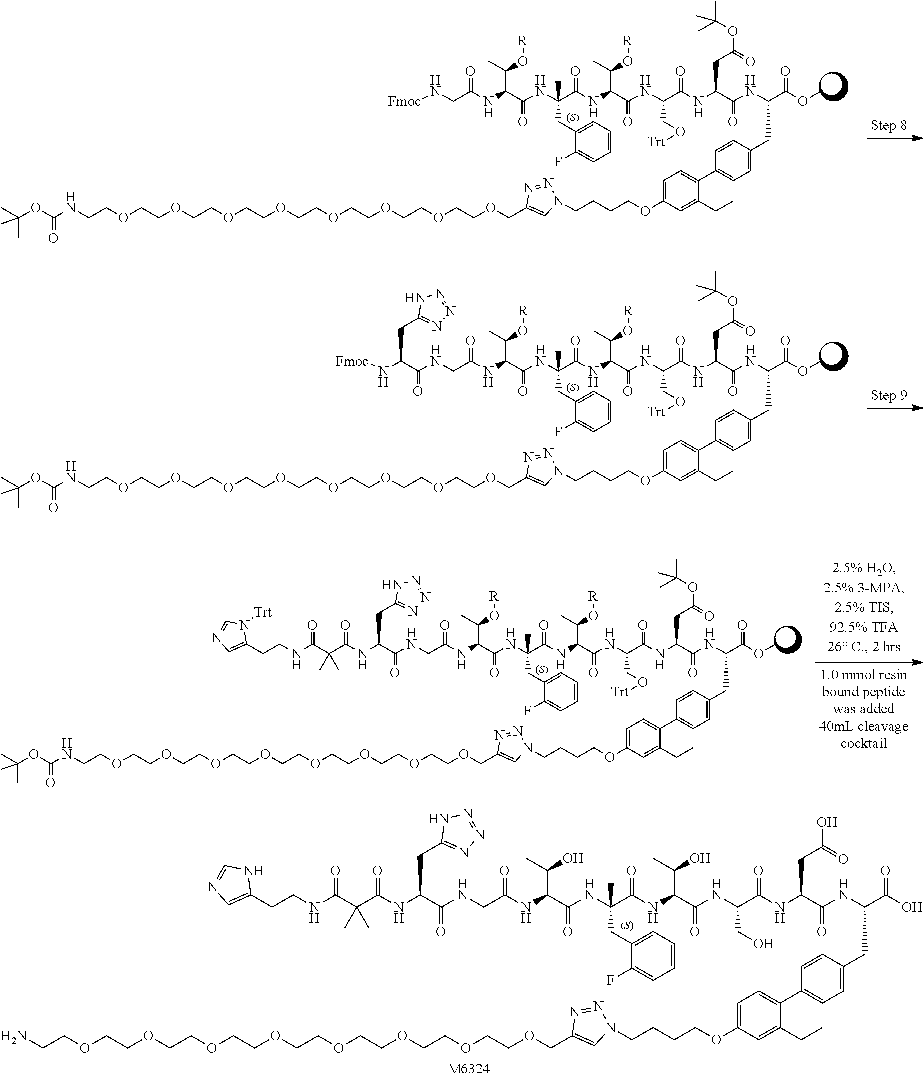

### 13.1 Procedures of M6324 Synthesis and Purification

M6324 was synthesized by standard Fmoc chemistry under solid phase synthesis conditions, using Fmoc-AA2 (Boc-Linker)-OH, AA9 and AA10, and other natural amino acids. The details for lot 1 (1 mmol scale) and for lot 2 (20.0 mmol, 40.0 g 2-CTC resin, Sub: 0.5 mmol/g) using same reaction conditions are described in detail below.
1) Resin preparation: To a solution the 2-CTC resin (40.0 g, 20.0 mmol, 1.00 eq, Sub 0.5 mmol/g) in DCM (1.0 L) was added Fmoc-AA2 (Boc-Linker)-OH (1.0 eq) and DIEA (3.0 eq). The mixture was agitated with N₂ for 3 hr.
2) Deprotection: 20% piperidine in DMF (1.0 L) was added and agitated the resin with N₂ for another 20 mins. The resin was washed with DMF (1.0 L) 5 times and filtered to get the resin. The deprotection was monitored by ninhydrin color reaction.
3) Coupling: A solution of Fmoc-Asp(OtBu)-OH (3.0 eq), DIC (3.0 eq) and HOAt (3.0 eq) in DMF (500 mL) was added to the resin and agitated with N₂ for 100 min. The resin was washed with DMF (1.0 L) 5 times and filtered to get the resin. The coupling reaction was monitored by ninhydrin color reaction.
4) Deprotection: 20% piperidine in DMF (1.0 L) was added and agitated the resin with N₂ for another 20 mins. The resin was washed with DMF (1.0 L) 5 times and filtered to get the resin. The deprotection was monitored by ninhydrin color reaction.
5) Coupling: A solution of Fmoc-Ser(Trt)-OH (3.0 eq), DIC (3.0 eq) and HOAt (3.0 eq) in DMF (500 mL) was added to the resin and agitated with N₂ for 80 mins. The resin was washed with DMF (1.0 L) 5 times and filtered to get the resin. The coupling reaction was monitored by ninhydrin color reaction.
6) Deprotection: 20% piperidine in DMF (1.0 L) was added and agitated the resin with N₂ for another 20 mins. The resin was washed with DMF (1.0 L) 5 times and filtered to get the resin. The deprotection was monitored by ninhydrin color reaction.
7) Coupling: A solution of Fmoc-Thr-OH or Fmoc-Thr(tBu)-OH (3.0 eq), DIC (3.0 eq) and HOAt (3.0 eq) in DMF (500 mL) was added to the resin and agitated with N₂ for 60 mins. The resin was washed with DMF (1.0 L) 5 times and filtered to get the resin. The coupling reaction was monitored by ninhydrin color reaction.
8) Repeat above step 2 to 3 for the coupling of following amino acids Fmoc-a-Me-Phe(2-F)-OH (3.0 eq), Fmoc-Thr-OH or Fmoc-Thr(tBu)-OH (3.0 eq), Fmoc-Gly-OH (3.0 eq), AA9 and AA10, the coupling regents uses DIC/HOAt and is equivalent to the amino acid.
9) Added 30% NH₂NH₂ in DMF (1.0 L) to the resin and agitated with N₂ for 30 mins. The resin was washed with DMF (1.0 L) 5 times and filtered to get the resin.
10) The resin was washed with MeOH (1.0 L) for 3 times and dried under vacuum and to get the peptide resin 80 g.
11) 800 mL of cleavage buffer (2.5% H₂O, 2.5% 3-MPA, 2.5% TIS, and 92.5% TFA) into flask and stirred with magnetic stirrer.
12) The remaining 80 g of resin bound M6324 was added to the flask slowly and stirred at room temperature (26° C.) for 2 hours.
13) Filter and concentrated the TFA cocktail by 70% and precipitated with 20V isopropyl ether.
14) The cleavage buffer containing the peptide (M6324) was slowly added to cold isopropyl ether (2 L) with stirred. The peptide was precipitated. The mixture was centrifuged (2 min at 3000 rpm) and the precipitate was collected and washed two times with isopropyl ether (2 L).
15) The crude peptide M6324 was dried under vacuum for 2 hours to generate 28 g as white solid.

### 13.2 Purification of M6324

The reaction mixture was purified by prep-HPLC: mobile phase A: 0.075% TFA in H2O; mobile phase B: ACN; 15% B-45% B-50 min gradient elution.
1) For lot 1: All fractions around 94.86% purity were collected and then lyophilized to obtain pure M6324 TFA salt (311.2 mg, 94.86% purity) as a white solid in 17% yield.
2) For lot 2: All fractions around 97.25% purity were collected and then lyophilized to obtain pure M6324 TFA salt (11930 mg, 97.25% purity) as a white solid in 27% yield.
3) The details of the purification conditions are listed below.

| Dissolution condition | 2 g Crude M6324 lot1 or 28 g | |
|---|---|---|
| | Crude M6324 lot2 was dissolved | |
| | in 25% ACN—H2O. | |
| Sample loading amounts / | 9.4 g per injection, | |
| Injections | 3 injections in total. | |
| Instrument | Qing bohua DAC100 | |
| Mobile Phase | A: ACN | |
| | B: H₂O (0.075% TFA in H₂O) | |
| Gradient | Time | A % |
| | 0 | 5 |
| | 3 | 20 |
| | 53 | 40 |
| | 53.1 | 95 |
| | 60 | 95 |
| | 60.1 | 5 |
| | 68 | 5 |
| Column | luna c18 10 um 100A, | |
| | length: 25 cm, | |
| | diameter: 10 cm. | |
| Flow Rate | 200 | mL/Min |
| Wavelength | 214/254 | nm |
| Oven Tem. | 26° | C. |

### 13.3Analytical Results for M6324

Lot #1: The purity is 94.86% with M6324 in 220 nm. LCMS shows a main peak at retention time of 1.110 min which corresponds to the desired product with the observed mass of [M+H]⁺=1775.8.

Lot #2: The purity is 97.25% at 220 nm. LCMS shows a main peak at retention time of 1.025 min which corresponds to the desired product with the observed mass of [M+H]+=1775.8.

### HNMR of M6324:

¹H NMR (400 MHz, METHANOL-d₄) δ ppm 8.44 (1H, d, J=1.2 Hz), 8.07 (1H, s), 7.71 (1H, s), 7.29 (1H, d, J=6.0 Hz), 7.00 (1H, s), 6.91 (2H, d, J=8.0 Hz), 6.82-6.89 (1H, m), 6.75-6.70 (4H, m), 6.62-6.68 (1H, m), 6.57 (1H, t, J=9.2 Hz), 6.50 (1H, d, J=2.6 Hz), 6.43 (1H, dd, J=8.4, 2.6 Hz), 4.52 (4H, br dd, J=8.0, 5.1 Hz), 4.42 (1H, br dd, J=8.4, 5.1 Hz), 4.30-4.35 (3H, m), 4.19 (2H, t, J=7.2 Hz), 4.02-4.11 (3H, m), 3.93 (1H, dd, J=6.4, 4.4 Hz), 3.8 3 (1H, dd, J=6.0, 4.3 Hz), 3.62-3.7 5(5H, m), 3.5 5(1H, dd, J=11.4, 4.1 Hz), 3.3 9-3.44 (2H, m), 3.33-3.36 (6H, m), 3.31 (8H, s), 3.28-3.30 (6H, m), 3.08-3.28 (4H, m), 2.95-3.00 (3H, m), 2.89 (1H, br dd, J=13.8, 4.8 Hz), 2.83 (2H, br t, J=5.0 Hz), 2.67 (1H, dd, J=13.6, 9.4 Hz), 2.60 (2H, t, J=6.6 Hz), 2.28-2.40 (2H, m), 2.19 (2H, br dd, J=7.5, 2.8 Hz), 1.76-1.84 (2 H, m), 1.48 (2H, br dd, J=8.6, 6.2 Hz), 1.11 (3H, s), 0.98-1.06 (6H, m), 0.91 (6H, dd, J=6.2, 3.4 Hz), 0.71 (3H, t, J=7.4 Hz).

SFC of M6324 lot 2: Method details: Column: Chiralcel OJ-RH 150×4.6 mm I.D., 5 um

Mobile phase: A: water with 0.0375% TFA B: acetonitrile with 0.01875% TFA B in A from 10% to 80%. Flow rate: 1.0 mL/min; Wavelength: 220n.

### Example 14. Experimental Section for the Synthesis of M6447 (SEQ ID NO: 336 Disclosed Below)

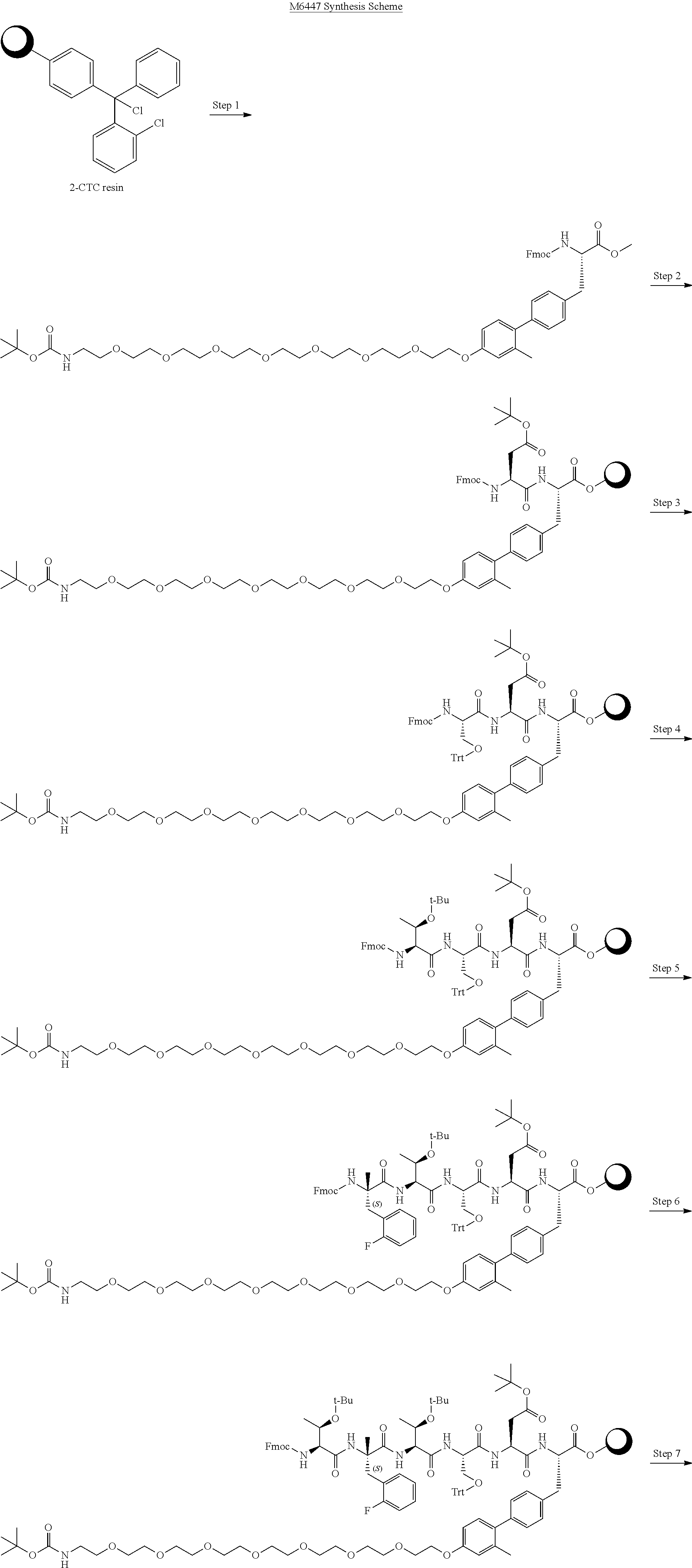

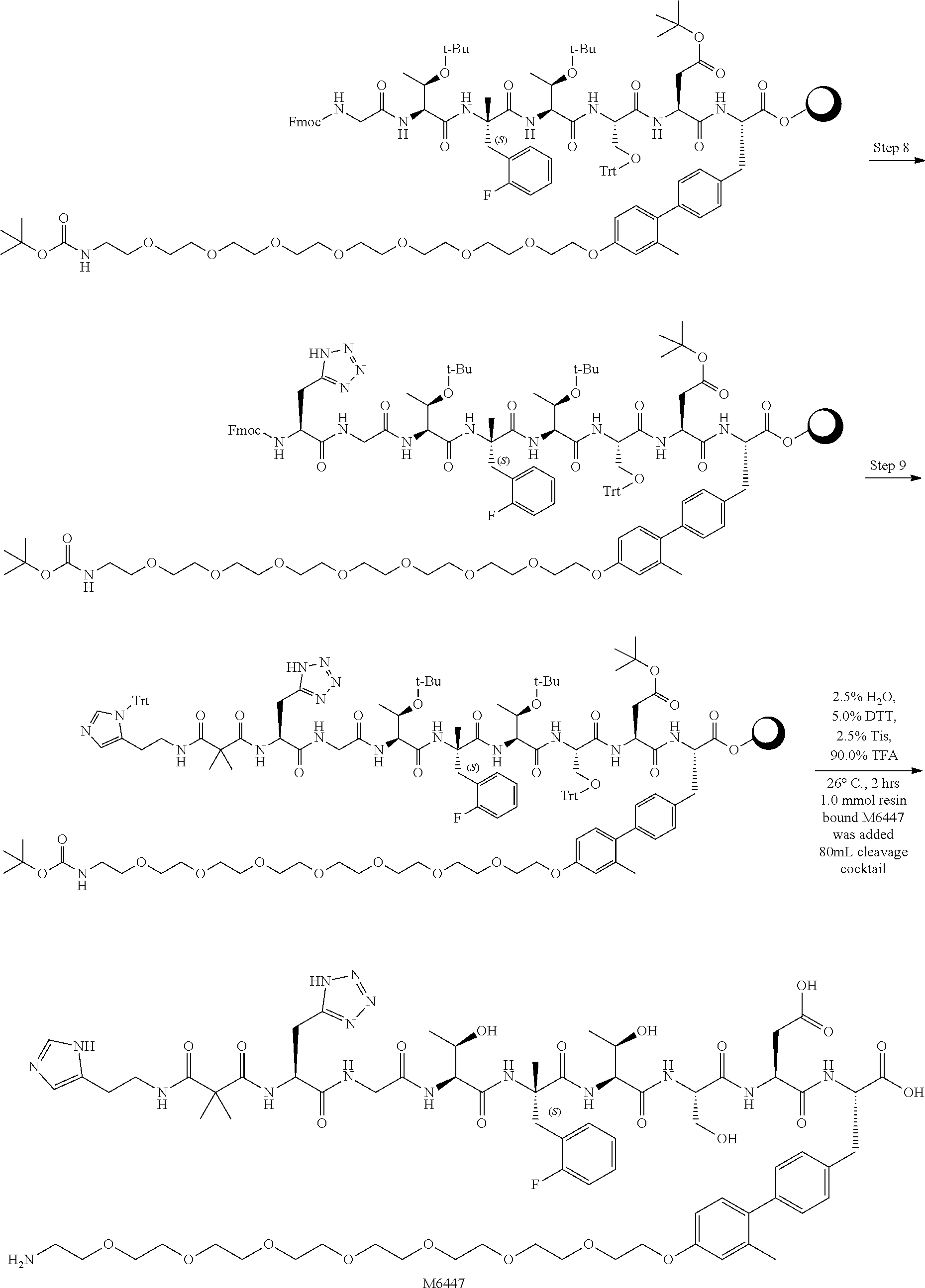

### 14.1 Procedures of M6447 Synthesis and Purification

M6447 was synthesized by standard Fmoc chemistry under solid phase synthesis conditions, using new AA2 (CPD #542), AA9 and AA10, and other natural amino acids. The details for lot 1 (1 mmol scale) were recorded on Notebook Page EW50192-3) and for lot 2 (20.0 mmol, 40.0 g 2-CTC resin, Sub: 0.5 mmol/g) using same reaction conditions were listed following. The M6447 was synthesized by solid phase synthesis using CPD #542, AA9 and AA10.

A general composition procedure is as follows:
1) Resin preparation: To a solution the 2-CTC resin (40.0 g, 20.0 mmol, 1.00 eq, Sub 0.5 mmol/g) in DCM (1 L) was added New AA2 (1.0 eq) and DIEA (3.0 eq). The mixture was agitated with N₂ for 180 min.
2) Deprotection: 20% piperidine in DMF (1 L) was added and agitated the resin with N₂ for another 20 mins. The resin was washed with DMF (1 L) 5 times and filtered to get the resin. The deprotection was monitored by ninhydrin color reaction.
3) Coupling: A solution of Fmoc-Asp(OtBu)-OH (3.0 eq), DIC (3.0 eq) and Oxyma (3.0 eq) in DMF (500 mL) was added to the resin and agitated with N₂ for 16 hr. The resin was washed with DMF (1 L) 5 times and filtered to get the resin. The coupling reaction was monitored by ninhydrin color reaction.
4) Deprotection: 20% piperidine in DMF (1 L) was added and agitated the resin with N₂ for another 20 mins. The resin was washed with DMF (1 L) 5 times and filtered to get the resin. The deprotection was monitored by ninhydrin color reaction.
5) Coupling: A solution of Fmoc-Ser(Trt)-OH (3.0 eq), DIC (3.0 eq) and Oxyma (3.0 eq) in DMF (500 mL) was added to the resin and agitated with N₂ for 90 min. The resin was washed with DMF (1 L) 5 times and filtered to get the resin. The coupling reaction was monitored by ninhydrin color reaction.
6) Deprotection: 20% piperidine in DMF (1 L) was added and agitated the resin with N₂ for another 20 mins. The resin was washed with DMF (1 L) 5 times and filtered to get the resin. The deprotection was monitored by ninhydrin color reaction.
7) Coupling: A solution of Fmoc-Thr(tBu)-OH (3.0 eq), DIC (3.0 eq) and Oxyma (3.0 eq) in DMF (500 mL) was added to the resin and agitated with N₂ for 80 mins. The resin was washed with DMF (1 L) 5 times and filtered to get the resin. The coupling reaction was monitored by ninhydrin color reaction.
8) Repeat above step 2 to 3 for the coupling of following amino acids Fmoc-a-Me-Phe(2-F)-OH (3.0 eq), Fmoc-Thr(tBu)-OH (3.0 eq), Fmoc-Gly-OH (3.0 eq), AA9 and AA10, the coupling regents uses DIC/Oxyma and is equivalent to the amino acid.
9) The resin was washed with MeOH (1 L) for 3 times and dried under vacuum and to get the peptide resin 80 g.
10) 1.6 L of cleavage buffer (2.5% H₂O, 5.0% DTT, 2.5% TIS, and 92.5% TFA) into flask and stirred with magnetic stirrer.
11) The remaining 80 g of resin bound M6447 was added to the flask slowly and stirred at room temperature (26° C.) for 2 hours.
12) Filter and concentrated the TFA cocktail by 50% and precipitated with 1OV isopropyl ether.
13) The cleavage buffer containing the peptide (M6447) was slowly added to cold isopropyl ether (8 L) with stirred. The peptide was precipitated with isopropyl ether (1 L) and centrifuged (2 min at 3000 rpm) and the precipitate was collected and washed two times with isopropyl ether (2 L).
14) The crude peptide M6447 was dried under vacuum for 2 hours to generate 35 g as white solid.

### 14.2 Purification of M6447

The reaction mixture was purified by prep-HPLC: mobile phase A: 0.075% TFA in H₂O; mobile phase B: ACN; 15% B-35% B-50 min gradient elution.

For lot1: all fractions around 96.37% purity were collected and then lyophilized to obtain pure M6447 TFA salt (250.1 mg, 96.37% purity) as a white solid in 15% yield. The detail of purification conditions as below.

For lot2: All fractions around 98.25% purity were collected and then lyophilized to obtain pure M6447 (13.56 g, 98.25% purity, TFA salt) as a white solid in 36.25% yield. The detail of purification conditions as below.

| Dissolution condition | 35 g Crude M6447 dissolved | |
|---|---|---|
| | in 360 mL H2O + 40 mL ACN. | |
| Sample loading amounts/ | 8.75 g per injection, 4 injections | |
| Injections | in total. | |
| Instrument | Qing bohua DAC100 | |
| Mobile Phase | A: ACN | |
| | B: H₂O (0.075% TFA in H₂O) | |
| Gradient | Time | A % |
| | 0 | 5 |
| | 3 | 20 |
| | 53 | 40 |
| | 53.1 | 95 |
| | 60 | 95 |
| | 60.1 | 5 |
| | 68 | 5 |
| Column | luna c18 10 um 100A, | |
| | length:25 cm, diameter: 10 cm. | |
| Flow Rate | 200 | mL/Min |
| Wavelength | 214/254 | nm |
| Oven Tem. | 26° | C. |

### 14.3 Analytical Results of M6447

Lot #1: The purity is 96.37% at 220 nm. LCMS shows a main peak at retention time of 1.052 min, which corresponds to the desired product with the observed mass of [M+H]+=1608.77, [M+2H]²⁺=805.39.

Lot #2: The purity is 98.25% at 220 nm. LCMS shows a main peak at retention time of 0.978 min which corresponds to the desired product M6447 with the observed mass of [M+H]⁺=1609.74, [M+2H]²⁺=805.36.

SFC of M6447: Method details: Column: Chiralcel OJ-RH 150×4.6 mm I.D., Sum

Mobile phase: A: water with 0.0375%; TFA B: acetonitrile with 0.01875% TFA B in A from 10% to 80%

Flow rate: 1.0 mL/min; Wavelength: 220 nm.

### Example 15—Preparation of Antibody-Tethered Ligands (ATLs) REGN18121-M6324, REGN18121-M6009, and the Corresponding Isotype Control ATLs

### 15.1 Generation of Antibody-Tethered Ligands

Anti-GLP1R human IgG antibody or isotype control antibody containing a Q-tag was mixed with 10-20 molar equivalent of linker payload (FIG. 1). Tris-HCl was added to adjust pH to 7-8. The resulting solution was mixed with microbial transglutaminase (0.5-5 units MTG per mg of antibody) resulting in a final concentration of the antibody at 5-20 mg/mL. The reaction mixture was incubated at 25-37° C. for 2-8 hours with gently shaking while reaction was monitored by ESI-MS. Upon the completion, the excess amount of linker-payload, protein aggregates, and MTG were removed by size exclusion chromatography (SEC) or Protein A affinity chromatography (ProA). The purified conjugate was concentrated, buffer exchanged into the formulation buffer and sterile filtered. The protein concentration was measured by a UV-vis spectrophotometer. Overall protein recovery was 50-85%. ATL monomer purity (>90%) was determined by analytical SEC. ATLs were further characterized by liquid chromatography electrospray ionization mass spectrometry (LC-ESI-MS) to calculate the drug-to-antibody ratio (DAR>1.8) and hydrophobic interaction chromatography (HIC) to compare conjugates hydrophobicity.

### 15.2 Site-Specific Conjugation of Linker Payload M6324 to the Anti-GLP1R Antibody REGN18121

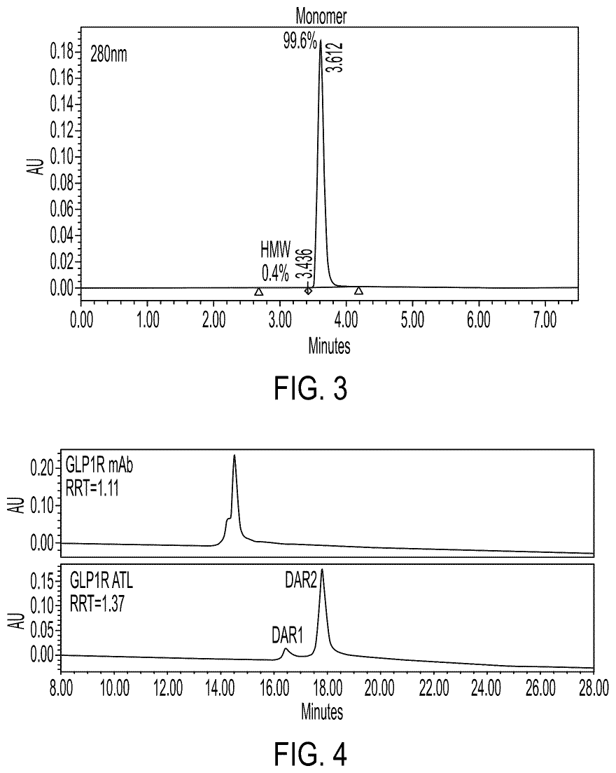

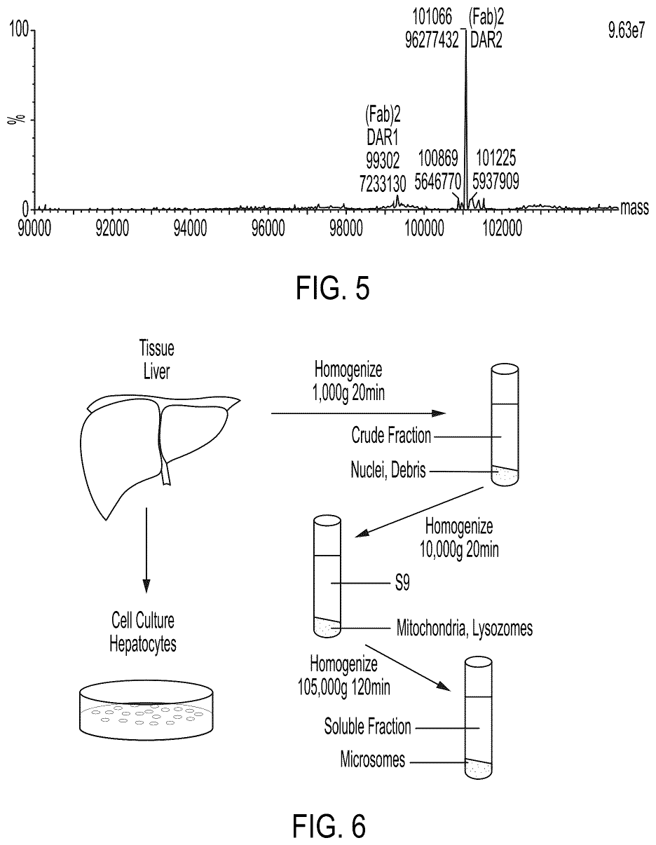

Anti-GLP1R antibody REGN18121 containing a light chain N-term Q-tag (10 mg) was mixed with 15 molar equivalents of linker-payload (M6423). 1M Tris-HCl pH 8 was added to bring the Tris concentration to 75 mM (pH ˜7.8). The resulting solution was mixed with microbial transglutaminase (1 unit MTG per mg of antibody) resulting in a final concentration of the antibody of 11 mg/mL. The reaction mixture was incubated at 37° C. for 3 hours with gentle shaking. The reaction was monitored by ESI-MS. Upon completion, excess amount of linker-payload, protein aggregates, and MTG were removed by size exclusion chromatography (SEC) (AKTA pure, Superdex 200 pg) (FIG. 2). The purified conjugate was dialyzed into formulation buffer (20 mM sodium acetate pH 5, 8% sucrose, 0.1% PS80) and sterile filtered. The protein concentration was measured by a UV-vis spectrophotometer. Overall protein recovery was 75% (Table 16). ATL monomer purity was determined by analytical SEC (FIG. 3). ATLs were further characterized by liquid chromatography electrospray ionization mass spectrometry (LC-ESI-MS) to calculate the drug-to-antibody ratio (FIG. 4) and by hydrophobic interaction chromatography (HIC) (FIG. 5).

TABLE 16
List of Antibody-Tethered Ligands (ATL)

| | | Linker- | ATL Products | |
|---|---|---|---|---|
| Target Ab | Ab # | Payload | Yield | LCMS DAR |
| GLP1R antibody | REGN18121 | M6009 | 81% | 1.9 |
| GLP1R antibody | REGN18121 | M6324 | 75% | 1.9 |
| Isotype control | REGN7438 | M6009 | 75% | 1.9 |
| Isotype control | REGN7438 | M6324 | 71% | 2.0 |

### 15.3 Characterization of Antibody-Tethered Ligand

Analytical size exclusion chromatography (SEC) was performed to determine ATL monomer purity. Sample was run on an ACQUITY Protein BEH SEC column (200A, 1.7 um, 4.6 mm×150 mm) installed on an ACQUITY UPLC instrument (Waters), using 10 mM phosphate, 1.0 M sodium perchlorate, 5% v/v isopropanol as mobile phase, at a flow rate of 0.3 mL/min, and monitored UV-vis absorbance at 280 nm using an eλ PDA detector (Waters). The analytical SEC result (FIG. 3) indicated 99.6% monomer purity.

Analytical Hydrophobic interaction Chromatography (HIC) was performed to evaluate sample hydrophobicity, purity, and homogeneity (FIG. 4). HIC samples were prepared by diluting ˜100 pg antibody or ATL with 1.5 M ammonium sulfate, then loaded on a TSKgel Butyl NPR column (100 mm×4.6 mm, 2.5 um, Tosoh Bioscience) installed on an ACQUITY UPLC instrument (Waters) using a binary gradient of buffer A (1.5 M ammonium sulfate, 50 mM potassium phosphate) and buffer B (50 mM potassium phosphate, 5% isopropanol) at a flow rate of 0.2 mL/min, and monitored UV-vis absorbance at 280 nm using an eλ PDA detector (Waters).

Liquid chromatography electrospray ionization mass spectrometry (LC-ESI-MS) analysis was performed to determine the drug distribution profile and to calculate the average drug-to-antibody ratio (DAR) (Table 17). Each sample (20 μL at 1 mg/mL) was digested by IdeS (30 minutes at 37° C.) then loaded onto an ACQUITY UPLC Protein BEH C4 column (10K psi, 300A, 1.7 um, 75 um×100 mm). Mass spectrum was acquired on a Waters Synapt G2-Si mass spectrometer. The major peak is DAR 2 species (FIG. 5).

TABLE 17
Average Drug-to-Antibody Ratio (DAR)

| | Modification | DAR by | (Fab)₂ Mass |
|---|---|---|---|
| Antibody | (conjugation) | ESI-MS | Species (m/z) |
| REGN18121 | None | N/A | 97542 |
| REGN18121 | M6324 | 1.9 | 101066 |
| REGN18121 | M6009 | 1.9 | 101471 |
| REGN7438 | None | N/A | 97647 |
| REGN7438 | M6324 | 2.0 | 101171 |
| REGN7438 | M6009 | 1.9 | 101576 |

### Example 16. M6324 Bioconjugation Process for Preparing Antibody-Tethered Ligands (ATLs): REGN7990-M6324, REGN15869-M6324, and REGN18121-M6324

As detailed in the previous Example 15, similar processes for conjugation and purification steps are carried out. Overall process yields are shown in Table 18.

TABLE 18
Antibody-Tethered Ligand (ATL)

| | Lot # | Overall Yield |
|---|---|---|
| REGN7990-M6324 | L25 | 88% |
| REGN15869-M6324 | L22 | 78%* |
| REGN18121-M6324 | L31 | 91% |

*10.7% HMW in the parent mAb

### 16.1 Conjugation Conditions

REGN7990-M6324 (L25) was used as an example to describe M6324 ATL's conjugation, purification, and characterization details. In a 50 mL Falcon tube, 200 mg REGN7990-L10 (71.1 mg/mL in 10 mM Histidine pH 5.8, 1376 nmol, 2.8 mL) was diluted with 4.6 mL BupH buffer pH 7.0; followed by 730 μL of 1 M Tris pH 8 (75 mM in reaction mixture); and 15 eq. linker payload M6324-L7 (40 mg/mL in MQ water, solubilized from M6324-L6) was added. The reaction mixture was warmed to 37° C. before adding 571 μL Millipore MTG (1U/mg Ab, 350 U/mL in BupH buffer pH 7.0). Reaction mixture was incubated at 37° C. for 2.5 hours with gentle mixing.

### 16.2 Purification Procedure

The crude ATL (9.7 mL) was purified by size-exclusion chromatography (Superdex 200 PG 16/60, 120 mL CV, flow rate 1.2 mL/min, eluant: PBS). The monomer fraction was dialyzed (overnight at 4° C.) into formulation buffer then sterile filtered.

TABLE 19
Materials

| | | Catalog # | | |
|---|---|---|---|---|
| Reagent | Supplier | Manufacturer | Lot# | Spec/note |
| Microbial | Millipore- | SAE0159 | SLCK1576 | Lyophilized powder; |
| Transglutaminase | Sigma | | | 1 bottle (500 mg) |
| (mTGase) | | | | dissolved in 6 mL |
| | | | | BupH buffer; pH7.0 |
| Sodium | Sigma- | S9638-1KG | 021M0018V | |
| Phosphate | Aldrich | | | |
| monobasic | | | | |
| monohydrate | | | | |
| Sodium | Sigma- | 71643-1KG | BCBD2439V | |
| Phosphate | Aldrich | | | |
| dibasic dihydrate | | | | |
| Acetic acid | Sigma- | 320099- | SHBC1632V | >99.7% |
| | Aldrich | 500 ML | | |
| Sucrose | Sigma | S9378-5KG | | >99.5% |
| Polysorbate 80 | VWR | BDH7781-2 | 20D3056990 | 500 mL |

TABLE 20
Materials

| | | Catalog # | | |
|---|---|---|---|---|
| General Supply | Supplier | Manuf. | Lot# | Spec/note |
| UltraPure 1M | Invitrogen | 15568-025 | 1580720 | 1000 mL |
| Tris-HC1 pH8.0 | | | | |
| 1M Acetate | AlfaAesar | J60964-AP | S10B526 | 500 mL |
| Buffer, pH5.0 | | | | |
| BupH phosphate | Thermo | 28372 | VF304698 | 0.1M sodium |
| buffered saline | Scientific | | | phosphate, 0.15M |
| | | | | sodium chloride, |
| | | | | pH7.2, when pouch |
| | | | | dissolved in 500 mL |
| | | | | water |
| DPBS (10×) | Gibco | 14200-075 | 1919077 | 500 mL, pH7.1 after |
| | | | | dilution to 1×; Ca |
| | | | | Mg free |
| Dialysis tubing | Sigma | D0405- | | avg. flat width 23 |
| cellulose | | 100FT | | mm (0.9 in.), MWCO |
| membrane | | | | 12400, 99.99% |
| | | | | retention |

### 16.3 Analytical Characterization

UV-vis standard curve for M6324 has not been generated. M3190 extinction coefficient was used to calculate M6324 ATL concentration:

$$\begin{matrix}REGN 7990 - M 6324 ATL concentration = ( A 280 ) / ( 1.42 \times ( 1 + 0.0195 \times DAR ) ) \\ EC 280 ( DAR 2 ) = 1.47 {g}^{- 1} {cm}^{- 1}\end{matrix}$$

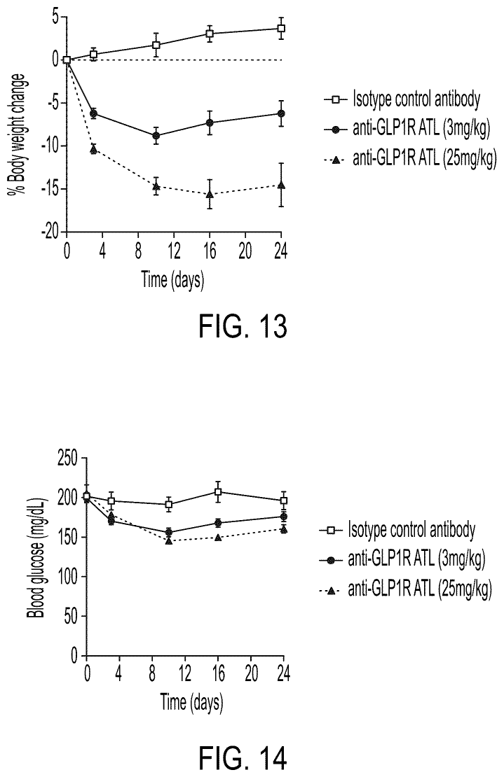

Analytical size exclusion chromatography (SEC) was performed to determine ATL monomer purity. Sample was run on an ACQUITY Protein BEH SEC column (200A, 1.7 um, 4.6 mm×150 mm) installed on an ACQUITY UPLC instrument (Waters), using 10 mM phosphate, 1.0 M sodium perchlorate, 5% v/v isopropanol as mobile phase, at a flow rate of 0.3 mL/min. UV-vis absorbance was monitored at 280 nm. The analytical SEC result (FIG. 13) indicated 98.7% monomer purity.

Liquid chromatography electrospray ionization mass spectrometry (LC-ESI MS) analysis was performed to determine the drug distribution profile and to calculate the average drug-antibody ratio (DAR). Each sample (20 μL at 1 mg/mL) was either deglycosylated by PNGase F enzyme or digested by FabRICATOR enzyme, then loaded onto an ACQUITY UPLC Protein BEH C4 column (10K psi, 300A, 1.7 um, 75 um×100 mm); mass spectrum was acquired on a Waters Synapt G2-Si mass spectrometer. Mass spectrometry data of deglycosylated sample revealed that Lot 25 was a mixture of DAR1, DAR2, DAR3 and DAR4 species. Average LCMS DAR=2.3.

Analytical Hydrophobic Interaction Chromatography (HIC) was performed to compare ATL hydrophobicity, determine the drug distribution profile, and calculate the average DAR. HIC samples were prepared by diluting ˜100 pg antibody or ATL with 1.5 M ammonium sulfate. The samples were loaded on a TSKgel Butyl NPR column (100 mm×4.6 mm, 2.5 um, Tosoh Bioscience) installed on an ACQUITY UPLC instrument (Waters). A binary gradient of buffer A (1.5 M ammonium sulfate, 50 mM potassium phosphate) and buffer B (50 mM potassium phosphate, 5% isopropanol) was used at a flow rate of 0.2 mL/min. UV-vis absorbance was monitored at 280 nm.

REGN7990-M6324 ATL was significantly less hydrophobic than M3190 ATL. HIC also revealed that Lot 25 is a mixture of DAR1 (11%), DAR2 (64%), DAR3 (21%) and DAR4 (4%) species. Average HIC DAR=2.2.

TABLE 21
Antibody-Tethered Ligands (ATL)

| | Lot | Conc. | | | Scale | | Inventory |
|---|---|---|---|---|---|---|---|
| | # | (mg/mL) | DAR | HMW | (gram) | Yield | (gram) |
| REGN7990- | L25 | 15.7 | 2.3 | 1.3% | 0.20 | 88% | 0.110 |
| M6324 | | | | | | | |
| REGN15869- | L22 | 14.3 | 2.0 | 3.2% | 0.20 | 78% | 0.091 |
| M6324 | | | | | | | |
| REGN18121- | L31 | 15.0 | 2.0 | 1.1% | 0.16 | 91% | 0.084 |
| M6324 | | | | | | | |

### Example 17. M6447 Bioconjugation to an GLP1R Antibody: REGN7990-M6447, REGN15869-M6447, and REGN18121-M6447

M6447 was conjugated with three GLP1R antibodies (Table 22) using a similar process as detailed in Example 15. The conjugates were used for in vivo study. The targeted specification was <5% high molecular weight species (HMW).

TABLE 22
Antibody-Tethered Ligands (ATL)

| | Lot | Conc. | | | Endotoxin | Amt. | Overall | Free drug |
|---|---|---|---|---|---|---|---|---|
| | # | (mg/mL) | DAR | HMW | (EU/mg) | (g) | Yield | % (payload) |
| REGN7990- | L26 | 8.95 | 2.1 | 1.2% | 0.3 | 0.073 | 73%* | n.d. |
| M6447 | | | | | | | | |
| REGN15869- | L23 | 9.30 | 2.0 | 0.5% | 0.2 | 0.083 | 83% | n.d. |
| M6447 | | | | | | | | |
| REGN18121- | L32 | 8.73 | 2.0 | 0.4% | 0.1 | 0.083 | 83% | n.d. |
| M6447 | | | | | | | | |

*10.1% HMW in the parent mAb; n.d. not determined.

Overall process yields are shown in Table 23.

TABLE 23
Antibody-Tethered Ligands (ATL)

| | Lot # | Overall Yield |
|---|---|---|
| REGN7990-M6447 | L26 | 73%* |
| REGN15869-M6447 | L23 | 83% |
| REGN18121-M6447 | L32 | 83% |

*10.1% HMW in the parent mAb

### 17.1 Conjugation Conditions

REGN15869-M6447 (L23) was used as an example to describe M6447 ATL's conjugation, purification, and characterization details. In a 15 mL Falcon tube, 100 mg REGN15869-L3 (61.3 mg/mL in 10 mM Histidine pH 5.8, 688 nmole, 1.6 mL) was diluted with 2.1 mL BupH buffer pH 7.0; followed by 362 μL of 1 M Tris pH 8 (75 mM in reaction mixture); and 15 eq. linker payload M6447-L3 (40 mg/mL in MQ water, solubilized from M6447-L1) was added. The reaction mixture was warmed to 37° C. before adding 286 μL Millipore MTG (1U/mg Ab, 350 U/mL in BupH buffer pH 7.0). Reaction mixture was incubated at 37° C. for 4.5 hours with gentle mixing.

### 17.2 Purification Procedure

Crude ATL (4.8 mL) was purified by size-exclusion chromatography (Superdex 200 PG 16/60, 120 mL CV, flow rate 2 mL/min, eluant: PBS). Monomer fraction was dialyzed (overnight at 4° C.) into formulation buffer then sterile filtered.

TABLE 24
Materials

| | | Catalog # | | |
|---|---|---|---|---|
| Reagent | Supplier | Manufacturer | Lot# | Spec/note |
| Microbial | Millipore- | SAE0159 | SLCK1576 | Lyophilized powder; |
| Transglutaminase | Sigma | | | 1 bottle (500 mg) |
| (mTGase) | | | | dissolved in 6 mL |
| | | | | BupH buffer; pH7.0 |
| Sodium | Sigma- | S9638-1KG | 021M0018V | |
| Phosphate | Aldrich | | | |
| monobasic | | | | |
| monohydrate | | | | |
| Sodium | Sigma- | 71643-1KG | BCBD2439V | |
| Phosphate dibasic | Aldrich | | | |
| dihydrate | | | | |
| Acetic acid | Sigma- | 320099- | SHBC1632V | >99.7% |
| | Aldrich | 500ML | | |
| Sucrose | Sigma | S9378-5KG | | >99.5% |
| Polysorbate 80 | VWR | BDH7781-2 | 20D3056990 | 500 mL |

TABLE 25
Materials

| | | Catalog # | | |
|---|---|---|---|---|
| General Supply | Supplier | Manuf. | Lot# | Spec/note |
| UltraPure 1M Tris-HCl | Invitrogen | 15568-025 | 1580720 | 1000 mL |
| pH8.0 | | | | |
| 1M Acetate Buffer, | AlfaAesar | J60964-AP | S10B526 | 500 mL |
| pH5.0 | | | | |
| BupH phosphate | Thermo | 28372 | VF304698 | 0.1M sodium phosphate, 0.15M |
| buffered saline | Scientific | | | sodium chloride, pH7.2, when |
| | | | | pouch dissolved in 500 mL |
| | | | | water |
| DPBS (10×) | Gibco | 14200-075 | 1919077 | 500 mL, pH7.1 after dilution to |
| | | | | 1×; Ca Mg free |
| Dialysis tubing | Sigma | D0405-100FT | | avg. flat width 23 mm (0.9 in.), |
| cellulose membrane | | | | MWCO 12400, 99.99% |
| | | | | retention |

### 17.3 Analytical Characterization

UV-vis standard curve for M6447 has not been generated. M3190 extinction coefficient was used to calculate M6447 ATL concentration:

$$\begin{matrix}REGN 15869 - M 6447 ATL concentration = ( A 280 ) / ( 1.42 \times ( 1 + 0.0195 \times DAR ) ) \\ EC 280 ( DAR 2 ) = 1.47 {g}^{- 1} {cm}^{- 1}\end{matrix}$$

Analytical size exclusion chromatography (SEC) was performed to determine ATL monomer purity (FIG. 18). Sample was run on an ACQUITY Protein BEH SEC column (200A, 1.7 um, 4.6 mm×150 mm) installed on an ACQUITY UPLC instrument (Waters), using 10 mM phosphate, 1.0 M sodium perchlorate, 5% v/v isopropanol as mobile phase, at a flow rate of 0.3 mL/min. UV-vis absorbance was monitored at 280 nm. The analytical SEC result indicated 98.7% monomer purity.

Liquid chromatography electrospray ionization mass spectrometry (LC-ESI MS) analysis was performed to determine the drug distribution profile and to calculate the average drug-antibody ratio (DAR). Each sample (20 μL at 1 mg/mL) was either deglycosylated by PNGase F enzyme and/or digested by FabRICATOR enzyme, then loaded onto an ACQUITY UPLC Protein BEH C4 column (10K psi, 300A, 1.7 um, 75 um×100 mm). Mass spectrum was acquired on a Waters Synapt G2-Si mass spectrometer.

Mass spec data for deglycosylated ATL revealed that Lot 23 was a mixture of DARI, DAR2 and DAR3 species. Average LCMS DAR=2.2.

Mass spec data for the deglycosylated and IdeS digested ATL revealed a little drug loading on Fc; average LCMS DAR=2.0.

Analytical Hydrophobic interaction Chromatography (HIC) was performed to compare ATL hydrophobicity, determine the drug distribution profile and calculate the average DAR. HIC samples were prepared by diluting ˜100 pg antibody or ATL with 1.5 M ammonium sulfate, then loaded on a TSKgel Butyl NPR column (100 mm×4.6 mm, 2.5 um, Tosoh Bioscience) installed on an ACQUITY UPLC instrument (Waters) using a binary gradient of buffer A (1.5 M ammonium sulfate, 50 mM potassium phosphate) and buffer B (50 mM potassium phosphate, 5% isopropanol) at a flow rate of 0.2 mL/min. UV-vis absorbance was monitored at 280 nm.

ATL REGN15869-M6447 was significantly less hydrophobic than that of REGN15869-M3190 ATL. HIC HIC also revealed Lot 23 is a mixture of DAR1 (8%) and DAR2 (92%). Average HIC DAR=2.0

TABLE 26
Antibody-Tethered Ligands (ATL)

| | Lot | Conc. | | | Scale | | Inventory |
|---|---|---|---|---|---|---|---|
| | # | (mg/mL) | DAR | HMW | (gram) | Yield | (gram) |
| REGN7990- | L26 | 8.95 | 2.1 | 1.2% | 0.10 | 73% | 0.055 |
| M6447 | | | | | | | |
| REGN15869- | L23 | 9.30 | 2.0 | 0.5% | 0.10 | 83% | 0.065 |
| M6447 | | | | | | | |
| REGN18121- | L32 | 8.73 | 2.0 | 0.4% | 0.10 | 83% | 0.066 |
| M6447 | | | | | | | |

### Example 18—In Vitro Activities of Linker-Payloads and their ATLs Using CRE-Luc Assay, β-Arrestin 1, and β-Arrestin 2 Recruitment Assays 18.1 Cell Lines and Growth Media

Cell lines and growth media used in this example are described in Table 27 below.

TABLE 27
Cell Lines and Growth Media

| Name | Cell Lines | Growth Media |
|---|---|---|
| HEK293/CRE- | HEK293/FSC11/pCDNA3.1 + G | DMEM + 10% FBS + 1× Penicillin- |
| Luc/hGLPIR | LP1R + 1 nM GLP1 (ACL6822) | Streptomycin + 500 ug/ml G418 |
| HEK293/b- | HEK293/B-Arrestin-EA clone | DMEM + 10% FBS + 1× Penicillin- |
| arrestin 1/hGLP1R | 7/hGLP1R-Prolink1 high sorted | Streptomycin + 100 ug/ml |
| | (ACL21690) | Hygromycin + 1 ug/ml Puromycin |
| HEK293/b- | HEK293/Barrestin clone | DMEM + 10% FBS + 1× Penicillin- |
| arrestin2/hGLP1R | 2C6/hGLP1R-Prolink1 high | Streptomycin + 100 ug/ml |
| | sorted (ACL21687) | Hygromycin + 1 ug/ml Puromycin |

Reagents used in this example are described in Table 28 below.

TABLE 28
Reagents used in the assays

| Reagent Name | Supplier, Catalog Number |
|---|---|
| Dulbecco's Modified Eagle's Medium (DMEM) | Gibco, #11995065 |
| Fetal Bovine Serum (FBS) | Seradigm, #1500-500 |
| Penicillin-Streptomycin (100×) | Gibco, #15140122 |
| Geneticin Selective Antibiotic (G418) | Gibco, #11811098 |
| Hygromycin B | InvivoGen, #ant-hg-5 |
| Puromycin | Sigma-Aldrich, #P8833 |
| Dulbecco's phosphate-buffered saline (PBS), no calcium, | Gibco, #14190144 |
| no magnesium | |
| T75 cell culture treated flask | Corning, #430641U |
| 0.025% trypsin and 0.75 mM EDTA (1×) | Sigma-Aldrich, #SM-2004-C |
| Dimethylsulfoxide (DMSO), cell culture tested | ATCC, #4-X-5 |
| Opti-MEM I Reduced Serum Medium | Gibco, #31985070 |
| Masterblock, 384 Well, PP, Deep Well, V-Bottom, | Greiner Bio-One #781271 |
| Natural, Sterile | |
| Luminescence assay plates, 384-Well, Cell Culture- | Falcon #353988 |
| Treated, Flat-Bottom Microplate | |
| VIAFLO 384 | Integra |
| TopSeal-A PLUS | Perkin Elmer, #6050185 |
| ONE-Glo Luciferase Assay System | Promega, #E6130 |
| PathHunter Detection Reagent | Eurofins DiscoverX, #93-0001 |
| EnVision Plate Reader | Perkin Elmer |

### 18.2 Procedure

Glucagon-like peptide 1 receptor (GLP1R) is a member of the secretin family (Class B) of G protein-coupled receptors (GPCRs). Upon binding of its ligand (GLP-1), GLP1R initiates a downstream signaling cascade through Gas G-proteins that raises intracellular cyclic AMP (cAMP) levels, which leads to the transcriptional regulation of genes (Donnelly D., “The Structure and Function of the Glucagon-Like Peptide-1 Receptor and its Ligands,” British Journal of Pharmacology: 166:27-41 (2012), which is incorporated by reference in its entirety). GLP-1 binding also results in b-arrestin 1 and b-arrestin 2 recruitment to GLP1R.

To test the activity of GLP1R agonist linker-payloads (LPs) and anti-GLP1R antibody-tethered ligands (ATLs) of the invention, a cell-based cAMP responsive luciferase reporter assay was developed. To generate the assay cell line, the firefly luciferase gene was placed under the control of four copies of a cAMP response element (4×CRE) located upstream of a minimal promoter and transfected into HEK293 cells and referred to herein as HEK293/CRE-Luc cells. HEK293/CRE-Luc cells were then engineered to express full-length human GLP1R (GenBank: AAI12127.1) (HEK293/CRE-Luc/hGLP1R).

To test the recruitment of b-arrestin 1 and b-arrestin 2 to GLP1R upon receptor activation, two enzyme fragment complementation assays were developed. To generate the assay cell lines, b-arrestin 1 fused to the b-galactosidase (b-gal) reporter fragment called enzyme acceptor (EA) (i.e., b-arrestin1-EA) or b-arrestin 2 fused to the b-gal reporter fragment EA (i.e., b-arrestin2-EA) were transduced into HEK293 cells using PathHunter b-Arrestinl Retroparticles (DiscoverX, #83-OT-TB-N-01) or PathHunter b-Arrestin2 Retroparticles (DiscoverX, #93-1087), respectively. The full-length human GLP1R (NP_002053.3) with a C-terminus fusion to a small b-gal fragment called enzyme donor or ProLink (PK) (i.e., hGLP1R-PK) was then engineered into HEK293/b-arrestinl-EA cells and HEK293/b-arrestin2-EA cells to generate the cell lines referred herein as HEK293/b-arrestin1/hGLP1R and HEK293/b-arrestin2/hGLP1R. Recruitment of b-arrestin 1 or b-arrestin 2 to GLP1R in these reporter cell lines brings both b-gal fragments (EA and PK) into close proximity, leading to reconstitution of a fully functional enzyme capable of hydrolyzing a substrate and generating a chemiluminescent signal.

For all assays, cells were seeded into 384-well plates (Falcon #353988) at 6,000 cells/well in assay media (Opti-MEM, 1% FBS, 1×Penicillin-Streptomycin) and incubated overnight at 37° C. Serial dilutions of the anti-GLP1R ATLs and non-binding control ATLs were performed in assay media. Serial dilutions of linker-payloads (LPs) were performed in 100% DMSO (ATCC, #4-X-5), followed by 1:100 dilution in assay media. Test articles were added to cells (1:5 dilution) using VIAFLO 384 (Integra) with the last well in each serial dilution series serving as a blank control containing only assay media for ATLs or assay media with 0.2% DMSO for LPs. The blank was plotted as a continuation of the serial dilution.

For the CRE-Luc assay, after a 5-hours incubation at 37° C., luciferase activity was determined by addition of ONE-Glo reagent (Promega, #E6130) followed by measurement of relative light units (RLUs) on EnVision Plate Reader (Perkin Elmer).

For b-arrestin 1 and b-arrestin 2 recruitment assays, after a 1-hour incubation at 37° C., PathHunter Detection Reagent (Eurofins DiscoverX, #93-0001) was added to the wells followed by a 1-hour incubation at room temperature. RLUs were measured on EnVision Plate Reader (Perkin Elmer).

EC₅₀ values for luminescence assays were determined using a four-parameter logistic equation over a 12-point dose response curve (GraphPad Prism). The maximum signal relative to GLP-1 (Eₘₐₓ (% GLP-1)) was calculated using the following equation:

$${E}_{max} ( \% GLP - 1 ) = ( {RLU}_{max test article} - {RLU}_{blank} ) / ( {RLU}_{max GLP - 1} - {RLU}_{blank} ) \times 100 \qquad ( Eq . 1 )$$

### 18.3 Results Summary and Conclusions

GLP1R activation by ligand binding promotes a signaling cascade through Gas G-protein that raises intracellular cyclic AMP (cAMP) levels. In addition, activated GLP1R can recruit b-arrestins, leading to receptor internalization and/or additional signaling pathways (Jones B., “The Therapeutic Potential of GLP-1 Receptor Biased Agonism,” British Journal of Pharmacology 179:492-510 (2022); Donnelly D, The structure and function of the glucagon-like peptide-1 receptor and its ligands, British Journal of Pharmacology, 2012: 166:27-41, PMID 21950636, which is incorporated by reference in its entirety). Ligands can induce distinct cellular outcomes through the same receptor by preferential signaling through G-proteins or b-arrestins—a phenomenon designated biased signaling.

GLP1R agonist linker payloads (M3190, M6009, M6447, and M6324) were conjugated to anti-hGLP1R antibodies (REGN7990, REGN15869, and REGN18121) and the non-binding isotype control antibody (REGN7438) via N-terminal light chain glutamine tag (Q tag). Test articles were tested for biased signaling by evaluating activity in a cAMP reporter assay which over expresses human GLP1R (using the HEK293/CRE-Luc/hGLP1R cells) and in b-arrestin 2 recruitment assay which over expresses human GLP1R (using HEK293/b-arrestin2/hGLP1R cells).

The endogenous GLP1R ligand, GLP-1 (7-36) amide (referred to as GLP-1), increased CRE-dependent luciferase reporter activity in HEK293/CRE-Luc/hGLP1R cells with an EC₅₀ values ranging from 0.185 of 0.555 nM (from three independent experiments), and recruited b-arrestin 2 in HEK293/b-arrestin2/hGLP1R cells with an EC₅₀ value ranging from 22.5 to 38.1 nM (from two independent experiments. See Table 29). The three tested linker payloads exhibited distinct in vitro properties: M3190 increased CRE-dependent luciferase reporter activity with an EC₅₀ value of 576 pM, and recruited b-arrestin 2 with an EC₅₀ value of 35.1 nM and Eₘₐₓ relative to GLP-1 of 128.4%; M6009, a metabolite of M3190, showed a comparable activities as that of M3190; M6324 increased CRE-dependent luciferase reporter activity with an EC₅₀ value of 293 nM, and recruited very low levels of b-arrestin 2 (EC₅₀>1 mM and Eₘₐₓ relative to GLP-1 of 0.7%); M6447 was a very weak activator of CRE-dependent luciferase reporter activity and recruited very low levels of b-arrestin 2 (EC₅₀>1 mM for both assays).

The three tested anti-GLP1R-M3190 ATLs (REGN7990-M3190, REGN15869-M3190, and REGN18121-M3190) increased CRE-dependent luciferase reporter activity in HEK293/CRE-Luc/hGLP1R cells with similar potencies (EC₅₀ values ranging from 165 pM to 183 pM) and Eₘₐₓ relative to GLP-1 ranging from 93.7% to 96.4%. b-arrestin 2 recruitment was also similar between the anti-GLP1R-M3190 ATLs (EC₅₀ values ranging from 15.3 nM to 18.9 nM) and Eₘₐₓ relative to GLP-1 ranging from 99.7% to 136.0%. The ATL pair of M6009, e.g. REGN18121-M6009 and REGN7348-M6009, behaved more or less in the same way as the ATLs of M3190 (Table 29).

The three tested anti-GLP1R-M6324 ATLs (REGN7990-M6324, REGN15869-M6324, and REGN18121-M6324) increased CRE-dependent luciferase reporter activity in HEK293/CRE-Luc/hGLP1R cells with similar potencies (EC₅₀ values ranging from 16.3 pM to 21.4 pM) and Eₘₐₓ relative to GLP-1 ranging from 97.0% to 98.1%. b-arrestin 2 recruitment was also similar between the anti-GLP1R-M6324 ATLs (EC₅₀ values ranging from 8.6 nM to 10.4 nM) and Eₘₐₓ relative to GLP-1 ranging from 46.7% to 54.9% (Table 29).

The three tested anti-GLP1R-M6447 ATLs (REGN7990-M6447, REGN15869-M6447, and REGN18121-M6447) increased CRE-dependent luciferase reporter activity in HEK293/CRE-Luc/hGLP1R cells with similar potencies (EC₅₀ values ranging from 31.2 pM to 34.3 pM) and Eₘₐₓ relative to GLP-1 ranging from 95.2% to 98.0%. b-arrestin 2 recruitment was also similar between the anti-GLP1R-M6447 ATLs (EC₅₀ values ranging from 0.75 nM to 1.01 nM) and Eₘₐₓ relative to GLP-1 ranging from 1.2% to 1.7% (Table 20).

The non-binding control ATLs (REGN7438-M3190, REGN7438-M6324, and REGN7438-M6447) exhibited reduced potencies in CRE-dependent luciferase reporter activity than the corresponding anti-GLP1R ATLs: EC₅₀ values of 4.62 nM, >1 mM, and 1.09 nM for REGN7438-M3190, REGN7438-M6324, and REGN7438-M6447, respectively (Table 29).

These results show that anti-GLP1R ATLs of GLP1 agonists linker-payloads M3190, M6324, M6009, and M6447 can potently activate human GLP1R while recruiting different levels of b-arrestin 2, suggesting a differential impact in downstream signaling.

The non-binding isotype control ATLs tested (REGN7438-M3190 and REGN7438-M6009) can still promote some GLP1R activation due to the presence of GLP1R agonist LPs but have reduced potencies when compared to the respective anti-GLP1R ATL in the CRE-Luc assay and in b-arrestin recruitment assays (Table 29).

TABLE 29
CRE-dependent reporter activity and b-Arrestin2 recruitment induced by ATLs and GLPIR
agonists in HEK293/CRE-Luc/hGLPIR cells and HEK293/b-arrestin2/hGLPIR cells.

| | | HEK293/ | | HEK293/ | | |
|---|---|---|---|---|---|---|
| | | CRE-Luc/hGLPIR | | b-arrestin2/hGLPIR | | |
| Test | | EC50 | Eₘₐₓ | EC50 | Eₘₐₓ | |
| article | LP | (M) | (% GLP-1) | (M) | (% GLP-1) | Experiment |
| GLP-1 (7-36) | | 5.55E−10 | 100.0 | 3.81E−08 | 100.0 | A |
| amide | | 4.23E−10 | 100.0 | 2.25E−08 | 100.0 | B |
| | | 1.85E−10 | 100.0 | NT | NT | C * |
| M3190 | | 5.76E−10 | 96.3 | 3.51E−08 | 128.4 | A |
| M6009 | | 4.64E−10 | 92.1 | NT | NT | B |
| M6324 | | 2.93E−07 | 91.6 | >1.00E−06 | 0.7 | A |
| M6447 | | >1.00E−06 | 9.8 | >1.00E−06 | 0.3 | A |
| REGN7990- | M3190 | 1.83E−10 | 96.2 | 1.89E−08 | 136.0 | A |
| M3190 | | | | | | |
| (GLP1R-ATL) | | | | | | |
| REGN15869- | M3190 | 1.72E−10 | 93.7 | 1.53E−08 | 99.7 | A |
| M3190 | | | | | | |
| (GLPIR-ATL) | | | | | | |
| REGN18121- | M3190 | 1.65E−10 | 96.4 | 1.83E−08 | 114.6 | A |
| M3190 | | | | | | |
| (GLPIR-ATL) | | | | | | |
| REGN7438- | M3190 | 4.62E−09 | 94.6 | >1.00E−06 | 66.6 | B |
| M3190 | | | | | | |
| (isotype-ATL) | | | | | | |
| REGN18121- | M6009 | 1.21E−10 | 93.8 | 1.07E−08 | 84.4 | A |
| M6009 | | | | | | |
| (GLPIR-ATL) | | | | | | |
| REGN7438- | M6009 | 3.49E−08 | 94.7 | >1.00E−06 | ND | A |
| M6009 | | | | | | |
| (isotype-ATL) | | | | | | |
| REGN7990- | M6324 | 2.14E−11 | 98.0 | 8.89E−09 | 46.7 | A |
| M6324 | | | | | | |
| (GLP1R-ATL) | | | | | | |
| REGN15869- | M6324 | 1.94E−11 | 98.1 | 1.04E−08 | 54.9 | A |
| M6324 | | | | | | |
| (GLPIR-ATL) | | | | | | |
| REGN18121- | M6324 | 1.63E−11 | 97.0 | 8.60E−09 | 48.3 | A |
| M6324 | | | | | | |
| (GLPIR-ATL) | | | | | | |
| REGN7438- | M6324 | >1.00E−06 | 52.3 | >1.00E−06 | 0.5 | A |
| M6324 | | | | | | |
| (isotype-ATL) | | | | | | |
| REGN7990- | M6447 | 3.35E−11 | 98.0 | 7.92E−10 | 1.7 | A |
| M6447 | | | | | | |
| (GLPIR-ATL) | | | | | | |
| REGN15869- | M6447 | 3.43E−11 | 95.2 | 1.01E−09 | 1.2 | A |
| M6447 | | | | | | |
| (GLPIR-ATL) | | | | | | |
| REGN18121- | M6447 | 3.12E−11 | 97.5 | 7.50E−10 | 1.4 | A |
| M6447 | | | | | | |
| (GLP1R-ATL) | | | | | | |
| REGN7438- | M6447 | 1.09E−09 | 110.8 | NT | NT | C |
| M6447 | | | | | | |
| (isotype-ATL) | | | | | | |

> = EC₅₀ values could not be determined with accuracy because luminescence signal did not reach saturation within the tested concentration range. EC₅₀ is reported as greater than the highest tested concentration. NT, not tested.
*GLP-1 (7-36) amide included in A, B, and C experiments with similar results. Experiments A, B, and C represent independent experiments.

### Example 19—In Vivo Efficacy Evaluation

19.1. In Vivo Efficacy Studies of Antibody-Tethered Ligands: The Effects of Anti-GLP1R Antibody-Tethered-Ligand REGN18121-M6324 and Comparison with that of the Isotype Control (REGN7438-M6324) on Body Weight and Blood Glucose in Diet-Induced Obese Mice

To determine body weight and blood glucose lowering effects of an anti-GLP1R antibody-tethered-ligand (ATL) of the invention in obese animals, mice homozygous for the expression of human GLP1R in place of mouse GLP1R (referred to as GLP1R humanized mice) were placed on high-fat diet (60% kcal % fat) for 5 months. Twenty, 7-month-old male, GLP1R humanized mice were stratified into three groups of six or seven mice, based on their day 0 body weights. After the stratification, each group was subcutaneously administered with anti-GLP1R ATL at 3 mg/kg or 25 mg/kg or isotype control antibody (referred to as Control) at 25 mg/kg on day 0.

On days three, ten, sixteen, and twenty-four post administration, body weights of the animals were recorded, and their blood glucose levels were measured with a handheld glucometer. Mean±SEM of percent changes in body weight from day 0 at each time point was calculated for each group and are shown in Table 30. Mean±SEM of blood glucose levels at each time point was calculated for each group and are shown in Table 31. Statistical analyses were performed by two-way ANOVA followed by Bonferroni post-hoc tests, comparing the Control group to the other two groups.

TABLE 30
Effects of GLPIR ATL on Percent Body Weight
Changes in Obese GLPIR Humanized Mice

| | | | Anti-GLPIR ATL | | Anti-GLPIR ATL | |
|---|---|---|---|---|---|---|
| | Isotype | | REGN18121-M6324 | | REGN18121-M6324 | |
| Time | control antibody | | (3 mg/kg) | | (25 mg/kg) | |
| (day) | Mean | SEM | Mean | SEM | Mean | SEM |
| 0 | 0 | 0 | 0 | 0 | 0 | 0 |
| 3 | 0.7 | 0.8 | −6.2**** | 0.6 | −10.3**** | 0.5 |
| 10 | 1.7 | 1.4 | −8.8*** | 1.0 | −14.7**** | 1.0 |
| 16 | 3.1 | 0.9 | −7.3*** | 1.4 | −15.6**** | 1.7 |
| 24 | 3.7 | 1.3 | −6.2*** | 1.5 | −14.5*** | 2.5 |

***P < 0.001,
****P < 0.0001, compared to the isotype control antibody group.

TABLE 31
Effects of ATL REGN18121-M6324_on Blood
Glucose Levels in Obese GLP1R Humanized Mice

| | | | Anti-GLPIR | | Anti-GLPIR | |
|---|---|---|---|---|---|---|
| | Isotype | | mAb ATL | | mAb ATL | |
| Time | control antibody | | (3 mg/kg) | | (25 mg/kg) | |
| (day) | Mean | SEM | Mean | SEM | Mean | SEM |
| 0 | 202 | 4 | 200 | 6 | 205 | 11 |
| 3 | 196 | 11 | 170 | 5 | 178 | 4 |
| 10 | 191 | 9 | 156* | 6 | 145** | 3 |
| 16 | 207 | 13 | 168 | 5 | 150* | 2 |
| 24 | 196 | 11 | 176 | 6 | 161* | 5 |

*P < 0.05,
**P < 0.01, compared to the isotype control antibody group.

Body weights and blood glucose levels were stable in animals administered with the Control, with nominal handling (i.e., bleeding, cage changes) related fluctuations. In animals administered with anti-GLP1R ATL at either dosage, significant weight loss was observed at all post-administration timepoints. Significant glucose reductions were observed on days 10, 16, and 24 with 25 mg/kg, and on day 10 with 3 mg/kg anti-GLP1R ATL.

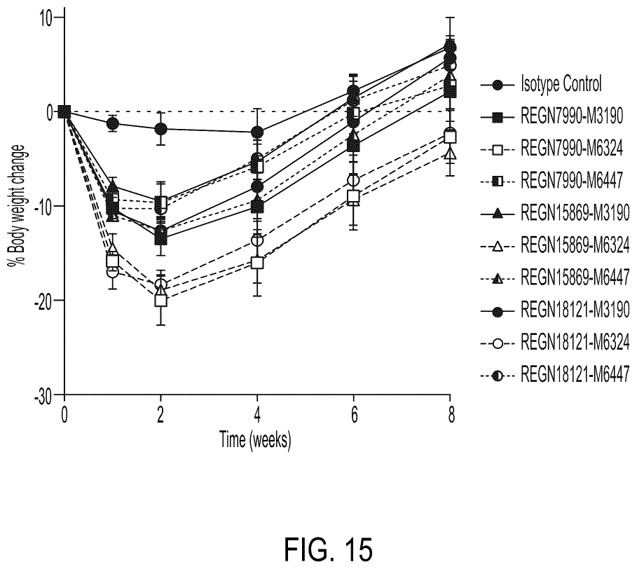

The efficacy studies results are further detailed in FIGS. 13 and 14. In conclusion, single administration of anti-GLP1R ATL of the invention leads to long-term weight and glucose lowering in obese animals.

19.2. Comparison of In Vivo Efficacy of a selected group of Antibody-Tethered Ligands: In another set of efficacy studies, the following group of ATLs were evaluated by following a similar protocol as shown in Table 32.

TABLE 32
ATLs used in the second set of in vivo efficacy studies

| | Test Articles/Reagents |
|---|---|
| 1 | REGN1945 (isotype control antibody) |
| 2 | REGN7990-M3190 |
| 3 | REGN7990-M6324 |
| 4 | REGN7990-M6447 |
| 5 | REGN15869-M3190 |
| 6 | REGN15869-M6324 |
| 7 | REGN15869-M6447 |
| 8 | REGN18121-M3190 |
| 9 | REGN18121-M6324 |
| 10 | REGN18121-M6447 |

To determine body weight lowering effects of anti-GLP1R antibody-tethered-ligands (ATLs) of the invention in obese animals, mice homozygous for the expression of human GLP1R in place of mouse GLP1R (referred to as GLP1R humanized mice) were placed on high-fat diet (60% kcal % fat) for 4 months. Sixty-five, 6-month-old male, GLP1R humanized mice were stratified into ten groups of five to seven mice, based on their day 0 body weights. After the stratification, each group was subcutaneously administered with one of nine an anti-GLP1R ATLs or an isotype control antibody (referred to as Control) at 25 mg/kg on day 0.

At weeks one, two, four, six and eight post administration, body weights of the animals were recorded. Mean±SEM of percent changes in body weight from day 0 at each time point was calculated for each group and are shown in Table 33. Statistical analyses were performed by two-way ANOVA followed by Bonferroni post-hoc tests, comparing the Control group to the other nine groups.

TABLE 33
Effects of GLPIR ATL on percent body weight
changes in obese GLPIR humanized mice

| | | Time (week) | | | | |
|---|---|---|---|---|---|---|
| | | 1 | 2 | 4 | 6 | 8 |
| Isotype | Mean | −1.3 | −1.8 | −2.2 | 2.2 | 6.8 |
| control | SEM | 0.9 | 1.7 | 2.5 | 1.8 | 1.2 |
| antibody | | | | | | |
| REGN7990- | Mean | −10.2*** | −13.5** | −10.1 | −3.6 | 2.1 |
| M3190 | SEM | 1.2 | 1.8 | 2.9 | 3.1 | 3.2 |
| REGN7990- | Mean | −15.9**** | −20.0*** | −16.0** | −8.9* | −2.7* |
| M6324 | SEM | 1.1 | 2.6 | 3.5 | 3.6 | 2.8 |
| REGN7990- | Mean | −9.3** | −9.6* | −5.8 | −0.2 | 2.7 |
| M6447 | SEM | 1.4 | 2.2 | 3.6 | 4.0 | 4.7 |
| REGN15869- | Mean | −7.9*** | −9.5* | −5.3 | 1.4 | 7.2 |
| M3190 | SEM | 0.9 | 1.9 | 2.3 | 2.4 | 2.8 |
| REGN15869- | Mean | −14.5**** | −18.9**** | −15.7** | −9.3** | −4.4** |
| M6324 | SEM | 1.6 | 1.6 | 2.4 | 2.7 | 2.4 |
| REGN15869- | Mean | −11.0**** | −12.6** | −9.4 | −2.5 | 3.8 |
| M6447 | SEM | 0.4 | 1.4 | 2.3 | 2.8 | 3.6 |
| REGN18121- | Mean | −10.5**** | −12.6** | −8.0 | −1.1 | 5.7 |
| M3190 | SEM | 0.9 | 1.2 | 1.8 | 1.3 | 1.2 |
| REGN18121- | Mean | −17.0**** | −18.3**** | −13.6** | −7.3* | −2.3* |
| M6324 | SEM | 1.8 | 1.5 | 2.3 | 2.7 | 2.6 |
| REGN18121- | Mean | −10.2**** | −10.3** | −5.0 | 1.2 | 4.9 |
| M6447 | SEM | 0.8 | 0.9 | 1.5 | 2.0 | 2.7 |

*P < 0.05,
**P < 0.01,
***P < 0.001,
****P < 0.0001, compared to the isotype control antibody group.

Body weights were stable in animals administered with the Control, with nominal handling (i.e., cage changes) related fluctuations. In animals administered with REGN7990-M6324, REGN15869-M6324 and REGN18121-M6324, significant reductions in body weight were observed at all post-administration timepoints. In animals administered with REGN7990-M3190, REGN7990-M6447, REGN15869-M3190, REGN15869-M6447, REGN18121-M3190, and REGN18121-M6447, significant reductions in body weight were observed at weeks 1 and 2 post-administration, but other time points did not reach significance. Effects of GLP1R ATLs on percent body weight changes in obese GLP1R humanized mice are depicted in FIG. 15.

In conclusion, single administration of anti-GLP1R ATLs of the invention leads to long-term weight lowering in obese animals.

### 19.3. Comparison of the Effects of a Selected Group of Anti-GLP1R Antibody-Tethered-Ligands on Body Weight and Fasting Glucose in Obese and Diabetic Cynomolgus Monkeys

TABLE 34
Test Articles/Reagent IDs:

| | Test Articles/Reagents |
|---|---|
| 1 | REGN7990-M3190 |
| 2 | REGN7990-M6324 |
| 3 | REGN7990-M6447 |
| 4 | REGN15869-M6324 |
| 5 | REGN15869-M6447 |

To determine body weight and plasma glucose lowering effects of anti-GLP1R antibody-tethered-ligands (ATLs) of the invention in obese and diabetic primates, forty-six aged, male cynomolgus monkeys were pre-screened for the presence of obesity and diabetes. In the non-human primates (NHPs), obesity was defined as BMI of 40 kg/m² or above and body weight of 8 kg or above, whereas diabetes was defined as fasting plasma glucose level of 80 mg/dL or above. Twenty-four monkeys meeting the criteria were stratified into five groups of four or five monkeys, based on their baseline body weights. One of the five ATLs were assigned to each group for seven weeks of subcutaneous administration, starting with three weeks of step-up doses and ending with four weeks of a target dose. Each ATL was administered weekly at 0.1 mg/kg on study Day 1, 0.5 mg/kg on Day 8, 2 mg/kg on Day 15, and 6 mg/kg on Days 22, 29, 36, and 43.

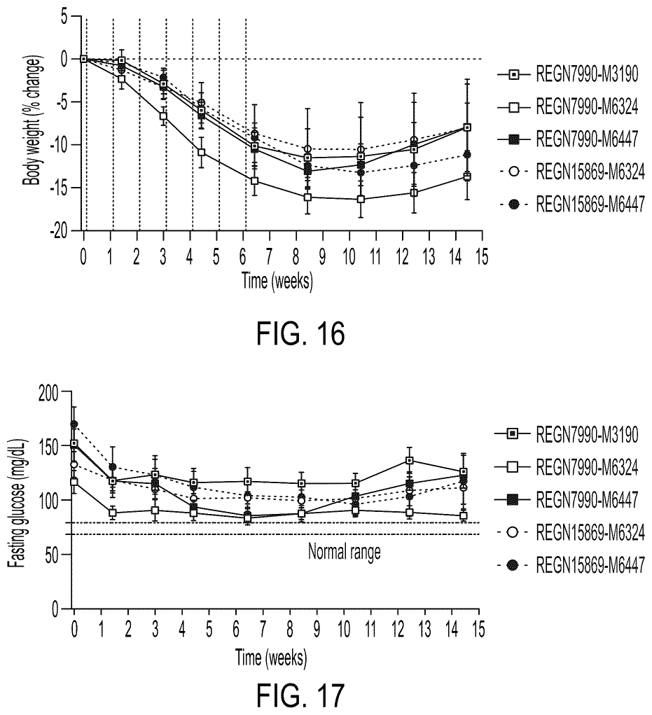

On Days 10, 21, 31, 45, 59, 73, 87 and 101, body weights of the animals were recorded, and their fasting plasma glucose levels were measured using a Roche C311 biochemical analyzer. Mean±SEM of percent changes in body weight from Day −2 at each time point was calculated for each group and are shown in Table 35. Mean±SEM of fasting glucose levels at each time point was calculated for each group and are shown in Table 36. Statistical analyses were performed by two-way ANOVA followed by Bonferroni post-hoc tests, comparing Day 10 or after timepoint values to the baseline values within each group. Effects of GLP1R ATLs on percent body weight changes in NHPs are depicted in FIGS. 16-17.

In animals administered with REGN7990-M6324, REGN7990-M6447 and REGN15869-M6447, significant reductions in body weight were observed at multiple timepoints between Days 31 and 87, which is 44 days post last administration.

In animals administered with REGN7990-M6324, fasting glucose lowering to the upper limit of normal range (between 69 and 79 mg/dL) was observed throughout the study. However, the reductions did not reach statistical significance, likely due to low starting values of the group. In animals administered with REGN799O-M6447 and REGN15869-M6447, significant glucose reductions were observed at two and three timepoints, respectively.

In conclusion, weekly administration of anti-GLP1R ATLs of the invention leads to long-term weight and glucose lowering in obese and diabetic NHPs.

TABLE 35
Effects of GLPIR ATL on percent body weight changes in obese and diabetic NHPs

| | | Time (days) | | | | | | | | |
|---|---|---|---|---|---|---|---|---|---|---|
| Compound | | −2 | 10 | 21 | 31 | 45 | 59 | 73 | 87 | 101 |
| REGN7990- | Mean | 0 | −0.2 | −2.9 | −6.0 | −10.1 | −11.5 | −11.3 | −10.5 | −8.0 |
| M3190 | SEM | 0 | 1.2 | 1.8 | 2.1 | 2.7 | 3.4 | 4.6 | 5.5 | 5.6 |
| REGN7990- | Mean | 0 | −2.3 | −6.7 | −10.9 | −14.2* | −16.1* | −16.4* | −15.6 | −13.7 |
| M6324 | SEM | 0 | 1.2 | 1.1 | 1.8 | 1.7 | 1.9 | 2.1 | 2.4 | 2.7 |
| REGN7990- | Mean | 0 | −0.8 | −3.2 | −6.6 | −10.5* | −13.1* | −12.3 | −10.0 | −7.9 |
| M6447 | SEM | 0 | 0.6 | 0.8 | 1.6 | 1.9 | 2.0 | 2.5 | 2.6 | 2.8 |
| REGN15869- | Mean | 0 | −1.3 | −3.3 | −5.1 | −8.7 | −10.5 | −10.5 | −9.4 | −8.0 |
| M6324 | SEM | 0 | 0.9 | 1.3 | 2.4 | 3.4 | 4.7 | 5.5 | 5.4 | 5.1 |
| REGN15869- | Mean | 0 | −0.4 | −2.1 | −5.8* | −9.2* | −12.4** | −13.2** | −12.4* | −11.1 |
| M6447 | SEM | 0 | 0.4 | 0.8 | 1.1 | 1.1 | 1.4 | 1.5 | 2.2 | 3.1 |

*P < 0.05,
**P < 0.01, compared to the day −2 values for each group.

TABLE 36
Effects of GLPIR ATL on fasting glucose levels in obese and diabetic NHPs

| | | Time (days) | | | | | | | | |
|---|---|---|---|---|---|---|---|---|---|---|
| Compound | | −2 | 10 | 21 | 31 | 45 | 59 | 73 | 87 | 101 |
| REGN7990- | Mean | 152 | 118 | 123 | 116 | 117 | 115 | 115 | 136 | 126 |
| M3190 | SEM | 17 | 11 | 14 | 10 | 13 | 10 | 9 | 12 | 15 |
| REGN7990- | Mean | 117 | 88 | 91 | 88 | 83 | 88 | 91 | 89 | 86 |
| M6324 | SEM | 11 | 6 | 10 | 7 | 6 | 6 | 6 | 6 | 5 |
| REGN7990- | Mean | 151 | 117** | 115 | 94 | 86 | 88* | 104 | 115 | 123 |
| M6447 | SEM | 15 | 15 | 14 | 6 | 7 | 8 | 6 | 6 | 2 |
| REGN15869- | Mean | 133 | 118 | 110 | 101 | 102 | 99 | 101 | 109 | 111 |
| M6324 | SEM | 12 | 10 | 9 | 10 | 6 | 10 | 16 | 17 | 15 |
| REGN15869- | Mean | 170 | 130* | 123 | 112 | 104 | 103 | 96** | 103* | 117 |
| M6447 | SEM | 16 | 19 | 18 | 17 | 11 | 10 | 10 | 18 | 25 |

*P < 0.05,
**P < 0.01, compared to the day −2 values for each group.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A compound having a structure of Formula (P-I): 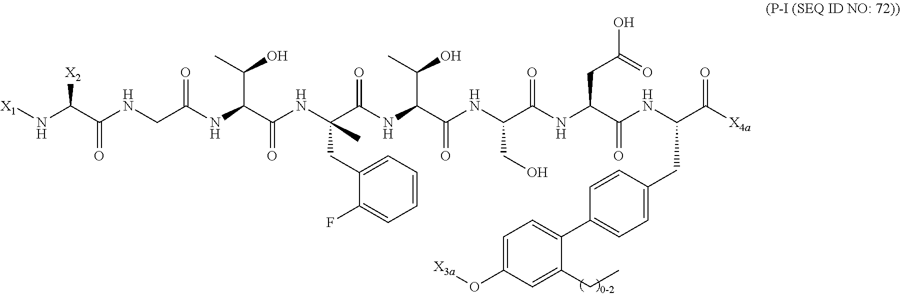
wherein:
X₁ is selected from H; 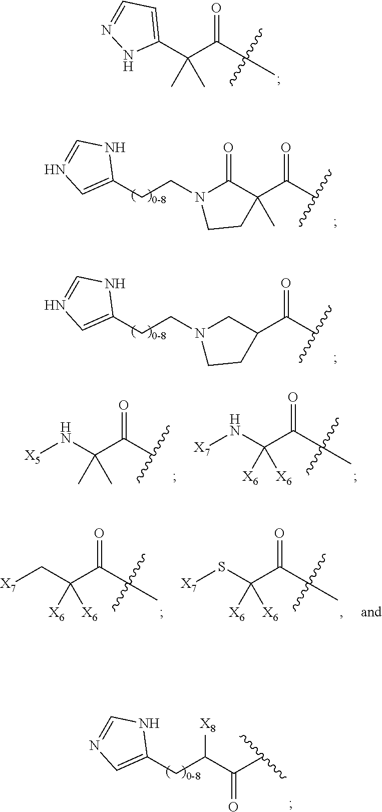
X₂ is selected from and 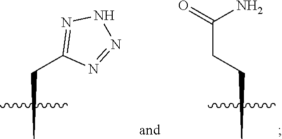
X₃ₐ is selected from —CH₃, —(CH₂)₁₋₄(OCH₂CH₂)₂₋₁₅—NH₂, —(CH₂)₂₋₆—NH₂, —(CH₂)₂₋₆—N₃, and —(CH₂)₂₋₆-Tr-(CH₂)₁₋₆—NH₂;
Tr is a triazole moiety;
X₄ₐ is —NH₂ or —OH;
X₅ is selected from —OH, —NH₂, —NH—OH, and 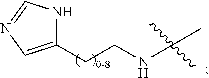
X₆ is independently at each occurrence selected from H, —OH, —CH₃, and —CH₂OH;
X₇ is selected from H, 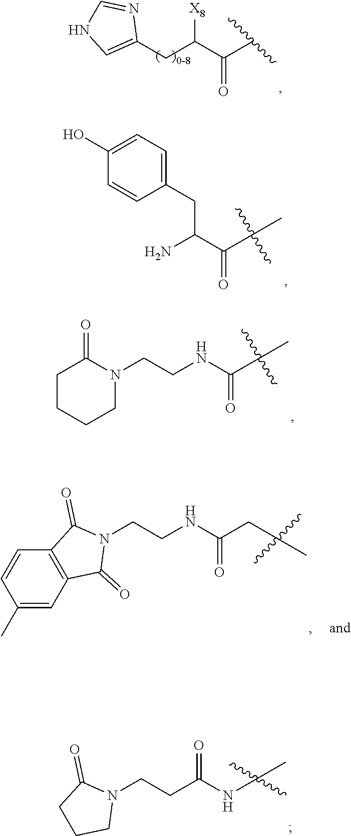
X₈ is selected from H, —OH, —NH₂, and 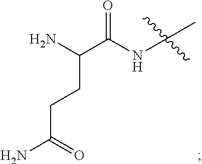 [SMILES: *NC(=O)C(N)CCC(N)=O]
Ar is selected from 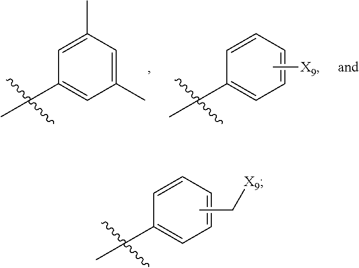
and
X₉ s selected from —NH₂, 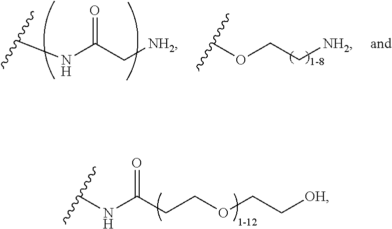
or a pharmaceutically acceptable salt thereof.

2. The compound of claim 1, wherein the compound of Formula (P-I) has a structure of Formula (P-Ia): 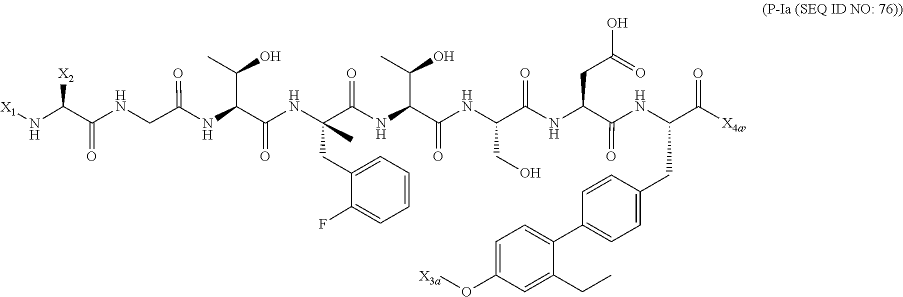
wherein
X₃ₐ is selected from —CH₃, —(CH₂)₂₋₆—NH₂, —(CH₂)₂₋₆—N₃, and —(CH₂)₂₋₆-Tr-(CH₂)₁₋₆—NH₂.

3. The compound of claim 1, wherein the compound of Formula (P-I) has a structure of Formula (P-Ib): 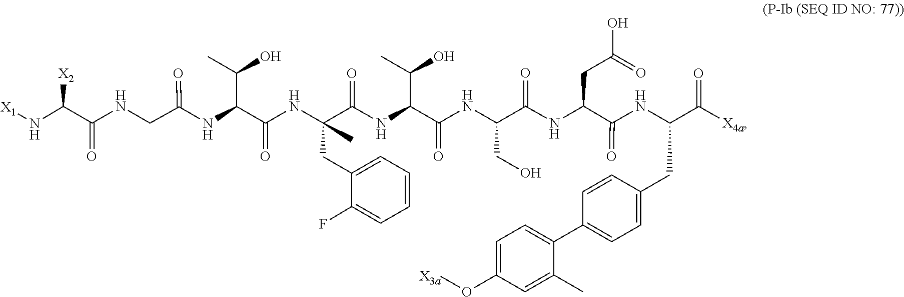
wherein
X₃ₐ is selected from —CH₃, —(CH₂)₂₋₆—NH₂, —(CH₂)₂₋₆—N₃, and —(CH₂)₂₋₆-Tr-(CH₂)₁₋₆—NH₂.

4. The compound of claim 1, wherein X₁ is

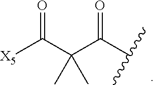

5. The compound of claim 1, wherein X₂ is

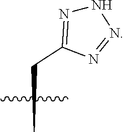

6. The compound of claim 1, wherein X₃ₐ is —(CH₂)₂₋₆—N₃.

7. The compound of claim 1, wherein X₅ is

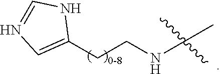

8. The compound of claim 1, wherein X₄ₐ is —NH₂.

9. (canceled)

10. The compound of claim 1, wherein the compound has the structure selected from the group consisting of:

| P# | Structure |
|---|---|
| P0 (SEQ ID NO: 146) | 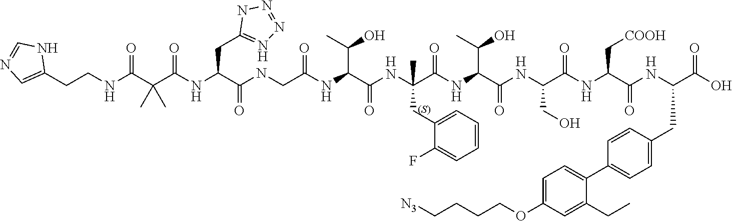 |
| P43 (SEQ ID NO: 188) | 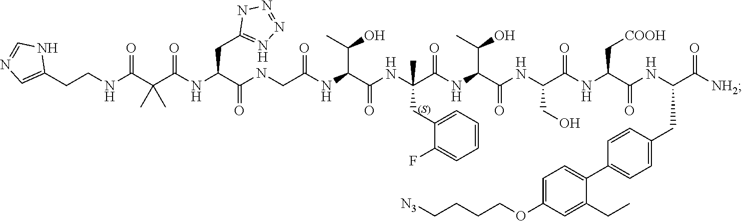 |
| P44 (SEQ ID NO: 189) | 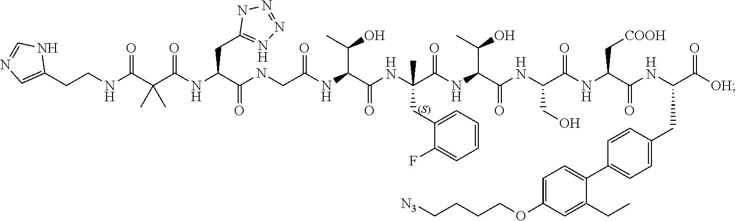 |
| P45 (SEQ ID NO: 190) | 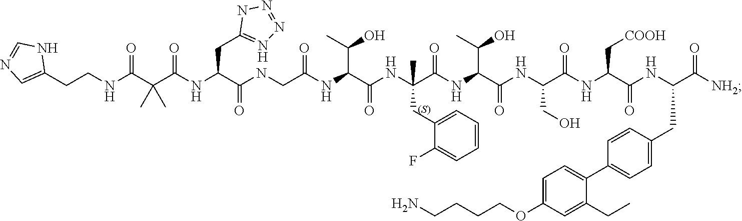 |
| P46 (SEQ ID NO: 191) | 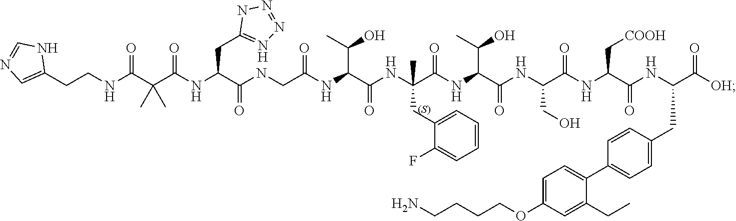 |
| P47 (SEQ ID NO: 192) | 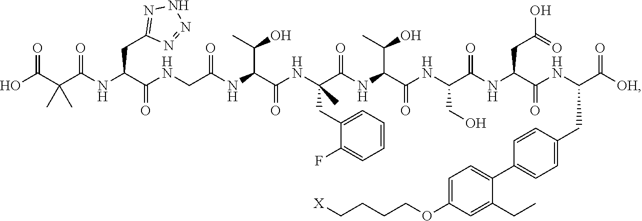 X = —NH₂ or —N₃; |
| P48 (SEQ ID NO: 193) | 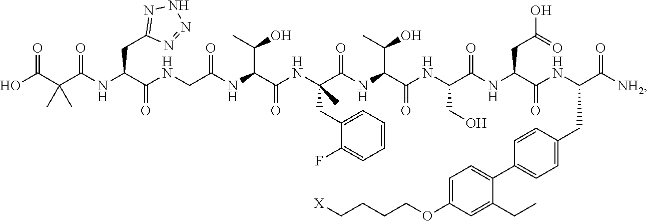 X = —NH₂ or —N₃; |
| P49 (SEQ ID NO: 194) | 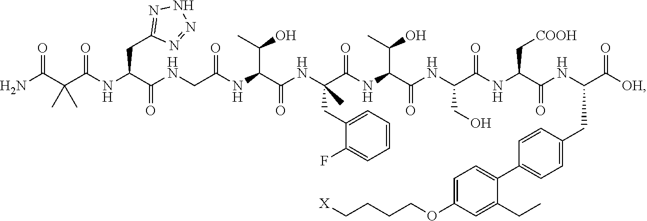 X = —NH₂ or —N₃; |
| P50 (SEQ ID NO: 195) | 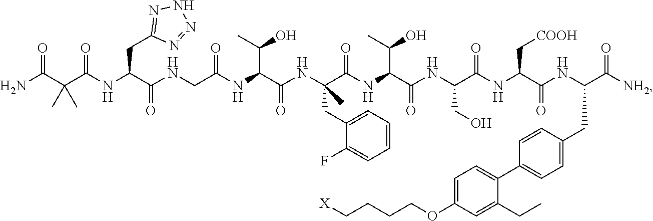 X = —NH₂ or —N₃; |
| P51 (SEQ ID NO: 196) | 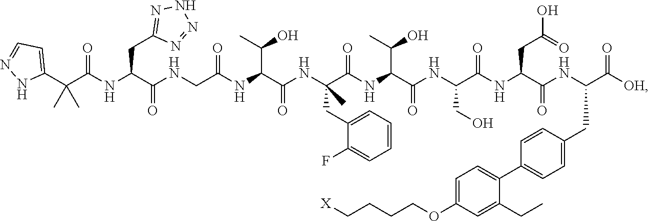 X = —NH₂ or —N₃; |
| P52 (SEQ ID NO: 197) | 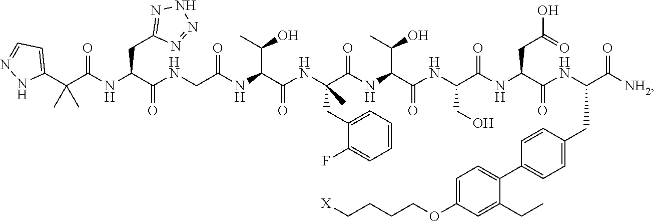 X = —NH₂ or —N₃; |
| P53 (SEQ ID NO: 198) | 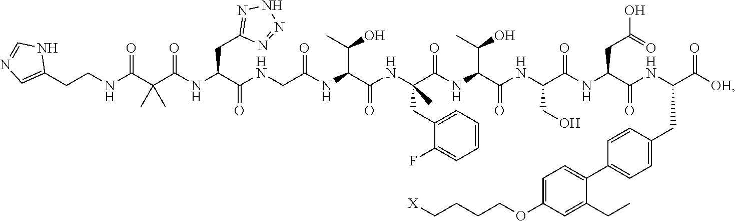 X = —NH₂ or —N₃; |
| P54 (SEQ ID NO: 199) | 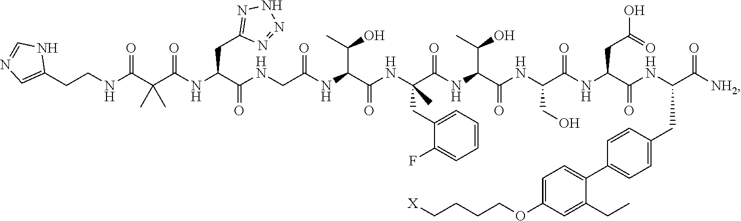 X = —NH₂ or —N₃; |
| P55 (SEQ ID NO: 200) | 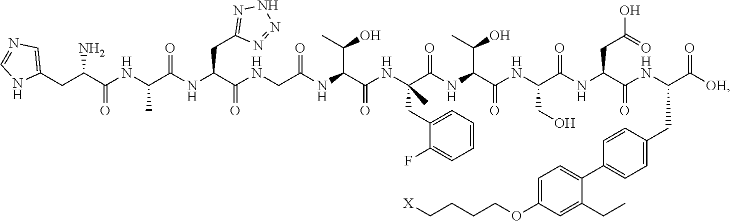 X = —NH₂ or —N₃; |
| P56 (SEQ ID NO: 201) | 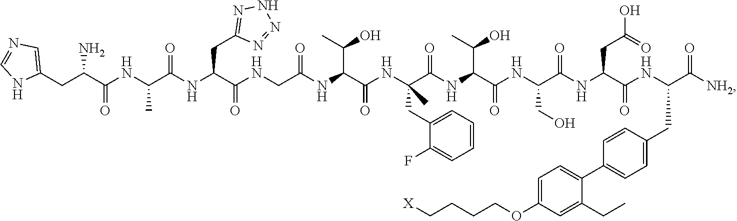 X = —NH₂ or —N₃; |
| P57 (SEQ ID NO: 202) | 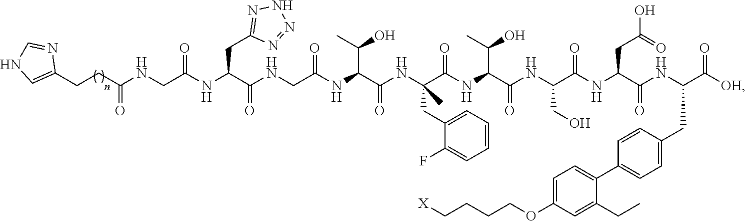 n is an integer of 1~7; X = —NH₂ or —N₃; |
| P58 (SEQ ID NO: 203) | 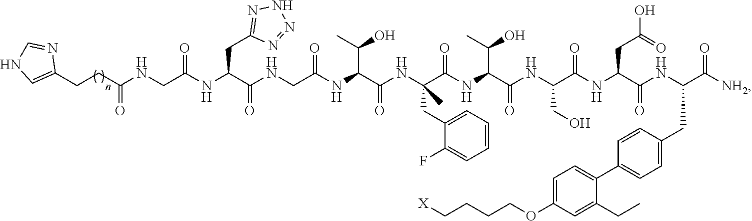 n is an integer of 1~7; X = —NH₂ or —N₃; |
| P59 (SEQ ID NO: 204) | 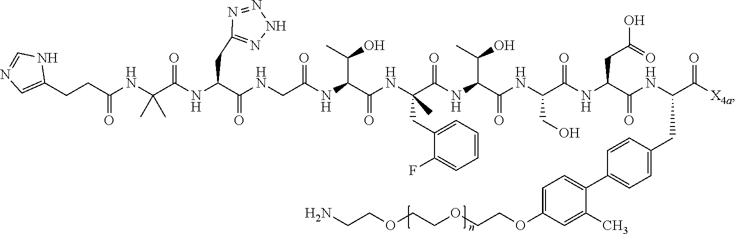 X₄a is —OH or —NH₂; n is an integer of one to twelve, and |
| P60 (SEQ ID NO: 205) | 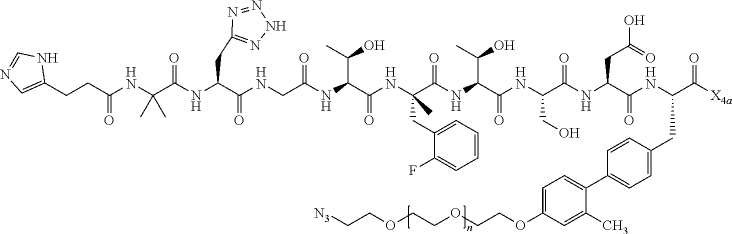 X₄a is —OH or —NH₂; n is an integer of one to twelve, |

or a pharmaceutically acceptable salt thereof.

11. A compound having a structure of Formula (LP-I): 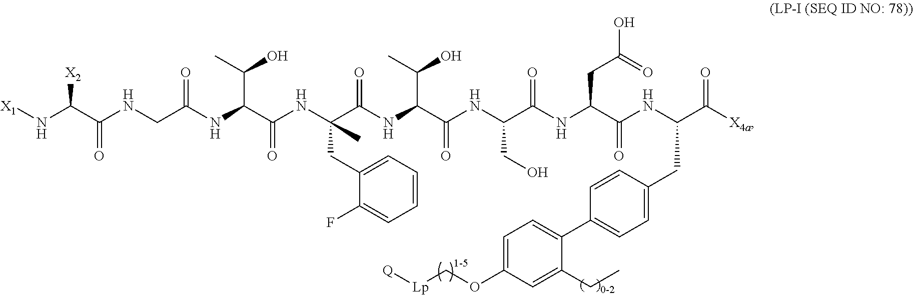
wherein:
X₁ is selected from H; 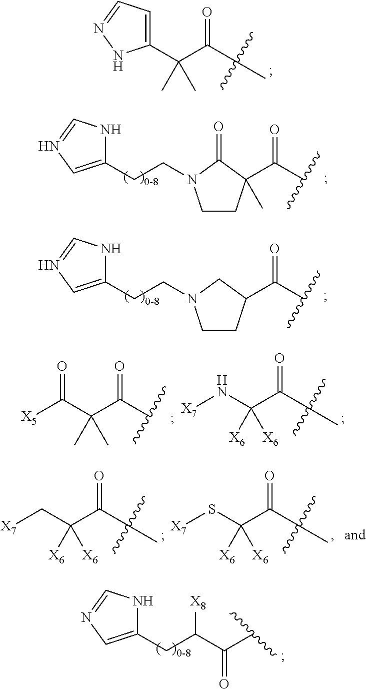
X₂ is selected from 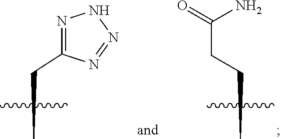
X₃ is selected from —CH₃, —(CH₂)₂₋₆—NH₂, —(CH₂)₂₋₆—N₃, and —(CH₂)₂₋₆-Tr-(CH₂)₁₋₆—NH₂, where
Tr is a triazole moiety;
Lₚ is absent or a linker comprising one or more of 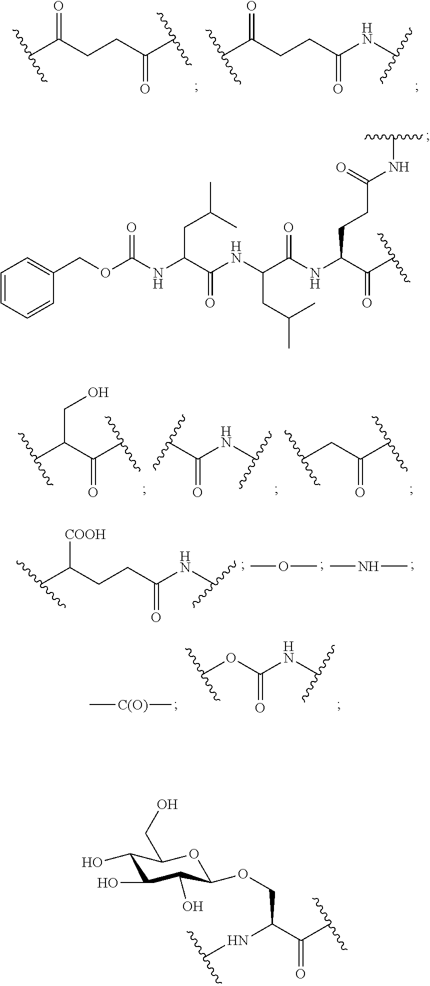
a cyclodextrin; —CH₂—O—; a polyethylene glycol (PEG) segment having 1 to 36-CH₂CH₂O-(EG) units; —(CH₂)₁₋₂₄-; a triazole; one or more amino acids selected from glycine, serine, glutamic acid, alanine, valine, threonine, leucine, and proline, and combinations thereof;
Q is a moiety selected from 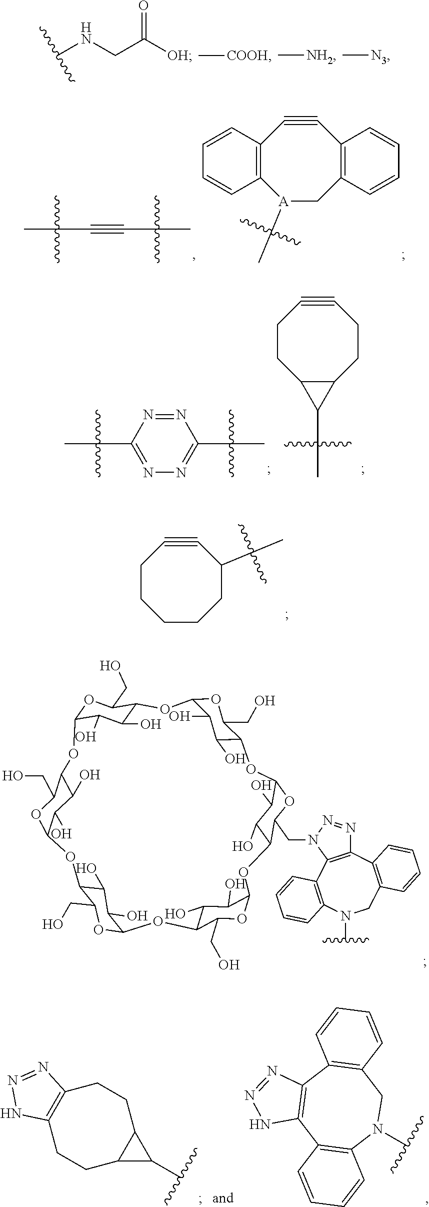
where A is C or N;
X₄ₐ is —NH₂ or —OH;
X₅ is selected from —OH, —NH₂, —NH—OH, and 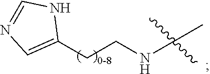
X₆ is independently at each occurrence selected from H, —OH, —CH₃, and —CH₂OH;
X₇ is selected from H, 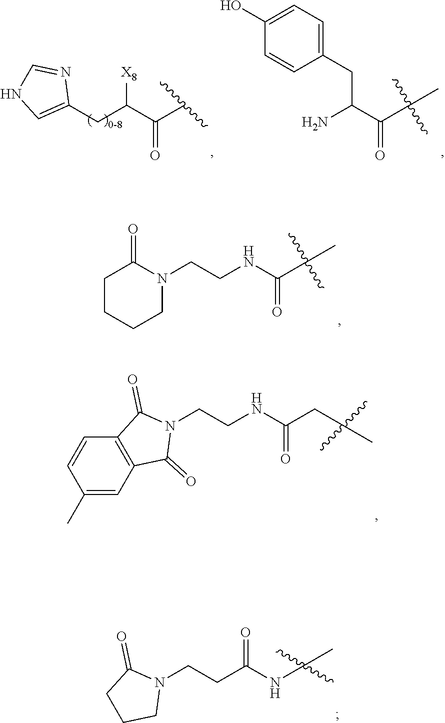
X₈ is selected from H, —OH, —NH₂, and 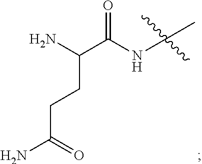 [SMILES: *NC(=O)C(N)CCC(N)=O]
Ar is selected from 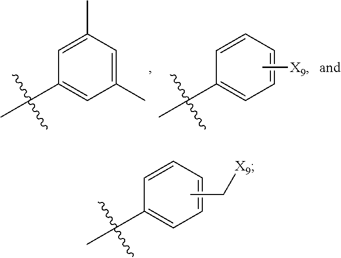
and
X₉ is selected from the group consisting of —NH₂, 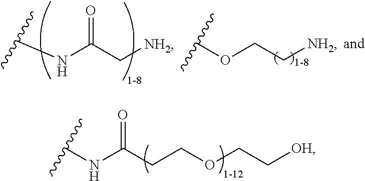
or a pharmaceutically acceptable salt thereof

12. The compound of claim 11, wherein the compound of Formula (LP-I) has a structure of Formula (LP-Ia) or Formula (LP-Ib):

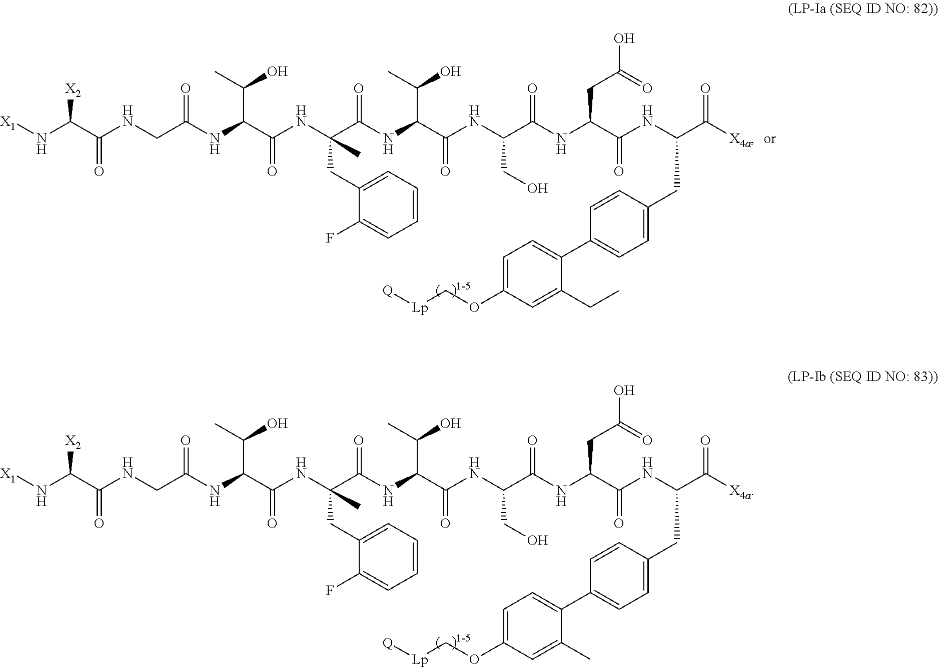

13. The compound of claim 11, wherein Lp comprises a polyethylene glycol (PEG) segment having 1 to 36-CH₂CH₂O-(EG) units.

14.-16. (canceled)

17. The compound of claim 11, wherein the Lp comprises one or more amino acids selected from glycine, serine, glutamic acid, alanine, valine, threonine, leucine, phenylalanine, citrulline, and proline, and combinations thereof.

18.-22. (canceled)

23. The compound of claim 11, wherein the Lp comprises a combination of a PEG segment having 1 to 36 EG units and a triazole group.

24. The compound of claim 11, wherein the Lp comprises a combination of a PEG segment having 1 to 36 EG units; a triazole group; and —CH₂—O— group.

25. The compound of claim 11, wherein the Lp comprises a combination of a PEG segment having 1 to 36 EG units; a triazole group; —CH₂—O— group; and —(CH₂)₁₋₂₄— group.

26. (canceled)

27. The compound of claim 11, wherein the Lp is

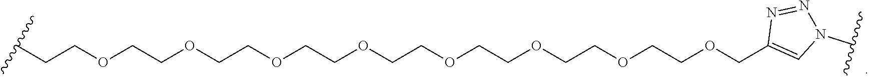

28. The compound of claim 11, wherein X₁ is

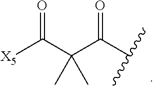

29. The compound of claim 11, wherein X₂ is

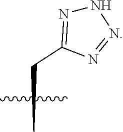

30. The compound of claim 11, wherein X₃ is —(CH₂)₂₋₆—N₃.

31. The compound of claim 11, wherein X₅ is

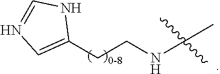

32. The compound of claim 11, wherein X₄ₐ is —NH₂ or -OH.

33. (canceled)

34. The compound of claim 11 having the structure selected from the group consisting of:

| LP# | Name | Structure |
|---|---|---|
| LP47 (SEQ ID NO: 144) | M6447 series | 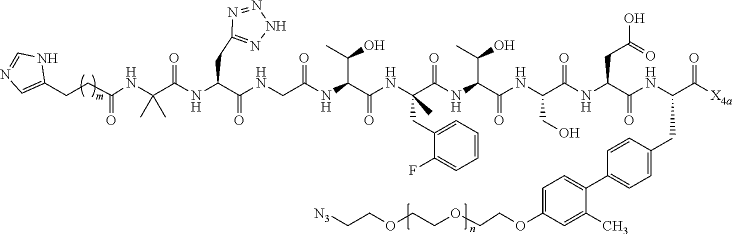 |
| | | X₄ₐ is —OH or —NH₂; m is an integer of one to seven; n is an integer of one to twelve, |
| LP48 (SEQ ID NO: 145) | M6447 series | 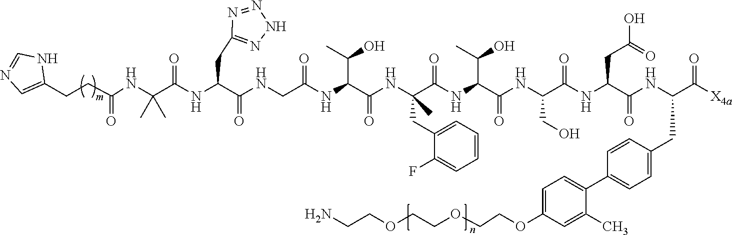 |
| | | X₄ₐ is —OH or —NH₂; m is an integer of one to seven; n is an integer of one to twelve, |

or a pharmaceutically acceptable salt thereof.

35. The compound of claim 11 having the structure selected from the group consisting of:

| LP′# | Structure |
|---|---|
| LP1′ (SEQ ID NO: 206) | 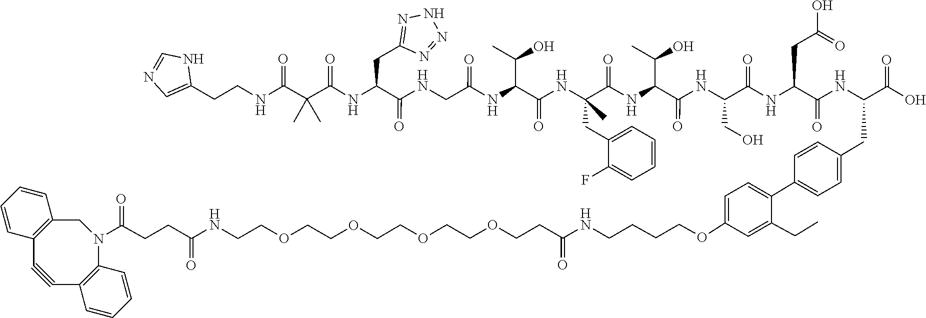 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP2′ (SEQ ID NO: 207) | 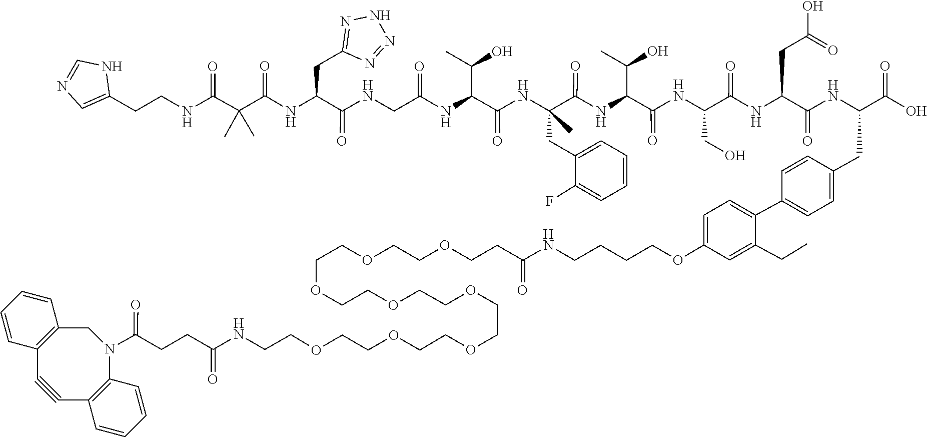 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP3′ (SEQ ID NO: 208) | 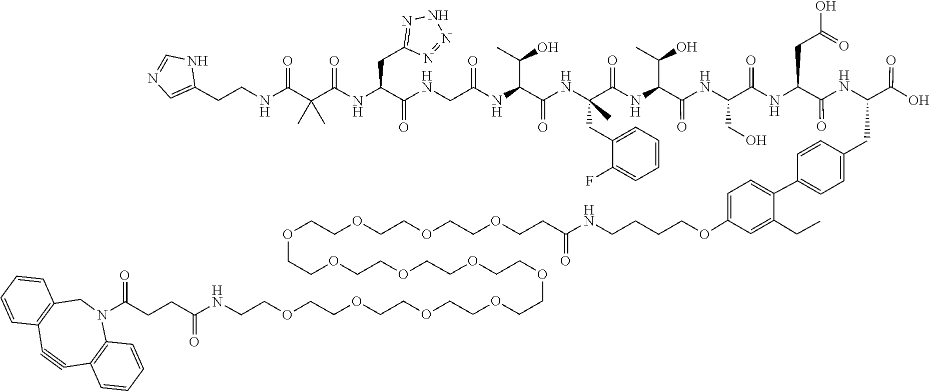 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP4′ (SEQ ID NO: 209) | 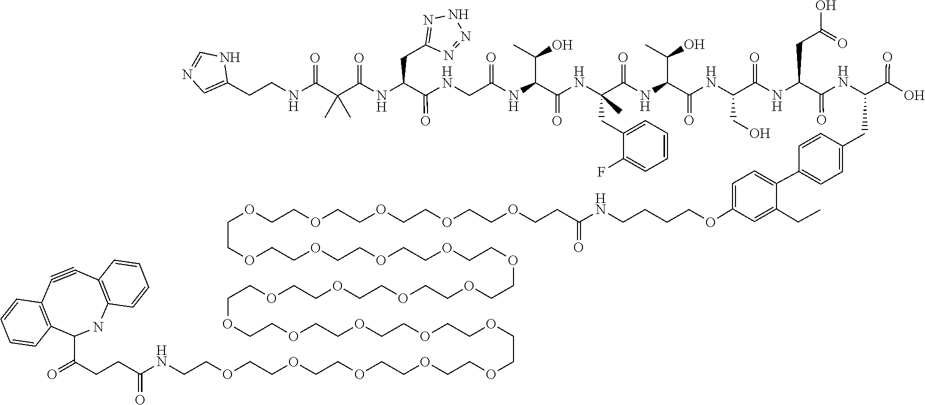 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)C2Nc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP5′ (SEQ ID NO: 210) | 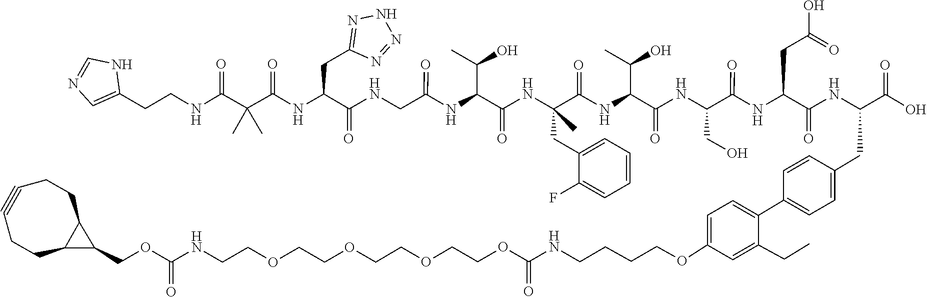 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP6′ (SEQ ID NOS 211 and 212, respec- tively) | 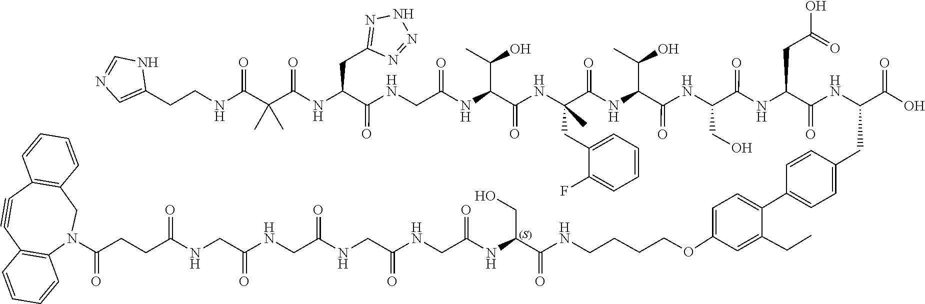 |
| LP7′ (SEQ ID NOS 213 and 214, respec- tively) | 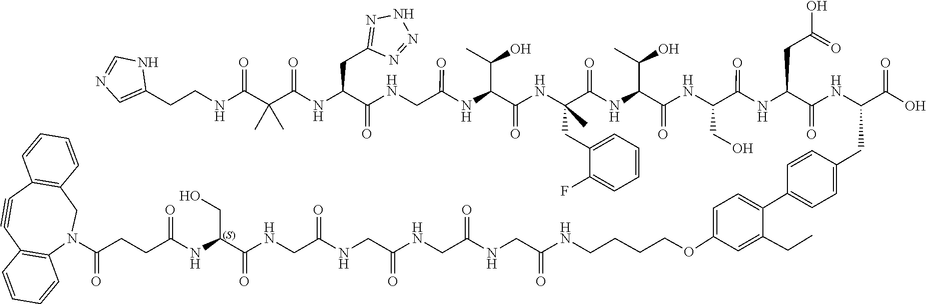 |
| LP8′ (SEQ ID NO: 215) | 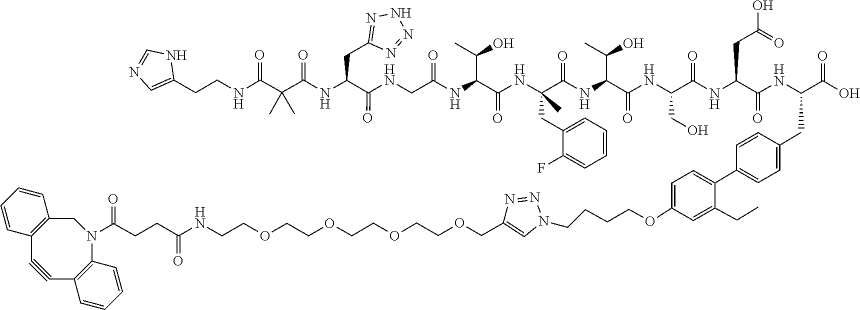 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP9′ (SEQ ID NO: 216) | 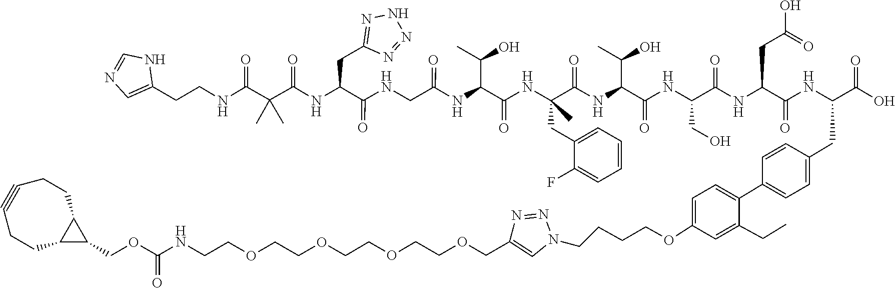 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP10′ (SEQ ID NO: 217) | 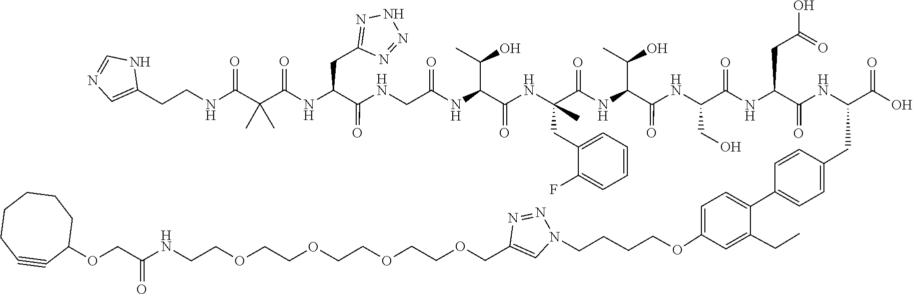 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP11′ (SEQ ID NO: 218) | 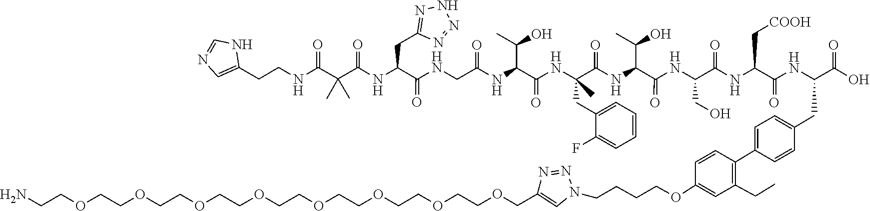 [SMILES: CCc1cc(OCCCCn2cc(COCCOCCOCCOCCOCCOCCOCCOCCN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP12′ (SEQ ID NO: 219) | 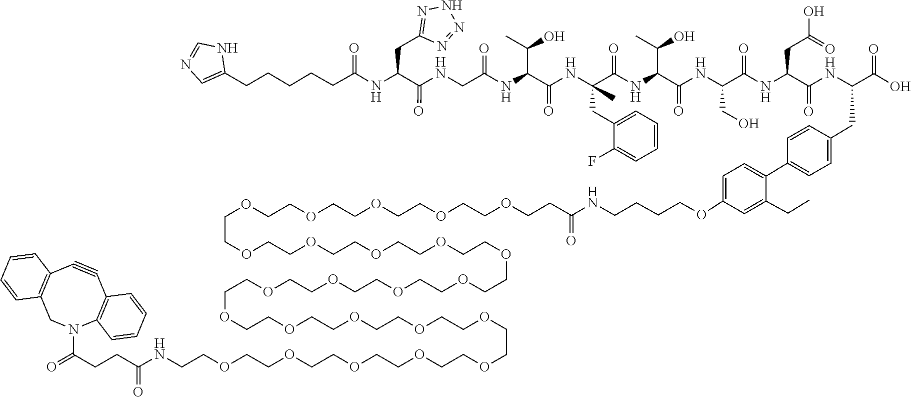 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(=O)O)cc1] |
| LP13′ (SEQ ID NO: 220) | 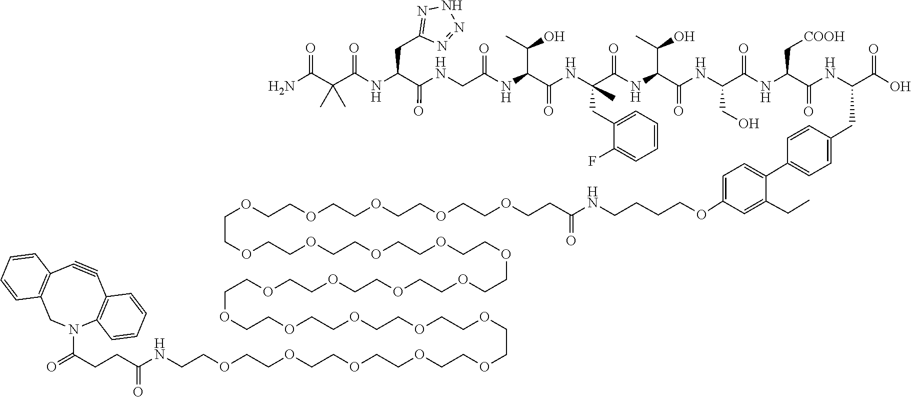 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)O)cc1] |
| LP14′ (SEQ ID NOS 221 and 222, respec- tively) | 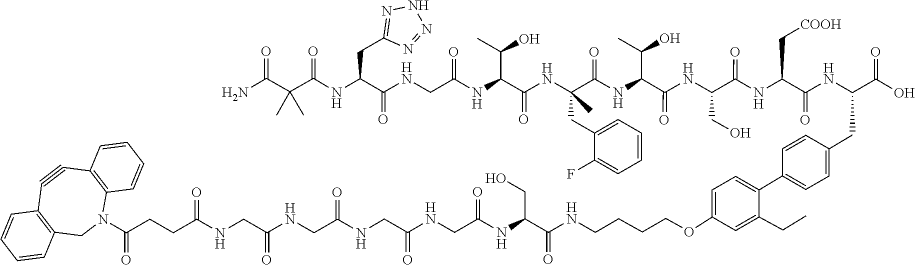 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(=O)O)cc1] |
| LP17′ (SEQ ID NOS 223 and 224, respec- tively) | 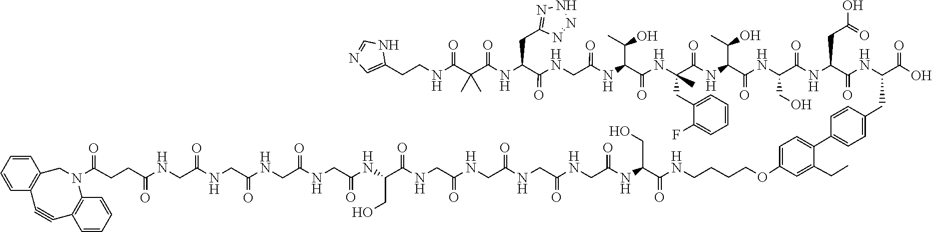 [SMILES: CCc1cc(OCCCCNC(=O)[C@H](CO)NC(=O)CNC(=O)CNC(=O)CNC(=O)CNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP18′ (SEQ ID NOS 225 and 226, respec- tively) | 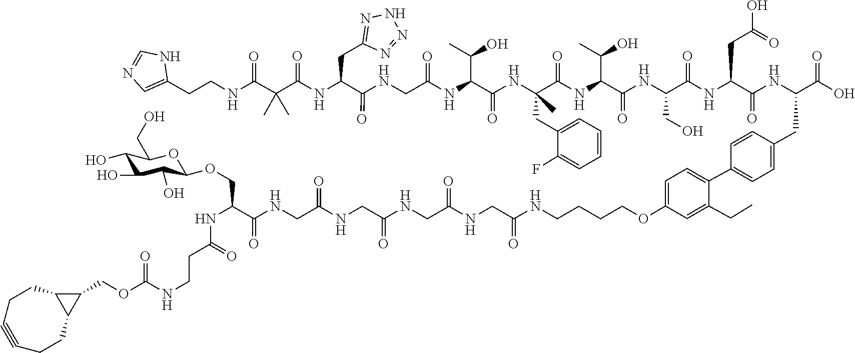 |
| LP19′ (SEQ ID NOS 227 and 228, respec- tively) | 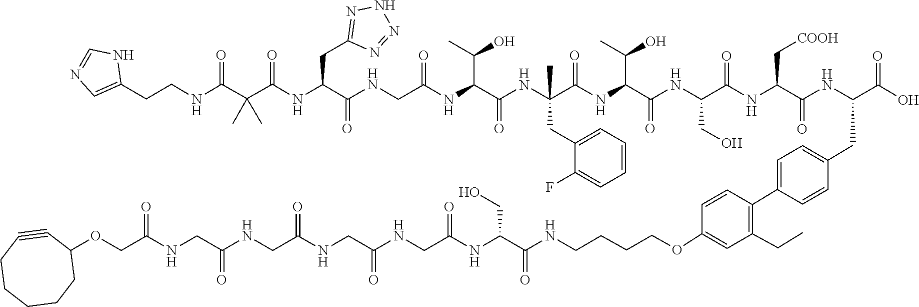 |
| LP20′ (SEQ ID NOS 229 and 230, respec- tively) | 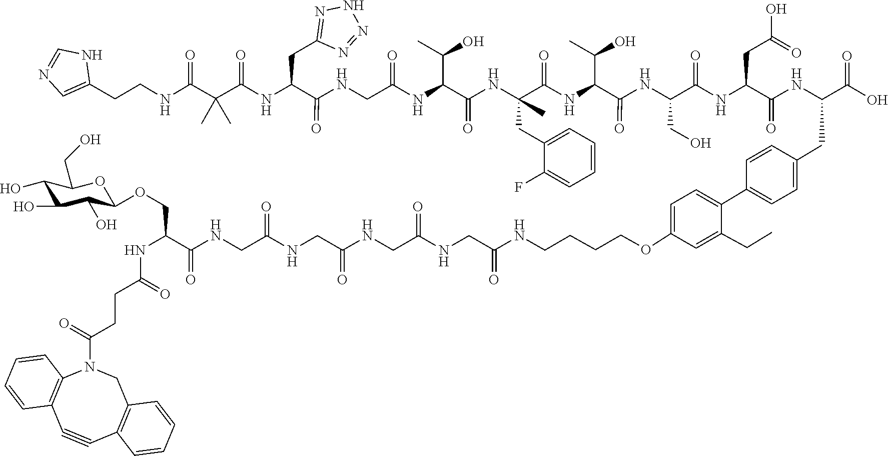 |
| LP22′ (SEQ ID NO: 231) | 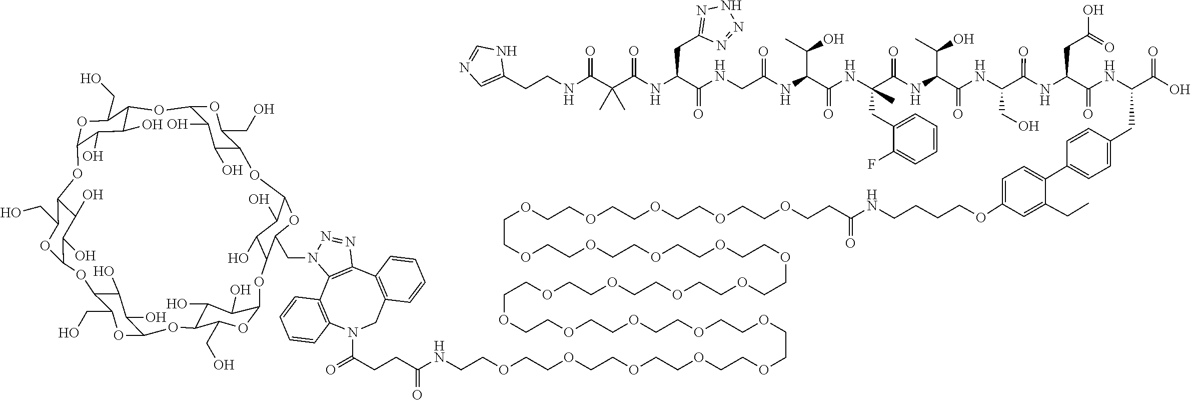 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP24′ (SEQ ID NO: 232) | 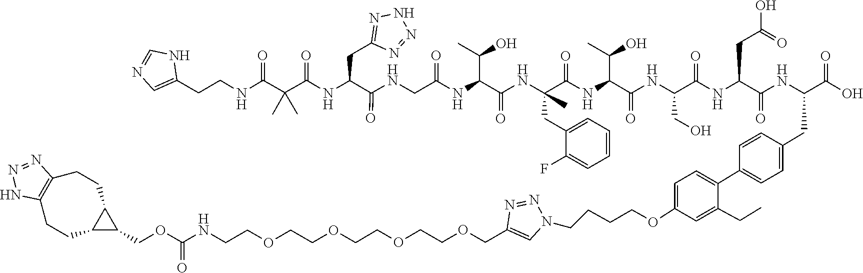 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP25′ (SEQ ID NO: 233) | 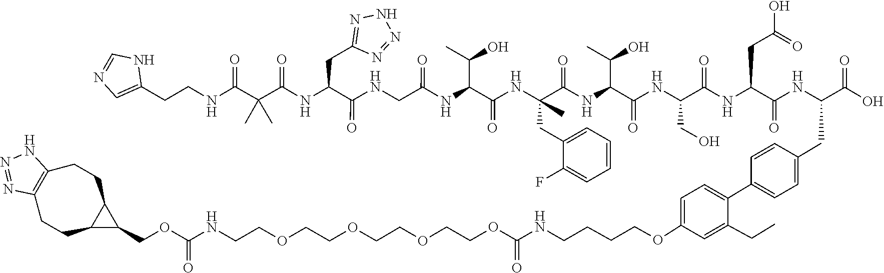 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(=O)O)cc1] |
| LP26′ (SEQ ID NOS 234 and 235, respec- tively) | 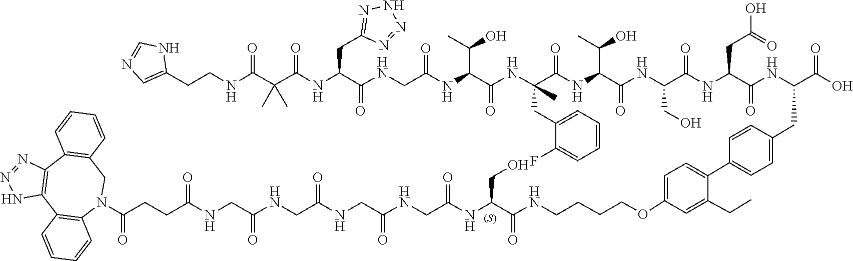 |
| LP27′ (SEQ ID NO: 236) | 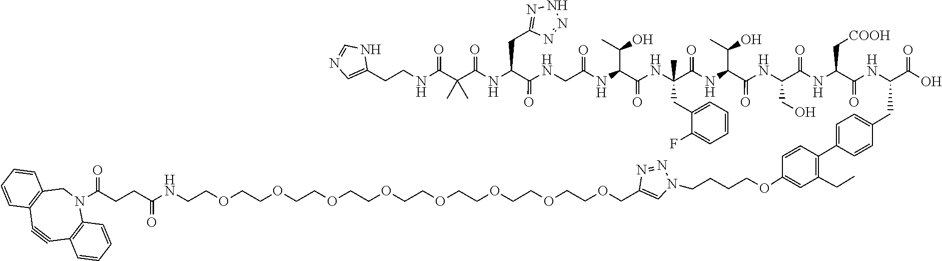 |
| LP28′ (SEQ ID NO: 237) | 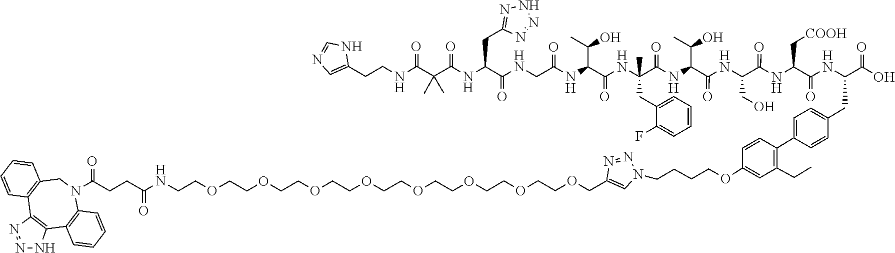 |
| LP29′ (SEQ ID NO: 238) | 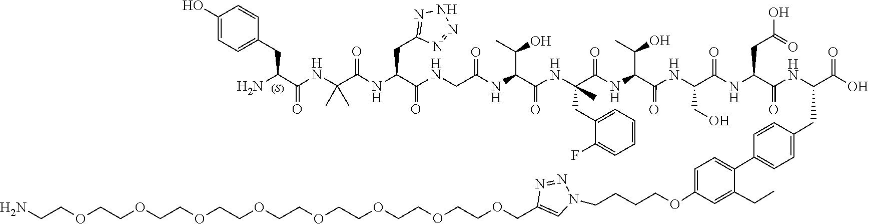 |
| LP30′ (SEQ ID NO: 239) | 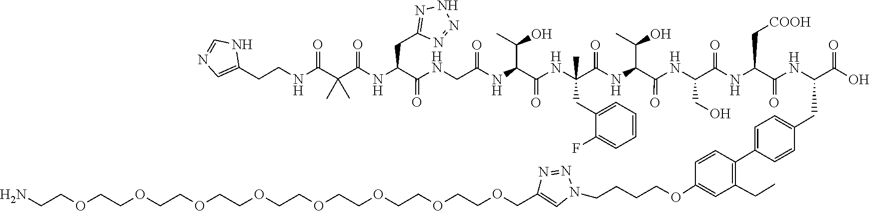 |
| LP31′ (SEQ ID NO: 240) | 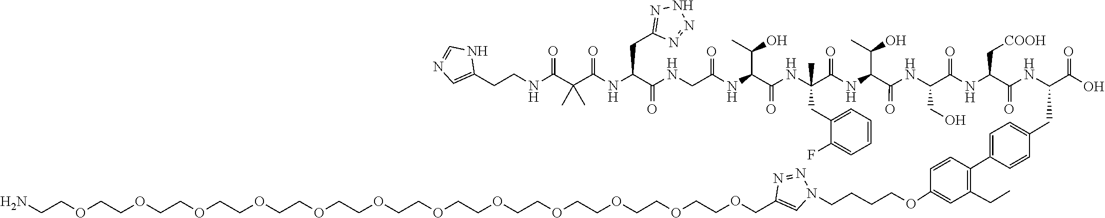 |
| LP33′ (SEQ ID NO: 241) | 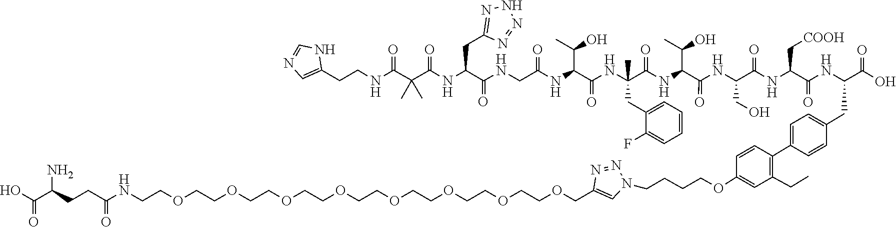 |
| LP34′ (SEQ ID NOS 242 and 243, respec- tively) | 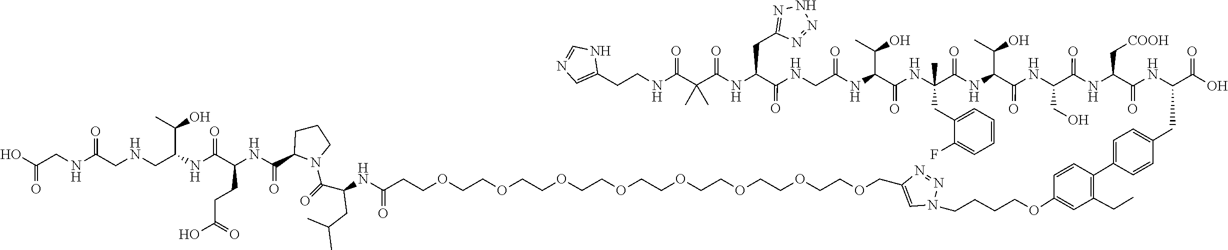 |
| LP35′ (SEQ ID NOS 244 and 245, respec- tively) | 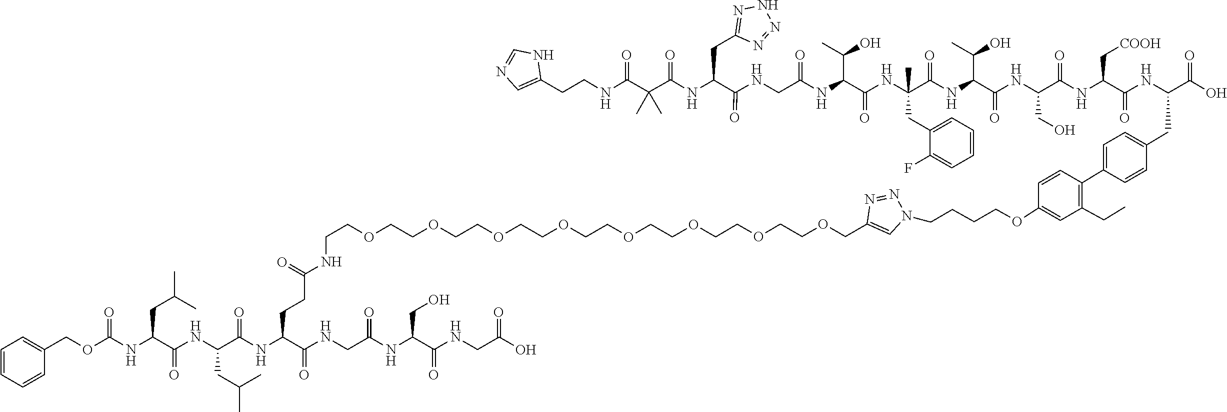 |
| LP36′ (SEQ ID NOS 246 and 352, respec- tively) | 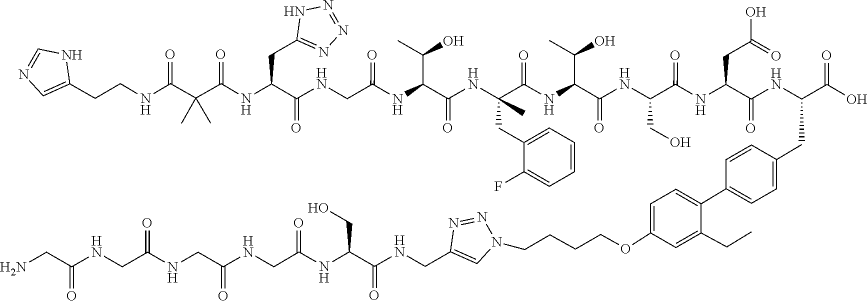 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP37′ (SEQ ID NOS 247 and 248, respec- tively) | 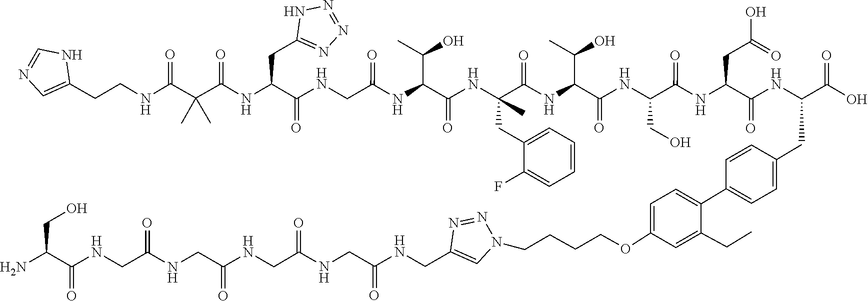 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP38′ (SEQ ID NOS 249 and 67, respec- tively) | 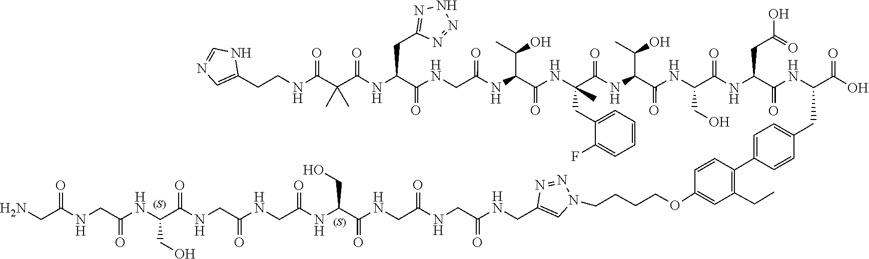 |
| LP39′ (SEQ ID NOS 250 and 251, respec- tively) | 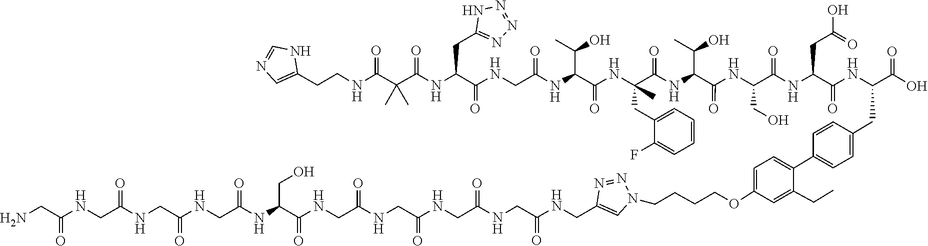 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP40′ (SEQ ID NOS 252 and 253, respec- tively) | 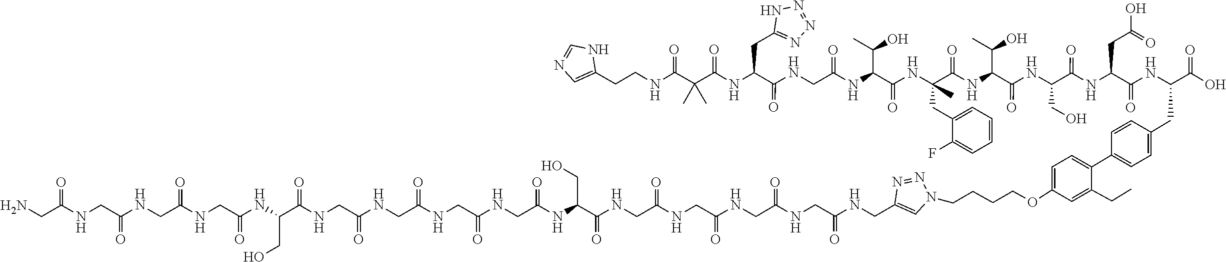 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(=O)O)cc1] |
| LP41′ (SEQ ID NOS 254 and 255, respec- tively) | 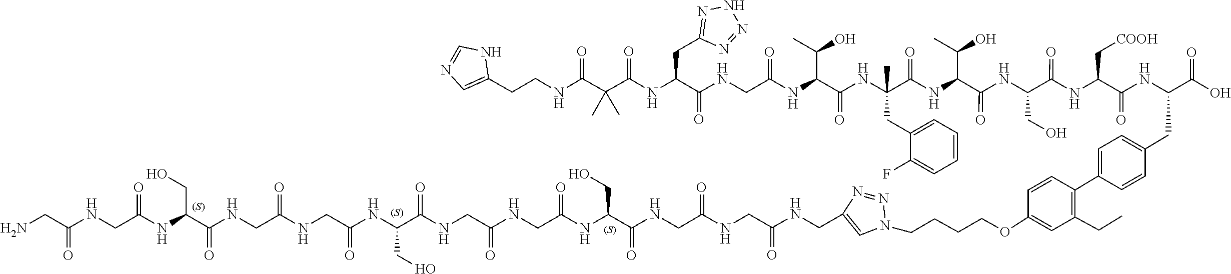 |
| LP42′ (SEQ ID NOS 256 and 257, respec- tively) | 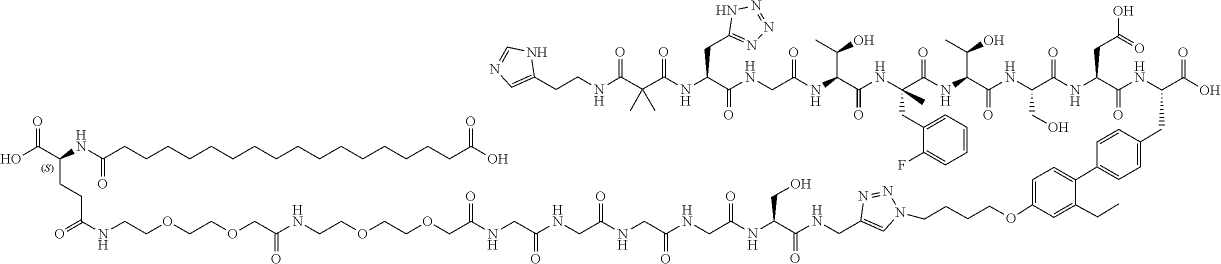 |
| LP43′ (SEQ ID NOS 258 and 259, respec- tively) | 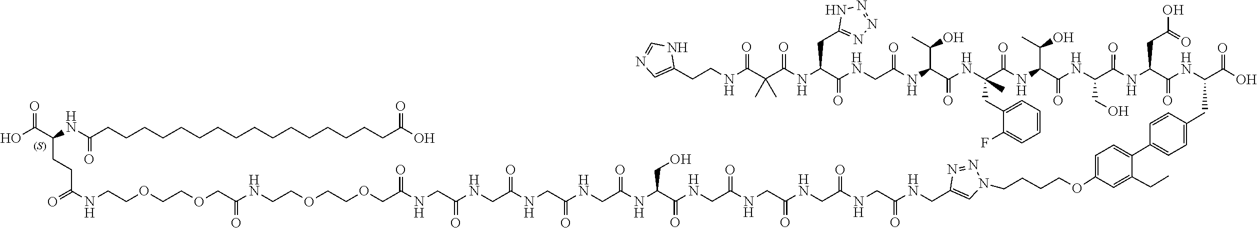 |
| LP44′ (SEQ ID NO: 260) | 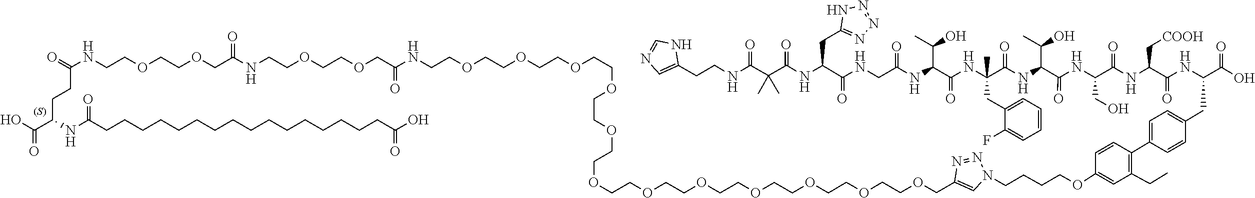 |
| LP45′ (SEQ ID NO: 261) | 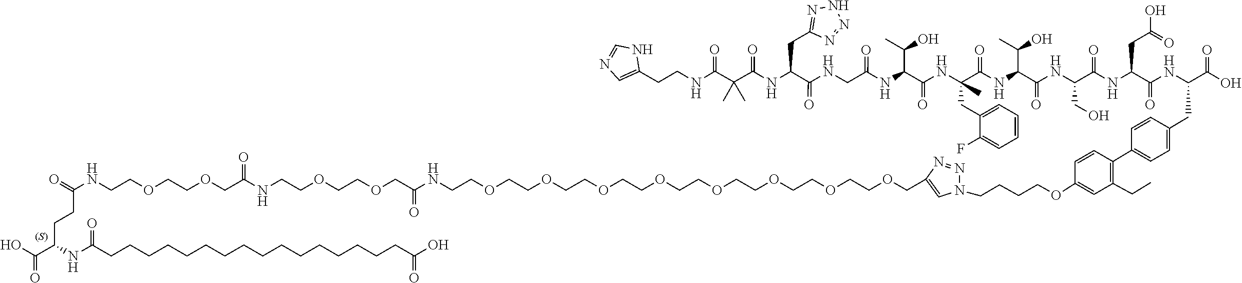 |
| LP46′ (SEQ ID NO: 262) | 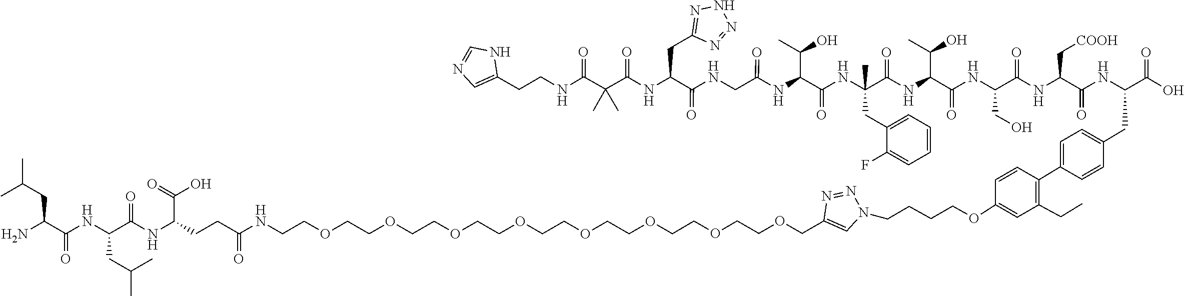 |
| LP1′a (SEQ ID NO: 263) | 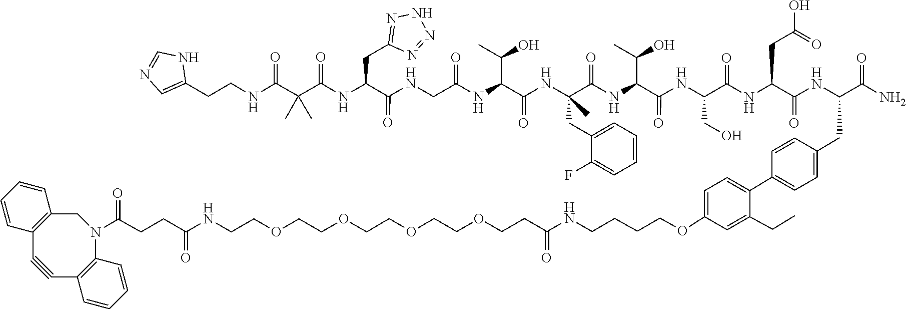 [SMILES: CCc1cc(OCCCCNC(=O)CCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP2′a (SEQ ID NO: 264) | 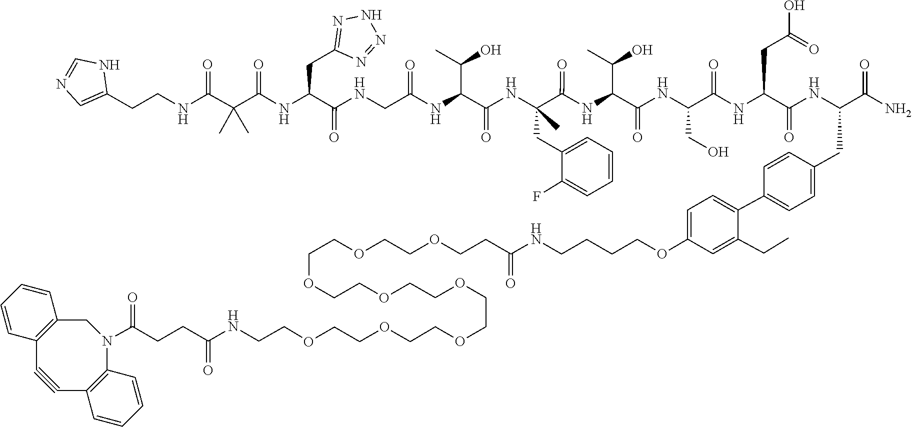 [SMILES: CCc1cc(OCCCCNC(=O)CCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP3′a (SEQ ID NO: 265) | 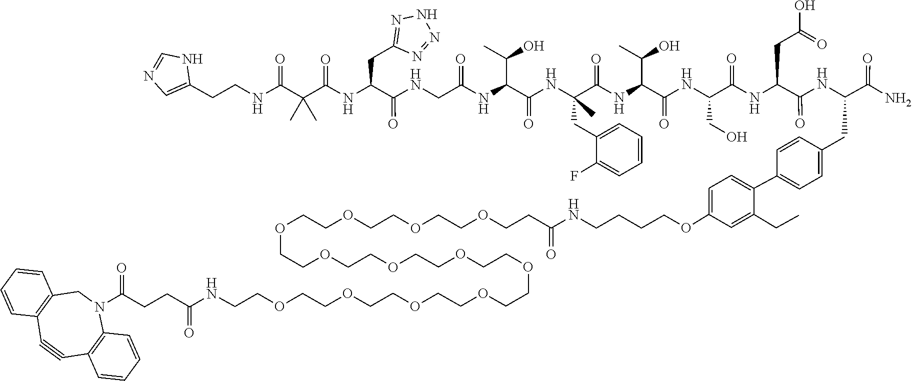 [SMILES: CCc1cc(OCCCCNC(=O)CCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP4′a (SEQ ID NO: 266) | 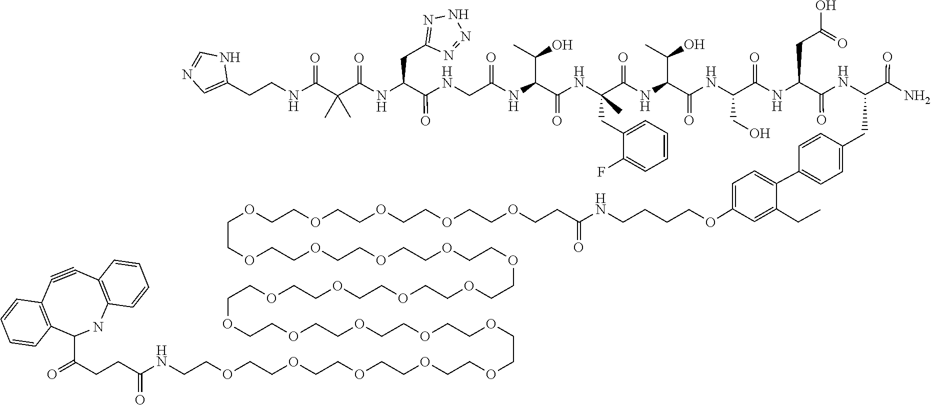 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)C2Nc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP5′a (SEQ ID NO: 267) | 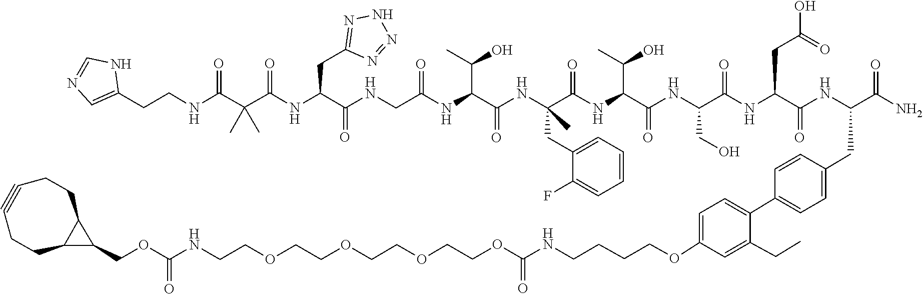 [SMILES: CCc1cc(OCCCCNC(=O)OCCOCCOCCOCCNC(=O)OC[C@@H]2[C@@H]3CCC#CCC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP6′a (SEQ ID NOS 268 and 269, respec- tively) | 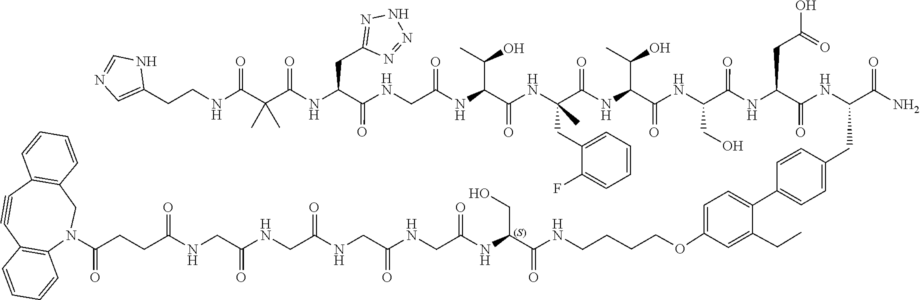 |
| LP7′a (SEQ ID NOS 270 and 271, respec- tively) | 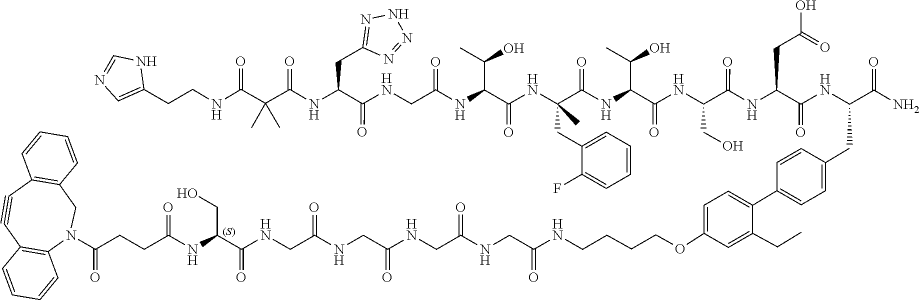 |
| LP8′a (SEQ ID NO: 272) | 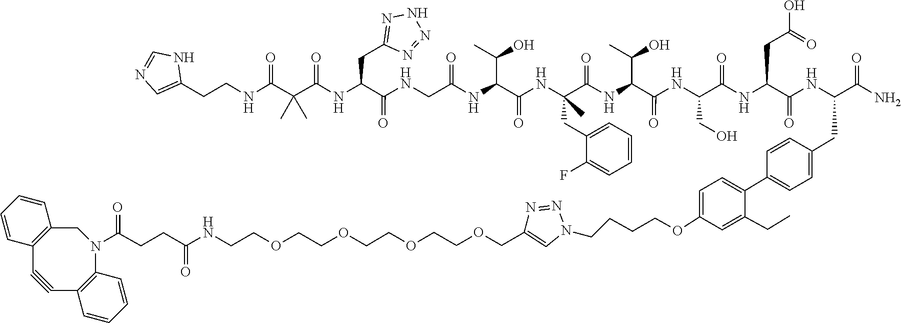 [SMILES: CCc1cc(OCCCCn2cc(COCCOCCOCCOCCNC(=O)CCC(=O)N3Cc4ccccc4C#Cc4ccccc43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP9′a (SEQ ID NO: 273) | 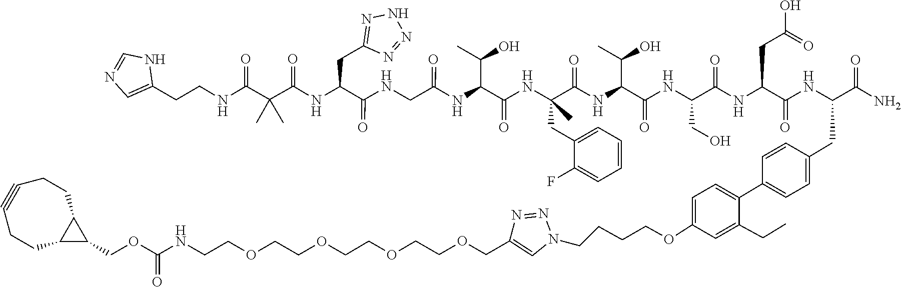 [SMILES: CCc1cc(OCCCCn2cc(COCCOCCOCCOCCNC(=O)OC[C@@H]3[C@@H]4CCC#CCC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP10′a (SEQ ID NO: 274) | 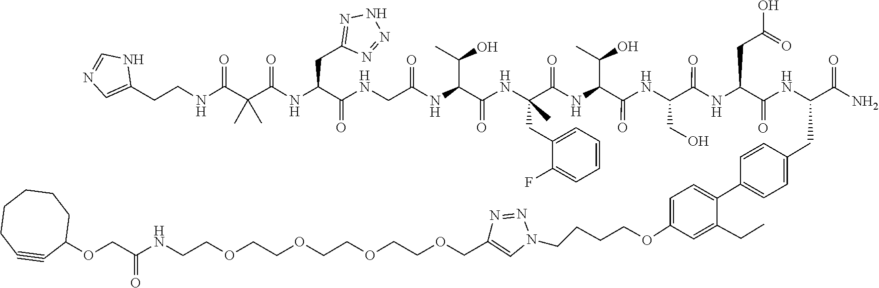 [SMILES: CCc1cc(OCCCCn2cc(COCCOCCOCCOCCNC(=O)COC3C#CCCCCC3)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP11′a (SEQ ID NO: 275) | 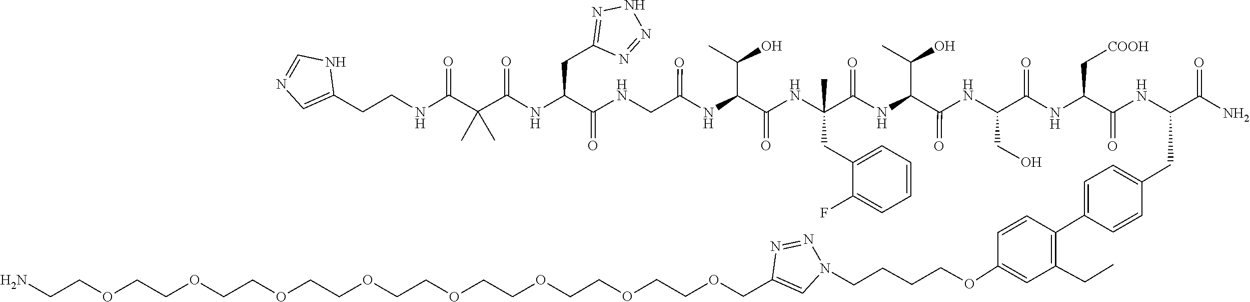 |
| LP12′a (SEQ ID NO: 276) | 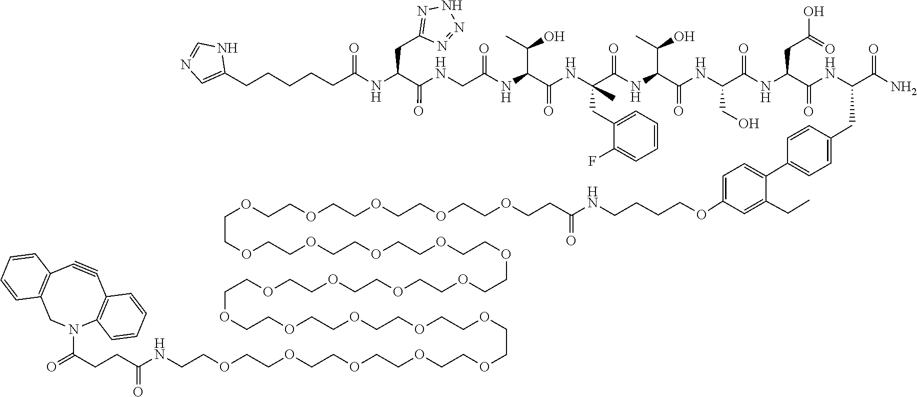 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)CCCCCc2cnc[nH]2)[C@@H](C)O)[C@@H](C)O)C(N)=O)cc1] |
| LP13′a (SEQ ID NO: 277) | 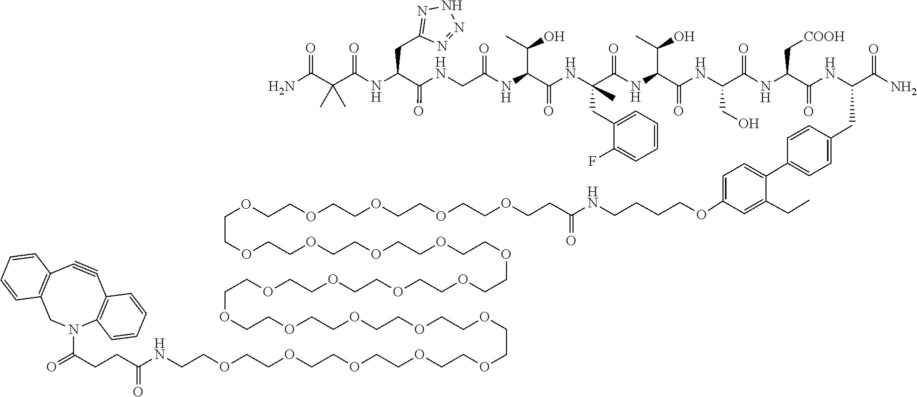 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(N)=O)cc1] |
| LP14′a (SEQ ID NOS 278 and 279, respec- tively) | 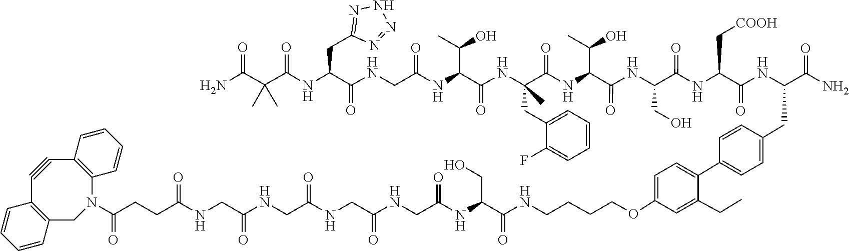 [SMILES: CCc1cc(OCCCCNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(N)=O)[C@@H](C)O)[C@@H](C)O)C(N)=O)cc1] |
| LP17′a (SEQ ID NOS 280 and 281, respec- tively) | 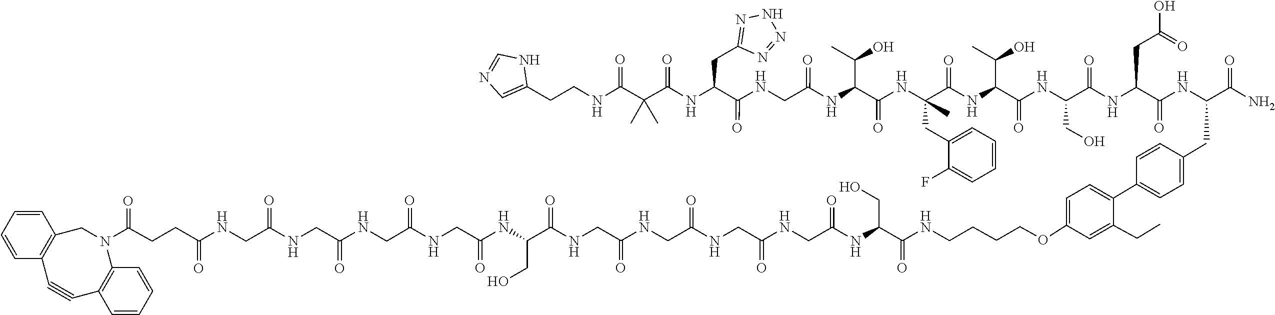 [SMILES: CCc1cc(OCCCCNC(=O)[C@H](CO)NC(=O)CNC(=O)CNC(=O)CNC(=O)CNC(=O)[C@H](CO)NC(=O)CNC(=O)CNC(=O)CNC(=O)CNC(=O)CCC(=O)N2Cc3ccccc3C#Cc3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP18′a (SEQ ID NOS 282 and 283, respec- tively) | 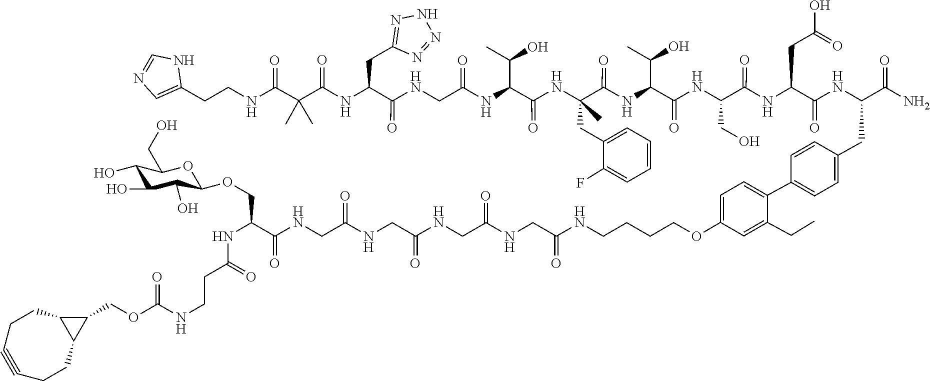 |
| LP19′a (SEQ ID NOS 284 and 285, respec- tively) | 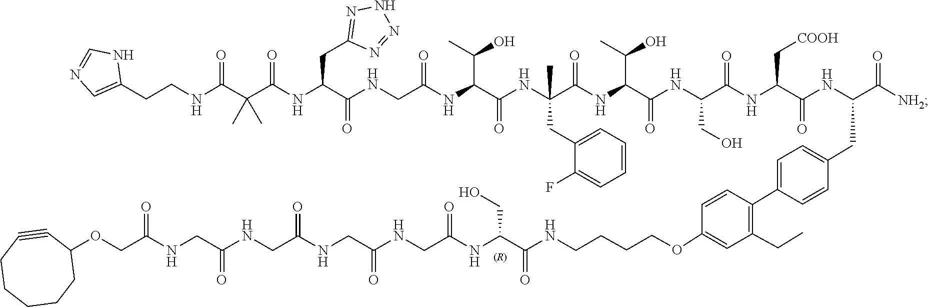 |
| LP20′a (SEQ ID NOS 286 and 287, respec- tively) | 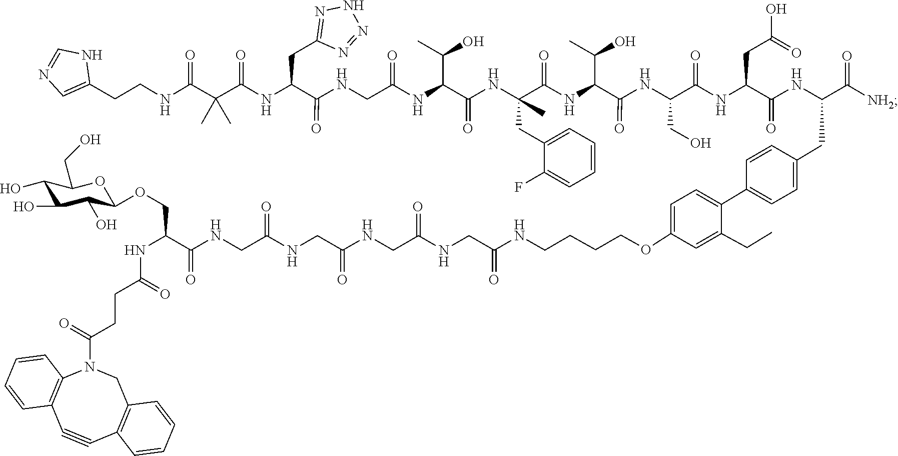 |
| LP22′a (SEQ ID NO: 288) | 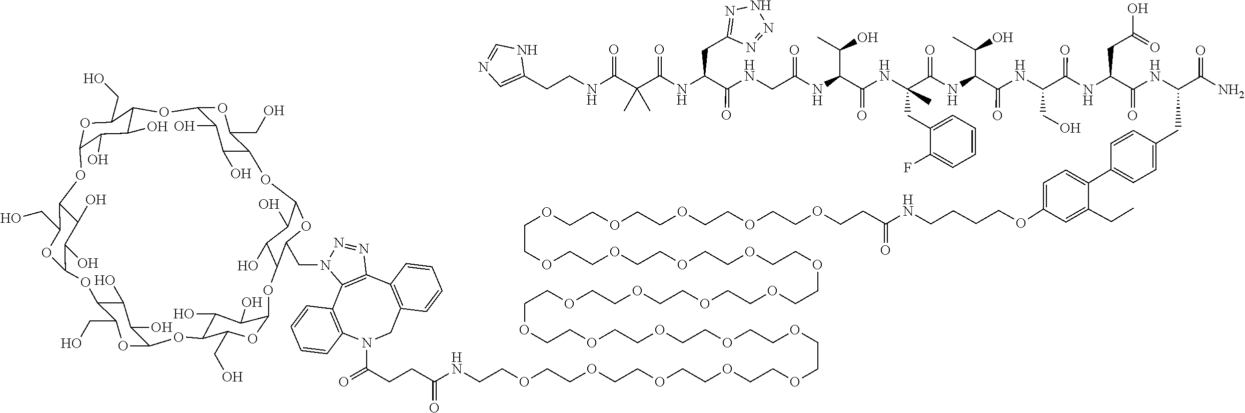 [SMILES: CCc1cc(OCCCCNC(=O)CCOCCOCCOCCOCCOCCOCCOCCOCCOCCOCCOCCOCCOCCOCCOCCOCCOCCOCCOCCOCCOCCOCCOCCOCCNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP24′a (SEQ ID NO: 289) | 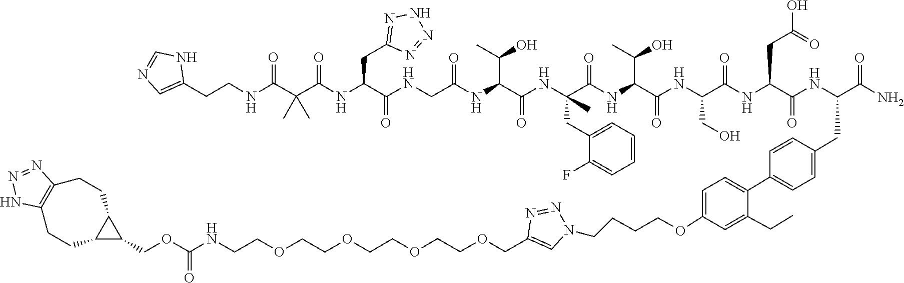 [SMILES: CCc1cc(OCCCCn2cc(COCCOCCOCCOCCNC(=O)OC[C@@H]3[C@@H]4CCc5[nH]nnc5CC[C@@H]43)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP25′a (SEQ ID NO: 290) | 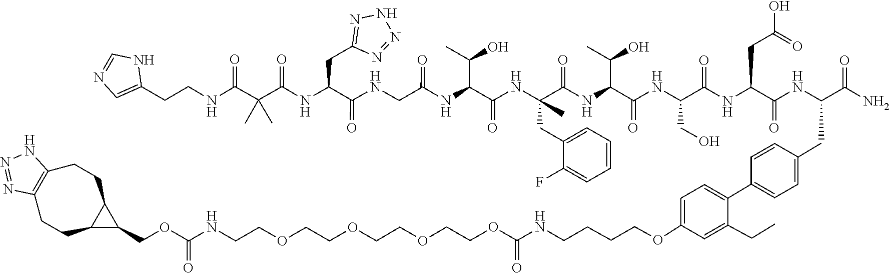 [SMILES: CCc1cc(OCCCCNC(=O)OCCOCCOCCOCCNC(=O)OC[C@@H]2[C@@H]3CCc4[nH]nnc4CC[C@@H]32)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nn[nH]n2)NC(=O)C(C)(C)C(=O)NCCc2cnc[nH]2)[C@@H](C)O)[C@@H](C)O)C(N)=O)cc1] |
| LP26′a (SEQ ID NOS 291 and 292, respec- tively) | 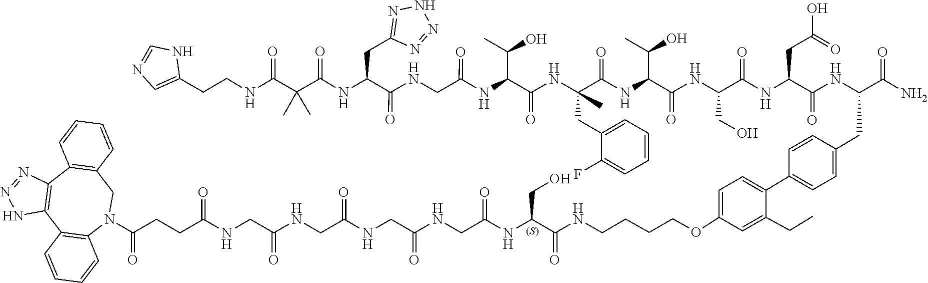 |
| LP27′a (SEQ ID NO: 293) | 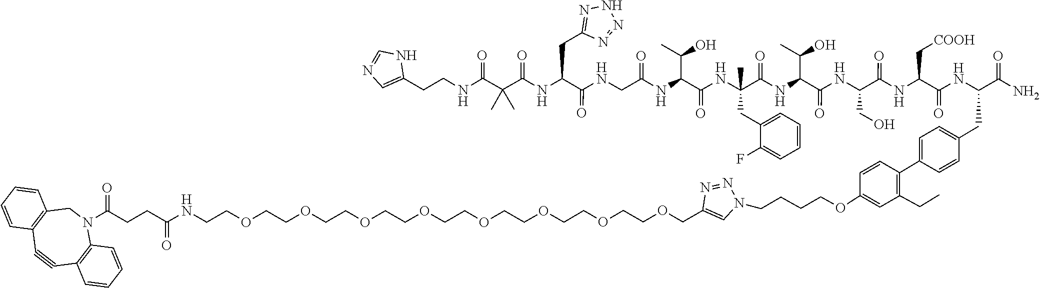 |
| LP28′a (SEQ ID NO: 294) | 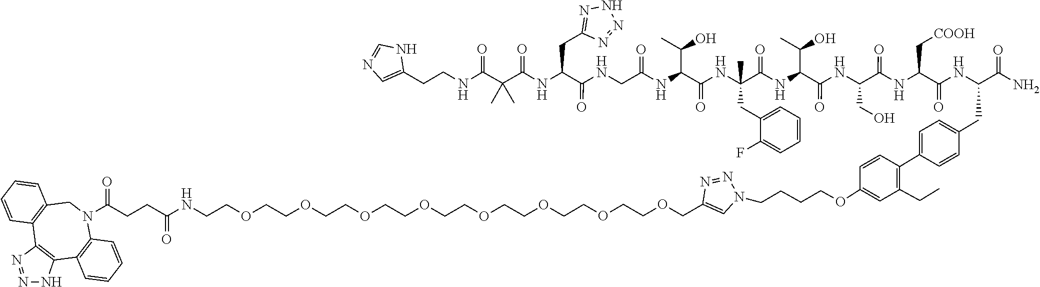 |
| LP29′a (SEQ ID NO: 295) | 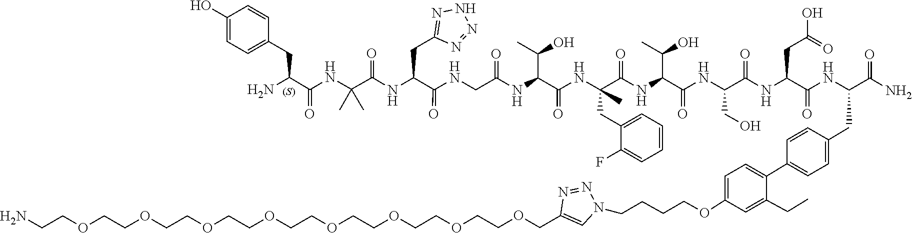 |
| LP30′a (SEQ ID NO: 296) | 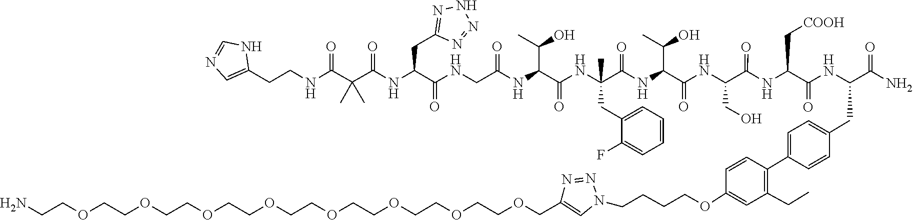 |
| LP31′a (SEQ ID NO: 297) | 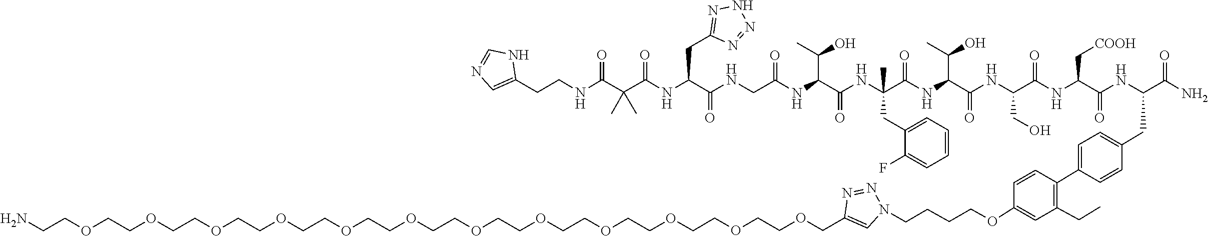 |
| LP33′a (SEQ ID NO: 298) | 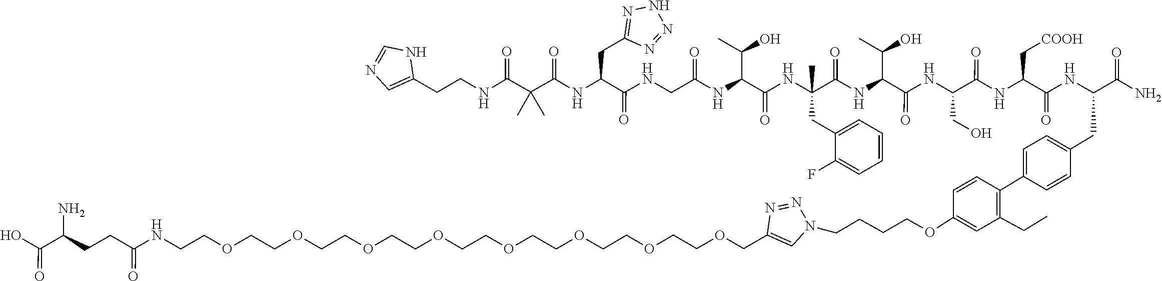 |
| LP34′a (SEQ ID NOS 299 and 300, respec- tively) | 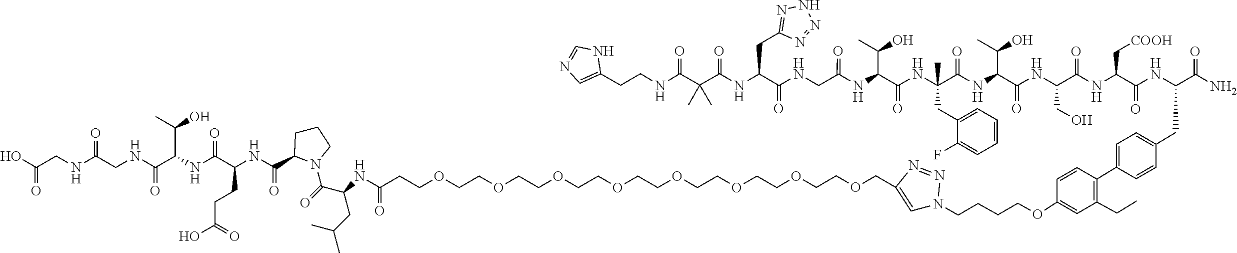 |
| LP35′a (SEQ ID NOS 301 and 302, respec- tively) | 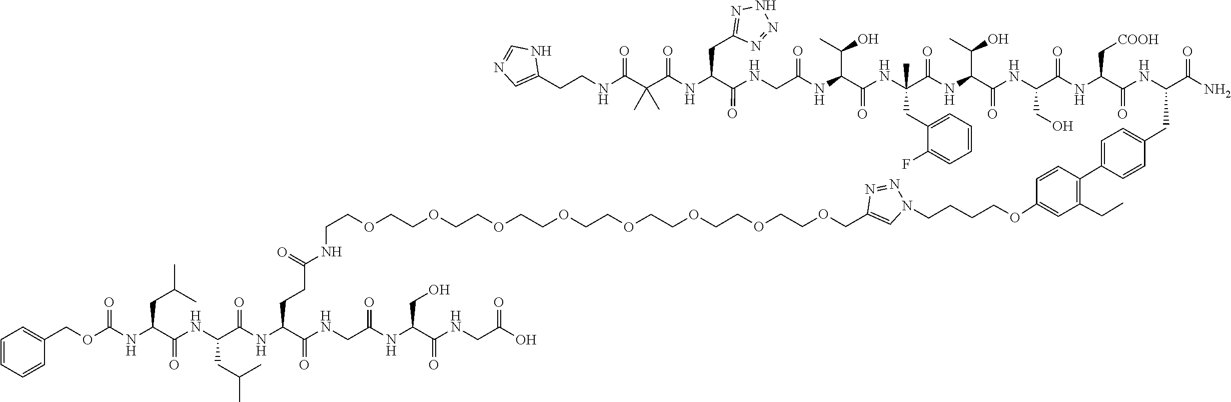 |
| LP36′a (SEQ ID NOS 303 and 304, respec- tively) | 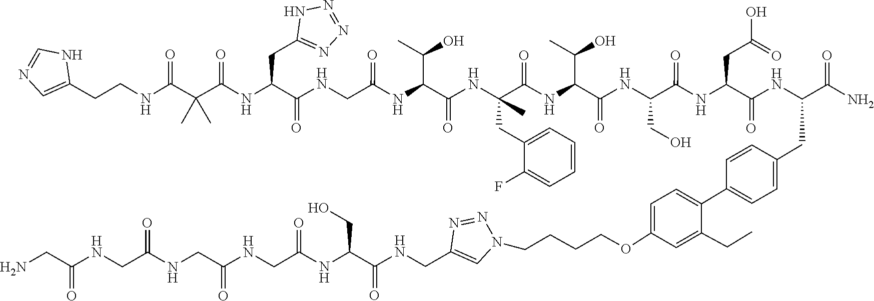 [SMILES: CCc1cc(OCCCCn2cc(CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP37′a (SEQ ID NOS 305 and 306, respec- tively) | 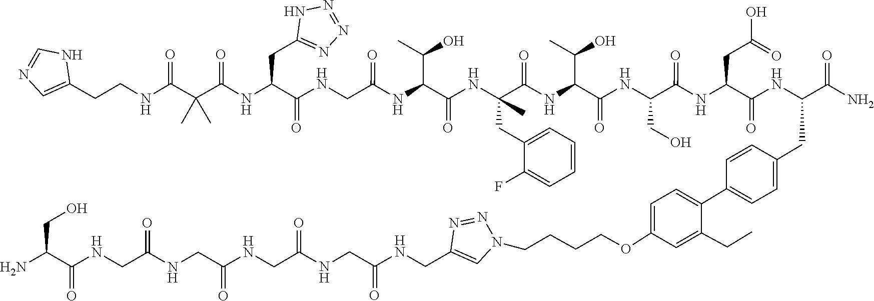 [SMILES: CCc1cc(OCCCCn2cc(CNC(=O)CNC(=O)CNC(=O)CNC(=O)CNC(=O)[C@@H](N)CO)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP38′a (SEQ ID NOS 307 and 353, respec- tively) | 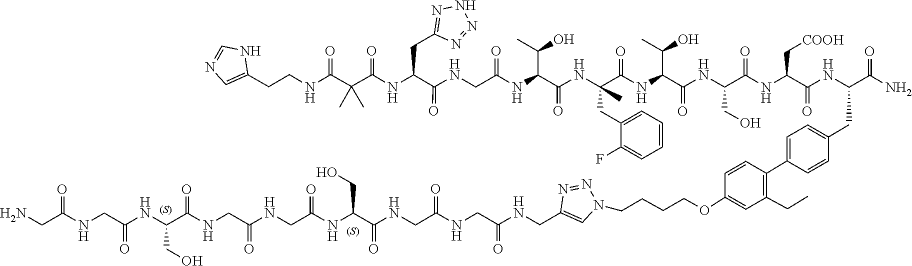 |
| LP39′a (SEQ ID NOS 308 and 309, respec- tively) | 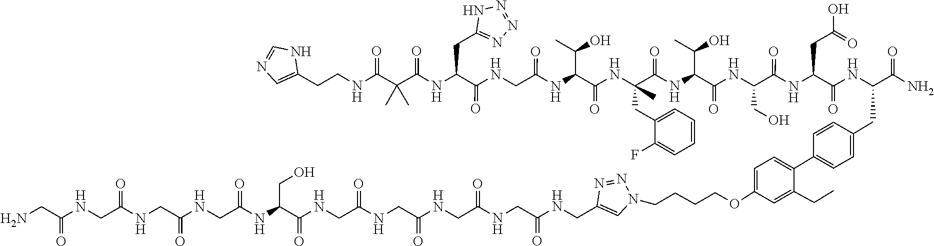 [SMILES: CCc1cc(OCCCCn2cc(CNC(=O)CNC(=O)CNC(=O)CNC(=O)CNC(=O)[C@H](CO)NC(=O)CNC(=O)CNC(=O)CNC(=O)CN)nn2)ccc1-c1ccc(C[C@H](NC(=O)[C@H](CC(=O)O)NC(=O)[C@H](CO)NC(=O)[C@@H](NC(=O)[C@](C)(Cc2ccccc2F)NC(=O)[C@@H](NC(=O)CNC(=O)[C@H](Cc2nnn[nH]2)NC(=O)C(C)(C)C(=O)NCCc2cnc[nH]2)[C@@H](C)O)[C@@H](C)O)C(N)=O)cc1] |
| LP40′a (SEQ ID NOS 310 and 311, respec- tively) | 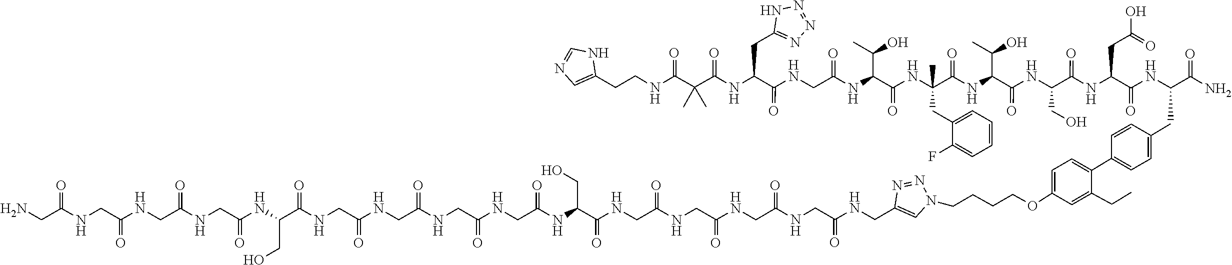 |
| LP41′a (SEQ ID NOS 312 and 313, respec- tively) | 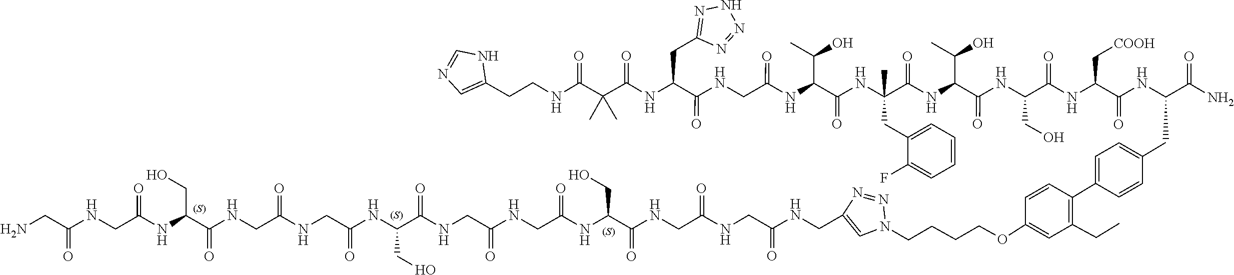 |
| LP42′a (SEQ ID NOS 314 and 315, respec- tively) | 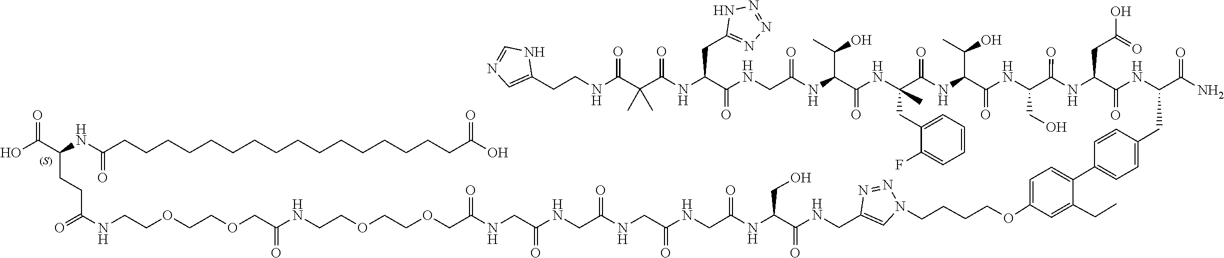 |
| LP43′a (SEQ ID NOS 316 and 317, respec- tively) | 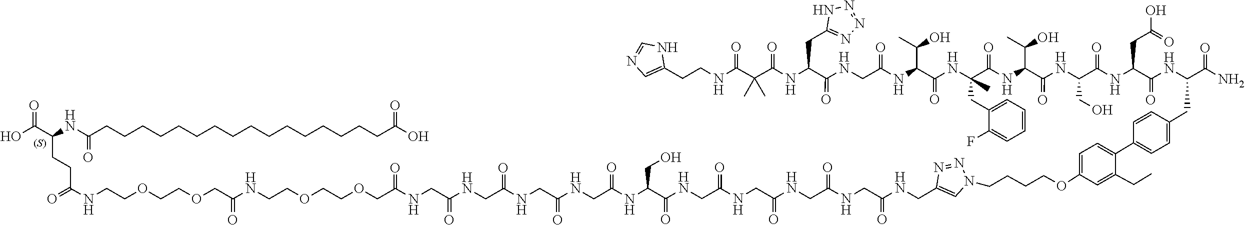 |
| LP44′a (SEQ ID NO: 318) | 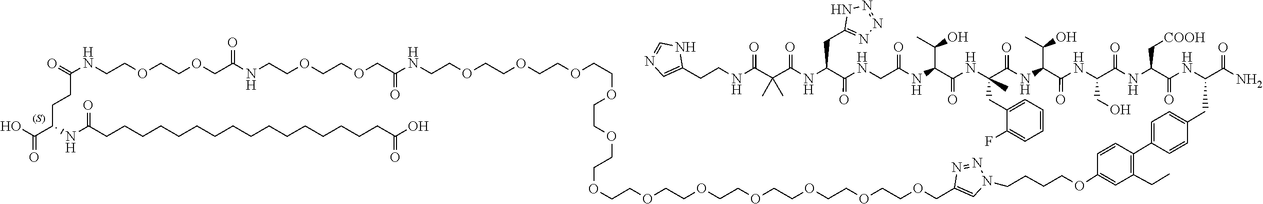 |
| LP45′a (SEQ ID NO: 319) | 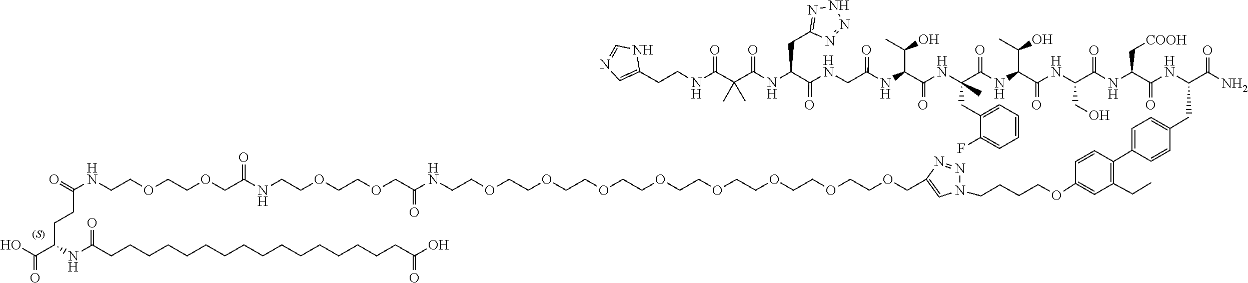 |
| LP46′a (SEQ ID NO: 320) | 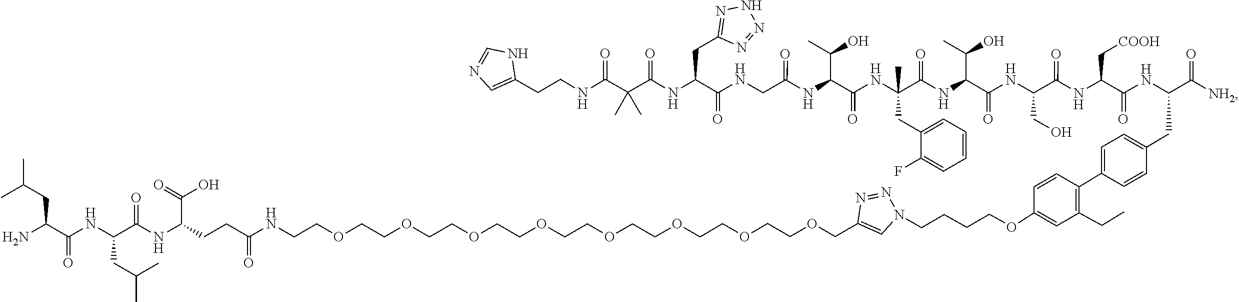 |

or a pharmaceutically acceptable salt thereof.

36. An antibody-thethered ligand (ATL) comprising an antibody or an antigen-binding fragment thereof conjugated to the compound of claim 1.

37. The ATL of claim 36, wherein said antibody or antigen-binding frament thereof is an anti-GLP1R antibody or antigen-binding fragment comprising REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280.

38. A compound having a structure of Formula (A):
BA-(L-P)ₘ (A),
wherein:
m ranges from about one to about 10;
BA is an antibody or an antigen-binding fragment thereof,
L is a linker;
P is a payload having the structure selected from the group consisting of: 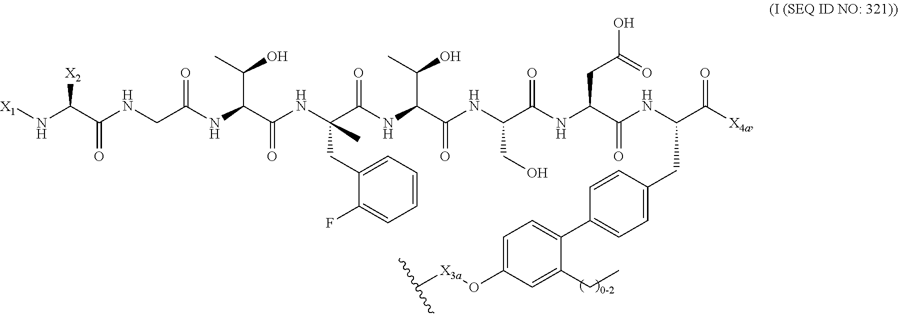
wherein 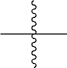
is the point of attachment of the payload to L;
X₁ is selected from H; 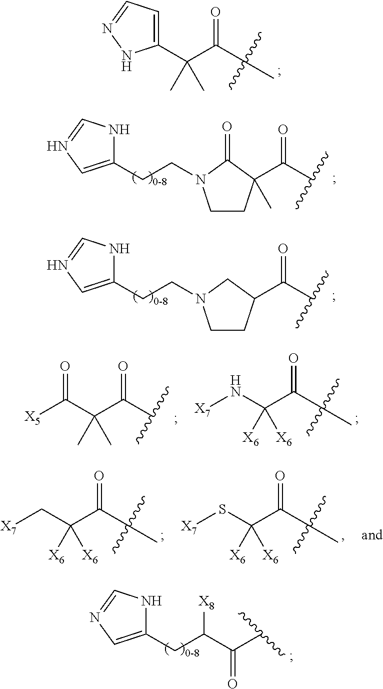
X₂ is selected from 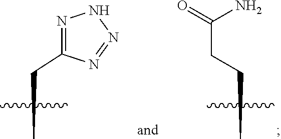
X₃ₐ is selected from bond, —CH₃, —(CH₂)₁₋₄(OCH₂CH₂)₂₋₁₅—NH₂, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
X_{3b} is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
Tr is a triazole moiety;
X₄ₐ is —NH₂ or —OH;
X₅ is selected from —OH, —NH₂, —NH—OH, and 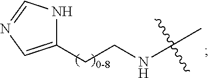
X₆ is independently at each occurrence selected from H, —OH, —CH₃, and —CH₂OH;
X₇ is selected from H, 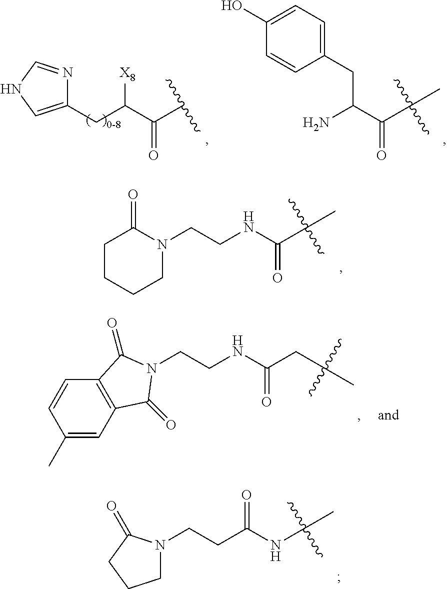
X₈ is selected from H, —OH, —NH₂, and 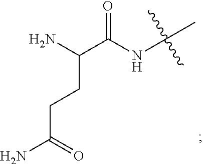 [SMILES: *NC(=O)C(N)CCC(N)=O]
Ar is selected from 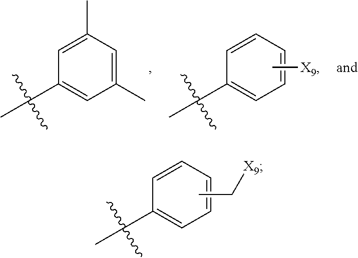
and
X₉ is selected from the group consisting of —NH₂ 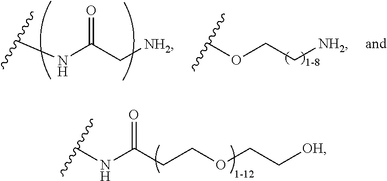
or a pharmaceutically acceptable salt thereof

39. A compound having a structure of Formula (B):
BA-L-P (B),
wherein:
BA is an antibody or an antigen-binding fragment thereof,
L is a linker;
P is a payload having the structure selected from the group consisting of: 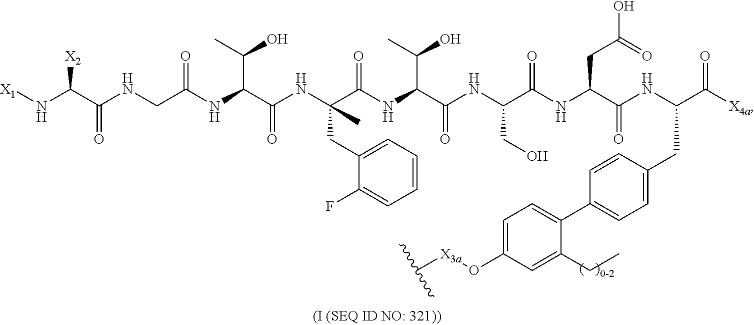
wherein 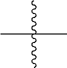
is the point of attachment of the payload to L;
X₁ is selected from H; 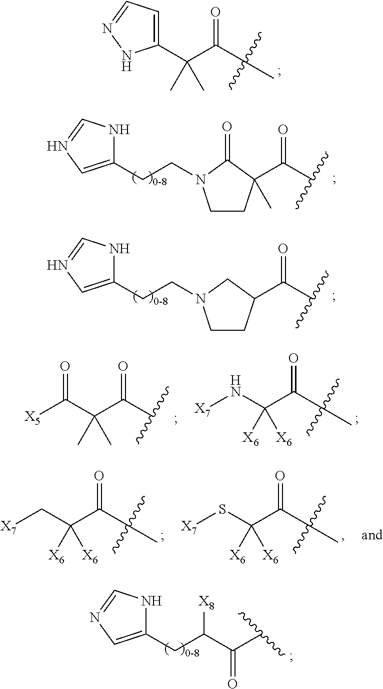
X₂ is selected from 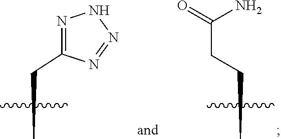
X₃ₐ is selected from bond, —CH₃, —(CH₂)₁₋₄(OCH₂CH₂)₂₋₁₅—NH₂, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
X_{3b} is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
Tr is a triazole moiety;
X₄ₐ is —NH₂ or —OH;
X₅ is selected from —OH, —NH₂, —NH—OH, and 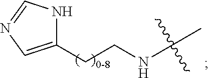
X₆ is independently at each occurrence selected from H, —OH, —CH₃, and —CH₂OH;
X₇ is selected from H, 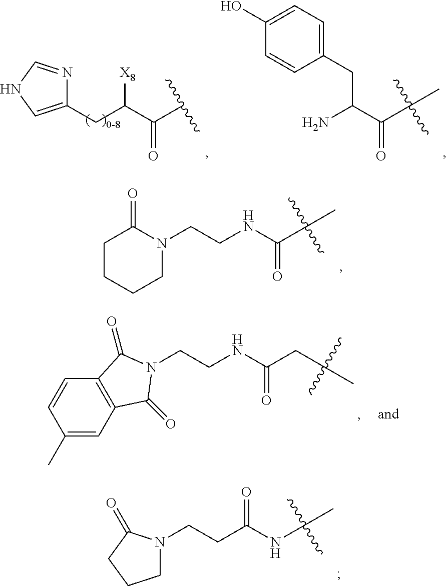
X₈ is selected from H, —OH, —NH₂, and 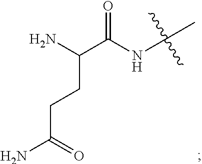 [SMILES: *NC(=O)C(N)CCC(N)=O]
Ar is selected from 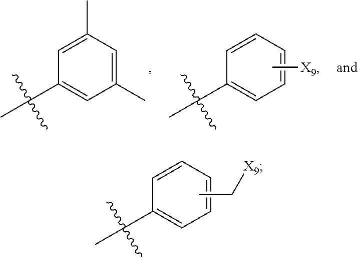
and
X₉ is selected from the group consisting of-NH₂, 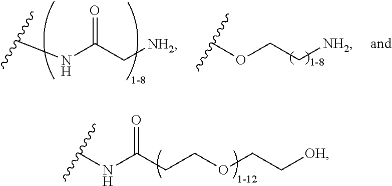
or a pharmaceutically acceptable salt thereof,

40. The compound of claim 38, wherein P has a structure of Formula (Ia): 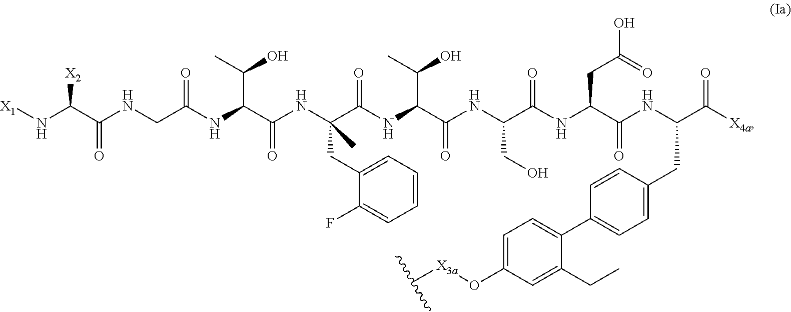
wherein
X₃ₐ is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—.

41. The compound of claim 38, wherein BA is a Glucagon-like peptide-1 receptor (GLP1R)-targeting antibody or an antigen-binding fragment thereof.

42.-57. (canceled)

58. The compound of claim 38, wherein m is about 1.

59. The compound of claim 38, wherein m ranges from about 1 to about 4.

60. (canceled)

61. The compound of claim 38, wherein the linker L has the structure of formula (L′):
-La-Y-Lp- (L′),
wherein La is a first linker covalently attached to the BA;
Y is a group comprising a triazole, and
Lp is absent or a second linker covalently attached to the P, wherein when Lp is absent, Y is also absent.

62.-67. (canceled)

68. The compound of claim 61, wherein Y-Lp has a structure selected from the group consisting of: 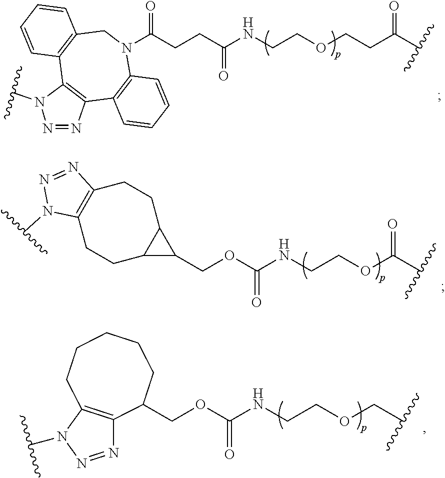
or a triazole regioisomer thereof,
wherein p is an integer from 1 to 36.

69.-73. (canceled)

74. The compound of claim 61, wherein the Lp is selected from the group consisting of Gly-Gly-Gly-Gly-Ser (G₄S) (SEQ ID NO: 1), Ser-Gly-Gly-Gly-Gly (SG₄) (SEQ ID NO: 2), and Gly-Gly-Gly-Gly-Ser-Gly-Gly-Gly-Gly-Ser (G₄S-G₄S) (SEQ ID NO: 3).

75.-77. (canceled)

78. The compound of claim 61, wherein Lp has a structure selected from the group consisting of: 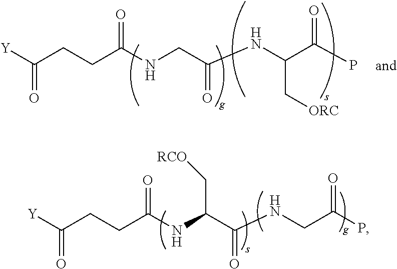
wherein Y is the group comprising a triazole and P is the payload, and wherein Rc is selected from H and glucose, g is an integer from 1 to 10 and s is an integer from 0 to 4.

79. The compound of claim 61, wherein Y-Lp has a structure selected from the group consisting of: 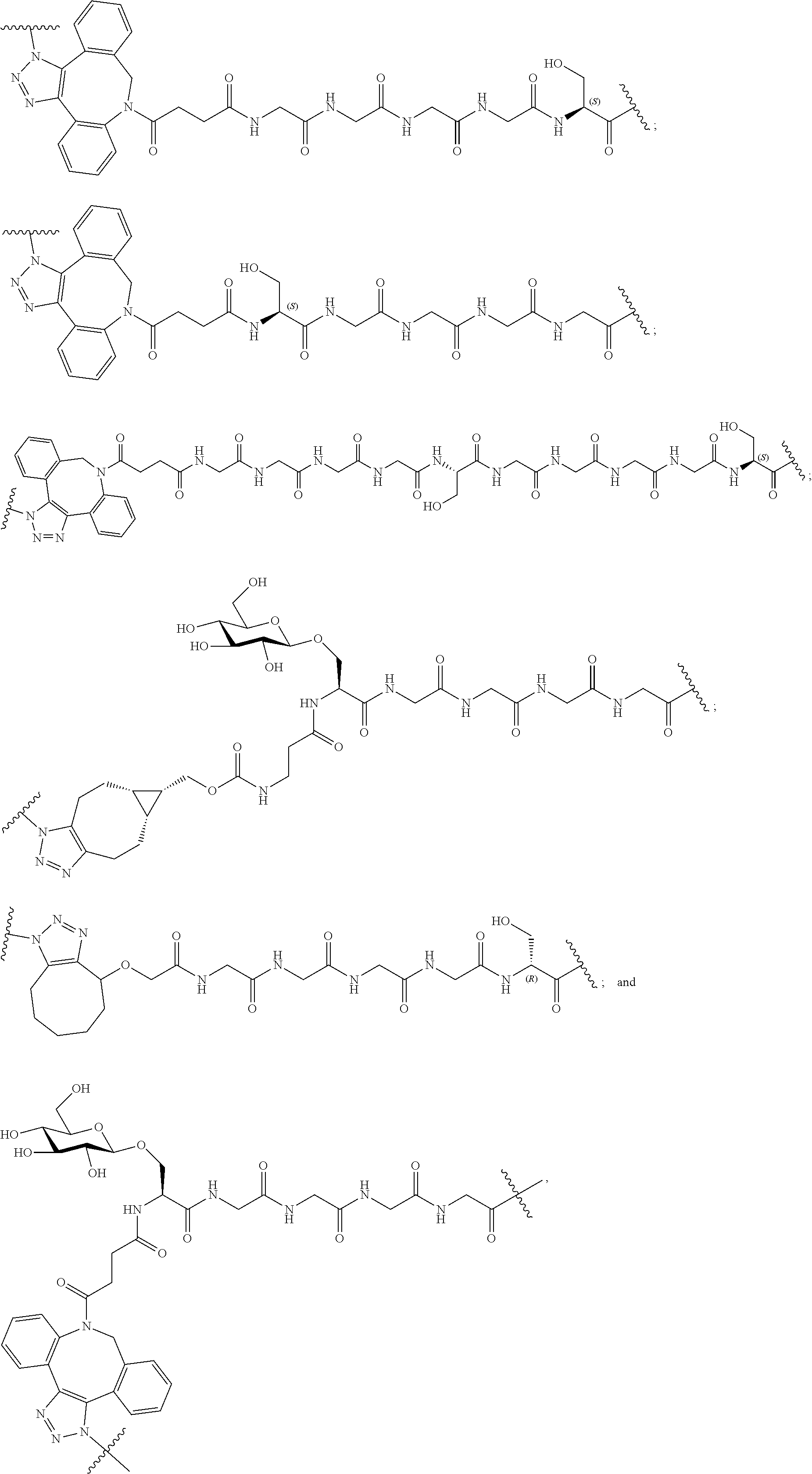
or a triazole regioisomer thereof.

80.-82. (canceled)

83. The compound of claim 61, wherein La has a structure selected from the group consisting of:

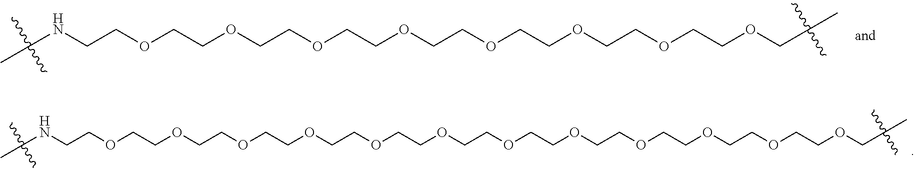 [SMILES: *COCCOCCOCCOCCOCCOCCOCCOCCN* ; *COCCOCCOCCOCCOCCOCCOCCOCCOCCOCCOCCOCCN*]

84.-86. (canceled)

87. A pharmaceutical composition comprising the compound of claim 38.

88. A pharmaceutical dosage form comprising the compound of claim 38.

89. A method of selectively targeting GLP1R on a surface of a cell with the compound according to claim 38.

90.-92. (canceled)

93. A method of enhancing GLP1R activity in an individual in need thereof comprising administering to the individual an effective amount of the compound of claim 38.

94. A method of lowering blood glucose levels in an individual in need thereof comprising administering to the individual an effective amount of the compound of claim 38.

95. A method of lowering body weight in an individual in need thereof comprising administering to the individual an effective amount of the compound of claim 38.

96. A method of treating a GLP1R-associated condition in an individual in need thereof comprising administering to the individual an effective amount of the compound of claim 38.

97.-99. (canceled)

100. A pharmaceutical composition comprising a compound having a structure of BA-(L-P)ₘ, or a pharmaceutically acceptable salt thereof, together with one or more pharmaceutically acceptable excipients, wherein:
m ranges from about one to about 10;
BA is an antibody or an antigen-binding fragment thereof,
L is a linker;
P is a payload having the structure selected from the group consisting of: 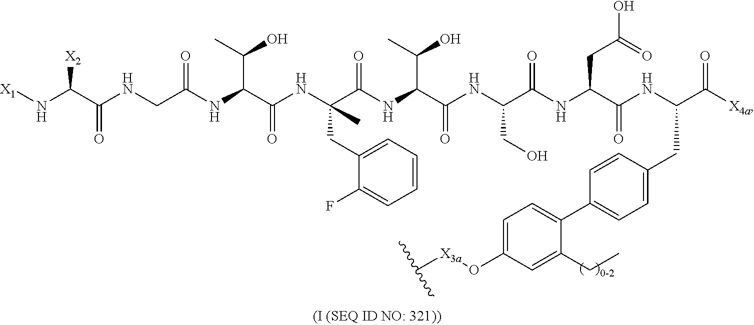
wherein 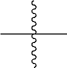
is the point of attachment of the payload to L;
X₁ is selected from H; 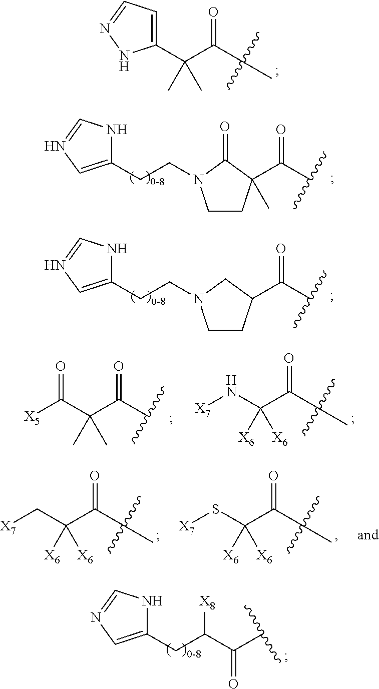
X₂ is selected from 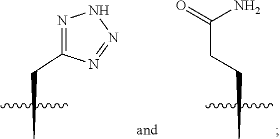
X₃ₐ is selected from bond, —CH₃, —(CH₂)₁₋₄(OCH₂CH₂)₂₋₁₅—NH₂, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
X_{3b} is selected from bond, —CH₃, —(CH₂)₂₋₆—NH₂, —(CH₂)₂₋₆—N₃, —(CH₂)₂₋₆-Tr-(CH₂)₁₋₆—NH₂, —(CH₂)₂₋₆—NH—, —(CH₂)₂₋₆-Tr-, and —(CH₂)₂₋₆-Tr-(CH₂)₁₋₆—NH—;
Tr is a triazole moiety;
X₄ₐ is —NH₂ or —OH;
X₅ is selected from —OH, —NH₂, —NH—OH, and 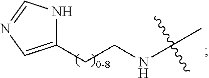
X₆ is independently at each occurrence selected from H, —OH, —CH₃, and —CH₂OH;
X₇ is selected from H, 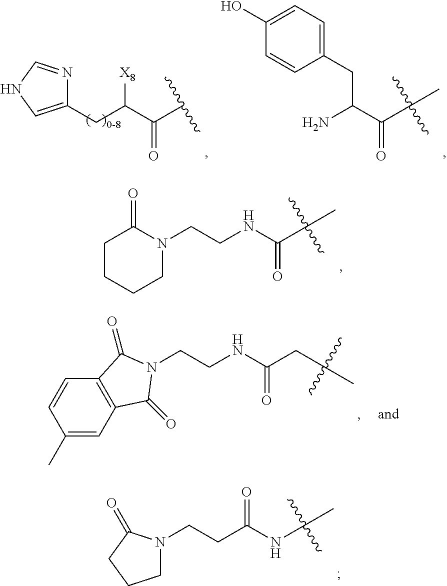
X₈ is selected from H, —OH, —NH₂, and 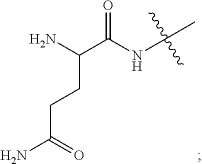 [SMILES: *NC(=O)C(N)CCC(N)=O]
Ar is selected from 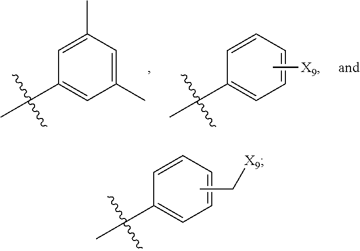
and
X₉ is selected from the group consisting of-NH₂, 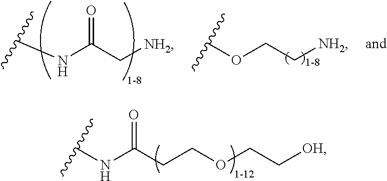
or a pharmaceutically acceptable salt thereof

101.-115. (canceled)

116. An antibody-thethered ligand (ATL) comprising an antibody or an antigen-binding fragment thereof conjugated to the compound of claim 11.

117. The ATL of claim 116, wherein said antibody or antigen-binding frament thereof is an anti-GLP1R antibody or antigen-binding fragment comprising REGN7990; REGN9268; REGN15869; REGN18121; REGN18123; REGN8070; REGN8072; REGN9267; REGN7988; REGN5619; REGN7989; REGN8069; REGN8071; REGN9426; REGN5203; REGN5204; REGN5617; REGN5619; REGN7987; REGN9270; REGN9278; REGN9279; or REGN9280.


SEQUENCE LISTING (354 sequences)

SEQ ID NO: 1 - protein, 5 aa, synthetic construct
GGGGS

SEQ ID NO: 2 - protein, 5 aa, synthetic construct
SGGGG

SEQ ID NO: 3 - protein, 10 aa, synthetic construct
GGGGSGGGGS

SEQ ID NO: 4 - protein, 5 aa, synthetic construct
LLQGG

SEQ ID NO: 5 - protein, 4 aa, synthetic construct
LLQG

SEQ ID NO: 6 - protein, 6 aa, synthetic construct
LSLSQG

SEQ ID NO: 7 - protein, 8 aa, synthetic construct
GGGLLQGG

SEQ ID NO: 8 - protein, 5 aa, synthetic construct
GLLQG

SEQ ID NO: 9 - protein, 10 aa, synthetic construct
GSPLAQSHGG

SEQ ID NO: 10 - protein, 7 aa, synthetic construct
GLLQGGG

SEQ ID NO: 11 - protein, 6 aa, synthetic construct
GLLQGG

SEQ ID NO: 12 - protein, 4 aa, synthetic construct
GLLQ

SEQ ID NO: 13 - protein, 8 aa, synthetic construct
LLQLLQGA

SEQ ID NO: 14 - protein, 5 aa, synthetic construct
LLQGA

SEQ ID NO: 15 - protein, 7 aa, synthetic construct
LLQYQGA

SEQ ID NO: 16 - protein, 6 aa, synthetic construct
LLQGSG

SEQ ID NO: 17 - protein, 6 aa, synthetic construct
LLQYQG

SEQ ID NO: 18 - protein, 7 aa, synthetic construct
LLQLLQG

SEQ ID NO: 19 - protein, 5 aa, synthetic construct
SLLQG

SEQ ID NO: 20 - protein, 5 aa, synthetic construct
LLQLQ

SEQ ID NO: 21 - protein, 6 aa, synthetic construct
LLQLLQ

SEQ ID NO: 22 - protein, 5 aa, synthetic construct
LLQGR

SEQ ID NO: 23 - DNA, 363 bp, synthetic construct
  source 1..363: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctca

SEQ ID NO: 24 - protein, 121 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSS

SEQ ID NO: 25 - DNA, 24 bp, synthetic construct
  source 1..24: mol_type = other DNA
ggattcacctttagcagctatgcc

SEQ ID NO: 26 - protein, 8 aa, synthetic construct
GFTFSSYA

SEQ ID NO: 27 - DNA, 24 bp, synthetic construct
  source 1..24: mol_type = other DNA
attagcggtagtggtggcagcaca

SEQ ID NO: 28 - protein, 8 aa, synthetic construct
ISGSGGST

SEQ ID NO: 29 - DNA, 42 bp, synthetic construct
  source 1..42: mol_type = other DNA
gcgaaaggccttatagcacctcgtccgatgggctttgactac

SEQ ID NO: 30 - protein, 14 aa, synthetic construct
AKGLIAPRPMGFDY

SEQ ID NO: 31 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaa

SEQ ID NO: 32 - protein, 107 aa, synthetic construct
DIQMTQSPSSVSASVGDRVTITCRASQGINSWLAWYQQKPGKAPKLLIYAASSLQSGVPSRFSGSGSGTDFTLTISSLQPEDFATYYCHQADSFPYTFGQGTKLEIK

SEQ ID NO: 33 - DNA, 18 bp, synthetic construct
  source 1..18: mol_type = other DNA
cagggtattaacagctgg

SEQ ID NO: 34 - protein, 6 aa, synthetic construct
QGINSW

SEQ ID NO: 35-36 - skipped (2 entries)

SEQ ID NO: 37 - DNA, 27 bp, synthetic construct
  source 1..27: mol_type = other DNA
caccaggctgacagtttcccgtacact

SEQ ID NO: 38 - protein, 9 aa, synthetic construct
HQADSFPYT

SEQ ID NO: 39 - DNA, 1347 bp, synthetic construct
  source 1..1347: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 40 - protein, 448 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 663 bp, synthetic construct
  source 1..663: mol_type = other DNA
ctgctgcaaggctctggcgacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 42 - protein, 220 aa, synthetic construct
LLQGSGDIQMTQSPSSVSASVGDRVTITCRASQGINSWLAWYQQKPGKAPKLLIYAASSLQSGVPSRFSGSGSGTDFTLTISSLQPEDFATYYCHQADSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 363 bp, synthetic construct; identical to SEQ ID NO: 23
  source 1..363: mol_type = other DNA

SEQ ID NO: 44 - protein, 121 aa, synthetic construct; identical to SEQ ID NO: 24

SEQ ID NO: 45 - DNA, 24 bp, synthetic construct
  source 1..24: mol_type = other DNA
ggattcacctttagcagctatgcc

SEQ ID NO: 46 - protein, 8 aa, synthetic construct
GFTFSSYA

SEQ ID NO: 47 - DNA, 24 bp, synthetic construct
  source 1..24: mol_type = other DNA
attagcggtagtggtggcagcaca

SEQ ID NO: 48 - protein, 8 aa, synthetic construct
ISGSGGST

SEQ ID NO: 49 - DNA, 42 bp, synthetic construct; identical to SEQ ID NO: 29
  source 1..42: mol_type = other DNA

SEQ ID NO: 50 - protein, 14 aa, synthetic construct
AKGLIAPRPMGFDY

SEQ ID NO: 51 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttggaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaa

SEQ ID NO: 52 - protein, 107 aa, synthetic construct
DIQMTQSPSSVSASVGDRVTITCRASQGINSWLAWYQQKPGKAPKLLIYAASSLESGVPSRFSGSGSGTDFTLTISSLQPEDFATYYCHQADSFPYTFGQGTKLEIK

SEQ ID NO: 53 - DNA, 18 bp, synthetic construct
  source 1..18: mol_type = other DNA
cagggtattaacagctgg

SEQ ID NO: 54 - protein, 6 aa, synthetic construct
QGINSW

SEQ ID NO: 55-56 - skipped (2 entries)

SEQ ID NO: 57 - DNA, 27 bp, synthetic construct
  source 1..27: mol_type = other DNA
caccaggctgacagtttcccgtacact

SEQ ID NO: 58 - protein, 9 aa, synthetic construct
HQADSFPYT

SEQ ID NO: 59 - DNA, 1347 bp, synthetic construct; identical to SEQ ID NO: 39
  source 1..1347: mol_type = other DNA

SEQ ID NO: 60 - protein, 448 aa, synthetic construct; identical to SEQ ID NO: 40

SEQ ID NO: 61 - DNA, 663 bp, synthetic construct
  source 1..663: mol_type = other DNA
ctgctgcaaggctctggcgacatccagatgacccagtctccatcttccgtgtctgcatctgtaggagacagagtcaccatcacttgtcgggcgagtcagggtattaacagctggttagcctggtatcagcagaaacctgggaaagcccctaagctcctgatctatgctgcatccagtttggaaagtggggtcccatcaaggttcagcggcagtggatctgggacagatttcactctcaccatcagcagcctgcagcctgaagattttgcaacttactattgtcaccaggctgacagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 62 - protein, 220 aa, synthetic construct
LLQGSGDIQMTQSPSSVSASVGDRVTITCRASQGINSWLAWYQQKPGKAPKLLIYAASSLESGVPSRFSGSGSGTDFTLTISSLQPEDFATYYCHQADSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 447 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - DNA, 1344 bp, synthetic construct
  source 1..1344: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 65 - protein, 446 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGLIAPRPMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 66 - DNA, 1341 bp, synthetic construct
  source 1..1341: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggaatgggtctcagctattagcggtagtggtggcagcacatactacgcagactccgtgaagggccggttcaccatctccagagacaattccaagaacacgctgtatctgcaaatgaacagcctgagagccgaggacacggccgtatattactgtgcgaaaggccttatagcacctcgtccgatgggctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggt

SEQ ID NO: 67 - protein, 8 aa, synthetic construct
  SITE 8: note = Residue attached to another sequence via an amide bond
GGSGGSGG

SEQ ID NO: 68 - protein, 6 aa, synthetic construct
LLQGSG

SEQ ID NO: 69 - protein, 6 aa, synthetic construct
  SITE 3: note = Residue side chain attached to another sequence
LLQGSG

SEQ ID NO: 70 - protein, 24 aa, synthetic construct
  SITE 3: note = Residue side chain attached to another sequence
LLQGSGDIQMTQSPSSVSASVGDR

SEQ ID NO: 71 - protein, 24 aa, synthetic construct
  SITE 3: note = Residue side chain attached to another sequence
LLQGSGDIQMTQSPSSVSASVGDR

SEQ ID NO: 72 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-methyl-4'-methoxy-[1, 1'-biphenyl]-4-yl)propanoic acid or Substituted (S)-2-amin o-3-(2'-methyl-4'-methoxy-[1,1'-biphenyl]-4-yl)propanamide
XGTXTSDX

SEQ ID NO: 73 - protein, 9 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid
  VARIANT 9: note = (S)-2-amino-5-(3,5-dimethylphenyl)pentanoic acid, substituted (S)-2-amino-5-phenylpentanoic acid, (S)-2-amino-5-(3,5-dimethylphenyl)pentanamide, or substituted (S)-2-amino-5-phenylpentanamide
XGTXTSDXX

SEQ ID NO: 74 - protein, 24 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  SITE 7: note = (S)-2-amino-3-(1H-tetrazol-5-yl)propanoic acid
  SITE 11: note = 2-amino-2-methylpropanoic acid
  SITE 13: note = (S)-2-amino-3-(1H-tetrazol-5-yl)propanoic acid
  VARIANT 18: note = Optionally substituted Ala
  SITE 20: note = (S)-2-amino-3-(2-fluorophenyl)-2-methylpropanoic acid
XGTXTSXYSIXLXKIAQXAXVQGG

SEQ ID NO: 75 - protein, 9 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 9: note = (S)-2-amino-5-(3,5-dimethylphenyl)pentanoic acid, or substituted (S)-2-amino-5-phenylpentanoic acid
XGTXTSDYX

SEQ ID NO: 76 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid or Substituted (S)-2-amino -3-(2'-ethyl-4'-methoxy-[1,1'-biphenyl]-4-yl)propanamide
XGTXTSDX

SEQ ID NO: 77 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-methyl-4'-methoxy-[1, 1'-biphenyl]-4-yl)propanoic acid or Substituted (S)-2-amin o-3-(2'-ethyl-4'-methoxy-[1,1'-biphenyl]-4-yl)propanamide
XGTXTSDX

SEQ ID NO: 78 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-methyl-4'-methoxy-[1, 1'-biphenyl]-4-yl)propanoic acid or Substituted (S)-2-amin o-3-(2'-methyl-4'-methoxy-[1,1'-biphenyl]-4-yl)propanamide
XGTXTSDX

SEQ ID NO: 79 - protein, 9 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid
  VARIANT 9: note = (S)-2-amino-5-(3,5-dimethylphenyl)pentanoic acid, or substituted (S)-2-amino-5-phenylpentanoic acid, or (S)-2-amino-5-(3,5-dimethylphenyl)pentanamide, or substituted (S)-2-amino-5-phenylpentanamide
XGTXTSDXX

SEQ ID NO: 80 - protein, 9 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid
  VARIANT 9: note = substituted (S)-2-amino-5-(4-methoxyphenyl)pentanoic acid or substituted (S)-2-amino-5-(4-methoxyphenyl)pentanamide
XGTXTSDXX

SEQ ID NO: 81 - protein, 24 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  SITE 7: note = (S)-2-amino-3-(1H-tetrazol-5-yl)propanoic acid
  SITE 11: note = 2-amino-2-methylpropanoic acid
  SITE 13: note = (S)-2-amino-3-(1H-tetrazol-5-yl)propanoic acid
  VARIANT 18: note = substituted Ala
  SITE 20: note = (S)-2-amino-3-(2-fluorophenyl)-2-methylpropanoic acid
XGTXTSXYSIXLXKIAQXAXVQGG

SEQ ID NO: 82 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid or substituted (S)-2-amino -3-(2'-ethyl-4'-methoxy-[1,1'-biphenyl]-4-yl)propanamide
XGTXTSDX

SEQ ID NO: 83 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-methyl-4'-methoxy-[1, 1'-biphenyl]-4-yl)propanoic acid or Substituted (S)-2-amin o-3-(2'-methyl-4'-methoxy-[1,1'-biphenyl]-4-yl)propanamide
XGTXTSDX

SEQ ID NO: 84 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-15-oxo-3,6, 9,12-tetraoxa-16-azaicosan-20-yl)oxy)-2'-ethyl-[1,1'-biphe nyl]-4-yl)propanoic acid
  SITE 10: note = (S)-2-amino-5-(3,5-dimethylphenyl)pentanoic acid
XXGTXTSDXX

SEQ ID NO: 85 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-27-oxo-3,6, 9,12,15,18,21,24-octaoxa-28-azadotriacontan-32-yl)oxy)-2'- ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 86 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-39-oxo-3,6, 9,12,15,18,21,24,27,30,33,36-dodecaoxa-40-azatetratetracon tan-44-yl)oxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 87 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
  SITE 10: note = (S)-2-amino-5-(3,5-dimethylphenyl)pentanoic acid
XXGTXTSDXX

SEQ ID NO: 88 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-((1-((1R,8S,9s)-bicyclo[6.1.0]non -4-yn-9-yl)-3,17-dioxo-2,7,10,13,16-pentaoxa-4,18-diazadoc osan-22-yl)oxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 89 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 90 - protein, 5 aa, synthetic construct
  SITE 5: note = Residue attached to another sequence via an amide bond
GGGGS

SEQ ID NO: 91 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 92 - protein, 5 aa, synthetic construct
  SITE 5: note = Residue attached to another sequence via an amide bond
SGGGG

SEQ ID NO: 93 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(1-((1R,8S,9s)-bicyclo[6.1. 0]non-4-yn-9-yl)-3-oxo-2,7,10,13,16-pentaoxa-4-azaheptadec an-17-yl)-1H-1,2,3-triazol-1-yl)butoxy)-2'-ethyl-[1,1'-bip henyl]-4-yl)propanoic acid
  SITE 10: note = (S)-2-amino-5-(3,5-dimethylphenyl)pentanoic acid
XXGTXTSDXX

SEQ ID NO: 94 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (2S)-2-amino-3-(4'-(4-(4-(16-(cyclooct-2-yn-1-yloxy )-15-oxo-2,5,8,11-tetraoxa-14-azahexadecyl)-1H-1,2,3-triaz ol-1-yl)butoxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 95 - protein, 9 aa, synthetic construct
  SITE 1: note = (S)-2-(6-(1H-imidazol-5-yl)hexanamido)-3-(2H-tetraz ol-5-yl)propanoic acid
  SITE 4: note = (S)-2-amino-3-(2-fluorophenyl)-2-methylpropanoic acid
  VARIANT 8: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
  SITE 9: note = (S)-2-amino-5-(3,5-dimethylphenyl)pentanoic acid
XGTXTSDXX

SEQ ID NO: 96 - protein, 9 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  VARIANT 8: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
  SITE 9: note = (S)-2-amino-5-(3,5-dimethylphenyl)pentanoic acid
XGTXTSDXX

SEQ ID NO: 97 - protein, 9 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid side chain attached to another sequence via an amide bond
  SITE 9: note = (S)-2-amino-5-(3,5-dimethylphenyl)pentanoic acid
XGTXTSDXX

SEQ ID NO: 98 - protein, 5 aa, synthetic construct
  SITE 5: note = Residue attached to another sequence via an amide bond
GGGGS

SEQ ID NO: 99 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 100 - protein, 10 aa, synthetic construct
  SITE 10: note = Residue attached to another sequence via an amide bond
GGGGSGGGGS

SEQ ID NO: 101 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 102 - protein, 6 aa, synthetic construct
  SITE 1: note = 3-(((((1R,8S,9s)-bicyclo[6.1.0]non-4-yn-9-yl)methox y)carbonyl)amino)propanoic acid
  SITE 2: note = O-((2R,3R,4S,5S,6R)-3,4,5-trihydroxy-6-(hydroxymeth yl)tetrahydro-2H-pyran-2-yl)-L-serine
  SITE 6: note = Residue attached to another sequence via an amide bond
XXGGGG

SEQ ID NO: 103 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 104 - protein, 5 aa, synthetic construct
  SITE 1: note = (2-(cyclooct-2-yn-1-yloxy)acetyl)glycine
  SITE 5: note = Residue attached to another sequence via an amide bond
XGGGS

SEQ ID NO: 105 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 106 - protein, 5 aa, synthetic construct
  SITE 1: note = O-((2R,3R,4S,5S,6R)-3,4,5-trihydroxy-6-(hydroxymeth yl)tetrahydro-2H-pyran-2-yl)-L-serine
  SITE 5: note = Residue attached to another sequence via an amide bond
XGGGG

SEQ ID NO: 107 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid
  SITE 10: note = (S)-2-amino-5-(4-((1-amino-75-oxo-3,6,9,12,15,18,21 ,24,27,30,33,36,39,42,45,48,51,54,57,60,63,66,69,72-tetrac osaoxa-76-azaoctacontan-80-yl)oxy)phenyl)pentanoic acid
XXGTXTSDXX

SEQ ID NO: 108 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
  SITE 10: note = (S)-2-amino-5-(3,5-dimethylphenyl)pentanoic acid
XXGTXTSDXX

SEQ ID NO: 109 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid
  SITE 10: note = (S)-2-amino-5-(4-((1-amino-75-oxo-3,6,9,12,15,18,21 ,24,27,30,33,36,39,42,45,48,51,54,57,60,63,66,69,72-tetrac osaoxa-76-azaoctacontan-80-yl)oxy)phenyl)pentanoic acid
XXGTXTSDXX

SEQ ID NO: 110 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-(4-(4-(1-((5aS,6R,6aR)-1 ,4,5,5a,6,6a,7,8-octahydrocyclopropa[5,6]cycloocta[1,2-d][ 1,2,3]triazol-6-yl)-3-oxo-2,7,10,13,16-pentaoxa-4-azahepta decan-17-yl)-1H-1,2,3-triazol-1-yl)butoxy)-[1,1'-biphenyl] -4-yl)propanoic acid
  SITE 10: note = (S)-2-amino-5-(3,5-dimethylphenyl)pentanoic acid
XXGTXTSDXX

SEQ ID NO: 111 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-((1-((5aS,6R,6aR)-1,4,5, 5a,6,6a,7,8-octahydrocyclopropa[5,6]cycloocta[1,2-d][1,2,3 ]triazol-6-yl)-3,17-dioxo-2,7,10,13,16-pentaoxa-4,18-diaza docosan-22-yl)oxy)-[1,1'-biphenyl]-4-yl)propanoic acid
  SITE 10: note = (S)-2-amino-5-(3,5-dimethylphenyl)pentanoic acid
XXGTXTSDXX

SEQ ID NO: 112 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 113 - protein, 5 aa, synthetic construct
  SITE 1: note = (7-(3,9-dihydro-8H-dibenzo[b,f][1,2,3]triazolo[4,5- d]azocin-8-yl)-4,7-dioxoheptanoyl)glycine
  SITE 5: note = Residue attached to another sequence via an amide bond
XGGGS

SEQ ID NO: 114 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-(4-(4-(25-amino-2,5,8 ,11,14,17,20,23-octaoxapentacosyl)-1H-1,2,3-triazol-1-yl)b utoxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 115 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(30-(3,9-dihydro-8H-dibenzo [b,f][1,2,3]triazolo[4,5-d]azocin-8-yl)-27,30-dioxo-2,5,8, 11,14,17,20,23-octaoxa-26-azatriacontyl)-1H-1,2,3-triazol- 1-yl)butoxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 116 - protein, 11 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 11: note = (S)-2-amino-5-(3,5-dimethylphenyl)pentanoic acid
YXXGTXTSDXX

SEQ ID NO: 117 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(4-(29-hydroxy-3-oxo-6,9,12,15,18,21, 24,27-octaoxa-2-azanonacosyl)phenyl)pentanoic acid
XXGTXTSDXX

SEQ ID NO: 118 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(37-amino-2,5,8,11,14,17,20 ,23,26,29,32,35-dodecaoxaheptatriacontyl)-1H-1,2,3-triazol -1-yl)butoxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 119 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 120 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-30-amino-1-(1-(4-((4'-((S)-2-amino-2-carboxyeth yl)-2-ethyl-[1,1'-biphenyl]-4-yl)oxy)butyl)-1H-1,2,3-triaz ol-4-yl)-27-oxo-2,5,8,11,14,17,20,23-octaoxa-26-azahentria contan-31-oic acid
  SITE 10: note = (S)-2-amino-5-(3,5-dimethylphenyl)pentanoic acid
XXGTXTSDXX

SEQ ID NO: 121 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-1-(1-(4-((4'-(2-amino-2-carboxyethyl)-2-ethyl-[ 1,1'-biphenyl]-4-yl)oxy)butyl)-1H-1,2,3-triazol-4-yl)-2,5, 8,11,14,17,20,23-octaoxahexacosan-26-oic acid side chain attached to another sequence via an amide bond
  SITE 10: note = (S)-2-amino-5-(3,5-dimethylphenyl)pentanoic acid
XXGTXTSDXX

SEQ ID NO: 122 - protein, 6 aa, synthetic construct
  SITE 1: note = Residue attached to another sequence via an amide bond
GGTEPL

SEQ ID NO: 123 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = ((S)-2-amino-3-(4'-(4-(4-(25-amino-2,5,8,11,14,17,2 0,23-octaoxapentacosyl)-1H-1,2,3-triazol-1-yl)butoxy)-2'-e thyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 124 - protein, 6 aa, synthetic construct
  SITE 1: note = ((benzyloxy)carbonyl)-L-leucine
  SITE 3: note = Residue side chain attached to another sequence via an amide bond
XLQGSG

SEQ ID NO: 125 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 126 - protein, 5 aa, synthetic construct
  SITE 5: note = Residue attached to another sequence via an amide bond
GGGGS

SEQ ID NO: 127 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 128 - protein, 5 aa, synthetic construct
  SITE 5: note = Residue attached to another sequence via an amide bond
SGGGG

SEQ ID NO: 129 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 130 - protein, 8 aa, synthetic construct
  SITE 8: note = Residue attached to another sequence via an amide bond
GGSGGSGG

SEQ ID NO: 131 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 132 - protein, 9 aa, synthetic construct
  SITE 9: note = Residue attached to another sequence via an amide bond
GGGGSGGGG

SEQ ID NO: 133 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 134 - protein, 14 aa, synthetic construct
  SITE 14: note = Residue attached to another sequence via an amide bond
GGGGSGGGGSGGGG

SEQ ID NO: 135 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 136 - protein, 11 aa, synthetic construct
  SITE 11: note = Residue attached to another sequence via an amide bond
GGSGGSGGSGG

SEQ ID NO: 137 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 138 - protein, 5 aa, synthetic construct
  SITE 5: note = Residue attached to another sequence via an amide bond
GGGGS

SEQ ID NO: 139 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
  SITE 10: note = (S)-2-amino-5-(3,5-dimethylphenyl)pentanoic acid
XXGTXTSDXX

SEQ ID NO: 140 - protein, 9 aa, synthetic construct
  SITE 9: note = Residue attached to another sequence via an amide bond
GGGGSGGGG

SEQ ID NO: 141 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(55-amino-39,48-dioxo-2,5,8 ,11,14,17,20,23,26,29,32,35,41,44,50,53-hexadecaoxa-38,47- diazapentapentacontyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 142 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(43-amino-27,36-dioxo-2,5,8 ,11,14,17,20,23,29,32,38,41-dodecaoxa-26,35-diazatritetrac ontyl)-1H-1,2,3-triazol-1-yl)butoxy)-2'-ethyl-[1,1'-biphen yl]-4-yl)propanoic acid
  SITE 10: note = (S)-2-amino-5-(4-(29-hydroxy-3-oxo-6,9,12,15,18,21, 24,27-octaoxa-2-azanonacosyl)phenyl)pentanoic acid
XXGTXTSDXX

SEQ ID NO: 143 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 144 - protein, 9 aa, synthetic construct
  VARIANT 1: note = Substituted 2-butyramido-2-methylpropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-methoxy-2'-methyl-[1, 1'-biphenyl]-4-yl)propanoic acid or substituted (S)-2-amin o-3-(4'-methoxy-2'-methyl-[1,1'-biphenyl]-4-yl)propanamide
XXGTXTSDX

SEQ ID NO: 145 - protein, 9 aa, synthetic construct
  VARIANT 1: note = Substituted 2-butyramido-2-methylpropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-methoxy-2'-methyl-[1, 1'-biphenyl]-4-yl)propanoic acid or substituted (S)-2-amin o-3-(4'-methoxy-2'-methyl-[1,1'-biphenyl]-4-yl)propanamide
XXGTXTSDX

SEQ ID NO: 146 - protein, 9 aa, synthetic construct
  SITE 1: note = N1-(2-(1H-imidazol-5-yl)ethyl)-2,2-dimethylmalonami de
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
XXGTXTSDX

SEQ ID NO: 147 - protein, 9 aa, synthetic construct
  SITE 1: note = (S)-2-amino-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-azidobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 148 - protein, 9 aa, synthetic construct
  SITE 1: note = (S)-2-amino-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 149 - protein, 9 aa, synthetic construct
  SITE 1: note = (S)-3-((1-carboxy-2-(2H-tetrazol-5-yl)ethyl)amino)- 2,2-dimethyl-3-oxopropanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 150 - protein, 9 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 151 - protein, 9 aa, synthetic construct
  SITE 1: note = (S)-2-(3-(hydroxyamino)-2,2-dimethyl-3-oxopropanami do)-3-(2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 152 - protein, 9 aa, synthetic construct
  SITE 1: note = (S)-2-(2-methyl-2-(1H-pyrazol-5-yl)propanamido)-3-( 2H-tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 153 - protein, 10 aa, synthetic construct
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
SXGTXTSDXX

SEQ ID NO: 154 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 155 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 156 - protein, 11 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanoic acid
HAXGTXTSDXX

SEQ ID NO: 157 - protein, 9 aa, synthetic construct
  SITE 1: note = (S)-2-(6-(1H-imidazol-5-yl)hexanamido)-3-(2H-tetraz ol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 158 - protein, 10 aa, synthetic construct
  SITE 1: note = (3-(1H-imidazol-4-yl)propanoyl)glycine
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 159 - protein, 12 aa, synthetic construct
  SITE 4: note = (S)-2-amino-3-(2H-tetrazol-5-yl)propanoic acid
  SITE 7: note = (S)-2-amino-3-(2-fluorophenyl)-2-methylpropanoic acid
  SITE 11: note = (S)-2-amino-3-(4'-(4-aminobutoxy)-2'-ethyl-[1,1'-bi phenyl]-4-yl)propanoic acid
  SITE 12: note = (S)-2-amino-5-(3,5-dimethylphenyl)pentanoic acid
QHAXGTXTSDXX

SEQ ID NO: 160 - protein, 10 aa, synthetic construct
  SITE 1: note = (R)-1-(2-(1H-imidazol-5-yl)ethyl)-3-methyl-2-oxopyr rolidine-3-carboxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 161 - protein, 10 aa, synthetic construct
  SITE 1: note = (S)-1-(2-(1H-imidazol-5-yl)ethyl)-3-methyl-2-oxopyr rolidine-3-carboxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 162 - protein, 10 aa, synthetic construct
  SITE 1: note = (S)-1-(2-(1H-imidazol-5-yl)ethyl)pyrrolidine-3-carb oxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 163 - protein, 10 aa, synthetic construct
  SITE 1: note = (R)-1-(2-(1H-imidazol-5-yl)ethyl)pyrrolidine-3-carb oxylic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 164 - protein, 11 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanoic acid
YAXGTXTSDXX

SEQ ID NO: 165 - protein, 11 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-aminobutoxy)-2'-ethyl-[1,1'-bi phenyl]-4-yl)propanoic acid
  SITE 11: note = (S)-2-amino-5-(3,5-dimethylphenyl)pentanoic acid
YXXGTXTSDXX

SEQ ID NO: 166 - protein, 9 aa, synthetic construct
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid
  SITE 9: note = (S)-2-amino-5-(3,5-dimethylphenyl)pentanoic acid
QGTXTSDXX

SEQ ID NO: 167 - protein, 10 aa, synthetic construct
  SITE 1: note = 3-((2-(1H-imidazol-5-yl)ethyl)amino)-2,2-dimethyl-3 -oxopropanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
  SITE 10: note = (S)-2-amino-5-(3,5-dimethylphenyl)pentanoic acid
XQGTXTSDXX

SEQ ID NO: 168 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-methoxy-[1,1'-biphenyl]- 4-yl)propanoic acid
  SITE 10: note = (S)-2-amino-5-(4-(4-aminobutoxy)phenyl)pentanoic acid
XXGTXTSDXX

SEQ ID NO: 169 - protein, 10 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 170 - protein, 10 aa, synthetic construct
  SITE 1: note = 2-(4-(1H-imidazol-4-yl)butanamido)-2-methylpropanoi c acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 171 - protein, 10 aa, synthetic construct
  SITE 1: note = 2-(5-(1H-imidazol-4-yl)pentanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 172 - protein, 10 aa, synthetic construct
  SITE 1: note = 2-(6-(1H-imidazol-4-yl)hexanamido)-2-methylpropanoi c acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 173 - protein, 10 aa, synthetic construct
  SITE 1: note = 2-(7-(1H-imidazol-4-yl)heptanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 174 - protein, 10 aa, synthetic construct
  SITE 1: note = 2-(8-(1H-imidazol-5-yl)octanamido)-2-methylpropanoi c acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 175 - protein, 10 aa, synthetic construct
  SITE 1: note = 2-(9-(1H-imidazol-5-yl)nonanamido)-2-methylpropanoi c acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 176 - protein, 4 aa, synthetic construct
  SITE 4: note = Residue attached to another sequence via an amide bond
GGGG

SEQ ID NO: 177 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(aminomethyl)phenyl)pentanoic acid side chain attached to another sequence via an amide bond
XXGTXTSDXX

SEQ ID NO: 178 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(17-hydroxy-3-oxo-6,9,12,15-tetrao xa-2-azaheptadecyl)phenyl)pentanoic acid
XXGTXTSDXX

SEQ ID NO: 179 - protein, 8 aa, synthetic construct
  SITE 8: note = Residue attached to another sequence via an amide bond
GGGGGGGG

SEQ ID NO: 180 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(aminomethyl)phenyl)pentanoic acid side chain attached to another sequence via an amide bond
XXGTXTSDXX

SEQ ID NO: 181 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(4-(29-hydroxy-3-oxo-6,9,12,15,18,21, 24,27-octaoxa-2-azanonacosyl)phenyl)pentanoic acid
XXGTXTSDXX

SEQ ID NO: 182 - protein, 10 aa, synthetic construct
  SITE 1: note = 2,2-dimethyl-4-oxo-4-((2-(2-oxopiperidin-1-yl)ethyl )amino)butanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 183 - protein, 9 aa, synthetic construct
  SITE 1: note = (S)-2-(2-((2-((2-(5-methyl-1,3-dioxoisoindolin-2-yl )ethyl)amino)-2-oxoethyl)thio)acetamido)-3-(2H-tetrazol-5- yl)propanoic acid
  SITE 4: note = (S)-2-amino-3-(2-fluorophenyl)-2-methylpropanoic acid
  SITE 8: note = (S)-2-amino-3-(4'-(4-azidobutoxy)-2'-ethyl-[1,1'-bi phenyl]-4-yl)propanoic acid
  SITE 9: note = (S)-2-amino-5-(3,5-dimethylphenyl)pentanoic acid
XGTXTSDXX

SEQ ID NO: 184 - protein, 10 aa, synthetic construct
  SITE 1: note = 3-(3-(2-oxopyrrolidin-1-yl)propanamido)propanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTSDXX

SEQ ID NO: 185 - protein, 11 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 11: note = (S)-2-amino-5-(4-(aminomethyl)phenyl)pentanoic acid
YXXGTXTSDYX

SEQ ID NO: 186 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 187 - protein, 25 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 8: note = (S)-2-amino-3-(2H-tetrazol-5-yl)propanoic acid
  SITE 12: note = 2-amino-2-methylpropanoic acid
  SITE 14: note = (S)-2-amino-3-(2H-tetrazol-5-yl)propanoic acid
  SITE 19: note = N6-diazo-L-lysine
  SITE 21: note = (S)-2-amino-3-(2-fluorophenyl)-2-methylpropanoic acid
XXGTXTSXYSIXLXKIAQXAXVQGG

SEQ ID NO: 188 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanamide
XXGTXTSDX

SEQ ID NO: 189 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-azidobutoxy)-2'-ethyl-[1,1'-bi phenyl]-4-yl)propanoic acid
XXGTXTSDX

SEQ ID NO: 190 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide
XXGTXTSDX

SEQ ID NO: 191 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid
XXGTXTSDX

SEQ ID NO: 192 - protein, 8 aa, synthetic construct
  SITE 1: note = (S)-3-((1-carboxy-2-(2H-tetrazol-5-yl)ethyl)amino)- 2,2-dimethyl-3-oxopropanoic acid
  SITE 4: note = (S)-2-amino-3-(2-fluorophenyl)-2-methylpropanoic acid
  VARIANT 8: note = (S)-2-amino-3-(4'-(4-aminobutoxy)-2'-ethyl-[1,1'-bi phenyl]-4-yl)propanoic acid or (S)-2-amino-3-(4'-(4-azidob utoxy)-2'-ethyl-[1,1'-biphenyl]-4-yl)propanoic acid
XGTXTSDX

SEQ ID NO: 193 - protein, 8 aa, synthetic construct
  SITE 1: note = (S)-3-((1-carboxy-2-(2H-tetrazol-5-yl)ethyl)amino)- 2,2-dimethyl-3-oxopropanoic acid
  SITE 4: note = (S)-2-amino-3-(2-fluorophenyl)-2-methylpropanoic acid
  VARIANT 8: note = (S)-2-amino-3-(4'-(4-aminobutoxy)-2'-ethyl-[1,1'-bi phenyl]-4-yl)propanamide or (S)-2-amino-3-(4'-(4-azidobuto xy)-2'-ethyl-[1,1'-biphenyl]-4-yl)propanamide
XGTXTSDX

SEQ ID NO: 194 - protein, 9 aa, synthetic construct
  SITE 1: note = 3-amino-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-(4-aminobutoxy)-2'-ethyl-[1,1'-bi phenyl]-4-yl)propanoic acid or (S)-2-amino-3-(4'-(4-azidob utoxy)-2'-ethyl-[1,1'-biphenyl]-4-yl)propanoic acid
XXGTXTSDX

SEQ ID NO: 195 - protein, 9 aa, synthetic construct
  SITE 1: note = 3-amino-2,2-dimethyl-3-oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-(4-aminobutoxy)-2'-ethyl-[1,1'-bi phenyl]-4-yl)propanamide or (S)-2-amino-3-(4'-(4-azidobuto xy)-2'-ethyl-[1,1'-biphenyl]-4-yl)propanamide
XXGTXTSDX

SEQ ID NO: 196 - protein, 8 aa, synthetic construct
  SITE 1: note = (S)-2-(2-methyl-2-(1H-pyrazol-5-yl)propanamido)-3-( 2H-tetrazol-5-yl)propanoic acid
  SITE 4: note = (S)-2-amino-3-(2-fluorophenyl)-2-methylpropanoic acid
  VARIANT 8: note = (S)-2-amino-3-(4'-(4-aminobutoxy)-2'-ethyl-[1,1'-bi phenyl]-4-yl)propanoic acid or (S)-2-amino-3-(4'-(4-azidob utoxy)-2'-ethyl-[1,1'-biphenyl]-4-yl)propanoic acid
XGTXTSDX

SEQ ID NO: 197 - protein, 8 aa, synthetic construct
  SITE 1: note = (S)-2-(2-methyl-2-(1H-pyrazol-5-yl)propanamido)-3-( 2H-tetrazol-5-yl)propanoic acid
  SITE 4: note = (S)-2-amino-3-(2-fluorophenyl)-2-methylpropanoic acid
  VARIANT 8: note = (S)-2-amino-3-(4'-(4-aminobutoxy)-2'-ethyl-[1,1'-bi phenyl]-4-yl)propanamide or (S)-2-amino-3-(4'-(4-azidobuto xy)-2'-ethyl-[1,1'-biphenyl]-4-yl)propanamide
XGTXTSDX

SEQ ID NO: 198 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-(4-aminobutoxy)-2'-ethyl-[1,1'-bi phenyl]-4-yl)propanoic acid or (S)-2-amino-3-(4'-(4-azidob utoxy)-2'-ethyl-[1,1'-biphenyl]-4-yl)propanoic acid
XXGTXTSDX

SEQ ID NO: 199 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-(4-aminobutoxy)-2'-ethyl-[1,1'-bi phenyl]-4-yl)propanamide or (S)-2-amino-3-(4'-(4-azidobuto xy)-2'-ethyl-[1,1'-biphenyl]-4-yl)propanamide
XXGTXTSDX

SEQ ID NO: 200 - protein, 10 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  VARIANT 10: note = (S)-2-amino-3-(4'-(4-aminobutoxy)-2'-ethyl-[1,1'-bi phenyl]-4-yl)propanoic acid or (S)-2-amino-3-(4'-(4-azidob utoxy)-2'-ethyl-[1,1'-biphenyl]-4-yl)propanoic acid
HAXGTXTSDX

SEQ ID NO: 201 - protein, 10 aa, synthetic construct
  SITE 3: note = (S)-2-amino-3-(2H-tetrazol-5-yl)propanoic acid
  SITE 6: note = (S)-2-amino-3-(2-fluorophenyl)-2-methylpropanoic acid
  VARIANT 10: note = (S)-2-amino-3-(4'-(4-aminobutoxy)-2'-ethyl-[1,1'-bi phenyl]-4-yl)propanamide or (S)-2-amino-3-(4'-(4-azidobuto xy)-2'-ethyl-[1,1'-biphenyl]-4-yl)propanamide
HAXGTXTSDX

SEQ ID NO: 202 - protein, 9 aa, synthetic construct
  VARIANT 1: note = Substituted butyrylglycine
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-(4-aminobutoxy)-2'-ethyl-[1,1'-bi phenyl]-4-yl)propanoic acid or (S)-2-amino-3-(4'-(4-azidob utoxy)-2'-ethyl-[1,1'-biphenyl]-4-yl)propanoic acid
XXGTXTSDX

SEQ ID NO: 203 - protein, 9 aa, synthetic construct
  VARIANT 1: note = Substituted butyrylglycine
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-(4-aminobutoxy)-2'-ethyl-[1,1'-bi phenyl]-4-yl)propanamide or (S)-2-amino-3-(4'-(4-azidobuto xy)-2'-ethyl-[1,1'-biphenyl]-4-yl)propanamide
XXGTXTSDX

SEQ ID NO: 204 - protein, 9 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-methoxy-2'-methyl-[1, 1'-biphenyl]-4-yl)propanoic acid or substituted (S)-2-amin o-3-(4'-methoxy-2'-methyl-[1,1'-biphenyl]-4-yl)propanamide
XXGTXTSDX

SEQ ID NO: 205 - protein, 9 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-methoxy-2'-methyl-[1, 1'-biphenyl]-4-yl)propanoic acid or substituted (S)-2-amin o-3-(4'-methoxy-2'-methyl-[1,1'-biphenyl]-4-yl)propanamide
XXGTXTSDX

SEQ ID NO: 206 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-15-oxo-3,6, 9,12-tetraoxa-16-azaicosan-20-yl)oxy)-2'-ethyl-[1,1'-biphe nyl]-4-yl)propanoic acid
XXGTXTSDX

SEQ ID NO: 207 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-27-oxo-3,6, 9,12,15,18,21,24-octaoxa-28-azadotriacontan-32-yl)oxy)-2'- ethyl-[1,1'-biphenyl]-4-yl)propanoic acid
XXGTXTSDX

SEQ ID NO: 208 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-39-oxo-3,6, 9,12,15,18,21,24,27,30,33,36-dodecaoxa-40-azatetratetracon tan-44-yl)oxy)-2'-ethyl-[1,1'-biphenyl]-4-yl)propanoic acid
XXGTXTSDX

SEQ ID NO: 209 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
XXGTXTSDX

SEQ ID NO: 210 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-((1-((1R,8S,9s)-bicyclo[6.1.0]non -4-yn-9-yl)-3,17-dioxo-2,7,10,13,16-pentaoxa-4,18-diazadoc osan-22-yl)oxy)-2'-ethyl-[1,1'-biphenyl]-4-yl)propanoic acid
XXGTXTSDX

SEQ ID NO: 211 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 212 - protein, 5 aa, synthetic construct
  SITE 5: note = Residue attached to another sequence via an amide bond
GGGGS

SEQ ID NO: 213 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 214 - protein, 5 aa, synthetic construct
  SITE 5: note = Residue attached to another sequence via an amide bond
SGGGG

SEQ ID NO: 215 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-(4-(4-(13-amino-2,5,8 ,11-tetraoxatridecyl)-1H-1,2,3-triazol-1-yl)butoxy)-2'-eth yl-[1,1'-biphenyl]-4-yl)propanoic acid
XXGTXTSDX

SEQ ID NO: 216 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(1-((1R,8S,9s)-bicyclo[6.1. 0]non-4-yn-9-yl)-3-oxo-2,7,10,13,16-pentaoxa-4-azaheptadec an-17-yl)-1H-1,2,3-triazol-1-yl)butoxy)-2'-ethyl-[1,1'-bip henyl]-4-yl)propanoic acid
XXGTXTSDX

SEQ ID NO: 217 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (2S)-2-amino-3-(4'-(4-(4-(16-(cyclooct-2-yn-1-yloxy )-15-oxo-2,5,8,11-tetraoxa-14-azahexadecyl)-1H-1,2,3-triaz ol-1-yl)butoxy)-2'-ethyl-[1,1'-biphenyl]-4-yl)propanoic acid
XXGTXTSDX

SEQ ID NO: 218 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
XXGTXTSDX

SEQ ID NO: 219 - protein, 8 aa, synthetic construct
  SITE 1: note = (S)-2-(6-(1H-imidazol-5-yl)hexanamido)-3-(2H-tetraz ol-5-yl)propanoic acid
  SITE 4: note = (S)-2-amino-3-(2-fluorophenyl)-2-methylpropanoic acid
  VARIANT 8: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
XGTXTSDX

SEQ ID NO: 220 - protein, 8 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  VARIANT 8: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
XGTXTSDX

SEQ ID NO: 221 - protein, 8 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanoic acid side chain attached to another sequence via an amide bond
XGTXTSDX

SEQ ID NO: 222 - protein, 5 aa, synthetic construct
  SITE 5: note = Residue attached to another sequence via an amide bond
GGGGS

SEQ ID NO: 223 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 224 - protein, 10 aa, synthetic construct
  SITE 10: note = Residue attached to another sequence via an amide bond
GGGGSGGGGS

SEQ ID NO: 225 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 226 - protein, 6 aa, synthetic construct
  SITE 1: note = 3-(((((1R,8S,9s)-bicyclo[6.1.0]non-4-yn-9-yl)methox y)carbonyl)amino)propanoic acid
  SITE 2: note = O-((2R,3R,4S,5S,6R)-3,4,5-trihydroxy-6-(hydroxymeth yl)tetrahydro-2H-pyran-2-yl)-L-serine
  SITE 6: note = Residue attached to another sequence via an amide bond
XXGGGG

SEQ ID NO: 227 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 228 - protein, 5 aa, synthetic construct
  SITE 1: note = (2-(cyclooct-2-yn-1-yloxy)acetyl)glycine
  SITE 5: note = Residue attached to another sequence via an amide bond
XGGGS

SEQ ID NO: 229 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 230 - protein, 5 aa, synthetic construct
  SITE 1: note = O-((2R,3R,4S,5S,6R)-3,4,5-trihydroxy-6-(hydroxymeth yl)tetrahydro-2H-pyran-2-yl)-L-serine
  SITE 5: note = Residue attached to another sequence via an amide bond
XGGGG

SEQ ID NO: 231 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanoic acid
XXGTXTSDX

SEQ ID NO: 232 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-(4-(4-(1-((5aS,6R,6aR)-1 ,4,5,5a,6,6a,7,8-octahydrocyclopropa[5,6]cycloocta[1,2-d][ 1,2,3]triazol-6-yl)-3-oxo-2,7,10,13,16-pentaoxa-4-azahepta decan-17-yl)-1H-1,2,3-triazol-1-yl)butoxy)-[1,1'-biphenyl] -4-yl)propanoic acid
XXGTXTSDX

SEQ ID NO: 233 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-((1-((5aS,6R,6aR)-1,4,5, 5a,6,6a,7,8-octahydrocyclopropa[5,6]cycloocta[1,2-d][1,2,3 ]triazol-6-yl)-3,17-dioxo-2,7,10,13,16-pentaoxa-4,18-diaza docosan-22-yl)oxy)-[1,1'-biphenyl]-4-yl)propanoic acid
XXGTXTSDX

SEQ ID NO: 234 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanoic acid side chain attached to another sequence via an amide bond
XXGTXTSDX

SEQ ID NO: 235 - protein, 5 aa, synthetic construct
  SITE 1: note = (7-(3,9-dihydro-8H-dibenzo[b,f][1,2,3]triazolo[4,5- d]azocin-8-yl)-4,7-dioxoheptanoyl)glycine
  SITE 5: note = Residue attached to another sequence via an amide bond
XGGGS

SEQ ID NO: 236 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-(4-(4-(25-amino-2,5,8 ,11,14,17,20,23-octaoxapentacosyl)-1H-1,2,3-triazol-1-yl)b utoxy)-2'-ethyl-[1,1'-biphenyl]-4-yl)propanoic acid
XXGTXTSDX

SEQ ID NO: 237 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(30-(3,9-dihydro-8H-dibenzo [b,f][1,2,3]triazolo[4,5-d]azocin-8-yl)-27,30-dioxo-2,5,8, 11,14,17,20,23-octaoxa-26-azatriacontyl)-1H-1,2,3-triazol- 1-yl)butoxy)-2'-ethyl-[1,1'-biphenyl]-4-yl)propanoic acid
XXGTXTSDX

SEQ ID NO: 238 - protein, 10 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
YXXGTXTSDX

SEQ ID NO: 239 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
XXGTXTSDX

SEQ ID NO: 240 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(37-amino-2,5,8,11,14,17,20 ,23,26,29,32,35-dodecaoxaheptatriacontyl)-1H-1,2,3-triazol -1-yl)butoxy)-2'-ethyl-[1,1'-biphenyl]-4-yl)propanoic acid
XXGTXTSDX

SEQ ID NO: 241 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-30-amino-1-(1-(4-((4'-((S)-2-amino-2-carboxyeth yl)-2-ethyl-[1,1'-biphenyl]-4-yl)oxy)butyl)-1H-1,2,3-triaz ol-4-yl)-27-oxo-2,5,8,11,14,17,20,23-octaoxa-26-azahentria contan-31-oic acid
XXGTXTSDX

SEQ ID NO: 242 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-1-(1-(4-((4'-(2-amino-2-carboxyethyl)-2-ethyl-[ 1,1'-biphenyl]-4-yl)oxy)butyl)-1H-1,2,3-triazol-4-yl)-2,5, 8,11,14,17,20,23-octaoxahexacosan-26-oic acid side chain attached to another sequence via an amide bond
XXGTXTSDX

SEQ ID NO: 243 - protein, 6 aa, synthetic construct
  SITE 1: note = Residue attached to another sequence via an amide bond
GGTEPL

SEQ ID NO: 244 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = ((S)-2-amino-3-(4'-(4-(4-(25-amino-2,5,8,11,14,17,2 0,23-octaoxapentacosyl)-1H-1,2,3-triazol-1-yl)butoxy)-2'-e thyl-[1,1'-biphenyl]-4-yl)propanoic acid side chain attached to another sequence via an amide bond
XXGTXTSDX

SEQ ID NO: 245 - protein, 6 aa, synthetic construct
  SITE 1: note = ((benzyloxy)carbonyl)-L-leucine
  SITE 3: note = Residue side chain attached to another sequence via an amide bond
XLQGSG

SEQ ID NO: 246 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 247 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 248 - protein, 5 aa, synthetic construct
  SITE 5: note = Residue attached to another sequence via an amide bond
SGGGG

SEQ ID NO: 249 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 250 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 251 - protein, 9 aa, synthetic construct
  SITE 9: note = Residue attached to another sequence via an amide bond
GGGGSGGGG

SEQ ID NO: 252 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 253 - protein, 14 aa, synthetic construct
  SITE 14: note = Residue attached to another sequence via an amide bond
GGGGSGGGGSGGGG

SEQ ID NO: 254 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 255 - protein, 11 aa, synthetic construct
  SITE 11: note = Residue attached to another sequence via an amide bond
GGSGGSGGSGG

SEQ ID NO: 256 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 257 - protein, 5 aa, synthetic construct
  SITE 5: note = Residue attached to another sequence via an amide bond
GGGGS

SEQ ID NO: 258 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanoic acid side chain attached to another sequence via an amide bond
XXGTXTSDX

SEQ ID NO: 259 - protein, 9 aa, synthetic construct
  SITE 9: note = Residue attached to another sequence via an amide bond
GGGGSGGGG

SEQ ID NO: 260 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(55-amino-39,48-dioxo-2,5,8 ,11,14,17,20,23,26,29,32,35,41,44,50,53-hexadecaoxa-38,47- diazapentapentacontyl)-1H-1,2,3-triazol-1-yl)butoxy)-2'-et hyl-[1,1'-biphenyl]-4-yl)propanoic acid
XXGTXTSDX

SEQ ID NO: 261 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(43-amino-27,36-dioxo-2,5,8 ,11,14,17,20,23,29,32,38,41-dodecaoxa-26,35-diazatritetrac ontyl)-1H-1,2,3-triazol-1-yl)butoxy)-2'-ethyl-[1,1'-biphen yl]-4-yl)propanoic acid
XXGTXTSDX

SEQ ID NO: 262 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
XXGTXTSDX

SEQ ID NO: 263 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-15-oxo-3,6, 9,12-tetraoxa-16-azaicosan-20-yl)oxy)-2'-ethyl-[1,1'-biphe nyl]-4-yl)propanoic acid
XXGTXTSDX

SEQ ID NO: 264 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-27-oxo-3,6, 9,12,15,18,21,24-octaoxa-28-azadotriacontan-32-yl)oxy)-2'- ethyl-[1,1'-biphenyl]-4-yl)propanamide
XXGTXTSDX

SEQ ID NO: 265 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-39-oxo-3,6, 9,12,15,18,21,24,27,30,33,36-dodecaoxa-40-azatetratetracon tan-44-yl)oxy)-2'-ethyl-[1,1'-biphenyl]-4-yl)propanamide
XXGTXTSDX

SEQ ID NO: 266 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanamide
XXGTXTSDX

SEQ ID NO: 267 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-((1-((1R,8S,9s)-bicyclo[6.1.0]non -4-yn-9-yl)-3,17-dioxo-2,7,10,13,16-pentaoxa-4,18-diazadoc osan-22-yl)oxy)-2'-ethyl-[1,1'-biphenyl]-4-yl)propanamide
XXGTXTSDX

SEQ ID NO: 268 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 269 - protein, 5 aa, synthetic construct
  SITE 5: note = Residue attached to another sequence via an amide bond
GGGGS

SEQ ID NO: 270 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 271 - protein, 5 aa, synthetic construct
  SITE 5: note = Residue attached to another sequence via an amide bond
SGGGG

SEQ ID NO: 272 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-(4-(4-(13-amino-2,5,8 ,11-tetraoxatridecyl)-1H-1,2,3-triazol-1-yl)butoxy)-2'-eth yl-[1,1'-biphenyl]-4-yl)propanamide
XXGTXTSDX

SEQ ID NO: 273 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(1-((1R,8S,9s)-bicyclo[6.1. 0]non-4-yn-9-yl)-3-oxo-2,7,10,13,16-pentaoxa-4-azaheptadec an-17-yl)-1H-1,2,3-triazol-1-yl)butoxy)-2'-ethyl-[1,1'-bip henyl]-4-yl)propanamide
XXGTXTSDX

SEQ ID NO: 274 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (2S)-2-amino-3-(4'-(4-(4-(16-(cyclooct-2-yn-1-yloxy )-15-oxo-2,5,8,11-tetraoxa-14-azahexadecyl)-1H-1,2,3-triaz ol-1-yl)butoxy)-2'-ethyl-[1,1'-biphenyl]-4-yl)propanamide
XXGTXTSDX

SEQ ID NO: 275 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanamide
XXGTXTSDX

SEQ ID NO: 276 - protein, 8 aa, synthetic construct
  SITE 1: note = (S)-2-(6-(1H-imidazol-5-yl)hexanamido)-3-(2H-tetraz ol-5-yl)propanoic acid
  SITE 4: note = (S)-2-amino-3-(2-fluorophenyl)-2-methylpropanoic acid
  VARIANT 8: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanamide
XGTXTSDX

SEQ ID NO: 277 - protein, 8 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  VARIANT 8: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanamide
XGTXTSDX

SEQ ID NO: 278 - protein, 8 aa, synthetic construct
  SITE 1: note = (S)-2-(3-amino-2,2-dimethyl-3-oxopropanamido)-3-(2H -tetrazol-5-yl)propanoic acid
  SITE 4: note = (S)-2-amino-3-(2-fluorophenyl)-2-methylpropanoic acid
  SITE 8: note = (S)-2-amino-3-(4'-(4-aminobutoxy)-2'-ethyl-[1,1'-bi phenyl]-4-yl)propanamide side chain attached to another sequence via an amide bond
XGTXTSDX

SEQ ID NO: 279 - protein, 5 aa, synthetic construct
  SITE 5: note = Residue attached to another sequence via an amide bond
GGGGS

SEQ ID NO: 280 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 281 - protein, 10 aa, synthetic construct
  SITE 10: note = Residue attached to another sequence via an amide bond
GGGGSGGGGS

SEQ ID NO: 282 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 283 - protein, 6 aa, synthetic construct
  SITE 1: note = 3-(((((1R,8S,9s)-bicyclo[6.1.0]non-4-yn-9-yl)methox y)carbonyl)amino)propanoic acid
  SITE 2: note = O-((2R,3R,4S,5S,6R)-3,4,5-trihydroxy-6-(hydroxymeth yl)tetrahydro-2H-pyran-2-yl)-L-serine
  SITE 6: note = Residue attached to another sequence via an amide bond
XXGGGG

SEQ ID NO: 284 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 285 - protein, 5 aa, synthetic construct
  SITE 1: note = (2-(cyclooct-2-yn-1-yloxy)acetyl)glycine
  SITE 5: note = Residue attached to another sequence via an amide bond
XGGGS

SEQ ID NO: 286 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 287 - protein, 5 aa, synthetic construct
  SITE 1: note = O-((2R,3R,4S,5S,6R)-3,4,5-trihydroxy-6-(hydroxymeth yl)tetrahydro-2H-pyran-2-yl)-L-serine
  SITE 5: note = Residue attached to another sequence via an amide bond
XGGGG

SEQ ID NO: 288 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-((1-amino-75-oxo-3,6, 9,12,15,18,21,24,27,30,33,36,39,42,45,48,51,54,57,60,63,66 ,69,72-tetracosaoxa-76-azaoctacontan-80-yl)oxy)-2'-ethyl-[ 1,1'-biphenyl]-4-yl)propanamide
XXGTXTSDX

SEQ ID NO: 289 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-(4-(4-(1-((5aS,6R,6aR)-1 ,4,5,5a,6,6a,7,8-octahydrocyclopropa[5,6]cycloocta[1,2-d][ 1,2,3]triazol-6-yl)-3-oxo-2,7,10,13,16-pentaoxa-4-azahepta decan-17-yl)-1H-1,2,3-triazol-1-yl)butoxy)-[1,1'-biphenyl] -4-yl)propanamide
XXGTXTSDX

SEQ ID NO: 290 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(2'-ethyl-4'-((1-((5aS,6R,6aR)-1,4,5, 5a,6,6a,7,8-octahydrocyclopropa[5,6]cycloocta[1,2-d][1,2,3 ]triazol-6-yl)-3,17-dioxo-2,7,10,13,16-pentaoxa-4,18-diaza docosan-22-yl)oxy)-[1,1'-biphenyl]-4-yl)propanamide
XXGTXTSDX

SEQ ID NO: 291 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-aminobutoxy)-2'-ethyl-[1,1'-bi phenyl]-4-yl)propanamide side chain attached to another sequence via an amide bond
XXGTXTSDX

SEQ ID NO: 292 - protein, 5 aa, synthetic construct
  SITE 1: note = (7-(3,9-dihydro-8H-dibenzo[b,f][1,2,3]triazolo[4,5- d]azocin-8-yl)-4,7-dioxoheptanoyl)glycine
  SITE 5: note = Residue attached to another sequence via an amide bond
XGGGS

SEQ ID NO: 293 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-(4-(4-(25-amino-2,5,8 ,11,14,17,20,23-octaoxapentacosyl)-1H-1,2,3-triazol-1-yl)b utoxy)-2'-ethyl-[1,1'-biphenyl]-4-yl)propanamide
XXGTXTSDX

SEQ ID NO: 294 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(30-(3,9-dihydro-8H-dibenzo [b,f][1,2,3]triazolo[4,5-d]azocin-8-yl)-27,30-dioxo-2,5,8, 11,14,17,20,23-octaoxa-26-azatriacontyl)-1H-1,2,3-triazol- 1-yl)butoxy)-2'-ethyl-[1,1'-biphenyl]-4-yl)propanamide
XXGTXTSDX

SEQ ID NO: 295 - protein, 10 aa, synthetic construct
  SITE 2: note = 2-amino-2-methylpropanoic acid
  SITE 3: note = (S)-2-amino-3-(2H-tetrazol-5-yl)propanoic acid
  SITE 6: note = (S)-2-amino-3-(2-fluorophenyl)-2-methylpropanoic acid
  SITE 10: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanamide
YXXGTXTSDX

SEQ ID NO: 296 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanamide
XXGTXTSDX

SEQ ID NO: 297 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(37-amino-2,5,8,11,14,17,20 ,23,26,29,32,35-dodecaoxaheptatriacontyl)-1H-1,2,3-triazol -1-yl)butoxy)-2'-ethyl-[1,1'-biphenyl]-4-yl)propanamide
XXGTXTSDX

SEQ ID NO: 298 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-30-amino-1-(1-(4-((4'-((S)-2-amino-2-carboxyeth yl)-2-ethyl-[1,1'-biphenyl]-4-yl)oxy)butyl)-1H-1,2,3-triaz ol-4-yl)-27-oxo-2,5,8,11,14,17,20,23-octaoxa-26-azahentria contan-31-oic acid
XXGTXTSDX

SEQ ID NO: 299 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-1-(1-(4-((4'-(2,3-diamino-3-oxopropyl)-2-ethyl- [1,1'-biphenyl]-4-yl)oxy)butyl)-1H-1,2,3-triazol-4-yl)-2,5 ,8,11,14,17,20,23-octaoxahexacosan-26-oic acid side chain attached to another sequence via an amide bond
XXGTXTSDX

SEQ ID NO: 300 - protein, 6 aa, synthetic construct
  SITE 1: note = Residue attached to another sequence via an amide bond
GGTEPL

SEQ ID NO: 301 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = ((S)-2-amino-3-(4'-(4-(4-(25-amino-2,5,8,11,14,17,2 0,23-octaoxapentacosyl)-1H-1,2,3-triazol-1-yl)butoxy)-2'-e thyl-[1,1'-biphenyl]-4-yl)propanamide side chain attached to another sequence via an amide bond
XXGTXTSDX

SEQ ID NO: 302 - protein, 6 aa, synthetic construct
  SITE 1: note = ((benzyloxy)carbonyl)-L-leucine
  SITE 3: note = Residue side chain attached to another sequence via an amide bond
XLQGSG

SEQ ID NO: 303 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 304 - protein, 5 aa, synthetic construct
  SITE 5: note = Residue attached to another sequence via an amide bond
GGGGS

SEQ ID NO: 305 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 306 - protein, 5 aa, synthetic construct
  SITE 5: note = Residue attached to another sequence via an amide bond
SGGGG

SEQ ID NO: 307 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 308 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 309 - protein, 9 aa, synthetic construct
  SITE 9: note = Residue attached to another sequence via an amide bond
GGGGSGGGG

SEQ ID NO: 310 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 311 - protein, 14 aa, synthetic construct
  SITE 14: note = Residue attached to another sequence via an amide bond
GGGGSGGGGSGGGG

SEQ ID NO: 312 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 313 - protein, 11 aa, synthetic construct
  SITE 11: note = Residue attached to another sequence via an amide bond
GGSGGSGGSGG

SEQ ID NO: 314 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 315 - protein, 5 aa, synthetic construct
  SITE 5: note = Residue attached to another sequence via an amide bond
GGGGS

SEQ ID NO: 316 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(aminomethyl)-1H-1,2,3-tria zol-1-yl)butoxy)-2'-ethyl-[1,1'-biphenyl]-4-yl)propanamide side chain attached to another sequence via an amide bond
XXGTXTSDX

SEQ ID NO: 317 - protein, 9 aa, synthetic construct
  SITE 9: note = Residue attached to another sequence via an amide bond
GGGGSGGGG

SEQ ID NO: 318 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(55-amino-39,48-dioxo-2,5,8 ,11,14,17,20,23,26,29,32,35,41,44,50,53-hexadecaoxa-38,47- diazapentapentacontyl)-1H-1,2,3-triazol-1-yl)butoxy)-2'-et hyl-[1,1'-biphenyl]-4-yl)propanamide
XXGTXTSDX

SEQ ID NO: 319 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(43-amino-27,36-dioxo-2,5,8 ,11,14,17,20,23,29,32,38,41-dodecaoxa-26,35-diazatritetrac ontyl)-1H-1,2,3-triazol-1-yl)butoxy)-2'-ethyl-[1,1'-biphen yl]-4-yl)propanamide
XXGTXTSDX

SEQ ID NO: 320 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanamide
XXGTXTSDX

SEQ ID NO: 321 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-methyl-4'-methoxy-[1, 1'-biphenyl]-4-yl)propanoic acid linker or Substituted (S)-2-amino-3-(2'-methyl-4'-methoxy-[1,1'-biphenyl]-4-yl)p ropanamide linker
XGTXTSDX

SEQ ID NO: 322 - protein, 9 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid linker
  VARIANT 9: note = (S)-2-amino-5-(3,5-dimethylphenyl)pentanoic acid, or substituted (S)-2-amino-5-phenylpentanoic acid or (S)-2-amino-5-(3,5-dimethylphenyl)pentanamide, or substituted (S)-2-amino-5-phenylpentanamide
XGTXTSDXX

SEQ ID NO: 323 - protein, 9 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid
  VARIANT 9: note = substituted (S)-2-amino-5-phenylpentanoic acid linker or substituted (S)-2-amino-5-phenylpentanamide linker
XGTXTSDXX

SEQ ID NO: 324 - protein, 24 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  SITE 7: note = (S)-2-amino-3-(1H-tetrazol-5-yl)propanoic acid
  SITE 11: note = 2-amino-2-methylpropanoic acid
  SITE 13: note = (S)-2-amino-3-(1H-tetrazol-5-yl)propanoic acid
  VARIANT 18: note = Optionally substituted Ala linker
  SITE 20: note = (S)-2-amino-3-(2-fluorophenyl)-2-methylpropanoic acid
XGTXTSXYSIXLXKIAQXAXVQGG

SEQ ID NO: 325 - protein, 8 aa, synthetic construct
  VARIANT 1: note = Substituted (S)-2-amino-3-(2H-tetrazol-5-yl)propanoic acid or Gln
  SITE 4: note = (S)-2-amino-3-(2-fluorophenyl)-2-methylpropanoic acid
  VARIANT 8: note = Substituted (S)-2-amino-3-(2'-ethyl-4'-methoxy-[1,1 '-biphenyl]-4-yl)propanoic acid linker or Substituted (S)-2-amino-3-(2'-ethyl-4'-methoxy-[1,1'-biphenyl]-4-yl)pr opanamide linker
XGTXTSDX

SEQ ID NO: 326 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(2'-ethyl-4'-(4-(4-(metho xymethyl)-1H-1,2,3-triazol-1-yl)butoxy)-[1,1'-biphenyl]-4- yl)propanoic acid
  VARIANT 10: note = (S)-2-amino-5-(3,5-dimethylphenyl)pentanoic acid or (S)-2-amino-5-(3,5-dimethylphenyl)pentanamide
XXGTXTSDXX

SEQ ID NO: 327 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(2'-ethyl-4'-(4-(4-(metho xymethyl)-1H-1,2,3-triazol-1-yl)butoxy)-[1,1'-biphenyl]-4- yl)propanoic acid or substituted (S)-2-amino-3-(2'-ethyl-4 '-(4-(4-(methoxymethyl)-1H-1,2,3-triazol-1-yl)butoxy)-[1,1 '-biphenyl]-4-yl)propanamide
XXGTXTSDX

SEQ ID NO: 328 - protein, 9 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-methoxy-2'-methyl-[1, 1'-biphenyl]-4-yl)propanoic acid or substituted (S)-2-amin o-3-(4'-methoxy-2'-methyl-[1,1'-biphenyl]-4-yl)propanamide
XXGTXTSDX

SEQ ID NO: 329 - protein, 9 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(2'-ethyl-4'-methoxy-[1,1 '-biphenyl]-4-yl)propanoic acid or substituted (S)-2-amino -3-(2'-ethyl-4'-methoxy-[1,1'-biphenyl]-4-yl)propanamide
XXGTXTSDX

SEQ ID NO: 330 - protein, 6 aa, synthetic construct
  VARIANT 1: note = Substituted succinic acid
XGGGGS

SEQ ID NO: 331 - protein, 6 aa, synthetic construct
  VARIANT 1: note = Substituted succinic acid
XSGGGG

SEQ ID NO: 332 - protein, 11 aa, synthetic construct
  VARIANT 1: note = Substituted succinic acid
XGGGGSGGGGS

SEQ ID NO: 333 - protein, 7 aa, synthetic construct
  SITE 1: note = ((5aR,6S,6aS)-1,4,5,5a,6,6a,7,8-octahydrocyclopropa [5,6]cycloocta[1,2-d][1,2,3]triazol-6-yl)methyl carbamate
  SITE 2: note = 3-aminopropanoic acid
  SITE 3: note = O-((2R,3R,4S,5S,6R)-3,4,5-trihydroxy-6-(hydroxymeth yl)tetrahydro-2H-pyran-2-yl)-L-serine
XXXGGGG

SEQ ID NO: 334 - protein, 6 aa, synthetic construct
  SITE 1: note = 2-((4,5,6,7,8,9-hexahydro-1H-cycloocta[d][1,2,3]tri azol-9-yl)oxy)acetic acid
XGGGGS

SEQ ID NO: 335 - protein, 6 aa, synthetic construct
  VARIANT 1: note = Substituted succinic acid
  SITE 2: note = O-((2R,3R,4S,5S,6R)-3,4,5-trihydroxy-6-(hydroxymeth yl)tetrahydro-2H-pyran-2-yl)-L-serine
XXGGGG

SEQ ID NO: 336 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-((23-amino-3,6,9,12,15,18,21-hept aoxatricosyl)oxy)-2'-methyl-[1,1'-biphenyl]-4-yl)propanoic acid
XXGTXTSDX

SEQ ID NO: 337 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-((S)-5-(tert-butoxycarbonyl )-1-(9H-fluoren-9-yl)-3,8-dioxo-2,12,15,18,21,24,27,30,33- nonaoxa-4,9-diazatetratriacontan-34-yl)-1H-1,2,3-triazol-1 -yl)butoxy)-2'-ethyl-[1,1'-biphenyl]-4-yl)propanoic acid
XXGTXTSDX

SEQ ID NO: 338 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-30-((((9H-fluoren-9-yl)methoxy)carbonyl)amino)- 1-(1-(4-((4'-((S)-2-amino-2-carboxyethyl)-2-ethyl-[1,1'-bi phenyl]-4-yl)oxy)butyl)-1H-1,2,3-triazol-4-yl)-27-oxo-2,5, 8,11,14,17,20,23-octaoxa-26-azahentriacontan-31-oic acid
XXGTXTSDX

SEQ ID NO: 339 - protein, 9 aa, synthetic construct
  SITE 1: note = 2,2-dimethyl-3-oxo-3-((2-(1-trityl-1H-imidazol-5-yl )ethyl)amino)propanoic acid
  SITE 2: note = (S)-2-amino-3-(1-(2,4-dimethoxybenzyl)-1H-tetrazol- 5-yl)propanoic acid
  SITE 5: note = (S)-2-amino-3-(2-fluorophenyl)-2-methylpropanoic acid
  SITE 8: note = (S)-2-amino-4-(tert-butoxy)-4-oxobutanoic acid
  SITE 9: note = (S)-2-amino-3-(4'-(4-(4-(29,29-dimethyl-27-oxo-2,5, 8,11,14,17,20,23,28-nonaoxa-26-azatriacontyl)-1H-1,2,3-tri azol-1-yl)butoxy)-2'-ethyl-[1,1'-biphenyl]-4-yl)propanoic acid attached to a resin
XXGTXTSXX

SEQ ID NO: 340 - protein, 9 aa, synthetic construct
  SITE 1: note = 2,2-dimethyl-3-oxo-3-((2-(1-trityl-1H-imidazol-5-yl )ethyl)amino)propanoic acid
  SITE 2: note = (S)-2-amino-3-(1-(2,4-dimethoxybenzyl)-1H-tetrazol- 5-yl)propanoic acid
  SITE 5: note = (S)-2-amino-3-(2-fluorophenyl)-2-methylpropanoic acid
  SITE 8: note = (S)-2-amino-4-(tert-butoxy)-4-oxobutanoic acid
  SITE 9: note = (S)-2-amino-3-(4'-(4-(4-(29,29-dimethyl-27-oxo-2,5, 8,11,14,17,20,23,28-nonaoxa-26-azatriacontyl)-1H-1,2,3-tri azol-1-yl)butoxy)-2'-ethyl-[1,1'-biphenyl]-4-yl)propanoic acid
XXGTXTSXX

SEQ ID NO: 341 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-(25-amino-2,5,8,11,14,17,20 ,23-octaoxapentacosyl)-1H-1,2,3-triazol-1-yl)butoxy)-2'-et hyl-[1,1'-biphenyl]-4-yl)propanoic acid
XXGTXTSDX

SEQ ID NO: 342 - protein, 9 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-((30S,33S,36S)-30-(tert-but oxycarbonyl)-33,36-diisobutyl-40,40-dimethyl-27,32,35,38-t etraoxo-2,5,8,11,14,17,20,23,39-nonaoxa-26,31,34,37-tetraa zahentetracontyl)-1H-1,2,3-triazol-1-yl)butoxy)-2'-ethyl-[ 1,1'-biphenyl]-4-yl)propanoic acid
XXGTXTSDX

SEQ ID NO: 343 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 7: note = O-(tert-butyl)-L-serine
  SITE 9: note = (S)-2-amino-3-(4'-(4-azidobutoxy)-2'-ethyl-[1,1'-bi phenyl]-4-yl)propanoic acid
  SITE 10: note = (S)-2-amino-5-(3,5-dimethylphenyl)pentanoic acid
XXGTXTXDXX

SEQ ID NO: 344 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-((S)-5-(tert-butoxycarbonyl )-1-(9H-fluoren-9-yl)-3,8-dioxo-2,12,15,18,21,24,27,30,33- nonaoxa-4,9-diazatetratriacontan-34-yl)-1H-1,2,3-triazol-1 -yl)butoxy)-2'-ethyl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 345 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = ((S)-2-amino-3-(4'-(4-(4-((S)-30-amino-33,33-dimeth yl-27,31-dioxo-2,5,8,11,14,17,20,23,32-nonaoxa-26-azatetra triacontyl)-1H-1,2,3-triazol-1-yl)butoxy)-2'-ethyl-[1,1'-b iphenyl]-4-yl)propanoic acid
  SITE 10: note = (S)-2-amino-5-(3,5-dimethylphenyl)pentanoic acid
XXGTXTSDXX

SEQ ID NO: 346 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-(4-(4-((30S,33S,36S)-30-(tert-but oxycarbonyl)-33,36-diisobutyl-40,40-dimethyl-27,32,35,38-t etraoxo-2,5,8,11,14,17,20,23,39-nonaoxa-26,31,34,37-tetraa zahentetracontyl)-1H-1,2,3-triazol-1-yl)butoxy)-2'-ethyl-[ 1,1'-biphenyl]-4-yl)propanoic acid
  SITE 10: note = (S)-2-amino-5-(3,5-dimethylphenyl)pentanoic acid
XXGTXTSDXX

SEQ ID NO: 347 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-30-amino-1-(1-(4-((4'-((S)-2-amino-2-carboxyeth yl)-2-ethyl-[1,1'-biphenyl]-4-yl)oxy)butyl)-1H-1,2,3-triaz ol-4-yl)-27-oxo-2,5,8,11,14,17,20,23-octaoxa-26-azahentria contan-31-oic acid
  SITE 10: note = (S)-2-amino-5-(3,5-dimethylphenyl)pentanamide
XXGTXTSDXX

SEQ ID NO: 348 - protein, 9 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = (S)-2-amino-3-(4'-((23-amino-3,6,9,12,15,18,21-hept aoxatricosyl)oxy)-2'-ethyl-[1,1'-biphenyl]-4-yl)propanoic acid or (S)-2-amino-3-(4'-((23-amino-3,6,9,12,15,18,21-hep taoxatricosyl)oxy)-2'-ethyl-[1,1'-biphenyl]-4-yl)propanami de
XXGTXTSDX

SEQ ID NO: 349 - protein, 9 aa, synthetic construct
  SITE 1: note = 2-(3-(1H-imidazol-5-yl)propanamido)-2-methylpropano ic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = (S)-2-amino-3-(4'-((23-amino-3,6,9,12,15,18,21-hept aoxatricosyl)oxy)-2'-methyl-[1,1'-biphenyl]-4-yl)propanoic acid or (S)-2-amino-3-(4'-((23-amino-3,6,9,12,15,18,21-hep taoxatricosyl)oxy)-2'-methyl-[1,1'-biphenyl]-4-yl)propanam ide
XXGTXTSDX

SEQ ID NO: 350 - protein, 6 aa, synthetic construct
  SITE 3: note = Residue side chain attached to another sequence
LLQGSG

SEQ ID NO: 351 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  VARIANT 9: note = Substituted (S)-2-amino-3-(4'-(4-(4-(13-amino-2,5,8 ,11-tetraoxatridecyl)-1H-1,2,3-triazol-1-yl)butoxy)-2'-eth yl-[1,1'-biphenyl]-4-yl)propanoic acid
  SITE 10: note = (S)-2-amino-5-(3,5-dimethylphenyl)pentanoic acid
XXGTXTSDXX

SEQ ID NO: 352 - protein, 5 aa, synthetic construct
  SITE 5: note = Residue attached to another sequence via an amide bond
GGGGS

SEQ ID NO: 353 - protein, 8 aa, synthetic construct
  SITE 8: note = Residue attached to another sequence via an amide bond
GGSGGSGG

SEQ ID NO: 354 - protein, 10 aa, synthetic construct
  SITE 1: note = 3-((2-(1H-imidazol-5-yl)ethyl)amino)-2,2-dimethyl-3 -oxopropanoic acid
  SITE 2: note = (S)-2-amino-3-(2H-tetrazol-5-yl)propanoic acid
  SITE 5: note = (S)-2-amino-3-(2-fluorophenyl)-2-methylpropanoic acid
  SITE 9: note = 30-amino-1-(1-(4-((4'((S)-2-amino-2-carboxyethyl)-2 -ethyl-[1,1'-biphenyl]-4-yl)oxy)butyl)-1H-1,2,3-triazol-4- yl)-27-oxo-2,5,8,11,14,17,20,23-octaoxa-26-azahentriaconta n-31-oic acid
  SITE 10: note = (S)-2-amino-5-(3,5-dimethylphenyl)pentanoic acid
XXGTXTSDXX